(19) United States
(12) Patent Application Publication
(10) Pub. No.: US 2020/0385478 A1
(43) Pub. Date: Dec. 10, 2020

(54) COMPOSITIONS AND METHODS FOR CANCER IMMUNOTHERAPY

(71) Applicant: BIOGRAPH 55, INC., South San Francisco, CA (US)

(72) Inventor: Paul C. Tumeh, San Francisco, CA (US)

(21) Appl. No.: 16/959,619

(22) PCT Filed: Jan. 4, 2019
(86) PCT No.: PCT/US2019/012429

§ 371 Date: Jul. 1, 2020

Related U.S. Application Data
(60) Provisional application No. 62/614,294, filed on Jan. 5, 2018.

(51) Int. Cl. C07K 16/28; A61P 35/00; A61P 35/02

(52) U.S. Cl. CPC C07K 16/2875; C07K 16/2818; C07K 16/2827; A61P 35/00; A61K 2039/54; C07K 16/2803; C07K 2317/31; C07K 2317/24; C07K 2317/41; A61P 35/02

(57) ABSTRACT

Provided herein are analytical methods to be applied to samples, diagnostic methods, methods of determining a therapeutic regimens, and methods of treating cancer in a subject comprising compositions described herein.

### CROSS REFERENCE

This application claims priority to U.S. Application No. 62/614,294, filed Jan. 5, 2018, which is entirely incorporated herein by reference.

### BACKGROUND

Cancer is a leading cause of death throughout the world. One approach to cancer treatment is cancer immunotherapy. Cancer immunotherapy involves the use of compositions and methods to elicit or enhance an individual's immune system against cancerous cells.

### BRIEF SUMMARY

Disclosed herein are methods comprising administering to a subject, a therapeutically effective amount of a B-cell activating factor (BAFF) inhibitor or a salt thereof and a therapeutically effective amount of an immune checkpoint inhibitor or salt thereof. In some embodiments, the BAFF inhibitor or a salt thereof can comprise an antibody, single chain antibody molecule, or an active fragment thereof. In some embodiments, the antibody or an active fragment thereof can be a monoclonal antibody or an active fragment thereof or a polyclonal antibody or an active fragment thereof. In some embodiments, the antibody or an active fragment thereof can be a monoclonal antibody or an active fragment thereof. In some embodiments, the antibody or an active fragment thereof can be bispecific. In some embodiments, the antibody or an active fragment thereof can comprise a first domain that binds BAFF or B-cell activating factor receptor (BAFF-R). In some embodiments, the antibody or an active fragment thereof can comprise a second domain that binds PD-1 or PD-L1. In some embodiments, the antibody or an active fragment thereof can be humanized. In some embodiments, the antibody or an active fragment thereof can be an IgG, IgE, IgM, IgD, IgA or IgY. In some embodiments, the antibody or an active fragment thereof can be a recombinant protein. In some embodiments, the BAFF inhibitor or a salt thereof can comprise a heavy chain having at least 80% sequence identity with a sequence shown in SEQ ID NO: 150. In some embodiments, the BAFF inhibitor or a salt thereof can comprise a heavy chain having at least 90% sequence identity with a sequence shown in SEQ ID NO: 150. In some embodiments, the BAFF inhibitor or a salt thereof can comprise a light chain having at least 80% sequence identity with a sequence shown in SEQ ID NO: 151. In some embodiments, the BAFF inhibitor or a salt thereof can comprise a light chain having at least 90% sequence identity with a sequence shown in SEQ ID NO:151. In some embodiments, the BAFF inhibitor or a salt thereof can comprise a light chain variable region having at least 80% sequence identity with a sequence shown in SEQ ID NO: 153. In some embodiments, the BAFF inhibitor or a salt thereof can comprise a heavy chain variable region having at least 80% sequence identity with a sequence shown in SEQ ID NO: 152. In some embodiments, the BAFF inhibitor or a salt thereof can comprise a heavy chain having at least 80% sequence identity with a sequence shown in SEQ ID NO: 154. In some embodiments, the BAFF inhibitor or a salt thereof can comprise a heavy chain having at least 90% sequence identity with a sequence shown in SEQ ID NO: 154. In some embodiments, the BAFF inhibitor or a salt thereof can comprise a light chain having at least 80% sequence identity with a sequence shown in SEQ ID NO: 155. In some embodiments, the BAFF inhibitor or a salt thereof can comprise a light chain having at least 90% sequence identity with a sequence shown in SEQ ID NO: 155. In some embodiments, the BAFF inhibitor or a salt thereof can comprise a light chain variable region having at least 80% sequence identity with a sequence shown in SEQ ID NO: 157. In some embodiments, the BAFF inhibitor or a salt thereof can comprise a heavy chain variable having at least 90% sequence identity with a sequence shown in SEQ ID NO: 156. In some embodiments, the BAFF inhibitor or a salt thereof can comprise a sequence having at least 80% length homology to a sequence shown in SEQ ID NO:1-16 or SEQ ID NO:150-163. In some embodiments, the BAFF inhibitor or a salt thereof can be a biosimilar product to a reference product, wherein the reference product can comprise tabalumab, atacicept, RCT-18, BAFF/IL-17 bispecific antibody, ardenermin, briobacept, blisibimod, belimumab, MEDI-700, NOV-5, rGel/BlyS, a salt of any one thereof, and any combination thereof. In some embodiments, the BAFF inhibitor or a salt thereof can be an interchangeable product to a reference product, wherein the reference product can comprise tabalumab, atacicept, RCT-18, BAFF/IL-17 bispecific antibody, ardenermin, briobacept, blisibimod, belimumab, MEDI-700, NOV-5, rGel/BlyS, a salt of any one thereof, and any combination thereof. In some embodiments, the BAFF inhibitor or a salt thereof can comprise a posttranslational modification. In some embodiments, the posttranslational modification can comprise glycosylation, methylation, phosphorylation, lipidation, acetylation, nitrosylation, or any combination thereof. In some embodiments, the posttranslational modification can comprise the glycosylation. In some embodiments, the BAFF inhibitor or a salt thereof can bind a polypeptide having at least 80% sequence identity with a sequence shown in SEQ ID NOs: 31, 32 or 33. In some embodiments, the BAFF inhibitor or a salt thereof can bind a polypeptide having at least 80% sequence identity with a sequence shown in SEQ ID NOs: 149. In some embodiments, the BAFF or a BAFF-R inhibitor can comprise an aptamer comprising 100% homology, 95% homology, 90% homology, 85% homology, 80% homology, 75% homology, 70% homology, 65% homology, 60% homology, 55% homology, 50% homology, to an entire length or a fraction of an entire length of Aptamer 1 (SEQ ID NO: 175) 5′-GGG AGG ACG AUG CGG GAG GCU CAA CAA UGA UAG AGC CCG CAA UGU UGA UAG UUG UGC CCA GUC UGC AGA CGA CUC GCC CGA-3′; Aptamer 2 (SEQ ID NO: 176) 5′-GGG AGG ACG AUG CGG AUA ACU AUU GUG CUA GAG GGC UUA UUU AUG UGA GCC GGU UGA UAG UUG CGC AGA CGA CUC GCC CGA-3′; or Aptamer 3 (SEQ ID NO: 177) 5′-GGG AGG ACG AUG CGG AUC CUC CGA AGG UCG CGC CAA CGU CAC ACA UUA AGC UUU UGU UCG UCU GCA GAC GAC UCG CCC GA-3′. In some embodiments, the subject can have a cancer or is suspected of having the cancer. In some embodiments, the cancer can comprise leukemia, melanoma, prostate cancer, bladder cancer, osteosarcoma, cervical cancer, liver cancer, multiple myeloma, testicular, renal cancer or a combination thereof. In some embodiments, the cancer can be at least partially refractive to an immune checkpoint inhibitor or a salt thereof. In some embodiments, the cancer was or can be diagnosed by an assay. In some embodiments, the assay can be a companion diagnostic. In some embodiments, the immune checkpoint inhibitor can inhibit PD-1, PD-L1, PD-L2, CTLA-4, A2AR, B7-H3, B7-H4, BTLA, IDO, KIR, LAG3, TIM-3, VISTA, CD160, TIGIT or PSGL-1. In some embodiments, the immune checkpoint inhibitor or a salt thereof can comprise a PD-1 inhibitor or a salt thereof. In some embodiments, the PD-1 inhibitor or a salt thereof can comprise nivolumab, pembrolizumab, pidilizumab, BGB-A31, AMP-224, MEDI680 (AMP-514), PDR001, Cemiplimab or a salt of any one thereof. In some embodiments, the immune checkpoint inhibitor or a salt thereof can comprise a PD-L1 inhibitor or a salt thereof. In some embodiments, the PD-L1 inhibitor or a salt thereof can comprise nivolumab, docetaxel, pembrolizumab, pidilizumab, BGB-A31, MEDI0680 (AMP-514), or a salt of any one thereof. In some embodiments, the PD-1 or PD-L1 inhibitor may be any one of structure 1-7, or a analogue or a derivative thereof. In some embodiments, the PD-1 or PD-L1 inhibitor may have the sequence of SEQ ID NO: 17, SEQ ID NO: 18, SEQ ID NO: 19, SEQ ID NO: 20, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, a fragment of any of these, a sequence having at least about: 60%, 65%, 70%, 75%, 80%, 85%, 90%, 95%, 99% or more sequence homology to any of these, or any combination thereof. In some embodiments, the BAFF inhibitor or a salt thereof can be administered intra-arterially, intravenously, intramuscularly, orally, subcutaneously, via inhalation, or any combination thereof. In some embodiments, the BAFF inhibitor or a salt thereof can be administered intravenously. In some embodiments, the therapeutically effective amount of the BAFF inhibitor or a salt thereof can comprise a dose of about 0.04 mg/kg to about 20 mg/kg relative to a body weight of the subject. In some embodiments, the immune checkpoint inhibitor or said salt thereof can be administered intra-arterially, intravenously, intramuscularly, orally, subcutaneously, via inhalation, or any combination thereof. In some embodiments, the immune checkpoint inhibitor or a salt thereof can be administered intravenously. In some embodiments, the therapeutically effective amount of the immune checkpoint inhibitor or a salt thereof can comprise a dose of about 0.04 mg/kg to about 20 mg/kg relative to a body weight of the subject. In some embodiments, the BAFF inhibitor or a salt thereof and the immune checkpoint inhibitor or the salt thereof can be administered concurrently. In some embodiments, the BAFF inhibitor or a salt thereof and the immune checkpoint inhibitor or a salt thereof can be administered together in a single composition. In some embodiments, the BAFF inhibitor or salt thereof and the immune checkpoint inhibitor or the salt thereof can be administered sequentially. In some embodiments, the BAFF inhibitor or a salt thereof can be first administered and the immune checkpoint inhibitor or salt thereof can administered second. In some embodiments, the immune checkpoint inhibitor or a salt thereof can be first administered and the BAFF inhibitor or the salt thereof can be administered second. In some embodiments, administering can be performed at least once a month. In some embodiments, administering can be performed at least twice a month. In some embodiments, the subject can be a human. In some embodiments, the subject can be in need thereof. Further disclosed herein are pharmaceutical compositions for use in the methods disclosed herein. In some embodiments, the pharmaceutical composition can comprise the BAFF inhibitor or a salt thereof; and the immune checkpoint inhibitor or a salt thereof. In some embodiments, the pharmaceutical composition can further comprise a pharmaceutically acceptable excipient. In some embodiments, the pharmaceutical composition can be in unit dose form. Disclosed herein are bispecific antibodies. In some embodiments, the bispecific antibody can comprise a first domain, wherein the first domain binds BAFF, BAFF-R or a portion thereof; and a second domain, wherein the second domain binds PD-1, PD-L1 or a portion thereof. In some embodiments, the first domain can comprise a heavy chain variable region, light chain variable region, light chain or heavy chain of tabalumab, atacicept, RCT-18, BAFF/IL-17 bispecific antibody, ardenermin, briobacept, blisibimod, belimumab, MEDI-700, NOV-5, rGel/BlyS or a combination thereof. In some embodiments, the second domain can comprise a heavy chain variable region, light chain variable region, light chain or heavy chain of nivolumab, docetaxel, pembrolizumab, pidilizumab, BGB-A31, MEDI0680, AMP-224, MEDI0680, PDR001, Cemiplimab or a combination thereof. In some embodiments, disclosed herein are the bispecific antibody and a pharmaceutically acceptable carrier. In some embodiments, the compositions disclosed herein can be used in a method disclosed herein.

In an aspect, the present disclosure provides a method of treating a subject in need thereof. The method may comprise: administering a therapeutically effective amount of at least one B-cell activating factor (BAFF) inhibitor and/or at least one B-cell activating factor receptor (BAFF-R) inhibitor, or a salt thereof; and administering a therapeutically effective amount of at least one checkpoint inhibitor or a salt thereof. The subject may have been diagnosed with a cancer or a tumor. The method may be a method of treating a cancer or a tumor in the subject. The subject may have not previously undergone treatment for the cancer or the tumor. The subject may have previously or may be currently undergoing treatment for the cancer or the tumor. The subject may have previously undergone treatment with surgery or radiation, and the subject may remain in remission. The subject may have previously undergone treatment with surgery, radiation, an anticancer agent, and any combination thereof, and the cancer or the tumor may have been at least partially refractive to the treatment. In some embodiments, a subject may have been previously treated with surgery, radiation therapy, an anticancer agent, and any combination thereof, and the cancer or the tumor may have re-occurred in the subject following a period of remission, wherein the period of remission may have been at least about: 1 month, 2 months, 3 months, 6 months, 1 year, 2 years, 3 years, 4 years, 5 years, 6 years, 7 years, 8 years, 9 years, or 10 years. The subject may have been previously treated with a checkpoint inhibitor or a salt thereof, and the cancer or the tumor may have been at least partially refractive to the checkpoint inhibitor or a salt thereof. The subject may have been previously treated with a checkpoint inhibitor or a salt thereof, and the cancer or the tumor may have re-occurred in the subject following a period of remission, wherein a period of the remission may have been at least about: 1 month, 2 months, 3 months, 6 months, 1 year, 2 years, 3 years, 4 years, 5 years, 6 years, 7 years, 8 years, 9 years, or 10 years. The checkpoint inhibitor or a salt thereof may be administered sequentially with the BAFF inhibitor and/or BAFF-R inhibitor or a salt thereof. In some embodiments, the checkpoint inhibitor or a salt thereof may be administered concurrently with the BAFF inhibitor and/or BAFF-R inhibitor or a salt thereof. The checkpoint inhibitor may comprise an agent that binds to anti-programmed cell death protein 1 (PD-1), anti-programmed death ligand 1 (PD-L1), anti-programmed death ligand 2 (PD-L2), cytotoxic T-lymphocyte-associated protein 4 (CTLA4), cluster of differentiation 276 (B7-H3), V-set domain-containing T-cell activation inhibitor 1 (B7-H4), T-cell immunoglobulin and mucin-domain containing-3 (TIM-3), lymphocyte-activation gene 3 (LAG3), Indoleamine-pyrrole 2,3-dioxygenase (IDO), a salt of any one thereof, or any combination thereof. The checkpoint inhibitor may be selected from the group consisting of an agent that binds to anti-programmed cell death protein 1 (PD-1), anti-programmed death ligand 1 (PD-L1), anti-programmed death ligand 2 (PD-L2), cytotoxic T-lymphocyte-associated protein 4 (CTLA4), cluster of differentiation 276 (B7-H3), V-set domain-containing T-cell activation inhibitor 1 (B7-H4), T-cell immunoglobulin and mucin-domain containing-3 (TIM-3), lymphocyte-activation gene 3 (LAG3), Indoleamine-pyrrole 2,3-dioxygenase (IDO), a salt of any one thereof, and any combination thereof. The checkpoint inhibitor or a salt thereof may be a PD-1 inhibitor or a salt thereof. In some embodiments, the PD-1 inhibitor or a salt thereof may comprise nivolumab, pembrolizumab, pidilizumab, BGB-A31, MEDI0680 (AMP-514), a salt of any one thereof, or any combination thereof. The PD-1 inhibitor or a salt thereof may be selected from the group consisting of nivolumab, pembrolizumab, pidilizumab, BGB-A31, MEDI0680 (AMP-514), a salt of any one thereof, and any combination thereof. The PD-1 inhibitor may be nivolumab or a salt thereof. The PD-1 inhibitor may be pembrolizumab or a salt thereof. The PD-1 inhibitor may be pidilizumab or a salt thereof. The PD-1 inhibitor may be BGB-A31 or a salt thereof. The PD-1 inhibitor may be MEDI680 (AMP-514) or a salt thereof. The checkpoint inhibitor or a salt thereof may be a PD-L1 inhibitor or a salt thereof. The PD-L1 inhibitor or a salt thereof may comprise atezolizumab, avelumab, durvalumab, MDX-1105, MSB0010718C, a salt of any one thereof, or any combination thereof. The PD-L1 inhibitor or a salt thereof may be selected from the group consisting of atezolizumab, avelumab, durvalumab, MDX-1105, MSB0010718C, a salt of any one thereof, and any combination thereof. The PD-L1 inhibitor may be atezolizumab or a salt thereof. The PD-L1 inhibitor may be avelumab or a salt thereof. The PD-L1 inhibitor may be durvalumab or a salt thereof. In some embodiments, the PD-L1 inhibitor may be MDX-1105 or a salt thereof. In some embodiments, the PD-L1 inhibitor may be MSB0010718C or a salt thereof. In some embodiments, the checkpoint inhibitor may be a CTLA4 inhibitor or a salt thereof. The CTLA4 inhibitor or a salt thereof may comprise ipilimumab, tremelimumab, AGEN1884, a salt of any one thereof, or any combination thereof. The CTLA4 inhibitor or a salt thereof may be selected from the group consisting of ipilimumab, tremelimumab, AGEN1884, a salt of any one thereof, and any combination thereof. The CTLA4 inhibitor may be ipilimumab or a salt thereof. The CTLA4 inhibitor may be tremelimumab or a salt thereof. The CTLA4 inhibitor may be AGEN1884 or a salt thereof. In some embodiments, the checkpoint inhibitor or a salt thereof may be a LAG3 inhibitor or a salt thereof. The LAG3 inhibitor may be BMS-986016 or a salt thereof. The checkpoint inhibitor or a salt thereof may be a TIM3 inhibitor or a salt thereof. The TIM3 inhibitor or a salt thereof may comprise MBG453, TSR-022, a salt of any one thereof, or any combination thereof. The TIM3 inhibitor or a salt thereof may be selected from the group consisting of MBG453, TSR-022, a salt of any one thereof, and any combination thereof. The TIM3 inhibitor may be MBG453 or a salt thereof. The TIM3 inhibitor may be TSR-022 or a salt thereof. In some embodiments, the BAFF inhibitor may be an antibody, antigen binding fragment, a bispecific antibody, or a recombinant fusion protein. The BAFF inhibitor antibody may be human, humanized, chimeric, composite, polyclonal or monoclonal. The BAFF antibody or antigen binding fragment may block interaction of molecules to BAFF. The BAFF inhibitor may comprise tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, or any combination thereof. The BAFF inhibitor may be selected from the group consisting of tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, and any combination thereof. In some embodiments, the BAFF inhibitor may comprise MEDI-700, NOV-5, rGel/BLyS, a salt of any one thereof, or any combination thereof. The BAFF inhibitor may be selected from the group consisting of MEDI-700, NOV-5, rGel/BLyS, a salt of any one thereof, and any combination thereof. The BAFF inhibitor may be tabalumab or a salt thereof. The BAFF inhibitor may be blisibimod or a salt thereof. The BAFF inhibitor may be belimumab or a salt thereof. The BAFF inhibitor may be atacicept or a salt thereof. In an aspect, the present disclosure provides a kit comprising a composition described herein. In some embodiments, the kit may contain instructions for use.

In an aspect, the present disclosure provides a method of treating a subject in need thereof. The method may comprise administering a therapeutically effective amount of at least one BAFF and/or BAFF-R inhibitor or a salt thereof to the subject; and administering at least one immune agonist agent or a salt thereof to the subject. The immune agonist agent or a salt thereof may comprise an agent that binds to glucocorticoid-induced tumor necrosis factor receptor-related protein (GITR), cluster of differentiation 134 (OX40), cluster of differentiation 137 (CD137), cluster of differentiation 40 (CD40), Toll-like receptor (TLR), a salt of any one thereof, or any combination thereof. The immune agonist agent or a salt thereof may be selected from the group consisting of an agent that binds to glucocorticoid-induced tumor necrosis factor receptor-related protein (GITR), cluster of differentiation 134 (OX40), cluster of differentiation 137 (CD137), cluster of differentiation 40 (CD40), Toll-like receptor (TLR), a salt of any one thereof, and any combination thereof. The immune agonist agent may be a GITR agonist or a salt thereof. The GITR agonist or a salt thereof may comprise an agent that binds to TRX518, GWN323, MEDI1873, INCAGN01876, a salt of any one thereof, or any combination thereof. The GITR agonist or a salt thereof may be selected from the group consisting of an agent that binds to TRX518, GWN323, MEDI1873, INCAGN01876, a salt of any one thereof, and any combination thereof. The GITR agonist may be TRX518 or a salt thereof. The GITR agonist may be GWN323 or a salt thereof. The GITR agonist may be MEDI1873 or a salt thereof. The GITR agonist may be INCAGN01876 or a salt thereof. The immune agonist agent may be an OX40 agonist or a salt thereof. In some embodiments, the OX40 agonist may comprise GSK3174998, PF-04518600, MEDI6469, INCAGN01949, a salt of any one thereof, or any combination thereof. The OX40 agonist may be selected from the group consisting of GSK3174998, PF-04518600, MEDI6469, INCAGN01949, a salt of any one thereof, and any combination thereof. The OX40 agonist may be GSK3174998 or a salt thereof. The OX40 agonist may be PF-04518600 or a salt thereof. The OX40 agonist may be MEDI6469 or a salt thereof. The OX40 agonist may be INCAGN01949 or a salt thereof. In some embodiments, the immune agonist agent may be a cluster of differentiation 137 (4-1BB) agonist or a salt thereof. The 4-1BB agonist may be urelumab, utomilumab, or a salt of any one thereof. The 4-1BB agonist may be isurelumab or a salt thereof. The 4-1BB agonist may be isutomilumab or a salt thereof. The immune agonist agent may be a CD40 agonist or a salt thereof. The CD40 agonist may be APX005M, CP870893, or a salt thereof. The CD40 agonist may be APX005M or a salt thereof. The CD40 agonist may be CP870893 or a salt thereof. The immune agonist agent may be a TLR agonist or a salt thereof. The TLR agonist may comprise an agent that binds to TLR-1, TLR-2, TLR-3, TLR-4, TLR-5, TLR-6, TLR-7, TLR-8, TLR-9, TLR-13, a salt or any one thereof, or any combination thereof. The TLR agonist may be selected from the group consisting of an agent that binds to TLR-1, TLR-2, TLR-3, TLR-4, TLR-5, TLR-6, TLR-7, TLR-8, TLR-9, TLR-13, a salt or any one thereof, and any combination thereof. In some embodiments, the BAFF inhibitor may be an antibody, antigen binding fragment, a bispecific antibody, or a recombinant fusion protein. The BAFF inhibitor antibody may be human, humanized, chimeric, composite, polyclonal or monoclonal. The BAFF antibody or antigen binding fragment may block interaction of molecules to BAFF. The BAFF inhibitor may comprise tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, or any combination thereof. The BAFF inhibitor may be selected from the group consisting of tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, and any combination thereof. In some embodiments, the BAFF inhibitor may comprise MEDI-700, NOV-5, rGel/BLyS, a salt of any one thereof, or any combination thereof. The BAFF inhibitor may be selected from the group consisting of MEDI-700, NOV-5, rGel/BLyS, a salt of any one thereof, and any combination thereof. The BAFF inhibitor may be tabalumab or a salt thereof. The BAFF inhibitor may be blisibimod or a salt thereof. The BAFF inhibitor may be belimumab or a salt thereof. The BAFF inhibitor may be atacicept or a salt thereof. In an aspect, the present disclosure provides a kit comprising a composition described herein. In some embodiments, the kit may contain instructions for use.

In an aspect, the present disclosure provides a method of treating a subject in need thereof. The method may comprise: administering a therapeutically effective amount of at least one BAFF and/or BAFF-R inhibitor or a salt thereof; and administering at least one vaccine agent or a salt thereof. The vaccine agent may comprise MAGE-3, NY-ESO-1, TRAG-3, p53, at least one or more α-actinin-4 and malic enzymes, carcinoembryonic antigen, HER2, MUC1, survivin, WT-1, PRAME, Survivin-2b, Bacillus Calmette-Guerin, MVAX, at least one or more heat shock proteins, keyhole limpet hemocyanin, interleukin-2, QS21, montanide ISA-51, granulocyte monocyte-colony stimulating factor, GVAX, GI-4000, CDX-1307, IMA910, TroVAX, CRS-207, CA-9, a salt of any one thereof, or any combination thereof. The vaccine agent may be selected from the group consisting of MAGE-3, NY-ESO-1, TRAG-3, p53, at least one or more α-actinin-4 and malic enzymes, carcinoembryonic antigen, HER2, MUC1, survivin, WT-1, PRAME, Survivin-2b, Bacillus Calmette-Guerin, MVAX, at least one or more heat shock proteins, keyhole limpet hemocyanin, interleukin-2, QS21, montanide ISA-51, granulocyte monocyte-colony stimulating factor, GVAX, GI-4000, CDX-1307, IMA910, TroVAX, CRS-207, CA-9, a salt of any one thereof, and any combination thereof. In some embodiments, the BAFF inhibitor may be an antibody, antigen binding fragment, a bispecific antibody, or a recombinant fusion protein. The BAFF inhibitor antibody may be human, humanized, chimeric, composite, polyclonal or monoclonal. The BAFF antibody or antigen binding fragment may block interaction of molecules to BAFF. The BAFF inhibitor may comprise tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, or any combination thereof. The BAFF inhibitor may be selected from the group consisting of tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, and any combination thereof. In some embodiments, the BAFF inhibitor may comprise MEDI-700, NOV-5, rGel/BLyS, a salt of any one thereof, or any combination thereof. The BAFF inhibitor may be selected from the group consisting of MEDI-700, NOV-5, rGel/BLyS, a salt of any one thereof, and any combination thereof. The BAFF inhibitor may be tabalumab or a salt thereof. The BAFF inhibitor may be blisibimod or a salt thereof. The BAFF inhibitor may be belimumab or a salt thereof. The BAFF inhibitor may be atacicept or a salt thereof. In an aspect, the present disclosure provides a kit comprising a composition described herein. In some embodiments, the kit may contain instructions for use.

In an aspect, the present disclosure provides a method of treating a subject in need thereof. The method may comprise: administering a therapeutically effective amount of at least one BAFF and/or BAFF-R inhibitor or a salt thereof; and administering at least one oncolytic viral based agent or a salt thereof. The oncolytic viral based agent may comprise enadenotucirev, talimogene laherparepvec, reolysin, CG0070, Pexastimogene devacirepvec, cavatak, oncolytic vesicular stomatitis virus, ONCOS-102, a salt of any one thereof, or any combination thereof. The oncolytic viral based agent may be selected from the group consisting of enadenotucirev, talimogene laherparepvec, reolysin, CG0070, Pexastimogene devacirepvec, cavatak, oncolytic vesicular stomatitis virus, ONCOS-102, a salt of any one thereof, and any combination thereof. In some embodiments, the BAFF inhibitor may be an antibody, antigen binding fragment, a bispecific antibody, or a recombinant fusion protein. The BAFF inhibitor antibody may be human, humanized, chimeric, composite, polyclonal or monoclonal. The BAFF antibody or antigen binding fragment may block interaction of molecules to BAFF. The BAFF inhibitor may comprise tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, or any combination thereof. The BAFF inhibitor may be selected from the group consisting of tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, and any combination thereof. In some embodiments, the BAFF inhibitor may comprise MEDI-700, NOV-5, rGel/BLyS, a salt of any one thereof, or any combination thereof. The BAFF inhibitor may be selected from the group consisting of MEDI-700, NOV-5, rGel/BLyS, a salt of any one thereof, and any combination thereof. The BAFF inhibitor may be tabalumab or a salt thereof. The BAFF inhibitor may be blisibimod or a salt thereof. The BAFF inhibitor may be belimumab or a salt thereof. The BAFF inhibitor may be atacicept or a salt thereof. In an aspect, the present disclosure provides a kit comprising a composition described herein. In some embodiments, the kit may contain instructions for use.

In an aspect, the present disclosure provides a method of treating a subject in need thereof. The method may comprise: administering a therapeutically effective amount of at least one BAFF and/or BAFF-R inhibitor or a salt thereof; and administering at least one cell-based therapy, any cell derivative thereof, or a salt of any one thereof. The cell-based therapy may comprise at least one or more autologous lymphocytes, at least one or more genetically engineered autologous lymphocytes, at least one or more chimeric antigen receptor cells, at least one or more chimeric antigen receptor T-cells, at least one or more dendritic cell based vaccines, a salt of any one thereof, or any combination thereof. The cell-based therapy may be selected from the group consisting of: at least one or more autologous lymphocytes, at least one or more genetically engineered autologous lymphocytes, at least one or more chimeric antigen receptor cells, at least one or more chimeric antigen receptor T-cells, at least one or more dendritic cell based vaccines, a salt of any one thereof, and any combination thereof. In some embodiments, the BAFF inhibitor may be an antibody, antigen binding fragment, a bispecific antibody, or a recombinant fusion protein. The BAFF inhibitor antibody may be human, humanized, chimeric, composite, polyclonal or monoclonal. The BAFF antibody or antigen binding fragment may block interaction of molecules to BAFF. The BAFF inhibitor may comprise tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, or any combination thereof. The BAFF inhibitor may be selected from the group consisting of tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, and any combination thereof. In some embodiments, the BAFF inhibitor may comprise MEDI-700, NOV-5, rGel/BLyS, a salt of any one thereof, or any combination thereof. The BAFF inhibitor may be selected from the group consisting of MEDI-700, NOV-5, rGel/BLyS, a salt of any one thereof, and any combination thereof. The BAFF inhibitor may be tabalumab or a salt thereof. The BAFF inhibitor may be blisibimod or a salt thereof. The BAFF inhibitor may be belimumab or a salt thereof. The BAFF inhibitor may be atacicept or a salt thereof. In an aspect, the present disclosure provides a kit comprising a composition described herein. In some embodiments, the kit may contain instructions for use.

In an aspect, the present disclosure provides a method of treating a subject in need thereof. The method may comprise: administering a therapeutically effective amount of at least one BAFF and/or BAFF-R inhibitor or a salt thereof, and administering at least one chemotherapeutic agent or a salt thereof. The chemotherapeutic agent may be an alkylating agent, an antimetabolite agent, a plant alkaloid agent, an antitumor antibiotic, a salt of any one thereof, or any combination thereof. In some embodiments, the BAFF inhibitor may be an antibody, antigen binding fragment, a bispecific antibody, or a recombinant fusion protein. The BAFF inhibitor antibody may be human, humanized, chimeric, composite, polyclonal or monoclonal. The BAFF antibody or antigen binding fragment may block interaction of molecules to BAFF. The BAFF inhibitor may comprise tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, or any combination thereof. The BAFF inhibitor may be selected from the group consisting of tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, and any combination thereof. In some embodiments, the BAFF inhibitor may comprise MEDI-700, NOV-5, rGel/BLyS, a salt of any one thereof, or any combination thereof. The BAFF inhibitor may be selected from the group consisting of MEDI-700, NOV-5, rGel/BLyS, a salt of any one thereof, and any combination thereof. The BAFF inhibitor may be tabalumab or a salt thereof. The BAFF inhibitor may be blisibimod or a salt thereof. The BAFF inhibitor may be belimumab or a salt thereof. The BAFF inhibitor may be atacicept or a salt thereof. In an aspect, the present disclosure provides a kit comprising a composition described herein. In some embodiments, the kit may contain instructions for use.

In an aspect, the present disclosure provides a method of treating a subject in need thereof. The method may comprise: administering a therapeutically effective amount of at least one BAFF and/or BAFF-R inhibitor or a salt thereof; and administering at least one targeted therapeutic agent or a salt thereof. The targeted therapeutic agent may be an agent that inhibits signal transduction, angiogenesis, hormone expression, or any combination thereof. The BAFF inhibitor and/or BAFF-R inhibitor may prevent binding of BAFF or a BAFF-induced ligand to a BAFF-R, may be a partial antagonist of a BAFF may be a partial agonist of a BAFF, may be a competitive antagonist of a BAFF-R, may be a non-competitive antagonist of a BAFF-R receptor, or any combination thereof. In some embodiments, the BAFF inhibitor may be an antibody, antigen binding fragment, a bispecific antibody, or a recombinant fusion protein. The BAFF inhibitor antibody may be human, humanized, chimeric, composite, polyclonal or monoclonal. The BAFF antibody or antigen binding fragment may block interaction of molecules to BAFF. The BAFF inhibitor may comprise tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, or any combination thereof. The BAFF inhibitor may be selected from the group consisting of tabalumab, atacicept, RCT-18, BAFF/IL-17 bispecific antibody, ardenermin, briobacept, blisibimod, belimumab, a transmembrane activator and calcium-modulator and cyclophilin ligand interactorcept, a salt of any one thereof, and any combination thereof. In some embodiments, the BAFF inhibitor may comprise MEDI-700, NOV-5, rGel/BLyS, a salt of any one thereof, or any combination thereof. The BAFF inhibitor may be selected from the group consisting of MEDI-700, NOV-5, rGel/BLyS, a salt of any one thereof, and any combination thereof. The BAFF inhibitor may be tabalumab or a salt thereof. The BAFF inhibitor may be blisibimod or a salt thereof. The BAFF inhibitor may be belimumab or a salt thereof. The BAFF inhibitor may be atacicept or a salt thereof. In an aspect, the present disclosure provides a kit comprising a composition described herein. In some embodiments, the kit may contain instructions for use.

In an aspect, the present disclosure provides a method of treating a subject in need thereof. The method may comprise: administering a therapeutically effective amount of an immunotherapeutic agent or a salt thereof, wherein the immunotherapeutic agent or a salt thereof may inhibit a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof, wherein the immunotherapeutic agent may be co-administered with a checkpoint inhibitor. The subject may have been diagnosed with a cancer or a tumor. The method may be a method of treating a cancer or a tumor in the subject. The subject may have previously or may be currently undergoing treatment for the cancer or the tumor. The subject may have been previously treated with a checkpoint inhibitor, and the cancer or the tumor may have been at least partially refractive to the checkpoint inhibitor. In some embodiments, the method may further comprise administering a therapeutically effective amount of the checkpoint inhibitor. The checkpoint inhibitor may be a salt. The checkpoint inhibitor may be selected from the group consisting of an anti-programmed cell death protein 1 agent, an anti-programmed death ligand 1 agent, an anti-programmed death ligand 2 agent, a salt of any of these, and any combination thereof. The method may further comprise administering the anti-programmed cell death protein 1 agent or a salt thereof. The anti-programmed cell death protein 1 agent or a salt thereof may comprise docetaxel, nivolumab, pembrolizumab, a salt of any of these, or any combination thereof. The anti-programmed cell death protein 1 agent or a salt thereof may be selected from the group consisting of docetaxel, nivolumab, pembrolizumab, a salt of any of these, and any combination thereof. The anti-programmed cell death protein 1 agent or a salt thereof may be pembrolizumab. The method may comprise administering the anti-programmed death ligand 1 agent or a salt thereof to the subject. The anti-programmed death ligand 1 agent or a salt thereof may comprise atezolizumab, avelumab, durvalumab, a salt of any of these, or any combination thereof. The anti-programmed death ligand 1 agent or a salt thereof may be selected from the group consisting of atezolizumab, avelumab, durvalumab, a salt of any of these, and any combination thereof. In some embodiments, the immunotherapeutic agent or a salt thereof at least partially may prevent binding of a ligand to a B-cell activating factor receptor; may be a partial antagonist of a B-cell activating factor receptor; may be a partial agonist of a B-cell activating factor receptor; may be a competitive antagonist of a B-cell activating factor receptor; may be a non-competitive antagonist of a B-cell activating factor receptor; or any combination thereof. The immunotherapeutic agent or a salt thereof at least partially may prevent ligand binding to a transmembrane activator and calcium-modulator and cyclophilin ligand interactor receptor; may be a partial antagonist of a transmembrane activator and calcium-modulator and cyclophilin ligand interactor receptor; may be a partial agonist of a transmembrane activator and calcium-modulator and cyclophilin ligand interactor receptor; or any combination thereof. In some embodiments, the immunotherapeutic agent or a salt thereof at least partially may prevent ligand binding to a B-cell maturation antigen receptor; may be a partial antagonist of a B-cell maturation antigen receptor; may be a partial agonist of a B-cell maturation antigen receptor; or any combination thereof. The immunotherapeutic agent may be an antibody or a salt thereof. The immunotherapeutic agent may be a human or humanized monoclonal antibody or a salt thereof. The immunotherapeutic agent or a salt thereof may comprise ardenermin, briobacept, blisibimod, belimumab, a transmembrane activator and calcium-modulator and cyclophilin ligand interactorcept, tabalumab, atacicept, RCT-18, a salt of any of these, or any combination thereof. The immunotherapeutic agent or a salt thereof may be selected from the group consisting of ardenermin, briobacept, blisibimod, belimumab, a transmembrane activator and calcium-modulator and cyclophilin ligand interactorcept, tabalumab, atacicept, RCT-18, a salt of any of these, and any combination thereof. The immunotherapeutic agent or a salt thereof may comprise MEDI-700, NOV-5, rGel/BLyS, a salt of any of these, or any combination thereof. The immunotherapeutic agent or a salt thereof may be selected from the group consisting of MEDI-700, NOV-5, rGel/BLyS, a salt of any of these, and any combination thereof. In some embodiments, the immunotherapeutic agent may be tabalumab or a salt thereof. The immunotherapeutic agent may be blisibimod or a salt thereof. The immunotherapeutic agent may be belimumab or a salt thereof. The immunotherapeutic agent may be atacicept or a salt thereof. The method may further comprise, before the administering: determining a count of cluster of differentiation 8 (CD8) protein, and at least one selected from the group consisting of: cluster of differentiation 19 (CD19) protein, cluster of differentiation 20 protein (CD20), cluster of differentiation 138 (CD138) protein, B-cell activating factor protein, B-cell activating factor receptor protein, and paired box Pax-5 (PAX5) protein, in a cancer or a tumor or a cancer sample or a tumor sample of a subject. In some embodiments, the at least one of: CD19, CD20, CD138, B-cell activating factor, B-cell activating factor-R, and PAX5 may be present in the cancer or the tumor or the cancer sample or the tumor sample of the subject. In some embodiments, the at least one of: CD19, CD20, CD138, B-cell activating factor, B-cell activating factor-R, and PAX5 may be individually present in the cancer or the tumor or the cancer sample or the tumor sample of the subject in an amount ranging from 1 protein to at least about 10,000,000 proteins. The method may further comprise monitoring the treatment after the administering. In some embodiments, the treatment may maintain at least partial remission of the cancer or the tumor, wherein a period of the remission may have been at least about: 1 month, 2 months, 3 months, 6 months, 1 year, 2 years, 3 years, 4 years, 5 years, 6 years, 7 years, 8 years, 9 years, or 10 years. The treatment may comprise, after administering, delaying progression of the cancer or the tumor in the subject. The treatment may comprise, after administering, regression of the cancer or the tumor. Regression may be a reduction in mass of the cancer or the tumor, a reduction in volume of the cancer or the tumor, or both. The treatment may comprise prolonging the subject's life. The subject may have previously shown at least partial refraction to a monotherapy for the cancer or the tumor. The subject may have shown refraction to a monotherapy for the cancer or the tumor. The cancer may have metastasized from a first location of the subject to a second location of the subject. In some embodiments, the cancer at the first location of the body may be less than fully responsive to the monotherapy, and wherein the cancer at the second location of the body may be responsive to the treatment. The checkpoint inhibitor and the immunotherapeutic agent may be synergistic. The synergy on the cancer or the tumor may be at least 10% more than an additive effect. A heatmap of a cancer sample or a tumor sample of the subject may show a profile substantially similar to that of FIG. 8. In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof may be administered concurrently. The anti-programmed cell death protein 1 agent, the anti-programmed death ligand 1 agent, the anti-programmed death ligand 2 agent, or a salt thereof or any combination thereof, and the immunotherapeutic agent or a salt thereof may be administered sequentially. The anti-programmed cell death protein 1 agent, the anti-programmed death ligand 1 agent, the anti-programmed death ligand 2 agent, or a salt thereof or any combination thereof, and the immunotherapeutic agent or a salt thereof may be administered in different formulations within a same treatment schedule. In some embodiments, the anti-programmed cell death protein 1 agent, the anti-programmed death ligand 1 agent, the anti-programmed death ligand 2 agent, or a salt thereof or any combination thereof, may be administered at least once a week. The anti-programmed cell death protein 1 agent, the anti-programmed death ligand 1 agent, the anti-programmed death ligand 2 agent, or a salt thereof or any combination thereof, may be administered at least once a day. The anti-programmed cell death protein 1 agent, the anti-programmed death ligand 1 agent, the anti-programmed death ligand 2 agent, or a salt thereof or any combination thereof, may be administered at least once during a treatment schedule. In some embodiments, the immunotherapeutic agent or a salt thereof may be administered at least once a week. The immunotherapeutic agent or a salt thereof may be administered at least once a day. The immunotherapeutic agent or a salt thereof may be administered at least once during a treatment schedule. The immunotherapeutic agent or a salt thereof may be administered in an amount from about 0.1 mg to about 1,000 mg per kg body weight. In some embodiments, the immunotherapeutic agent or a salt thereof may be administered in an amount from about 0.1 mg to about 100 mg per kg body weight. The immunotherapeutic agent or a salt thereof may be administered in an amount from about 1 mg to about 50 mg per kg body weight. The immunotherapeutic agent or a salt thereof may be administered in a pharmaceutical composition. The anti-programmed cell death protein 1 agent, the anti-programmed death ligand 1 agent, the anti-programmed death ligand 2 agent, or a salt thereof or any combination thereof, may be administered in an amount from about 0.1 mg to about 1,000 mg per kg body weight. The anti-programmed cell death protein 1 agent, the anti-programmed death ligand 1 agent, the anti-programmed death ligand 2 agent, or a salt thereof or any combination thereof, may be administered in an amount from about 1 mg to about 100 mg per kg body weight. The anti-programmed cell death protein 1 agent, the anti-programmed death ligand 1 agent, the anti-programmed death ligand 2 agent, or a salt of any one thereof, or any combination thereof, may be individually present as a pharmaceutical formulation. In some embodiments, the pharmaceutical formulation may be in unit dose form. The pharmaceutical formulation may further comprise a pharmaceutically-acceptable excipient. At least one of the anti-programmed cell death protein 1 agent, the anti-programmed death ligand 1 agent, the anti-programmed death ligand 2 agent, or a salt of any one thereof, or any combination thereof, and the immunotherapeutic agent or salt thereof may be isolated and purified. The administration may be oral. The administration may be topical, intravenous, intramuscular, or spinal. The administration may be administered directly to the cancer or the tumor. The administration may be an administration at a location different from the cancer or the tumor. The subject may have been previously diagnosed with the cancer or the tumor. The cancer or the tumor may be a solid tumor or a solid cancer. The cancer or the tumor may be a liquid cancer or a liquid tumor. The cancer or the tumor may be malignant. The cancer or the tumor may be present in an organ. The tumor or the cancer may be present in at least one of: blood, lymph, cerebral spinal fluid. The cancer or the tumor may be located in a head or neck region, abdominal region, a upper limb, a lower limb, skin, blood, digestive tract, germ cell, or nervous system. The method may comprise treating the cancer, wherein the cancer may be prostate cancer, testicular cancer, breast cancer, brain cancer, pancreatic cancer, colon cancer, thyroid cancer, stomach cancer, lung cancer, melanoma, multiple myeloma, Hodgkin's lymphoma, ovarian cancer, or any combination thereof. In an aspect, the present disclosure provides a kit comprising a composition described herein. In some embodiments, the kit may contain instructions for use.

In an aspect, the present disclosure provides a composition. The composition may comprise an anti-programmed cell death protein 1 agent, an anti-programmed death ligand 1 agent, an anti-programmed death ligand 2 agent, or a salt of any one thereof, or any combination thereof, and an immunotherapeutic agent or a salt thereof. The composition may be a pharmaceutical composition. The pharmaceutical composition may be in unit dose form. The immunotherapeutic agent or a salt thereof may be an antibody or a salt thereof. The immunotherapeutic agent or a salt thereof may be a human or humanized monoclonal antibody or a salt thereof. The immunotherapeutic agent or a salt thereof may comprise ardenermin, briobacept, blisibimod, belimumab, a transmembrane activator and calcium-modulator and cyclophilin ligand interactorcept, MEDI-0700, NOV-5, rGel/BLyS, tabalumab, atacicept, RCT-18, a salt of any of these, or any combination thereof. The immunotherapeutic agent or a salt thereof may be selected from the group consisting of ardenermin, briobacept, blisibimod, belimumab, a transmembrane activator and calcium-modulator and cyclophilin ligand interactorcept, MEDI-0700, NOV-5, rGel/BLyS, tabalumab, atacicept, RCT-18, a salt of any of these, and any combination thereof. The immunotherapeutic agent or a salt thereof may be tabalumab or a salt thereof. The method may further comprise a pharmaceutically acceptable excipient. In some embodiments, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof may be independently administered in an amount from about 0.1 mg to about 10 g. The anti-programmed cell death protein 1 agent, the anti-programmed death ligand 1 agent, the anti-programmed death ligand 2 agent, or a salt of any one thereof, or any combination thereof, and the immunotherapeutic agent or a salt thereof may be independently 0.001% to 99% by weight of the composition. The anti-programmed cell death protein 1 agent, the anti-programmed death ligand 1 agent, the anti-programmed death ligand 2 agent, or a salt of any one thereof, or any combination thereof, and the immunotherapeutic agent or a salt thereof may be independently administered in an amount of about 0.1 mg to about 100 mg per kg body weight. The composition may be in a form of a tablet, a capsule, a gel, or a liquid formulation. In an aspect, the present disclosure provides a kit comprising a composition described herein. In some embodiments, the kit may contain instructions for use.

In an aspect, the present disclosure provides a method of making a kit described herein. The method may comprise contacting or combining the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof. The composition may be in a form of a tablet, a capsule, a gel, or a liquid formulation. The method may comprise contacting or combining the anti-programmed cell death protein 1 agent, the anti-programmed death ligand 1 agent, the anti-programmed death ligand 2 agent, or a salt thereof or any combination thereof, and the immunotherapeutic agent or a salt thereof.

In an aspect, the present disclosure provides a method of selecting a therapeutic regimen. The method may comprise: determining the presence or absence of cluster of differentiation 8 (CD8) protein, and at least one of: B-cell activating factor (BAFF) protein, B-cell activating factor receptor (BAFF-R) protein, and paired box Pax-5 (PAX5) protein in a cancer or a tumor of a subject or a cancer sample or a tumor sample of the subject; if CD8 protein and at least one of B-cell activating factor, B-cell activating factor-R, and PAX5 may be present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof; if CD8 protein and at least one of B-cell activating factor, B-cell activating factor-R, and PAX5 may be not present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof. The method may further comprise determining whether cluster of differentiation 19 (CD19) protein, cluster of differentiation 20 (CD20) protein, cluster of differentiation 138 (CD138) protein, or any combination thereof may be present. The method may further comprise treating the subject. The treating may comprise: administering a therapeutically effective amount of an immunotherapeutic agent or a salt thereof, wherein the immunotherapeutic agent or a salt thereof may inhibit a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof, wherein the immunotherapeutic agent may be co-administered with a checkpoint inhibitor. In some embodiments, the subject may have been diagnosed with a cancer or a tumor. The subject may have previously or may be currently undergoing treatment for the cancer or the tumor. The method may comprise administering a therapeutically effective amount of the checkpoint inhibitor. The checkpoint inhibitor may be selected from the group consisting of an anti-programmed cell death protein 1 agent, an anti-programmed death ligand 1 agent, an anti-programmed death ligand 2 agent, a salt of any of these, and any combination thereof. The method may comprise administering the anti-programmed cell death protein 1 agent of a salt thereof. In some embodiments, the anti-programmed cell death protein 1 agent or a salt thereof may comprise docetaxel, nivolumab, pembrolizumab, a salt of any of these, or any combination thereof. The anti-programmed cell death protein 1 agent or a salt thereof may be selected from the group consisting of docetaxel, nivolumab, pembrolizumab, a salt of any of these, and any combination thereof. The anti-programmed cell death protein 1 agent or a salt thereof may be pembrolizumab. The immunotherapeutic agent may be tabalumab or a salt thereof. The immunotherapeutic agent may be belimumab or a salt thereof. The immunotherapeutic agent may be atacicept or a salt thereof. The immunotherapeutic agent may be blisibimod or a salt thereof. In some embodiments, the cancer or the tumor may be prostate cancer, testicular cancer, breast cancer, brain cancer, pancreatic cancer, colon cancer, thyroid cancer, stomach cancer, lung cancer, ovarian cancer, or any combination thereof. The cancer or the tumor may contact a blood vessel. The cancer or the tumor may be in the interior of a blood vessel.

In an aspect, the present disclosure provides a method of selecting a therapeutic regimen. The method may comprise: determining a count of B cells, T cells, and plasma cells in a cancer or a tumor of a subject or a cancer sample or a tumor sample of the subject; if a count of T cells in the cancer or the tumor or the cancer sample or the tumor sample may be greater than 500 cells/mm2 and a count of B cells may be less than <150 cells/mm2, selecting a therapeutic regimen comprising an anti-programmed cell death protein 1 agent, an anti-programmed death ligand 1 agent, an anti-programmed death ligand 2 agent, or a salt thereof or any combination thereof, if a count of T cells in the cancer or the tumor or the cancer sample or the tumor sample may be greater than 300 cells/mm2 and a count of B cells, a count of plasma cells, or a combination of B cells and plasma cells may be greater than 100 cells/mm2, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof. In some embodiments, the method may further comprise conveying a result via a communication medium.

In an aspect, the present disclosure provides a method of treating or maintaining remission of a cancer or a tumor in a subject. The method may comprise: administering an anti-programmed cell death protein 1 agent, an anti-programmed death ligand 1 agent, an anti-programmed death ligand 2 agent, or a salt thereof or any combination thereof, if a percentage of T cells in a cancer sample or a tumor sample of a subject may be greater than 10% and a percentage of B cells may be less than 5%, or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a percentage of T cells in a cancer or a tumor sample of the subject may be less than 10% and a percentage of B cells, a percentage of plasma cells, or a combination of the percentage of B cells and the percentage of plasma cells may be greater than 10% in the cancer sample of the subject. In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following may be observed when compared to a count prior to treatment: a percentage of T cells, a percentage of B cells, and a percentage of plasma cells. In some embodiments, the cancer or the tumor may be prostate cancer, testicular cancer, breast cancer, brain cancer, pancreatic cancer, colon cancer, thyroid cancer, stomach cancer, lung cancer, ovarian cancer, or any combination thereof.

In an aspect, the present disclosure provides a method of assessing a likelihood of a subject having a cancer or a tumor exhibiting a clinically beneficial response to treatment. The method may comprise: assessing a count of T cells, B cells, and plasma cells in a cancer or a tumor sample of a subject; and calculating, using a computer system, a probability of treatment responsiveness based on (1) a ratio between the count of T cells and a reference count of T cells, (2) a ratio between the count of B cells and a reference count of B cells, and/or (3) a ratio between the count of plasma cells and a reference count of plasma cells. The method may further comprise designating the subject as having a high probability of exhibiting a clinically beneficial response to treatment if the ratio between the count of B cells and the reference count of B cells may be less than 1. The method may further comprise designating the subject as having a high probability of exhibiting a clinically beneficial response to treatment if the ratio between the count of plasma cells and the reference count of plasma cells may be less than 1. In some embodiments, wherein after the administration of the anti-programmed cell death protein 1 agent, the anti-programmed death ligand 1 agent, the anti-programmed death ligand 2 agent, or a salt thereof or any combination thereof, a decrease in at least one of the following may be observed when compared to a count prior to treatment: a count of T cells, a count of B cells, and a count of plasma cells. The cancer or the tumor may be prostate cancer, testicular cancer, breast cancer, brain cancer, pancreatic cancer, colon cancer, thyroid cancer, stomach cancer, lung cancer, ovarian cancer, or any combination thereof. The method may further comprise surgery, radiation therapy, or administering a pharmaceutical agent, wherein the pharmaceutical agent may be an immunotherapeutic agent, a chemotherapeutic agent, a targeted therapeutic agent, a hormonal therapy agent, cell-based therapy agent, radiation therapy agent, or any salt thereof, or any combination thereof.

In an aspect, the present disclosure provides a method of treating a cancer or a tumor in a subject. The method may comprise: administering an anti-programmed cell death protein 1 agent, an anti-programmed death ligand 1 agent, an anti-programmed death ligand 2 agent, or a salt thereof or any combination thereof, if a cancer or a tumor sample of a subject contains at least one cell contains a cluster of differentiation 8 (CD8) protein and no cell contains a cluster of differentiation 19 (CD19) protein or a cluster of differentiation 138 (CD138) protein; or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a cancer or a tumor sample of a subject contains at least one cell contains a CD8 protein and at least one cell contains a CD19 or CD138 protein. In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following may be observed when compared to a count prior to treatment: a count of T cells, a count of B cells, and a count of plasma cells. In some embodiments, after the administration of the anti-programmed cell death protein 1 agent, the anti-programmed death ligand 1 agent, the anti-programmed death ligand 2 agent, or a salt thereof or any combination thereof, and an immunotherapeutic agent or a salt thereof, a decrease in at least one of the following may be observed when compared to a count prior to treatment: a count of T cells, a count of B cells, and a count of plasma cells. In some embodiments, a decrease in a count of T cells, a decrease in a count of B cells, a decrease in a count of plasma cells, or any combination thereof, may be observed. In some embodiments, the method may be a method of treating a cancer or a tumor in the subject. The cancer or the tumor may be prostate cancer, testicular cancer, breast cancer, brain cancer, pancreatic cancer, colon cancer, thyroid cancer, stomach cancer, lung cancer, ovarian cancer, or any combination thereof. In an aspect, the present disclosure provides a method of treating a cancer or a tumor in a subject. The method may comprise administering a therapeutically effective amount of a pharmaceutical agent and an immunotherapeutic agent. In some embodiments, a presence of CD8 protein, the CD19 protein, the CD20 protein, the CD139 protein, the B-cell activating factor protein, the B-cell activating factor-R protein, and the PAX5 protein may be determined by staining, imaging after staining, microscopy, or any combination thereof. The staining may comprise binding of an antibody to the protein. The staining may further comprise binding of a second antibody to a first antibody, wherein the second antibody may contain a fluorescence marker. The method may be repeated after administration of the therapeutic. The cancer sample or the tumor sample may comprise at least one cell comprising a CD8 protein and at least one cell comprising a CD19 protein, wherein the CD8 protein and the CD19 protein have a distance apart of at most 100 microns. The cancer sample or the tumor sample may comprise at least one cell comprising a CD8 protein and at least one cell comprising a CD19 protein, wherein the CD8 protein-containing cell and the CD19 protein-containing cell may have a distance apart of at most about 100 microns.

In an aspect, the present disclosure provides a method of treating a solid cancer in a subject in need thereof. The method may comprise: administering a therapeutically effective amount of at least one B-cell activating factor (BAFF) inhibitor and/or at least one B-cell activating factor receptor (BAFF-R) inhibitor, or a salt thereof; and administering a therapeutically effective amount of at least one checkpoint inhibitor or a salt thereof. The subject may be not an appropriate candidate for a monotherapy comprising the at least one checkpoint inhibitor. The solid cancer may be at least partially resistant to a monotherapy comprising the at least one checkpoint inhibitor. The solid cancer may have an incomplete response to a monotherapy comprising the at least one checkpoint inhibitor. The subject may be currently receiving treatment of a monotherapy comprising the at least one checkpoint inhibitor. In some embodiments, the BAFF inhibitor may be tabalumab or a salt thereof. The BAFF inhibitor may be blisibimod or a salt thereof. The BAFF inhibitor may be belimumab or a salt thereof. The BAFF inhibitor may be atacicept or a salt thereof. The BAFF inhibitor may be an anti-BAFF-R antibody or a portion thereof. The anti-BAFF-R antibody may be an anti-BAFF-R3 antibody. The at least one checkpoint inhibitor or a salt thereof may be selected from the group consisting of a monoclonal antibody, a humanized antibody, a chimeric antibody, a fully human antibody, a fusion protein, a salt of any one thereof, and any combination thereof. The checkpoint inhibitor may be a biologic or a small molecule. A checkpoint protein may be selected from the group consisting of PDl, PDL1, CTLA-4, PDL2, TIM3, LAG3, VISTA, B7.1, B7-H3, B7-H4, BTLA, HVEM, GAL9, KIR, 2B4, CD137, CD27, CD28, CD40, CD122, CD160, CGEN-15049, CHK 1, CCR4, CHK2, Ox40, GITR, ICOS, IDO, A2aR, B-7 family ligands, or any combination thereof. The checkpoint inhibitor may inhibit a checkpoint protein selected from the group consisting of PDl, PDL1, CTLA-4, PDL2, TIM3, LAG3, VISTA, B7.1, B7-H3, B7-H4, BTLA, HVEM, GAL9, KIR, 2B4, CD137, CD27, CD28, CD40, CD122, CD160, CGEN-15049, CHK 1, CCR4, CHK2, GITR, ICOS, IDO, Ox40, A2aR, B-7 family ligands, and any combination thereof. The checkpoint inhibitor may interact with a ligand of a checkpoint protein comprising PDl, PDL1, CTLA-4, PDL2, TIM3, LAG3, VISTA, B7.1, B7-H3, B7-H4, BTLA, HVEM, GAL9, KIR, 2B4, CD137, CD27, CD28, CD40, CD122, CD160, CGEN-15049, CHK 1, CCR4, CHK2, GITR, ICOS, IDO, Ox40, A2aR, B-7 family ligands, or a combination thereof. The checkpoint inhibitor may interact with a ligand of a checkpoint protein selected from the group consisting of PDl, PDL1, CTLA-4, PDL2, TIM3, LAG3, VISTA, B7.1, B7-H3, B7-H4, BTLA, HVEM, GAL9, KIR, 2B4, CD137, CD27, CD28, CD40, CD160, CGEN-15049, CHK 1, CCR4, CHK2, GITR, ICOS, Ox40, A2aR, B-7 family ligands, and a combination thereof. In some embodiments, the solid cancer may be selected from the group consisting of urogenital, gynecological, lung, gastrointestinal, head and neck cancer, malignant glioblastoma, malignant mesothelioma, non-metastatic or metastatic breast cancer, malignant melanoma, Merkel Cell Carcinoma or bone and soft tissue sarcoma, non-small cell lung cancer (NSCLC), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, esophageal cancer, hepatocellular cancer, cholangiocellular cancer, small cell lung cancer, and combinations thereof. The method may further comprise administering an immunotherapeutic agent, a chemotherapeutic agent, or a targeted therapy to the subject prior to, simultaneously with, or after the treating. The treating may be evaluated by a decrease in tumor growth, induction of tumor cell death, tumor regression, preventing or delaying tumor recurrence, preventing or delaying tumor spread, or any combination thereof.

In an aspect, the current disclosure may provide a method for treating a cancer in a subject. The method may comprise: screening a cancer sample or a tumor sample isolated from the subject, wherein the screening may comprise determining a presence or an absence of a genomic polymorphism or genotype that may be correlative to a treatment outcome of the cancer; administering a therapeutically effective amount of at least one B-cell activating factor (BAFF) inhibitor, or a salt thereof, and/or at least one B-cell activating factor receptor (BAFF-R) inhibitor, or a salt thereof; and administering a therapeutically effective amount of at least one PD-L1 inhibitor or a salt thereof, wherein the administering of the at least one B-cell activating factor (BAFF) inhibitor, or salt thereof, and/or the at least one B-cell activating factor receptor (BAFF-R), or salt thereof, and the PD-L1 inhibitor, or salt thereof may be based on the screening for the presence or absence of the genomic polymorphism or genotype that may be correlative to the treatment outcome of the cancer. In some embodiments, the PD-L1 inhibitor or a salt thereof may comprise nivolumab, pembrolizumab, pidilizumab, BGB-A31, MEDI680 (AMP-514), a salt of any one thereof, or any combination thereof. In some embodiments, the PD-L1 inhibitor or a salt thereof may be selected from the group consisting of nivolumab, pembrolizumab, pidilizumab, BGB-A31, MEDI0680 (AMP-514), a salt of any one thereof, and any combination thereof. The method may further comprise, before the administering: determining a number of an aggregate in the cancer sample or the tumor sample isolated from the subject, wherein the aggregate may comprise at least a first cell and a second cell, wherein the first cell and the second cell may be of a different cell type, and wherein a population of the first cells and a population of the second cells may be in contact with each other. In some embodiments, the determining of the number of the aggregate may be determined by staining, microscopy, imaging after staining, or any combination thereof. The aggregate may comprise at least about 5 cells. The aggregate may comprise from about 5 to about 100,000 cells. The cancer sample or the tumor sample may comprise at least 1 aggregate. The cancer sample or the tumor sample may comprise about 1 aggregate to about 20 aggregates. The aggregate may be an immune cell aggregate. The cell type of the first cell may be a lymphoid cell, a plasma cell, a myeloid cell, or a mast cell. The cell type of the second cell may be a lymphoid cell, a plasma cell, a myeloid cell, or a mast cell. The aggregate may be a lymphoid aggregate. In some embodiments, the aggregate may comprise a germinal center. The aggregate may not comprise a germinal center. The aggregate may be located at an invasive margin, a perivascular space, a tumor parenchyma of the cancer, or any combination thereof. A first cell may be a B-cell, and wherein the aggregate may comprise a B-cell density of at least about 1 cell/mm² by area of the aggregate. A first cell may be a B-cell, and wherein the aggregate may comprise a B-cell density of from about 1 cell/mm² by area of the aggregate to about 1000 cells/mm² by area of the aggregate. In some embodiments, a first cell may be a B-cell, and wherein the aggregate may comprise a B-cell density of at least about 100 cells/mm² by area of the aggregate. A first cell may be a B-cell, and wherein the aggregate may comprise at least about 1 B-cell. A first cell may be a B-cell, and wherein the aggregate may comprise at least about 10 B-cells. A second cell may be a T-cell. A second cell may be a T-cell, and wherein the aggregate may comprise at least about 10 T-cells. A second cell may be a T-cell, and wherein the aggregate may comprise about 100 T-cells. The aggregate may comprise BAFF and BAFF-R. The aggregate may comprise BAFF and at least one of: CD19, CD20, or CD138. The first cell or second cell in the aggregate may express a protein. The protein may be BAFF, BAFF-R, CD3, CD19, CD20, PAX5, or PD-L1. The determining of the number of the aggregate may be assessed by hematoxylin staining, eosin staining, DAPI staining, or any combination thereof.

In an aspect, the current disclosure provides a method of detecting a number aggregates in a tissue sample of a subject. The method may comprise staining the tissue sample that may have been previously diagnosed with a cancer with hematoxylin, eosin, or DAPI, wherein the staining may detect CD3, CD19, CD20, PAX5, BAFF-R, BAFF, PD-L1, or any combination thereof. In an aspect, the current disclosure provides a method comprising determining a presence of at least one aggregate of T-cells in a cancer sample or a tumor sample of a subject, wherein the at least one aggregate T-cells may be in proximity to at least one B-cell, wherein the B-cell may express at least one protein selected from the group consisting of: cluster of differentiation 19 (CD19) protein, cluster of differentiation 20 protein (CD20), cluster of differentiation 138 (CD138) protein, and any combination thereof. The determining may comprise staining, microscopy, imaging after staining, or any combination thereof. The aggregate may comprise at least about 5 cells. The aggregate may comprise about 5 cells to about 100,000 cells. In some embodiments, the cancer sample or the tumor sample may comprise at least 1 aggregate. The aggregate may be an immune cell aggregate. The aggregate may be a lymphoid aggregate. The aggregate may comprise a germinal center. The aggregate may not comprise a germinal center. The aggregate may be located at an invasive margin, a perivascular space, or a tumor parenchyma of the cancer, or any combination thereof. The method may be a method of determining if a cancer or a tumor may be responsive to a single therapeutic agent. The single therapeutic agent may be an anti-programmed cell death protein 1 agent, an anti-programmed death ligand 1 agent, an anti-programmed death ligand 2 agent, or a salt thereof or any combination thereof. In some embodiments, the at least one aggregate of T-cells may be in proximity to at least one B-cell, wherein the B-cell may express at least one protein selected from the group consisting of: cluster of differentiation 19 (CD19) protein, cluster of differentiation 20 protein (CD20), cluster of differentiation 138 (CD138) protein, and any combination thereof. The method may further comprise selecting a therapeutic regimen comprising: an anti-programmed cell death protein 1 agent, an anti-programmed death ligand 1 agent, an anti-programmed death ligand 2 agent, or a salt thereof or any combination thereof, and an therapeutic agent or a salt thereof, wherein the at least one aggregate of T-cells may be in proximity to at least one B-cell, wherein the B-cell may express at least one protein selected from the group consisting of: cluster of differentiation 19 (CD19) protein, cluster of differentiation 20 protein (CD20), cluster of differentiation 138 (CD138) protein, and any combination thereof. In some embodiments, the anti-programmed cell death protein 1 agent or a salt thereof may comprise docetaxel, nivolumab, pembrolizumab, a salt of any of these, or any combination thereof. The anti-programmed cell death protein 1 agent or a salt thereof may be selected from the group consisting of docetaxel, nivolumab, pembrolizumab, a salt of any of these, and any combination thereof. The therapeutic agent or a salt thereof may comprise ardenermin, briobacept, blisibimod, belimumab, a transmembrane activator and calcium-modulator and cyclophilin ligand interactorcept, tabalumab, atacicept, RCT-18, a salt of any of these, or any combination thereof. The therapeutic agent or a salt thereof may be selected from the group consisting of ardenermin, briobacept, blisibimod, belimumab, a transmembrane activator and calcium-modulator and cyclophilin ligand interactorcept, tabalumab, atacicept, RCT-18, a salt of any of these, and any combination thereof Δt least one of the following proteins may be present in the at least one B-cell, the at least one aggregate of T-cells, or both: CD3, CD19, CD20, PAX5, BAFF-R, BAFF, and PD-L1. The method may be a method of diagnosing a cancer or a tumor in the subject. The method may be a method of determining if the cancer or the tumor may be becoming non-responsive to a single agent therapy.

In an aspect, the current disclosure provides a method comprising obtaining a cancer sample or a tumor sample of a subject and identifying a presence of a T-cell interacting with at least one cell bearing at least one protein selected from the group consisting of: cluster of differentiation 19 (CD19) protein, cluster of differentiation 20 protein (CD20), cluster of differentiation 138 (CD138) protein, and any combination thereof. The at least one cell may be a B-cell. Staining the B-cell may detect CD3, CD19, CD20, PAX5, BAFF-R, BAFF, or PD-L1, or any combination thereof. In an aspect, the current disclosure provides a method of treating a cancer in a subject. The method may comprise: administering a therapeutically effective amount of an immunotherapeutic agent or a salt thereof and a therapeutically effective amount of a checkpoint inhibitor. The cancer or the tumor may comprise a T-cell interacting with at least one cell bearing at least one protein selected from the group consisting of: cluster of differentiation 19 (CD19) protein, cluster of differentiation 20 protein (CD20), cluster of differentiation 138 (CD138) protein, and any combination thereof. The at least one cell may be a B-cell. Staining the B-cell may detect CD3, CD19, CD20, PAX5, BAFF-R, BAFF, PD-L1, or any combination thereof. In some aspects, the current disclosure provides a method. The method may comprise: obtaining a cancer sample or a tumor sample from a subject; identifying a presence of B-cell activating factor (BAFF) and B-cell activating factor receptor (BAFF-R) in the cancer sample or the tumor sample; or identifying a presence of B-cell activating factor (BAFF) and at least one protein selected from the group consisting of: cluster of differentiation 19 (CD19) protein, cluster of differentiation 20 protein (CD20), cluster of differentiation 138 (CD138) protein, and any combination thereof in the cancer sample or the tumor sample. The method may further comprise administering a therapeutically effective amount of an anti-programmed cell death protein 1 agent, an anti-programmed death ligand 1 agent, an anti-programmed death ligand 2 agent, or a salt thereof or any combination thereof, and an immunotherapeutic agent or a salt thereof. In some embodiments, the cancer or the tumor may comprise a T-cell interacting with at least one cell bearing at least one protein selected from the group consisting of: cluster of differentiation 19 (CD19) protein, cluster of differentiation 20 protein (CD20), cluster of differentiation 138 (CD138) protein, and any combination thereof. The cancer sample or the tumor sample may comprise at least one aggregate of T-cells in proximity to at least one B-cell, wherein the B-cell expresses at least one protein may be selected from the group consisting of: cluster of differentiation 19 (CD19) protein, cluster of differentiation 20 protein (CD20), cluster of differentiation 138 (CD138) protein, and any combination thereof. In some aspects, the current disclosure may provide a method of determining a first amount of cluster of differentiation 8 protein in a cancer sample or a tumor sample of a subject prior to a single agent treatment and determining a second amount of cluster of differentiation 8 protein in the cancer sample or the tumor sample of the subject on or after day 20 of the single agent treatment.

In some aspects, the current disclosure provides a method of selecting combination therapeutic regimen. The method may comprise: selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof wherein the second amount of cluster of differentiation 8 protein in the cancer sample or the tumor sample of the subject on or after day 20 of the single agent treatment may be higher than the first amount of cluster of differentiation 8 protein in the cancer sample or the tumor sample of the subject prior to the single agent treatment. One embodiment provides a method comprising: (I) determining expression levels of: (a) a gene listed in Table 4, a protein product thereof, a variant thereof, or a fragment thereof, and (b) a gene that codes for the BAFF protein or the BAFF-receptor protein, a variant thereof, or a fragment thereof, wherein the expression levels are determined in a biological sample isolated from a tumor microenvironment within the subject; and (II) identifying the subject as: (i) responsive to a combination therapy comprising an immunotherapy and agent that is capable of regulating activity of a gene that codes for a BAFF protein or a BAFF-receptor protein; or (ii) resistant to an immunotherapy comprising a single agent, wherein the identifying is based on expression levels of (a) and (b). One embodiment provides a method of identifying a subject as responsive to a combination therapy comprising an immunotherapy and an agent capable of regulating activity of a BAFF protein or a BAFF-receptor protein, comprising: (I) determining expression levels of (a) a gene listed in Table 4, a protein product thereof, a variant thereof, or a fragment thereof; and an expression level of (b) a gene that codes for the BAFF protein or the BAFF-receptor protein, a variant thereof, or a fragment thereof, wherein the expression levels are determined in a biological sample isolated from a tumor microenvironment within the subject; and (II) identifying the subject as responsive to the combination therapy based on the expression levels of (a) and (b). One embodiment provides a method of identifying a subject as resistant to an immunotherapy comprising a single agent, comprising: (I) determining expression levels of (a) a gene listed in Table 4, a protein product thereof, a variant thereof, or a fragment thereof; and (b) a gene that codes for the BAFF protein or the BAFF-receptor protein, a variant thereof, or a fragment thereof, wherein the expression levels are determined in a biological sample isolated from a tumor microenvironment within the subject; and (II) identifying the subject as resistant to the immunotherapy comprising a single agent based on the expression levels of (a) and (b). In some embodiments, the method further comprises determining an expression level of (c) a gene listed in Table 5, a protein product thereof, a variant thereof, or a fragment thereof. In some embodiments, the expression levels of (a), (b), and (c) are independently determined by RNA-seq. In some embodiments, the expression levels of (a), (b), and (c) are independently determined by immunohistochemistry or immunofluorescence or multiplexed ion beam based imaging methods. In some embodiments, the method further comprises determining a correlation between the expression levels of (a) and (b). In some embodiments, the method further comprises determining a correlation between the expression levels of (b) and (c). In some embodiments, the method further comprises determining a correlation between the expression levels of (a) and (c). In some embodiments, the correlations between expression levels of (a) and (b), (a) and (c), and (b) and (c), are independently determined by a t-test, a paired t-test, an analysis of variance (ANOVA), a repeated measures ANOVA, a simple linear regression, a nonlinear regression, a multiple linear regression, a multiple nonlinear regression, a Wilcoxon signed-rank test, a MannWhitney test, a Kruskal-Wallis test, a Friedman test, a Spearman rank order correlation coefficient, a Kendall Tau analysis, or a nonparametric regression test. In some embodiments, the correlations between expression levels of (a) and (b), and (b) and (c), are independently determined by the Spearman rank order correlation coefficient. In some embodiments, the correlation between expression levels of (a) and (b) comprises a Spearman's rank order correlation coefficient that is higher than a Spearman's rank order correlation coefficient for the correlation between expression levels of (b) and (c). In some embodiments, genes listed in Table 4 are representatives of a T-cell inflamed phenotype of the tumor microenvironment. In some embodiments, the immunotherapy comprises administering an immune checkpoint protein inhibitor. In some embodiments, the immune checkpoint protein comprises PD-1, PD-L1, PD-L2, CTLA-4, A2AR, B7-H3, B7-H4, BTLA, IDO, KIR, LAG3, TIM-3, VISTA, CD160, TIGIT or PSGL-1. In some embodiments, the immune checkpoint protein comprises PD-1 or PD-L1. In some embodiments, the immune checkpoint protein inhibitor comprises at least one of a PD-1 inhibitor, a PD-L1 inhibitor, a PD-L2 inhibitor, or a salt thereof, or any combination thereof. One embodiment provides a method of identifying a subject for initiating or continuing a combination therapy or an immunotherapy comprising a single agent, comprising: (I) determining expression levels of: (a) a gene listed in Table 4, a protein product thereof, a variant thereof, or a fragment thereof, and (b) a gene that codes for a BAFF protein or a BAFF-receptor protein, a variant thereof, or a fragment thereof, wherein the expression levels are determined in a biological sample isolated from a tumor microenvironment within a subject; and (II) identifying the subject as (i) a suitable candidate for the initiating or continuing the combination therapy; or, (ii) a suitable candidate for the initiating or continuing the immunotherapy comprising the single agent, wherein the identifying is based on the expression levels of (a) and (b). In some embodiments, the combination therapy comprises an immunotherapy and an agent capable of regulating activity of a gene that codes for a BAFF protein or a BAFF-receptor protein. In some embodiments, the combination therapy comprises an immunotherapy comprising administering a single agent. In some embodiments, the subject has previously been identified as resistant to the immunotherapy comprising the single agent. In some embodiments, the subject has previously been administered the immunotherapy comprising the single agent. In some embodiments, the subject has previously been administered the combination therapy. In some embodiments, the method further comprises determining an expression level of (c) a gene listed in Table 5, a protein product thereof, a variant thereof, or a fragment thereof. In some embodiments, the expression levels of (a), (b), and (c), are independently determined by RNA-seq. In some embodiments, the method further comprises determining a correlation between the expression levels of (a) and (b). In some embodiments, the method further comprises determining a correlation between the expression levels of (b) and (c). In some embodiments, the method further comprises determining a correlation between the expression levels of (a) and (c). In some embodiments, the correlations between (a) and (b), (a) and (c), and (b) and (c), are independently determined by a t-test, a paired t-test, an analysis of variance (ANOVA), a repeated measures ANOVA, a simple linear regression, a nonlinear regression, a multiple linear regression, a multiple nonlinear regression, a Wilcoxon signed-rank test, a MannWhitney test, a Kruskal-Wallis test, a Friedman test, a Spearman rank order correlation coefficient, a Kendall Tau analysis, or a nonparametric regression test. In some embodiments, the correlations between (a) and (b), (a) and (c), and (b) and (c), are independently determined by the Spearman rank order correlation coefficient. In some embodiments, the correlation between expression levels of (a) and (b) comprises a Spearman's rank order correlation coefficient that is higher than a Spearman's rank order correlation coefficient for the correlation between expression levels of (b) and (c). In some embodiments, genes listed in Table 4 are representatives of a T-cell inflamed phenotype of the tumor microenvironment. In some embodiments, the single agent comprises administering an immune checkpoint protein inhibitor. In some embodiments, the immunotherapy comprises administering an immune checkpoint protein inhibitor. In some embodiments, the immune checkpoint protein comprises PD-1, PD-L1, PD-L2, CTLA-4, A2AR, B7-H3, B7-H4, BTLA, IDO, KIR, LAG3, TIM-3, VISTA, CD160, TIGIT or PSGL-1. In some embodiments, the immune checkpoint protein comprises the PD-1 or the PD-L1. In some embodiments, the immune checkpoint protein inhibitor comprises at least one of a PD-1 inhibitor, a PD-L1 inhibitor, a PD-L2 inhibitor, or a salt thereof, or any combination thereof.

In some embodiments provided herein are methods of treating a cancer in a subject resistant to an immunotherapy comprising a single agent, comprising: administering a combination of (i) a therapeutically effective amount of an agent that regulates activity of a BAFF protein or a BAFF-receptor protein, and (ii) the immunotherapy comprising the single agent.

In some embodiments, the subject has previously been identified as resistant to the immunotherapy comprising the single agent by a method comprising: (I) determining expression levels of (a) a gene listed in Table 4, a protein product thereof, a variant thereof, or a fragment thereof, and (b) a gene that codes for a BAFF protein or a BAFF-receptor protein, a variant thereof, or a fragment thereof, wherein the expression levels are determined in a biological sample isolated from a tumor microenvironment within the subject; and (II) identifying the subject as resistant to the combination therapy comprising an immunotherapy and agent that regulates activity of a BAFF protein or a BAFF-receptor protein, wherein the identifying is based on the expression levels of (a) and (b). In some embodiments, the method further comprises determining and expression level of (c) a gene listed in Table 5, a protein product thereof, a variant thereof, or a fragment thereof. In some embodiments, the expression levels of (a), (b), and (c) are independently determined by RNA-seq. In some embodiments, the method further comprises determining a correlation between the expression levels of (a) and (b). In some embodiments, the method further comprises determining a correlation between the expression levels of (b) and (c). In some embodiments, the method further comprises determining a correlation between the expression levels of (a) and (c). In some embodiments, the correlations between expression levels of (a) and (b), (a) and (c), and (b) and (c), are independently determined by a t-test, a paired t-test, an analysis of variance (ANOVA), a repeated measures ANOVA, a simple linear regression, a nonlinear regression, a multiple linear regression, a multiple nonlinear regression, a Wilcoxon signed-rank test, a MannWhitney test, a Kruskal-Wallis test, a Friedman test, a Spearman rank order correlation coefficient, a Kendall Tau analysis, or a nonparametric regression test. In some embodiments, the correlations between expression levels of (a) and (b), (a) and (c), and (b) and (c), are independently determined by the Spearman rank order correlation coefficient. In some embodiments, the correlation between expression levels of (a) and (b) comprises a Spearman's rank order correlation coefficient that is higher than a Spearman's rank order correlation coefficient for the correlation between expression levels of (b) and (c). In some embodiments, genes listed in Table 4 are representatives of a T-cell inflamed phenotype of the tumor microenvironment. In some embodiments, the immunotherapy comprising the single agent comprises administering an immune checkpoint protein inhibitor. In some embodiments, the immune checkpoint protein comprises PD-1, PD-L1, PD-L2, CTLA-4, A2AR, B7-H3, B7-H4, BTLA, IDO, KIR, LAG3, TIM-3, VISTA, CD160, TIGIT or PSGL-1. In some embodiments, the immune checkpoint protein comprises PD-1 or PD-L1. In some embodiments, the immune checkpoint protein inhibitor comprises at least one of a PD-1 inhibitor, a PD-L1 inhibitor, a PD-L2 inhibitor, or a salt thereof, or any combination thereof.

In some embodiments provided herein are methods comprising: (I) determining expression levels of (a) a gene listed in Table 4, a protein product thereof, a variant thereof, or a fragment thereof; and (b) a gene that codes for a BAFF protein or a BAFF-receptor protein, a variant thereof, or a fragment thereof, wherein the expression levels are determined in a biological sample isolated from a tumor microenvironment within a subject; and (II) administering a therapeutically effective amount of an agent that regulates activity of a BAFF protein or a BAFF-receptor protein, wherein the administering is based on the expression levels of (a) and (b) in the biological sample. In some embodiments, the method further comprises determining an expression level of (c) a gene listed in Table 5, a protein product thereof, a variant thereof, or a fragment thereof. In some embodiments, the expression levels of (a), (b), and (c) are independently determined by RNA-seq. In some embodiments, the method further comprises determining a correlation between the expression levels of (a) and (b). In some embodiments, the method further comprises determining a correlation between the expression levels of (b) and (c). In some embodiments, the method further comprises determining a correlation between the expression levels of (a) and (c). In some embodiments, the correlations between expression levels of (a) and (b), (a) and (c), and (b) and (c), are independently determined by a t-test, a paired t-test, an analysis of variance (ANOVA), a repeated measures ANOVA, a simple linear regression, a nonlinear regression, a multiple linear regression, a multiple nonlinear regression, a Wilcoxon signed-rank test, a MannWhitney test, a Kruskal-Wallis test, a Friedman test, a Spearman rank order correlation coefficient, a Kendall Tau analysis, or a nonparametric regression test. In some embodiments, the correlations between expression levels of (a) and (b), (a) and (c), and (b) and (c), are independently determined by the Spearman rank order correlation coefficient. In some embodiments, the correlation between expression levels of (a) and (b) comprises a Spearman's rank order correlation coefficient that is higher than a Spearman's rank order correlation coefficient for the correlation between expression levels of (b) and (c). In some embodiments, genes listed in Table 4 are representatives of a T-cell inflamed phenotype of the tumor microenvironment. In some embodiments, the method further comprises administering an immunotherapy. In some embodiments, the immunotherapy comprises administering an immune checkpoint protein inhibitor. In some embodiments, the immune checkpoint protein comprises PD-1, PD-L1, PD-L2, CTLA-4, A2AR, B7-H3, B7-H4, BTLA, IDO, KIR, LAG3, TIM-3, VISTA, CD160, TIGIT or PSGL-1. In some embodiments, the immune checkpoint protein comprises PD-1 or PD-L1. In some embodiments, the immune checkpoint protein inhibitor comprises at least one of a PD-1 inhibitor, a PD-L1 inhibitor, a PD-L2 inhibitor, or a salt thereof, or any combination thereof. In some embodiments, the agent that regulates activity of the BAFF protein or the BAFF-receptor protein comprises a B-cell activating factor (BAFF) inhibitor, or a salt thereof, a B-cell activating factor receptor (BAFF-R) inhibitor, or a salt thereof, or any combination thereof. In some embodiments, the combination therapy comprises administering a therapeutically effective amount of a B-cell activating factor (BAFF) inhibitor, or a salt thereof, a B-cell activating factor receptor (BAFF-R) inhibitor, or a salt thereof, or any combination thereof and at least one of a PD-1 inhibitor, a PD-L1 inhibitor, a PD-L2 inhibitor, or a salt thereof, or any combination thereof. In some embodiments, the administering of the B-cell activating factor (BAFF) inhibitor, or a salt thereof, a B-cell activating factor receptor (BAFF-R) inhibitor, or a salt thereof, or any combination thereof the and the administering the at least one of a PD-1 inhibitor, a PD-L1 inhibitor, a PD-L2 inhibitor, or a salt thereof, or any combination thereof, is concurrent or sequential. In some embodiments, the administering of the B-cell activating factor (BAFF) inhibitor, or a salt thereof, a B-cell activating factor receptor (BAFF-R) inhibitor, or a salt thereof, or any combination thereof precedes the administering the at least one of a PD-1 inhibitor, a PD-L1 inhibitor, a PD-L2 inhibitor, or a salt thereof, or any combination thereof. In some embodiments, the administering of the B-cell activating factor (BAFF) inhibitor, or a salt thereof, a B-cell activating factor receptor (BAFF-R) inhibitor, or a salt thereof, or any combination thereof follows the administering the at least one of a PD-1 inhibitor, a PD-L1 inhibitor, a PD-L2 inhibitor, or a salt thereof, or any combination thereof. In some embodiments, the method further comprises conveying result of expression levels of one or more of (a), (b), and (c) through a computer communication medium.

In some embodiments provided herein are methods of identifying an inflammation status of a tumor comprising: determining expression levels of: (a) a gene listed in Table 4, a protein product thereof, a variant thereof, or a fragment thereof; and (b) a gene that codes for a BAFF protein or a BAFF-receptor protein, a variant thereof, or a fragment thereof, wherein the expression levels are determined in a biological sample isolated from a tumor microenvironment within a subject. In some embodiments, the inflammation status comprises a T-cell inflamed signature. In some embodiments, the method further comprises administering a B-cell activating factor (BAFF) inhibitor, or a salt thereof, a B-cell activating factor receptor (BAFF-R) inhibitor, or a salt thereof, or any combination thereof the and at least one of a PD-1 inhibitor, a PD-L1 inhibitor, a PD-L2 inhibitor, or a salt thereof, based on the T-cell inflamed signature. In some embodiments provided herein are methods comprising: determining expression levels of: a gene that codes for the BAFF protein or the BAFF-receptor protein, a variant thereof, or a fragment thereof, wherein the expression levels are determined in a biological sample isolated from a tumor microenvironment within the subject; and identifying the subject as: responsive to a combination therapy comprising an immunotherapy and agent that is capable of regulating activity of a gene that codes for a BAFF protein or a BAFF-receptor protein; or resistant to an immunotherapy comprising a single agent, wherein the identifying is based on expression level of the gene. In some embodiments, the method further comprises administering a B-cell activating factor (BAFF) inhibitor, or a salt thereof, a B-cell activating factor receptor (BAFF-R) inhibitor, or a salt thereof, or any combination thereof the and at least one of a PD-1 inhibitor, a PD-L1 inhibitor, a PD-L2 inhibitor, or a salt thereof, based on the T-cell inflamed tumor signature.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, patent applications, and NCBI accession numbers mentioned in this specification are herein incorporated by reference to the same extent as if each individual publication, patent, or patent application was specifically and individually indicated to be incorporated by reference, and as if set forth in their entireties. In the event of a conflict between a term as used herein and the term as defined in the incorporated reference, the definition of this disclosure controls.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “figure” and “FIG.” herein), of which:

FIG. 1 provides a representative example of a matrix that defines cellular boundaries.

FIG. 2 provides a 9×9 representative example, which corresponds with FIG. 1 and is an example of an intensity matrix, wherein the relative intensity of each marker of interest per pixel is defined.

FIG. 3 provides an example of pixel labeling.

FIG. 4 provides frequency polygons of pixel staining intensities.

FIG. 5 shows an example of positive intensity thresholding with heatmap visualization.

FIG. 6 provides an example of an adjacency graph.

FIG. 7 parts A-C provides an example heatmap.

FIG. 8 shows an adjacency heatmap of a subject that did not respond to treatment, or a non-responder subject.

FIG. 9 shows a fraction of adjacency heatmap.

FIG. 10 shows an adjacency heatmap of the difference between the heatmap of a responder minus the heatmap of a non-responder.

FIG. 11 illustrates the use of pixel-based analytics for diagnostic and therapeutic development.

FIG. 12 shows a computer control system.

FIG. 13A-13C and FIG. 14A-14C illustrate three types of tumors before and after treatment, including Type II resistant tumors.

FIG. 15A-15B illustrates the proliferation of CD8⁺ T cells and cancer cells in Type II resistant tumors.

FIG. 16 illustrates the proliferation of CD8⁺ T cells and cancer cells Type I and Type II resistant tumors.

FIG. 17 illustrates the Type II resistant tumors are associated with high PD-L1 expression at baseline.

FIG. 18 illustrates BAFF expression in samples obtained before anti-PD1 therapy according to clinical response.

FIG. 19 illustrates BAFF-R expression in samples obtained before anti-PD1 therapy according to clinical response.

FIG. 20 illustrates BAFF/BAFF-R expression in breast cancer, non-small cell lung cancer (squamous), non-small cell lung cancer (adeno), and head and neck squamous cell carcinoma.

FIG. 21 illustrates a correlation heatmap demonstrating the T-cell inflamed signature of Acute Myeloid Leukemia with BAFF.

FIG. 22 illustrates a correlation heatmap demonstrating the T-cell inflamed signature of Adrenocortical Carcinoma with BAFF.

FIG. 23 illustrates a correlation heatmap demonstrating the T-cell inflamed signature of Head and Neck Squamous Cell Carcinoma with BAFF.

FIG. 24 illustrates a correlation heatmap demonstrating the T-cell inflamed signature of Breast Invasive Carcinoma with BAFF.

FIG. 25 illustrates a correlation heatmap demonstrating the T-cell inflamed signature of Lung Adenocarcinoma with BAFF.

FIG. 26 illustrates a correlation heatmap demonstrating the T-cell inflamed signature of Lung Squamous Cell Carcinoma with BAFF.

FIG. 27 illustrates a correlation heatmap demonstrating the T-cell inflamed signature of Bladder Urothelial Carcinoma with BAFF.

FIG. 28 illustrates a correlation heatmap demonstrating the T-cell inflamed signature of Skin Cutaneous Melanoma with BAFF.

FIG. 29 illustrates a correlation heatmap demonstrating the T-cell inflamed signature of Mesothelioma with BAFF.

FIG. 30 illustrates a correlation heatmap demonstrating the T-cell inflamed signature of Brain lower grade glioma with BAFF.

FIG. 31 illustrates a correlation heatmap demonstrating the T-cell inflamed signature of Cervical squamous cell carcinoma and Endocervical adenocarcinoma with BAFF.

FIG. 32 illustrates a correlation heatmap demonstrating the T-cell inflamed signature of Cholangiocarcinoma with BAFF.

FIG. 33 illustrates a correlation heatmap demonstrating the T-cell inflamed signature of Colon adenocarcinoma with BAFF.

FIG. 34 illustrates a correlation heatmap demonstrating the T-cell inflamed signature of Esophageal carcinoma with BAFF.

FIG. 35 illustrates a correlation heatmap demonstrating the T-cell inflamed signature of Glioblastoma multiforme with BAFF.

FIG. 36 illustrates a correlation heatmap demonstrating the T-cell inflamed signature of High-risk Wilms Tumor with BAFF.

FIG. 37 illustrates a correlation heatmap demonstrating the T-cell inflamed signature of Kidney chromophobe with BAFF.

FIG. 38 illustrates a correlation heatmap demonstrating the T-cell inflamed signature of Kidney renal clear cell carcinoma with BAFF.

FIG. 39 illustrates a correlation heatmap demonstrating the T-cell inflamed signature of Kidney renal papillary cell carcinoma with BAFF.

FIG. 40 illustrates a correlation heatmap demonstrating the T-cell inflamed signature of Liver hepatocellular carcinoma with BAFF.

FIG. 41 illustrates a correlation heatmap demonstrating the T-cell inflamed signature of Lymphoid neoplasm diffuse large B-cell lymphoma with BAFF.

FIG. 42 illustrates a correlation heatmap demonstrating the T-cell inflamed signature of Stomach adenocarcinoma with BAFF.

FIG. 43 illustrates a correlation heatmap demonstrating the T-cell inflamed signature of neuroblastoma with BAFF.

FIG. 44 illustrates a correlation heatmap demonstrating the T-cell inflamed signature of Ovarian serous cystadenocarcinoma with BAFF.

FIG. 45 illustrates a correlation heatmap demonstrating the T-cell inflamed signature of Pancreatic adenocarcinoma with BAFF.

FIG. 46 illustrates a correlation heatmap demonstrating the T-cell inflamed signature of Pheochromocytoma and Paraganglioma with BAFF.

FIG. 47 illustrates a correlation heatmap demonstrating the T-cell inflamed signature of Prostate adenocarcinoma with BAFF.

FIG. 48 illustrates a correlation heatmap demonstrating the T-cell inflamed signature of Rectum adenocarcinoma with BAFF.

FIG. 49 illustrates a correlation heatmap demonstrating the T-cell inflamed signature of Rhabdoid tumor with BAFF.

FIG. 50 illustrates a correlation heatmap demonstrating the T-cell inflamed signature of Sarcoma with BAFF.

FIG. 51 illustrates a correlation heatmap demonstrating the T-cell inflamed signature of Testicular germ cell tumors with BAFF.

FIG. 52 illustrates a correlation heatmap demonstrating the T-cell inflamed signature of Thymoma with BAFF.

FIG. 53 illustrates a correlation heatmap demonstrating the T-cell inflamed signature of Thyroid carcinoma with BAFF.

FIG. 54 illustrates a correlation heatmap demonstrating the T-cell inflamed signature of Uterine carcinosarcoma with BAFF.

FIG. 55 illustrates a correlation heatmap demonstrating the T-cell inflamed signature of Uterine corpus endothelial carcinoma with BAFF.

FIG. 56 illustrates a correlation heatmap demonstrating the T-cell inflamed signature of uveal melanoma with BAFF.

FIG. 57 illustrates an aggregate heatmap for correlation between BAFF expression and T-cell inflamed signatures.

FIG. 58 illustrates sample data points for BAFF expression (TCGA identifier ENSG00000102524.10) as a boxplot with outliers and ordered left to right based on increasing median FPKM (Fragments Per Kilobase of transcript per Million mapped reads).

### DETAILED DESCRIPTION

As used herein, the singular forms “a”, “an”, and “the” include plural references unless the context clearly dictates otherwise. Any reference to “or” herein may be intended to encompass “and/or” unless otherwise stated.

As used herein, the term “about” may mean the referenced numeric indication plus or minus 15% of that referenced numeric indication.

The term “fragment,” as used herein, may be a portion of a sequence, a subset that may be shorter than a full-length sequence. A fragment may be a portion of a gene. A fragment may be a portion of a peptide or protein. A fragment may be a portion of an amino acid sequence. A fragment may be a portion of an oligonucleotide sequence. A fragment may be less than about: 20, 30, 40, 50 amino acids in length. A fragment may be less than about: 20, 30, 40, 50 oligonucleotides in length.

The term “homology,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=(# of identical positions/total # of positions)×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK). The term “length homology” can mean percent length identity between the length of a particular polypeptide and the length of another polypeptide. “Length homology” can in some instances be calculated by dividing the number of amino acids in a first peptide chain by the number of amino acids in a second peptide chain and multiplying the result by 100%. Chain amino acids can be those forming the backbone of the peptide.

The term “microenvironment,” as used herein, may refer to the tumor microenvironment as a whole or to an individual subset of cells within the microenvironment. Exemplary cells within the tumor microenvironment may include but not limited to Tumor Infiltrating Lymphocytes (TILs), leukocytes, macrophages, and/or other cells of the immune system, and/or stromal cells, and/or fibroblasts (e.g., cancer or tumor associated fibroblasts).

The phrase “pharmaceutically acceptable” may be employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein may refer to a pharmaceutically acceptable material, composition or vehicle, such as a liquid or solid filler, diluent, excipient, solvent or encapsulating material. Some examples of materials which may serve as pharmaceutically acceptable carriers may include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; or (21) other non-toxic compatible substances employed in pharmaceutical formulations.

In some embodiments, the term “prevent” or “preventing” as related to a disease or disorder may refer to a compound or composition that, in a statistical sample, may reduce the occurrence of the disorder or condition in the treated sample relative to an untreated control sample, or may delay the onset or may reduce the severity of one or more symptoms of the disorder or condition relative to the untreated control sample.

The terms “treat,” “treating” or “treatment,” as used herein, may include at least partially: alleviating, abating or ameliorating a disease or condition symptom; preventing an additional symptom; ameliorating or preventing the underlying causes of symptom; inhibiting the disease or condition, e.g., at least partially arresting the development of the disease or condition; relieving the disease or condition; causing regression of the disease or condition; relieving a condition caused by the disease or condition; or stopping a symptom of the disease or condition either prophylactically and/or therapeutically. Treatment may include stopping the growth of a cancer, shrinking the mass of a cancer, slowing the growth of a cancer shrinking the volume of a cancer, or prolonging the life span of a subject when compared to an otherwise substantially identical subject who may not be treated.

Included in the present disclosure may be salts, including pharmaceutically acceptable salts, of the compositions described herein. The compounds or compositions of the present disclosure that may possess a sufficiently acidic, a sufficiently basic, or both functional groups, may react with any of a number of inorganic bases, inorganic acids, or organic acids, to form a salt. Alternatively, compositions containing compounds that are inherently charged, such as those with quaternary nitrogen, may form a salt with an appropriate counterion, e.g., a halide such as bromide, chloride, or fluoride, particularly bromide.

As used herein, “agent” or “biologically active agent” may refer to a biological, pharmaceutical, or chemical compound or a salt of any of these. Non-limiting examples may include a simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds may be synthesized, for example, small molecules and oligomers (e.g., oligopeptides and oligonucleotides), or synthetic organic compounds based on various core structures. In addition, various natural sources may provide compounds for screening, such as plant or animal extracts, and the like.

As used herein, an “immunotherapeutic agent” may refer to an agent that may be used on or used to modify an immune mechanism or immune response.

As used herein, a “pharmaceutical agent” may refer to an agent or a therapy that may be used to prevent, diagnose, treat, or cure a disease, or combinations thereof.

Activity of a protein, as used herein, may refer to a transcript level of mRNA transcribed from a gene that codes for said protein, expression level of said protein, nature of the expressed protein, such as folding, ability of said protein to interact with other proteins in the tumor microenvironment, and ability of said protein to interact with downstream or upstream signaling molecules in a signaling cascade of which said protein is a member.

The methods and compositions described herein include the use of amorphous forms as well as crystalline forms or polymorphs. The compounds described herein may be in the form of pharmaceutically acceptable salts. As well, active metabolites of these compounds having the same type of activity may be included in the scope of the present disclosure. In addition, the compounds described herein may exist in unsolvated as well as solvated forms with pharmaceutically acceptable solvents such as water, ethanol, and the like. The solvated forms of the compounds presented herein may also considered to be disclosed herein.

As used herein, a “biosimilar” or a “biosimilar product” may refer to a biological product that is licensed based on a showing that it is highly similar to an FDA-approved biological product, known as a reference product, and has no clinically meaningful differences in terms of safety and effectiveness from the reference product. Only minor differences in clinically inactive components may be allowable in biosimilar products. A “biosimilar” of an approved reference product/biological drug refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biological product. In some embodiments, the biosimilar biological product and reference product utilize the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. In some embodiments, the facility in which the biological product is manufactured, processed, packed, or held may meet standards designed to assure that the biological product continues to be safe, pure, and potent. The reference product may be approved in at least one of the U.S., Europe, or Japan. In some embodiments, a response rate of human subjects administered the biosimilar product can be 50%-150% of the response rate of human subjects administered the reference product. For example, the response rate of human subjects administered the biosimilar product can be 50%-100%, 50%-110%, 50%-120%, 50%-130%, 50%-140%, 50%-150%, 60%-100%, 60%-110%, 60%-120%, 60%-130%, 60%-140%, 60%-150%, 70%-100%, 70%-110%, 70%-120%, 70%-130%, 70%-140%, 70%-150%, 80%-100%, 80%-110%, 80%-120%, 80%-130%, 80%-140%, 80%-150%, 90%-100%, 90%-110%, 90%-120%, 90%-130%, 90%-140%, 90%-150%, 100%-110%, 100%-120%, 100%-130%, 100%-140%, 100%-150%, 110%-120%, 110%-130%, 110%-140%, 110%-150%, 120%-130%, 120%-140%, 120%-150%, 130%-140%, 130%-150%, or 140%-150% of the response rate of human subjects administered the reference product. In some embodiments, a biosimilar product and a reference product can utilize the same mechanism or mechanisms of action for the condition or conditions of use prescribed, recommended, or suggested in the proposed labeling, but only to extent the mechanism or mechanisms are known for the reference product.

A polypeptide disclosed herein may have one or more modifications, such as a post-translational modification (e.g., glycosylation, phosphorylation, etc.) or any other modification (e.g., pegylation, etc.). The polypeptide may contain one or more non-naturally-occurring amino acids (e.g., such as an amino acid with a side chain modification).

To obtain approval for biosimilar drugs, studies and data of structure, function, animal toxicity, pharmacokinetics, pharmacodynamics, immunogenicity, and clinical safety and efficacy may be needed.

A biosimilar may also be known as a follow-on biologic or a subsequent entry biologic. In some embodiments, a biosimilar product may be highly similar to the reference product notwithstanding minor different in clinically inactive components.

The expression construct for a proposed product may encode the same primary amino acid sequence as its reference product. Minor modifications may be present. Minor modifications may include N- or C-terminal truncations or alterations.

In some embodiments, a method described herein may comprise administering a therapeutically effective amount of an antibody or a fragment thereof.

As used herein, a “interchangeable biological product” may refer to biosimilar to an FDA-approved reference product and meets additional standards for interchangeability. In some embodiments, an interchangeable biological product can, for example, produce the same clinical result as the reference product in any given patient. In some embodiments, an interchangeable product may contain the same amount of the same active ingredients, may possess comparable pharmacokinetic properties, may have the same clinically significant characteristics, and may be administered in the same way as the reference compound. In some embodiments, an interchangeable product can be a biosimilar product that meets additional standards for interchangeability. In some embodiments, an interchangeable product can produce the same clinical result as a reference product in all of the reference product's licensed conditions of use. In some embodiments, an interchangeable product can be substituted for the reference product by a pharmacist without the intervention of the health care provider who prescribed the reference product. In some embodiments, when administered more than once to an individual, the risk in terms of safety or diminished efficacy of alternating or switching between use of the biological product and the reference product is not greater than the risk of using the reference product without such alternation or switch. In some embodiments, an interchangeable product can be a regulatory agency approved product. In some embodiments, a response rate of human subjects administered the interchangeable product can be 80%-120% of the response rate of human subjects administered the reference product. For example, the response rate of human subjects administered the interchangeable product can be 80%-100%, 80%-110%, 80%-120%, 90%-100%, 90%-110%, 90%-120%, 100%-110%, 100%-120%, or 110%-120 of the response rate of human subjects administered the reference product.

In some embodiments, a method of the current disclosure comprises administering a therapeutically effective amount of a diagnostic to a patient who has been administered an FDA approved diagnostic.

### Methods

In some embodiments, a method of the current disclosure may comprise administering a therapeutically effective amount of an immunotherapeutic agent or a salt thereof, wherein the immunotherapeutic agent or a salt thereof may inhibit a biological cascade selected from the group consisting of: a B-cell activating factor receptor cascade, a transmembrane activator and calcium-modulator and cyclophilin ligand interactor receptor cascade, a B-cell maturation antigen receptor cascade, and any combination thereof; wherein the immunotherapeutic agent may be co-administered with a checkpoint inhibitor.

In some embodiments, a method of the current disclosure may comprise treating a subject in need thereof, comprising: administering a therapeutically effective amount of an immunotherapeutic agent or a salt thereof, wherein the immunotherapeutic agent or a salt thereof may inhibit a biological cascade selected from the group consisting of: a B-cell activating factor receptor cascade, a transmembrane activator, calcium-modulator, cyclophilin ligand interactor receptor cascade, a B-cell maturation antigen receptor cascade, and any combination thereof, and administering a checkpoint inhibitor.

In some embodiments, a method of treatment of the current disclosure may result in at least partial remission of a cancer or a tumor. After administration of a composition described herein, progression of a cancer or a tumor may be delayed. Methods described herein may delay progression of a cancer or a tumor. After administration of a composition, the mass of a cancer or a tumor may be reduced, or the volume of a cancer or a tumor may be reduced. A method described herein may prolong the lifespan, the expected lifespan, or the life of a subject.

In one aspect, the present disclosure provides a method of treating a subject in need thereof. The method may comprise: administering a therapeutically effective amount of at least one B-cell activating factor (BAFF) inhibitor and/or at least one B-cell activating factor receptor (BAFF-R) inhibitor, or a salt thereof; and administering a therapeutically effective amount of at least one checkpoint inhibitor or a salt thereof. The subject may have been diagnosed with a cancer or a tumor. In some embodiments, the method may be a method of treating a cancer or a tumor in the subject. The subject may not have previously undergone treatment for the cancer or the tumor. The subject may have previously or may be currently undergoing treatment for the cancer or the tumor. The subject may have previously undergone treatment with surgery or radiation, and the subject remains in remission. The subject may have previously undergone treatment with surgery, radiation, an anticancer agent, and any combination thereof, and the cancer or the tumor may have been at least partially refractive to the treatment. The subject may have been previously treated with surgery, radiation therapy, an anticancer agent, and any combination thereof, and the cancer or the tumor may have re-occurred in the subject following a period of remission, wherein the period of remission may be at least about: 1 month, 2 months, 3 months, 6 months, 1 year, 2 years, 3 years, 4 years, 5 years, 6 years, 7 years, 8 years, 9 years, or 10 years.

In some embodiments, the subject may have been previously treated with a checkpoint inhibitor or a salt thereof, and the cancer or the tumor may have been at least partially refractive to the checkpoint inhibitor or a salt thereof. The subject may have been previously treated with a checkpoint inhibitor or a salt thereof, and the cancer or the tumor may have re-occurred in the subject following a period of remission, wherein a period of the remission may have been at least about: 1 month, 2 months, 3 months, 6 months, 1 year, 2 years, 3 years, 4 years, 5 years, 6 years, 7 years, 8 years, 9 years, or 10 years. The checkpoint inhibitor or a salt thereof may be administered sequentially with the BAFF inhibitor and/or BAFF-R inhibitor or a salt thereof. The checkpoint inhibitor or a salt thereof may be administered concurrently with the BAFF inhibitor and/or BAFF-R inhibitor or a salt thereof. The checkpoint inhibitor may be selected from the group consisting of an agent that binds to anti-programmed cell death protein 1 (PD-1), anti-programmed death ligand 1 (PD-L1), anti-programmed death ligand 2 (PD-L2), cytotoxic T-lymphocyte-associated protein 4 (CTLA4), cluster of differentiation 276 (B7-H3), V-set domain-containing T-cell activation inhibitor 1 (B7-H4), T-cell immunoglobulin and mucin-domain containing-3 (TIM-3), lymphocyte-activation gene 3 (LAG3), Indoleamine-pyrrole 2,3-dioxygenase (IDO), a salt of any one thereof, and any combination thereof. In some embodiments, a composition disclosed herein can be administered with IL-6 or an antihistamine. In some embodiments, a compositions disclosed herein can be administered with a third agent. In some embodiment, a third agent can be administered together with a composition disclosed herein, subsequently, or before a composition disclosed herein.

In some embodiments, the BAFF-inhibitor may comprise the sequence of any of SEQ ID Nos. 1-16, 150-163, a fragment of any of SEQ ID Nos. 1-19, 150-163 or a sequence having at least about 50%, 55%, 60%, 65%, 70%, 75%, 80%, 85%, 90%, 95%, 96%, 97%, 98%, 99%, or more sequence homology to any of SEQ ID Nos. 1-19, 150-163. In some embodiments, the BAFF-R inhibitor may comprise the sequence of any of SEQ ID Nos. 21-30, 74-85, a fragment of any of SEQ ID Nos. 21-30, 84, 85, or a sequence having at least about 50%, 55%, 60%, 65%, 70%, 75%, 80%, 85%, 90%, 95%, 96%, 97%, 98%, 99%, or more sequence homology to any of SEQ ID Nos. 21-30, 74-85. In some embodiments, the checkpoint inhibitor may comprise the sequence of any of SEQ ID NOs: 17-20, 165-174 (see Table 3B), a fragment of any of SEQ ID Nos: 17-20, 165-174 a sequence having at least about: 50%, 55%, 60%, 65%, 70%, 75%, 80%, 85%, 90%, 95%, 96%, 97%, 98%, 99%, or more sequence homology to a sequence of any of SEQ ID Nos: 17-20, 165-174 or any combination thereof.

In some embodiments, the BAFF-R inhibitor is VAY736. VAY736 is a fully human combinatorial antibody library (HuCAL)-derived monoclonal antibody targeting BAFF-R. In some examples, the heavy chain CDR1 of VAY736 can have the amino acid sequence of GDSVSSNSAAWG (SEQ ID NO: 74). The heavy chain CDR2 of VAY736 can have the amino acid sequence of RIYYRSKWYNSYAVSVKS (SEQ ID NO: 75). The heavy chain CDR3 of VAY736 can have the amino acid sequence of YDWVPKIGVFDS (SEQ ID NO: 76). The light chain CDR1 of VAY736 can have the amino acid sequence of RASQFISSSYLS (SEQ ID NO: 77). The light chain CDR2 of VAY736 can have the amino acid sequence of LLIYGSSSRAT (SEQ ID NO: 78). The light chain CDR3 of VAY736 can comprise the amino acid sequence of QQLYSSPM (SEQ ID NO: 79). The heavy chain variable region of VAY736 has the amino acid sequence of: QVQLQQSGPGLVKPSQTLSLTCAISGDSVSSNSAAWGWIRQSPGRGLEWLGRIYYRSKW YNSYAVSVKSRITINPDTSKNQFSLQLNSVTPEDTAVYYCARYDWVPKIGVFDSWGQGT LVTVSS (SEQ ID NO: 80). The light chain variable region of VAY736 has the amino acid sequence of: DIVLTQSPATLSLSPGERATLSCRASQFISSSYLSWYQQKPGQAPRLLIYGSSSRATGVPA RFSG SGSGTDFTLTISSLEPEDFAVYYCQQLYSSPMTFGQGTKVEIKRT (SEQ ID NO: 81). The heavy chain of VAY736 has the amino acid sequence of: QVQLQQSGPGLVKPSQTLSLTCAISGDSVSSNSAAWGWIRQSPGRGLEWLGRIYYRSKW YNSYAVSVKSRITINPDTSKNQFSLQLNSVTPEDTAVYYCARYDWVPKIGVFDWGQGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKRVEPKSCDKTHTCPPCPAP ELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVY TLPPSREEMTKNQVSLTCLVKGFYPSDIAVEWESNGQPENNYKTTPPVLDSDGSFFLYSK LTVDKSRWQQGNVFSCSVM HEALHNHYTQKSLSLSPGK (SEQ ID NO: 82). The light chain variable region of VAY736 can have the amino acid sequence of: DIVLTQSPATLSLSPGERATLSCRASQFISSSYLSWYQQKPGQAPRLLIYGSSSRATGVPA RFSGSGSGTDFTLTISSLEPEDFAVYYCQQLYSSPMTFGQGTKVEIKRTVAAPSVFIFPPSD EQLKSGTASVVCLLNNFYPREAKVQWKVDNALQSGNSQESVTEQDSKDSTYSLSSTLTL SKADYEKHK VYACEVTHQGLSSPVTKSFNRGEC (SEQ ID NO: 83).

In some embodiments, the BAFF inhibitor can comprise the entire length or a fraction of any one of the sequences SEQ ID Nos. 1-16 or 150-163. The BAFF inhibitor, in some examples, can comprise a sequence that can be about 50% to about 99% homologous to the entire length of or a fraction of the entire length of any of SEQ ID Nos. 1-16 or 150-163. In some cases, the BAFF inhibitor can comprise a sequence that can be about 50%, about 55%, about 60%, about 65%, about 70%, about 75%, about 80%, about 85%, about 90%, about 95%, about 96%, about 97%, about 98%, or about 99% homologous to the entire length of or a fraction of the entire length of any of SEQ ID Nos. 1-16 or 150-163.

A BAFF inhibitor may comprise an antibody, a small molecule, a protein, a peptide, an aptamer, an enzyme, or any combination thereof. In some embodiments, A BAFF inhibitor may comprise an antibody, an antigen binding fragment thereof, a BAFF binding domain, or any combination thereof. The BAFF inhibitor may comprise a mouse antibody. The BAFF inhibitor may comprise a rat antibody. The BAFF inhibitor may comprise a human antibody. The BAFF inhibitor may comprise a humanized antibody. The BAFF inhibitor may comprise a light chain region, a heavy chain region, or a combination thereof. The light chain region may comprise any one of CDR1, CDR2, CDR3, or any combination thereof. The heaving chain may comprise any one of CDR1, CDR2, CDR3, or any combination thereof. The BAFF inhibitor may comprise one or more antibody framework regions. In some embodiments, one or more of the framework regions may be humanized.

A BAFF inhibitor may comprise a sequence having at least about: 80%, 85%, 90%, 95%, or 99% sequence homology to a light chain CDR1, light chain CDR2, light chain CDR3, heavy chain CDR1, heavy chain CDR2, heavy chain CDR3, or any combination thereof of one of the following: tabalumab, atacicept, BAFF/IL-17 bispecific antibody, ardenermin, briobacept, blisibimod, or belimumab.

A BAFF inhibitor may comprise a sequence having at least about: 80%, 85%, 90%, 95%, or 99% sequence homology to at least a portion of a variable region of one of the following: tabalumab, atacicept, BAFF/IL-17 bispecific antibody, ardenermin, briobacept, blisibimod, or belimumab.

A BAFF inhibitor may comprise a sequence having at least about: 80%, 85%, 90%, 95%, or 99% sequence homology to at least a portion of an antibody binding domain of one of the following: tabalumab, atacicept, BAFF/IL-17 bispecific antibody, ardenermin, briobacept, blisibimod, or belimumab.

A BAFF, BLyS, TALL-1, THANK, neutrokine-α, or zTNF inhibitor may comprise a light chain sequence, a heavy chain sequence, wherein the light chain comprises CDR1, 2, and 3 and the heavy chain region also comprises CDR1, CDR2, CDR3. Exemplary heavy chain, light chain, and CDR sequences are provided in Table 1. Table 1B further comprises sequences of anti-BAFF-R antibodies. Contemplated are sequences that have about 80%, 85%, 90%, 96%, 97%, 98%, 99%, or more homology to heavy chain, light chain, and CDR sequences of Table 1 or Table 1B.

TABLE 1

| SEQ ID NO | SEQUENCE |
|---|---|
| 1: exemplary | GAAATTGTGTTGACGCAGTCTCCAGCCACCCTGTCTTTGTCTCCAGG |
| polynucleotide | GGAAAGAGCCACCCTCTCCTGCAGGGCCAGTCAGAGTGTTAGCCGCTACTTAGCCT |
| sequence | GGTACCAGCAGAAACCTGGCCAGGCTCCCAGGCTCCTCATCTATGATGCATCCAAC |
| encoding light | AGGGCCACTGGCATCCCAGCCAGGTTCAGTGGCAGTGGGTCTGGGACAGACTCCAC |
| chain variable | TCTCACCATCAGCAGCCTAGAGCCTGAAGATTTTGCAGTTTATTACTGTCAGCAGCG |
| region | TAGCAACTGGCCTCGGACGTTCGGC CAAGGGACCAAGGTGGAAATCAAACGAACT |
| 2: exemplary | EIVLTQSPATLSLSPGERATLSCRASQSVSRYLAWYQQKPGQAPRLLIYDASNRATGIPA |
| amino acid | RFSGSGSGTDSTLTISSLEPEDFAVYYCQQRSNWPRTFGQGTKVEIKRT |
| light chai | |
| sequence nor | |
| variable region | |
| 3: exemplary | AGGGCCAGTCAGAGTGTTAGCCGCTACTTAGCC |
| polynucleotide | |
| sequence for | |
| light chain CDR1 | |
| 4: exemplary | RASQSVSRYLA |
| amino acid | |
| sequence for | |
| light chain CDR1 | |
| 5: exemplary | GATGCATCCAACAGGGCCACT |
| polynucleotide | |
| sequence for | |
| light chain CDR2 | |
| 6: exemplary | DASNRAT |
| amino acid | |
| sequence for | |
| light chain CDR2 | |
| 7: exemplary | CAGCAGCGTAGCAACTGGCCTCGGACG |
| polynucleotide | |
| sequence for | |
| light chain CDR3 | |
| 8: exemplary | QQRSNWPRT |
| amino acid | |
| sequence for | |
| light chain CDR3 | |
| 9: exemplary | ATGAAACACCTGTGGTTCTTCCTCCTCCTGGTGGCAGCTCCCAGATGGGTCCTGTCC |
| polynucleotide | CAGGTGCAACTACAGCAGTGGGGCGCAGGACTGTTGAAGCCTTCGGAGACCCTGTC |
| sequence | CCTCACCTGCGCTGTCTATGGTGGGTCCTTCAGTGGTTACTACTGGAGCTGGATCCG |
| encoding heavy | CCAGCCCCCAGGGAAGGGGCTGGAGTGGATTGGGGAAATCAATCATAGTGGAAGC |
| chain variable | ACCAACTACAACCCGTCCCTCAAGAGTCGAGTCACCATATCAGTAGACACGTCCAA |
| region | GAACCAGTTCTCCCTGAAACTGAGCTCTGTGACCGCCGCGGACACGGCTGTGTATTA |
| | CTGTGCGAGAGGGTATTACGATATTTTGACTGGTTATTATTACTACTTTGACTACTG |
| | GGGCCAGGGAACCCTGGTCACCGTCTCCTCA |
| 10: exemplary | MKHLWFFLLLVAAPRWVLSQVQLQQWGAGLLKPSETLSLTCAVYGGSFSGYYWSWIR |
| amino acid | QPPGKGLEWIGEINHSGSTNYNPSLKSRVTISVDTSKNQFSLKLSSVTAADTAVYYCARG |
| sequence | YYDILTGYYYYFDYWGQGTLVTVSS |
| encoding heavy | |
| chain variable | |
| region | |
| 11: exemplary | GGTGGGTCCTTCAGTGGTTACTACTGGAGC |
| polynucleotide | |
| sequence | |
| encoding heavy | |
| chain CDR1 | |
| 12: exemplary | GGSFSGYYWS |
| amino acid | |
| sequence for | |
| heavy chain | |
| CDR1 | |
| 13: exemplary | GAAATCAATCATAGTGGAAGCACCAACTACAACCCGTCCCTCAAGAGT |
| polynucleotide | |
| sequence | |
| encoding heavy | |
| chain CDR2 | |
| 14: exemplary | EINHSGSTNYNPSLKS |
| amino acid | |
| sequence for | |
| heavy chain | |
| CDR2 | |
| 15: exemplary | GGGTATTACGATATTTTGACTGGTTATTATTACTACTTTGACTAC |
| polynucleotide | |
| sequence | |
| encoding heavy | |
| chain CDR3 | |
| 16: exemplary | GYYDILTGYYYYFDY |
| amino acid | |
| sequence for | |
| heavy chain | |
| CDR3 | |
| 150: exemplary | QVQLQQWGAGLLKPSETLSLTCAVYGGSFSGYYWSWIRQPPGKGLEWIGEINHSGSTN |
| amino acid | YNPSLKSRVTISVDTSKNQFSLKLSSVTAADTAVYYCARGYYDILTGYYYYFDYWGQG |
| sequence for | TLVTVSSASTKGPSVFPLAPCSRSTSESTAALGCLVKDYFPEPVTVSWNSGALTSGVHTF |
| heavy chain | PAVLQSSGLYSLSSVVTVPSSSLGTKTYTCNVDHKPSNTKVDKRVESKYGPPCPPCPAPE |
| | FLGGPSVFLFPPKPKDTLMISRTPEVTCVVVDVSQEDPEVQFNWYVDGVEVHNAKTKP |
| | REEQFNSTYRVVSVLTVLHQDWLNGKEYKCKVSNKGLPSSIEKTISKAKGQPREPQVYT |
| | LPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLGK |
| 151: exemplary | EIVLTQSPATLSLSPGERATLSCRASQSVSRYLAWYQQKPGQAPRLLIYDASNRATGIPA |
| amino acid | RFSGSGSGTDSTLTISSLEPEDFAVYYCQQRSNWPRTFGQGTKVEIKRTVAAPSVFIFPPS |
| sequence for | DEQLKSGTASVVCLLNNFYPREAKVQWKVDNALQSGNSQESVTEQDSKDSTYSLSNTL |
| light chain | TLSKADYEKHKVYACEVTHQGLSSPVTKSFNRGEC |
| 152: exemplary | MKHLWFFLLL VAAPRWVLSQ VQLQQWGAGL LKPSETLSLT CAVYGGSFSG |
| amino acid | YYWSWIRQPP GKGLEWIGEI NHSGSTNYNP SLKSRVTISV DTSKNQFSLK |
| sequence for VH | LSSVTAADTA VYYCARGYYD ILTGYYYYFD YWGQGTLVTV SS |
| 153: exemplary | EIVLTQSPAT LSLSPGERAT LSCRASQSVS RYLAWYQQKP GQAPRLLIYD |
| amino acid | ASNRATGIPA RFSGSGSGTD STLTISSLEP EDFAVYYCQQ RSNWPRTFGO |
| sequence for VL | GTKVEIKRT |
| 154: exemplary | QVQLQQSGAE VKKPGSSVRV SCKASGGTFN NNAINWVRQA PGQGLEWMGG I |
| amino acid | IPMFGTAKY SQNFQGRVAI TADESTGTAS MELSSLRSED TAVYYCARSR |
| sequence for | DLLLFPHHAL SPWGRGTMVT VSSASTKGPS VFPLAPSSKS TSGGTAALGC |
| heavy chain | LVKDYFPEPV TVS NSGALT SGVHTFPAVLQSSGLYSLSS VVTVPSSSLG |
| | TQTYICNVNH KPSNTKVDKK VEPKSCDKTH TCPPCPAPELLGGPSVFLFP |
| | PKPKDTLMI S RTPEVTCVVV DVSHEDPEVK FNWYVDGVEV HNAKTKPREE |
| | QYNSTYRVVS VLTVLHQDWL NGKEYKCKVS NKALPAPIEK TI SKAKGQPR |
| | EPQVYTLPPSRDELTKNQVS LTCLVKGFYP SDIAVEWESN GQPENNYKTT |
| | PPVLDSDGSF FLYSKLTVDKSRWQQGNVFS CSVMHEALHN HYTQKSLSLS PGK |
| 155: exemplary | SSELTQDPAV SVALGQTVRV TCQGDSLRSY YASWYQQKPG QAPVLVIYGK |
| amino acid | NNRPSGI PDR FSGSSSGNTA SLTITGAQAE DEADYYCSSR DSSGNHWVFG |
| sequence for | GGIELTVLGQ PKAAPSVTLF PPSSEELQAN KATLVCLISD FYPGAVTVAW KADSSPV |
| light chain | AG VETTTPSKQS NNKYAASSYL SLTPEQWKSH RSYSCQVTHE GSTVEKTVAP TECS |
| 156: exemplary | QVQLQQSGAE VKKPGSSVRV SCKASGG FN NNAINWVRQA PGQGLE GG I IP |
| amino acid | FGTAKY SQNFQGRV I TADESTGTAS MELSSLRSED TAVYYCARSR DLLLFPHHAL |
| sequence for VH | SPWGRGTMVT VSS |
| 157: exemplary | SSELTQDPAV SVALGQTVRV TCQGDSLRSY YASWYQQKPG QAPVLVIYGK NNRP |
| amino acid | FSGSSSGNTA SLTITGAQAE DEADYYCSSR DSSGNHWVFG GGTELTVLGGTKVEIKRT |
| sequence for VL | |
| 158: exemplary | GGTFNNNAIN |
| amino acid | |
| sequence for | |
| CDRH1 | |
| 159: exemplary | GIIPMFGTAK YSQNFQG |
| amino acid | |
| sequence for | |
| CDRH2 | |
| 160: exemplary | SRDLLLFPHH ALSP |
| amino acid | |
| sequence for | |
| CDRH3 | |
| 161: exemplary | QGDSLRSYYA S |
| amino acid | |
| sequence for | |
| CDRL1 | |
| 162: exemplary | GKNNRPS |
| amino acid | |
| sequence for | |
| CDRL2 | |
| 163: exemplary | SSRDSSGNHW V |
| amino acid | |
| sequence for | |
| CDRL3 | |

In some embodiments, a sequence of a composition described herein may comprise at least about: 100% homology, 95% homology, 90% homology, 85% homology, 80% homology, 75% homology, 70% homology, 65% homology, 60% homology, 55% homology, 50% homology, to an entire length or a fraction of an entire length of a reference sequence, or a sequence listed in Table 1 or Table 1B.

TABLE 1B

| Seq ID NO: | Sequence |
|---|---|
| 21: exemplary | GDSVSSNSAAWG |
| heavy chain | |
| CDR1 | |
| 22: exemplary | RIYYRSKWYNSYAVSVKS |
| heavy chain | |
| CDR2 | |
| 23: exemplary | YDWVPKIGVFDS |
| heavy chain | |
| CDR3 | |
| 24: exemplary | RASQFISSSYLS |
| light chain CDR1 | |
| 25: exemplary | LLIYGSSSRAT |
| light chain CDR2 | |
| 26: exemplary | QQLYSSPM |
| light chain CDR3 | |
| 27: exemplary | QVQLQQSGPGLVKPSQTLSLTCAISGDSVSSNSAAWGWIRQSPGRGLEWLGRIYYRSK |
| heavy chain | WYNSYAVSVKSRITINPDTSKNQFSLQLNSVTPEDTAVYYCARYDWVPKIGVFDSWGQ |
| variable region | GTLVTVSS |
| 28: exemplary | DIVLTQSPATLSLSPGERATLSCRASQFISSSYLSWYQQKPGQAPRLLIYGSSSRATGVPA |
| light chain | RFSGSGSGTDFTLTISSLEPEDFAVYYCQQLYSSPMTFGQGTKVEIK |
| variable region | |
| 29: exemplary | QVQLQQSGPGLVKPSQTLSLTCAISGDSVSSNSAAWGWIRQSPGRGLEWLGRIYYRSK |
| heavy chain | WYNSYAVSVKSRITINPDTSKNQFSLQLNSVTPEDTAVYYCARYDWVPKIGVFDSWGQ |
| | GTLVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKRVEPKSCDKTHTCPP |
| | CPAPELLGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| 30: exemplary | DIVLTQSPATLSLSPGERATLSCRASQFISSSYLSWYQQKPGQAPRLLIYGSSSRATGVPA |
| light chain | RFSGSGSGTDFTLTISSLEPEDFAVYYCQQLYSSPMTFGQGTKVEIKRTVAAPSVFIFPPS |
| variable region | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC |
| 84: exemplary | GSSSRAT |
| light chain CDR2 | |
| 85: exemplary | QQLYSSPMT |
| light chain CDR3 | |

In some embodiments, the BAFF or a BAFF-R inhibitor can comprise an aptamer comprising 100% homology, 95% homology, 90% homology, 85% homology, 80% homology, 75% homology, 70% homology, 65% homology, 60% homology, 55% homology, 50% homology, to an entire length or a fraction of an entire length of Aptamer 1 (SEQ ID NO: 175) 5′-GGG AGG ACG AUG CGG GAG GCU CAA CAA UGA UAG AGC CCG CAA UGU UGA UAG UUG UGC CCA GUC UGC AGA CGA CUC GCC CGA-3; Aptamer 2 (SEQ ID NO: 176) 5′-GGG AGG ACG AUG CGG AUA ACU AUU GUG CUA GAG GGC UUA UUU AUG UGA GCC GGU UGA UAG UUG CGC AGA CGA CUC GCC CGA-3; or Aptamer 3 (SEQ ID NO: 177) 5′-GGG AGG ACG AUG CGG AUC CUC CGA AGG UCG CGC CAA CGU CAC ACA UUA AGC UUU UGU UCG UCU GCA GAC GAC UCG CCC GA-3′.

In some cases, a BAFF-R protein can comprise an amino acid sequence substantially the same as described in Table 1C.

TABLE 1C

| SEQ ID NO | SEQUENCE |
|---|---|
| 164. Human | MRRGPRSLRGRDAPAPTPCVPAECFDLLVRHCVAC |
| BAFFR amino | GLLRTPRPKPAGASSPAPRTALQPQESVGAGAGEA |
| acid sequence | ALPLPGLLFGAPALLGLALVLALVLVGLVSWRRRQ |
| | RRLRGASSAEAPDGDKDAPEPLDKVIILSPGISDA |
| | TAPAWPPPGEDPGTTPPGHSVPVPATELGSTELVT |
| | TKTAGPEQQ |

In some cases, a BAFF protein can comprise an amino acid sequence substantially the same as described in Table 2.

TABLE 2

| SEQ ID NO | SEQUENCE |
|---|---|
| 31: Polypeptide | MDDSTEREQSRLTSCLKKREEMKLKECVSILPRKESPSVRSSKDGKLLAATLLLALLSCC |
| sequence of | LTVVSFYQVAALQGDLASLRAELQGHHAEKLPAGAGAPKAGLEEAPAVTAGLKIFEPP |
| BAFF Isoform 1 | APGEGNSSQNSRNKRAVQGPEETVTQDCLQLIADSETPTIQKGSYTFVPWLLSFKRGSAL |
| | EEKENKILVKETGYFFIYGQVLYTDKTYAMGHLIQRKKVHVFGDELSLVTLFRCIQNMP |
| | ETLPNNSCYSAGIAKLEEGDELQLAIPRENAQISLDGDVTFFGALKLL |
| 32: Polypeptide | MDDSTEREQSRLTSCLKKREEMKLKECVSILPRKESPSVRSSKDGKLLAATLLLALLSCC |
| sequence of | LTVVSFYQVAALQGDLASLRAELQGHHAEKLPAGAGAPKAGLEEAPAVTAGLKIFEPP |
| BAFF Isoform 2 | APGEGNSSQNSRNKRAVQGPEETGSYTFVPWLLSFKRGSALEEKENKILVKETGYFFIY |
| | GQVLYTDKTYAMGHLIQRKKVHVFGDELSLVTLFRCIQNMPETLPNNSCYSAGIAKLEE |
| | GDELQLAIPRENAQISLDGDVTFFGALKLL |
| 33: Polypeptide | MDDSTEREQSRLTSCLKKREEMKLKECVSILPRKESPSVRSSKDGKLLAATLLLALLSCC |
| sequence of | LTVVSFYQVAALQGDLASLRAELQGHHAEKLPAGAGAPKAGLEEAPAVTAGLKIFEPP |
| BAFF Isoform 3 | APGEGNSSQNSRNKRAVQGPEETVTQDCLQLIADSETPTIQKGFIY |
| 34: Polypeptide | MDDSTEREQSRLTSCLKKREEMKLKECVSILPRKESPSVRSSKDGKLLAATLLLALLSCC |
| sequence of | LTVVSFYQVAALQGDLASLRAELQGHHAEKLPAGAGAPKAGLEETPAVTAGLKIFEPP |
| BAFF Isoform 1 | APGEGNSSQNSRNKRAVQGPEETVTQDCLQLIADSETPTIQKGSYTFVPWLLSFKRGSAL |
| Variant | EEKENKILVKETGYFFIYGQVLYTDKTYAMGHLIQRKKVHVFGDELSLVTLFRCIQNMP |
| | ETLPNNSCYSAGIAKLEEGDELQLAIPRENAQISLDGDVTFFGALKLL |
| 149: Polypeptide | AVQGPEETVTQDCLQLIADS ETPTIQKGSY TFVPWLLSFK RGSALEEKEN |
| BAFF soluble | KILVKETGYF FIYGQVLYTD K Y MGHLIQ RKKVHVFGDE LSLVTLFRCI |
| sequence | QNMPETLPNN SCYSAGIAKL EEGDELQLAI PRE AQI SLD GDVTFFGALK LL |

In some embodiments, a BAFF protein described herein may comprise at least about: 100% identity, 95% identity, 90% identity, 85% identity, 80% identity, 75% identity, 70% identity, 65% identity, 60% identity, 55% identity, 50% identity, of a reference sequence, or a sequence listed in Table 2.

In some cases, a BAFF protein can be expressed from one or more coding regions from chromosome 13. In some cases, a BAFF protein can be expressed from one or more coding regions of the polynucleotide sequence of SEQ ID NO. 35 depicted in Table 3.

TABLE 3

| SEQ ID NO | SEQUENCE |
|---|---|
| 35: | AGACTAAAAAATGGGTTCAATGTGTACACTGCTCGGGTGATGGGTGCACCAAAATCTC |
| NC_00013.11 | ACAAATCACCACTAAATAACTTAACCATGTAATCAAATACCACCTATTCCCCAAAAAC |
| | CTGTGGAAATAATTTTTTTTTAAATGGAAGCATCCAAAATGTAATTTGTTTCCACATTC |
| | TTGTAAGCCTCATTATGTATCCCAGGGACAGTTAATATCGTCATACGAATTCACAGGT |
| | GAGGAAATGGAATCACAGAGAGATTATGTACTTTGCATAAGGAGAGCGAGGGTGAGG |
| | CAGGATTTGCAGTCTAGAAGCCTGGGTCTGGAGTTCTCCACTTTGCACTATATCATTTC |
| | TGTAAACTTCTTACTTAAGACTGTGTGGAAATGTAGAGTGAGACAGAACTAAAGCTCA |
| | CTATTCTTAGTCCACAGGGGAGTAGTGACTACCCAGGGCTTGGTAAGTGCTCAGTAAC |
| | GTTTGTTGAAAGATGAATCAATATTTCAATGCTGGGGCAAAGCAGTGAAAAACTGGG |
| | GAATGTCCAGGAACCAGCCATCAGGATAAGGCAGGACTAGAATGAATGCAACAGTCC |
| | TCACAGGTCCACCAAGTCAACAACAGAAAAATCCAAAGCTAAGGTGGCTCTTGTGTG |
| | ATCAAGGACCTCTTGTACACCGACCTGTTAGGCTGTGGCACAGTCAACATGGGAGTTG |
| | TAGACAAGCTACTTGAGAGTGGTTAATGGTAGCAGTCAGGCATTTGGAGCTGGAGGC |
| | AAGGCTGATTCTCCTCAACTAGTATCATATTGAGCGGGGACTTAAAGCCTCAGAAGTC |
| | TGAGCAGAAAAGCTGAGACCAGGCTCCTTGGGACAGGCTGTTCATATGCATGATTGAG |
| | TTTCAGTGATGAGTCCCAGAAGGAAAAAGTTTATGCATGTTTTCCTTAAAAATATATTC |
| | ACTTATATTTAGAGAATCCTTCAGATATCTAACCTAAATACGTCTGTAACATTTACTCT |
| | CATTCTTTTTATGACATTATTAGTATAATAATATGAATAATTCCCAGTGGACTTACTTC |
| | CACACTTCCTTCAAGGATTCTCCATTTTATTTCATAATTGAAGACTGTTAATAAATCAC |
| | TCTCTAGCTAAGAACAATTCAATGCTAAAACACTTAGCTTATCTGACCTTCACATTAAT |
| | TATTTTTATGACAGCAGCAGGAACTTTCAGCTGCTTTTCCCCCTTCCTTATGAAGCCAT |
| | GTGGTCCCAAGCAGACAGAGTTCCCTTGCTACACTTAAGGGTGGGCTTCTCAGACCTG |
| | AGCATATCTTAAAAGCTTTCCCTTGACTGTGCCAATCCAAACTCCAAGTTTCCTCAGCA |
| | GATTTAAGGGGTTTTAAATCTACTTGAGCATGAGTGTTATGAGAAGACTTTGAAATTC |
| | TTACAAAAACTGAAAGTGAAATGAGGAAGACAGATTGAGCAATCCAATCGGAGGGTA |
| | AATGCCAGCAAACCTACTGTACAGTAGGGGTAGAGATGCAGAAAGGCAGAAAGGAG |
| | AAAATTCAGGATAACTCTCCTGAGGGGTGAGCCAAGCCCTGCCATGTAGTGCACGCAG |
| | GACATCAACAAACACAGATAACAGGAAATGATCCATTCCCTGTGGTCACTTATTCTAA |
| | AGGCCCCAACCTTCAAAGTTCAAGTAGTGATATGGATGACTCCACAGAAAGGGAGCA |
| | GTCACGCCTTACTTCTTGCCTTAAGAAAAGAGAAGAAATGAAACTGAAGGAGTGTGTT |
| | TCCATCCTCCCACGGAAGGAAAGCCCCTCTGTCCGATCCTCCAAAGACGGAAAGCTGC |
| | TGGCTGCAACCTTGCTGCTGGCACTGCTGTCTTGCTGCCTCACGGTGGTGTCTTTCTAC |
| | CAGGTGGCCGCCCTGCAAGGGGACCTGGCCAGCCTCCGGGCAGAGCTGCAGGGCCAC |
| | CACGCGGAGAAGCTGCCAGCAGGAGCAGGAGCCCCCAAGGCCGGCCTGGAGGAAGCT |
| | CCAGCTGTCACCGCGGGACTGAAAGTGAGTTTGCAGCAGCTGCAAGACGCAGGCAAG |
| | ATCCTGCCTACACTGCTGCCTCTCCCTCGCCTCAGCTGTCTTTCTAATAACTTGAAGTTT |
| | TTCTGTTCATAGATCTTTGAACCACCAGCTCCAGGAGAAGGCAACTCCAGTCAGAACA |
| | GCAGAAATAAGCGTGCCGTTCAGGGTCCAGAAGAAACAGGTATGTTTTGGGCACAGA |
| | CACTTTTAGGAGTCAGGGATTAGAGAATAACATCCAGTCTGGGGGAGCTGGAGGTGA |
| | GTATCCCCTCTATGAACCTATTAAATAGAGACTATGAAATTTGCCATCCAAAGCAGAC |
| | ATTGCTTTAGAGCAAAGCCCCAGCGCTGGCAAATGTAACAGGCTGCATGGGTGTGCTT |
| | TTTCCTCCAATGAGCAGATCGTGTGACTGGAAGCATTTCTCTGTGCGTGTGTAAGTATT |
| | TATCTGTGCATTAAGCATAGAGGAGATAAAAGGGCCATATGTGTTTCCTCAGGTAGAT |
| | AATGCCCTCCCATGTGAGAAGCATTATTCATCACTCCAGAAAAATAAAAAAAAGCATT |
| | ACATGTTAGAATTAGACTTTGCTTACTACCTGGGTCATAGAAAGTTAAAGACATCTGT |
| | TTTCTGAATGTACATTACATGTTGTGAAACCAGGTGAAGGTTGTAATGAAAGGCCCTG |
| | TATGTTGTACTTTAATTTTGTTTATTTGTTAAAAAGGTTATTTTACACACACACACACA |
| | CACACGCACAGACACACACACAGATTTTAAAGCTTCTCTTTAAGGTACGCTTTTCCAG |
| | GGGATCCATGCATTTATCGTCTTCTTATAATCGTCCTTTACTGATTTCTGACTTTCTTCA |
| | TGTTCTGCTCATTCCACGGAACTTTACAGAGTTGGTGGGGCCTACCAAAATGATCGAT |
| | TCTAGTTCTTTCATTTGAAGGTGAAAAAAAGAATCTTTGTATACATTTATTAAAGTTAT |
| | AATATAACAAATTCTTAATATTATTGTCCTTTTATTAAATATATTGAAAGAATATTTTT |
| | TGAGCACCTGGAACGTATCGTACAGCTATAGACCAGAATAGGCATTTTCAAACATACT |
| | ATTTCGTTTAACCTTCACAGTGACGCTATGAAGTCCACAGAATACATGCAATAATTTAT |
| | GTATATTTTCACTTCTGAAGAGAATTGCATAAGAAATGTAGTGAGAGATAGACCAGGG |
| | ACCCTTCTATCACACACACACACACACACACACACACACACACACACACACACACGC |
| | ATGAGGTACCCTGACTATAAACTTATTTTTTGACTATATGCTTTATTCATAAAAAATTG |
| | TTTCTTATTTTATAAAACCATAATTTTTGGTATCTGGTTATTGTATCTTTATACTAGTTA |
| | TCAGGAATAACTGGGCTTAAACATTTTTAAATTAGGAAACATGCACATTTTTATTATTT |
| | TAACACATCAATTATTTTTATCGTCTGCATTTTATAGTCTATATTTCCTACATAATTGAA |
| | AATAATACGGAATATATACTATAATATTTTTTGTTTTGACTTATACAATAAGGATTTTT |
| | CCTATTTCTAAGTATTTTTCATATATCTGTATTAATTTATCTAACAATTGTTTTAAATGG |
| | TTATTTCCATTCTCTTTGCTATAATACAACATGCAAAAGTTTTGCACAGAACTCTTTTA |
| | TTTCTGTGAAATTATATATTCAATATAAAATTCATATAGTGGAATATCTGAGCTAGGG |
| | AGAAGAAACATCTTCATCACCCTTGCTTCCGACTGCCATATTGTTTTCAAAATGAGTTA |
| | TACATGCAATCAAAAATGTGGTGTACCGGTTCTCCACATTCTTGCTTGCATGTTATTTT |
| | TAAAGTGTATTTTGACTAGTTTAGTAGTTGTAAAACAATATCTCACATTTATTTTGATG |
| | TGTTGCTTGTTTACTAATATGAATAAACTTTCTGCATATTTTGATTATTTATAATTTTAT |
| | CTTTTGCTAATTTTTATTTATAACTGTCTCCACTTGTCTGTCCCATGTTTTTATAATTTTG |
| | GATAAGCTCTTCCTAATTAGACATATTGATAATTGATCAAAATTTTGATGTAAATATTT |
| | TAACAGTTTTGCTTCTTTTTTTACTTGAACATTTTGCATTTCTCTATAGTAAAGTGATAA |
| | TCTTTAAAAAATTTTCCCTAATATTAAAGCTAAAAATACATTTGTTCATGTTTTCTTAA |
| | GTAGTTGACTCCCTGCCACTTTGACTTTTTCATAGTTGAGTGTTTTGTTTTAACTCATTA |
| | GTCCTTCTGTTGTTTATTTTGATGAATCATCTCACATTGGCTTTAACTTCCCCCTCTCTC |
| | TTTTTTCCTTTCTAACTGGTATCTCAACCAAATGTATTAAATAGTTCTTTTTAACTCCTT |
| | GGTGTTGAAAAGCTTACTGTGTTTTATTAAGTTCCTCATAAGAGTTGAATCTATTTGAG |
| | ACTCTTTTTTTTAATTGATTATTCTATTTATATGCTTTAAAGAATATTTTTAAGGTAATT |
| | TGTATTATAAGAGGCTTCAATAACTAGTAGGGTCAGGATCCTTCAATCTCCTTAACCTT |
| | CACTGCTAATATCCCCGTTTCTAAATATTAGTTTTAAAAATAAACTTTAACTTTACAAT |
| | TAAAAAAAAAGATGTAGACTCATTTCAGCAGATTTAAGAAGAAAAGAATAATAGAGT |
| | AGGAAAGAAATAACTTCTTGGGTCACATTTTCCCAAAATTCTCCTATTAATACTTTTAT |
| | GTATTTGTGTTTTAATGGCATCCAGGAATAGTATACTGATGAGAATCTTTCACCAAATA |
| | ATTTGCAATTTGTAGATTCTAACAAAGTGAAAAAAAATTTGCCCAGATTTCCACAGCC |
| | CAGAAGTAGCTACTATTATTATTTCATGGTTTGCCCTAGACTTTTTAATAAACCTTAAT |
| | ACAGTGTGCATATAGGTATATATTGCACACAGTTGAGCCTTGAACAACACAAGTTTTA |
| | ACAGCATGGATACACTTACAGGTGGATTTTTTTTTTCAATAAACCTATTGGAAAATTAT |
| | CTGTAGATTTGTGATAATTTGAAAAAACTCATGGAAGAACTGTGTATCTTTGAAATGT |
| | TGAAAATATTAAGATTAATTAGCCATGTCATGAATGTATAAAATATATGTAGATACTG |
| | GTCTATTTTCTTATTACTATCATAAAATATACACAAAAGTTAAAAATTCTCAAAACATA |
| | TGCATACAAACAGACTGTACATAGCGCCCTTTGTAGTCTAGAGAAATGGAAACAAAC |
| | ATAAACATGCAGTATTAAGTCATCACTGCATAAAATTAACTGTGGTACATACTGTACT |
| | ACTGTAATAATTTCATAGCCACCTCCTGTTGCTACTGCAGTGAGCTCAAGTGCTGTGAG |
| | TATCTGCTTAAAACACCATGTGATGCTAATCATCTCTGCATGAGCCAGTTGTATCTCCA |
| | ATCAGTTGGATATTGCAGTAAAAATGATTTCTCATGATTTGTGTGTATTTTTCTTCTTGT |
| | GTAATGCAATATCATAAACCTTGAATAACACCATGAGACCTATAGAAAATGCGATAGT |
| | GATGCTGGAAATGCTCCCAGGAAGCAAAGAAGAGTCATGACATTATAGTTGACCCTTG |
| | AACAACACAGGTTTGAACCGCGAGGGTCCCCTTGTATGTGGATTTTCTTCCACTTTCCA |
| | CCCTTGAGATAGCAAGACCAACGCCTCCTTTCCCTCCTCCCCCTTAGCTTACTCAATGT |
| | GAAGAATGAAGAACTCTATAATGATCCACTTCCACTTAATGAATAGTAAATGCATTTT |
| | CTCTTCCTTATGATTTTTTAAAATAACATTTTCTTTTCTCTAGCTTATTTTATTGTAAGA |
| | ATACACTACATAATACATATAATTTATGAAATGCGTATTGATTGAGTTTTTGTTATCAG |
| | AAAGGCTTCTGGTCAACAGTGGGCTGTTAGTAATTAAGATTTGGGAGTCAAAAGTTAA |
| | GCACAAAGTTTTGACTGCATGGGGTCAGTGACCCTAATTCCTGTGTTGTTCAAGGATC |
| | AACTGTATTTGTTCATATACATATGAACAATACATATGAACAATACATATATGAACAA |
| | ATACATATATTTATACAAATATATGTACAAATATATACACATACAAATATATATATAT |
| | ATACATATATATACATACATAGTGTGCATATGTGTTATGTGTATATATTCAAATAGGAC |
| | ATAGTATACACACTCATAACTTTCACAAATCCAAGGTGGAAGATTTGAAGATAAGAAT |
| | CTTAATGACATCATGTCTACTATTCCTTGTTAATAAAATTAGGAAATTCATTTGAATGT |
| | TGTTCTAGATACTATTCTTTGGTGGAGGGAAAGTTTATTTTATTTAGCATACTCCAAAA |
| | TTAATGTTTATATTCCCAAAAAAGTTGTCTGTAACACAAGGACGTTTTTTAGTAGGAAT |
| | TTTTTGGCATTGAGAAAATTCTATGCAGCTTTCTTTGATTTTTGTTTTCATTCAAGTTCT |
| | GATTTATTTTCAGTAAGACTCAAAACCTTAAGTTGTCCTTTTTACTTTTAAAGTGTTTAT |
| | TTATGTTCATATTTATTAATTGTCTACATGTTTTAGAACCAAGTATTTTTATTGGAACTT |
| | CACACAAACTGTAGAAGAAGGGAGGGGAATTACCATCAGCCAACATGTCCCAATGAT |
| | ACTCTCTTCCAGGCATCCCATGGGTAAACAGTGGTTTTACTTAATGTTTTAATTTTAGA |
| | ATTTAGTACTTTGGCTTTAGTTTTGTTTTTCTTAATTACTTTGTGTTTCAAACCAAAATC |
| | TCATTAAGCTTAATGTATTTCATAATATCATTTTTGATTGTAGTATAATAAACAGAAAT |
| | GTGGGGAAATCCAGGATTAAAAAATTGCCAAATAGCTCATATTAGCCAAAAATTCACT |
| | TTTAAGCCCGTAACTGTGTTTGGAGATATTCCCAATTTAAAAATTAACTAAAACAATA |
| | TGGCTTTGAAAATATTACGTAATAAAATTTATTTACAAATGGAATCATGAAGATCCAC |
| | ATTTTGAACATCACTTAACTATAATTTTATTAATTTTAAGAACCTAAAGATGAATGTCT |
| | TTAGAGTCTATCAAGAAAGTGAATTCTTTCAAATGGGTATCACTGACCTAAAAAGGTT |
| | CATTGTTAAAATTAGGTAACCATTGTATTTTCTTTCCTGTTAATTAATAACTATTAGAA |
| | ATTTATGTGTTTTAGGATACAGAAAGACAAAATTATTTTTATTGATAATGTTTATTATA |
| | ATTGTCTCAATTTTAATAATAAAATAATAGTAAAAATATGGACAATTCAAAGTAATGT |
| | AAAGCAAGAATAGAAAAAATTGTCCGTATTTCACCTTCCAAATAATTATCATGAGATT |
| | TTTGGTCAATCTACTTTAAGGTATATTTTTCTTCATTTTTAAGTGAGCTTGGGATTGTTT |
| | AAGAATTACACACAGCTTGCAAAGAATACAGTGTGTTGGCATGTACCCCTATCCAGCT |
| | TCCCTAATACTAACATCTTATATGAACATGGTATTTCATTACAAAAAGGAAACCAACA |
| | TTAAGACTTAACTATTAGCTAAATTCTAGACTTAATTTGAATTTCACGAGTTTTTCTAC |
| | TAATGATTTTATGTTTTAGGATCCATCAGGATATCACATTGTATTTAACATTTTTTAAA |
| | TTAAAATTTTGAATGTAGCATATATAAACACCAAATATTATACCTTAAGTTTTCTATCG |
| | GATAAACCTTTCTAATTGCTAAAAGTGAAGATATCAATACTATTTTTGAGTCTTCTTTT |
| | TTGAAATCCAAAGTCCAACTCCTTTGCAAGGAAAGATGAGATGTTTGACAAGTCTGTA |
| | ATGTCCAGTCTTTGCCCGGTTGGCATTTCTGGCTTAGCTGTGAGTTCCAGGCTGTAACA |
| | TGAGTGTTGGAAGCGTGCAATGTAGAACTTGACCATCCAGCCCTTATCAAAATTAGGC |
| | TGACTTGAGCCTCTTACCCAGCAAGGGTGTGTTCCTTCGTGGAACAGAACCAGCTGGC |
| | TGCTGCTTAGGCGATCTTTGACCAGAGCTTTCCCTGAGAGTTATTTTTTCTTTCCCCCTG |
| | CTTCAATAGTGAGACTTTTCTTCAGTGTTGCAAGAACTGGCTTCTGTTCAACTTCTGGA |
| | AAAGCATTTAAAATTGAAAACGTTGACCTTCCAACCAAATCTTTGACCTGTTCTTGTTA |
| | AGCTTGAGATTTTGGGAAACGGAAGAGCTAGGAAACAAATCAGTCAACACAGCCAAA |
| | TGTGATATTGTGTCTCTTTTACACAATCTTACATTATCGTGAGTTGAAGAGAAACAGAA |
| | AAATGATAGTACATATAGTTAATTAGCAAATTAAAATACAAAAAAATGGTAAATATGT |
| | TGAAAAAGTGAAAAGGTTAGGGCAGGTGAATGAGTATCAGGAATGTGAAGGGTGAGG |
| | AAGGGAAAGAAATGCGTTTTCATTTTGAATGGAGAGGCCAGGTTCATTAAGCCTTTTT |
| | ATCTTTTCTTGAAAGCTCAATAATTCTTTATACTTTTGACTATTTTTTTTAACATTGCAG |
| | ACATGGGTAGGTGATTTTTAAAATAAATTCAAAATGTTTATACCTGTGTTGTTTTTATT |
| | AGTAATTCCATATGAATGGAAAATGTTAGTTTTAATGCTTATTCTTTTTATAGATCAAC |
| | TCCTACTTAAAAGAACTGCATTGAGCAGCTGCATTTTTGTTGTGGTGATATTTTTTTTTT |
| | AAAAGACATTCCCATTTCTGAAGAGTTAGAGAAACAGAAGGTTTCTCTTCAAATAGAT |
| | TTATCACCCACTGTTTCTGCAAATATCTGAGGTAGGCACTCCATAATATACAGAGAAG |
| | AGGACAGCGAAGCTAACAATTATCAGGCGTGTTTCATGTGCAAACACTGTGTTGGGCT |
| | CTGGGAATGTATATCATTTACTTTCTCAACAACTCTGTTAGTTGGGCATTATTGTTTTC |
| | ATTTTAGATGAGAAAGCTGCAACCCAGAGAATCAATTGCCTCTCTCAGCAAGCTAATT |
| | ATTTAATAGTTGGAGAAGTTAAGCCAAATTGATGCAAAATTTAAAAGAACAATAAAA |
| | TAGACAAGTTATTTACAATTCATAAAACACATCTAAATGATGACTTCAATCTATTTTCG |
| | CAGCAGTATGAAATGGATATTGCTATTCCCATTGTTACAGGACCCAACACTCACCCAA |
| | AGTTAACCTTTGGGTCGGGGGTTTCTGTACTATATTCCCTTCTGTGGTCGCCAGAGAAA |
| | TGTTACAGGAAAGGAGTCCCGATCCAGACCCCCGGAGAGGGTTCTTGGATCTGACGCC |
| | AGAAAAAATTCGGGGCGAATCCACAGGGTAAAGTGAAAGCAAGTTTATTAGGAAAGT |
| | AAAGGAGTAAAAGAATGCCGAAACCATAGACAGAGCAGCCCTCAGGGCTGCTAGTTG |
| | CCCATTTTTATGGCTATTTCTTGATGATATGCTAAACAAGGGGTGGATTATTCATGCCT |
| | CCCCTTTTTAGACCATATAGTGTAACTTCCTGACATTGCCATGGCATCATGACGCTGGC |
| | GGGAGTGTAGCAGTGAGAACCACCAGAGGTCACTCCTCGCCATCTTGGTTTTGGTGGG |
| | TTTTGGCTGGCTTCTTTACTGCAGTCTGTTTTGTCAGCAACGCCTTTGTGACCTGTATTT |
| | TGTGCTGATCTCCTATCCCATCCTGTGACTTAGAATGCCTTAACCATCTGGGAATACAG |
| | CCCAGTAGGTCTCAGTTATTTTACCCAGCTCCTATCCAAGATGGAGGTTCTCTGGTTCA |
| | CCTGCCTCTGACATCATTATACAGATTTAAAATGCTTCAAAATTAAGTGTCAAACTCAT |
| | GGTTTGACAACTAATTGATAGGAGAGGTAAGGTTTGAATTCATTATCTATTGATTATA |
| | AATTCTGATTTTTTTCAATGATAATGGCTGTACATGCTTAATTTTCAAGTGTGCCACCA |
| | GATTTGAGAAAAAATATTGGGTCTGACAAGGCCCAGGGTATTACGATTAATGCCAGTG |
| | AGAGAGACATCAAGTTAAGGAATCAGAGAATGTTGTTAGTGGTAGAGATCACAGACT |
| | GCGAGATTATCTAGACAGTGGTCAAGAGAGCTGAAGTGGGTTTGAAGATGGAGCAGA |
| | CTTGGGTAAGAGGACAGAATAACTGAGGTTATGAGGACAAGGTAGAAATAGGAAACA |
| | GAAATAATTCAAGAAAAGGCCTTGTTTTAGTAGGCATAGAGGCTTATCCCATACAAAC |
| | ATCTTTATATTATCCCTTTCCTCTTCTAAATTTGTAAATTTTTGCAAGTGCTGATCTGTT |
| | GAAGTGTTTTTGCAAATGCAAGCTTTCTTCCTACCCCCTCCTCTCCTCCTCCTCCCCTTC |
| | TCTTCCTCCTCCTCCCCTTCTCTTCCTCCTCCTCCCTTTCCTCCTCCTCCACTTCTCTTTC |
| | TCCTCCTCCTCTCCTCCTCTCCTCCTTTCCTCCTCCTCTTCCTCCTCCTCCCCTTCTCTTC |
| | CTCCTCCTCCTCCTTCTGATCAGGTTATTTGAGACAGGGAAGGGTAAGAGAAAAGGGA |
| | ATAACTGCGCTGTGACAGCTCCTGACAGCAGCTATGAAGTCTCACTTTCAGGCCTTTTT |
| | CTCTTCTATCTGCCCCACTGGAGCCTCTGGAAATTACAAAATAAGACATTTTAGGAAA |
| | ATGGTGCACAACTACTTACTTTAAAAATATTATAGAGTGCTACACTTCTGTGGATAGA |
| | GATTCAAAAATGTATTATTAAGGTTTTCTCTTGAGGAAAATGTTTCCTATATAACTGAG |
| | GGTTCAAAGCCTATTATGAGAGAGTCCACAATTCATGGAAATTGTTTTGTTTTATGGA |
| | ACCCCTTTTCCCTCAAGCAATGACTTGATTTAACACATTTCATAAAGCTGGATTTAGTG |
| | ACTGGTTTTTAAGAATTGTTTGGAAAAGTTGAGTGCTATTTTTTATGTAAATTGTTTCA |
| | TATATTATTCCACGGATTTTTTTTTAAGTCTGCTGTAAATCAGACACTGAAAATTTGTG |
| | AATGGTTGTCCTTAATATCAAGTGATTCACAATCTAGGGAGAGAAACAGTTACAGAAA |
| | CAATTGCGTGTATAGTAACATGTATTTCAATAGATGATATGCAAAGTATAGTGGGATC |
| | AAATATTTTGCTAGAAGTATTGTAGAAAAAGTTTGTATTTGATTTCAGTACTGAAGGA |
| | TGAATGGAGTTCCACTGGATAGAAGGTAAAGTATATTTAGTAGGAGGGAACAGCAGA |
| | CATACAGTCATGGAAGCATGGAAGATGACCTCTCATTCTGAGATGTGTTTAAGTACAA |
| | CTGGGTTTGGGTGGGTGACACGCATGAGACTGTGAATGTTGGCTGGGCCGGGGCATGG |
| | AGGAACTGTTTTGTATTCCAGGGAGATGCAGTGAGTGAATTGTAAGCATTAGAATGAG |
| | AAGAACAAATTTGCACTTAGAAAGATAATTCAGGAGGAAATGCAAATGATGGGCTAG |
| | GAATTGTGAAAAAGAGGCACAACTTGGTAGGCTAATGGAATTGTCAAGAATGGTGAG |
| | GTAGGGATTCGAATTTAGGCTGCTATGCTCCTCATCTTCCTTCCTCTCTCTCTGTGTCCC |
| | TCCCAACATCACCCTCTGCATAGATTCATGAGCTGTTAATTTTGTCCGTGATGCTCCTA |
| | TTGCTCCTCTTTTTTTCTGATTTTAGTTGAAGTGTCACCTCCCAGAGAAGCCTTCCTGAT |
| | TGGATCAAATGCCCGGCCCGGGCACCAAGAATTAAAGTTGAAACTTTAATTCTGTCCG |
| | TGTGATGTTGAGTAATACCCATTTCTTCCACTGGATTGTAAACTCCCTGGGGGCAGGG |
| | ATGGCGTCTGCTTTTTTTTTTTTTTTTTTGCTGTTCTTAGCACAACACCTGACGTAGATT |
| | AAGTCCTTAGTAAATTTGTTAAAATGAAATGAATGAGAGATGGAATCTAAATTGAAAA |
| | AAAGTTAAAAATTTAAACCATTCGAACTTGATTATTAGTTGAACTCCAAGATTGAGAA |
| | GGCAGAAATCAGGAATGTTTATAAAGTTTGTAGCTTGGGCAACTCAGAGGATAGTTAT |
| | ATATCCACTCAGAGAGAAAATAAGTATGCATGAGAAAGATAAAGCATTATATTTGAG |
| | ACATGTTGAATCTGAAGTATCACTAAGGCATCCAAATGGAAAGGTCCAAGGGGAAAG |
| | TGAAGATTTAATTGCTGATTGGGGAATTCATTAGTAACTTCATACATTCAACTGTTTTT |
| | CTATTTTTGGCTTTATCTTCCTTGACATCAGGTTGGTTTACACGTGATATTGTGAAGCT |
| | ACTGTTCTTCACTGGCAAGCTCCTGATATTCAGTGCGTTCCAAGAATATATTATTAATA |
| | AATTGAAATATTCTAACATAATTCAGTGAAGTCATGGAGAAACACAGATGATGATTCA |
| | AGTATAAAATTATCAATTTAAAATTAAAATATTAGATGAATAGTAAACTGAAGCAACA |
| | TTAAGTCATGATAATGCTTTTTTTTTTTTTACTCTTAGTGCCTATGAGGCAATACAAAA |
| | GGCTTGCAATTTTACATCTCCTATATTGCTTTAAGCACAAAACATAGTTAAAATATGAG |
| | TGGATATGTGACAATGATCAATTAGTGCTTAAGATTCTGTCTATACAAACACACACAC |
| | ACGCAAGTTACAGAAAATTATCATTTGAGGCCGGGCGTGGTACCTCGGCCTCTAATGC |
| | CGAGGTATTTAGAGGTATTAGAGGTATTAGAGGTAATTAGAGGTAGCTCACACCTCTA |
| | ATGCCAGCACTTCAGGAGGCTGAGGTAGGTGGATCACCTGAGGTCAGGAGTTCGAGA |
| | CCAGCCTGACCAACACAGAGAAACCCTGTCTCTACTAAAAATACAAAATTAGCCAGGT |
| | GTGGTGGCACATGCCTGTAATCCCAGCTACTCAGAAGGCTGGGAGAATCATGTGAACC |
| | TGGGAGGCCGAGTTTGCAATGAGCCAAGATTGTGCCATTGCACTCCAGCCTGGGCAAT |
| | AAGAGTAGAACTCTGTCTCAAAAAAAAAGAAAAAGAAAGAAAAGAAAATTATCATTT |
| | GAAGTATTTGTCATCATTCCATCAGCTGTGGTTTTTTCTATTCTATCAACTCCTTTCCAA |
| | CACCCGCTTCCAAAAAATACCCAGCACCCCTTTCTTACTTTCTCAGGAACACTATAGTG |
| | CTATTATCTTTCTGAATTCTCCAAAATTAAAAACTATTGGTCACGACTTCAATTTTTCTT |
| | CACAATGTATCTTAGATGCTGCATTTACATAACTGCATTTGAGACCAAACCATCCTCAC |
| | CCTATGTCTTTTCCCAGTAGATCACTGAGACCTTTGCTGCCACCCTCCCTTTACAGGAG |
| | CTCGCTTCACACACAGACAGGAACATGATTTTCCTCAAGTACAAGGTCTGTCAGGCAG |
| | ACCCCTCCCAAGATTGAGAACTCTACTTATAAGAATTTCTAGTAGGCCAATATGTACTT |
| | CTCGATCTAGTTTTCTCAAAATTCAAAGTTCTCGACTTTTATTACTTTTTTAAGTAAGCA |
| | ACCAAAGCAAACTGCTTATCGTTGGTCAACCTTATAAACTAGCTTCCCTCCTCTAAATT |
| | CATGTGTTTTAATTTCAGTGAAAATGTTAATTCAATACCATGCTTTCCCCATTTTCTTCA |
| | TAAGTCTTTGTCAAATATTAGGTCAATATTGAGATCTTTAAGTGTTTTTTTTTTCTAGTT |
| | AACATTTTTCTGCCACTGATTATTTATTCAAAGCTAGTTCAAGAAAAATTAAACAGCTA |
| | CATTTGTCATAACTTACGTTTACCAAATGATAAGTTTTGCAGAAATGAAAGTGAGGCA |
| | TTGCCGTCCAAGATCCTCCTTCTTTAAAAACATATGGTGCAATAAATAAGATGACAGG |
| | AAAAAAACCACACAAAGAGATAAGGGAAGATGCAGGACTTACATCTCATCTGGAGGA |
| | GGCTTCTTGAATGAGATGAGTGAGAAGCACTATGAAGAATCTGTGGGTGCAGGTGAT |
| | ATGGATGGGATAATCGTAATGGGAATGCTCTGTGTGTAATGTTTTTCATGGGAGACAC |
| | ATATAATCTGCAGTAATTGCGTTATTTATGGTAGTTCATATTAGATTTAGCTTTGTTGA |
| | AATAATGAATAATAAACCAGTTTCAAGTTTTAATGGATTCAGTTCCAGTACAGAAAAT |
| | AGTATAACAGATATTACCAATTCTCTAAACTTTCAAATCACTTTTTCTGATTAAATGGT |
| | ATTTAGACATTATACTTCAAAAGGTGGCCTTTCAAACTGTAAAAATGTTTCCCATATGG |
| | TTAGTATTTAAATTACACTCTAATTGTGAAGTATTGAATTTTATTGTGTTAAATATTGA |
| | TCAATAGCAAATTATTTTTGTCCTTAAATTCTTATTTTGGTAATTTTATTTTGTAAACAT |
| | TTTGTGATCATTATAATATTTTGTTTTTAGAAATGTTTTATGTGAAGTTTATTATGATGA |
| | GTAAATTCTCTTATGCTGTAAAACGGCATTTTATAAGATTATGTATTTTACACATTATT |
| | ACATCATAATCCCCAGAAATGTATATTCAGAAATGTGTCTCCATAGGCTCGGAAGGGT |
| | TTTAATGTATATTATTGCCTTGCTTAGTTAAAAGAAAAAACAATAACAAACATAAAAA |
| | AAACTATTCTTAACCTCTTGTGTAATTCCTTTCAATTGTAGTAGAGGGAGAGATGTGTG |
| | AAAGGCATTTGAGAACCAAACAATAATCAATTAATATGATAATTACTATCAAATTGTG |
| | TTTCTTATATTAAACTACGCATTTTCAAAACTCTTGTGGTTAGATATCTGTGTTACTGG |
| | AAGATATTAAAAATAATATTGCCATTTTTGCTCCTCTCTTTGTGAGGCAAATCTCTAGC |
| | TGAGCACTAATCTCCAGCCTAAGACCAGATCTATGTTGAATGAGAAAGAAAGTCTTAA |
| | AAGGCTGAATAGGAGATAAGTTGAAATAGCTGAAATAGTTGAAATTCGATAATAGCT |
| | ATCAAATTTCCTAAAGCGAATTGCCTTGGAAAAGTCGAGTAACTTTGTCTGGAACAAG |
| | GACAATTATTCATTCTGCATCCCTAGGCAATTTCACCATTGTGCAAACATCAGAGCAT |
| | ACTTACACAACTTCAGTGGCTGTAATTTAAACCTCATTATTGAAACATCAATTTTATAT |
| | CTTATTTTCTCTTCTGAAGAACAGTTCTTGAAAGAAACTTTTCTCTTTAAAGCACATTTT |
| | CTGCTAAATGTTAGATTTCTGCTACTACATGAGAGAATAGACCACTGTTTTTCAATGTG |
| | TAGGTTGTGACTAAGTTTTAATTGGGCTGTGGAGACTAAAAAGGAGACATATTCATGG |
| | TGTCAAGTCATTACTTATTAGGTAAACTTCTCTCTGACTACATAGAACATCTAAATCTG |
| | CCTTAGTTCATTCAAGGCTGTTATAACAAAATACCATAGACTGAGTGGCTTGTAAACA |
| | ACAGAGACTTATTTCTCCCAGTTCAGGGGGCCGGAAGTCCAAGACCAAGATGCCTAAA |
| | GATTTCGGTATCTGATGAGGACTTGCTTCCTGGCTTGTAGATGATGCCTCCTCACTGGG |
| | TTCTCACGTGGGGAAAATAGGGCAAGCTAGCCCTTTGGGATCTCTTTTATAAGGATAC |
| | TAATTCCATTCATGAGTGCTCTGCCCTCATGGCATAATCACCTCCCCAAAGTCTTCCTA |
| | ATAGCATCACCTCAGCAGTTAGGTTTCAACATAGGAATTTGGGGGCAACACAGACATT |
| | CAGACCATAGCCGTCTCCTACTGAAGTTTTTGGGAAACAGAGAAAGGGTGACTTGAAA |
| | TAGGAAGTCATGGCCGGGCGCAGTGGCTCACGTCTGTAATCCCAGCACTTTGGGAGGC |
| | GAGGTGGGCGGTTCACGAGGTCAGGAGATCGAGACCATCCTGGCTAACACGATGAAA |
| | GCCCGTCTCTATTAAAAATACAAAGAATTAGCCAGGCATGGTGGCGGGCACCTGTAGT |
| | CCCAGCTACTCGGGAGGCTGAGGCAGGAGAATGGCGTGAGCGCGGGAGGCGGAGCTT |
| | GCAGTGAGCTGAGATCGCGCCACTGCACTCCAGCCTGGGCGACAGAGCGAGACTCCG |
| | TCTCAAAAAATAAATAAATAAATAAATAAATGAATAAATAAACAAACAAACAAACAA |
| | ACAAACAATAAAAAAAGACAAAAGAAATAGGAAGTCATACATTCTGTATTTCTTTAG |
| | CCTTTATCAGCTGCAGTTGGGCAGCATGTTTCCACGGTGTCCTTCCATGATGTTTTCTA |
| | AGTTATTTGCCTTGATATTATTAAGAACAATAATGCTAACACCACTACTGATACAAAT |
| | AGTCAAGATTTATTTATTTTGCAGGTGTTATGCACTGAGTATAGCGCAAAGTGCTTCCC |
| | ATGCATTGTGTCATTTACTCTTTACAATATACGTATAACAAAGGAAAGATGCAAAGTT |
| | TAAAAATCTGACTTTGACGTTCATCAACATCCTCCCTTGTTTTTGATGTGGAGGAAAGA |
| | TGAGAGCTTTGTATGCTCTGATTGACCCTAGTGCCACTCAGAAACCTGTCAGGGTGCC |
| | TTGTGAATAATAATTACTTAAGAAGGATTTGTTGGAAAAATAAAAACTCTTTTAAAAT |
| | AGGTTGAATGTGGCTACTACTGTACTACTATAGAGACTGTACGCTTGGGGCTTTAGCT |
| | GCCCCGGAGGCTTTGTTATTCCTTTCTTTCATGCTGTCCTCATTCACTACCATTCTTCCT |
| | GTTAGGATGCTTTTCCATACTTCCAGCAGAACAGAGTCTGCCATAGTGACTAACGAAT |
| | CATGTTTTTCCTGGGGCACAGGCATGGATACCTTTCTTGTATAAGATTAATAAAAGAA |
| | TCCTCAGAGTGGACCAAAATGCTCTGCTCCTATCAAAATATCTGAAAATTGACATAAA |
| | AATGTGTTAGAAAAGCTTTATAGTTATAAAGGAATTATAGTTTTTAACAATATATTTTC |
| | ATCTTTAAATACCAAGAATTTCTTAACAGATTTTCTTATGTGACCACACCCTTTAATAG |
| | TATGTATTTATACAAACTCAAACTTATTTAGCTACTGCTATGTTATTTCAAGTTTTAAA |
| | TACATTCATGTGTTGCTTAATAACAGGGATATAGTCTCAGGGATACATTCTGCATCCCT |
| | GGGCAATTTCATCATTGTGCAAACATCAGAGCATACTTACACAAACCTAGGTGGTGTA |
| | GCCTTCTACACAACTAGGCTCTGCAGTACAGACCATTGCTCCTAGGCTACCAACTTGT |
| | ACAGCATGTTACTGTATTGAATATTGTAGGCAATTGTATAATAATGCAATGGTAAGTA |
| | TTTGTGTATCTAAACATAGAAAAGGTATAGTAAAAACTTGGTATTATAAATAATCCCA |
| | TTGAACCACCATCAGATATGCAGTTTCTCATTGACCTAAATATTATGCAGTGCATGACT |
| | GTATTAGAAATTTTTCAGCTAATCTCCATACAATCACCAACTAGTCACAGTAGAAGTT |
| | CCCTACTGTGTCAAGTTCATAAAATGTATACATTGAGAAGCATCTTTATAAAGTATTAC |
| | TTTAACTCTTAGTAAAATATTTGGTAGATAAAAAACTACACTAATATTTTCTTAATCTA |
| | AAACATGCAAGCTTAGATTTCTGTTTCATATTTTTCACAGACATTTACTGCTTTATTGT |
| | ATCTTCCCTACAGATCCAGCAGGTGTACTTAATTTGCTTTTGCCTAAAGGCATTTCTCC |
| | TTATTTAACACAATGTTATTTAGTTTCTTAAATATTGACTGGAATATTCTACATGTAAT |
| | ATGTGATGTGAATTGATGTTTTCAGGAAGCTTTACTAACTATATGCACTTGCATGGAA |
| | GTCTTCCATTTTATTTGTAAAAGATGTTCTATCTCTCTTTCTCTCTTTGGTCCAATATAT |
| | TTTACCAAAAGCTTGACTTTGGATCCAGAGTGAAACAGGTGGGGTTGAAATTCTGGTC |
| | CCATCACTTTCTGTCTCTGATTCCTGAACAAGCTGTTTGCACTTTCCGAGCCTTAGTTTC |
| | CAATATCTAATTTTTAGGATTCTTGTGAGGGTAAAAGGAAGTGATATATACTGTCTTG |
| | GTACACTGTCTGCACTTAAATCTGTTCAATACGTATTAGCATTTTTTTTTTTACTTTCAT |
| | AAGTGTCAGAAATAGTATTTTCTGTAGTAGTAGGTCATTATTTAATGTTAGATTTTTTA |
| | AAACCAAAGATTGTTTAGAATGTTCTTATAAGGTTAACTGTTTCATGTATTTGGTAGAC |
| | TTGCAAAACAAATGATTAAACTTGCTTTCTCTTCAGCCATTTCTCAGAGCAAATTAGGT |
| | TAAACTGTAAGGGTGATATTATATTTTAACAGCACATAGAGCTTTTCTGAAATGTCAT |
| | GAATTTCATATATATATATCATATATATAAATCAATGGGCAAATATAAAGTAACTTTA |
| | AGAATAATGTCATGCAATCAATGTAAAAAGTATAATTGATTTAGTGTTTCTGGAAGAG |
| | TGGGTTTCTAGCTTTGTGTTCTCAGTTTCTTTATTATTTCTACTCAAGTAACTAAAATGA |
| | TAAATTTTTGCTTTTTAGTCACTCAAGACTGCTTGCAACTGATTGCAGACAGTGAAACA |
| | CCAACTATACAAAAAGGTAATAAAATATAGCAAAGACTTGGAACCAAGCCAAATGTC |
| | CATCAATGATAGACTGGATTAAGAAAATGTGGCACATATACGCTATGGAATACTATGC |
| | AGCCATAAAAAATGATGAGTTCATGTCCTTTGTAGGGACATGGATGAAGCTGGAAACC |
| | ATCATTCTCAGCAAGCTATCGCATGTTCTCACTCATAGGTGGGAACTGAACAATGAGA |
| | ACACATGGATACAGGAAGGGGAACATCACACACCGGGGCCTGTTGTGGGGTGGGGGG |
| | AGGGGGGAGGGATAGCATTAGGAGATATACCTACTGTTAAATGACGAGTTAATGGGT |
| | GCAGCACACCAACATGGCACATGTATACATATGAAACTAACCTGCATGTTGTGCACAT |
| | GTACCCTAAAACTTAAAGTATAATAAAAACAAATAAAATATCCCCAGTACTTTTTCCA |
| | CTTAGAATTCAGTCCACTCCAAATATTTGTAAGTTTCAAGTCTAAAGACTGTATTTGAA |
| | TTGCATGTAAAATCAATCAGCCATCTCAGAACTTCTTATAATCAGGTAAAAAATAATA |
| | AACCTACATTTATTCAAAATGACTTGGGAATAAATAATAATTAAGACCAAAGTAATAA |
| | TCTAATAAGGAGAATGACCTTAGAGGCACGAATACTTAGAAATGTTAAATAATTAAAC |
| | TACAATAAAATAAAAACCAATAATAAATCAAATTCCATTTTTATTATTTAGACAGCAT |
| | TTTATCTTTAAGAATGACTCAAGACCATCTTCACAAATATGAATTCTCCTGTTATATTC |
| | AATTGTAAAAATCATAGAAAGAGGAAAATAAAACTGAACAGATTATTAAAAATCTGT |
| | ATTTTTAAAATAAATTTCCAACATCAAATTATTGCTTGATAATTTACCGTTTTCTCACA |
| | GAGGTAGAATGTTTTACATTTTTCAGTTATTTGAAGCCTTTTTTAGTTTTAATTTTAGAC |
| | AAGTTAAGTGTTTTCCATAATAATGGCCTGAAACCAGGTTTACAAAGAAACAACTTTT |
| | AATGTCATTTCCCAATATGTGTCACCTAGAATGTTAATCCAGAGAAATGGTCCTTGAA |
| | ATAAGAGTTTCAGTGTCGTATAAGTTTGGAAACACAGCATCTTACATATTTTTCTTTGA |
| | GTCACAAGGCACATTAGCAAGTTATTGAATCTAAGTAGTCCTACTTTAAAAAATGTTT |
| | GTTTAACTTTGTTTCATCCAGTTTTTCACAAATTTTTGTGACTATGCTGCTTTATCATGT |
| | ATCGACCTCTCTCTGAAAGCAAATGGGAATTGATGCTCTATAATAGCATTTGTTTATAT |
| | AAGATAACTAGTGAACAAAAATGTACTAAAAAAGAACCATTCGTTCACTCACACATTC |
| | ATTGATTCATTTAGTTTACATTTATTAATTAAGAATATACAATATTTTAATTACTGTAT |
| | GTTTACTGGGTGTATCAGTGACTAAAATATGCACAAATGCTCCTTGGCTTACAATGGA |
| | GTTATGTCCTGATAAACCCATCCTAAGCTGAAAATAACGGGCCTTGAAAAAAGCAGTT |
| | AGGACATCTAACTTACCAAATATCATAGCTTAGTCTAGCCTACCATAAATGTGCTCAG |
| | AACACTTACATTAGTCTACAATTGGGCAAAATCATCTAACATGAAGCCCATTTTATAG |
| | TAAAGTATTGAGTAGCTCATGTTGTGTTTTGAATACTGGAATGAAGTATGATTTCTCCT |
| | GAATGTATGTGGCTTTCACACCTTTGTAAAGTTGAAGATTCTAAGCCAAACCATCGCA |
| | AGTCCATATGCATGGTTTTCTACTTTGAAAGAGCTTACCTCTTGATGAGGAGAAAACA |
| | AACGAAAGGCCCAACAACCCACAAATGAACAAGTAAAATGTAGATGAGACGAAATGC |
| | TGTGGAGGGCAGCAAAGCAGGCGGGGGCAGGAGAAAGTTTTGTGTTGTGAACGAACT |
| | ACGAGTTCAGGAGTATCTGGAGGTTAGGCTAGGCGATGAGGAATGGGCAAAACAACA |
| | CTAGCACAGATGATGAATGTGAGTTGGCACTGGTAATGATTCCCTGGCATGCTCTTTTC |
| | ATCTGTGTTTTTTTCTGCGTGACTTTAATTGTCCTAAATGCAACACAAGTACTAAACTA |
| | CTTGTAAATCTATTATTTATTGTTCAACATTTTCCTAAATCATGTTTGCAACTTTTAATA |
| | AGCAGCACACTGTTTCCTAAGAAGTAAAAGAAAGCCAGAAGATCTTAAGGGCCAAAG |
| | TTACAGTTTTTAAAAATTCTTTTGAAAAGGTTTACTTTACACATCTCTCCCCCACCCCC |
| | CAAAATGCTATAAATTGCACCAAATTATGCAGCTGCTATACTCCAATGTGCGTGTGAA |
| | CTGGAGAGTATGCTGTGATCTTCCTGGAGTGGTGACTGCTTGCCACAGATCCAGGGGT |
| | GCTGAACTGGGGTGATTACCTGGGATGCTTGCTTGCAGTGCTCCAACCTTTAGGATGG |
| | ACAGGTTGCCCTTGTGATGTCAGCAAACCCTTGACCAGTATATAAATGTGAGTGTGCA |
| | TGGGCATTTAGCAGGCAGATTGCATTTAGCAGGTAGGTTGCATTTTGTTAAAAAAGGC |
| | TGTACTCACTAAAGCATTTTACTTGCACAGGTCCATAAGAAGACTTCTGGGAAAAGAT |
| | AATAATATATTATTAATATTATTAGCATATTATATGTAATTATATATTATATAATATAT |
| | AATGTATTATAATAATTATATATAATAATATATTATTATCTTCTGGGGAAAGAATTTAT |
| | ATATATAAATATTATTAGCATATTGTATATATTATCTTATTGTATTATACATAACATCA |
| | GGTCTTGAGATGAAATCATGTTATGTATTCCAGAGACATGGAGATTCAAAGGAGCGGG |
| | GCTACGTTCATCTGATATTTTGAGTTCCTATGCTTGCTCTGCCCCATACTTTTGTCCAGA |
| | CAGTGAGGGCAGAGGTAAACTTCACAAAAGAGAAAAAGACAAGAGGGGTTTATCAG |
| | AGTGTAGTCTCATGTCTTTTCTGTTCATTTGTTTTTTTGAATTTTAATCTAGATTTTGAA |
| | TAGGTAATGCATTCACACGTTGCAAAAAATAAAAAAGCATGAAGAGTGTGGATTGCT |
| | CCTTTTCCCATCTGTTCCTGCCCACACTCACCTCACTTTCAGTTTCCTACATAGACTTCA |
| | GACTCCCTCTATGTTAACACAAACAAATGCAAATGTAGCTCTGCTTCCCTCCTTTCTTA |
| | TAGTAAACACTTAAAAACCATATTTTCAATACATATGATTTGAAAATGAGATTATAAC |
| | CTGCATTCTTTACTTAACAATGTATATTGCAGACATTTTGATGCCTGTGTACAGAGATC |
| | CTCATATGAAAAAAAAAGCAGCATAGTATTCCATTGTATGAATTGAATGTATTTAACC |
| | AGTCTCCTACCAATAGACTTCTTGGTGTCCATCTTTTGCAATGCAGGTGCTTCGAAAAA |
| | TAATTGTGTGCATCCGTAACTTCTCATATGTCAGCCTATGTGTGGGACAGATTTTGAAT |
| | TTTGACAGTTATTGCTAAGTGGTCCTATAGGGGTTGTAAAGTGTTATGCTATCACTTGC |
| | AGCAAAAGAGCATCAGGTGAAAAATAGCATCTCTTAATTTGCTTTCTTTCAAGCCATT |
| | CTTTTGTATGTTTTAAGAACCATTTGTATTTCTGTCTTTACATTCTTCCAATTTTTCATGT |
| | CTATTTTTATTTTTTTCTGTGAGCCATCTGTTCATATTTTTTTCCATTTTTTCGTTGGACT |
| | TGGTCATTTTTAAACTGATTTCTGTGAGCACCTTATATCTCAAGCAGATTAATTCTTTA |
| | TATTACTAATTGCAAGCATTTCCTCCCATACTGTCATTTGTCTTAATTTTGCATACTTTT |
| | TTTTTCTGTCAAATTTGTCAATTTTTTTAAAGGATTCTGGATTTGGGTTGTGATTAGAA |
| | AGACCTTTTACACTTAAACGTATTAAGGAAAGATCTCGTGTTCCTTCTGAAACCTTTAT |
| | AGTTATTTTCTACCTTTAAATACTTTATCTATTTGCAATTTATCCTGGTATAAGATGTAA |
| | GGTATGAAACCAACTTAATTTTCCCCTGTGAGGGCATCACAGTCTTTTTTTAATCGCCC |
| | ATCTTTTCCAGACTGCTTGAAGACATTAACTTTAACTTTTATTAAATTCCATGTTTAGTC |
| | TATGTTTTTTTCACTTTTTAAATACTTTTCTGTTATACCATATTGACTTAATGTCTATTTA |
| | TGCACCCCAATAAAATGGCATTTTGGTTACTAGGGCTTTGTATTATTTTCTGGTAAATT |
| | TAATTCACTCATTTTTTCTTTTTACTTCTAGAATTTTTCTTTTTTTTCTATTAAATATAGG |
| | AACTGTAGAATCAGCTTACTTAGTTAAAAAGAAAAAGAAAAATACGTGCTATTTTTGT |
| | TGAATCTCATATTTATAAATTAAGAAAAATACCCTCATGATAACAGATGTACAGATCA |
| | AATCCAAGAACATGATATATATTTATATTTTTTTAGTCTCCTTTTTTTTCTTTTAGTATG |
| | TATTGAAATTTTCTTCAAGAAGTTCTTGCATATTTCTTATTAATATCATTACAAAGTGTT |
| | TTACCTTTTCAAATGCTATGATAAATAGAAACATTTTCTTTCATTATATTTTCCAGCTG |
| | GTTGTTCTTTTTGTGTATAAAAGCCTTGAATTCCATGATATATCTTTTCAATATTAAAGT |
| | TGAAATGTTTTCTTTTATTTTTGGATTGCATTTCCCTAGCATGACTTTGCCCAAAATTTT |
| | ACTTTTAACTTCTCTGAAATAATTTTTCTGTGTTTCTCATGTATGCAAAACAGAGTTGC |
| | TCTTTGTTTTTTGATATCATCTTAAAATAATTTTCATTTATTGGGTGATATAAATCAATT |
| | TACATTTACTTATTTCAGAAATGTATTTGGCTTTTATTCATCATAATGTGCTATACTGTG |
| | TATTGTTTTTCATGTTAACAAAAGAGTTTCTATTTTATAGTTTTAAAGAGGTTGCTTACT |
| | ATGTAGGCTATTTGCTTTGCTTTTTCTGTTAGCGTAGACTTCTAAATGCTAAATACTAG |
| | GGGTAGCTACCTTTTCAGTTATATCTTTTTATTGAAATTAAATTCATATAACATAAAAT |
| | TAAATGCACAATTCATTGTAATGTAGTATTCACAATATTTTACAACCACCAACTCTCTC |
| | TAGCTCCAAAATATTTTCTTCAGCCCCAGAGAAAATCCTGTATCCATTAAGCAATCAG |
| | TTACCATTTCTCTGTCTCCCCATGCCCCTGTACCAGCAATCTGCTTTATGTCTCTGTGGA |
| | TTCACCTATTTTAGATGGTTTATATAAAGGCAGTCACACAATACGTGACTTTTAGTGCC |
| | TGGCTTCTTTCACTTAGCATGATGTTTTGAGGTTCACCCACATTCTAGCATGTATTCAT |
| | ACTTCATGCCTCTGTATGAATGTATATCATTCTATTATAAGTAAGCCATAATTTCATTA |
| | AGGGTATTTGGTTTGTTTCCACCTTTTGGCTATTGTGAGTAGTGATCCTGTGAACATTT |
| | GCATAAAGTACTTCCTGAGTGCCTCTTTTCAATTATTTGGAGTATACATCGATGAATCG |
| | ATGAGTAGAATTGCTGAGAAAATGTCAACCTGTTCTCTACAGTGCTGCAACATTTTGC |
| | CTTCCCCCCAGCAATGTACTAGTGTTCTAATTTTTCCACATCCTCACTGACACATCTTA |
| | TTTTCAATGTTTTTATTAGTATTGCCTTCCTACTGGGTGTGAAGCCATATCTCTGTTATT |
| | TATTTGCATTTTCCTTATGACTGATGATGTTGAGCATCTTTTCATGTCCTTTTTGGTGTC |
| | TTCTTTGGAAAAATGTCTATTCAAGTACCTTGTCCATGTGTTTTAATTGGGTTGTTTGTC |
| | TTTTTGTTGTTGAGTTGTAAAACTTCCTTATATATTATGGATACAAGACCCTTATCAAA |
| | TATCTGATTTGCAAGTATTTTCTCCTCTTTTGTGGATTATCTTTTTACTTTCTTGAGAAT |
| | GTCATTTGAGGCACAAGAGTGTTGTACATCAGATAAATGAAGTAAAATTTATCTTTTTT |
| | TTGTTTCTTGTGATTTTAGTGTTATCATTGAAGAACTCATTGCCAAATTCAACATCATG |
| | ATTCACCCATATGTTTTCTTCTAAGAGTCTTGTAGTTTTATGTCTTATATTTAGGTAATT |
| | AATCTATTTTAATTCATTTTTGTATATGGTGTGAAGCAGAGGTCCAAAATCATCTTTTA |
| | GCATGAGGATATCTAGTTGTCCCAACATCATTTGTTGAAGAAGATGATTTCTTCCACAT |
| | TGAACGGTCTTGGCACACTTGTTAAAAATCAGTTGTACGTAGATGTATGGGTTTATTTC |
| | TGTACTCTCAAATTTATTTTGTTTCTTTATACACCTCGCCTTATGCTGGACCACTATACT |
| | ATTTGGATTACTGTTATGTTATAGTAAATTTTGAAATTTAGGATAGTGAATCCTTCAAT |
| | CTTGTTATTTATTTTTAAGATTGTTTTGGCTATTTGATACTCTTTTGCAATAACAAATGA |
| | ATTGTAAGGCTGGCTTTTTCATTTTGGAGAACAAAAGGTCACTGAAATTTGTATATGG |
| | ATTAGACTGAATCTGTACATAATACTGAGTATTACTGCCATCTTAAACTAAATCTATCA |
| | ATCCATGAACACAAGATATCTTCTCTTTTATTTATATGTCCTATCATTTCTTATAGCAAT |
| | CTTTTGTAGTGTTCAGTGTTAAAGTGTTTTATGTCCTTGGGTAAATTTACTTCTAGGTGT |
| | TATGTTCTTTTGAATGCTGTTTAGTCAGTTCAGTTTGCTATAACATATTACACTAGACT |
| | GAGTGGATTAAACAATAAAAATGTATTTCTCACACTTCTGGAGGCTGGAAAATTCAAG |
| | ATTAAGGTGCCAACAGATTTGGTTTGTGAATGGTGGTCTTTTCATTGTGTTTTCACATG |
| | GCTGAGAACAGAGACAAAAGCAAGTTCTTGTGTCTTTTCTTATAAGGACATTAACCCA |
| | TCCTGAAGGCTGTGCTACCACAGCCTAGTTATTTCCCAAAGGCACCACCCCCTAATAC |
| | CATCACATTGGAAGTTAGAATTTCAACATATCAATTTTGGGTGACAGAAATATTCAAT |
| | CCATAACGGATGCTACTGTAAATGGGATTCTTTTTTAAAATTTCCTTTTTAGATTATTC |
| | ATTCCTGGTATATTGAAACACAACAGATTTTTGAGTGTTGATCTTGTACCCTGAAACTT |
| | AACTGAATTCATTGATTAGTTTTAGTAGATTTTTTTGTAAATTCATGGATTTTTTTTTTT |
| | TTTGAGATGGAGTTTTGCTCTGTTGCCCAAGCTGGAGTGTAATGGCATGATCTTAACTC |
| | ACAGCAACATCTGCCTCCTGGGTTCAAGTGATTCTCGCACCTCAGCCTCCCGAGTAGC |
| | TGGGATTACAGGCGCCTGCCACCACACCCAGCTAATTTTTGTATTTTCAGTAGAAACA |
| | GGGTTTCACCATGTTGGCCAGACTGATCTCGAACTTCTGACCTCAAGAGATCTTCCTGC |
| | CTCAGCCTCCCAAAGTGTTAGTATTACAGGCGTGAGCCACCATGCCTGGCTGGAAATT |
| | TTCTGTATATAGAATTATGATATCTGCAAATAGAGATCGTTTTACTACTCTCCTTGCAT |
| | TTGGATTTTTTTTTTAATTTCCAGCATATGTTGAATAGCAGTGGTAAATGCAGGCATAG |
| | TCTTGTTTCTGATGTTAGGATAATCTTGAGGATTATTACCTTTATATTCATGGTTGATTC |
| | TGGCCTGTAGTTTTTTGTGGTGTCTTTGTGTGGCATTGATATCAGTGTAACTCTAGACA |
| | TACAGAACAAGTTAGGAATGGTTCCCTTCTCTTTTACTGTTTTTTGGAAGAGTTTGAGA |
| | AGATTGATGTTAATTTTTCTTTAAATGTCTAGTAAAATTCACCAGTAAAACCATCTAGT |
| | TATGAATTTTGAGGGAAGACTTGGATTAATTAAAACTCTACACTGGTTATAGGTCTGTT |
| | TAGGTTTTTTATTTATTTTTGAGTCAGTTTTGGTAGTTTGTGTGTTTCTAGGAATTTGTC |
| | CCATTTGTCTACATTATCTAATTTGTTGGAGTACAATTATTATAAATAATAAAAAGGTA |
| | TAGACATTTTATTTCCAATGGGCCATTAATAATACCTCCATTTTCTTTAATGATTTTATT |
| | AATTGGAGTATTTTCTCTTTTATTGGTCAATGTAGCTCAATGATTGTCATTTTTGTTCAT |
| | ATTTTTCAAAAAACCAGTTTTCAGTGTTATTGTTTCGTCTTTTTCTGTTCTTTTTTTGAG |
| | ATGGAGTCTCCGTCACCCAGCCTGGAGTGCAATGGTGCGATCTCGGCTCACTGCAACC |
| | TTTGCCTCCCAGGTTCAGGCGATTCTCCTGCCTCAGCCTCCCAAGTAGCTGGGACTACA |
| | GGCATGTGCCACCAAGCCTAGCTATTTTTATTTATTTATTTATTTTTTGTATTTTTAGTA |
| | GAGACTGGATTTCACCATGTTGGTCAGACTGGTCTCCAACTCCTTACCTCAAATGATTC |
| | ACCCGCCTTGGCCTCTCAAAATGCTGGGATTGCATGTGAGCCACCGCACCCAGTCCTT |
| | TTTGTACTCTTTATTCCATCTACCTTCTTTATTATTACCTTCTTTATTATATTATCTGATC |
| | TTTATTATTTTCTTCCTTCTGTCAATTTTGGGCTTAGTTTCTTCTTCTATTTCTATTTCCT |
| | TAAGCTGAACTTAGGATATTATGTTGAGATCTTTTTTTAACATAGGCATTTACAGTTGT |
| | ACATTTCTTTCTGAGTGCTACTTTCACTGCTTCCTATGAATATTGCTATGCTGTGTTTTT |
| | ATTTTCAATCATCTCAAAGTATTTTCTATTTTCTTCTAGGATTTATTTATAGATCAATTT |
| | GTTGTTTAAGAATTTGTTCAATTTTCACTTATTTGTGAGTTTTCCAATTTTCCTTATGTT |
| | ATTGAAGGTAGCTAAAATGAATGCTTAATAGAGAGCCATTTCAGATTGAAAAACATA |
| | AACAGGTTTCAAGGTCAAAGGATGAAAAACATATCTTTTTAAAATACAAGATATTTGT |
| | GGAATTCAAATATTTTAAAATTTAATGAGAATTATTTTGTTGCATAACATGGTCTATCT |
| | TGGTCGATGTTCATGTGCATTTGAAAATATGTGTTCTGCTGATTTTAGGTGGAGTGTTT |
| | TCTATGTGTCTGTTAGGTTCAGTTAGTTTACAGTGTTGTTCAAGTTCACTATTGCCTTAT |
| | TGATCTCTTACCTAGATGGTCTAGCTATTATTTCAGGTGAGATACTGAAATCTTCAGCT |
| | GTTACTATAGAAATGTCTATTTCTCCTTTCGGTTCAGCCAATATCTGCTTATATCTTGA |
| | GGTTCTGTTGAGTGATGTTTATAATTATTAGGTATTCTTGATACATTGACTCTGTTATC |
| | AATATTTAATACTCTCTTTGTTTCATAAAAATTTTTGGCTTAACATGTATTTATTCTGAT |
| | ATCAGTATTAGCCATCCCTGTTATCTTTGGCTGCTATTTGCAAGGAGGCATATTTGTCA |
| | TCCTTTAATCTTGAAACCTTGAAACACAAAAACCTGTTTGTGTTTTTGAATCTGAAGTG |
| | AGTCTCTGGTAGGCAGAATATAATTAGATCATGAGTTTGGGCTTTTAAAAAAATTATT |
| | TTACCAATCTCTGTATTTTAATTGCCAAATTTAAATTTAGCTCATTTATATTTAAAGTG |
| | ATTACTGATAAGGAAAGAATTACTTATGTTGCTGTTTGTTCTTTTATAAGTTTTGTTCCT |
| | CAATTCCTTTATTACCTCTTTATTTTGTTTTTAATAGACTTTTATAAGTACATCATTTTG |
| | TTTTTCTCTTAATTTCTTTTTGTGCATTTTTAAAAGTCATTATCATAGTTGTTATTCTGGT |
| | GATCACAATAAACATCCTAAATTTATAACAATGTGGTTTGAATTGATAACAACTTAGC |
| | TTCTCTCTTATGTTGTTATTGTCACAAAATATACGCTTATACATTGTAAGCCCTTAACA |
| | TAAATTAATAATTATTATTTTATGTATTTGCCTTTTAAATAGTATAGGAAACAAAAGGA |
| | GAAGTTAAAAACTAAAAATACACTTACAATGGCTTTTATATTTGCCTATGTATGTACCT |
| | TTACTTGTATTCTTTATTTCTTCATATGTCTTTCAATTCTCACTTAGTGCTTTTTGTTTTA |
| | TTTCTATCTCCTTTAGCATTTCTCACAGGGTAAGTCTGCTACCAATGAACTCTCTCAGT |
| | TTTTCTTTATTTGAGAACATCTTAATTTTTCTTCACATTTTAAGGATATTTTCCCACATA |
| | TAGAATACATGGTAGACAGTGTTTTTCATTCAGTACTTTAAATATGTAATTCCCCTGGC |
| | TTCTGGCCTCCATGGTTTTTGATGACAAATCAGCTATTAATCTTATTCAGCATATTTTAT |
| | ATTTGATGAATTGCCTCTCTTTCTGCTTTCAAGATCTCCTCCTTGTCATTATCTTTTAAT |
| | AGCTTGATTTTAAGAAGTCTTGGTAGGAATCTTTTGTGTTTACCCTTCTTGGAGTTTAC |
| | TGAATTTTCTTAGATGTGTACATTCATGACTTTCTTCAGCGTTGGAAAGAGTTTGGCCA |
| | TTATTTCTCCAAATATTCTTTTTTCAAATTTTCTCTCTCTTCTCCTTCTGTAACTCTCATA |
| | ATGCCTATGTTCATCTGCTTAATGTATCCTGGGGGTCTCTTATAATCTGTTCACTTTTCT |
| | TTTTTCTTTTTTATTTTAACTTTATTTTTAGAGCCATTTTAGATTCACAGAAAAATTGAA |
| | AGGAAGGTACAGAGATATCTCATAGACCTCATATTCCCTACGGATGCATATCCTCCCC |
| | CATTATCAAAATCCCCCACCAGAGTGATACATTTGTTACAACTGATAAACCTACACTA |
| | ATATATCATTATCATCCAGAGTCCACAGTTTACATTAGGGTTCACCTTAATGTTTTACT |
| | TTCTATAGGTTAGCACAAATGCATAATGGCATGTATCCACCATTGTAGTATTAGAGTA |
| | TTTTCACTGTCCTAAAAATTCTCTGTGCTTCACTCCAACTACCTTTCCCTTTCCACTGCT |
| | TATCCCCTGGAAATGGCATTTTTTAAATGTCTCTGTAGTTTTAACTTTGTTCCAGAGTG |
| | CCATGTTGTTGGAATTGCACAGTATGTAACATTTTTATTTTCACTTCTTTTTTTTAGTAA |
| | TAGCAATTTAAGTTTCCTTCATGATATATCAGTGTAGGTTTATCAATTGTAATAAATGT |
| | ATCACTCTGGTGGGGGATTTTGATAATGGGAGAGTATCTGTAGGGAGTATGAGGTCTA |
| | TGAGATATCACTGTACCTTCCTCTCAGTTTTTCTGTGAATCTAAAATGGCTCTAAAAAT |
| | GAAGTTAAAAAAGAATAACGAAAAGTGAACAGATTATAAGAGACCCCCCAGGATACA |
| | TTAAACTGATGAACATATGCATTATGAGAGTCACAGAAGGAGAAGAGAGTTAAAATC |
| | TGAATAAATAATATTTGGAGAAATAATGGCCAAACTCTTTCCAATGTTGAAGAAAGTC |
| | ATAAATTTACATATCTAAAAAGTTCAGTAAACTTCAAGGAGGGTAAAGACAAAAAGA |
| | TTCCAAATCTTATTAAAATCAATTATAGTAAGTCTTGAAGTAGAGTAGTGTTAGTTCTC |
| | CCATTTTGTTCTTATCCTTTAATATTATGTTGGCATTTCTGTGTCTTTTGCTTCTCTATAT |
| | ATTCTTTAGACTCAGTTTGTTGATATTCAACTTGCTTTGATTTTGATTAAGATTGCATTG |
| | AGTCACCTGGGCACGGTAGCTCATGCCTGTAATCCCAGCACTTTGGGAGGCCAAGGCA |
| | GGATCACAAGGTCGGGAGTTTGAGACCAGCCTGGCCAATATGGTGAAACCCCTCTCTA |
| | CTAAAAATACAAAACTTAGCCCGGCGTAGTGGCACTTGCCTGTAGTCGCAGCTACTCG |
| | GGAGGCTGAGGCAGGAGAATCACTCGAACCCCAGAGGCAGAGGTTGCAGTAAGCCAA |
| | GATGGCACCCCTGTCTCCAGCCTGGGCGACAGAGCAAGACTTGGGAAAAACAAACAA |
| | ACAAACAAACAAAAAACAAACAAACAAACAAAACATTGCATTGAATCTACAGGAAG |
| | AACTGACATCTTGACAATATTGAGTCTTCCTATTCATAAACATGGAATAGCTCTCCATT |
| | TATTTAGTTCTTTAAATCTTTTACATATTTTGTTGGATTATTCCTCAGTATTTCATTTTTT |
| | GAGGTAATAATCTCAATGCCATTATGTTTTTAATTTCAAATTCCACTTGTTTATTACTG |
| | GTAGAAAAGTGTTAGATCACTGGTAGAAAAGTGATTCGCTATTGAGTATTAATCTTGT |
| | AATAATGACTTTATTCTTTTGGTCCGCTGAGTAGTTCATAATTTTCTCTTTCTTTGTATG |
| | CCTTGAAGTTTCTTCTTGAAAAGTGAACAGTTAGGCAGTTAGGATGTTACAATGTAGC |
| | AAATCTGGAAATCAAAATTACTCCCATCCTCAGGGTTTTCTGTTAATGGATGTGGACT |
| | GCAGTTGTTCACTTGTTTAGTAATTTTTCCAAAATACTTTTTTTTTTGTTTGCCTAGACT |
| | CTATTCCTTGTCCTGTGTGGACACAGGAGTCCTTCTTCTATTATCTCTTTTGTTGGCCTG |
| | GTAGAGATGATTTTATCGCTAATCCTCATCCTGACAGAGATTTCGTTAAACATTAAAA |
| | GTCAAAAAATAAAAATAAAAATCTCTCCTGGTATTTACAAACAGCCTCTGAACTGGGG |
| | TATTTCTTCAAACTTTCACTAGGCTGTCTGCAGTTAAACTTAGTCTTTACCTCCTGTTTG |
| | CAGAGAGCCCAGGTAGGTATGTGGATGTGCGTATGTGTGTGTGTGGATGTTGGAGGAA |
| | TAAAGACAATATATAAAAATAAGTAAACATGTTCTCTATCACTTTAATAAGATTATTG |
| | CCAGAAAGAGATGATAAAATGCCTAAATAAAATGTTAAGAGACAGTAAGAATAGAGA |
| | CCTACTAAGTATTTGTTTTATTTTCTAGGCTGACAATGCACCAGTGACATTTACTAAGA |
| | AGTGGGTCTTTAATTTTATACATATAAATTTAATGTAAAAAATCTAACAAATCAATCTA |
| | AATTATTATAAAATTTTAGTAAACAAATATCTCTAGCCAGAATGTAAACAAGTGGTTT |
| | TAGAAATTTGTATTTTACTTAAATTTAAGAGAATATACTACCTCTATGATGGAGGGCAT |
| | TTCATTACCAAGAATTTCAGTAACTTGGTATTAAAATGCTTTTAAAAAAACAATTGAA |
| | TACCTGTGATTAATTTTTACAGTACATTGCTATTTTGAATTATATTATGTTTAAATTTGA |
| | ATTCATTAATTTAATAAATATTTGCTGCATGTCTACTGTGGGTCAGTCATGATGTTAAG |
| | CCCTGGGAAAATGACAGGGAACCAAACATAAATGGCCCTTTAGCTCAGAAAATTTAG |
| | TATCTCTCAAGTATATTATTTTTTTAAAAGCTCTCTTCCCTCCAAGCATCAAATGAATA |
| | TCATTAAACTATGAAAGCAACTGCCTTGGTGAAGTCATCAGTTTGTGTTTTACAATAAT |
| | GACAGTAACTGTATTAACTCTTCCTCATTTTTCAGCACTCCAAAAAGTGCTTTGCATAC |
| | ATTATGTCATTTAAACAAAATTAAAAACTATTTTATTATAATCTTTTCACTGAAATTAT |
| | ATCGTAAAAGAGGCGATGATGATTTTGCATTTGTAATTTGTCATTGAGGTGATCTTGTG |
| | TTAACTATTACTATACAAGAGGGCTTTTCTTTTAAGGAAAATATTCCCTTCTTTGGGAA |
| | AAGCCAATTAGACCTGCCCATATCAAGGCACATTCACCCTTGGTGAGAGACAGTGACA |
| | GAGGTGGCTCCTGGGAGTGAGCAAGAAGCACAGGCCGACAAGAACATGTGCTGAAGA |
| | AGACCTGGGAAGTTTCTTGATCCACCCATAGATCCTCCATCCTTCCTCTGCACTCTCCC |
| | TGACATCTTTCCTCCCTCTGTCTATGGTAATAGGAACTCAAAGATCTGACTAGGGAAA |
| | TGTTATTTATTTCAACTTGAATTTACAATGTAATTTGCTTGGTTATCACCTAGGATTCTT |
| | ACTCCTTAGAAACAATCAGAAATATGTGGTTCTTATTTTATATTCTTCCCTCCATAATC |
| | TTATTTGGAATGGGAATATAAATTAGTGTAGCCATTATGAAAAATAGTACACAAGTTT |
| | CTTTAAAAATTAAAAATAAAATACCATATGATCCAGCAATCCCACTACCAAGTATATA |
| | TCCAAAGAAAATGAAATCAGTGTGTCAAAGAGATATCTCTCATCCAGGTACTAACCAT |
| | GCCCAACTCTGCTTAGCTCCTGAGGTCAGACAAGATAGGGTGCATTCGGAGTGGTGTG |
| | ACCATAGAAGAGATAACTGTACTTCAATGTTTATTGCAGAATTATTCACAATAGCCAA |
| | GATATTAAATCAACCTAAGTGTCCATCTGCAGATGAATGGATGAGGAAAATGTGCAAT |
| | ATATACACAGTGGAATACTATTCAGCATAAAAATAAGGAAACCCTGTTGTTTGCAGTA |
| | ACATGGATGAACTTGGGGGACATTATAACAAGTGAAATAAGCACAGAAAGACAAATA |
| | CTACATGATCTGACATATATGTGGAATCTAAAAAAAGTCAAATTCAAAGAAGTAGAG |
| | AGTAGAATGGTGTTGACCAGTGGCTGGGGTGGCGGGGGTGGAGGTGAAAAGACGTTG |
| | GTCAAAGATTACGAAATTTCAGTTAGACTGGAGGAATACGTTCAAGAGATCTCGTGTA |
| | CAACATGGTGACTACAGTTAATGAAAATGTATTTTTGAAAAATGCTAAATGAGTAGAT |
| | ATTGTGTTCTCACCACAAAAATGACAACTGTACGAGGTAACATATATTTTAATTTACTT |
| | GACTCAGTCATTGCACAATGCCTATATATTTCAAGACATCATGTTGTACATGACAAAT |
| | ATGTACAATTTTATCTGTCAATTAAAACATAAGATCAAAAAAGAATTTTATTTGGAAA |
| | AAGTTTAAATATGTTTGGTTTGCCTTGGAAACACTATGTGAAGAAGGCATGTACACTT |
| | GGCAGTTGTATTTACAGAATGTGAACACACTGTTAGCTACCACATCATCCTGTGCCCCT |
| | CATGAAAACCCACAGGAAAAAATATTCTTTAATCTCAATTCTAGAAATATTGTTATCTT |
| | CTTGGGCTTCTAGCCTATTGTCTGTTGTGGATTCTGAATTTTATTATCAATTTTGTTTTC |
| | ATTTTTGCTATGATCAGGATGAAATTTGGAGACAGGTATGGTTCACCATTACCAATTAT |
| | ATAGGGACATTTAAAGTATGATTTTAGACTAACTTCTATAGCTCTATTTTACTATTTTA |
| | TCCTTATATTACAGTCCCTCAATCTTCTTTTTATTCATTTTACATCATTTAATTTATACTC |
| | CCCAAATTTCCCAATCTCCAGCCTTAGGGCTCACCCTTTATTCAACACACCCATGAGAA |
| | ATCCAGGGAATGTAAAGTATGAATTCACAGGAAAGGAATGTCTCTGCTCTCTTAATGG |
| | GTTTTGATGTCTGAACGCACCTACAGATTCTGGCTCAGCAATGTAGTCCCCCCATGTGC |
| | CTACTCACTGATGGCTACTGTCTTCTCTCTGTCCCTTTCATCTCACCCTTGAGCACAGTG |
| | GTCTCCTTGTCACTACCCAAGTGACTTTTGATGGGTAATGGAGGAGAATTATCACTGA |
| | ACTGAACAGAAGCCATTGAAACTCAAATATCATAGATGGTTGTTACAAAGTTCCTTGA |
| | CCGTTCTTAGTAAAGGTTGTGTCTTAGTCACTTTCTTCTTTCTTCCGTAGGCTCCCTTCT |
| | GTTGCCACATTTGGGCCAAGGAATGGAGAGATTTCTTCGTCTGGAAACATTTTGCCAA |
| | ACTCTTCAGATACTCTTTCCTCTCTGGGAATCAAAGGAAAATCTCTACTAGTGTGTTAT |
| | TCAGAGAAATTTTGTCCACTCTTTGCTTCAAACTCTCTTATCCAGATCAGCCTTTATTAT |
| | GAAATAGCCGAAATATAGACCAATAAAGGGAAAACCCAGACATTCATATAAAGGGTT |
| | AGCTCTCAGGTTCTTGTATCTGATATATTAACCCAGGTTAACATATAAGTGATGGCAA |
| | AGAATCCAGTGCAGTAATGTGACTTGTATTCCTTTTTCCTTTTTTTTCTTTCTTTCTTTCT |
| | TTTTTTTTTAGGATGGAATTATCTTCTAAGTTTCAGAGGTAGCTTAACAACTAAATGGA |
| | GGTGAAAACTGCTTTTCTTGGTTTCTTTCCTTATAAATGATTCTCTTCATTGCAGGATCT |
| | TACACATTTGTTCCATGGCTTCTCAGCTTTAAAAGGGGAAGTGCCCTAGAAGAAAAAG |
| | AGAATAAAATATTGGTCAAAGAAACTGGTTACTTTTTTATATATGGTCAGGTAGGTTG |
| | AAACCCTAATTCTGGTTTTACTGTTCAAAGCCTCCCATTTTGTGTTCATTTCTGACACA |
| | GTTTTTTGGTTTGTTTCTTAGGTTTTATATACTGATAAGACCTACGCCATGGGACATCT |
| | AATTCAGAGGAAGAAGGTCCATGTCTTTGGGGATGAATTGAGTCTGGTGACTTTGTTT |
| | CGATGTATTCAAAATATGCCTGAAACACTACCCAATAATTCCTGCTATTCAGCTGGTA |
| | AATATTAGTTCTCACACCCTGCTAATTGTGAAATGAAGAGACTTTGACGAAAAATCTG |
| | AGCTGCAAAATGCAAAAATGAAAGGATGGTGCCCTAAAATACAAATAGACAGAAAGA |
| | CATTCCTCAATATATTGTGTTTTTTAAATCTTGAATAATAAGCTAGTCTGTTTTAATTGA |
| | GAAAGATGGAATTATTACTAATAGTTTTACCATTTTTATATACATTTAGAAAGGGTAGT |
| | AATTTCATAGCAACTTCTACCTTGTACATAATACATGGTACATAGTACTTCTACATAGT |
| | AACTTCTACATTGAGAAAAAATTCTAATGTATTTTCTTCAACACTTAATAACAACAATA |
| | ATAATATCTGACAAGGATAGAGTGATTACCACTTGCCAGGCACTCTTTTCTGCATGTTA |
| | TATATGCTTGCTTATTTAATCTTCGCATTTAAGGGAGTTAGGATCTAGGCACAGAGAA |
| | GTAACATAACTTGTCCCAGGTTACACAGCTAATTTTTAGACTTGGTTTAAACCAGCCA |
| | GTCTGGCTCCGCACCGGGAGCAACCAAAACTCTTCTTGCATGCTGTGGAAGAAATGCC |
| | CCTGGCCAGGCTTGAGGGTAAGGGAAAGTTGTACAATGGCTTCTTTCTAAATACAGGT |
| | GACATCTTGTGTCAGTGGACACCAGACAGGAGAAATGCCCCTGGCCAGGTTTGAGGGT |
| | AAGGGAAAGTTGTACAATGGCTTCTTTCTAAATAGCCACAAGTATTGACAATTCAGAT |
| | GACATCTTGTGTCAGTGGACACCAGACAGGTGACTTAGGAATGCGAAAGTGTAGGCG |
| | CAAACCTACCTTTGCACCCAGGAGCAGTGAGAGGCAGAGAGCACGGCAGATCTTGTA |
| | AACTTTAGGTCTGTTTCAGCTGTAATTGCCAACATTTCCTTCATTTTATTTTTCAAGGGT |
| | GATTTAAAATTAATCCAGGCTGGTTTTGCAGAAAAATATTTACCTTAAACACATTTATT |
| | TTCTCTGTTGATTTTGTAAAAATCCAGAACCCTGAGGAATGGCTTCAGTTCCAGATGGT |
| | TACCGTGAATGACCGTGTGACACACAGGAACGGAGGCTGGCTATGCATTTTGAAAAA |
| | CTGTATTTCAAAGGCATTTACAAACAAATGCCCAATAAGTCAGTTAGAAACTAAGGAA |
| | AATAGTTTCTAATAATGGAAATGATGTCATTCTGGATCTGCAGCCACAGATAGCATTG |
| | GTGGGGAGATGAGGAAGTTTATAAAATTAGAATTCCCTAACTTTCATAAAGAGATGTT |
| | TCCCTTCACCCAGTGCTGACTAGTAGTTTGGAGTTGCAAAATATTTATAAATGGTGAG |
| | ACTACTCCAATCTGTTATTTATTTATATTTATTAGTACTAAAAATTTTACAAGTAACTCT |
| | TTAAAACGTTTTTCTAATCATCTGCTCCTCTAACACTTACTTGCTGTTAGAGGACTCTG |
| | CTAGATTTCTTAAGGTGTCTTACTTTTTAAGAATAATTCACCGGACTTTAAAAATATTT |
| | TTCATTACTAGTAGCTTACTGTTGTATATGAGTACCATCCTCTAGTAAGTTTCAGGTTT |
| | GCAATTTTAATCTGTTTTAGGGCAAATTTATTTAAAGCCATTTAAAACCACACATTAGA |
| | GTATATATAAGTACATAAAATGTTTCCATAGCAACCTGAAAAGATGTAAATGGCCAAC |
| | TTAAGAAACCCAACATAATACCAAAGAACCACTTATATACCTAAAGCTGAATCTTATT |
| | ATTTATTGTATTAATAAAATGTTTATTAGAAAAATTATACCTGATTGCTGTAATCTGGA |
| | AGAATTGAAGTAGCCGATAATTTAGTGTTAAAATGTTAAATGGACTCTATAGTTGTTC |
| | ACACCCACCTAATCCTTAGAGTTAATATTACTTAATGCTTTGTGAGCTATGGTCTTTGT |
| | CAAAATGTTAGGTCATTTCCAATCTCTATGAAAATGACAAGTCTGTTCAACAAATAAA |
| | TCTCAATCTTTTTATTCTTACTACTTAAGGTAGTCCTCAAAGGCTTTGTCAATTCTAGA |
| | AAGTGTACACATAACCTTTAATAAGTCCTGAAAAACTTTCTTTTGCTTGTATTCTGTCT |
| | TTGCCCATTACAAAAAAGCATACCCAGTGGGGATGGAAATTCTGTCATCCTTGATCAC |
| | TACTGTGTGTTTGTCTAGAAGGGTGGCTGGTACATAGTAAATGTTCAATAAATAAACT |
| | CCCCCTGGCTGCTGCCTGAGCAAATTTGCATGCTTTGGGCTTTAGGTTAGAATAGACAT |
| | GAACATACATGTGTTTATGTAGGTACATTTTCACTATGCTAATGAAACAATGAAACTA |
| | TTCTAAAAATAGCCACAAGTATTGACAATTCAGATGACAGTTTTAATGTTTGTTAGTTT |
| | CTTTATTTCTTCTCTTCCCCCATTTCCTCCTCTTTCTTCTTCATATTTTTGTTTTGAAGTG |
| | GGGTAAATAATTACCTGATACATTGAGTGTAGTCCCTGAATGTATTTTGTATTACTAAT |
| | TCACAGATGCATTTGTCCTTGTAAATTAGTTGCACACAGATTTATTAGTCATTTTTGAA |
| | GAGTTAATTACACTGTTGTTTTTGATTATAAAGACAAACATGGTCATCTAAGAAAACA |
| | AAATATAAGAGGTTGTGAAAAGTAATCCAGATAATGCTTAAATCCATAGTCGGGACTC |
| | TCAAGAAAAACTATATTAATGAATGTGGTATGTTAAGAAATGGTGTTTTCTTCAGTTTT |
| | TAATATTTATGCTTTTCATTTGTATGTTTAAAACTATAATTAGGAATATCTAGTATATA |
| | TATGTGTGTGTAATTTTTCACATTTTTATTTAACATTATTATAAAAACTTTCCCTGTCAA |
| | CAAAATTAAACAAAACTCATCACTAATAATTTAATATTCTATCAAGGGATGTTCCATG |
| | ATTTACCTAATTGTTTAAAAAGTTATATATTGGGCTTCTTTTGGGGAAGTCCATTTTTT |
| | AATGAGTAGGTGATAAATGCATACATATCTGTTGTAACTCCTTTTGCTTATTTCTTCTA |
| | AATTTTTTTTTAGGCTAAGATAATTGCAATGGTTTAGAAGTCCGTTGGTGTATTTTGAA |
| | GTGTGAATGCCTATGTTAACATTTTTTTTTTACAGAACAAAATAGTTTTATTTAAGATT |
| | CTTTTCTTTTCTGTTGTATAACCACTTATAGTTCTTGTAAATCATTTTTTCCCAGGCATT |
| | GCAAAACTGGAAGAAGGAGATGAACTCCAACTTGCAATACCAAGAGAAAATGCACAA |
| | ATATCACTGGATGGAGATGTCACATTTTTTGGTGCATTGAAACTGCTGTGACCTACTTA |
| | CACCATGTCTGTAGCTATTTTCCTCCCTTTCTCTGTACCTCTAAGAAGAAAGAATCTAA |
| | CTGAAAATACCAAAAAAAAAAAAAAAAAAAAAAAAAAAAAAAGTAGTTACCATTGC |
| | CTTTTCTGTGAGCTATTTGTTTTGGTTTGCTGAAACTAGTCCAAAACAGGAAATTTAAC |
| | AGACAGCCACAGCCAAAGAGTGTCATGTGAATTACAAGAAATAGAGCCCATTTAGGG |
| | AAAGATAGAACTAGAAAGGCTTTTCATTATAATTCCATGTTGAACAATTGAGTCATAG |
| | CTTCTTATCTTGGAGGAAGGACACAATTCAAAGGGGCAGTAAGGATTTTGTAAAACGT |
| | GGCATCCATAATTTACTATGGAGCAAGTGCCCACATCTCTAGGACATTAAGACATTTA |
| | TGAGAAATCTCAGGATTCATCTTCTGTTTTTATGTTAAATGCACTCCCTCCTTTTCAGTT |
| | AACATTATAAAAAGTAAAAAATGAAAATTTTAGAAATCTTGCATTAGACACATGAAA |
| | AAATAACTAAAAGTTTAAATTTAAATATGAAACAATTTTGCTGAAAATAGTATCCATA |
| | TACTATTTAAGTCTTTTATGGTTATTTCAAGTATACAATTTCTATCTGTAATGTAATATA |
| | TTACCCACACATTTTTTTCACAGGAGAGAGAGAATATCCTCATTTGTTTATGCTCATGT |
| | GTATTTTCTATAGTGAATTTCAGAAACTTTTAATATCAGGTAATTTCAATTTATGCCTA |
| | TAAAGCATTGATTGAAAAATAACTAGAATTGTGCATATATAACACATAATCTCCAACA |
| | GAAGTTACTGAATACATTCATACTAATGTAATGTAATTTCCCTTTATTTCTTGCTCTTCT |
| | GTTTCAAACTGCTGCTATTGTAGTTTACATATCCCAACCTTTAAAAATATTCCTCTTATT |
| | AGCTTTATATTCACTTTATAGAAGTTGAGTTTTAATTAAAATTCTTGGCATCCTGAAGT |
| | ATGTCACATAGCATGTGCTCCTTATAAATATGTTGATATCTCAGAAGACAGCATCCCG |
| | GTTTTCATTTTATAAAGTACCATACTTAAGAATGCTGTAATACTTATCTTTTATAACAT |
| | GTTTCCTTCGCTTTGCTTGTCTTTTATGTCATCAGTTTTAACTGTTTACTTCATTTAACA |
| | GTTTACATCATTCAACAGTTTACTTCATTAAACAGTAGGTGGAAAAATAGATGCCAGT |
| | CTATGAAAATCTTCCCATCTATATCAAAATACTTTTCAAGGATATACTTTTCAAAACAA |
| | ACGATTTAAATTTTATGTTTAAAATATAAACTTTAGATTTAAACTTTATTTAAATATCT |
| | GGTTCCTATGATTTTGACTTCAGTAAGTTCAAATAAAATATATTTTGCAATTCATTTTT |
| | ACATTATAATTTAAAAAGAAGAAGCGATAAGTGGAGTCAGTTTCAATGCTAGGTGGG |
| | GTGGTTAATGATTTTTCTGGTGTTGCTGCTAATGTGGATTAACAAATAAAAACATTCAT |
| | TGCCTTTTGCCTCAT |

A checkpoint inhibitor may comprise an antibody, a small molecule, a protein, a peptide, an aptamer, an enzyme, or any combination thereof. A checkpoint inhibitor or a salt thereof may be a PD-1 inhibitor or a salt thereof. The PD-1 inhibitor or a salt thereof may comprise nivolumab (Opdivo®, CAS number 946414-94-4), pembrolizumab (Keytruda®, CAS number 1374853-91-4), pidilizumab (CT-011, CAS number 1036730-42-3), BGB-A31, MEDI680 (AMP-514), a salt of any one thereof, or any combination thereof. The PD-1 inhibitor or a salt thereof may be selected from the group consisting of nivolumab (Opdivo®, CAS number 946414-94-4), pembrolizumab (Keytruda®, CAS number 1374853-91-4), pidilizumab (CT-011, CAS number 1036730-42-3), BGB-A31, MEDI0680 (AMP-514), a salt of any one thereof, and any combination thereof. The PD-1 inhibitor may be nivolumab or a salt thereof. The PD-1 inhibitor may be pembrolizumab (Keytruda®, CAS number 1374853-91-4) or a salt thereof. The PD-1 inhibitor may be pidilizumab (CT-011, CAS number 1036730-42-3) or a salt thereof. The PD-1 inhibitor may be BGB-A31 or a salt thereof. The PD-1 inhibitor may be MEDI0680 (AMP-514) or a salt thereof. In some embodiments, the PD-1 inhibitor can be an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor can be AMP-224. In some embodiments, the PD-L1 inhibitor can be anti-PD-L1 antibody. In some embodiments, the anti-PD-L1 binding antagonist can be YW243.55.S70, MPDL3280A, MEDI-4736, MSB-0010718C, or MDX-1105. MDX-1105, also known as BMS-936559, can be an anti-PD-L1 antibody described in US2009/0055944A1. Antibody YW243.55.S70 (heavy and light chain variable region sequences shown in SEQ ID Nos. 20 and 21, respectively) can be an anti-PD-L1 described in US 2010/0203056.

MDX-1106, also known as MDX-1106-04, Nivolumab, ONO-4538 or BMS-936558, is an anti-PD-1 antibody described in US 2009/0217401. Merck 3745, also known as MK-3475 or SCH-900475, Lambrolizumab, or Pembrolizumab, can be an anti-PD-1 antibody described in US 2011/0008369. Pidilizumab (CT-011; Cure Tech) can be a humanized IgGlk monoclonal antibody that binds to PD-1. Pidilizumab and other humanized anti-PD-1 monoclonal antibodies are disclosed in US 2011/0117085. In other embodiments, the anti-PD-1 antibody can be pembrolizumab. AMP-224 (B7-DCIg; Amplimmune; e.g., disclosed in US 2011/0223188 and US 2013/0017199), can be a PD-L2 Fc fusion soluble receptor that blocks the interaction between PD-1 and B7-H1. Other anti-PD-1 antibodies include AMP 514.

Nivolumab (clone 5C4) and other human monoclonal antibodies that specifically bind to PD-1 are disclosed in U.S. Pat. No. 8,008,449 and US 2009/0217401. Pembrolizumab or Lambrolizumab (also referred to as MK-3475; Merck) can be a humanized IgG4 monoclonal antibody that binds to PD-1. Pembrolizumab and other humanized anti-PD-1 antibodies are disclosed in U.S. Pat. No. 8,354,509 and US 2011/0008369.

MDPL3280A (Genentech/Roche) can be a human Fc optimized IgG monoclonal antibody that binds to PD-L1. MDPL3280A and other human monoclonal antibodies to PD-L1 are disclosed in U.S. Pat. No. 7,943,743 and U.S. Publication No.: 2012/0039906. Other anti-PD-L1 binding inhibitors can include YW243.55.S70 (described in US 2010/0203056) and MDX-1105 (also referred to as BMS-936559, and, e.g., anti-PD-L1 binding agents disclosed in US 2009/0055944).

In some embodiments, the PD-1 or PD-L1 inhibitor may have the sequence of SEQ ID NO: 17, SEQ ID NO: 18, SEQ ID NO: 19, SEQ ID NO: 20, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, a fragment of any of these, a sequence having at least about: 60%, 65%, 70%, 75%, 80%, 85%, 90%, 95%, 99% or more sequence homology to any of these, or any combination thereof (Table 3B). The PD-1 or PD-L1 inhibitor may be a biosimilar, wherein the biosimilar may be truncated and may have less than 100% length of the reference compound. In some embodiments, the biosimilar that is less than 10000 length of the reference compound may still have a remaining sequence that is at least about 900 or more matching the corresponding part of the original reference sequence.

TABLE 3B

| SEQ ID NO: | Sequence |
|---|---|
| 17: exemplary | QVQLVQSGVEVKKPGASVKVSCKASGYTFTNYYMYWVRQAPGQGLEWMGGINPSNG |
| Heavy Chain | GTNFNEKFKNRVTLTTDSSTTTAYMELKSLQFDDTAVYYCARRDYRFDMGFDYWGQG |
| Sequence | TTVTVSSASTKGPSVFPLAPCSRSTSESTAALGCLVKDYFPEPVTVSWNSGALTSGVHTF |
| | PAVLQSSGLYSLSSVVTVPSSSLGTKTYTCNVDHKPSNTKVDKRVESKYGPPCPPCPAPE |
| | FLGGPSVFLFPPKPKDTLMISRTPEVTCVVVDVSQEDPEVQFNWYVDGVEVHNAKTKP |
| | REEQFNSTYRVVSVLTVLHQDWLNGKEYKCKVSNKGLPSSIEKTISKAKGQPREPQVYT |
| | LPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLGK |
| 18: exemplary | EIVLTQSPATLSLSPGERATLSCRASKGVSTSGYSYLHWYQQKPGQAPRLLIYLASYLES |
| Light Chain | GVPARFSGSGSGTDFTLTISSLEPEDFAVYYCQHSRDLPLTFGGGTKVEIKRTVAAPSVFI |
| amino acid | FPPSDEQLKSGTASVVCLLNNFYPREAKVQWKVDNALQSGNSQESVTEQDSKDSTYSL |
| Sequence | SSTLTLSKADYEKHKVYACEVTHQGLSSPVTKSFNRGEC |
| 19: exemplary | QVQLVESGGGVVQPGRSLRLDCKASGITFSNSGMHWVRQAPGKGLEWVAVIWYDGSK |
| Heavy Chain | RYYADSVKGRFTISRDNSKNTLFLQMNSLRAEDTAVYYCATNDDYWGQGTLVTVSSA |
| amino acid | STKGPSVFPLAPCSRSTSESTAALGCLVKDYFPEPVTVSWNSGALTSGVHTFPAVLQSSG |
| Sequence | LYSLSSVVTVPSSSLGTKTYTCNVDHKPSNTKVDKRVESKYGPPCPPCPAPEFLGGPSVF |
| | LFPPKPKDTLMISRTPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRW |
| | QEGNVFSCSVMHEALHNHYTQKSLSLSLGK |
| 20: exemplary | EIVLTQSPATLSLSPGERATLSCRASQSVSSYLAWYQQKPGQAPRLLIYDASNRATGIPA |
| amino acid Light | RFSGSGSGTDFTLTISSLEPEDFAVYYCQQSSNWPRTFGQGTKVEIKRTVAAPSVFIFPPS |
| Chain Sequence | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC |
| 165: exemplary | EVQLVESGGG LVQPGGSLRL SCAASGFTFS DSWIHWVRQA PGKGLEWVAW |
| Heavy Chain | ISPYGGSTYY ADSVKGRFTI SADTSKNTAY LQMNSLRAED TAVYYCARRH |
| amino acid | WPGGFDYWGQ GTLVTVSSAS TKGPSVFPLA PSSKSTSGGT AALGCLVKDY |
| sequence | FPEPVTVSWN SGALTSGVHT FPAVLQSSGL YSLSSVVTVP SSSLGTQTYI |
| | CNVNHKPSNT KVDKKVEPKS CDKTHTCPPC PAPELLGGPS VFLFPPKPKD |
| | TLMISRTPEV TCVVVDVSHE DPEVKFNWYV DGVEVHNAKT KPREEQYAST |
| | YRVVSVLTVL HQDWLNGKEY KCKVSNKALP APIEKTISKA KGQPREPQVY |
| | TLPPSREEMT KNQVSLTCLV KGFYPSDIAV EWESNGQPEN NYKTTPPVLD |
| | SDGSFFLYSK LTVDKSRWQQ GNVFSCSVMH EALHNHYTQK SLSLSPGK |
| 166: exemplary | DIQMTQSPSS LSASVGDRVT ITCRASQDVS TAVAWYQQKP GKAPKLLIYS |
| Light Chain | ASFLYSGVPS RFSGSGSGTD FTLTISSLQP EDFATYYCQQ YLYHPATFGQ |
| amino acid | GTKVEIKRTV AAPSVFIFPP SDEQLKSGTA SVVCLLNNFY PREAKVQWKV |
| sequence | DNALQSGNSQ ESVTEQDSKD STYSLSSTLT LSKADYEKHK VYACEVTHQG |
| | LSSPVTKSFN RGEC |
| 167: exemplary | QVQLVESGGG VVQPGRSLRL DCKASGITFS NSGMHWVRQA PGKGLEWVAV |
| Heavy Chain | IWYDGSKRYY ADSVKGRFTI SRDNSKNTLF LQMNSLRAED TAVYYCATND |
| amino acid | DYWGQGTLVT VSSASTKGPS VFPLAPCSRS TSESTAALGC LVKDYFPEPV |
| sequence | TVSWNSGALT SGVHTFPAVL QSSGLYSLSS VVTVPSSSLG TKTYTCNVDH |
| | KPSNTKVDKR VESKYGPPCP PCPAPEFLGG PSVFLFPPKP KDTLMISRTP |
| | EVTCVVVDVS QEDPEVQFNW YVDGVEVHNA KTKPREEQFN STYRVVSVLT |
| | VLHQDWLNGK EYKCKVSNKG LPSSIEKTIS KAKGQPREPQ VYTLPPSQEE |
| | MTKNQVSLTC LVKGFYPSDI AVEWESNGQP ENNYKTTPPV LDSDGSFFLY |
| | SRLTVDKSRW QEGNVFSCSV MHEALHNHYT QKSLSLSLGK |
| 168: exemplary | EIVLTQSPAT LSLSPGERAT LSCRASQSVS SYLAWYQQKP GQAPRLLIYD |
| Light Chain | ASNRATGIPA RFSGSGSGTD FTLTISSLEP EDFAVYYCQQ SSNWPRTFGQ |
| amino acid | GTKVEIKRTV AAPSVFIFPP SDEQLKSGTA SVVCLLNNFY PREAKVQWKV |
| sequence | DNALQSGNSQ ESVTEQDSKD STYSLSSTLT LSKADYEKHK VYACEVTHQG |
| | LSSPVTKSFN RGEC |
| 169: exemplary | EVQLLESGGG LVQPGGSLRL SCAASGFTFS SYIMMWVRQA PGKGLEWVSS |
| Heavy Chain | IYPSGGITFY ADTVKGRFTI SRDNSKNTLY LQMNSLRAED TAVYYCARIK |
| amino acid | LGTVTTVDYW GQGTLVTVSS ASTKGPSVFP LAPSSKSTSG GTAALGCLVK |
| sequence | DYFPEPVTVS WNSGALTSGV HTFPAVLQSS GLYSLSSVVT VPSSSLGTQT |
| | YICNVNHKPS NTKVDKKVEP KSCDKTHTCP PCPAPELLGG PSVFLFPPKP |
| | KDTLMISRTP EVTCVVVDVS HEDPEVKFNW YVDGVEVHNA KTKPREEQYN |
| | STYRVVSVLT VLHQDWLNGK EYKCKVSNKA LPAPIEKTIS KAKGQPREPQ |
| | VYTLPPSRDE LTKNQVSLTC LVKGFYPSDI AVEWESNGQP ENNYKTTPPV |
| | LDSDGSFFLY SKLTVDKSRW QQGNVFSCSV MHEALHNHYT QKSLSLSPGK |
| 170: exemplary | QSALTQPASV SGSPGQSITI SCTGTSSDVG GYNYVSWYQQ HPGKAPKLMI |
| Light Chain | YDVSNRPSGV SNRFSGSKSG NTASLTISGL QAEDEADYYC SSYTSSSTRV |
| amino acid | FGTGTKVTVL GQPKANPTVT LFPPSSEELQ ANKATLVCLI SDFYPGAVTV |
| sequence | AWKADGSPVK AGVETTKPSK QSNNKYAASS YLSLTPEQWK SHRSYSCQVT |
| | HEGSTVEKTV APTECS |
| 171: exemplary | EVQLVESGGG LVQPGGSLRL SCAASGFTFS RYWMSWVRQA PGKGLEWVAN |
| Heavy Chain | IKQDGSEKYY VDSVKGRFTI SRDNAKNSLY LQMNSLRAED TAVYYCAREG |
| amino acid | GWFGELAFDY WGQGTLVTVS SASTKGPSVF PLAPSSKSTS GGTAALGCLV |
| sequence | KDYFPEPVTV SWNSGALTSG VHTFPAVLQS SGLYSLSSVV TVPSSSLGTQ |
| | TYICNVNHKP SNTKVDKRVE PKSCDKTHTC PPCPAPEFEG GPSVFLFPPK |
| | PKDTLMISRT PEVTCVVVDV SHEDPEVKFN WYVDGVEVHN AKTKPREEQY |
| | NSTYRVVSVL TVLHQDWLNG KEYKCKVSNK ALPASIEKTI SKAKGQPREP |
| | QVYTLPPSRE EMTKNQVSLT CLVKGFYPSD IAVEWESNGQ PENNYKTTPP |
| | VLDSDGSFFL YSKLTVDKSR WQQGNVFSCS VMHEALHNHY TQKSLSLSPG K |
| 172: exemplary | EIVLTQSPGT LSLSPGERAT LSCRASQRVS SSYLAWYQQK PGQAPRLLIY |
| Light Chain | DASSRATGIP DRFSGSGSGT DFTLTISRLE PEDFAVYYCQ QYGSLPWTFG |
| amino acid | QGTKVEIKRT VAAPSVFIFP PSDEQLKSGT ASVVCLLNNF YPREAKVQWK |
| sequence | VDNALQSGNS QESVTEQDSK DSTYSLSSTL TLSKADYEKH KVYACEVTHQ |
| | GLSSPVTKSF NRGEC |
| 173: exemplary | EVQLLESGGV LVQPGGSLRL SCAASGFTFS NFGMTWVRQA PGKGLEWVSG |
| Heavy Chain | ISGGGRDTYF ADSVKGRFTI SRDNSKNTLY LQMNSLKGED TAVYYCVKWG |
| amino acid | NIYFDYWGQG TLVTVSSAST KGPSVFPLAP CSRSTSESTA ALGCLVKDYF |
| sequence | PEPVTVSWNS GALTSGVHTF PAVLQSSGLY SLSSVVTVPS SSLGTKTYTC |
| | NVDHKPSNTK VDKRVESKYG PPCPPCPAPE FLGGPSVFLF PPKPKDTLMI |
| | SRTPEVTCVV VDVSQEDPEV QFNWYVDGVE VHNAKTKPRE EQFNSTYRVV |
| | SVLTVLHQDW LNGKEYKCKV SNKGLPSSIE KTISKAKGQP REPQVYTLPP |
| | SQEEMTKNQV SLTCLVKGFY PSDIAVEWES NGQPENNYKT TPPVLDSDGS |
| | FFLYSRLTVD KSRWQEGNVF SCSVMHEALH NHYTQKSLSL SLGK |
| 174: exemplary | DIQMTQSPSS LSASVGDSIT ITCRASLSIN TFLNWYQQKP GKAPNLLIYA |
| Light Chain | ASSLHGGVPS RFSGSGSGTD FTLTIRTLQP EDFATYYCQQ SSNTPFTFGP |
| amino acid | GTVVDFRRTV AAPSVFIFPP SDEQLKSGTA SVVCLLNNFY PREAKVQWKV |
| sequence | DNALQSGNSQ ESVTEQDSKD STYSLSSTLT LSKADYEKHK VYACEVTHQG |
| | LSSPVTKSFN RGEC |

In some embodiments, the PD1 or PD-L1 inhibitor or a pharmaceutically acceptable salt thereof can comprise a structure, formula, analogs or derivatives of any one of structures 1-7.

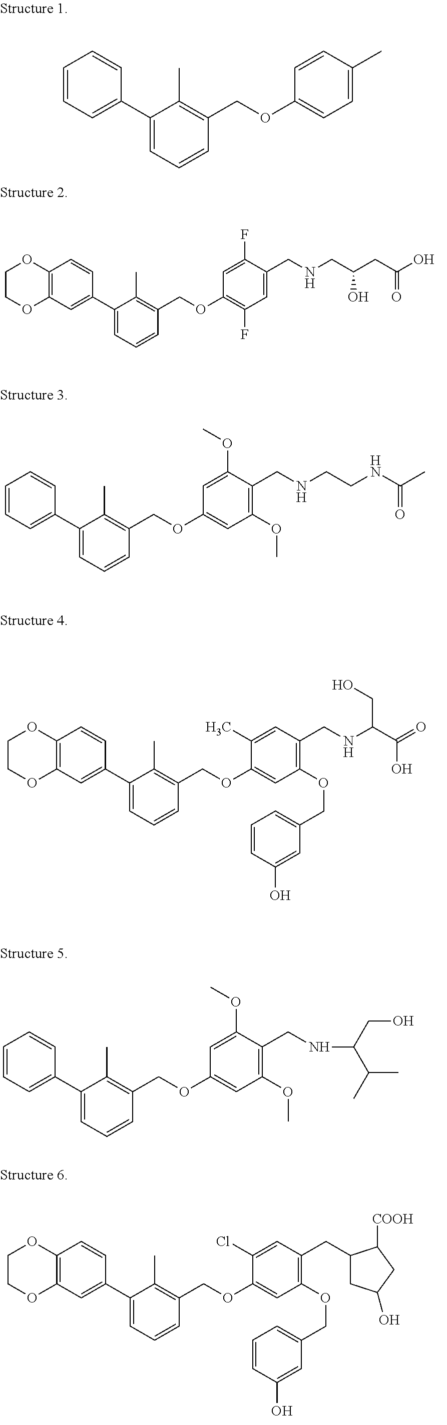 [SMILES: Cc1ccc(OCc2cccc(-c3ccccc3)c2C)cc1 ; Cc1c(COc2cc(F)c(CNC[C@@H](O)CC(=O)O)cc2F)cccc1-c1ccc2c(c1)OCCO2 ; COc1cc(OCc2cccc(-c3ccccc3)c2C)cc(OC)c1CNCCNC(C)=O ; Cc1cc(CNC(CO)C(=O)O)c(OCc2cccc(O)c2)cc1OCc1cccc(-c2ccc3c(c2)OCCO3)c1C ; COc1cc(OCc2cccc(-c3ccccc3)c2C)cc(OC)c1CNC(CO)C(C)C ; Cc1c(COc2cc(OCc3cccc(O)c3)c(CC3CC(O)CC3C(=O)O)cc2Cl)cccc1-c1ccc2c(c1)OCCO2]

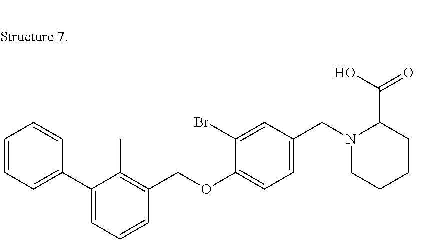 [SMILES: Cc1c(COc2ccc(CN3CCCCC3C(=O)O)cc2Br)cccc1-c1ccccc1]

In some embodiments, a biosimilar may comprise less than 100% length, less than about 90% length, less than about 80% length, less than about 70% length, less than about 60% length, or less than about 50% length to the reference compound. In some embodiment, the biosimilar may comprise a sequence that is at least about 50%, at least about 60%, at least about 70%, at least about 80%, at least about 90%, or 100% identity to the corresponding part of the original reference sequence.

In some embodiments, the checkpoint inhibitor or a salt thereof may be a PD-L1 inhibitor or a salt thereof. The PD-L1 inhibitor or a salt thereof may be selected from the group consisting of atezolizumab (Tecentriq®, CAS number 1380723-44-3), avelumab (Bavencio®, CAS number 1537032-82-8), durvalumab (Imfinzi®, CAS number 1428935-60-7), MDX-1105, MSB0010718C, a salt of any one thereof, and any combination thereof. The PD-L1 inhibitor may be atezolizumab (Tecentriq®, CAS number 1380723-44-3) or a salt thereof. The PD-L1 inhibitor may be avelumab (Bavencio®, CAS number 1537032-82-8) or a salt thereof. The PD-L1 inhibitor may be durvalumab (Imfinzi®, CAS number 1428935-60-7) or a salt thereof. The PD-L1 inhibitor may be MDX-1105 or a salt thereof. The PD-L1 inhibitor may be MSB0010718C or a salt thereof.

In some embodiments, the checkpoint inhibitor may be a CTLA4 inhibitor or a salt thereof. The CTLA4 inhibitor or a salt thereof may be selected from the group consisting of ipilimumab (Yervoy®, CAS number 477202-00-9), tremelimumab (ticilimumab, CAS number 745013-59-6), AGEN1884, a salt of any one thereof, and any combination thereof. The CTLA4 inhibitor may be ipilimumab (Yervoy®, CAS number 477202-00-9) or a salt thereof. The CTLA4 inhibitor may be tremelimumab (ticilimumab, CAS number 745013-59-6) or a salt thereof. The CTLA4 inhibitor may be AGEN1884 or a salt thereof.

In some embodiments, the checkpoint inhibitor or a salt thereof may be a LAG3 inhibitor or a salt thereof. The LAG3 inhibitor may be BMS-986016 or a salt thereof. The checkpoint inhibitor or a salt thereof may be a TIM3 inhibitor or a salt thereof. The TIM3 inhibitor or a salt thereof may be selected from the group consisting of MBG453, TSR-022, a salt of any one thereof, and any combination thereof. The TIM3 inhibitor may be MBG453 or a salt thereof. The TIM3 inhibitor may be TSR-022 or a salt thereof.

Another aspect of the current disclosure provides a method of treating a subject in need thereof. The method may comprise administering a therapeutically effective amount of at least one BAFF and/or BAFF-R inhibitor or a salt thereof to the subject; and administering at least one immune agonist agent or a salt thereof to the subject. In some embodiments, the immune agonist agent or a salt thereof may be selected from the group consisting of an agent that binds to glucocorticoid-induced tumor necrosis factor receptor-related protein (GITR), cluster of differentiation 134 (OX40), cluster of differentiation 137 (CD137), cluster of differentiation 40 (CD40), Toll-like receptor (TLR), a salt of any one thereof, and any combination thereof.

In some embodiments, the immune agonist agent may be a GITR agonist or a salt thereof. The GITR agonist or a salt thereof may be selected from the group consisting of an agent that binds to TRX518, GWN323, MEDI1873, INCAGN01876, a salt of any one thereof, and any combination thereof. The GITR agonist may be TRX518 or a salt thereof. The GITR agonist may be GWN323 or a salt thereof. The GITR agonist may be MEDI1873 or a salt thereof. The GITR agonist may be INCAGN01876 or a salt thereof.

In some embodiments, the immune agonist agent may be an OX40 agonist or a salt thereof. The OX40 agonist may be selected from the group consisting of GSK3174998, PF-04518600, MEDI6469, INCAGN01949, a salt of any one thereof, and any combination thereof. The OX40 agonist may be GSK3174998 or a salt thereof. The OX40 agonist may be PF-04518600 or a salt thereof. The OX40 agonist may be MEDI6469 or a salt thereof. The OX40 agonist may be INCAGN01949 or a salt thereof.

In some embodiments, the immune agonist agent may be a cluster of differentiation 137 (4-1BB) agonist or a salt thereof. The 4-1BB agonist may be urelumab, utomilumab, or a salt of any one thereof. The 4-1BB agonist may be isurelumab or a salt thereof. The 4-1BB agonist may be isutomilumab or a salt thereof.

In some embodiments, the immune agonist agent may be a CD40 agonist or a salt thereof. The CD40 agonist may be APX005M, CP870893, or a salt thereof. The CD40 may be APX005M or a salt thereof. The CD40 may be CP870893 or a salt thereof.

In some embodiments, the immune agonist agent may be a TLR agonist or a salt thereof. The TLR agonist may be selected from the group consisting of an agent that binds to TLR-1, TLR-2, TLR-3, TLR-4, TLR-5, TLR-6, TLR-7, TLR-8, TLR-9, TLR-13, a salt or any one thereof, and any combination thereof.

In some embodiments, the immune agonist agent may be a KIR agonist or a salt thereof. The KIR agonist may be antibody 1-7F9, or a salt thereof.

Yet another aspect of the current disclosure provides a method of treating a subject in need thereof. The method may comprise: administering a therapeutically effective amount of at least one BAFF and/or BAFF-R inhibitor or a salt thereof; and administering at least one vaccine agent or a salt thereof. The vaccine agent may be selected from the group consisting of: MAGE-3, NY-ESO-1, TRAG-3, p53, at least one or more α-actinin-4 and malic enzymes, carcinoembryonic antigen, HER2, MUC1, survivin, WT-1, PRAME, Survivin-2b, Bacillus Calmette-Guerin, MVAX, at least one or more heat shock proteins, keyhole limpet hemocyanin, interleukin-2, QS21, montanide ISA-51, granulocyte monocyte-colony stimulating factor, GVAX, GI-4000, CDX-1307, IMA910, TroVAX, CRS-207, CA-9, a salt of any one thereof, and any combination thereof.

An aspect of the current disclosure provides a method of treating a subject in need thereof. The method may comprise: administering a therapeutically effective amount of at least one BAFF and/or BAFF-R inhibitor or a salt thereof; and administering at least one oncolytic viral based agent or a salt thereof. The oncolytic viral based agent may be selected from the group consisting of: enadenotucirev, talimogene laherparepvec, reolysin, CG0070, Pexastimogene devacirepvec, cavatak, oncolytic vesicular stomatitis virus, ONCOS-102, a salt of any one thereof, and any combination thereof.

An aspect of the current disclosure provides a method of treating a subject in need thereof. The method may comprise: administering a therapeutically effective amount of at least one BAFF and/or BAFF-R inhibitor or a salt thereof; and administering at least one cell-based therapy, any cell derivative thereof, or a salt of any one thereof. The cell-based therapy may be selected from the group consisting of: at least one or more autologous lymphocytes; at least one or more genetically engineered autologous lymphocytes; at least one or more chimeric antigen receptor cells; at least one or more chimeric antigen receptor T-cells; at least one or more dendritic cell based vaccines; a salt of any one thereof, and any combination thereof.

Another aspect of the current disclosure provides a method of treating a subject in need thereof. The method may comprise: administering a therapeutically effective amount of at least one BAFF and/or BAFF-R inhibitor or a salt thereof; and administering at least one chemotherapeutic agent or a salt thereof. The chemotherapeutic agent may be an alkylating agent, an antimetabolite agent, a plant alkaloid agent, an antitumor antibiotic, a salt of any one thereof, and any combination thereof.

One aspect of the current disclosure provides for a method of treating a subject in need thereof. The method may comprise: administering a therapeutically effective amount of at least one BAFF and/or BAFF-R inhibitor or a salt thereof; and administering at least one targeted therapeutic agent or a salt thereof. The targeted therapeutic agent may be an agent that inhibits signal transduction, angiogenesis, hormone expression, or any combination thereof. The BAFF inhibitor and/or BAFF-R inhibitor may prevent binding of BAFF or a BAFF-induced ligand to a BAFF-R; may be a partial antagonist of a BAFF; may be a partial agonist of a BAFF; may be a competitive antagonist of a BAFF-R; may be a non-competitive antagonist of a BAFF-R receptor; or any combination thereof. The BAFF inhibitor may be an antibody, antigen binding fragment, a bispecific antibody, or a recombinant fusion protein.

In some embodiments, the BAFF inhibitor antibody may be human, humanized, chimeric, composite, polyclonal or monoclonal. The BAFF antibody or antigen binding fragment may block interaction of molecules to BAFF. The BAFF inhibitor may be selected from the group consisting of: tabalumab (CAS number 1143503-67-7), atacicept (CAS number 845264-92-8), RCT-18, BAFF/IL-17 bispecific antibody, ardenermin, briobacept, blisibimod (A-623, CAS number 1236126-45-6), belimumab (Benlystra®, CAS number 356547-88-1), a transmembrane activator and calcium-modulator and cyclophilin ligand interactorcept, a salt of any one thereof, and any combination thereof. The BAFF inhibitor may be selected from the group consisting of: MEDI-700, NOV-5, rGel/BLyS, a salt of any one thereof, and any combination thereof. The BAFF inhibitor may be tabalumab (CAS number 1143503-67-7) or a salt thereof. The BAFF inhibitor may be blisibimod (A-623, CAS number 1236126-45-6) or a salt thereof. The BAFF inhibitor may be belimumab (Benlystra®, CAS number 356547-88-1) or a salt thereof. The BAFF inhibitor may be atacicept (CAS number 845264-92-8).

One aspect of the current disclosure provides a method of treating a subject in need thereof. The method may comprise: administering a therapeutically effective amount of an immunotherapeutic agent or a salt thereof, wherein the immunotherapeutic agent or a salt thereof may inhibit a biological cascade selected from the group consisting of: a B-cell activating factor receptor cascade, a transmembrane activator and calcium-modulator and cyclophilin ligand interactor receptor cascade, a B-cell maturation antigen receptor cascade, and any combination thereof, wherein the immunotherapeutic agent may be co-administered with a checkpoint inhibitor.

In some embodiments, the subject may have been diagnosed with a cancer or a tumor. The method may be a method of treating a cancer or a tumor in the subject. The subject may have previously or may be currently undergoing treatment for the cancer or the tumor. The subject may have been previously treated with a checkpoint inhibitor, and the cancer or the tumor may have been at least partially refractive to the checkpoint inhibitor. In some embodiments, a method may comprise administering a therapeutically effective amount of the checkpoint inhibitor.

In some embodiments, the checkpoint inhibitor may be a salt. The checkpoint inhibitor may be selected from the group consisting of: an anti-programmed cell death protein 1 agent, an anti-programmed death ligand 1 agent, an anti-programmed death ligand 2 agent, a salt of any of these, and any combination thereof.

In some embodiments, vemurafenib (Zelboraf®, CAS number 918504-65-1) or erlotinib (Tarceva®, CAS number 183321-74-6) may be administered. In some embodiments, a method described herein may comprise administering the anti-programmed cell death protein 1 agent or a salt thereof. The anti-programmed cell death protein 1 agent or a salt thereof may be selected from the group consisting of docetaxel (Taxotere®, CAS number 114977-28-5), nivolumab (Opdivo®, CAS number 946414-94-4), pembrolizumab (Keytruda®, CAS number 1374853-91-4), a salt of any of these, and any combination thereof. In some embodiments, an agent or a salt thereof may be vemurafenib (Zelboraf®, CAS number 918504-65-1) or erlotinib (Tarceva®, CAS number 183321-74-6). The anti-programmed cell death protein 1 agent or a salt thereof may be pembrolizumab (Keytruda®, CAS number 1374853-91-4). In some embodiments, a method may comprise administering the anti-programmed death ligand 1 agent or a salt thereof.

In some embodiments, the anti-programmed death ligand 1 agent or a salt thereof may be selected from the group consisting of atezolizumab (Tecentriq®, CAS number 1380723-44-3), avelumab (Bavencio®, CAS number 1537032-82-8), durvalumab (Imfinzi®, CAS number 1428935-60-7), a salt of any of these, and any combination thereof. The immunotherapeutic agent or a salt thereof at least partially may prevent binding of a ligand to a B-cell activating factor receptor; may be a partial antagonist of a B-cell activating factor receptor; may be a partial agonist of a B-cell activating factor receptor; may be a competitive antagonist of a B-cell activating factor receptor; may be a non-competitive antagonist of a B-cell activating factor receptor; or any combination thereof. In some embodiments, the immunotherapeutic agent or a salt thereof at least partially may prevent ligand binding to a transmembrane activator and calcium-modulator and cyclophilin ligand interactor receptor; may be a partial antagonist of a transmembrane activator and calcium-modulator and cyclophilin ligand interactor receptor; may be a partial agonist of a transmembrane activator and calcium-modulator and cyclophilin ligand interactor receptor; or any combination thereof.

In some embodiments, the immunotherapeutic agent or a salt thereof at least partially may prevent ligand binding to a B-cell maturation antigen receptor; may be a partial antagonist of a B-cell maturation antigen receptor; may be a partial agonist of a B-cell maturation antigen receptor; or any combination thereof. The immunotherapeutic agent may be an antibody or a salt thereof. The immunotherapeutic agent may be a human or humanized monoclonal antibody or a salt thereof. The immunotherapeutic agent or a salt thereof may be selected from the group consisting of ardenermin, briobacept, blisibimod (A-623, CAS number 1236126-45-6), belimumab (Benlystra®, CAS number 356547-88-1), a transmembrane activator and calcium-modulator and cyclophilin ligand interactorcept, tabalumab (CAS number 1143503-67-7), atacicept, RCT-18, a salt of any of these, and any combination thereof.

In some embodiments, the immunotherapeutic agent or a salt thereof may be selected from the group consisting of MEDI-700, NOV-5, rGel/BLyS, a salt of any of these, and any combination thereof. The immunotherapeutic agent may be tabalumab (CAS number 1143503-67-7) or a salt thereof. The immunotherapeutic agent may be blisibimod (A-623, CAS number 1236126-45-6) or a salt thereof. The immunotherapeutic agent may be belimumab (Benlystra®, CAS number 356547-88-1) or a salt thereof. The immunotherapeutic agent may be atacicept (CAS number 845264-92-8) or a salt thereof.

In some embodiments, a method described herein may further comprise, before the administering: determining a count of cluster of differentiation 8 (CD8) protein, and at least one selected from the group consisting of: cluster of differentiation 19 (CD19) protein, cluster of differentiation 20 protein (CD20), cluster of differentiation 138 (CD138) protein, B-cell activating factor protein, B-cell activating factor receptor protein, paired box Pax-5 (PAX5) protein, and any combination thereof, in a cancer or a tumor or a cancer sample or a tumor sample of a subject.

In some embodiments, the at least one of: CD19, CD20, CD138, B-cell activating factor, B-cell activating factor-R, and PAX5 may be present in the cancer or the tumor or the cancer sample or the tumor sample of the subject. The at least one of: CD19, CD20, CD138, B-cell activating factor, B-cell activating factor-R, and PAX5 may be individually present in the cancer or the tumor or the cancer sample or the tumor sample of the subject in an amount ranging from about 1 protein to at least about 10,000,000 proteins.

In some embodiments, a method described herein may further comprise monitoring the treatment after the administering. The treatment may maintain at least partial remission of the cancer or the tumor, wherein a period of the remission may be at least about: 1 month, 2 months, 3 months, 6 months, 1 year, 2 years, 3 years, 4 years, 5 years, 6 years, 7 years, 8 years, 9 years, or 10 years. The treatment may comprise, after administering, delaying progression of the cancer or the tumor in the subject. The treatment may comprise, after administering, regression of the cancer or the tumor.

In some embodiments, the regression may be a reduction in mass of the cancer or the tumor, a reduction in volume of the cancer or the tumor, or both. The treatment may comprise prolonging the subject's life. The subject may have previously shown at least partial refraction to a monotherapy for the cancer or the tumor. The subject may have shown refraction to a monotherapy for the cancer or the tumor. The cancer may have metastasized from a first location of the subject to a second location of the subject. The cancer at the first location of the body may be less than fully responsive to the monotherapy, and wherein the cancer at the second location of the body may be responsive to the treatment. The checkpoint inhibitor and the immunotherapeutic agent may be synergistic. The synergy on the cancer or the tumor may be at least about 10% more than an additive effect.

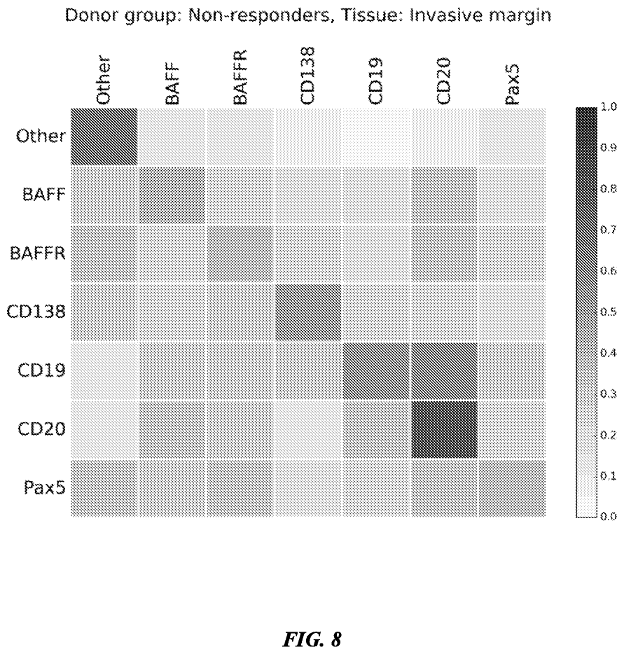

In some embodiments, a heatmap of a cancer sample or a tumor sample of the subject may show a profile substantially similar to that of FIG. 8.

In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof may be administered concurrently. In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof may be administered sequentially. In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof may be administered in different formulations within a same treatment schedule.

In some embodiments, the anti-programmed cell death protein 1 agent, the anti-programmed death ligand 1 agent, the anti-programmed death ligand 2 agent, or a salt thereof or any combination thereof, may be administered at least once a week. In some embodiments, the anti-programmed cell death protein 1 agent, the anti-programmed death ligand 1 agent, the anti-programmed death ligand 2 agent, or a salt thereof or any combination thereof, may be administered at least once a day. In some embodiments, the anti-programmed cell death protein 1 agent, the anti-programmed death ligand 1 agent, the anti-programmed death ligand 2 agent, or a salt thereof or any combination thereof, may be administered at least once during a treatment schedule.

In some embodiments, the immunotherapeutic agent or a salt thereof may be administered at least once a week. In some embodiments, the immunotherapeutic agent or a salt thereof may be administered at least once a day. In some embodiments, the immunotherapeutic agent or a salt thereof may be administered at least once during a treatment schedule. In some embodiments, the immunotherapeutic agent or a salt thereof may be administered in an amount from about 0.1 mg to about 1,000 mg per kg body weight. In some embodiments, the immunotherapeutic agent or a salt thereof may be administered in an amount from about 0.1 mg to about 100 mg per kg body weight. In some embodiments, the immunotherapeutic agent or a salt thereof may be administered in an amount from about 1 mg to about 50 mg per kg body weight.

In some embodiments, the anti-programmed cell death protein 1 agent, the anti-programmed death ligand 1 agent, the anti-programmed death ligand 2 agent, or a salt thereof or any combination thereof, may be administered in an amount from about 0.1 mg to about 1,000 mg per kg body weight. In some embodiments, the anti-programmed cell death protein 1 agent, the anti-programmed death ligand 1 agent, the anti-programmed death ligand 2 agent, or a salt thereof or any combination thereof, may be administered in an amount from about 1 mg to about 100 mg per kg body weight. In some embodiments, the anti-programmed cell death protein 1 agent, the anti-programmed death ligand 1 agent, the anti-programmed death ligand 2 agent, or a salt of any one thereof, or any combination thereof, may be individually present as a pharmaceutical formulation. The pharmaceutical formulation may be in unit dose form. The pharmaceutical formulation may further comprise a pharmaceutically-acceptable excipient.

In some cases, a method may comprise treating the cancer, wherein the cancer may be prostate cancer, testicular cancer, breast cancer, brain cancer, pancreatic cancer, colon cancer, thyroid cancer, stomach cancer, lung cancer, melanoma, multiple myeloma, Hodgkin's lymphoma, ovarian cancer, or any combination thereof.

In one aspect, the present disclosure provides a method of selecting a therapeutic regimen. The method may comprise: determining the presence or absence of cluster of differentiation 8 (CD8) protein, and at least one of: B-cell activating factor (BAFF) protein, B-cell activating factor receptor (BAFF-R) protein, and paired box Pax-5 (PAX5) protein in a cancer or a tumor of a subject or a cancer sample or a tumor sample of the subject; if CD8 protein and at least one of B-cell activating factor, B-cell activating factor-R, and PAX5 may be present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof; if CD8 protein and at least one of B-cell activating factor, B-cell activating factor-R, and PAX5 is not present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof.

In some embodiments, the method may further comprise determining whether cluster of differentiation 19 (CD19) protein, cluster of differentiation 20 (CD20) protein, cluster of differentiation 138 (CD138) protein, or any combination thereof may be present. The method may further comprise treating the subject.

In some embodiments, the treating may comprise: administering a therapeutically effective amount of an immunotherapeutic agent or a salt thereof, wherein the immunotherapeutic agent or a salt thereof may inhibit a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof, wherein the immunotherapeutic agent may be co-administered with a checkpoint inhibitor.

In some embodiments, the subject may have been diagnosed with a cancer or a tumor. The subject may have previously or may be currently undergoing treatment for the cancer or the tumor. The method may further comprise administering a therapeutically effective amount of the checkpoint inhibitor.

In some embodiments, the checkpoint inhibitor may be selected from the group consisting of an anti-programmed cell death protein 1 agent, an anti-programmed death ligand 1 agent, an anti-programmed death ligand 2 agent, a salt of any of these, and any combination thereof. The method may further comprise administering the anti-programmed cell death protein 1 agent of a salt thereof.

In some embodiments, the anti-programmed cell death protein 1 agent or a salt thereof may be selected from the group consisting of: docetaxel (Taxotere®, CAS number 114977-28-5), nivolumab (Opdivo®, CAS number 946414-94-4), pembrolizumab (Keytruda®, CAS number 1374853-91-4), a salt of any of these, and any combination thereof. The anti-programmed cell death protein 1 agent or a salt thereof may be pembrolizumab (Keytruda®, CAS number 1374853-91-4). The immunotherapeutic agent may be tabalumab (CAS number 1143503-67-7) or a salt thereof. The immunotherapeutic agent may be belimumab (Benlystra®, CAS number 356547-88-1) or a salt thereof. The immunotherapeutic agent may be atacicept (CAS number 845264-92-8) or a salt thereof. In some embodiments, the cancer or the tumor may be prostate cancer, testicular cancer, breast cancer, brain cancer, pancreatic cancer, colon cancer, thyroid cancer, stomach cancer, lung cancer, ovarian cancer, or any combination thereof. The cancer or the tumor may contact or be substantially adjacent to a blood vessel. In some embodiments, the cancer or the tumor may be present in the interior of a blood vessel.

In one aspect, the present disclosure provides a method of selecting a therapeutic regimen. The method may comprise: determining a count of B cells, T cells, and plasma cells in a cancer or a tumor of a subject or a cancer sample or a tumor sample of the subject; if a count of T cells in the cancer or the tumor or the cancer sample or the tumor sample may be greater than about 500 cells/mm² and/or a count of B cells may be less than about 150 cells/mm², selecting a therapeutic regimen comprising an anti-programmed cell death protein 1 agent, an anti-programmed death ligand 1 agent, an anti-programmed death ligand 2 agent, or a salt thereof or any combination thereof, if a count of T cells in the cancer or the tumor or the cancer sample or the tumor sample may be greater than about 300 cells/mm² and/or a count of B cells, a count of plasma cells, or a combination of B cells and plasma cells may be greater than about 100 cells/mm², selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof.

In some embodiments, a method further may comprise conveying a result via a communication medium.

In one aspect, the current disclosure provides a method of treating or maintaining remission of a cancer or a tumor in a subject. The method may comprise: administering an anti-programmed cell death protein 1 agent, an anti-programmed death ligand 1 agent, an anti-programmed death ligand 2 agent, or a salt thereof or any combination thereof, if a percentage of T cells in a cancer sample or a tumor sample of a subject may be greater than about 10% and/or a percentage of B cells may be less than about 5%, or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a percentage of T cells in a cancer or a tumor sample of the subject may be less than about 10% and a percentage of B cells, a percentage of plasma cells, or a combination of the percentage of B cells and/or the percentage of plasma cells may be greater than about 10% in the cancer sample of the subject.

In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following may be observed when compared to a count prior to treatment: a percentage of T cells, a percentage of B cells, and a percentage of plasma cells. The cancer or the tumor may be prostate cancer, testicular cancer, breast cancer, brain cancer, pancreatic cancer, colon cancer, thyroid cancer, stomach cancer, lung cancer, ovarian cancer, or any combination thereof.

In some embodiments, a sample may be obtained from a human from the peripheral blood, peripheral venous blood, peripheral arterial blood, peripheral whole blood monocytes, peripheral mononuclear cell enriched monocytes, red blood cell lysate of whole peripheral blood, serum, plasma, tears, hair, sputum, bronchoalveoli, cerebrospinal fluid, pericardial fluid, pleural fluid, peritoneal fluid, synovial fluid, vaginal fluid, urethral fluid, pericarditis fluid, pleural effusion fluid, ascites fluid, saliva, sweat, tumor, lymph, lymphatic vessels, lymph node tissue, adenoid tissue, spleen, spleen cells, cancer tissue, or any combination thereof.

A sample may comprise cells that may be intact, cells that may be dissociated, extracellular products that may be cell-derived, or any combination thereof.

Extracellular products may be derived from cells that may be dissociated from cells and extracellular in form and may comprise nucleic acids, proteins, lipids, carbohydrates, nanovesicles, microvesicles, glycated end-products, enzymes, chemical products of metabolism, chemical by-products of metabolism, cations, and anions.

### Diagnostic Methods

In some embodiments, a method of the current disclosure may comprise selecting a therapeutic regimen. A method of selecting a therapeutic regimen may comprise: determining the presence or absence of cluster of differentiation 8 (CD8) protein, and at least one of: B-cell activating factor (BAFF) protein, B-cell activating factor receptor (BAFF-R) protein, and paired box Pax-5 (PAX5) protein in a cancer or a tumor of a subject or a cancer sample or a tumor sample of the subject; if CD8 protein and at least one of B-cell activating factor, B-cell activating factor-R, and PAX5 may be present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof; if CD8 protein and at least one of B-cell activating factor, B-cell activating factor-R, and PAX5 is not present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof.

The method may further comprise determining the presence or absence of a number of proteins, or cell-surface proteins, wherein the proteins may be, but may be not limited to, cluster of differentiation 19 (CD19) protein, cluster of differentiation 20 (CD20) protein, cluster of differentiation 138 (CD138) protein, or any combination thereof.

The method may be a method of treatment, wherein the treatment may comprise: administering a therapeutically effective amount of an immunotherapeutic agent or a salt thereof, wherein the immunotherapeutic agent or a salt thereof inhibits a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof, wherein the immunotherapeutic agent may be co-administered with a checkpoint inhibitor.

In some embodiments, the checkpoint inhibitor may be selected from the group consisting of an anti-programmed cell death protein 1 agent, an anti-programmed death ligand 1 agent, an anti-programmed death ligand 2 agent, a salt of any of these, and any combination thereof. The method may comprise administering the anti-programmed cell death protein 1 agent of a salt thereof. The anti-programmed cell death protein 1 agent or a salt thereof may be selected from the group consisting of docetaxel (Taxotere®, CAS number 114977-28-5), nivolumab (Opdivo®, CAS number 946414-94-4), pembrolizumab (Keytruda®, CAS number 1374853-91-4), a salt of any of these, and any combination thereof. The anti-programmed cell death protein 1 agent or a salt thereof may be pembrolizumab (Keytruda®, CAS number 1374853-91-4). The immunotherapeutic agent may be tabalumab (CAS number 1143503-67-7) or a salt thereof. The immunotherapeutic agent may be belimumab (Benlystra®, CAS number 356547-88-1) or a salt thereof. The immunotherapeutic agent may be atacicept (CAS number 845264-92-8) or a salt thereof.

In some embodiments, a method of the current disclosure is a method of selecting a therapeutic regimen. The method may comprise: determining a count of B cells, T cells, and plasma cells in a cancer or a tumor of a subject or a cancer sample or a tumor sample of the subject; if a count of T cells in the cancer or the tumor or the cancer sample or the tumor sample may be greater than about 500 cells/mm² and/or a count of B cells is less than about 150 cells/mm², selecting a therapeutic regimen comprising an anti-programmed cell death protein 1 agent, an anti-programmed death ligand 1 agent, an anti-programmed death ligand 2 agent, or a salt thereof or any combination thereof, if a count of T cells in the cancer or the tumor or the cancer sample or the tumor sample may be greater than about 300 cells/mm² and/or a count of B cells, a count of plasma cells, or a combination of B cells and plasma cells may be greater than about 100 cells/mm², selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof. In some embodiments, a method of the current disclosure may comprise conveying a result via a communication medium.

In another aspect, the current disclosure provides a method of treating or maintaining remission of a cancer or a tumor in a subject. The method may comprise: administering an anti-programmed cell death protein 1 agent, an anti-programmed death ligand 1 agent, an anti-programmed death ligand 2 agent, or a salt thereof or any combination thereof, if a percentage of T cells in a cancer sample or a tumor sample of a subject may be greater than about 10% and/or a percentage of B cells may be less than about 5%, or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a percentage of T cells in a cancer or a tumor sample of the subject may be less than about 10% and/or a percentage of B cells, a percentage of plasma cells, or a combination of the percentage of B cells and the percentage of plasma cells may be greater than about 10% in the cancer sample of the subject.

In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following may be observed when compared to a count prior to treatment: a percentage of T cells, a percentage of B cells, and a percentage of plasma cells. The cancer or the tumor may be prostate cancer, testicular cancer, breast cancer, brain cancer, pancreatic cancer, colon cancer, thyroid cancer, stomach cancer, lung cancer, ovarian cancer, or any combination thereof.

In another aspect, the current disclosure provides a method of assessing a likelihood of a subject having a cancer or a tumor exhibiting a clinically beneficial response to treatment. The method may comprise: assessing a count of T cells, B cells, and plasma cells in a cancer or a tumor sample of a subject; and calculating, using a computer system, a probability of treatment responsiveness based on (1) a ratio between the count of T cells and a reference count of T cells, (2) a ratio between the count of B cells and a reference count of B cells, and/or (3) a ratio between the count of plasma cells and a reference count of plasma cells. The method may further comprise designating the subject as having a high probability of exhibiting a clinically beneficial response to treatment if the ratio between the count of B cells and the reference count of B cells is less than 1. The method may further comprise designating the subject as having a high probability of exhibiting a clinically beneficial response to treatment if the ratio between the count of plasma cells and the reference count of plasma cells is less than 1. After the administration of the anti-programmed cell death protein 1 agent, the anti-programmed death ligand 1 agent, the anti-programmed death ligand 2 agent, or a salt thereof or any combination thereof, a decrease in at least one of the following may be observed when compared to a count prior to treatment: a count of T cells, a count of B cells, and a count of plasma cells.

In some embodiments, a method may comprise surgery, radiation therapy, or administering a pharmaceutical agent, wherein the pharmaceutical agent may be an immunotherapeutic agent, a chemotherapeutic agent, a targeted therapeutic agent, a hormonal therapy agent, cell-based therapy agent, radiation therapy agent, or any salt thereof, or any combination thereof.

In some embodiments, a method may determine a count of cluster of differentiation 8 (CD8) protein, a count of cluster of differentiation 19 (CD19) protein, a count of cluster of differentiation 20 protein (CD20), a count of cluster of differentiation 138 (CD138) protein, a count of B-cell activating factor protein, a count of B-cell activating factor receptor protein, a count of paired box Pax-5 (PAX5) protein, or any combination thereof, in a cancer or a tumor or a cancer sample or a tumor sample of a subject.

In some embodiments, at least one of a count of cluster of differentiation 19 (CD19) protein, a count of cluster of differentiation 20 protein (CD20), a count of cluster of differentiation 138 (CD138) protein, a count of B-cell activating factor protein, a count of B-cell activating factor receptor protein, a count of paired box Pax-5 (PAX5) protein, or any combination thereof, may be present in a cancer or a tumor or a cancer sample or a tumor sample of a subject. At least one of CD19, CD20, CD138, B-cell activating factor, B-cell activating factor-R, and PAX5 may be individually present in a cancer or a tumor or a cancer sample or a tumor sample of a subject in an amount ranging from about 1 protein to at least about 10,000,000 proteins. An amount of protein in a cancer or a tumor or a cancer sample or a tumor sample may range between about 1 protein to about 100,000,000 proteins.

In one aspect, the current disclosure may provide a method of assessing a likelihood of a subject having a cancer or a tumor exhibiting a clinically beneficial response to treatment. The method may comprise: assessing a count of T cells, B cells, and plasma cells in a cancer or a tumor sample of a subject; and calculating, using a computer system, a probability of treatment responsiveness based on (1) a ratio between the count of T cells and a reference count of T cells, (2) a ratio between the count of B cells and a reference count of B cells, and/or (3) a ratio between the count of plasma cells and a reference count of plasma cells.

In some embodiments, the method further may comprise designating the subject as having a high probability of exhibiting a clinically beneficial response to treatment if the ratio between the count of B cells and the reference count of B cells may be less than 1. The method may further comprise designating the subject as having a high probability of exhibiting a clinically beneficial response to treatment if the ratio between the count of plasma cells and the reference count of plasma cells may be less than about 1.

In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following may be observed when compared to a count prior to treatment: a count of T cells, a count of B cells, and a count of plasma cells. The cancer or the tumor may be prostate cancer, testicular cancer, breast cancer, brain cancer, pancreatic cancer, colon cancer, thyroid cancer, stomach cancer, lung cancer, ovarian cancer, or any combination thereof. The method may further comprise surgery, radiation therapy, or administering a pharmaceutical agent, wherein the pharmaceutical agent may be an immunotherapeutic agent, a chemotherapeutic agent, a targeted therapeutic agent, a hormonal therapy agent, cell-based therapy agent, radiation therapy agent, any salt thereof, or any combination thereof.

In another aspect, the current disclosure provides a method of treating a cancer or a tumor in a subject. The method may comprise: administering an anti-programmed cell death protein 1 agent, an anti-programmed death ligand 1 agent, an anti-programmed death ligand 2 agent, or a salt thereof or any combination thereof, if a cancer or a tumor sample of a subject contains at least one cell contains a cluster of differentiation 8 (CD8) protein and no cell contains a cluster of differentiation 19 (CD19) protein or a cluster of differentiation 138 (CD138) protein; or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a cancer or a tumor sample of a subject contains at least one cell contains a CD8 protein and at least one cell contains a CD19 or CD138 protein.

In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following may be observed when compared to a count prior to treatment: a count of T cells, a count of B cells, and a count of plasma cells. After the administration of the anti-programmed cell death protein 1 agent, the anti-programmed death ligand 1 agent, the anti-programmed death ligand 2 agent, or a salt thereof or any combination thereof, and an immunotherapeutic agent or a salt thereof, a decrease in at least one of the following may be observed when compared to a count prior to treatment: a count of T cells, a count of B cells, and a count of plasma cells.

In some embodiments, a decrease in a count of T cells, a decrease in a count of B cells, a decrease in a count of plasma cells, or any combination thereof, may be observed. The method may be a method of treating a cancer or a tumor in the subject. The cancer or the tumor may be prostate cancer, testicular cancer, breast cancer, brain cancer, pancreatic cancer, colon cancer, thyroid cancer, stomach cancer, lung cancer, ovarian cancer, or any combination thereof.

In one aspect, the current disclosure provides a method of treating a cancer or a tumor in a subject. The method may comprise administering a therapeutically effective amount of a pharmaceutical agent and an immunotherapeutic agent. The presence of the CD8 protein, the CD19 protein, the CD20 protein, the CD139 protein, the B-cell activating factor protein, the B-cell activating factor-R protein, and the PAX5 protein may be determined by staining, imaging after staining, microscopy, or any combination thereof. The staining may comprise binding of an antibody to the protein. The staining may further comprise binding of a second antibody to a first antibody, wherein the second antibody may contain a fluorescence marker.

In some embodiments, the method may be repeated after administration of the therapeutic. The cancer sample or the tumor sample may comprise at least one cell comprising a CD8 protein and at least one cell comprising a CD19 protein, wherein the CD8 protein and the CD19 protein may have a distance apart of at most about 100 microns. The cancer sample or the tumor sample may comprise at least one cell comprising a CD8 protein and at least one cell comprising a CD19 protein, wherein the CD8 protein-containing cell and the CD19 protein-containing cell may have a distance apart of at most about 100 microns.

In some embodiments, a method described herein may comprise determining the number of aggregates of cells within a tumor sample or cancer sample of a subject. An aggregate of cells may be a cluster of cells. An aggregate may comprise at least about: 1, 2, 3, 4, 5, 10, 15, 20, 30, 40, 50, 60, 70, 80, 90, 100, 200, 300, 400, 500, 600, 700, 800, 900, 1000, 10,000, 100,000, 1,000,000 or more cells. In some embodiments, an aggregate of cells may comprise from about 1 cell to about 100 cells; from about 10 cells to about 50 cells; or from about 20 cells to about 40 cells.

In some embodiments, an aggregate of cells may be an aggregate of T-cells. In some embodiments, an aggregate of cells may be an aggregate of B-cells. In some embodiments, an aggregate of cells may comprise T-cells, B-cells, or a combination thereof. In some embodiments, a type of cell, or a cell type, may refer to a B-cell. In some embodiments, a type of cell, or a cell type, may refer to a T-cell.

In some embodiments, an aggregate may comprise a cell of hematopoietic lineage. An aggregate may comprise myeloid-derived cells, monocytes, macrophages, dendritic cells, mast cells, granulocytes, neutrophils, eosinophils, basophils, megakaryocytes, lymphoid-derived cells, NK-cells, T-cells, CD4, CD8, Gamma/delta T-cells, B-cells, plasma cells, or any combination thereof.

In some embodiments, the number of aggregates may be determined by: immunoassays, polymerase chain reaction, sequencing, including next generation sequencing, flow cytometry, or any combination thereof. The number of aggregates may be determined by: immunohistochemistry—chromogenic based, quantitative-based on the cellular level, quantitative-based on the single pixel level, immunohistochemistry—fluorescence-based, quantitative-based on the cellular level, quantitative-based on the single pixel level, RNA in situ hybridization—chromogenic based, quantitative-based on the dot level, quantitative-based on the single pixel level, RNA in situ hybridization—fluorescent based, quantitative-based on the dot level, quantitative-based on the single pixel level, multiplexed ion beam based imaging methods, or any combination thereof. Assays may be performed in single or multiplex formats. An aggregate may be detected using a nuclear counterstain.

Assay reagents may include: antibody-based reagents, primary antibody+secondary antibody, detectable label: enzymatic, chromogenic-based, detectable label: enzymatic, fluorescent-based, primary antibody+oligonucleotide, detectable label: complementary hybridization+PCR amplification, detectable label: complementary hybridization without PCR amplification, RNA-based, DNA-based reagents, or any combination thereof.

Proximity of cells may be defined as the closest distance between an edge of a first cell and an edge of a second cell. Proximity of cells may be defined as at least about: 0.01 micrometer (μm), 0.1, 0.5, 1, 2, 3, 4, 5, 6, 7, 8, 9, 10, or more in distance. A first cell may be proximal in distance to a second cell, wherein the distance may be from about 0.01 μm to about 10 μm; from about 0.1 μm to about 7 μm; or from about 1 μm to about 5 μm. In some embodiments, a first cell may be proximal in distance to a second cell, wherein the distance between the two cells may be less than about: 1 μm, 0.1 μm, or 0.01 μm. In some cases, the distance between the two cells may be small such that the cell walls may be in contact with each other.

In some embodiments, a tumor or cancer may be classified as a Type I resistant tumor or cancer or a Type II resistant tumor or cancer. A Type I resistant tumor or cancer may possess low counts of CD8 when imaged both before and during anti-PDL1 single agent treatment. A Type II resistant tumor or cancer may possess high counts of CD8 when imaged both before and during anti-PDL1 single agent treatment.

If a tumor or cancer is classified as a Type II resistant tumor or cancer, then a single agent treatment may not be as therapeutically effective as desired. If a tumor or cancer may be classified as a Type II resistant tumor or cancer, then a combination therapy, or a therapeutic regimen comprising more than one active agent, may be administered. A Type II resistant tumor or cancer may be administered a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof.

One embodiment provides a diagnostic method wherein a subject is identified as resistant to an immunotherapy comprising a single agent, such as an immune checkpoint protein inhibitor, by determining expression levels of one or more genes that code for proteins associated with a T-cell inflamed phenotype of a tumor microenvironment and correlating said expression with that of a gene coding for BAFF protein or a BAFF-receptor protein. One embodiment provides a diagnostic method wherein a subject is identified as resistant or responsive to an immunotherapy comprising a single agent, such as an immune checkpoint protein inhibitor, by determining expression levels of a gene that code for BAFF protein or a BAFF-receptor protein, or a variant thereof. In some embodiments, a diagnostic method is provided wherein a subject is identified as responsive to a combination therapy comprising an agent that regulates activity of BAFF protein or a BAFF receptor, such as a BAFF inhibitor, a BAFF-receptor (BAFF-R) inhibitor, or a salt thereof, or any combination thereof. Non-limiting examples of such BAFF or BAFF-R inhibitors are provided in various embodiments of this disclosure. In some embodiments, a monitoring method can be provided wherein a subject may be identified as a suitable candidate for continuing treatment with an immunotherapy comprising a single agent or with a combination therapy comprising an immunotherapy and an agent that regulates activity of a BAFF protein or a BAFF-receptor protein. In some embodiment, a method described herein can be repeated multiple times to monitor a subject.

The above mentioned expression levels can be in some instances determined in a biological sample isolated from said patient. The biological sample, in some example, may be from a tumor microenvironment of a subject. Exemplary genes that can be representatives of a T cell inflamed phenotype are listed in Table 4. In some embodiments, exemplary genes that can be representative of a T cell inflamed signature can comprise any 1, 2, 3, 4, 5, 6, 7, 8, 9, 10, 15, 20, 25, 30 or more genes listed in Table 4. In some cases, a method described herein can further comprise measuring expression levels of one or more genes listed in Table 5, (housekeeping genes). This disclosure contemplates variants, fragments, truncated, alternatively spliced, or mutated versions of the genes listed in Table 4 (see also sequences in Table 7) and Table 5 and the proteins encoded therefrom. One or more genes listed in Table 4 can be used to assess an inflammation status of a tumor, such as a T-cell inflamed signature. In some cases, one or more genes listed in Table 4 and a gene that codes for a BAFF protein, a BAFF-receptor protein, or a variant thereof can be used to assess a tumor signature, such as a T-cell inflamed signature. In various examples, a tumor's inflamed status, as determined by said T-cell inflamed signature, can further be used to determine a suitable therapeutic regimen for treating said tumor.

TABLE 4

| Gene name | TCGA RNASeq Identifier |
|---|---|
| IL2RG | ENSG00000147168.11 |
| CXCR6 | ENSG00000172215.5 |
| CD3D | ENSG00000167286.8 |
| CD2 | ENSG00000116824.4 |
| ITGAL | ENSG00000005844.16 |
| TAGAP | ENSG00000164691.15 |
| CIITA | ENSG00000179583.16 |
| HLA-DRA | ENSG00000204287.12 |
| PTPRC | ENSG00000081237.17 |
| CXCL9 | ENSG00000138755.5 |
| CCL5 | ENSG00000271503.4 |
| NKG_7 | ENSG00000105374.8 |
| GZMA | ENSG00000145649.7 |
| PRF1 | ENSG00000180644.6 |
| CCR5 | ENSG00000160791.13 |
| CD3E | ENSG00000198851.8 |
| GZMK | ENSG00000113088.5 |
| IFNG | ENSG00000111537.4 |
| HLA-E | ENSG00000204592.8 |
| GZMB | ENSG00000100453.11 |
| PDCD1 | ENSG00000188389.9 |
| SLAMF6 | ENSG00000162739.12 |
| CXCL13 | ENSG00000156234.7 |
| CXCL10 | ENSG00000169245.5 |
| IDO1 | ENSG00000131203.11 |
| LAG3 | ENSG00000089692.7 |
| STAT1 | ENSG00000115415.17 |
| CXCL11 | ENSG00000169248.11 |
| CD8A | ENSG00000153563.14 |
| CCL2 | ENSG00000108691.8 |
| CCL3 | ENSG00000277632.1 |
| CCL4 | ENSG00000275302.1 |
| ICOS | ENSG00000163600.11 |
| IRF1 | ENSG00000125347.12 |
| HLA-DMA | ENSG00000204257.13 |
| HLA-DMB | ENSG00000242574.7 |
| HLA-DOA | ENSG00000204252.11 |
| HLA-DOB | ENSG00000241106.5 |

TABLE 5

| Gene name | TCGA RNASeq Identifier |
|---|---|
| FAU | ENSG00000149806.9 |
| IMP3 | ENSG00000177971.8 |
| MRPL1 | ENSG00000169288.16 |
| MRPL20 | ENSG00000242485.4 |
| MRPS2 | ENSG00000122140.9 |
| MRPS30 | ENSG00000112996.8 |
| MRTO4 | ENSG00000053372.4 |
| NHP2 | ENSG00000145912.7 |
| NSA2 | ENSG00000164346.8 |
| RPL10 | ENSG00000147403.15 |
| RPL22L1 | ENSG00000163584.16 |
| RPLP0 | ENSG00000089157.14 |
| RPP38 | ENSG00000152464.13 |
| RPS10 | ENSG00000124614.12 |
| RPS27L | ENSG00000185088.11 |
| RSL1D1 | ENSG00000171490.11 |
| SNU13 | ENSG00000100138.12 |
| UBA52 | ENSG00000221983.6 |
| POLR1A | ENSG00000068654.14 |
| POLR2A | ENSG00000181222.13 |
| POLR3A | ENSG00000148606.11 |
| TWISTNB | ENSG00000105849.5 |
| ACLY | ENSG00000131473.15 |
| ACO1 | ENSG00000122729.17 |
| CS | ENSG00000062485.17 |
| DLAT | ENSG00000150768.14 |
| DLD | ENSG00000091140.11 |
| DLST | ENSG00000119689.13 |
| FH | ENSG00000091483.6 |
| IDH1 | ENSG00000138413.12 |
| MDH1 | ENSG00000014641.16 |
| OGDH | ENSG00000105953.13 |
| PC | ENSG00000173599.12 |
| PDHA1 | ENSG00000131828.12 |
| SDHA | ENSG00000073578.15 |
| SUCLA | ENSG00000136143.12 |

The expression levels can be determined using any sequencing based techniques, including but not limited to RNA-seq, methods that utilize bisulfite conversion (such as MethylC-seq8 and Reduced Representation Bisulfite Sequencing, or RRBS9), methods that utilize enrichment of methylated DNA (Methylated DNA Immunoprecipitation sequencing, or MeDIP-seq10, 11 and Methylated DNA Binding Domain sequencing, or MBD-seq12), qPCR was performed using the Fluidigm Biomark system, serial analysis gene expression technology, targeted RNA sequencing, mRNA sequencing, total RNA-seq. Any other appropriate method can be used to determine expression levels, such as, for example, immunohistochemistry, immunofluorescence, multiplexed ion beam imaging based methods. MIBI can be a mass spectrometry based imaging platform wherein secondary ion mass spectrometry can be used to image antibodies tagged with isotopically pure elemental metal reporters, instead of antibodies tagged with fluorophores or enzyme reporters that generate colored pigments.

Following determination of expression levels of genes that are associated with T cell inflamed phenotype of a tumor microenvironment, and that of BAFF or BAFF-receptor coding genes, said expression levels can be correlated using a correlation technique, such as a t-test, a paired t-test, an analysis of variance (ANOVA), a repeated measures ANOVA, a simple linear regression, a nonlinear regression, a multiple linear regression, a multiple nonlinear regression, a Wilcoxon signed-rank test, a MannWhitney test, a Kruskal-Wallis test, a Friedman test, a Spearman rank order correlation coefficient, a Kendall Tau analysis, or a nonparametric based test, such as a regression test. In some embodiments, where BAFF gene expression and T cell inflamed signature in a tumor microenvironment positively correlates, a subject may benefit from a therapeutic method disclosed herein. For example, a treatment comprising a BAFF inhibitor or a BAFF receptor inhibitor.

### Pharmaceutical Compositions

Provided herein, in some embodiments, may be compositions comprising a therapeutically effective amount of an immunotherapeutic agent and a checkpoint inhibitor.

In some embodiments, an inhibitor may refer to an agent or a therapy that may be an agonist, an antagonist, a partial agonist, a partial antagonist, or any combination thereof.

In some embodiments, an immunotherapeutic agent or a salt thereof may inhibit a biological cascade selected from the group consisting of: a B-cell activating factor receptor cascade, a transmembrane activator and calcium-modulator and cyclophilin ligand interactor receptor cascade, a B-cell maturation antigen receptor cascade, and any combination thereof.

A checkpoint inhibitor may be an anti-programmed cell death protein 1 agent, an anti-programmed death ligand 1 agent, an anti-programmed death ligand 2 agent, a salt of any of these, and any combination thereof.

An anti-programmed cell death protein 1 (PD-1) agent may be an agent that inhibits or at least partially inhibits the binding of the PD-1 cell surface receptor to a ligand, wherein the ligand may be anti-programmed death ligand 1 (PD-L1) or anti-programmed death ligand 2 (PD-L2). While not wishing to be bound by theory, inhibition of the binding of the PD-1 receptor to a ligand may activate an immune response.

An immunotherapeutic agent may be an agent that at least partially blocks a B-cell activating factor (BAFF) receptor cascade.

In some embodiments, the immunotherapeutic agent or salt thereof may inhibit a B-cell activating factor receptor (BAFF) cascade, a transmembrane activator and calcium-modulator and cyclophilin ligand interactor receptor (TACI) cascade, a B-cell maturation antigen receptor (BCMA) cascade, or any combination thereof.

In some embodiments, an immunotherapeutic agent or a salt thereof at least partially may prevent binding of a ligand to a B-cell activating factor receptor; may be a partial antagonist of a B-cell activating factor receptor; may be a partial agonist of a B-cell activating factor receptor; may be a competitive antagonist of a B-cell activating factor receptor; may be a non-competitive antagonist of a B-cell activating factor receptor; or any combination thereof. In some embodiments, immunotherapeutic agent or a salt thereof at least partially may prevent ligand binding to a transmembrane activator and calcium-modulator and cyclophilin ligand interactor receptor; may be a partial antagonist of a transmembrane activator and calcium-modulator and cyclophilin ligand interactor receptor; may be a partial agonist of a transmembrane activator and calcium-modulator and cyclophilin ligand interactor receptor; or any combination thereof. In some embodiments, an immunotherapeutic agent or a salt thereof at least partially may prevent ligand binding to a B-cell maturation antigen receptor; may be a partial antagonist of a B-cell maturation antigen receptor; may be a partial agonist of a B-cell maturation antigen receptor; or any combination thereof.

An immunotherapeutic agent may be a small molecule, an antibody, a partial antibody, or any combination thereof. An immunotherapeutic agent may be an antibody or a salt thereof. The immunotherapeutic agent may be a human antibody, a humanized monoclonal antibody, or a salt thereof.

In some embodiments, the immunotherapeutic agent or a salt thereof may be ardenermin, briobacept, blisibimod (A-623, CAS number 1236126-45-6), belimumab (Benlystra®, CAS number 356547-88-1), a transmembrane activator and calcium-modulator and cyclophilin ligand interactorcept, MEDI-0700, NOV-5, rGel/BLyS, tabalumab (CAS number 1143503-67-7), atacicept (CAS number 845264-92-8), RCT-18, a salt of any of these, and any combination thereof. An immunotherapeutic agent or a salt thereof may be selected from the group consisting of: MEDI-700, NOV-5, rGel/BLyS, a salt of any of these, and any combination thereof. The immunotherapeutic agent may be tabalumab (CAS number 1143503-67-7) or a salt thereof. The immunotherapeutic agent may be LY_2127399 or a salt thereof. The immunotherapeutic agent may be atacicept (CAS number 845264-92-8) or a salt thereof. The immunotherapeutic agent may be blisibimod (A-623, CAS number 1236126-45-6) or a salt thereof. The immunotherapeutic agent may be belimumab (Benlystra®, CAS number 356547-88-1) or a salt thereof. A BAFF/BAFF-R inhibitor may be combined with avelumab (Bavencio®, CAS number 1537032-82-8). A BAFF/BAFF-R inhibitor may be combined with pembrolizumab (Keytruda®, CAS number 1374853-91-4).

In some embodiments, a PD-L1 inhibitor may be avelumab (Bavencio®, CAS number 1537032-82-8). In some embodiments, a PD-L1 inhibitor may be Bavencio. In some embodiments, a PD-L1 inhibitor may be atezoluzumab. In some embodiments, a PD-L1 inhibitor may be tecentriq. In some embodiments, a PD-1 inhibitor may be pembrolizumab (Keytruda®, CAS number 1374853-91-4). In some embodiments, a PD-1 inhibitor may be keytruda. In some embodiments, a PD-1 inhibitor may be MK_3475. In some embodiments, a PD-1 inhibitor may be lambrolizumab. In some embodiments, a PD-1 inhibitor may be nivolumab (Opdivo®, CAS number 946414-94-4). In some embodiments, a PD-1 inhibitor may be opdivo. In some embodiments, a PD-1 inhibitor may be ONO_4538. In some embodiments, a PD-1 inhibitor may be BMS_936558. In some embodiments, a PD-1 inhibitor may be MDX_1106.

An immunotherapeutic agent may be an antibody. An immunotherapeutic agent may be a human monoclonal antibody. An immunotherapeutic agent may be a humanized monoclonal antibody, chimeric antibody, or an isolated or purified version of any of the above. An immunotherapeutic agent may be tabalumab (CAS number 1143503-67-7) or a fragment or a salt thereof Δny compound, molecule, protein, antibody, antibody fragment, or salt of any thereof, described herein may be isolated and purified.

An immunotherapeutic agent may be a checkpoint inhibitor, an immune agonist, a vaccine, a virus, or a T-cell.

Compositions may comprise a therapeutically effective amount of a pharmaceutical agent and an immunotherapeutic agent. Compositions may comprise a therapeutically effective amount of a pharmaceutical agent and an immunotherapeutic agent, wherein the immunotherapeutic agent may be tabalumab (CAS number 1143503-67-7) or a fragment or a salt thereof.

Compositions may comprise a therapeutically effective amount of a pharmaceutical agent and an immunotherapeutic agent, wherein the immunotherapeutic agent may be briobacept, rGel/BLyS, BLyS radiolabeled, Xencor, NOV-5, ardenermin, anti-BR3, blisibimod (A-623, CAS number 1236126-45-6), belimumab (Benlystra®, CAS number 356547-88-1), IL-17-bispecific antibody, anti-BLyS/APRIL antibody fusion protein, or a fragment or a salt thereof.

Compositions may comprise a therapeutically effective amount of a pharmaceutical agent and an immunotherapeutic agent, wherein the immunotherapeutic agent may be florbetapir, solanezumab, scyllo-inositol, gantenerumab, flutafuranol F18, cromolyn sodium+ibuprofen, AZTherapies, crenezumab, CAD-106, aducanumab, UB-311, GSK-933776A, BAN-2401, ACI-24, VM-100, SAR-228810, NGP-555, MRZ-99030, MEDI-1814, LY-3002813, Lu-AF-20513, HSRx 888+donepezil hydrochloride, Exebryl-1, bisnorcymserine, anti-amyloid beta antibody, Kyowa Hakko Kirin, ALZ-801, Affitope AD-03, AAB-003, (+)-phenserine, TRV-101, RV-03, P-8, NPT440-1, MRK-560, KAL-ABP, IN-NO1-OX2, IN-N01, CB-301, C12, Pharma Bio, CT-01344, BAN-2502, AZP-2006, anti-beta amyloid, Aerie, anti-amyloid beta programme, anti-amyloid beta antibody-2, anti-amyloid beta antibody, ALZT-Patch, ALZ-201, AGT-160, ADx, ACU-193, ACI-812, ACI-260, ZT-331, ACC-001, tramiprosate, SIB-1281, ponezumab, PF-4382923, GT3001, GT2501, GT2342, bapineuzumab, AN-1792, ACU-244, V-950, U-101033E, CHF-5022, SeV-2-0401, SEN-1576, SEN-1500, SEN-1269, SEN-1176, RV-02, RV-01, RS-0406, PX-106, PTI-L07665, PTI-48579, PTI-3001, PTI-2001, PTI-00703, protease nexin-1, SIBIA, phenserine(−)-eseroline phenylcarbamate(−)-phenserine, Pharmaprojects No. 5550, Pharmaprojects No. 4246, DP-74, NU-700, NP-0361, MPI-442690, MPI-423948MPI-127585, MK-3328, MER-5101, KMS-88009KMS-88016, INM-176, ESBA-212, EDN-OL1, DP-68, DLX-212, DBT-1339, Cymserine, N1-phenethylnorcymserine, CLR-01, CaprospinolSP-233, BAN-2203AD-2203, AZD-2995, AZD-2184, ARC-031, amyloid inhibiting peptides, amyloid beta modulator, AMDL receptor antagonist, ALZT-OP2, SPI-008SPI-0090, SPI-019, AGT-3100, AGT-100, Affitope AD-02, Affitope AD-01, ACU-CD0061, ACU-5A5, ACU-347, ACU-193, ABP-102, DRM-106, or a fragment or a salt of any of these.

A pharmaceutical agent may be an immunotherapeutic agent, a chemotherapeutic agent, a targeted therapeutic agent, a hormonal therapy agent, cell-based therapy agent, or radiation therapy agent.

In some embodiments, a composition may comprise a pharmaceutical agent and an immunotherapeutic agent, wherein the immunotherapeutic agent may be a B-cell activating factor inhibitor and at least partially may block the BAFF receptor cascade.

Pharmaceutical compositions may be formulated using one or more physiologically acceptable carriers including excipients and auxiliaries that may facilitate processing of the pharmaceutical composition into preparations that may be used pharmaceutically. Proper formulation may be dependent upon the route of administration chosen. A summary of pharmaceutical compositions may be found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

The compositions and methods of the present disclosure may be utilized to treat an individual in need thereof. The individual may be a mammal such as a human, or a non-human mammal. A subject may be a human. The human may be an adult male, an adult female, a female child, or a male child. When administered to an animal, such as a human, the composition or the pharmaceutical composition, may be administered as a pharmaceutical composition comprising, for example, a therapeutically effective amount of an anti-programmed cell death protein 1 agent and an immunotherapeutic agent and a pharmaceutically acceptable carrier or excipient. Pharmaceutically acceptable carriers may include, for example, aqueous solutions such as water or physiologically buffered saline or other solvents or vehicles such as glycols, glycerol, oils such as olive oil, or injectable organic esters. In an embodiment, when such pharmaceutical compositions may be for human administration, particularly for invasive routes of administration, e.g., routes, such as injection or implantation, that circumvent transport or diffusion through an epithelial barrier, the aqueous solution may be pyrogen-free, or substantially pyrogen-free. The excipients may be chosen, for example, to affect delayed release of an agent or to selectively target one or more cells, tissues or organs. The pharmaceutical composition may be in dosage unit form such as tablet, capsule, granule, lyophile for reconstitution, powder, solution, syrup, suppository, injection or the like. The composition may also be present in a transdermal delivery system, e.g., a skin patch. The composition may also be present in a solution suitable for topical administration, such as an eye drop.

A pharmaceutically acceptable excipient may contain physiologically acceptable agents that act, for example, to stabilize, increase solubility or to increase the absorption of a composition such as a therapeutically effective amount of an anti-programmed cell death protein 1 agent and an immunotherapeutic agent. Such physiologically acceptable agents can include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, may depend, for example, on the route of administration of the composition. The preparation or pharmaceutical composition may be a self-emulsifying drug delivery system or a self-micro emulsifying drug delivery system. The pharmaceutical composition (preparation) also may be a liposome or other polymer matrix, which may have incorporated therein, for example, a therapeutically effective amount of an anti-programmed cell death protein 1 agent and an immunotherapeutic agent. Liposomes, for example, which may comprise phospholipids or other lipids, may be nontoxic, physiologically acceptable and metabolizable carriers that may be relatively simple to make and administer.

In some embodiments, the immunotherapeutic agent or a salt thereof may be administered in a pharmaceutical composition.

In one aspect, the current disclosure may provide a composition comprising an anti-programmed cell death protein 1 agent, an anti-programmed death ligand 1 agent, an anti-programmed death ligand 2 agent, or a salt of any one thereof, or any combination thereof, and an immunotherapeutic agent or a salt thereof.

In some embodiments, the composition may be a pharmaceutical composition. The pharmaceutical composition may be in unit dose form. The immunotherapeutic agent or a salt thereof may be an antibody or a salt thereof. The immunotherapeutic agent or a salt thereof may be a human or humanized monoclonal antibody or a salt thereof. The immunotherapeutic agent or a salt thereof may be selected from the group consisting of: ardenermin, briobacept, blisibimod (A-623, CAS number 1236126-45-6), belimumab (Benlystra®, CAS number 356547-88-1), a transmembrane activator and calcium-modulator and cyclophilin ligand interactorcept, MEDI-0700, NOV-5, rGel/BLyS, tabalumab (CAS number 1143503-67-7), atacicept (CAS number 845264-92-8), RCT-18, a salt of any of these, and any combination thereof. The immunotherapeutic agent or a salt thereof may be tabalumab (CAS number 1143503-67-7) or a salt thereof. A composition may further comprise a pharmaceutically acceptable excipient.

In some embodiments, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof may be independently administered in an amount from about 0.1 mg to about 10 g. In some embodiments, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof may be independently from about 0.001% to about 99% by weight of the composition. In some embodiments, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof may be independently administered in an amount of from about 0.1 mg to about 100 mg per kg body weight.

In some embodiments, the composition may be in a form of a tablet, a capsule, a gel, or a liquid formulation.

In one aspect, the present disclosure provides for a kit comprising a composition of any one of the preceding claims. In some embodiments, the kit may contain instructions for use.

In one aspect, the present disclosure provides a method of making a kit disclosed herein. The method may comprise contacting or combining the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof. In some embodiments, the composition may be in a form of a tablet, a capsule, a gel, or a liquid formulation.

In one aspect, the present disclosure may provide a method of making a composition of any one of the preceding claims. the method may comprise contacting or combining the anti-programmed cell death protein 1 agent, the anti-programmed death ligand 1 agent, the anti-programmed death ligand 2 agent, or a salt thereof or any combination thereof, and the immunotherapeutic agent or a salt thereof.

### Routes of Administration

A pharmaceutical composition may be administered to a subject by any of a number of routes of administration including, for example, orally, for example, drenches as in aqueous or non-aqueous solutions or suspensions, tablets, capsules, including sprinkle capsules and gelatin capsules, boluses, powders, granules, pastes for application to the tongue; absorption through the oral mucosa, e.g., sublingually; anally, rectally or vaginally, for example, as a pessary, cream or foam; parenterally, including intramuscularly, intravenously, subcutaneously or intrathecal as, for example, a sterile solution or suspension; nasally; intraperitoneally; subcutaneously; transdermal, for example, as a patch applied to the skin; and topically, for example, as a cream, ointment or spray applied to the skin, as an eye drop, or any combination thereof. The pharmaceutical composition may also be formulated for inhalation. A therapeutically effective amount of an anti-programmed cell death protein 1 agent and an immunotherapeutic agent may be simply dissolved or suspended in sterile water. A composition may be administered directly to a cancer or a tumor. Administration of a composition may be to a different location of the body than where the cancer or tumor may be detected or present.

A pharmaceutical composition may be a sterile aqueous or non-aqueous solution, suspension or emulsion, e.g., a microemulsion. The excipients described herein may be examples and are in no way limiting. An effective amount or therapeutically effective amount may refer to an amount of the one or more pharmaceutical compositions administered to a subject, either as a single dose or as part of a series of doses, which is effective to produce a desired therapeutic effect.

Subjects may generally be monitored for therapeutic effectiveness using assays and diagnostic methods suitable for the condition being treated. Pharmacokinetics of a therapeutically effective amount of an anti-programmed cell death protein 1 agent and an immunotherapeutic agent, or one or more metabolites thereof, that may be administered to a subject may be monitored by determining the level of the pharmaceutical composition or metabolite in a biological fluid, for example, in the blood, blood fraction, e.g., serum, and/or in the urine, and/or other biological sample or biological tissue from the subject.

The dose of a therapeutically effective amount of an anti-programmed cell death protein 1 agent and an immunotherapeutic agent described herein for treating a cancer may depend upon the subject's condition, that is, stage of the disease, severity of symptoms caused by the disease, general health status, as well as age, gender, and weight, and other factors apparent to a person skilled in the medical art. A subject may have been previously diagnosed with a cancer or a tumor. A subject may have previously undergone treatment for a cancer or a tumor. A subject may be currently undergoing treatment for a cancer or a tumor. The current treatment of the subject may be less than therapeutically effective. The subject may have been previously treated with a checkpoint inhibitor, and the cancer or the tumor may have been at least partially refractive. The cancer or tumor may be considered partially refractive if the mass or volume of the cancer or tumor continues to grow at a constant rate, or if the mass or volume of the cancer or tumor may not substantially change or decrease.

Pharmaceutical compositions may be administered in a manner appropriate to the disease to be treated as determined by persons skilled in the medical arts. In addition to the factors described herein and above related to use of pharmaceutical composition for treating a cancer, suitable duration and frequency of administration of the pharmaceutical composition may also be determined or adjusted by such factors such as the condition of the subject, the type and severity of the subject's disease, the particular form of the active ingredient, and the method of administration. Optimal doses of an agent may generally be determined using experimental models and/or clinical trials. The optimal dose may depend upon the body mass, weight, or blood volume of the subject. Design and execution of pre-clinical and clinical studies for a pharmaceutical composition, including when administered for prophylactic benefit, described herein may be within the skill of a person skilled in the relevant art.

When two or more pharmaceutical compositions may be administered to treat a cancer, the optimal dose of each pharmaceutical composition may be different, such as less than when either agent may be administered alone as a single agent therapy. Two pharmaceutical compositions in combination may act synergistically, and either agent may be used in a lesser amount than if administered alone. Administration of a checkpoint inhibitor and an immunotherapeutic agent may act synergistically or more than additively, when compared to either agent administered alone. A composition comprising a checkpoint inhibitor and an immunotherapeutic agent may produce a more than additive effect on a tumor or a cancer. The synergy of a combination of the current disclosure may be at least about: 1%, 2%, 3%, 5%, 10%, 20%, 30%, 40%, 50%, 60%, 70%, 80%, 90%, or more greater than the additive effect.

In some embodiments, a composition of the disclosure comprising an anti-programmed cell death protein 1 agent and an immunotherapeutic agent may be more effective as compared to the corresponding additive effect of the anti-programmed cell death protein 1 agent and an immunotherapeutic agent. A composition of the disclosure may be at least about: 15% more effective, 20% more effective, 25% more effective, 30% more effective, 35% more effective, 40% more effective, 45% more effective, 50% more effective, 55% more effective, 60% more effective, 65% more effective, 70% more effective, 75% more effective, 80% more effective, 85% more effective, 95% more effective, or 100% more effective than the effect of a monotherapy comprising an anti-programmed cell death protein 1 agent, and/or the effect of a monotherapy comprising an immunotherapeutic agent.

A composition of the disclosure may be at least about: 1% more effective, 5% more effective, 10% more effective, 15% more effective, 20% more effective, 25% more effective, 30% more effective, 35% more effective, 40% more effective, 45% more effective, 50% more effective, 55% more effective, 60% more effective, 65% more effective, 70% more effective, 75% more effective, 80% more effective, 85% more effective, 95% more effective, or 100% more effective than the corresponding additive effect of the anti-programmed cell death protein 1 agent and an immunotherapeutic agent.

An amount of a pharmaceutical composition or active therein that may be administered per day may be, for example, from, between about 0.01 mg/kg and about 100 mg/kg, e.g., from between about 0.1 to about 1 mg/kg, from between about 1 to about 10 mg/kg, from between about 10 mg/kg and about 50 mg/kg, from between about 50 mg/kg to about 100 mg/kg body weight. In other embodiments, the amount of a pharmaceutical composition that may be administered per day may be from between about 0.01 mg/kg and about 1000 mg/kg, from between about 100 mg/kg and about 500 mg/kg, or from between about 500 mg/kg and about 1000 mg/kg body weight. The optimal dose, per day or per course of treatment, may be different for the cancer or tumor to be treated and may also vary with the administrative route and therapeutic regimen.

Pharmaceutical compositions comprising a pharmaceutical composition may be formulated in a manner appropriate for the delivery method by using techniques routinely practiced in the art. The composition may be in the form of a solid, e.g., tablet, capsule, semi-solid, e.g., gel, liquid, or gas, e.g., aerosol. The pharmaceutical composition may be administered as a bolus infusion.

Exemplary pharmaceutically acceptable excipients may include sterile saline and phosphate buffered saline at physiological pH. Preservatives, stabilizers, dyes, buffers, and the like may be provided in the pharmaceutical composition. In addition, antioxidants and suspending agents may also be used. The type of excipient selected may be based on the mode of administration, as well as the chemical composition of the pharmaceutical composition. Alternatively, compositions described herein may be formulated as a lyophilizate. A composition described herein may be lyophilized or otherwise formulated as a lyophilized product using one or more appropriate excipient solutions for solubilizing and/or diluting the pharmaceutical composition of the composition upon administration. In other embodiments, the pharmaceutical composition may be encapsulated within liposomes. Pharmaceutical compositions may be formulated for any appropriate manner of administration described herein and in the art.

A pharmaceutical composition, which may be combined with at least one pharmaceutically acceptable excipient to form a pharmaceutical composition, is administered directly to the target tissue or organ comprising tumor cells that contribute to manifestation of the disease or disorder.

A pharmaceutical composition, e.g., for oral administration or for injection, infusion, subcutaneous delivery, intramuscular delivery, intraperitoneal delivery or other method, may be in the form of a liquid. A liquid pharmaceutical composition may include, for example, one or more of the following: a sterile diluent such as water, saline solution, physiological saline solution, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition may be enclosed in ampoules, disposable syringes or multiple dose vials made of glass or plastic. The use of physiological saline may be used, and an injectable pharmaceutical composition may be sterile. In another embodiment, for treatment of an ophthalmological condition or disease, a liquid pharmaceutical composition may be applied to the eye in the form of eye drops. A liquid pharmaceutical composition may be delivered orally.

For oral formulations, at least one of the pharmaceutical compositions described herein may be used alone or in combination with appropriate additives to make tablets, powders, granules or capsules, and if desired, with diluents, buffering agents, moistening agents, preservatives, coloring agents, and flavoring agents. The pharmaceutical compositions may be formulated with a buffering agent to provide for protection of the pharmaceutical composition from low pH of the gastric environment and/or an enteric coating. A pharmaceutical composition included in a pharmaceutical composition may be formulated for oral delivery with a flavoring agent, e.g., in a liquid, solid or semi-solid formulation and/or with an enteric coating.

A pharmaceutical composition comprising any one of the pharmaceutical compositions described herein may be formulated for sustained release, slow release, timed release, or controlled release. Such compositions may generally be prepared and administered by, for example, oral, rectal, intradermal, or subcutaneous implantation, or by implantation at the desired target site. Sustained-release formulations may contain the pharmaceutical composition dispersed in a carrier matrix and/or contained within a reservoir surrounded by a rate controlling membrane. Excipients for use within such formulations may be biocompatible, and may also be biodegradable. The amount of pharmaceutical composition contained within a sustained release formulation depends upon the site of implantation, the rate and expected duration of release, and the nature of the condition, disease or disorder to be treated or prevented.

The pharmaceutical compositions comprising a pharmaceutical composition may be formulated for transdermal, intradermal, or topical administration. The compositions may be administered using a syringe, bandage, transdermal patch, insert, or syringe-like applicator, as a powder/talc or other solid, liquid, spray, aerosol, ointment, foam, cream, gel, paste. The active compositions may also be delivered via iontophoresis. Preservatives may be used to prevent the growth of fungi and other microorganisms. Suitable preservatives may include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, or any combination thereof.

Pharmaceutical compositions may be formulated as emulsions for topical application. The pharmaceutical composition described herein may be formulated as in inhalant. Inhaled methods may deliver medication directly to the airway. The pharmaceutical composition may be formulated with oleaginous bases or ointments to form a semisolid composition with a desired shape.

Controlled or sustained release transdermal or topical formulations may be achieved by the addition of time-release additives, such as polymeric structures, and matrices. For example, the compositions may be administered through use of hot-melt extrusion articles, such as bioadhesive hot-melt extruded film. The formulation may comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the pharmaceutical composition.

A polymer formulation may also be utilized to provide controlled or sustained release. Bioadhesive polymers may be used. By way of example, a sustained-release gel and the pharmaceutical composition may be incorporated in a polymeric matrix, such as a hydrophobic polymer matrix. Examples of a polymeric matrix may include a microparticle. The microparticles may be microspheres, and the core may be of a different material than the polymeric shell. Alternatively, the polymer may be cast as a thin slab or film, a powder produced by grinding or other standard techniques, or a gel such as a hydrogel. The polymer may also be in the form of a coating or part of a bandage, stent, catheter, vascular graft, or other device to facilitate delivery of the pharmaceutical composition. The matrices may be formed by solvent evaporation, spray drying, or solvent extraction.

Kits with unit doses of one or more of the agents described herein, usually in oral or injectable doses, may be provided. Such kits may include a container containing the unit dose, an informational package insert describing the use and attendant benefits of the drugs in treating a tumor or cancer, instructions for use, and optionally an appliance or device for delivery of the composition.

### Dosing and Treatment Regimens

The pharmaceutical compositions described herein may be used in the preparation of medicaments for the prevention or treatment of a cancer. In addition, a method for treating any of the diseases or conditions described herein in a subject in need of such treatment, involves administration of pharmaceutical compositions containing at least a therapeutically effective amount of an anti-programmed cell death protein 1 agent and an immunotherapeutic agent, or a pharmaceutically acceptable salt, pharmaceutically acceptable prodrug, or pharmaceutically acceptable solvate thereof, in therapeutically effective amounts to the subject.

In some embodiments, the present disclosure may provide a method for treating any of the diseases or conditions described herein in a subject in need of such treatment, wherein the treatment may involve administration of pharmaceutical composition containing at least a therapeutically effective amount of an anti-programmed cell death protein 1 agent and an immunotherapeutic agent, wherein the immunotherapeutic agent may be tabalumab (CAS number 1143503-67-7) or a fragment or a salt thereof.

A count of B cells, or a component thereof, or plasma cells, or a component thereof, may be observed in a sample of whole blood, blood serum, blood plasma, tumor, saliva, sweat, or urine of a subject. A specific treatment method for each individual subject may be determined based on a count of B cells or plasma cells or T cells, or any combination thereof.

In some embodiments, the present disclosure may provide a method of administering an anti-programmed cell death protein 1 agent or a salt thereof or an anti-PD-L1 agent or a salt thereof if a count of T cells in a cancer or a tumor sample of a subject is greater than about: 10 cells/mm², 100 cells/mm², 200 cells/mm², 300 cells/mm², 400 cells/mm², 500 cells/mm², 600 cells/mm², 700 cells/mm², 800 cells/mm², 900 cells/mm², or 1000 cells/mm², and a count of B cells is less than 1000 cells/mm², 900 cells/mm², 800 cells/mm², 700 cells/mm², 600 cells/mm² 500 cells/mm², 400 cells/mm², 300 cells/mm², 200 cells/mm², 150 cells/mm², 100 cells/mm², 90 cells/mm², or 75 cells/mm².

In some embodiments, the present disclosure may provide a method of administering an anti-programmed cell death protein 1 agent or a salt thereof or an anti-PD-L1 agent or a salt thereof, and an immunotherapeutic agent at a salt thereof if a count of T cells in a cancer or a tumor sample of a subject may be greater than about: 10 cells/mm², 100 cells/mm², 200 cells/mm², 300 cells/mm², 400 cells/mm², 500 cells/mm², 600 cells/mm², 700 cells/mm², 800 cells/mm², 900 cells/mm², or 1000 cells/mm² and a count of B cells, a count of plasma cells, or a combination of B cells and plasma cells may be greater than about: 10 cells/mm², 100 cells/mm², 200 cells/mm², 300 cells/mm², 400 cells/mm², 500 cells/mm², 600 cells/mm², 700 cells/mm² 800 cells/mm², 900 cells/mm², or 1000 cells/mm² in a cancer or a tumor sample of a subject.

In some embodiments, the present disclosure may provide a method of administering an anti-programmed cell death protein 1 agent or a salt thereof or an anti-PD-L1 agent or a salt thereof if a percentage of T cells in a cancer sample or a tumor sample of a subject may be greater than about: 5%, 10%, 15%, 20%, 30%, 40%, 50%, 60%, 70%, 80%, 90%, or more, and a percentage of B cells may be less than about: 50%, 40%, 30%, 20%, 15%, 10%, 5%, or less.

In some embodiments, the present disclosure may provide a method of administering an anti-programmed cell death protein 1 agent or a salt thereof or an anti-PD-L1 agent or a salt thereof, and an immunotherapeutic agent or a salt thereof if a percentage of T cells in a cancer or a tumor sample of a subject may be less than about: 50%, 40%, 30%, 20%, 15%, 10%, 5%, or less, and a percentage of B cells, a percentage of plasma cells, or a combination of a percentage of B cells and a percentage of plasma cells may be greater than about: 5%, 10%, 15%, 20%, 30%, 40%, 50%, 60%, 70%, 80%, or 90% in a cancer sample of a subject.

The compositions containing the pharmaceutical compositions described herein may be administered for prophylactic and/or therapeutic treatments. In therapeutic applications, the compositions may be administered to a subject already suffering from a disease or condition, in an amount sufficient to cure or at least partially arrest the symptoms of the disease or condition. Amounts effective for this use may depend on the severity and course of the disease or condition, previous therapy, the subject's health status, weight, and response to the drugs, and the judgment of the treating physician.

In the case wherein the subject's condition does not improve, upon the doctor's discretion the administration of the pharmaceutical composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the administration of the pharmaceutical composition may be given continuously; alternatively, the dose of drug being administered may be temporarily reduced or temporarily suspended for a length of time (i.e., a “drug holiday”). The length of the drug holiday may vary from between about 2 days and about 1 year, including by way of example only about: 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from about 10% to about 100%, including, by way of example, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% relative to a dosage administered before or after the drug holiday.

In some cases, the administration of the pharmaceutical composition may be given indefinitely.

In the cases, a subject may exhibit a desired response to an anti-programmed cell death protein 1 therapy. In some cases, a subject may exhibit a partial desired response to an anti-programmed cell death protein 1 therapy. In some cases, a subject may have been previously treated with a monotherapy. In some cases, a subject may become resistant to a monotherapy. A subject may become resistant, or show signs of resistance, after about: 1 day, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, 400 days, 500 days, 600 days, 700 days, 800 days, 900 days, or more after treatment of a monotherapy.

In some embodiments, the current disclosure provides a method of treating a patient in need thereof comprising a treatment regimen of a monotherapy for a desired amount of time, followed by treatment of a combination therapy for a desired amount of time.

Once improvement of the subject's conditions may have occurred, a maintenance dose may be administered if necessary. Subsequently, the dosage or the frequency of administration, or both, may be reduced, as a function of the symptoms, to a level at which the improved disease, disorder or condition is retained. Subjects can, however, require intermittent treatment on a long-term basis upon any recurrence of symptoms.

The amount of a given agent that may correspond to such an amount may vary depending upon factors such as the particular pharmaceutical composition, disease or condition and its severity, the identity (e.g., weight) of the subject or host in need of treatment, but may nevertheless be determined in a manner recognized in the field according to the particular circumstances surrounding the case, including, e.g., the specific agent being administered, the route of administration, the condition being treated, and the subject or host being treated. In general, however, doses employed for adult human treatment may typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation may be divided into unit doses containing appropriate quantities of one or more pharmaceutical compositions. The unit dosage may be in the form of a package containing discrete quantities of the formulation. Non-limiting examples may be packaged tablets or capsules, and powders in vials or ampoules. Aqueous suspension compositions may be packaged in single-dose non-reclosable containers. Alternatively, multiple-dose reclosable containers may be used, in which case it may be typical to include a preservative in the composition. By way of example only, formulations for parenteral injection may be presented in unit dosage form, which include, but may be not limited to ampoules, or in multi-dose containers, with an added preservative.

The single dosages appropriate for the pharmaceutical compositions described herein may be from about 0.01 mg/kg to about 20 mg/kg. In one embodiment, the single dosage of a checkpoint inhibitor may be from about 0.1 mg/kg to about 10 mg/kg. In one embodiment, the single dosage of an anti-programmed cell death protein 1 agent, an anti-programmed death ligand 1 agent, an anti-programmed death ligand 2 agent, a salt of any one thereof, or any combination thereof, may be from about 0.1 mg/kg to about 10 mg/kg. An indicated single dosage in the larger mammal, including, but not limited to, humans, may be in the range from about 0.5 mg to about 1000 mg, conveniently administered in a single dose or in divided doses, including, but not limited to, up to four times a day or in extended release form. Suitable unit dosage forms for administration may include from about 1 to about 500 mg active ingredient. In one embodiment, the unit dosage may be about 1 mg, about 5 mg, about, 10 mg, about 20 mg, about 50 mg, about 100 mg, about 200 mg, about 250 mg, about 400 mg, or about 500 mg. In some embodiments, the an anti-programmed cell death protein 1 agent, an anti-programmed death ligand 1 agent, an anti-programmed death ligand 2 agent, a salt of any one thereof, or any combination thereof, may be 0.001% to 99% by weight of a pharmaceutical composition.

In one embodiment, a single dose of an immunotherapeutic agent may be from about 0.1 mg/kg to about 10 mg/kg. In one embodiment, a single dose of blisibimod (A-623, CAS number 1236126-45-6), belimumab (Benlystra®, CAS number 356547-88-1), tabalumab (CAS number 1143503-67-7), atacicept (CAS number 845264-92-8), or a salt of any one thereof, may be from about 0.1 mg/kg to about 10 mg/kg.

The foregoing ranges may be merely suggestive, as the number of variables in regard to an individual treatment regime may be large, and considerable excursions from these recommended values may be not uncommon. Such dosages may be altered depending on a number of variables, not limited to the activity of the pharmaceutical composition used, the disease or condition to be treated, the mode of administration, the requirements of the individual subject, the severity of the disease or condition being treated, and the judgment of the practitioner.

Toxicity and therapeutic efficacy of such therapeutic regimens may be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects may be the therapeutic index and it may be expressed as the ratio between LD₅₀ and ED₅₀. The data obtained from cell culture assays and animal studies may be used in formulating a range of dosage for use in human. The dosage may vary within this range depending upon the dosage form employed and the route of administration utilized.

The method used for cancer therapy with a pharmaceutical composition described herein may comprise one or more of a decreased single dose, decreased cumulative dose over a single therapeutic cycle, or decreased cumulative dose of the pharmaceutical composition over multiple therapeutic cycles compared with the amount required for cancer therapy.

The treatment regimen of the methods for cancer therapy comprises administering a pharmaceutical composition for a time sufficient and in an amount sufficient that kills tumor cells. The pharmaceutical composition may be administered within a treatment cycle, which treatment cycle may comprise a treatment course followed by a non-treatment interval. A treatment course of administration refers herein to a finite time frame over which one or more doses of the pharmaceutical composition on one or more days may be administered. The finite time frame may be also called herein a treatment window.

Ingredients of a pharmaceutical composition described herein may be administered concurrently (e.g., simultaneously, essentially simultaneously or within the same treatment protocol) or sequentially. An immunotherapeutic agent and a checkpoint inhibitor may be administered concurrently. An immunotherapeutic agent and a checkpoint inhibitor may be administered sequentially. An immunotherapeutic agent and a checkpoint inhibitor may be administered within the same formulation. An immunotherapeutic agent and a checkpoint inhibitor may be administered in different formulations within a single treatment schedule, or the immunotherapeutic agent and a checkpoint inhibitor may be administered on different treatment schedules.

In some embodiments, a composition of the current disclosure may be made by contacting combining, mixing, adding, or any combination thereof, an anti-PD-1, an anti-PD-L1, an anti-PD-L2, or a salt of any one thereof, or any combination thereof, and an immunotherapeutic agent or a salt thereof. A kit of the current disclosure may be made by contacting combining, mixing, adding, or any combination thereof, an anti-PD-1, an anti-PD-L1, an anti-PD-L2, or a salt of any one thereof, or any combination thereof, and an immunotherapeutic agent or a salt thereof.

A method of the current disclosure may comprise administering the pharmaceutical composition in at least two treatment cycles. In a specific embodiment, the non-treatment interval may be at least about 2 weeks or between from at least about 0.5 to about 12 months, such as at least about one month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months (i.e., 1 year). The non-treatment interval may be between about 1 years to about 2 years or between about 1 years to about 3 years, or longer. Each treatment course may be no longer than about 1 month, no longer than about 2 months, or no longer than about 3 months; or is no longer than about: 1, 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days.

The treatment window (i.e., treatment course) may be only one day. A single treatment course may occur over no longer than about: 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days. During such treatment windows, the pharmaceutical composition may be administered at least on about two days (i.e., two days or more) with a variable number of days on which the agent may be not administered between the at least two days of administration. Stated another way, within a treatment course when the pharmaceutical composition may be administered on two or more days, the treatment course may have one or more intervals of one or more days when the pharmaceutical composition, is not administered. By way of non-limiting example, when the pharmaceutical composition may be administered on 2 or more days during a treatment course not to exceed 21 days, the agent may be administered on any total number of days between from about: 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days. The pharmaceutical composition may be administered to a subject during a treatment course of 3 days or more, and the agent may be administered every 2^{nd} day (i.e., every other day). When the pharmaceutical composition may be administered to a subject for a treatment window of about 4 days or more, the pharmaceutical composition may be administered every about 3^{rd} day. In one embodiment, the pharmaceutical composition may be administered on at least about two days during a treatment course that is at least about 2 days and no more than about 21 days (i.e., from about 2 days to about 21 days); at least about 2 days and no longer than about 14 days (i.e., from about 2 days to about 14 days); at least about 2 days and no longer than about 10 days (i.e., from about 2 days to about 10 days); or at least about 2 days and no longer than about 9 days (i.e., from about 2 days to about 9 days); or at least about 2 days and no longer than about 8 days (i.e., from about 2 days to about 8 days). In other specific embodiments, the pharmaceutical composition may be administered on at least about two days (i.e., about 2 days or more) during a treatment window may be at least about 2 days and no longer than about 7 days (i.e., from about 2 days to about 7 days); at least about 2 days and no longer than about 6 days (i.e., from about 2 days to about 6 days) or at least about 2 days and no more than about 5 days (i.e., from about 2 days to about 5 days) or at least about 2 days and no longer than about 4 days (i.e., from about 2 days to about 4 days). In yet another embodiment, the treatment window is at least about 2 days and no longer than about 3 days (i.e., from about 2 days to about 3 days), or about 2 days. The treatment course may be no longer than about 3 days. The treatment course may be no longer than about 5 days. The treatment course may be no longer than about: 7 days, 10 days, or 14 days or 21 days. The pharmaceutical composition may be administered on at least about two days (i.e., about 2 or more days) during a treatment window that may be at least about 2 days and no longer than about 11 days (i.e., from about 2 days to about 11 days); or the pharmaceutical composition may be administered on at least about two days (i.e., about 2 or more days) during a treatment window that is at least about 2 days and no longer than about 12 days (i.e., from about 2 days to about 12 days); or the pharmaceutical composition may be administered on at least about two days (i.e., about 2 or more days) during a treatment window that may be at least about 2 days and no more than about 13 days (i.e., from about 2 days to about 13 days); or the pharmaceutical composition may be administered on at least about two days (i.e., about 2 or more days) during a treatment course that may be at least about 2 days and no more than about 15 days (i.e., from about 2 days to about 15 days); or the pharmaceutical composition may be administered on at least about two days (i.e., about 2 or more days) during a treatment course that may be at least about 2 days and no longer than about: 16 days, 17 days, 18 days, 19 days, or 20 days (i.e., 2-16, 2-17, 2-18, 2-19, 2-20 days, respectively). In other embodiments, the pharmaceutical composition may be administered on at least 3 days over a treatment course of at least about 3 days and no longer than any number of days between about 3 days and about 21 days; or may be administered on at least about 4 days over a treatment course of at least about 4 days and no longer than any number of days between about 4 days and about 21 days; or may be administered on at least about 5 days over a treatment course of at least about 5 days and no longer than any number of days between about 5 days and about 21 days; or may be administered on at least about 6 days over a treatment course of at least about 6 days and no longer than any number of days between about 6 and about 21 days; or may be administered at least about 7 days over a treatment course of at least about 7 days and no longer than any number of days between about 7 days and about 21 days; or may be administered at least about 8 days or about 9 days over a treatment course of at least about 8 days or about 9 days, respectively, and no longer than any number of days between about 8 days or about 9 days, respectively, and about 21 days; or may be administered at least about 10 days over a treatment course of at least about 10 days and no longer than any number of days between about 10 days and about 21 days; or is administered at least about 14 days over a treatment course of at least about 14 days and no longer than any number of days between about 14 days and about 21 days; or may be administered at least about 11 days or about 12 days over a treatment course of at least about 11 days or about 12 days, respectively, and no longer than any number of days between about 11 days or about 12 days, respectively, and about 21 days; or may be administered at least about 15 days or about 16 days over a treatment course of at least about 15 days or about 16 days, respectively, and no longer than any number of days between about 15 days or about 16 days, respectively, and about 21 days. By way of additional example, when the treatment course may be no longer than about 14 days, a pharmaceutical composition may be administered on at least about: 2, 3, 4, 5, 6, 7, 8, 9, 10, 11, 12, 13, and 14 days over a treatment of window of at least about: 2, 3, 4, 5, 6, 7, 8, 9, 10, 11, 12, 13, and 14 days, respectively, and no longer than about 14 days. When the treatment course may be no longer than about 10 days, a pharmaceutical composition may be administered on at least about: 2, 3, 4, 5, 6, 7, 8, 9, or 10 days over a treatment of window of at least about: 2, 3, 4, 5, 6, 7, 8, 9, or 10 days, respectively, and no longer than about 10 days. Similarly, when the treatment course may be no longer than about 7 days, a pharmaceutical composition may be administered on at least about 2, 3, 4, 5, 6, or 7 days over a treatment window of at least about 2, 3, 4, 5, 6, or 7 days, respectively, and no longer than about 7 days. When the treatment course may be no longer than about 5 days, a pharmaceutical composition may be administered on at least about 2, 3, 4, or 5 days over a treatment of window of at least about 2, 3, 4, or 5 days, respectively, and no longer than about 5 days.

With respect to a treatment course of about three or more days, doses of the pharmaceutical composition may be administered for a lesser number of days than the total number of days within the particular treatment window. By way of non-limiting example, when a course of treatment may have a treatment course of no more than about: 7, 10, 14, or 21 days, the number of days on which the pharmaceutical composition may be administered may be any number of days between from about 2 days and about: 7, 10, 14, or 21 days, respectively, and at any interval appropriate for the particular disease being treated, the pharmaceutical composition being administered, the health status of the subject and other relevant factors, which are discussed in greater detail herein. A person may appreciate that when the pharmaceutical composition may be administered on about two or more days over a treatment window, the agent may be delivered on the minimum number days of the window, the maximum number of days of the window, or on any number of days between the minimum and the maximum.

A treatment course may be one day or the treatment course may be of a length not to exceed about: 2, 3, 4, 5, 6, 7, 8, 9, 10, 11, 12, 13, or 14 days, which may be examples of a course wherein the pharmaceutical composition may be administered on two or more days over a treatment course not to exceed (i.e., no longer than) about: 2, 3, 4, 5, 6, 7, 8, 9, 10, 11, 12, 13, or 14 days, respectively. The treatment course may be about 2 weeks (about 14 days or 0.5 months), about 3 weeks (about 21 days), about 4 weeks (about one month), about 5 weeks, about 6 weeks (about 1.5 months), about 2 months (or about 60 days), or about 3 months (or about 90 days). A treatment course may be a single daily dosing of the pharmaceutical composition. In other embodiments, with respect to any treatment course a daily dose of the pharmaceutical composition may be as a single administration or the dose may be divided into 2, 3, 4, or 5 separate administrations to provide the total daily dose of the agent.

As described herein, within a treatment window when the pharmaceutical composition may be administered on two or more days, the treatment course may have one or more intervals of one or more days when the pharmaceutical composition, may be not administered. Solely as a non-limiting example, when a treatment window may be between two and seven days, a first dose may be administered on the first day of the treatment window and a second dose may be administered on the third day of the course, and a third dose may be administered on the seventh day of the treatment window. A person may appreciate that varying dosing schedules may be used during a particular treatment window. In other specific embodiments, the pharmaceutical composition may be administered daily on each consecutive day for the duration of the treatment course. A daily dose may be administered as a single dose or the daily dose may be divided into 2, 3, or 4, or 5 separate administrations to provide the total daily dose of the pharmaceutical composition.

The treatment course comprises a length of time during which the pharmaceutical composition may be administered daily. The pharmaceutical composition may be administered daily for 2 days. The pharmaceutical composition may be administered daily for 3 days. The pharmaceutical composition may be administered daily for 4 days. The pharmaceutical composition may be administered daily for 5 days. The pharmaceutical composition may be administered daily for 6 days. The pharmaceutical composition may be administered daily for 7 days. The pharmaceutical composition may be administered daily for 8 days. The pharmaceutical composition may be administered daily for 9 days. The pharmaceutical composition may be administered daily for 10 days. The pharmaceutical composition may be administered daily for 11 days. The pharmaceutical composition may be administered daily for 12 days. The pharmaceutical composition may be administered daily for 13 days. The pharmaceutical composition may be administered daily for 14 days. The treatment window (i.e., course) for each of the above examples may be no longer than about: 2, 3, 4, 5, 6, 7, 8, 9, 10, 11, 12, 13, 14 days, respectively.

The pharmaceutical composition may be administered every 2^{nd} day (i.e., every other day) for 3, 4, 5, 6, 7, 8, 9, 10, 11, 12, 13, or 14 days. The pharmaceutical composition may be administered every 3^{nd} day (i.e., one day receiving the agent followed by two days without receiving the agent) for 4, 5, 6, 7, 8, 9, 10, 11, 12, 13, or 14 days. The pharmaceutical composition may be administered on every 2^{nd}-3^{rd} day during a treatment window of 3, 4, 5, 6, 7, 8, 9, 10, 11, 12, 13, or 14 days. The pharmaceutical composition may be administered every 4^{th} day during a treatment course of 5, 6, 7, 8, 9, 10, 11, 12, 13, or 14 days; or every 5^{th} day during a treatment course of 6, 7, 8, 9, 10, 11, 12, 13, or 14 days. A person may appreciate the minimum numbers of days in a treatment window when the pharmaceutical composition may be administered every 6^{th}, 7^{th}, etc. day over a treatment window of a finite number of days as described herein.

A pharmaceutical composition may be administered daily for a longer duration than 14 days and may be administered at least about: 15, 16, 17, 18, 19, 20, or at least 21 days. The pharmaceutical composition may be administered daily on each of the about: 15, 16, 17, 18, 19, 20, or 21 days. The pharmaceutical composition may be administered every second day during a treatment window of about: 15, 16, 17, 18, 19, 20, or 21 days. The pharmaceutical composition may be administered every third day during a treatment window of about: 15, 16, 17, 18, 19, 20, or 21 days. The pharmaceutical composition may be administered on every 2^{nd}-3^{rd} day during a treatment window of about: 15, 16, 17, 18, 19, 20, or 21 days. The pharmaceutical composition may be administered every 4^{th} day during a treatment course of about: 15, 16, 17, 18, 19, 20, or 21 days; or every 5^{th} day during a treatment course of about: 15, 16, 17, 18, 19, 20, or 21 days. A person may readily appreciate the minimum numbers of days in a treatment window when the pharmaceutical composition may be administered every 6^{th}, 7^{th}, etc. day over a treatment window of a finite number of days as described herein.

A pharmaceutical composition may be administered daily for a longer duration than 14 days and may be administered at least about: 15, 16, 17, 18, 19, 20, or at least 21 days. The pharmaceutical composition may be administered daily on each of the about: 15, 16, 17, 18, 19, 20, or 21 days. The pharmaceutical composition may be administered every second day during a treatment window of about: 15, 16, 17, 18, 19, 20, or 21 days. The pharmaceutical composition may be administered every third day during a treatment window of about: 15, 16, 17, 18, 19, 20, or 21 days. The pharmaceutical composition may be administered on every 2^{nd}-3^{rd} day during a treatment window of about: 15, 16, 17, 18, 19, 20, or 21 days. The pharmaceutical composition may be administered every 4^{th} day during a treatment course of about: 15, 16, 17, 18, 19, 20, or 21 days; or every 5^{th} day during a treatment course of about: 15, 16, 17, 18, 19, 20, or 21 days. A person may readily appreciate the minimum numbers of days in a treatment window when the pharmaceutical composition is administered every 6^{th}, 7^{th}, etc. day over a treatment window of a finite number of days as described herein.

A pharmaceutical composition may be administered in a treatment course daily for a longer duration than about 14 days or about 21 days and may be administered in a treatment course of about one month, about two months, or about three months. The pharmaceutical composition may be administered daily on each of a one month, two month, or three month treatment course. The pharmaceutical composition may be administered every second day during a treatment course of about one month, about two months, or about three months. The pharmaceutical composition may be administered every third day during a treatment course of about one month, about two months, or about three months. The pharmaceutical composition may be administered on every 2^{nd}-3^{rd} day during a treatment course of about one month, about two months, or about three months. The pharmaceutical composition may be administered every 4^{th} day during a treatment course of about one month, about two months, or about three months; or every 5^{th} day during a treatment course of about one month, about two months, or about three months s. A person may readily appreciate the minimum numbers of days in a treatment course when the pharmaceutical composition is administered every 6^{th}, 7^{th}, etc. day over a treatment window of a finite number of days as described herein.

By way of non-limiting example, a longer treatment window with a decreased dose per day may be a treatment option for a subject. By way of example, the stage or severity of the cancer or tumor may indicate that a longer term course may provide clinical benefit. The pharmaceutical composition may be administered daily, or optionally, every other day (every 2^{nd} day) or every 3^{rd} day, or greater interval (i.e., every 4^{th} day, 5^{th} day, 6^{th} day) during a treatment course of about 1-2 weeks (e.g., about 5-14 days), about 1-3 weeks (e.g., about 5-21 days), about 1-4 weeks (e.g., about 5-28 days, about 5-36 days, or about 5-42 days, 7-14 days, 7-21 days, 7-28 days, 7-36 days, or 7-42 days; or 9-14 days, 9-21 days, 9-28 days, 9-36 days, or 9-42 days. The treatment course may be between about 1-3 months. The pharmaceutical composition may be administered daily for at least five days. The pharmaceutical composition may be administered daily for 5-14 days. The pharmaceutical composition may be administered for at least seven days, for example, for 7-14, 7-21, 7-28 days, 7-36 days, or 7-42 days. The pharmaceutical composition may be administered for at least nine days, for example, for 9-14 days, 9-21 days, 9-28 days, 9-36 days, or 9-42 days.

Even though as discussed herein and above, a treatment course comprising administering a pharmaceutical composition provides clinical benefit, a treatment course may be repeated with a time interval between each treatment course when the pharmaceutical composition may not be administered (i.e., non-treatment interval, off-drug treatment). A treatment cycle as described herein and in the art may comprise a treatment course followed by a non-treatment interval. A treatment cycle may be repeated as often as needed. For example, a treatment cycle may be repeated at least once, at least twice, at least three times, at least four times, at least five times, or more often as needed. A treatment cycle may be repeated once (i.e., administration of the pharmaceutical composition comprises 2 treatment cycles). The treatment cycle may be repeated twice or repeated 3 or more times. Accordingly, one, two, three, four, five, six, seven, eight, nine, ten, or more treatment cycles of treatment with a pharmaceutical composition may be performed. A treatment course or a treatment cycle may be repeated, such as when the cancer or tumor recurs, or when symptoms or sequelae of the disease or disorder that were significantly diminished by one treatment course as described above may have increased or may be detectable, or when the symptoms or sequelae of the disease or disorder may be exacerbated, a treatment course may be repeated. In other embodiments when the pharmaceutical composition is administered to a subject to prevent (i.e., reduce likelihood of occurrence or development) or to delay onset, progression, or severity of the cancer or tumor, a subject may receive the pharmaceutical composition over two or more treatment cycles. Accordingly, one cycle of treatment may be followed by a subsequent cycle of treatment. Each treatment course of a treatment cycle or each treatment course of two or more treatment cycles may be typically the same in duration and dosing of the pharmaceutical composition. In other embodiments, the duration and dosing of the pharmaceutical composition during each treatment course of a treatment cycle may be adjusted as determined by a person skilled in the medical art depending, for example, on the particular disease or disorder being treated, the pharmaceutical composition being administered, the health status of the subject and other relevant factors, which are discussed in greater detail herein. Accordingly, a treatment course of a second or any subsequent treatment cycle may be shortened or lengthened as deemed medically necessary or prudent. In other words, as may be appreciated by a person, each treatment course of two or more treatment cycles may be independent and the same or different; and each non-treatment interval of each treatment cycle may be independent and the same or different.

As described herein, each course of treatment in a treatment cycle may be separated by a time interval of days, weeks, or months without treatment with a pharmaceutical composition (i.e., non-treatment time interval or off-drug interval; called non-treatment interval herein). The non-treatment interval (such as days, weeks, months) between one treatment course and a subsequent treatment course may be typically greater than the longest time interval (i.e., number of days) between any two days of administration in the treatment course. By way of example, if a treatment course may be no longer than 14 days and the agent may be administered every other day during this treatment course, the non-treatment interval between two treatment courses may be greater than 2 days, such as 3, 4, 5, 6, 7, 8, 9, 10, 11, 12, 13, or 14 days or about 3 weeks, about 4 weeks, about 6 weeks, or about 2 months or longer as described herein. The non-treatment interval between two treatment courses may be about 5 days, about 1 week, about 2 weeks, about 3 weeks, about 1 month, about 6 weeks, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. The non-treatment interval may be about 2 years or about 3 years. The non-treatment time interval may be at least about 14 days, at least about 21 days, at least about 1 month, at least about 2 months, at least about 3 months, at least about 4 months, at least about 5 months, at least about 6 months, or at least about 1 year. A course of treatment (whether daily, every other day, every 3^{rd} day, or other interval between administrations within the treatment course as described above (e.g., 1-14 days, 2-14 days, 2-21 days, or 1-21 days)) may be administered about every 14 days (i.e., about every 2 weeks) (i.e., 14 days without pharmaceutical composition treatment), about every 21 days (i.e., about every 3 weeks), about every 28 days (i.e., about every 4 weeks), about every one month, about every 36 days, about every 42 days, about every 54 days, about every 60 days, or about every month (about every 30 days), about every two months (about every 60 days), about every quarter (about every 90 days), or about semi-annually (about every 180 days). A course of treatments (e.g., by way of non-limiting example, administration on at least one day or on at least two days during a course for about 2-21 days, about 2-14, days, about 5-14 days, about 7-14 days, about 9-14 days, about 5-21 days, about 7-21 days, about 9-21 days) may be administered every 28 days, every 36 days, every 42 days, every 54 days, every 60 days, or every month (about every 30 days), every two months (about every 60 days), every quarter (about every 90 days), or semi-annually (about every 180 days), or about every year (about 12 months). In other embodiments, a course of treatment (such as by way of non-limiting examples, e.g., for about 5-28 days, about 7-28 days, or about 9-28 days whether daily, every other day, every 3^{rd} day, or other interval between administrations within the treatment course) may be administered every 36 days, 42 days, 54 days, 60 days, or every month (about every 30 days), every two months (about every 60 days), every quarter (about every 90 days), or semi-annually (about every 180 days). A course of treatment (e.g., for about 5-36 days, 7-36 days, or 9-36 days whether daily, every other day, every 3^{rd} day, or other interval between administrations within the treatment course) may be administered every 42 days, 54 days, 60 days, or every month (about every 30 days), every two months (about every 60 days), every quarter (about every 90 days), or semi-annually (about every 180 days), or about every year (about 12 months).

The treatment course may be one day and the non-treatment interval may be at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. The treatment course may be at least two days or may be at least 3 days and no longer than 10 days, and the non-treatment interval may be at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. The treatment course may be at least three days and no longer than 10 days, no longer than 14 days, or no longer than 21 days, and the non-treatment interval may be at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. A treatment course (e.g., for about 5-42, 7-42, or 9-42 days whether daily, every other day, every 3^{rd} day, or other interval between administrations within the treatment course) may be administered every 42 days, 60 days, or every month (about every 30 days), every two months (about every 60 days), every quarter (about every 90 days), or semi-annually (about every 180 days), or about every year (about 12 months). The pharmaceutical composition may be administered daily for 5-14 days every 14 days (about every 2 weeks), or every 21-42 days. The pharmaceutical composition may be administered daily for 5-14 days quarterly. The pharmaceutical composition may be administered daily for 7-14 days every 21-42 days. The pharmaceutical composition may be administered daily for 7-14 days quarterly. The pharmaceutical composition may be administered daily for 9-14 days every 21-42 days or every 9-14 days quarterly. The non-treatment interval may vary between treatment courses. By way of non-limiting example, the non-treatment interval may be 14 days after the first course of treatment and may be 21 days or longer after the second, third, or fourth (or more) course of treatment. The pharmaceutical composition may be administered to the subject in need thereof once every 0.5-12 months. The pharmaceutical composition may be administered to the subject in need once every 4-12 months.

A pharmaceutical composition may be administered to a subject to reduce the likelihood or the risk that the subject may develop a cancer. The pharmaceutical composition may be administered for one or more days (e.g., any number of consecutives days between and including 2-3, -4, -5, -6, -7, -8, -9, -10, -11, -12, -13, -14, -15, -16, -17, -18, -19, -20, and 2-21 days) every 3, 4, 5, 6, 7, 8, 9, 10, 11, or 12 months. The pharmaceutical composition may be administered for one or more days (e.g., any number of consecutives days between and including 1-9 days) every 5 or 6 months.

The total daily dose of a pharmaceutical composition may be delivered as a single dose or as multiple doses on each day of administration. When multiple cycles of the pharmaceutical composition may be administered, the dose of a pharmaceutical composition administered on a single day may be less than the daily dose administered if only a single treatment course may be intended to be administered.

### Combination Therapies

A composition comprising a therapeutically effective amount of an anti-programmed cell death protein 1 agent and an immunotherapeutic agent, may also be used in combination with other therapeutic agents that may be selected for their therapeutic value for the condition to be treated. In general, the compositions described herein and, in embodiments where combinational therapy may be employed, other agents do not have to be administered in the same pharmaceutical composition, and may, because of different physical and chemical characteristics, have to be administered by different routes. The determination of the mode of administration and the advisability of administration, where possible, in the same pharmaceutical composition, may be within the knowledge of the clinician. The initial administration may be made according to established protocols recognized in the field, and then, based upon the observed effects, the dosage, modes of administration and times of administration may be modified by the clinician.

In some embodiments, it may be appropriate to administer at least one pharmaceutical composition described herein in combination with another therapeutic agent. Or, by way of example only, the therapeutic effectiveness of one of the pharmaceutical compositions described herein may be enhanced by administration of an adjuvant, i.e., by itself the adjuvant may have minimal therapeutic benefit, but in combination with another therapeutic agent, the overall therapeutic benefit to the subject may be enhanced. Or, by way of example only, the benefit experienced by a subject may be increased by administering one of the pharmaceutical compositions described herein with another therapeutic agent, which also includes a therapeutic regimen that also has therapeutic benefit. In any case, regardless of the disease, disorder or condition being treated, the overall benefit experienced by the subject may simply be additive of the two agents or the subject may experience a synergistic benefit.

The particular choice of pharmaceutical compositions used may depend upon the diagnosis of the attending physicians and their judgment of the condition of the subject and the appropriate treatment protocol. The pharmaceutical compositions may be administered concurrently (e.g., simultaneously, essentially simultaneously or within the same treatment protocol) or sequentially, depending upon the nature of the disease, disorder, or condition, the condition of the subject, and the actual choice of pharmaceutical compositions used. The determination of the order of administration, and the number of repetitions of administration of each therapeutic agent during a treatment protocol, may be well within the knowledge of the physician after evaluation of the disease being treated and the condition of the subject.

Therapeutically-effective dosages may vary when the drugs may be used in treatment combinations. Methods for experimentally determining therapeutically-effective dosages of drugs and other agents for use in combination treatment regimens may be described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the subject.

For combination therapies described herein, dosages of the co-administered pharmaceutical compositions may vary depending on the type of co-drug employed, on the specific drug employed, on the disease or condition being treated and so forth. In addition, when co-administered with one or more biologically active agents, the pharmaceutical composition provided herein may be administered either simultaneously with the pharmaceutical composition, or sequentially. If administered sequentially, the attending physician may decide on the appropriate sequence of administering the pharmaceutical composition in combination with a biologically active agent(s).

In one embodiment, the composition comprising a therapeutic therapeutically effective amount of an anti-programmed cell death protein 1 agent and an immunotherapeutic agent may be administered in any order or even simultaneously. In one embodiment, the composition comprising a therapeutic therapeutically effective amount of an anti-programmed cell death protein 1 agent and an immunotherapeutic agent may be administered in any order or even simultaneously, wherein the immunotherapeutic agent may be tabalumab (CAS number 1143503-67-7) or a fragment or a salt thereof. If simultaneously, the multiple therapeutic agents may be provided in a single, unified form, or in multiple forms, by way of example only, either as a single pill or as two separate pills. One of the therapeutic agents may be given in multiple doses, or both may be given as multiple doses. If not simultaneous, the timing between the multiple doses may vary from more than zero weeks to less than four weeks. In addition, the combination methods, compositions and formulations may be not to be limited to the use of only two agents; the use of multiple therapeutic combinations may be also envisioned.

It is understood that the dosage regimen to treat, prevent, or ameliorate the condition(s) for which relief is sought, may be modified in accordance with a variety of factors. These factors include the disorder or condition from which the subject suffers, as well as the age, weight, sex, diet, and medical condition of the subject. Thus, the dosage regimen actually employed may vary widely and therefore may deviate from the dosage regimens set forth herein.

The pharmaceutical compositions which make up the combination therapy disclosed herein may be a combined dosage form or in separate dosage forms intended for substantially simultaneous administration. The pharmaceutical compositions that make up the combination therapy may also be administered sequentially, with either pharmaceutical composition being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical composition, such as potency, solubility, bioavailability, plasma half-life and kinetic profile of the pharmaceutical composition. Circadian variation of the target molecule concentration may also determine the optimal dose interval.

In addition, the pharmaceutical compositions described herein also may be used in combination with procedures that may provide additional or synergistic benefit to the subject. By way of example only, subjects may be expected to find therapeutic and/or prophylactic benefit in the methods described herein, wherein pharmaceutical composition of a pharmaceutical composition disclosed herein and/or combinations with other therapeutics may be combined with genetic testing to determine whether that individual may be a carrier of a mutant gene that may be known to be correlated with a disease or condition.

The pharmaceutical compositions described herein and combination therapies may be administered before, during or after the occurrence of a disease or condition, and the timing of administering the composition containing a pharmaceutical composition may vary. Thus, for example, the pharmaceutical composition may be used as a prophylactic and may be administered continuously to subjects with a propensity to develop conditions or diseases in order to prevent the occurrence of the disease or condition. The pharmaceutical compositions and compositions may be administered to a subject during or as soon as possible after the onset of the symptoms. The administration of the pharmaceutical compositions may be initiated within the first 48 hours of the onset of the symptoms, such as within the first 48 hours of the onset of the symptoms, such as within the first 6 hours of the onset of the symptoms, such as within 3 hours of the onset of the symptoms. The initial administration may be via any route practical, such as, for example, an intravenous injection, a bolus injection, infusion over about 5 minutes to about 5 hours, a pill, a capsule, transdermal patch, buccal delivery, and the like, or combination thereof. A pharmaceutical composition may be administered as soon as may be practicable after the onset of a disease or condition may be detected or suspected, and for a length of time necessary for the treatment of the disease, such as, for example, from 1 day to about 3 months. The length of treatment may vary for each subject, and the length may be determined using the known criteria. For example, the pharmaceutical composition or a formulation containing the pharmaceutical composition may be administered for at least 2 weeks, such as about 1 month to about 5 years.

A pharmaceutical composition described herein may reduce the likelihood of a cancer in a subject in need thereof. The pharmaceutical composition described herein may be administered one or more days within a window of treatment. In some embodiments, the treatment window may be about: 1, 2, 3, 4, 5, 6, 7, 8, 9, 10, 11, 12, 13, 14, 15, 16, 17, 18, 19, 20, or 21 days. In some embodiments, a pharmaceutical composition described herein may be administered on two or more days within a treatment window of no longer than 7 days or 14 days; on 3 or more days within a treatment window of no longer than 7 days or 14 days; on 4 or more days within a treatment window of no longer than 7 days or 14 days; on 5 or more days within a treatment window of no longer than 7 days or 14 days; or on 6, 7, 8, 9, 10, 11, 12, 13, or 14 days within treatment window of no longer than 7 days or 14 days.

Chemotherapy and radiotherapy treatment regimens may comprise a finite number of cycles of on-drug therapy followed by off-drug therapy, or comprise a finite timeframe in which the chemotherapy or radiotherapy may be administered. The protocols may be determined by clinical trials, drug labels, and clinical staff in conjunction with the subject to be treated. The number of cycles of a chemotherapy or radiotherapy or the total length of time of a chemotherapy or radiotherapy regimen may vary depending on the subject's response to the cancer therapy. A pharmaceutical composition described herein may be administered after the treatment regimen of chemotherapy or radiotherapy has been completed.

### Indications

In some embodiments, a method of the current disclosure may be used to treat a cancer or a tumor. The cancer or tumor may be malignant. The cancer or tumor may be present in an organ. The cancer or tumor may be present in the head or neck region, the abdominal region, an upper limb, a lower limb, the skin, blood, the digestive tract, a germ cell, or the nervous system of a subject. The cancer or tumor may be present in at least one of: the blood, the lymph, or the cerebral spinal fluid.

In some embodiments, the tumor may be a solid tumor. The tumor may be a liquid tumor. Cancers that are liquid tumors may be those that occur, for example, in blood, bone marrow, and lymph nodes, and may include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Cancers that may be solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein may be metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

In some embodiments, a method of the current disclosure may be used to treat cancer, wherein the cancer may be prostate cancer, testicular cancer, breast cancer, brain cancer, pancreatic cancer, colon cancer, thyroid cancer, stomach cancer, lung cancer, melanoma, multiple myeloma, Hodgkin's lymphoma, or ovarian cancer.

The methods described herein may also be used for inhibiting progression of metastatic cancer tumors. Non-limiting examples of cancers include adrenocortical carcinoma, childhood adrenocortical carcinoma, aids-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac tumors, cervical cancer, childhood cervical cancer, childhood chordoma, chronic myeloproliferative disorders, colon cancer, colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (cml), hairy cell leukemia, lip cancer, liver cancer (primary), childhood liver cancer (primary), lobular carcinoma in situ (LCIS), lung cancer, non-small cell lung cancer, small cell lung cancer, lymphoma, aids-related lymphoma, burkitt lymphoma, cutaneous t-cell lymphoma, Hodgkin lymphoma, non-Hodgkin lymphoma, primary central nervous system lymphoma (CNS), melanoma, childhood melanoma, intraocular melanoma, Merkel cell carcinoma, malignant mesothelioma, childhood malignant mesothelioma, metastatic squamous neck cancer with occult primary, midline tract carcinoma involving NUT gene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleuropulmonary blastoma, prostate cancer, rectal cancer, renal pelvis transitional cell cancer, retinoblastoma, salivary gland cancer, childhood salivary gland cancer, Ewing sarcoma family of tumors, Kaposi Sarcoma, osteosarcoma, rhabdomyosarcoma, childhood rhabdomyosarcoma, soft tissue sarcoma, uterine sarcoma, Sezary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, and Waldenström macroglobulinemia.

In some embodiments, the cancer or tumor may be squamous non-small cell lung cancer, adeno non-small cell lung cancer, colorectal cancer, head and neck squamous cell carcinoma, breast cancer, or melanoma.

In some embodiments, the cancer or tumor contacts a blood vessel. In some embodiments, the cancer or the tumor may be in the interior of a blood vessel.

In some embodiments, at least one of the anti-programmed cell death protein 1 agent, the anti-programmed death ligand 1 agent, the anti-programmed death ligand 2 agent, or a salt of any one thereof, or any combination thereof; and the immunotherapeutic agent or salt thereof may be isolated and purified.

In some embodiments, the administration may be oral. In some embodiments, the administration may be topical, intravenous, intramuscular, or spinal. In some embodiments, the administration may be administered directly to the cancer or the tumor. In some embodiments, the administration may be an administration at a location different from the cancer or the tumor. In some embodiments, the subject has been previously diagnosed with the cancer or the tumor. In some embodiments, the cancer or the tumor may be a solid tumor or a solid cancer. In some embodiments, the cancer or the tumor may be a liquid cancer or a liquid tumor. In some embodiments, the cancer or the tumor may be malignant. In some embodiments, the cancer or the tumor may be present in an organ. In some embodiments, the tumor or the cancer may be present in at least one of: blood, lymph, cerebral spinal fluid. In some embodiments, the cancer or the tumor may be located in a head or neck region, abdominal region, a upper limb, a lower limb, skin, blood, digestive tract, germ cell, or nervous system.

### Cancer Interactomes

In some embodiments, a method described herein may be used to define a cancer interactome in a tumor microenvironment. A method may be used to correlate interaction partners of cells within a tumor.

To define a cancer interactome, a cell or a plurality of cells may be imaged. The cell or plurality of cells may be stained prior to imaging. An image may comprise at least about: 1, 2, 3, 4, 5, 6, 7, 8, 9, 10, 15, 20, 30, 40, 50, 60, 70, 80, 90, or 100 cells. In some embodiments, an image may comprise 1 to 10 cells, or 2-9 cells, or 3-8 cells. In some embodiments, an image may comprise 6 cells.

A cell image may comprise of a number of pixels. The number of pixels in a cell image may be at least about: 100, 1,000, 10,000, 100,000, 1,000,000, 1,500,000, or 2,000,000 pixels. The number of pixels in a cell image may be at least about 1,500,000, pixels.

A matrix may be generated based on the stained plurality of cells. The matrix may be used to define or represent the boundaries of the cells. In some embodiments, an area that may be a cell boundary may be defined using the number 1 in a matrix, and an area that may be not a cell boundary may be defined using the number 0. Thus, a matrix may represent cellular segmentation.

A matrix may be generated to represent an intensity value of cells in a tumor microenvironment. The matrix may be determined from a fluorescence readout. The matrix may be generated by a human or by a computer or by a software program. The intensity matrix may be predetermined, or may be obtained from a reference sample. Mixed or unmixed images may be used as inputs that may be processed and formatted into unmixed matrixes.

The intensity of each cell may be calculated for total readout intensity. A reference intensity may exist, wherein the reference intensity may be based on a prior sample that has been designated as a standard. The intensity of a cell may be compared to a reference intensity. After comparison of the intensity of a cell to a reference intensity, the cell may be labeled as having a positive intensity. After comparison of the intensity of a cell to a reference intensity, the cell may be labeled as having a negative intensity.

A pixel adjacency graph may be generated. Pixel adjacency may be defined as any two pixels that may be within a distance of square-root(2) pixels. Pixels that may be directly adjacent or diagonally adjacent may be considered as having adjacency.

A cell adjacency graph may be generated. Cellular adjacency may be defined as any two cells that may be within a distance of square-root(2) cells. Cells that may be directly adjacent or diagonally adjacent may be considered as having adjacency.

An adjacency heatmap may be generated from a cell adjacency graph. The cell adjacency heatmap generated from a tumor sample of a subject may then be compared to a reference heatmap.

A reference heatmap may be generated prior to diagnosing a subject. Reference heatmaps may be generated and used as references to predict subject treatment outcome. Reference heatmaps may be used as references to diagnose subjects in need of treatment.

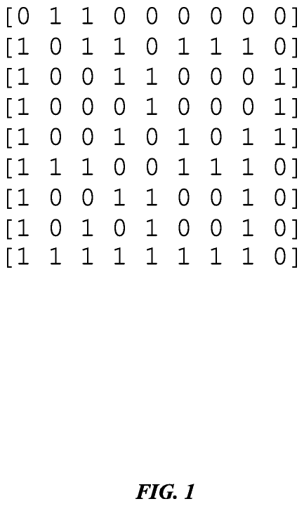

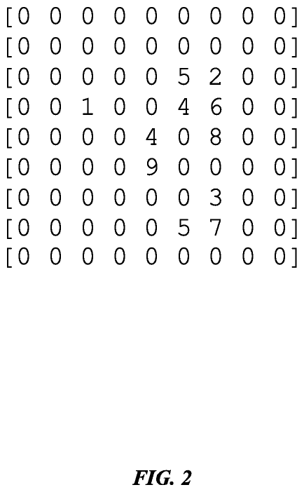

Two inputs may be used to define an interactome. The first is a matrix representing the cellular image where boundaries between cells are defined. FIG. 1 provides a representative example matrix that defines cellular boundaries. The matrix represents a 9×9 image which was determined to be comprised of six cells. FIG. 2 provides a 9×9 representative example which corresponds with FIG. 1 and is an example of an intensity matrix, wherein the relative intensity of each marker of interest per pixel is defined.

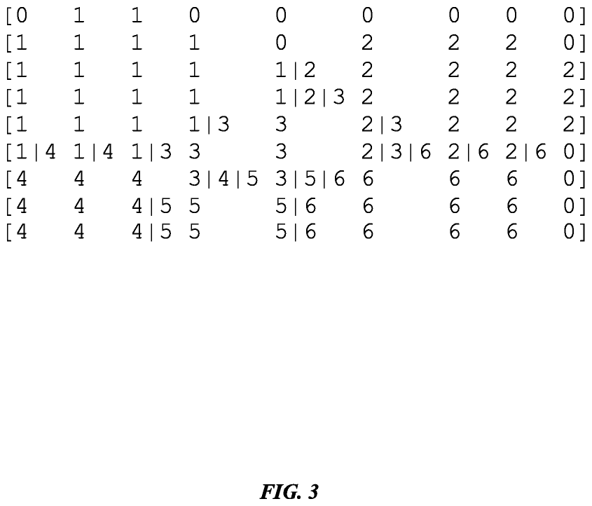

Based on the boundaries defined in the cellular boundary input, each pixel is labeled based on the cellular boundary with which it is contained. Boundary pixels may be labeled based on all cells sharing that boundary pixel. An example of pixel labeling is shown in FIG. 3. The matrix represents cell labeling from the boundary information from FIG. 1, wherein the symbol “I” indicates pixels that may be boundary pixels and labeled by the cells that may be part of that boundary.

### Computer Control Systems

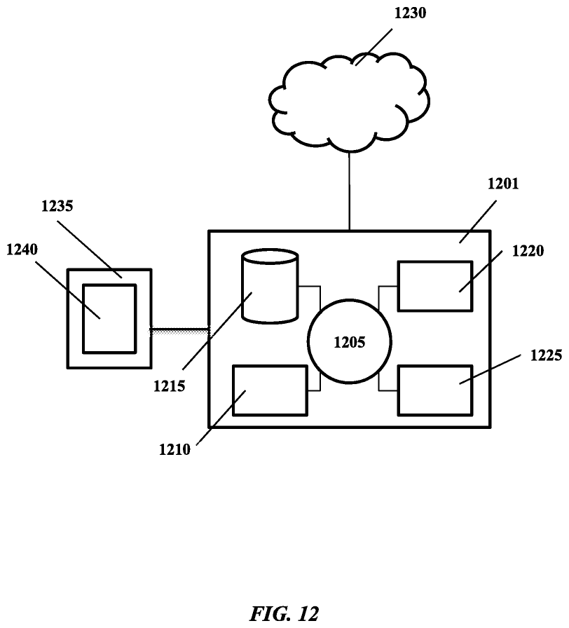

The present disclosure provides computer control systems that are programmed to implement methods of the disclosure. FIG. 12 shows a computer control system 1201. The computer control system 1201 may be implemented on an electronic device of a user or a computer system that is remotely located with respect to the electronic device. The electronic device may be a mobile electronic device.

The computer system 1201 includes a central processing unit (CPU, also “processor” and “computer processor” herein) 1205, which may be a single core or multi core processor, or a plurality of processors for parallel processing. The computer control system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 may be a data storage unit (or data repository) for storing data. The computer control system 1201 may be operatively coupled to a computer network (“network”) 1230 with the aid of the communication interface 1220. The network 1230 may be the Internet, an internet and/or extranet, or an intranet and/or extranet that is in communication with the Internet. The network 1230 in some cases is a telecommunication and/or data network. The network 1230 may include one or more computer servers, which may enable distributed computing, such as cloud computing. The network 1230, in some cases with the aid of the computer system 1201, may implement a peer-to-peer network, which may enable devices coupled to the computer system 1201 to behave as a client or a server.

The CPU 1205 may execute a sequence of machine-readable instructions, which may be embodied in a program or software. The instructions may be stored in a memory location, such as the memory 1210. The instructions may be directed to the CPU 1205, which may subsequently program or otherwise configure the CPU 1205 to implement methods of the present disclosure. Examples of operations performed by the CPU 1205 may include fetch, decode, execute, and writeback.

The CPU 1205 may be part of a circuit, such as an integrated circuit. One or more other components of the system 1201 may be included in the circuit. In some cases, the circuit is an application specific integrated circuit (ASIC).

The storage unit 1215 may store files, such as drivers, libraries and saved programs. The storage unit 1215 may store user data, e.g., user preferences and user programs. The computer system 1201 in some cases may include one or more additional data storage units that are external to the computer system 1201, such as located on a remote server that is in communication with the computer system 1201 through an intranet or the Internet.

The computer system 1201 may communicate with one or more remote computer systems through the network 1230. For instance, the computer system 1201 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 1201 via the network 1230.

Methods as described herein may be implemented by way of machine (e.g., computer processor) executable code stored on an electronic storage location of the computer system 1201, such as, for example, on the memory 1210 or electronic storage unit 1215. The machine executable or machine readable code may be provided in the form of software. During use, the code may be executed by the processor 1205. In some cases, the code may be retrieved from the storage unit 1215 and stored on the memory 1210 for ready access by the processor 1205. In some situations, the electronic storage unit 1215 may be precluded, and machine-executable instructions are stored on memory 1210.

The code may be pre-compiled and configured for use with a machine having a processor adapted to execute the code, or may be compiled during runtime. The code may be supplied in a programming language that may be selected to enable the code to execute in a pre-compiled or as-compiled fashion.

Aspects of the systems and methods provided herein, such as the computer system 1201, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 may include or be in communication with an electronic display 1235 that comprises a user interface (UI) 1240.

Methods and systems of the present disclosure may be implemented by way of one or more algorithms. An algorithm may be implemented by way of software upon execution by the central processing unit 1205.

### EXAMPLES

### Example 1: Staining a FFPE Tissue Section

A procedure is used to stain a FFPE tissue section mounted on a slide for multiple antibody/TSA-fluorophore conjugates. The immunohistochemistry staining is used with up to 6 different antibodies for multiple antigen detection on a tissue section on a single slide. This procedure allows quantitative analysis of antigens to identify biomarkers within a sample.

“IHC” refers to immunohistochemistry. “FFPE” refers to formalin fixed paraffin embedded. “TBS” refers to tris buffered saline. “TBS-T” refers to tris buffered saline with tween 20. “TSA” refers to tyramide signal amplification. “DAPI” refers to 4′, 6 diamidino-2-phenylindole, dihydrochloride.

A concentrated Hot Rinse at a ratio of 1:25 is formed (1 ml Hot Rinse to 24 ml of Deionized Water). A staining jar or metal slide canister is filled with 50-200 mL (enough volume to cover slides depending on size of staining jar) of 1× Hot Rinse. A second container is filled with 50-200 mL (enough volume to cover slides depending on size of staining jar) of 1× Borg Decloaker RTU. The decloaking Chamber is filled with 500 mL Deionized Water. The Borg Decloaker and Hot Rinse staining jars are placed into the Decloaking Chamber. The slides are placed into a slide rack, then the slide rack is placed into a staining jar containing Borg Decloaker. The decloaking chamber is heated at 110° C. for 15 minutes. The slide are transferred to the slide container with 1× Hot Rinse and dunked vigorously for a minimum of 20 times. A rinse is performed by gradually adding 500 mL of deionized water to the solution. One fourth of the hot rinse is poured out, and deionized water is added again. This is repeated until all of the deionized water is used.

The primary antibody is prepared prior to use for fluorescent IHC staining.

The slides are transferred to 1×TBS. 2.5 ml of 30% hydrogen peroxide in 247.5 ml of deionized water is added. The slides are transferred to 0.3% hydrogen peroxide and incubated for 10 minutes. A wash in 1×TBS is performed for 3 minutes.

Slides are transferred to 1×TBS-T, then laid flat into staining tray. 3-4 drops of antibody diluent in dispensed onto the slide to cover the tissue section completely, and incubated for at least 10 minutes. The primary antibody is prepared, incubated, and washed. The secondary antibody-RP is prepared, incubated, and washed.

### Example 2: Generation of a Spectral Library

A spectral library with the OPAL 7-color fIHC Kit (PerkinElmer, NEL797001kt) is generated to establish baseline fluorescent information for the performance of quantitative image analysis.

Slides on appropriate FFPE tissue are determined by study plan. The slides include: auto-fluorescent slide, DAPI only slide, Opal 520 only slide, Opal 540 only slide, Opal 570 only slide, Opal 620 only slide, Opal 650 only slide, and Opal 690 only slide.

A single antibody stain on FFPE tissue is performed. A heat induced stringent wash is performed.

One the slides have been mounted, a fluorescence image is captured and stored.

### Example 3: Image Acquisition

Images of fluorescent IHC stained slides are captured to visualize immune cells in situ in formalin-fixed paraffin-embedded (FFPE) tissue sections.

The image capture device is manually focused onto the tissue. The entire slide is scanned. The field resolution is set to 20×. An image is captured manually.

### Example 4: Quantitative Image Analysis at the Cell-Segmented Level

Images from stained slides of in vivo and tissue samples are generated and analyzed. Phenotypes are quantifies, discriminated, and analyzed with multiple biomarkers in situ in FFPE.

PerkinElmer Vextra 3 inForm software, PerkinElmer OPAL 7-color flHC kit, TSA reagent/antibodies, and FFPE control slides images are used.

FFPE control slides are as follows:

| | |
|---|---|
| Positive Control: | Non-tumor antibodies |
| Normal human tissue | |
| Positive Control: Tumor human | Tumor-specific antibodies |
| Negative Control (non-specific): | Host-specific isotype Antibody |
| Normal human tissue | in place of 1° Antibody, with 2° |
| | Antibody and TSA |
| Auto Fluorescent Control: | No 1° Antibodies, with all 2° |
| Sample Specific | Antibody and TSA non-tumor |
| | antibodies |

The fluorophores used in the study are selected. Cell segmentation settings are set. If the system erroneously counted cells, the parameters are adjusted. The image is phenotyped, with the filters adjusted accordingly to the phenotype and stain used. The cells that express the phenotype of interest are selected.

The nature of the native cancer interactome in a tumor microenvironment of tissues may be defined and quantified. For example, tissues of humans, murines, canines, bovines, filines, and equines may be defined and quantified. A method of multiplexed staining, followed by imaging analysis may be used to define and quantify the cancer interactome.

A number of benefits may be provided by quantifying the cancer interactome, such as: defining the relationship between distinct cell-types independently of cellular density, defining interaction partners and measuring changes in interaction partners based on clinical endpoints, defining cell-subpopulations that exist in their native context but remain unknown due to tumor analysis methods based on bulk-derived measurements or single cell assays, studying and determining relationships between phenotypically defined and phenotypically undetermined cell-types that are associated with a specific clinical endpoint (e.g., treatment outcome with anti-programmed cell death protein 1), and determining new insights based on cellular interactions that are not captured using other tumor analysis methods.

The following method may be used to define an interactome:

(1): Define Cell Boundaries and Label Matrix Vertices by Cell

Two inputs may be used to define an interactome. The first is a matrix representing the cellular image where boundaries between cells are defined. FIG. 1 provides a representative example matrix that defines cellular boundaries. The matrix represents a 9×9 image which was determined to be comprised of six cells. FIG. 2 provides a 9×9 representative example which corresponds with FIG. 1 and is an example of an intensity matrix, wherein the relative intensity of each marker of interest per pixel is defined.

Based on the boundaries defined in the cellular boundary input, each pixel is labeled based on the cellular boundary with which it is contained. Boundary pixels are labeled based on all cells sharing that boundary pixel. An example of pixel labeling is shown in FIG. 3. The matrix represents cell labeling from the boundary information from FIG. 1, wherein the symbol “I” indicates pixels that are boundary pixels and labeled by the cells that are part of that boundary.

(2): Measuring Concordance Between Single-Plex and Multiplex IHC Staining and Imaging

A key challenge in multiplexed IHC staining is determining the impact of multiplexing on the performance of any single antibody during testing. Single-plex and multiplex pixel intensity distributions are overlaid to confirm correspondence of staining intensity scales.

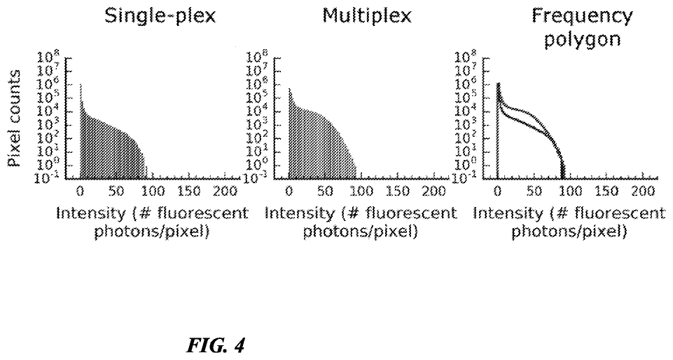

The following method is used to quantify the impact on intensity scales by comparing single-plex and multiplex staining thru frequency polygons. Frequency polygons of pixel staining intensities for example single-plex and multiplex images for a single channel are overlaid and visual inspection by study leadership confirms correspondence. An example is shown in FIG. 4. The distributions of positive pixels determined from (2) for a single channel for both single-plex and multiplex are compared using the following method. Median and Median Absolute Deviation (MAD) are calculated for both distributions. The distance between medians is divided by the MAD of both distributions and the larger of the two resulting normalized distances is reported. A maximum normalized distance of less than 2.0 is labeled acceptable, between 2.0 and 4.0 is labeled marginal and above 4.0 is labeled not acceptable.

(3): Defining Positive and Negative Intensity Thresholds for Each Fluorophore

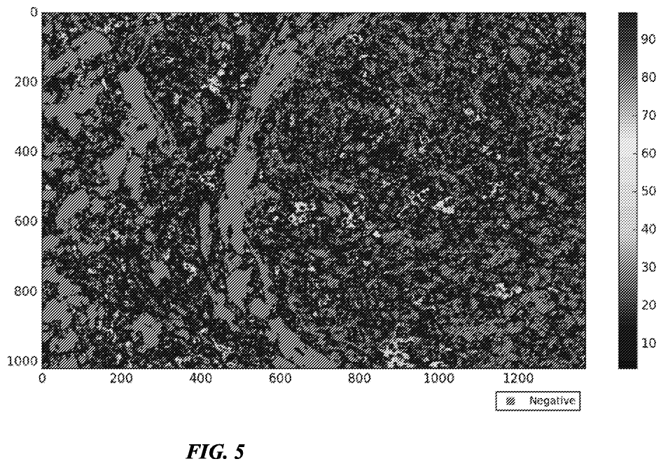

Raw data from fluorescence channels is provided using heatmap visualizations thresholded across the lower range of intensities. An appropriate threshold for positive staining is based on the marker of interest in a given study. FIG. 5 shows an example of positive intensity thresholding with heatmap visualization. A heatmap shows potential positive pixels for an image as intensities colored blue to red. Negative staining is shown in grey. Note that blue ranging 10-20 may also be defined as negative depending on positive and negative controls and the fluorophore's range of dynamic signal.

(4): Calculate Total Readout Intensity for Each Cell and Each Readout

Intensities of pixels labeled as positive using the thresholds from (2) within any cellular boundary (including boundary pixels) are summed over the cell area to provide the total marker intensity within the cell for each cell. This values are normalized for the total area of the cell.

(5): Label Cells as Positive or Negative for Each Readout

To accurately perform phenotyping, each the definition of positive/negative for a given marker is calibrated based on a reasonable sample of study data input from experimentalists. A set of calibration images is provided in which positive and negative may be performed manually or thru machine based learning. Phenotyping is calibrated by choosing an intensity distribution percentage cutoff where the distribution is calculated using the normalized intensity from (4) for all cells with any positive intensity in each image. The algorithm is cross-validated within the calibration set and a minimum of 90% accuracy is required to use the calibration.

(7): Determine Adjacent Pixel Adjacency Graph

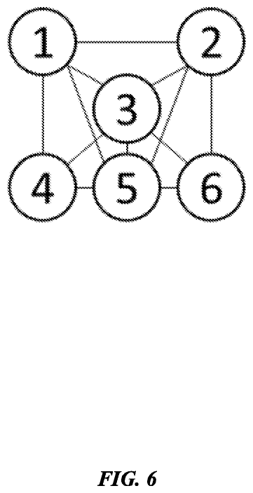

Image data (pixels and cells) may be converted into a mathematical graph that represents spatial adjacency relationships between the objects. Pixel adjacency may be defined as any two pixels that are within a distance of square-root(2) pixels, that is, direct adjacency or diagonal adjacency of any two pixels. A mathematical directed graph is constructed using this data where each node (vertex) represents a single pixel. Edges are added by iterating over all nodes and adding a single edge for each adjacent neighbor where the edge source is the node of interest and the edge target. In the case of pixels, the adjacency graph does not include pixels which are not positive for any marker of interest. An adjacency graph is shown in FIG. 6.

(8): Determine Cell Adjacency Graph

Cellular adjacency is defined as two cells where any two pixels with either cell (including boundary pixels) are within a distance of square-root(2) pixels. That is, direct adjacency or diagonal adjacency of any two pixels within the two cells is counted as cellular adjacency. An adjacency graph is created in a similar fashion to pixels where cells counted as adjacent are linked in a directed graph. Unlike the pixel data, cells which are not positive for any marker are included as “Other” cells.

(9): Calculate Fraction of Adjacency Between Pixels

Pixels are grouped according to intensity value positivity, where each group is a positive/negative set of one or more study readouts. Pixels may be part of more than one group. Pixel adjacency graphs are pooled across a given image set defined by the study objectives. For a given pair of pixel groups, the fraction of adjacency between a source group and target group is defined as (number edges with source as node in source group and target as node in target group)/(total number of edges with source as node in source group). Different image cohorts may contain different numbers of images and different numbers of pixels, but because fraction of adjacency is a normalized quantity, it is comparable regardless of these differences.

Fraction of adjacencies may be displayed when calculated all-by-all on a set of phenotype groups as heatmap. In these heatmaps, the source phenotype is presented on the rows and the target phenotype is presented on the columns. Because each row shares a source phenotype, all cells have within a row have an equal denominator. Each cell in the heatmap is calculated independently from each other cell.

(10): Calculate Fraction of Adjacency Between Cell-Types

Cells are grouped according to phenotypes of interest, where each phenotype is a positive/negative set of one or more study readouts. The adjacency heatmap is calculated using the same method as pixels.

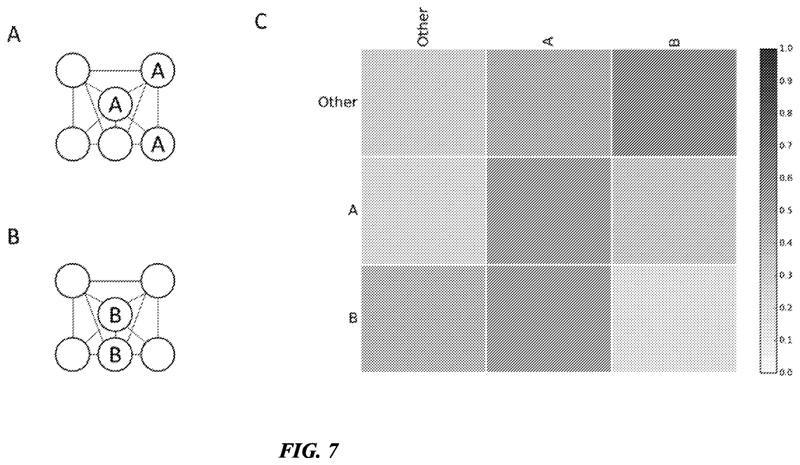

An example calculation and resulting heatmap are presented in FIG. 7 A-C. FIG. A gives an example cell adjacency network from FIG. 4 with cells phenotyped for readout “A”. FIG. B gives an example cell adjacency network from FIG. 4 with cells phenotyped with readout “B”.

FIG. C provides a calculated cell adjacency heatmap. It shows closer adjacency from A->A, B->A and from Other->B. Fractions of adjacency in a row may not add to 1 because of cells that are positive for multiple readouts.

(11): Calculate Differences in the Fraction of Adjacency Between Sample Groups.

Reference heat maps may be generated based on study objectives. In this instance, a reference heat map for non-responders and a reference heat map for responders may be generated.

If multiple image sets have been defined based on study objectives, fraction of adjacency differences may be calculated for each heatmap cell between two heatmaps of identical markers (either pixels or cells). The difference between fractions of adjacency for each source/target pair between a study reference image set and comparison set may be also reported as a heatmap. P-values for the differences may be calculated using Fisher's exact test by constructing a contingency table of the edge counts consisting of the two image sets.

FIG. 8 shows an adjacency heatmap of a subject that did not respond to treatment, or a non-responder subject. Fraction of adjacency heatmap were calculated on images taken from the invasive margin of subjects who were non-responsive to treatment. “Other” in this heatmap are cells which are not determined positive for any study readout. The heatmap shows high proximity between specific phenotypes such as CD20/CD20, CD19/CD20, and CD19/CD19.

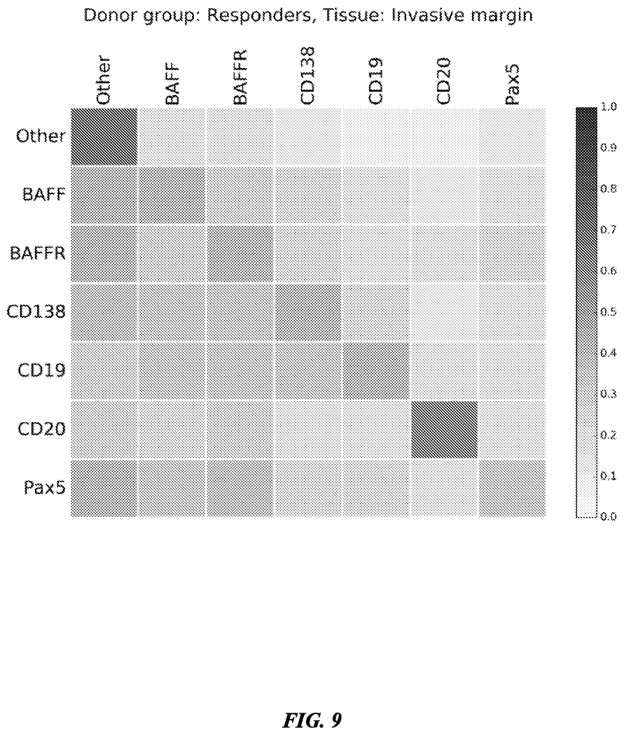

FIG. 9 shows a fraction of adjacency heatmap that was calculated on images taken from the invasive margin of subjects who were responsive to treatment. “Other” in this heatmap are cells which are not determined positive for any study readout.

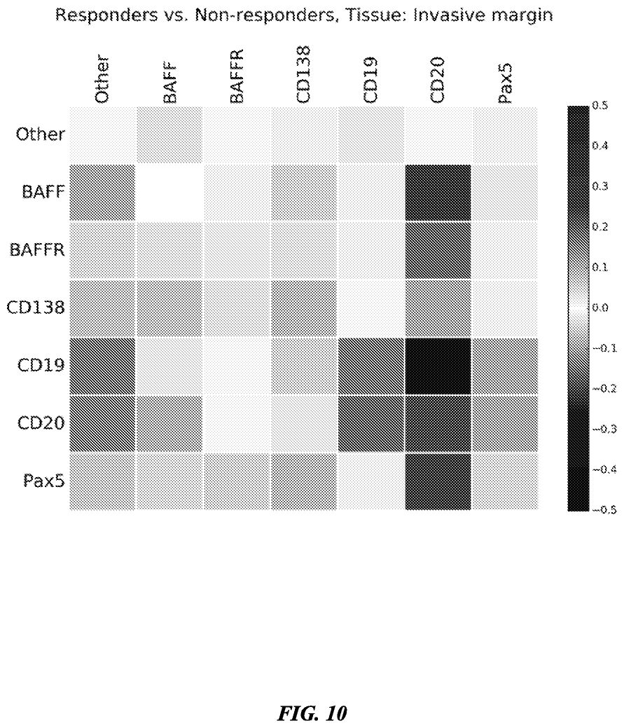

FIG. 10 shows an adjacency heatmap of the difference between the heatmap of a responder minus the heatmap of a non-responder. That is, the heatmap of FIG. 8 is subtracted from the heatmap of FIG. 9.

“Other” in this heatmap are cells which are not determined positive for any study readout. This heatmap shows lower proximity between CD20 and study phenotypes in responders as compared to non-responders and higher proximity between other cells and study phenotypes.

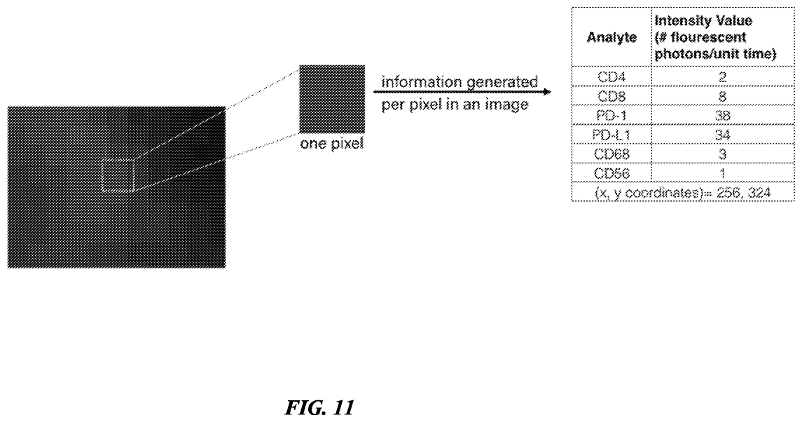

FIG. 11 illustrates the use of pixel-based analytics for diagnostic and therapeutic development. Each image of cells is comprised of approximately 1.5 million pixels where each pixel is defined in space according to x, y coordinate values and measures the relative intensity (e.g., relative expression level) of each analyte. FIG. 11 is a subsection of an image that was acquired on a tissue section tested for CD4, CD8, PD-1, PD-L1, CD68 and CD56 expression.

### Example 5: Antitumor Cell Efficacy of Compositions of the Disclosure

Cytotoxic activity of the compositions against human tumor cells are measured using an in vitro cell culture viability assay. Human A375 melanoma cells are incubated with 10 different composition concentrations between 10 nM and 100 μM for 72 hours and stained with a resazurin-based cell viability reagent to quantify viable cells. Living cells may reduce the non-fluorescent resazurin to a highly fluorescent resorufin, which may be quantified with a spectrophotometric plate reader. The fluorescence signal is normalized between the signals of untreated viable cells and the background signal of dead cells. Then, the half-maximum concentration for the inhibition of resazurin staining is calculated.

### Example 6: Cell Proliferation Assay

The ability of a composition of the present disclosure to inhibit the growth of cells, such as human leukemia, VCaP, LNCaP, 22RV1, DUi45, LNCaP-AR, MV4; 11, KOPN-8, ML-2, MOLM-13, bone marrow cells (BMCs), MLL-AF9, MLL-ENL, E2A-HLF, HL-60 and NB4 cells, is tested using a cell viability assay, such as the Promega CellTiter-Glo® Luminescent Cell Viability Assay (Promega Technical Bulletin, 2015, “CellTiter-Glo® Luminescent Cell Viability Assay”: 1-15, herein incorporated by reference in its entirety). Cells are plated at relevant concentrations, for example about 1×10⁵-2×10⁵ cells per well in a 96-well plate. A composition of the present disclosure is added at a concentration up to about 2 μM with eight, 2-fold serial dilutions for each composition. Cells are incubated at 37° C. for a period of time, for example, 72 hours, then cells in the control wells are counted. Media is changed to restore viable cell numbers to the original concentration, and compositions are re-supplied. Proliferation is measured about 72 hours later using Promega CellTiter-Glo® reagents, as per kit instructions.

### Example 7: Efficacy Study in Mouse Xenograft Tumor Model

Immunodeficient mice, such as 8-10 week-old female nude (nu/nu) mice, are used for in vivo efficacy studies in accordance with the guidelines approved by IACUC. Leukemia cells, such as human MV4-11 leukemia cells available from ATCC, are implanted subcutaneously via needle into female nude mice (5×10⁶ cells/mouse). When the tumor reaches a size of approximately 150 to 250 mm³ in mice, the tumor-bearing mice are randomly assigned to a vehicle control or composition treatment group (8 animals per group). Animals are treated with a composition of the present disclosure by oral gavage or intraperitoneal injection in an appropriate amount and frequency as may be determined by the skilled artisan without undue experimentation. Subcutaneous tumor volume in nude mice and mice body weight are measured twice weekly. Tumor volumes are calculated by measuring two perpendicular diameters with calipers (V=(length×width²)/2). Percentage tumor growth inhibition (% TGI=1−[change of tumor volume in treatment group/change of tumor volume in control group]*100) is used to evaluate anti-tumor efficacy. Statistical significance is evaluated using a one-tailed, two sample t test. P<0.05 is considered statistically significant.

### Example 8: Synergistic Efficacy Study in Mouse Xenograft Tumor Model

Immunodeficient mice, such as 8-10 week-old female nude (nu/nu) mice, are used for in vivo efficacy studies in accordance with the guidelines approved by IACUC. Leukemia cells, such as human MV4-11 leukemia cells available from ATCC, are implanted subcutaneously via needle into female nude mice (5×10⁶ cells/mouse). When the tumor reaches a size of approximately 150 to 250 mm³ in mice, the tumor-bearing mice are randomly assigned to one or four groups: a vehicle control, an anti-programmed cell death protein 1 monotherapy treatment group, a tabalumab treatment group, or a combination anti-programmed cell death protein 1/tabalumab treatment group (8 animals per group). All animals in the treatment groups are treated by oral gavage or intraperitoneal injection in an appropriate amount and frequency as may be determined by the skilled artisan without undue experimentation. Subcutaneous tumor volume in nude mice and mice body weight are measured twice weekly. Tumor volumes are calculated by measuring two perpendicular diameters with calipers (V=(length×width²)/2). Percentage tumor growth inhibition (% TGI=1−[change of tumor volume in treatment group/change of tumor volume in control group]*100) is used to evaluate anti-tumor efficacy. Statistical significance is evaluated using a one-tailed, two sample t test. P<0.05 is considered statistically significant. The change of tumor volume in the combination treatment group is larger than in the anti-programmed cell death protein 1 monotherapy treatment group, the tabalumab treatment group, and larger than the addition of the change of both groups separately.

### Example 9: Efficacy Study in Non-Small Cell Lung Cancer Xenograft Model

Immunodeficient mice, such as 4-6 week-old male CB17 severe combined immunodeficiency (SCID) mice, are used for in vivo efficacy studies in accordance with the guidelines approved by IACUC. Parental non-small cell lung cancer cells, such as VCaP or LNCaP-AR cells, are implanted subcutaneously into male CB.17.SCID mice (3-4×10⁶ cells in 50% Matrigel). When the tumor reaches a palpable size of approximately 80 mm³, the tumor-bearing mice are randomly assigned to a vehicle control or composition treatment group (6 or more animals per group). Animals are treated with a composition of the present disclosure by intraperitoneal injection in an appropriate amount and frequency as may be determined by the skilled artisan without undue experimentation. In one example, mice are treated with 40 mg/kg of a composition of the present disclosure daily by i.p. injection for two weeks, then 5 days per week thereafter. Subcutaneous tumor volume and mice body weight are measured twice weekly. Tumor volumes are calculated by measuring two perpendicular diameters with calipers (V=(length×width²)/2).

### Example 10: Efficacy Study in Castration-Resistant Non-Small Cell Lung Cancer Tumor Xenograft Model (VCaP)

Immunodeficient mice, such as 4-6 week-old male CB17 severe combined immunodeficiency (SCID) mice, are used for in vivo efficacy studies in accordance with the guidelines approved by IACUC. Parental non-small cell lung cancer cells, such as VCaP cells, are implanted subcutaneously into male CB.17.SCID mice (3-4×10⁶ cells in 50% Matrigel). When the tumor reaches a size of approximately 200-300 mm³, the tumor-bearing mice are physically castrated and tumors observed for regression and regrowth to approximately 150 mm³. The tumor-bearing mice are randomly assigned to a vehicle control or composition treatment group (6 or more animals per group). Animals are treated with a composition of the present disclosure by intraperitoneal injection in an appropriate amount and frequency as may be determined by the skilled artisan without undue experimentation. In one example, mice are treated with 40 mg/kg of a composition of the present disclosure daily by i.p. injection. Subcutaneous tumor volume and mice body weight are measured twice weekly. Tumor volumes are calculated by measuring two perpendicular diameters with calipers (V=(length×width²)/2).

### Example 11: Efficacy Study in Castration-Resistant Non-Small Cell Lung Cancer Xenograft Model (LNCaP-AR)

Immunodeficient mice, such as 4-6 week-old male CB17 severe combined immunodeficiency (SCID) mice, are used for in vivo efficacy studies in accordance with the guidelines approved by IACUC. CB.17.SCID mice are surgically castrated and allowed to recover for 2-3 weeks before implanting parental non-small cell lung cancer cells, such as LNCaP-AR cells, subcutaneously into (3-4×10⁶ cells in 50% Matrigel). When the tumor reaches a size of approximately 80-100 mm³, the tumor-bearing mice are randomly assigned to a vehicle control or composition treatment group (6 or more animals per group). Animals are treated with a composition of the present disclosure by intraperitoneal injection in an appropriate amount and frequency as may be determined by the skilled artisan without undue experimentation. In one example, mice are treated with 60 mg/kg of a composition of the present disclosure daily by i.p. injection for 27 days. Subcutaneous tumor volume and mice body weight are measured twice weekly. Tumor volumes are calculated by measuring two perpendicular diameters with calipers (V=(length×width²)/2).

### Example 12: Conservation of Type II Signature

Tumor samples were obtained from subjects with squamous non-small cell lung cancer, adeno non-small cell lung cancer, colorectal cancer, head and neck squamous cell carcinoma, breast cancer, or melanoma.

Tumor samples were stained and imaged, and determined to possess a Type II Resistant Signature.

### Example 13: Type II Signature

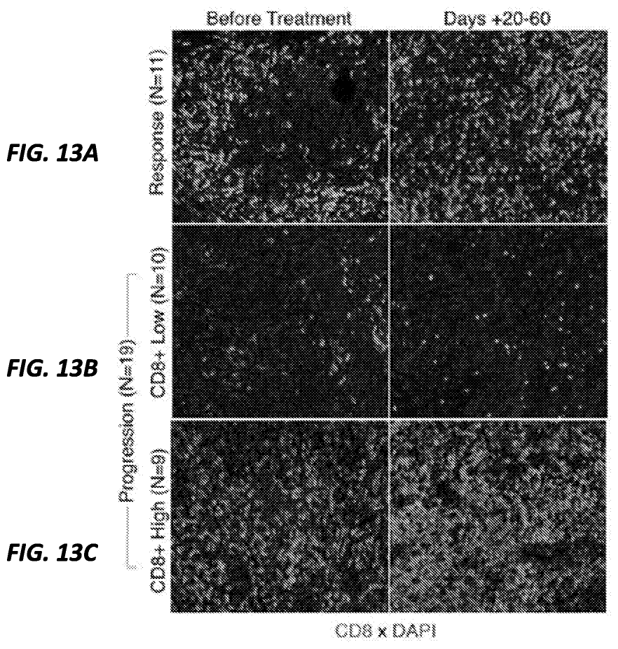

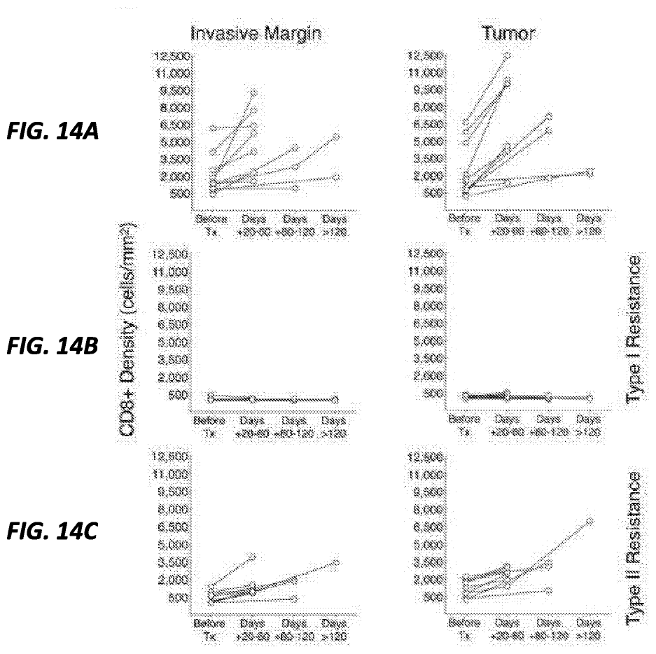

FIGS. 13A, 13B, and 13C, along with FIGS. 14A, 14B, and 14C, illustrate three types of tumors before and after treatment. FIG. 13A illustrates a traditionally “hot” tumor, with a high level of CD8 cells. This “hot” tumor responds to single agent treatment. Before treatment, the “hot” tumor is expected to respond to treatment. As illustrated in FIG. 14A, the amount of CD8 density increase during treatment at both the invasive margin and within the tumor itself.

FIG. 13B illustrates a traditionally “cold” tumor, one that is not expected to, and does not, respond to single agent treatment. The CD8 density at both the invasive margin and within the tumor (FIG. 14B) stays consistent and does not increase.

FIG. 13C illustrates a tumor that may be traditionally labeled as a “hot” tumor because of the high density of CD8 cells, and may be expected to respond to single agent therapy. However, this tumor does not respond to single agent therapy during treatment. The level of CD8 density does increase, as illustrated in FIG. 14C, and mimics a responder (FIG. 14A) pattern. Thus, FIG. 13C and FIG. 14C illustrate a Type II Signature or Type II Resistant tumor.

### Example 14: Proliferation of Type II Resistant Cells

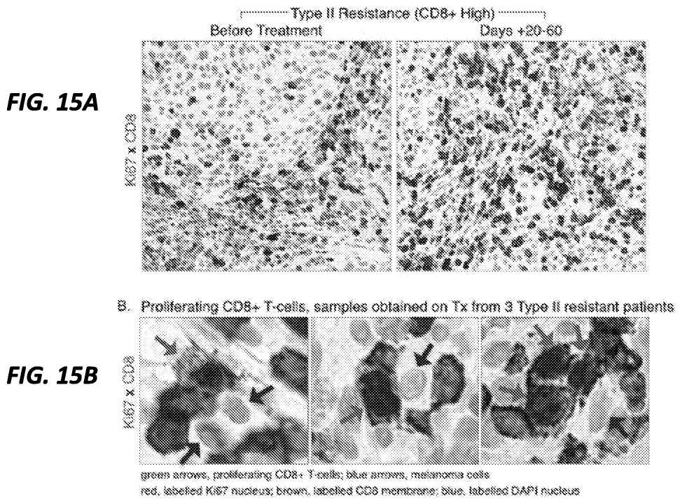

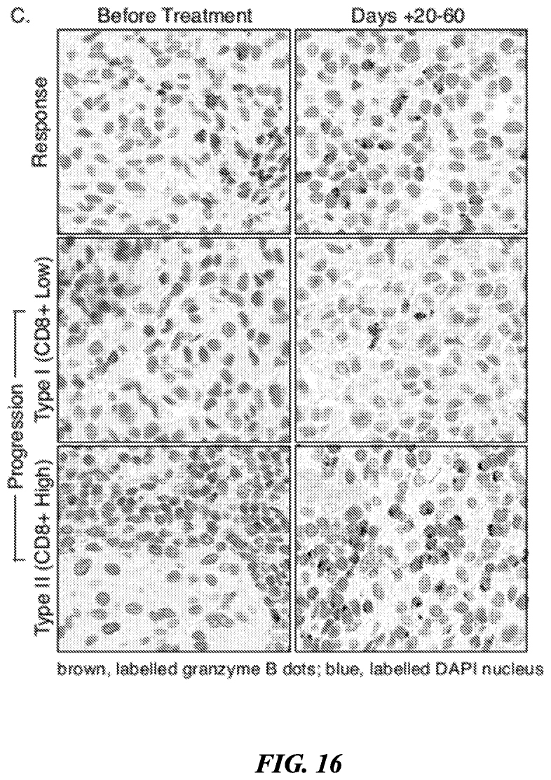

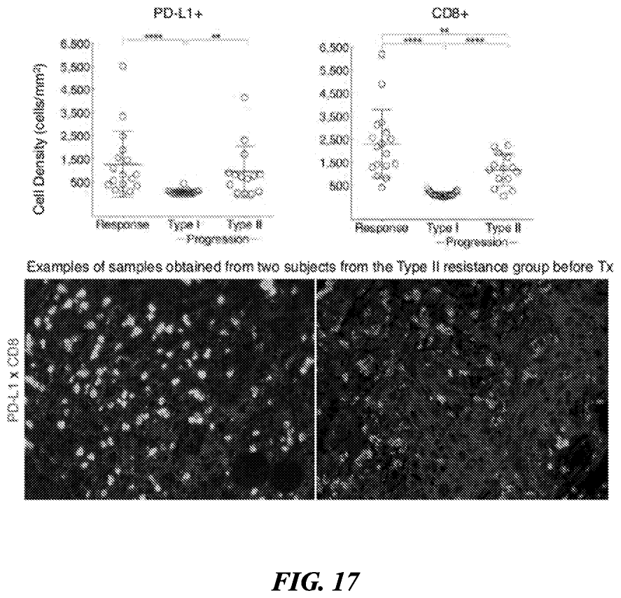

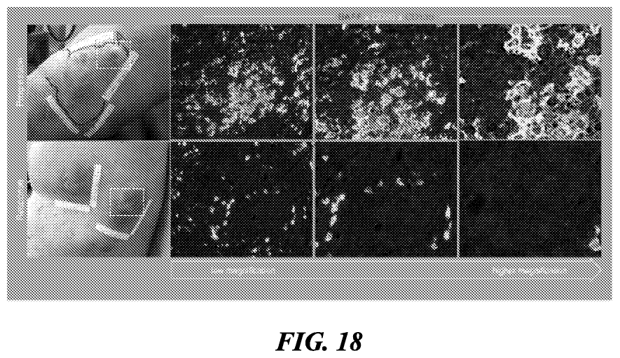

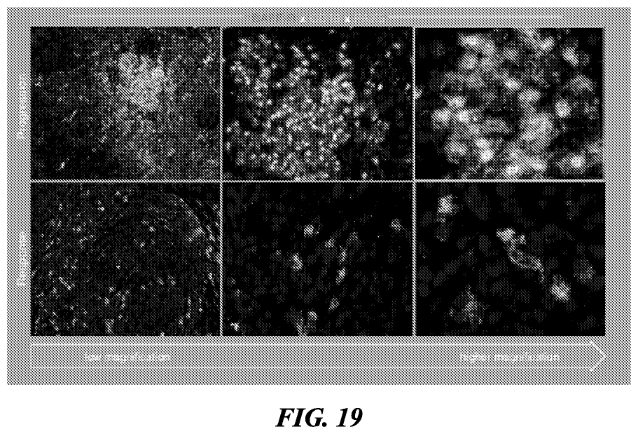

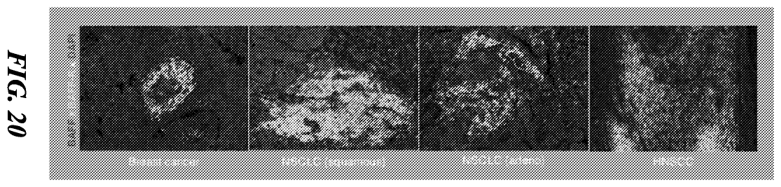

FIG. 15A and FIG. 15B illustrate Type II resistant tumors and the evidence of cell proliferation in these tumors before and during anti-PD1 treatment. FIG. 15B provides evidence for proliferating CD8 T-cells with samples obtained during treatment from three Type II resistant subjects. The T-cells are proliferating as well as turned on by secreting granzyme B.

### Example 15: Dewaxing and Antigen Retrieval

Dilute concentrated Hot Rinse at a ratio of 1:25. Fill staining jar (100 mL) or metal slide canister with (250 mL) of 1× Hot Rinse. Fill a second container with 100-250 mL of 1× Borg Decloaker RTU. Fill Decloaking Chamber with 500 mL Deionized Water. Heat to 110° C. for 15 minutes. When the solution reaches 80-125° C., the solution turns a faint purple color and indicates that the high temperature solution is at correct pH. A yellow or red solution indicates an incorrect pH. Transfer slides to the slide container with 1× Hot Rinse and dunk vigorously for a minimum of 20 times. Gently rinse by gradually adding Deionized Water to the solution. Use a total of 500 mL of Deionized Water in this step Pour off ¼ of Hot Rinse out of staining jar. Top off with Deionized Water, dunk vigorously for a minimum of 20 times. Repeat until all of the of Deionized Water is used. Hold slides in fresh Deionized Water until next step. Turn off decloaking chamber.

### Example 16: Endogenous Peroxidase Blocking

Prepare 250 ml of 0.3% Hydrogen Peroxide in a staining jar. Add 2.5 ml 30% Hydrogen Peroxide in 247.5 ml of Deionized Water. Transfer slides to 0.3% Hydrogen Peroxide, and dunk a minimum of 20 times. Incubate for 10 minutes+/−1 minute. Transfer slides to 1× TBS. Dunk vigorously for a minimum of 20 times to wash. Hold slides in 1×TBS until Fluorescent IHC Staining. Slides may be stored overnight at 2-8° C. in a staining jar containing fresh 1×TBS.

### Example 17: Antibody Incubation

Place slides into 1×TBS-T, and agitate at least 5 dips prior to staining. Incubate for at least 30 minutes no more than 1 hour. Remove excess Antibody by aspiration. Wash with fresh 1×TBS-T, and dunk vigorously. Allow slides to equilibrate for at least 3 min. Dunk again. Transfer to a new TBS-T wash, and repeat the dunk-equilibrate-dunk for a total of 4 washes. Hold slides in last wash until Secondary Antibody HRP Incubation.

### Example 18: Secondary Antibody-HRP Incubation

Apply 200 μL of Opal Polymer HRP Ms+Rb (Perkin Elmer). Incubate for at least 10 minutes and no more than 12 minutes. Remove excess Secondary Antibody by aspiration. Wash slides with 1×TBS-T prior to placing into slide holder, or place slides into slide holder and dunk at least 5 dips to pre-wash with 1×TBS-T. To wash, transfer slides to fresh 1×TBS-T, and dunk vigorously for a minimum of 20 times. Allow slides to equilibrate for at least 3 min. Dunk again for a minimum of 20 dips. Transfer to a new TBS-T wash, and repeat the dunk-equilibrate-dunk for a total of 3 washes. Hold slides in last wash until TSA incubation.

### Example 19: TSA Incubation

Apply 150 μL/slide of TSA Reagent. Incubate for at least 10 minutes and no more than 11 minutes. Remove excess TSA Reagent by aspiration. Irrigate slides with 1×TBS-T prior to placing into slide holder, or place slides into slide holder and dunk at least 5 dips to pre-wash with 1×TBS-T. To wash, transfer slides to fresh 1×TBS-T, and dunk vigorously for a minimum of 20 times. Allow slides to equilibrate for at least 3 min. Dunk again for a minimum of 20 dips. Transfer to a new TBS-T wash, and repeat the dunk-equilibrate-dunk for a total of 3 washes. Hold slides in last wash until Heat Induced Stringent Wash.

### Example 20: Heat Induced Stringent Wash

Prepare 250 mL of 1× AR6 Buffer from the 7-color Fluorescent IHC Kit (Perkin Elmer, NEL797001) using Deionized Water. Place the covered staining jar with slides into a 1000-Watt microwave oven, and place a second jar containing 250 mL Deionized Water to balance the heat distribution during the cycle. Heat in microwave. Gently cool by gradually adding 1×TBS to the jar. Pour off ¼ of Citrate buffer out of staining jar. Top off with 1×TBS and agitate a minimum of 10 dips. Repeat until 500 mL of TBS is used. Transfer slides into fresh 1×TBS wash, dip the slide rack up and down at least 5 times. Hold slides in 1×TBS before proceeding with next step. If multiplexing more antibodies proceed to section 15, Sequential IHC Staining for Multiplex.

### Example 21: Nuclear Counterstain

Prepare DAPI solution from the TSA kit at concentration of 2 drops/1 mL TBS-T. DAPI Counterstain. Transfer slides to staining jar filled with 1×TBS-T, and dunk at least 5 times. Apply 200 μL DAPI solution, and incubate in the staining tray for 5 minutes+/−1 minute. Transfer to a new wash of 1×TBS-T. Dunk a minimum of 20 times, and allow slides to equilibrate for 5 min. Dunk slides a minimum of 20 times. Transfer to a new wash of 1×TBS. Dunk a minimum of 20 times, and allow slides to equilibrate for 5 min. Dunk slides a minimum of 20 times. Transfer to a new wash of deionized water, and dunk a minimum of 20 times.

### Example 22: Correlation Between BAFF Expression and T-Cell Inflamed Signature

This aim of this study was to examine the relationship between BAFF expression and a T-cell inflamed signature, comprising a set of 39 genes (listed below in Table 4), according to cancer type.

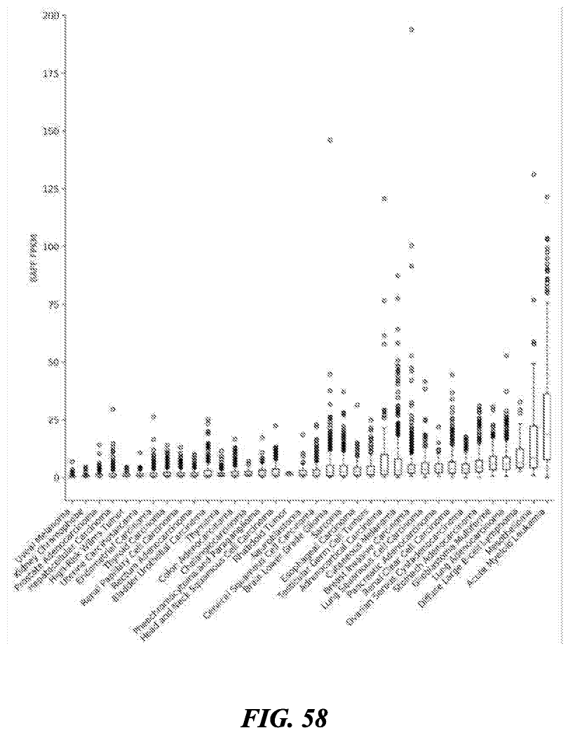

The Cancer Genome Atlas (TCGA) RNA-seq gene expression data was analyzed in 11,574 cancer samples. 11,574 HTSeq-FPKM (High Throughput Sequencing-Fragments Per Kilobase of transcript per Million mapped reads) files (i.e., 1 HTSeq-FPKM file per cancer sample) files representing an equivalent number of cancer samples were acquired from TCGA data release v9.0. Files were annotated with disease type based on the associated project annotations also acquired from TCGA release v.9.0. Sample data points for BAFF expression (TCGA identifier ENSG00000102524.10) were displayed as a boxplot with outliers and ordered left to right based on increasing median FPKM, as shown in FIG. 58. In addition, Table 6, below, provides number of samples analyzed based on disease types.

TABLE 6

| Disease type | Count |
|---|---|
| Neuroblastoma | 157 |
| Brain Lower Grade Glioma | 529 |
| Endometrial Carcinoma | 587 |
| Ovarian Serous Cystadenocarcinoma | 379 |
| Acute Myeloid Leukemia | 338 |
| Renal Papillary Cell Carcinoma | 321 |
| Colon Adenocarcinoma | 521 |
| Hepatocellular Carcinoma | 424 |
| Thyroid Carcinoma | 568 |
| Breast Invasive Carcinoma | 1222 |
| Renal Clear Cell Carcinoma | 611 |
| High-Risk Wilms Tumor | 132 |
| Bladder Urothelial Carcinoma | 433 |
| Prostate Adenocarcinoma | 551 |
| Lung Adenocarcinoma | 594 |
| Glioblastoma Multiforme | 174 |
| Cervical Squamous Cell Carcinoma | 309 |
| Head and Neck Squamous Cell Carcinoma | 546 |
| Stomach Adenocarcinoma | 407 |
| Cutaneous Melanoma | 472 |
| Esophageal Carcinoma | 173 |
| Rectum Adenocarcinoma | 177 |
| Cholangiocarcinoma | 45 |
| Pancreatic Adenocarcinoma | 182 |
| Lung Squamous Cell Carcinoma | 551 |
| Pheochromocytoma and Paraganglioma | 186 |
| Sarcoma | 265 |
| Testicular Germ Cell Tumors | 156 |
| Kidney Chromophobe | 89 |
| Mesothelioma | 86 |
| Thymoma | 121 |
| Uveal Melanoma | 80 |
| Adrenocortical Carcinoma | 79 |
| Uterine Carcinosarcoma | 56 |
| Diffuse Large B-cell Lymphoma | 48 |
| Rhabdoid Tumor | 5 |
| Total | 11574 |

11,574 HTSeq-FPKM (High Throughput Sequencing-Fragments Per Kilobase of transcript per Million mapped reads) files (i.e., 1HTSeq-FPKM file per cancer sample) were downloaded from TCGA data release v9.0. files. The HTSeq-FPKM files were annotated with cancer type based on the associated project annotations obtained from TCGA release v.9.0.

Samples representing the 36 cancer types in the TCGA RNA-Seq dataset were used for calculating an all-by-all correlation matrix. Rest of the samples were removed from the dataset. A Spearman's rank order correlation coefficient of the FPKM expression values for each gene pair, paired by sample, was calculated using the SciPy statistical package.

BAFF expression data was extracted using the Ensembl identifier ENSG00000102524.10.

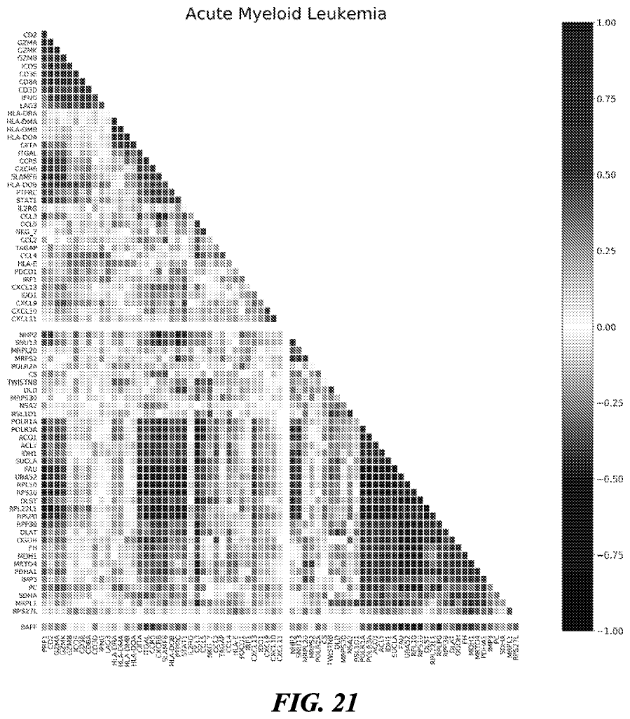

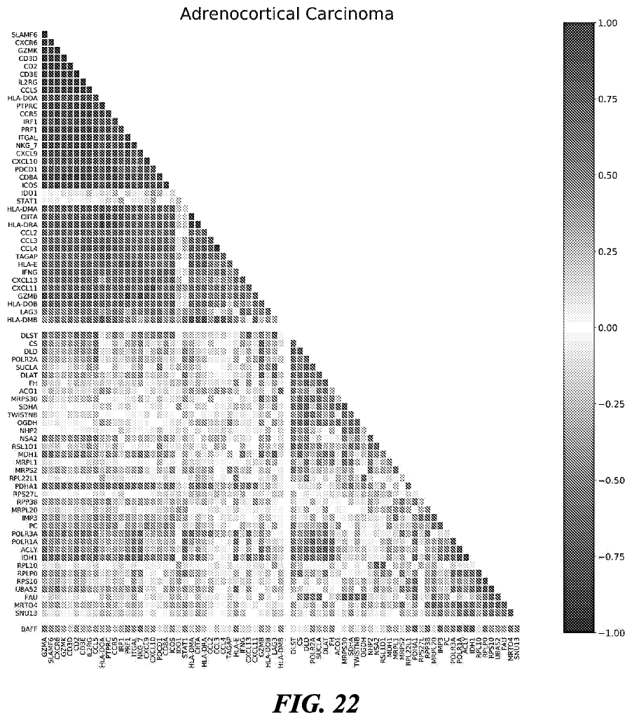

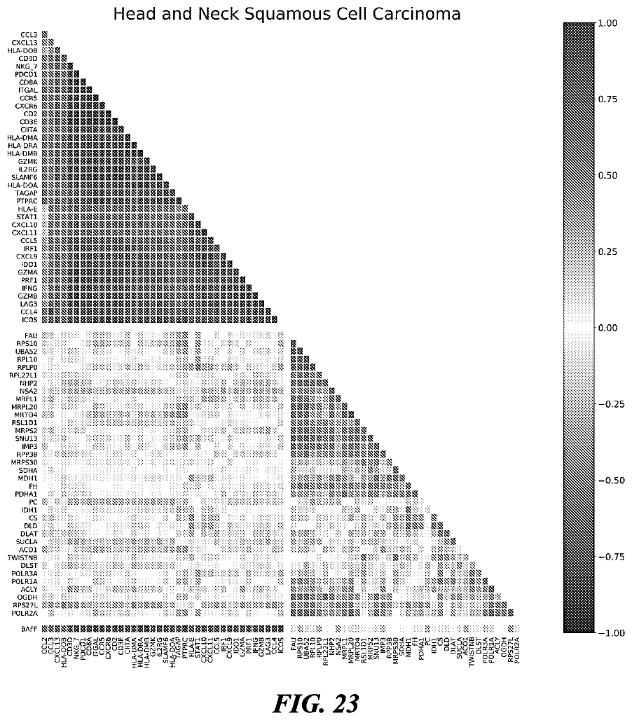

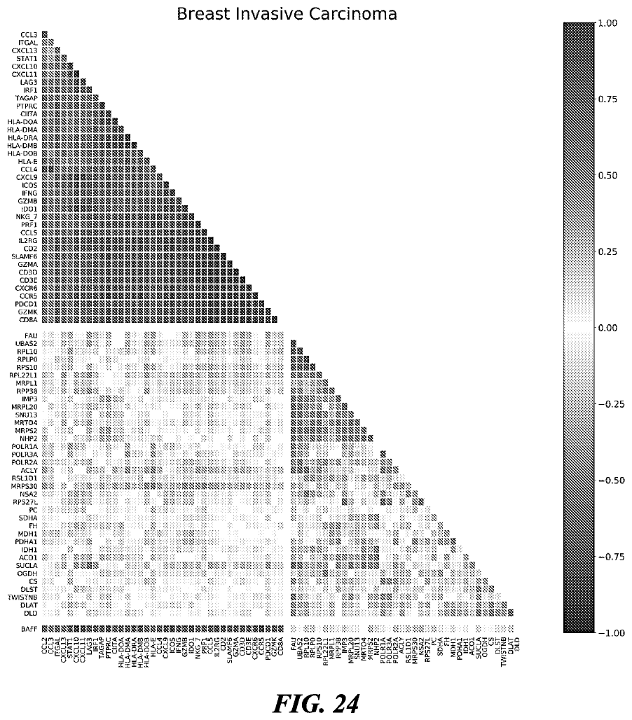

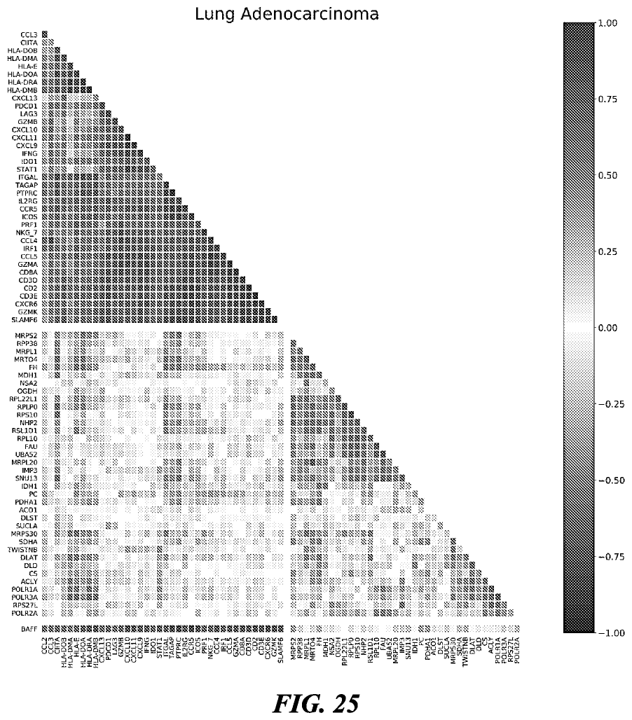

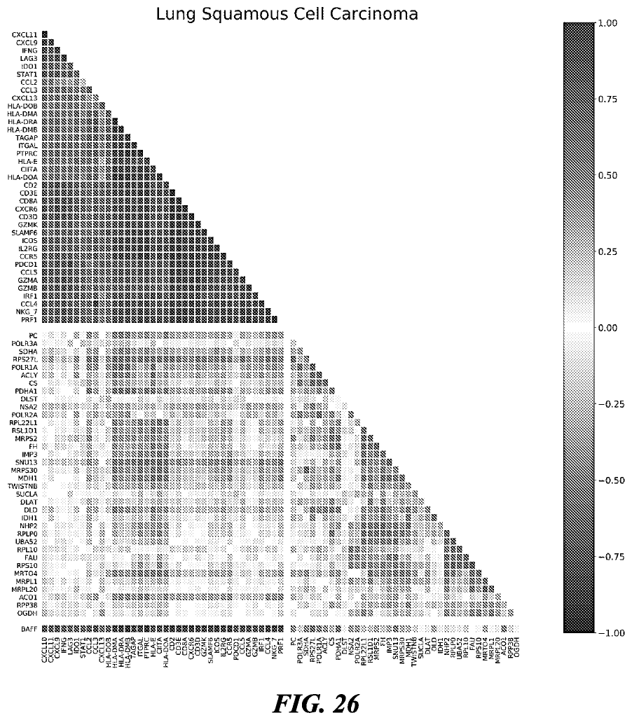

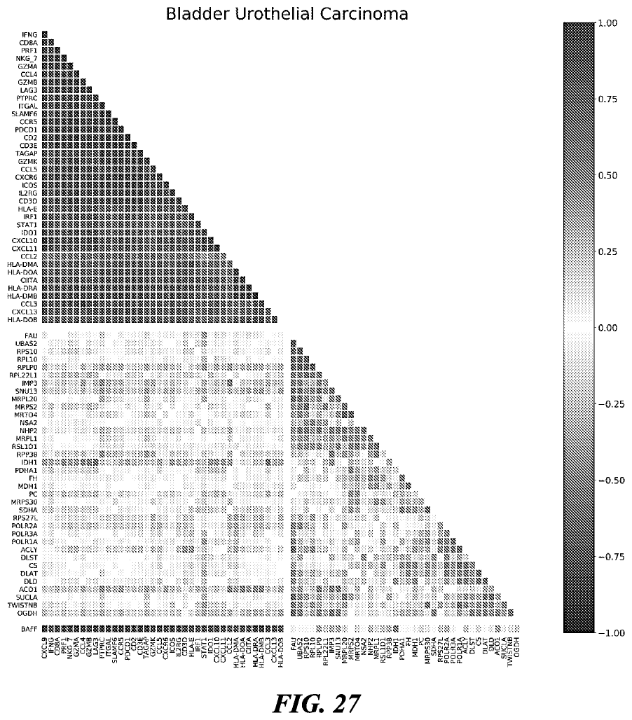

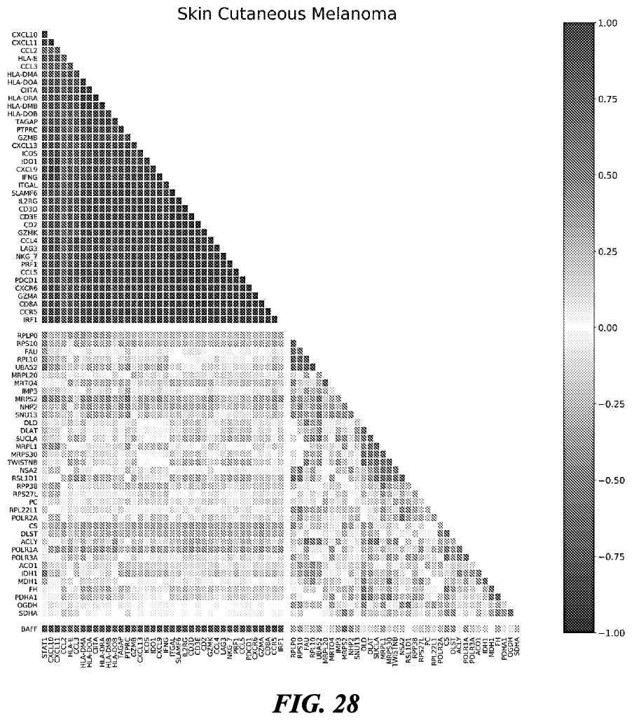

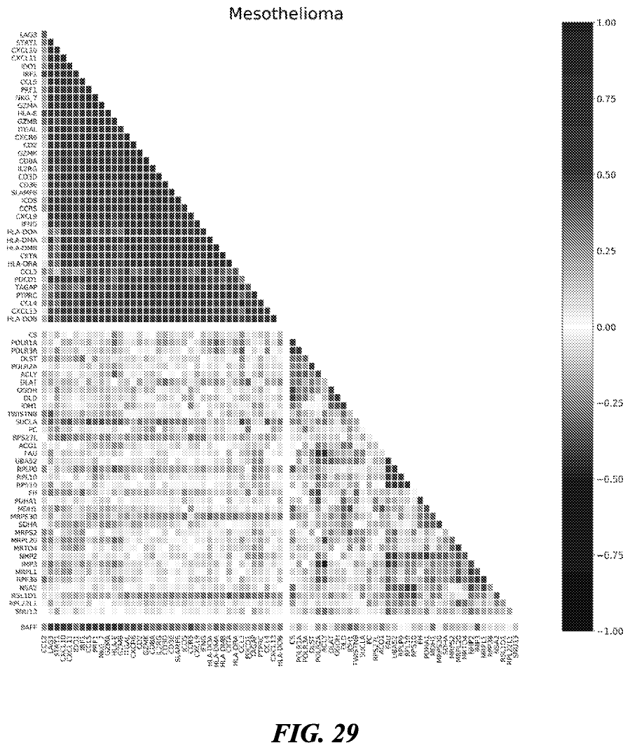

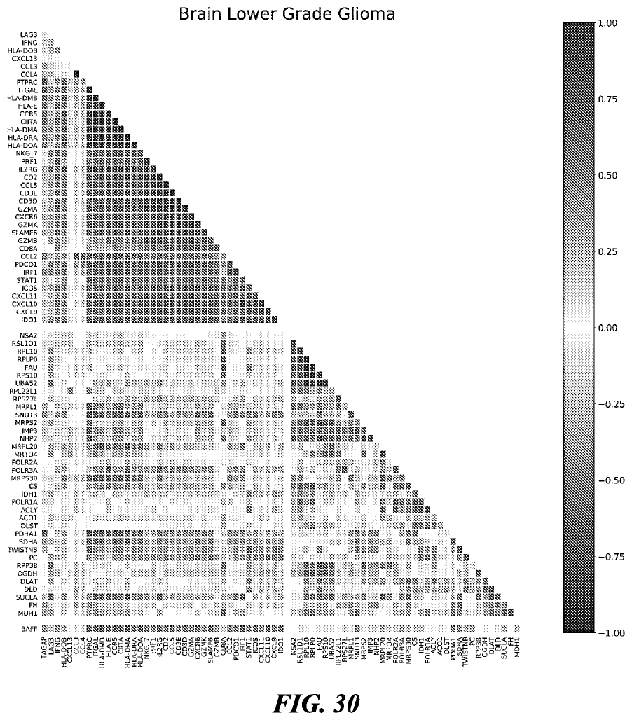

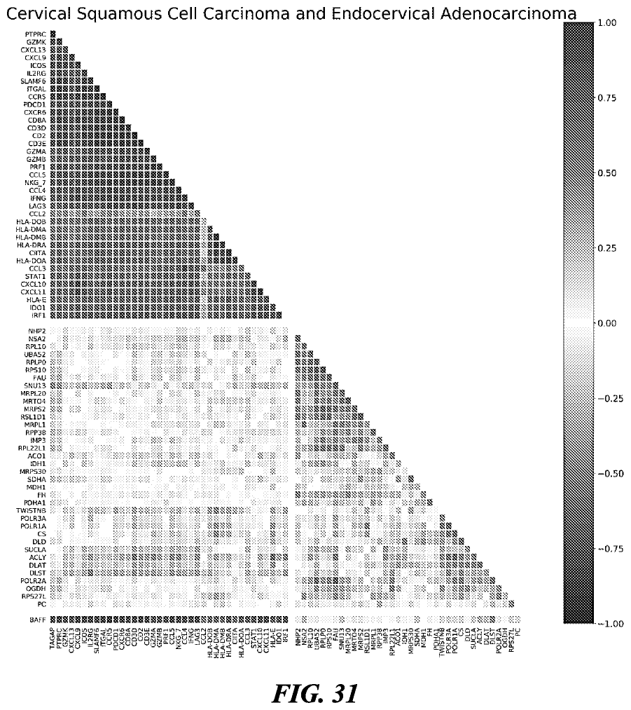

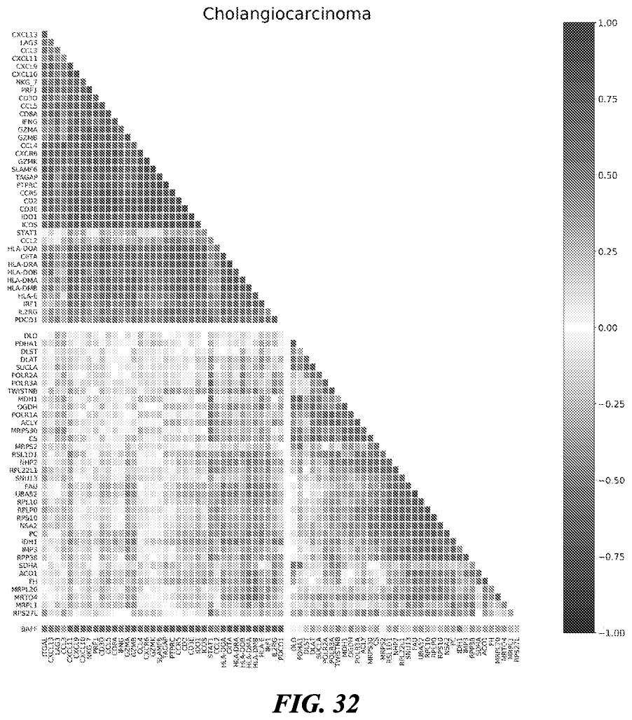

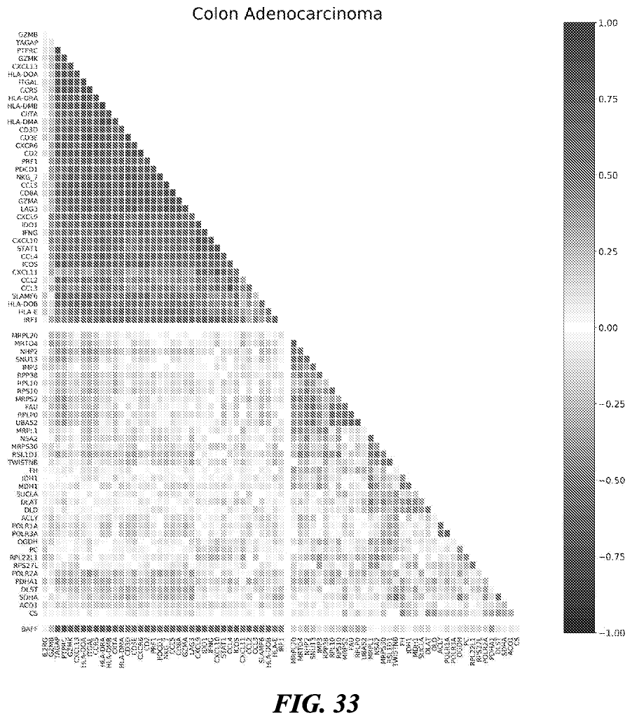

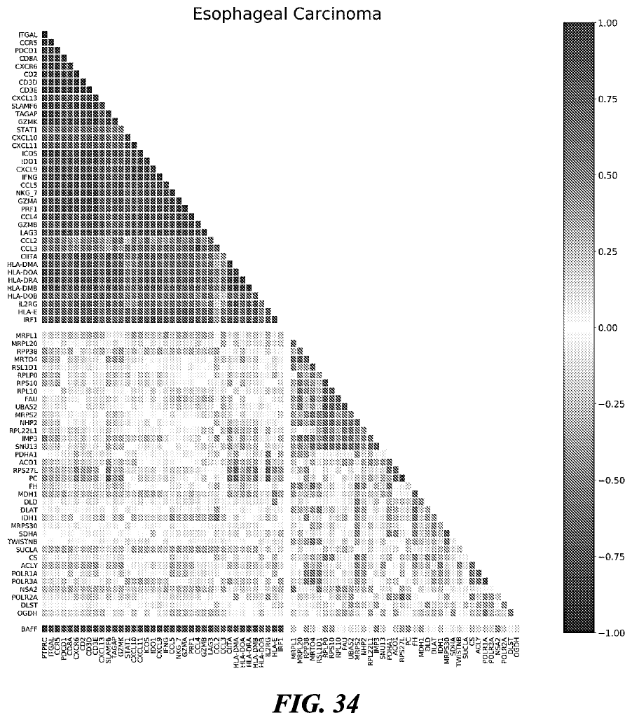

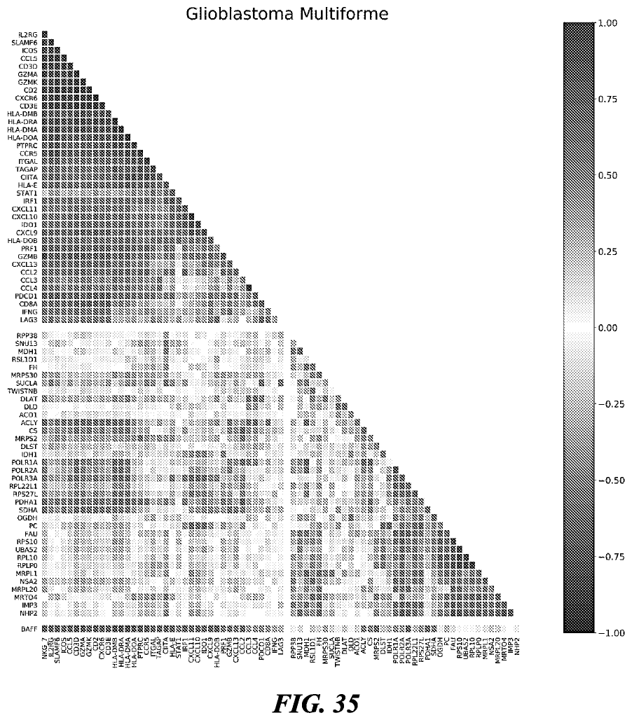

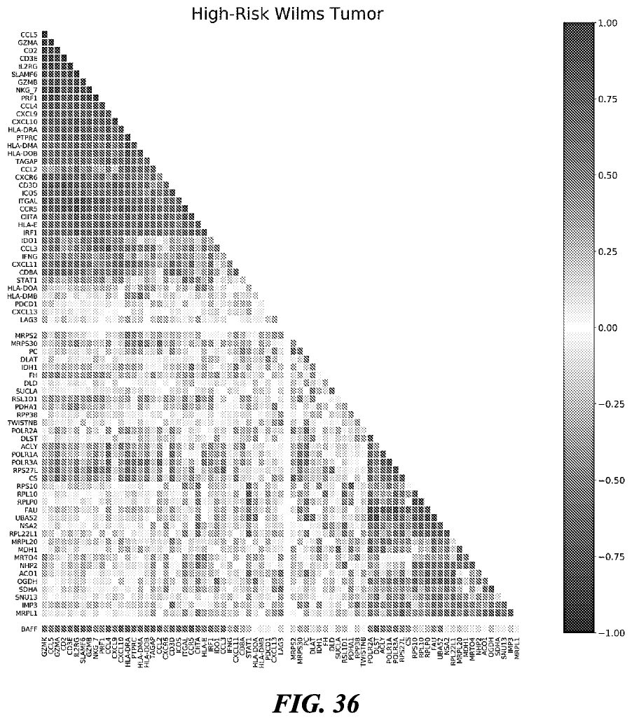

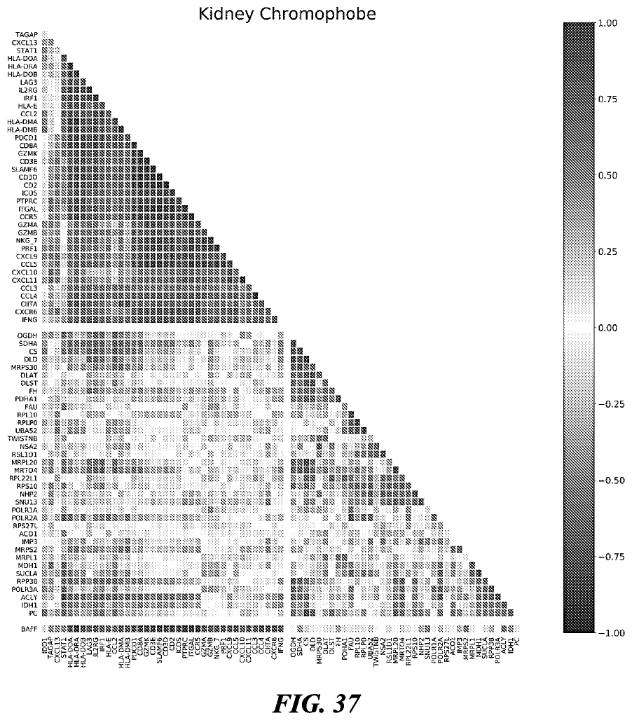

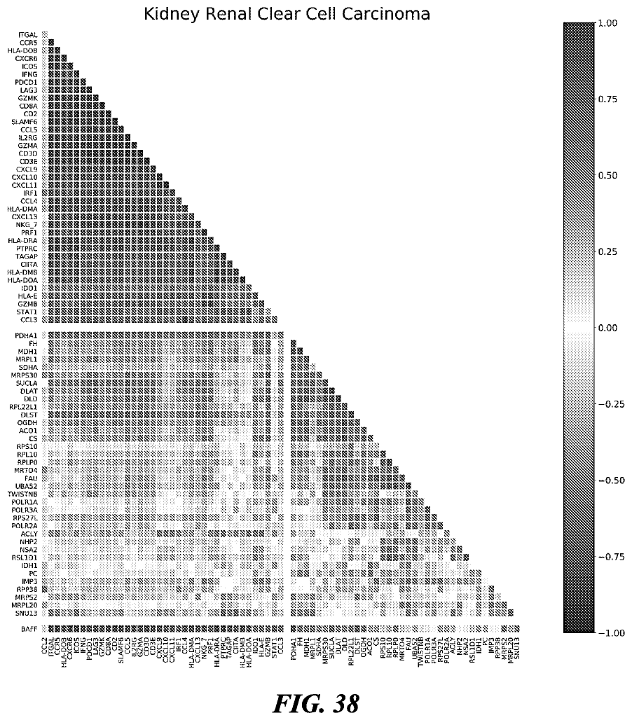

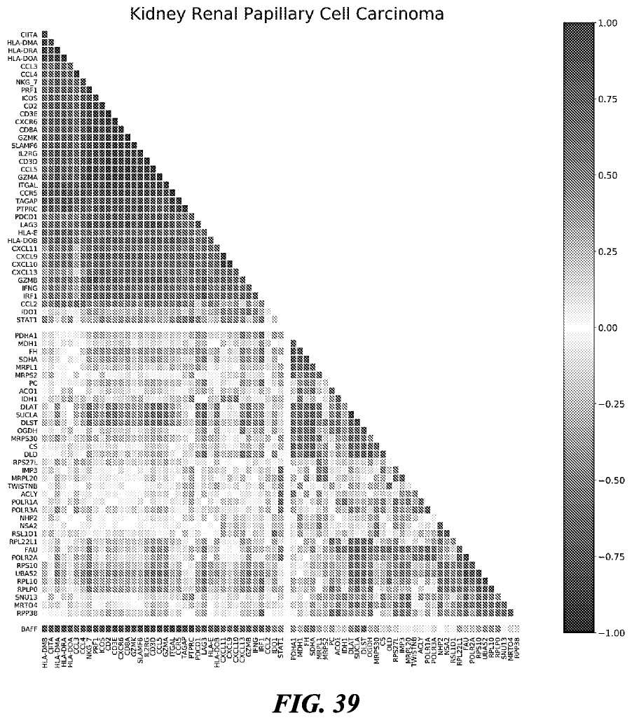

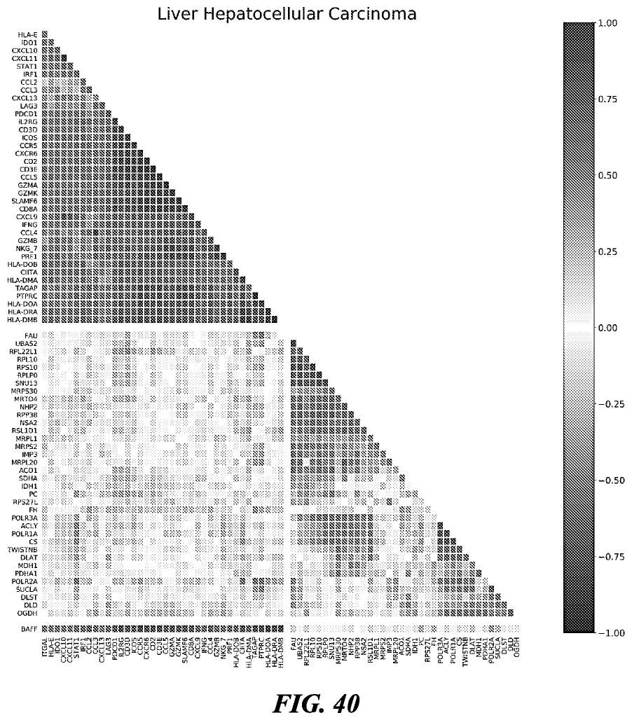

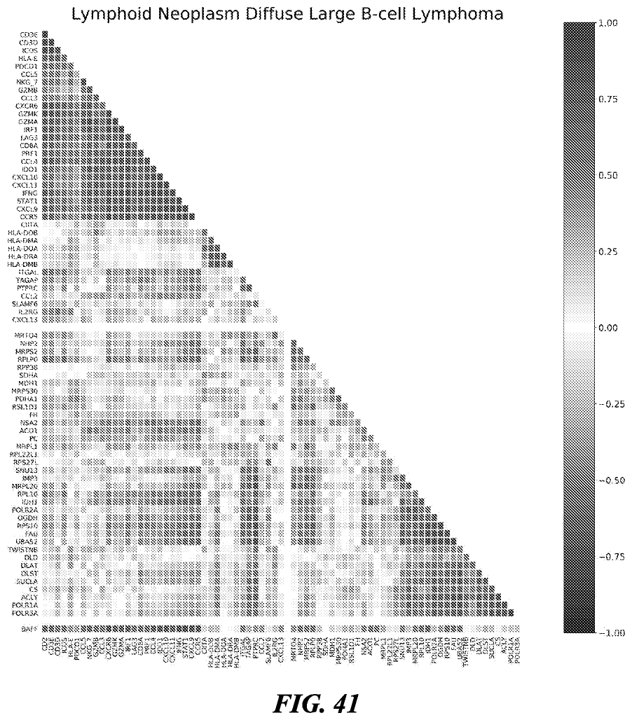

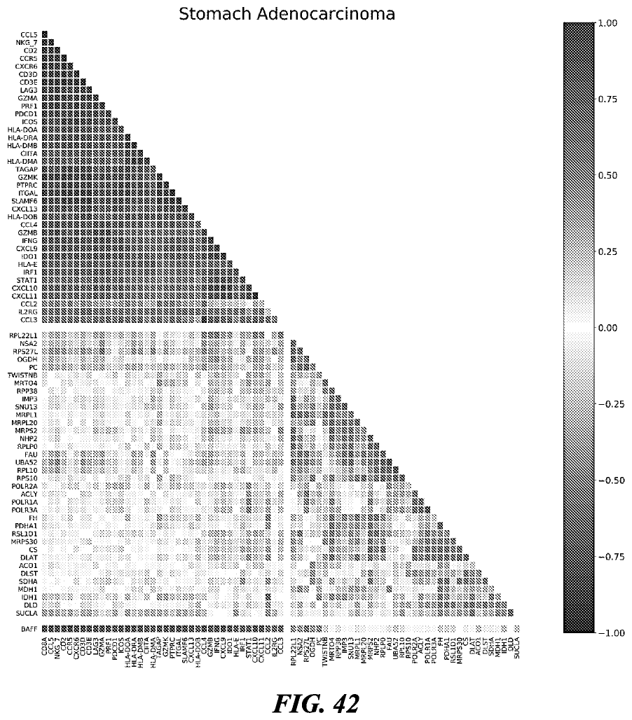

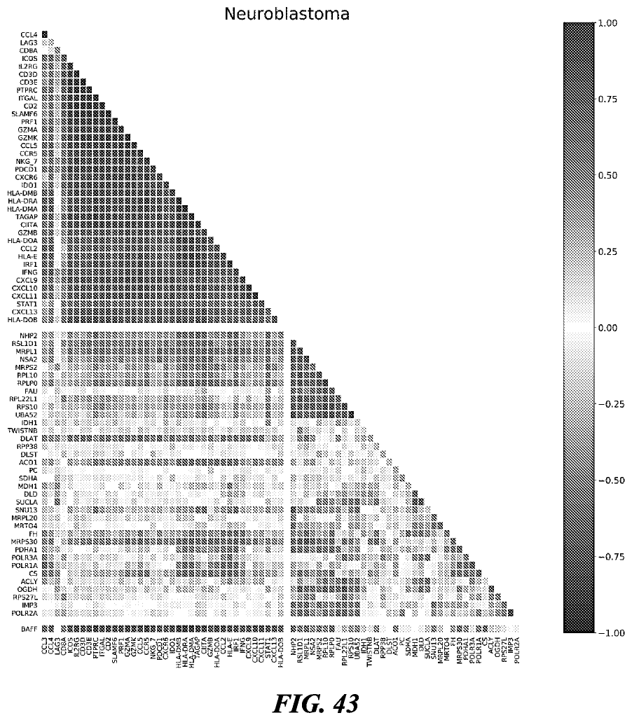

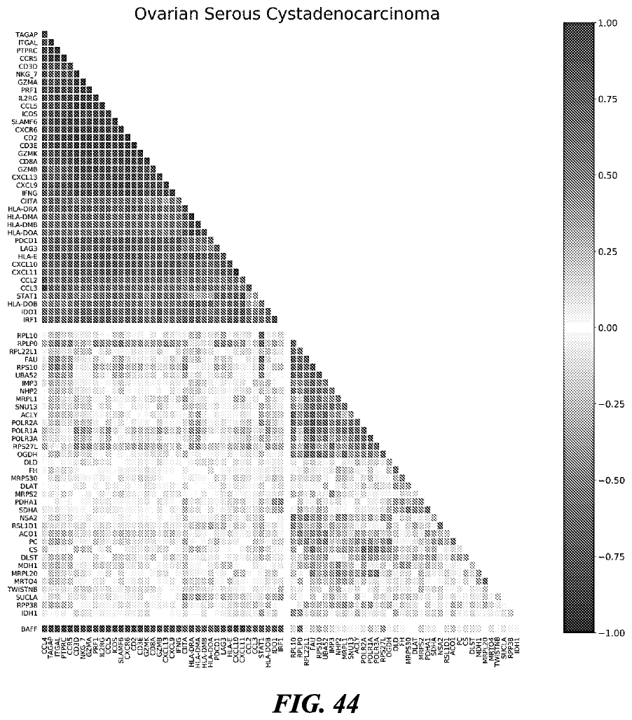

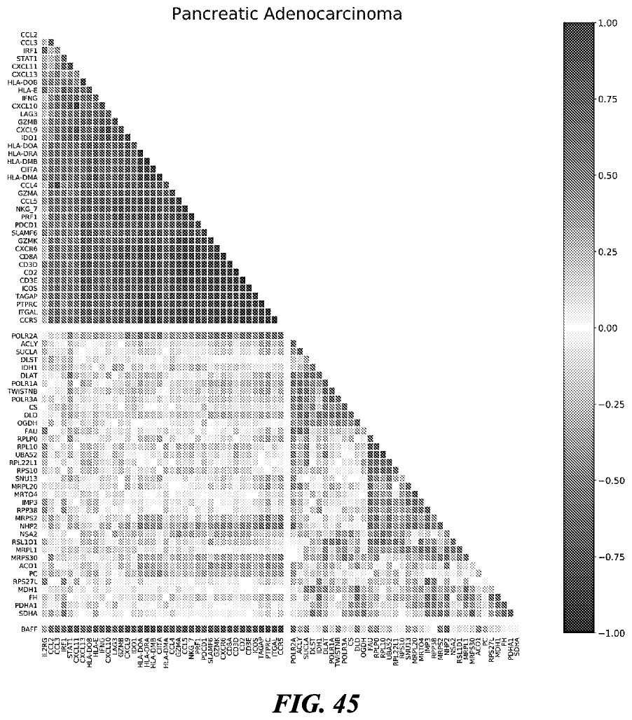

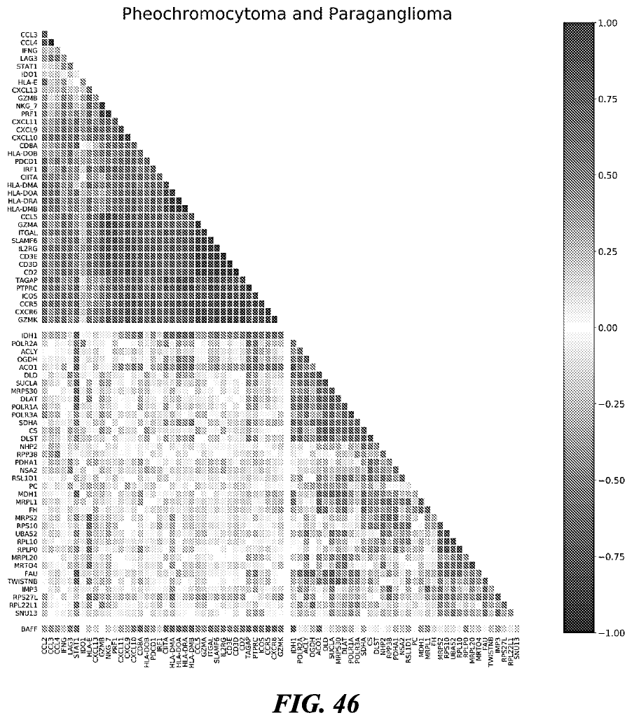

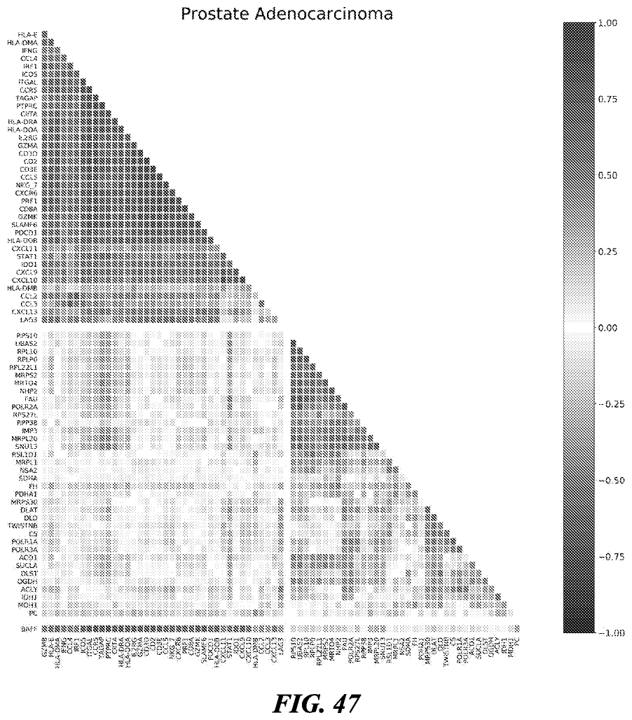

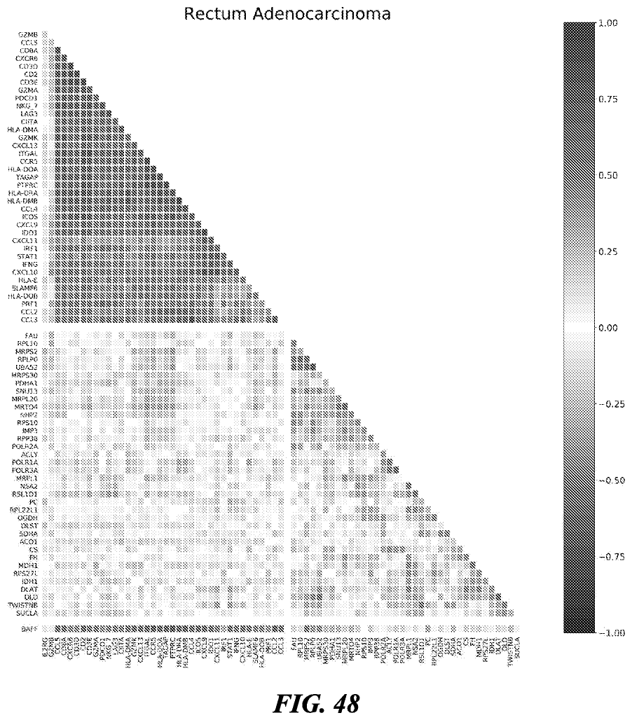

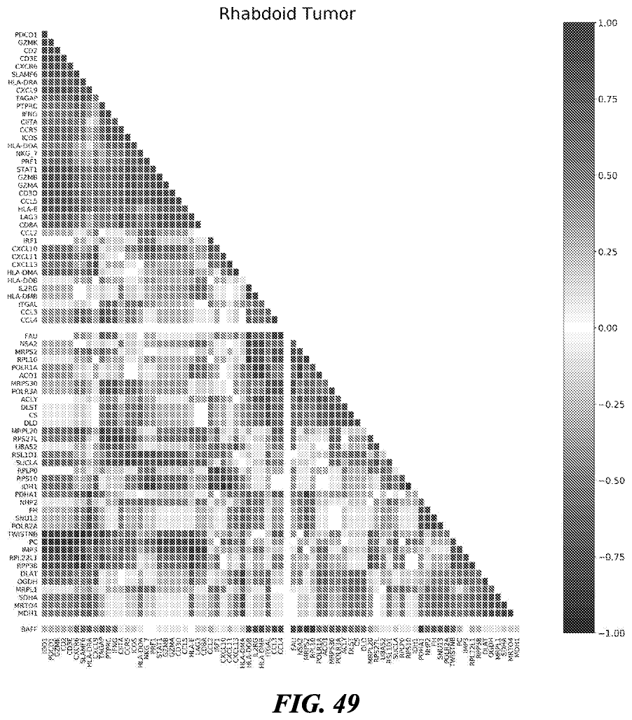

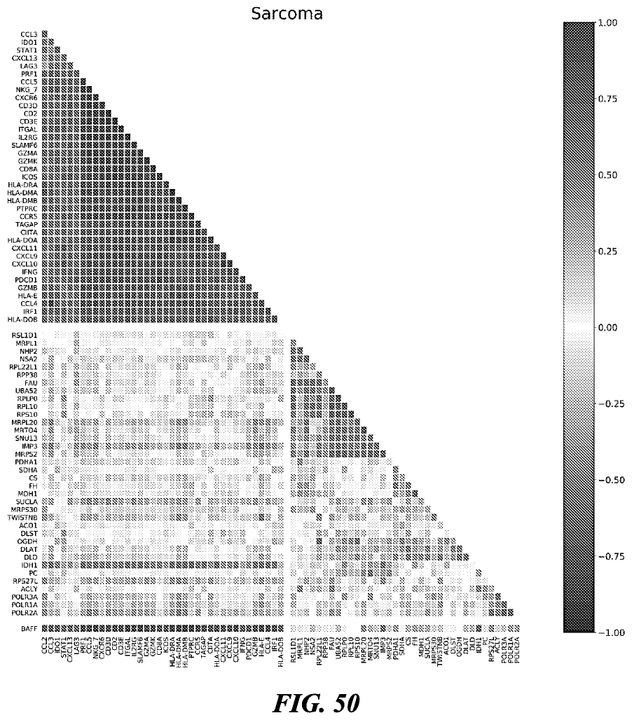

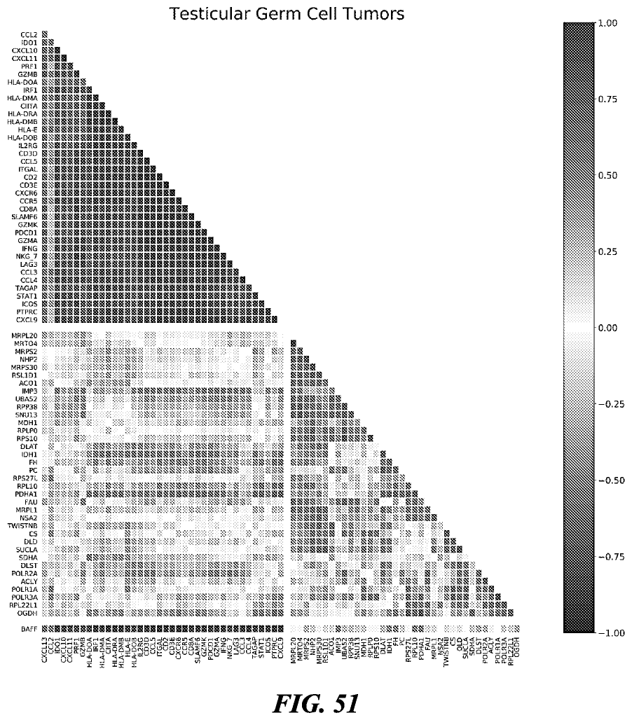

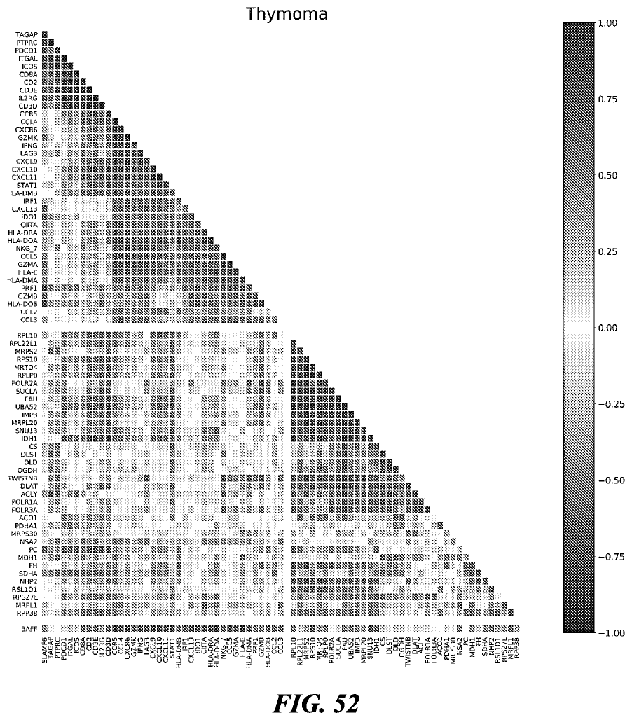

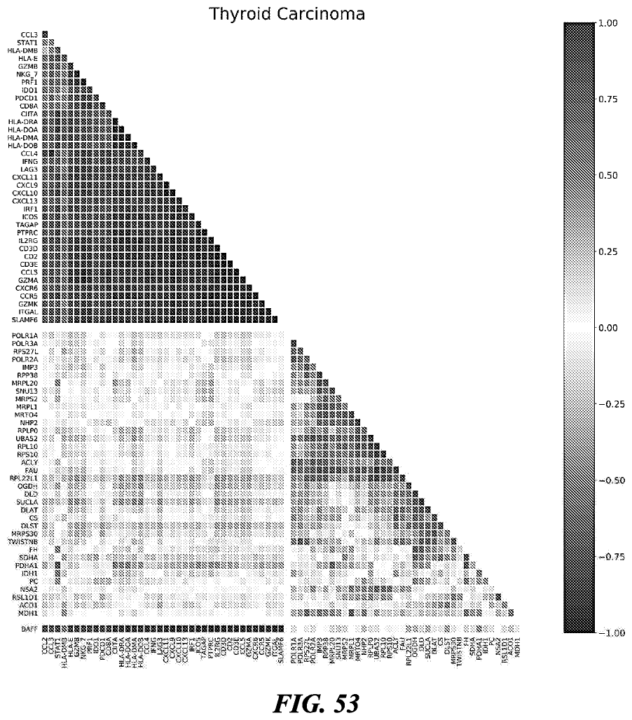

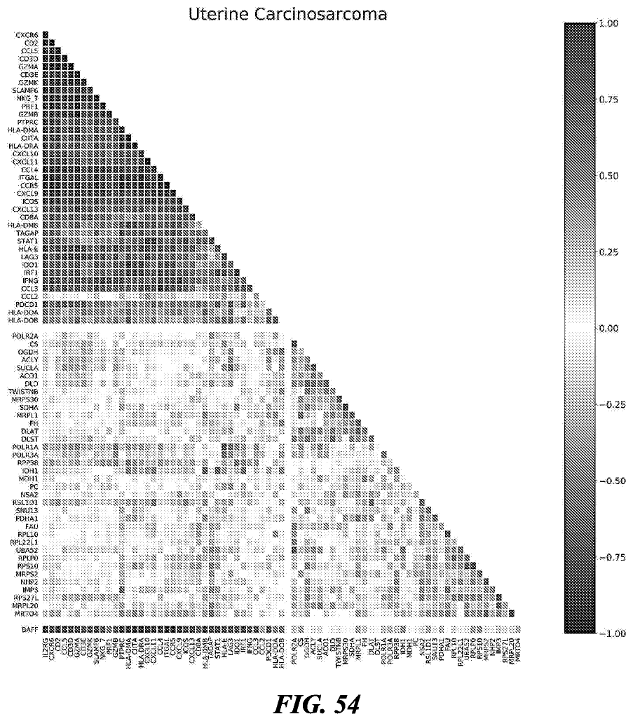

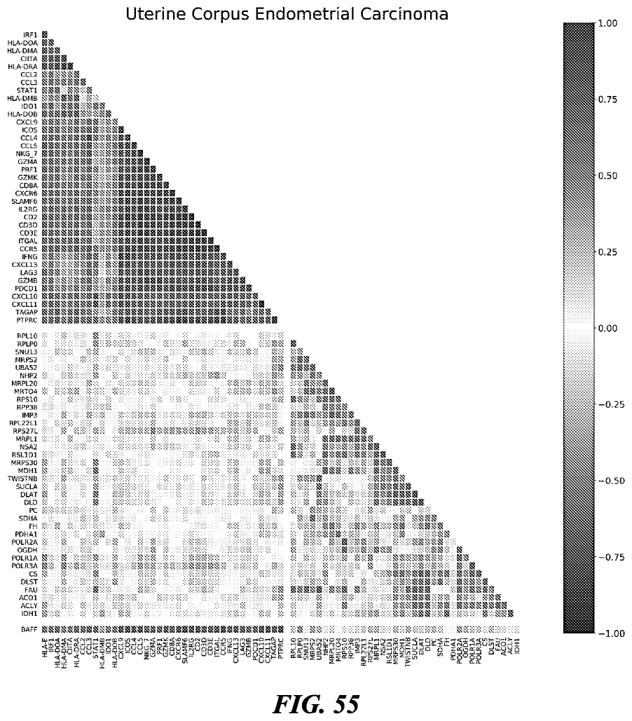

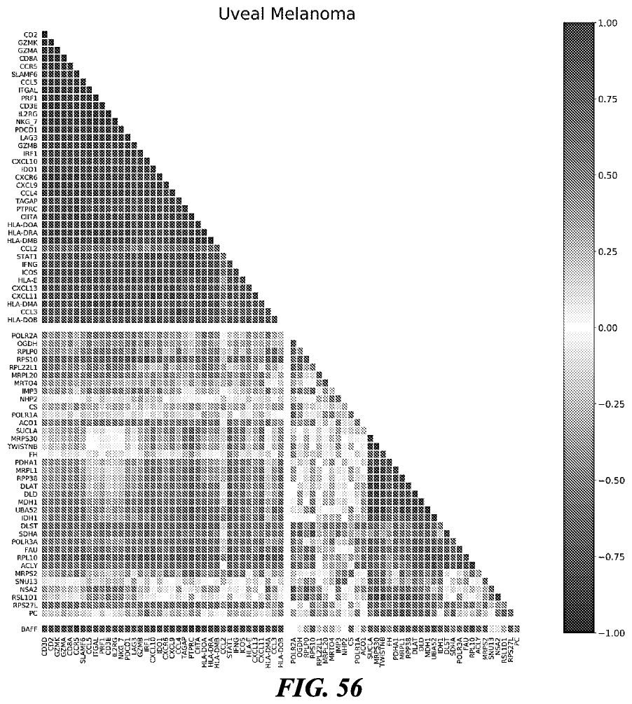

The correlation heatmap for the target correlation significance analysis was calculated as above. FIGS. 21-28 illustrate the heatmaps demonstrating the T-cell inflamed signature of several cancer types, such as, Acute Myeloid Leukemia (FIG. 21), Adrenocortical Carcinoma (FIG. 22), Head and Neck squamous cell carcinoma (FIG. 23), Breast Invasive Carcinoma (FIG. 24), Lung Adenocarcinoma (FIG. 25), Lung Squamous Cell Carcinoma (FIG. 26), Bladder Urothelial Carcinoma (FIG. 27), and Skin Cutaneous Melanoma (FIG. 28). In each of these heatmaps it can be seen that there is a significant correlation between the 39 genes that comprise the T-cell inflamed signature. The heatmaps depicted in FIG. 21 and FIG. 22 indicate that BAFF may not be strongly correlated with T-cell inflamed signature in Acute Myeloid Leukemia or Adrenocortical Carcinoma, respectively. The heatmap shown in FIG. 23 indicates that BAFF may be strongly correlated with T-cell inflamed signature in Head and Neck squamous cell carcinoma. The heatmap shown in FIG. 24 indicates that BAFF may have some correlation with T-cell inflamed signature in Breast Invasive Carcinoma. The heatmap shown in FIG. 25 indicates that BAFF may be strongly correlated with T-cell inflamed signature in Lung Adenocarcinoma. The heatmap shown in FIG. 26 indicates that BAFF may be strongly correlated with T-cell inflamed signature in Lung Squamous Cell Carcinoma. The heatmap shown in FIG. 27 indicates that BAFF may be strongly correlated with T-cell inflamed signature in Bladder Urothelial Carcinoma. The heatmap shown in FIG. 28 indicates that BAFF may be strongly correlated with T-cell inflamed signature in Skin Cutaneous Melanoma. The heatmap shown in FIG. 29 indicates that BAFF may have some correlation with T-cell inflamed signature in Mesothelioma. The heatmap shown in FIG. 30 indicates that BAFF may not be correlated with T-cell inflamed signature in Brain lower grade glioma. The heatmap shown in FIG. 31 indicates that BAFF may have some correlation with T-cell inflamed signature in Cervical squamous cell carcinoma and Endocervical adenocarcinoma. The heatmap shown in FIG. 32 indicates that BAFF may have some correlation with T-cell inflamed signature in Cholangiocarcinoma. The heatmap shown in FIG. 33 indicates that BAFF may be strongly correlated with T-cell inflamed signature in Colon adenocarcinoma. The heatmap shown in FIG. 34 indicates that BAFF may have some correlation with T-cell inflamed signature in Esophageal carcinoma. The heatmap shown in FIG. 35 indicates that BAFF may have some correlation with T-cell inflamed signature in Glioblastoma multiforme. The heatmap shown in FIG. 36 indicates that BAFF may not be correlated with T-cell inflamed signature in High-risk Wilms Tumor. The heatmap shown in FIG. 37 indicates that BAFF may have some correlation with T-cell inflamed signature in Kidney chromophobe. The heatmap shown in FIG. 38 indicates that BAFF may have some correlation with T-cell inflamed signature in Kidney renal clear cell carcinoma. The heatmap shown in FIG. 39 indicates that BAFF may have some correlation with T-cell inflamed signature in Kidney renal papillary cell carcinoma. The heatmap shown in FIG. 40 indicates that BAFF may have some correlation with T-cell inflamed signature in Liver hepatocellular carcinoma. The heatmap shown in FIG. 41 indicates that BAFF may not be correlated with T-cell inflamed signature in Lymphoid neoplasm diffuse large B-cell lymphoma. The heatmap shown in FIG. 42 indicates that BAFF may have some correlation with T-cell inflamed signature in Stomach adenocarcinoma. The heatmap shown in FIG. 43 indicates that BAFF may have some correlation with T-cell inflamed signature in neuroblastoma. The heatmap shown in FIG. 44 indicates that BAFF may have some correlation with T-cell inflamed signature in Ovarian serous cystadenocarcinoma. The heatmap shown in FIG. 45 indicates that BAFF may have some correlation with T-cell inflamed signature in Pancreatic adenocarcinoma. The heatmap shown in FIG. 46 indicates that BAFF may not be correlated with T-cell inflamed signature in Pheochromocytoma or Paraganglioma. The heatmap shown in FIG. 47 indicates that BAFF may have some correlation with T-cell inflamed signature in Prostate adenocarcinoma. The heatmap shown in FIG. 48 indicates that BAFF may have some correlation with T-cell inflamed signature in Rectum adenocarcinoma. The heatmap shown in FIG. 49 indicates that BAFF may not be correlated with T-cell inflamed signature in Rhabdoid tumor. The heatmap shown in FIG. 50 indicates that BAFF may have some correlation with T-cell inflamed signature in Sarcoma. The heatmap shown in FIG. 51 indicates that BAFF may have some correlation with T-cell inflamed signature in Testicular germ cell tumors. The heatmap shown in FIG. 52 indicates that BAFF may not be correlated with T-cell inflamed signature in Thymoma. The heatmap shown in FIG. 53 indicates that BAFF may be strongly correlated with T-cell inflamed signature in Thyroid carcinoma. The heatmap shown in FIG. 54 indicates that BAFF may have some correlation with T-cell inflamed signature in Uterine carcinosarcoma. The heatmap shown in FIG. 55 indicates that BAFF may have some correlation with T-cell inflamed signature in Uterine corpus endothelial carcinoma. The heatmap shown in FIG. 56 indicates that BAFF may be strongly correlated with T-cell inflamed signature in uveal melanoma.

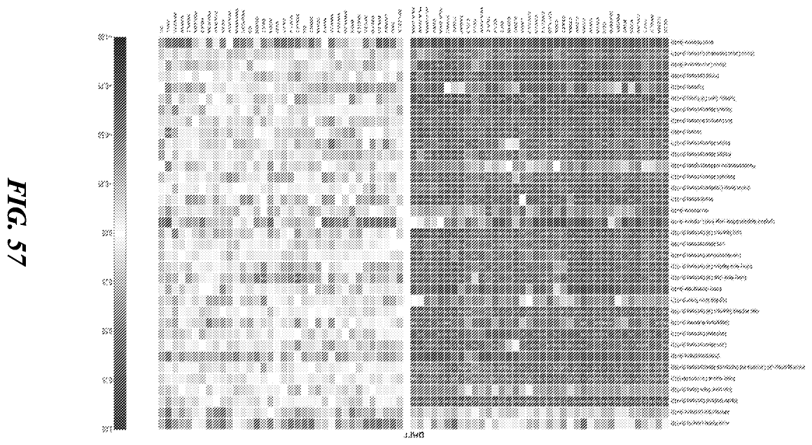

An aggregate heatmap for correlation between BAFF expression and the T-cell inflamed signature is shown in FIG. 57. BAFF expression was found to strongly correlate with the T-cell inflamed signature in a number of cancers, such as, cholangocarcinoma, lung squamous cell carcinoma.

The disease “Rhabdoid Tumor” was excluded due to the low number of samples available for analysis (N=5). Significance for each Spearman's Rho was calculated as follows. A model of random sample gene correlation was constructed for each disease by calculating Spearman's Rho for a set of randomly selected 10,000 pairs of genes. Genes with no measured expression in any sample were excluded from this random model. A mean and standard deviation of this background model was calculated for each disease and used to determine the required Z-score and corresponding significant Spearman's Rho values for a two-sided P-value test <0.01 corrected for multiple testing using the Bonferroni method. Spearman's rho values corresponding to those that are significant were displayed on the heatmap and those below significance were not displayed.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein may be employed in practicing the invention. It is intended that the following claims define the scope of the invention and that methods and structures within the scope of these claims and their equivalents be covered thereby.

TABLE 7
Sequences

| | | | | RefSeq |
|---|---|---|---|---|
| SEQ ID | | Amino Acid | | Genomic |
| NO: | Protein | Sequence | Exemplary mRNA sequence | Sequence |
| 36 | CCL2 | >tr\|J3KRT7\|J3KRT7_ | SEQ ID NO: 86 | SEQ ID NO: |
| | | HUMAN C-C motif | >NM_002982.3 Homo sapiens C-C motif chemokine ligand 2 (CCL2), mRNA | 124 |
| | | chemokine 2 | GAGGAACCGAGAGGCTGAGACTAACCCAGAAACATCCAATTCTCAAACTGAA | NG_012123.1 |
| | | OS = Homo sapiens | GCTCGCACTCTCGCCTCCAGCATGAAAGTCTCTGCCGCCCTTCTGTGCCTGCTG | |
| | | GN = CCL2 PE = 4 | CTCATAGCAGCCACCTTCATTCCCCAAGGGCTCGCTCAGCCAGATGCAATCAA | |
| | | SV = 1 | TGCCCCAGTCACCTGCTGTTATAACTTCACCAATAGGAAGATCTCAGTGCAGA | |
| | | MKVSAALLCLLLI | GGCTCGCGAGCTATAGAAGAATCACCAGCAGCAAGTGTCCCAAAGAAGCTGT | |
| | | AATFIPQGLAQPD | GATCTTCAAGACCATTGTGGCCAAGGAGATCTGTGCTGACCCCAAGCAGAAGT | |
| | | AINAPVTCCYNFT | GGGTTCAGGATTCCATGGACCACCTGGACAAGCAAACCCAAACTCCGAAGAC | |
| | | NRKISVQRLASYR | TTGAACACTCACTCCACAACCCAAGAATCTGCAGCTAACTTATTTTCCCCTAG | |
| | | RITSSKCPKEAVM | CTTTCCCCAGACACCCTGTTTTATTTTATTATAATGAATTTTGTTTGTTGATGTG | |
| | | | AAACATTATGCCTTAAGTAATGTTAATTCTTATTTAAGTTATTGATGTTTTAAG | |
| | | | TTTATCTTTCATGGTACTAGTGTTTTTTAGATACAGAGACTTGGGGAAATTGCT | |
| | | | TTTCCTCTTGAACCACAGTTCTACCCCTGGGATGTTTTGAGGGTCTTTGCAAGA | |
| | | | ATCATTAATACAAAGAATTTTTYTTAACATTCCAATGCATTGCTAAAATATTAT | |
| | | | TGTGGAAATGAATATTTTGTAACTATTACACCAAATAAATATATTTTTGTACA | |
| | | | AAAAAAAAAAAAAA | |
| 37 | CCL3 | >NP_002974.1 C-C | SEQ ID NO: 87 | SEQ ID NO: |
| | | motif chemokine 3 | >NM_002983.2 Homo sapiens C-C motif chemokine ligand 3 (CCL3), mRNA | 125 |
| | | precursor [Homo | AGCTGGTTTCAGACTTCAGAAGGACACGGGCAGCAGACAGTGGTCAGTCCTTT | NG_027730.1 |
| | | sapiens] | CTTGGCTCTGCTGACACTCGAGCCCACATTCCGTCACCTGCTCAGAATCATGC | |
| | | MQVSTAALAVLL | AGGTCTCCACTGCTGCCCTTGCTGTCCTCCTCTGCACCATGGCTCTCTGCAACC | |
| | | CTMALCNQFSASL | AGTTCTCTGCATCACTTGCTGCTGACACGCCGACCGCCTGCTGCTTCAGCTACA | |
| | | AADTPTACCFSYT | CCTCCCGGCAGATTCCACAGAATTTCATAGCTGACTACTTTGAGACGAGCAGC | |
| | | SRQIPQNFIADYFE | CAGTGCTCCAAGCCCGGTGTCATCTTCCTAACCAAGCGAAGCCGGCAGGTCTG | |
| | | TSSQCSKPGVIFLT | TGCTGACCCCAGTGAGGAGTGGGTCCAGAAATATGTCAGCGACCTGGAGCTG | |
| | | KRSRQVCADPSEE | AGTGCCTGAGGGGTCCAGAAGCTTCGAGGCCCAGCGACCTCGGTGGGCCCAG | |
| | | WVQKYVSDLELS | TGGGGAGGAGCAGGAGCCTGAGCCTTGGGAACATGCGTGTGACCTCCACAGC | |
| | | A | TACCTCTTCTATGGACTGGTTGTTGCCAAACAGCCACACTGTGGGACTCTTCTT | |
| | | | AACTTAAATTTTAATTTATTTATACTATTTAGTTTTTGTAATTTATTTTCGATTT | |
| | | | CACAGTGTGTTTGTGATTGTTTGCTCTGAGAGTTCCCCTGTCCCCTCCCCCTTC | |
| | | | CCTCACACCGCGTCTGGTGACAACCGAGTGGCTGTCATCAGCCTGTGTAGGCA | |
| | | | GTCATGGCACCAAAGCCACCAGACTGACAAATGTGTATCGGATGCTTTTGTTC | |
| | | | AGGGCTGTGATCGGCCTGGGGAAATAATAAAGATGCTCTTTTAAAAGGTAAA | |
| | | | AAAAAAAAAAAAAAAA | |
| 38 | ITGAL | >NP_002200.2 | SEQ ID NO: 88 | |
| | | integrin alpha-L | >NM_002209.2 Homo sapiens integrin subunit alpha L (ITGAL), transcript variant 1, | |
| | | isoform a precursor | mRNA | |
| | | [Homo sapiens] | ACACCTCCCTCCCCGCCTGCCAGTGTCACCAGCCTGTTGCCTCTGTGAGAAAG | |
| | | MKDSCITVMAMA | TACCACTGTAAGAGGCCAAAGGGCATGATCATTTTCCTCTTTCACCCTGTCTA | |
| | | LLSGFFFFAPASSY | GGTTGCCAGCAAATCCCACGGGCCTCCTGACGCTGCCCCTGGGGCCACAGGTC | |
| | | NLDVRGARSFSPP | CCTCGAGTGCTGGAAGGATGAAGGATTCCTGCATCACTGTGATGGCCATGGCG | |
| | | RAGRHFGYRVLQ | CTGCTGTCTGGGTTCTTTTTCTTCGCGCCGGCCTCGAGCTACAACCTGGACGTG | |
| | | VGNGVIVGAPGEG | CGGGGCGCGCGGAGCTTCTCCCCACCGCGCGCCGGGAGGCACTTTGGATACC | |
| | | NSTGSLYQCQSGT | GCGTCCTGCAGGTCGGAAACGGGGTCATCGTGGGAGCTCCAGGGGAGGGGAA | |
| | | GHCLPVTLRGSNY | CAGCACAGGAAGCCTCTATCAGTGCCAGTCGGGCACAGGACACTGCCTGCCA | |
| | | TSKYLGMTLATDP | GTCACCCTGAGAGGTTCCAACTATACCTCCAAGTACTTGGGAATGACCTTGGC | |
| | | TDGSILACDPGLS | AACAGACCCCACAGATGGAAGCATTTTGGCCTGTGACCCTGGGCTGTCTCGAA | |
| | | RTCDQNTYLSGLC | CGTGTGACCAGAACACCTATCTGAGTGGCCTGTGTTACCTCTTCCGCCAGAAT | |
| | | YLFRQNLQGPML | CTGCAGGGTCCCATGCTGCAGGGGCGCCCTGGTTTTCAGGAATGTATCAAGGG | |
| | | QGRPGFQECIKGN | CAACGTAGACCTGGTATTTCTGTTTGATGGTTCGATGAGCTTGCAGCCAGATG | |
| | | VDLVFLFDGSMSL | AATTTCAGAAAATTCTGGACTTCATGAAGGATGTGATGAAGAAACTCAGCAA | |
| | | QPDEFQKILDFMK | CACTTCGTACCAGTTTGCTGCTGTTCAGTTTTCCACAAGCTACAAAACAGAATT | |
| | | DVMKKLSNTSYQ | TGATTTCTCAGATTATGTTAAACGGAAGGACCCTGATGCTCTGCTGAAGCATG | |
| | | VFAAVQFSTSYKTE | TAAAGCACATGTTGCTGTTGACCAATACCTTTGGTGCCATCAATTATGTCGCG | |
| | | FDFSDYVKRKDPD | ACAGAGGTGTTCCGGGAGGAGCTGGGGGCCCGGCCAGATGCCACCAAAGTGC | |
| | | ALLKHVKHMLLL | TTATCATCATCACGGATGGGGAGGCCACTGACAGTGGCAACATCGATGCGGC | |
| | | TNTFGAINYVATE | CAAAGACATCATCCGCTACATCATCGGGATTGGAAAGCATTTTCAGACCAAGG | |
| | | VFREELGARPDAT | AGAGTCAGGAGACCCTCCACAAATTTGCATCAAAACCCGCGAGCGAGTTTGT | |
| | | VKVLIIITDGEATDS | GAAAATTCTGGACACATTTGAGAAGCTGAAAGATCTATTCACTGAGCTGCAGA | |
| | | GNIDAAKDIIRYII | AGAAGATCTATGTCATTGAGGGCACAAGCAAACAGGACCTGACTTCCTTCAAC | |
| | | GIGKHFQTKESQE | ATGGAGCTGTCCTCCAGCGGCATCAGTGCTGACCTCAGCAGGGGCCATGCAGT | |
| | | TLHKFASKPASEF | CGTGGGGGCAGTAGGAGCCAAGGACTGGGCTGGGGGCTTTCTTGACCTGAAG | |
| | | VKILDITEKLKDL | GCAGACCTGCAGGATGACACATTTATTGGGAATGAACCATTGACACCAGAAG | |
| | | FTELQKKIYVIEGT | TGAGAGCAGGCTATTTGGGTTACACCGTGACCTGGCTGCCCTCCCGGCAAAAG | |
| | | SKQDLTSFNMELS | ACTTCGTTGCTGGCCTCGGGAGCCCCTCGATACCAGCACATGGGCCGAGTGCT | |
| | | SSGISADLSRGHA | GCTGTTCCAAGAGCCACAGGGCGGAGGACACTGGAGCCAGGTCCAGACAATC | |
| | | VVGAVGAKDWA | CATGGGACCCAGATTGGCTCTTATTTCGGTGGGGAGCTGTGTGGCGTCGACGT | |
| | | GGFLDLKADLQD | GGACCAAGATGGGGAGACAGAGCTGCTGCTGATTGGTGCCCCACTGTTCTATG | |
| | | DTFIGNEPLTPEVR | GGGAGCAGAGAGGAGGCCGGGTGTTTATCTACCAGAGAAGACAGTTGGGGTT | |
| | | AGYLGYTVTWLP | TGAAGAAGTCTCAGAGCTGCAGGGGGACCCCGGCTACCCACTCGGGCGGTTT | |
| | | SRQKTSLLASGAP | GGAGAAGCCATCACTGCTCTGACAGACATCAACGGCGATGGGCTGGTAGACG | |
| | | RYQHMGRVLLFQ | TGGCTGTGGGGGCCCCTCTGGAGGAGCAGGGGGCTGTGTACATCTTCAATGGG | |
| | | EPQGGGHWSQVQ | AGGCACGGGGGGCTTAGTCCCCAGCCAAGTCAGCGGATAGAAGGGACCCAAG | |
| | | TIFIGTQIGSYEGGE | TGCTCTCAGGAATTCAGTGGTTTGGACGCTCCATCCATGGGGTGAAGGACCTT | |
| | | LCGVDVDQDGET | GAAGGGGATGGCTTGGCAGATGTGGCTGTGGGGGCTGAGAGCCAGATGATCG | |
| | | ELLLIGAPLFYGEQ | TGCTGAGCTCCCGGCCCGTGGTGGATATGGTCACCCTGATGTCCTTCTCTCCAG | |
| | | RGGRVFIYQRRQL | CTGAGATCCCAGTGCATGAAGTGGAGTGCTCCTATTCAACCAGTAACAAGATG | |
| | | GFEEVSELQGDPG | AAAGAAGGAGTTAATATCACAATCTGTTTCCAGATCAAGTCTCTCATCCCCCA | |
| | | YPLGRFGEAITAL | GTTCCAAGGCCGCCTGGTTGCCAATCTCACTTACACTCTGCAGCTGGATGGCC | |
| | | TDINGDGLVDVA | ACCGGACCAGAAGACGGGGGTTGTTCCCAGGAGGGAGACATGAACTCAGAAG | |
| | | VGAPLEEQGAVYI | GAATATAGCTGTCACCACCAGCATGTCATGCACTGACTTCTCATTTCATTTCCC | |
| | | FNGRHGGLSPQPS | GGTATGTGTTCAAGACCTCATCTCCCCCATCAATGTTTCCCTGAATTTCTCTCT | |
| | | QRIEGTQVLSGIQ | TTGGGAGGAGGAAGGGACACCGAGGGACCAAAGGGCGCAGGGCAAGGACAT | |
| | | WFGRSIHGVKDLE | ACCGCCCATCCTGAGACCCTCCCTGCACTCGGAAACCTGGGAGATCCCTTTTG | |
| | | GDGLADVAVGAE | AGAAGAACTGTGGGGAGGACAAGAAGTGTGAGGCAAACTTGAGAGTGTCCTT | |
| | | SQMIVLSSRPVVD | CTCTCCTGCAAGATCCAGAGCCCTGCGTCTAACTGCTTTTGCCAGCCTCTCTGT | |
| | | MVTLMSFSPAEIP | GGAGCTGAGCCTGAGTAACTTGGAAGAAGATGCTTACTGGGTCCAGCTGGAC | |
| | | VHEVECSYSTSNK | CTGCACTTCCCCCCGGGACTCTCCTTCCGCAAGGTGGAGATGCTGAAGCCCCA | |
| | | MKEGVNITICFQIK | TAGCCAGATACCTGTGAGCTGCGAGGAGCTTCCTGAAGAGTCCAGGCTTCTGT | |
| | | SLIPQFQGRLVAN | CCAGGGCATTATCTTGCAATGTGAGCTCTCCCATCTTCAAAGCAGGCCACTCG | |
| | | LTYTLQLDGHRTR | GTTGCTCTGCAGATGATGTTTAATACACTGGTAAACAGCTCCTGGGGGGACTC | |
| | | RRGLFPGGRHELR | GGTTGAATTGCACGCCAATGTGACCTGTAACAATGAGGACTCAGACCTCCTGG | |
| | | RNIAVTTSMSCTD | AGGACAACTCAGCCACTACCATCATCCCCATCCTGTACCCCATCAACATCCTC | |
| | | FSFHFPVCVQDLIS | ATCCAGGACCAAGAAGACTCCACACTCTATGTCAGTTTCACCCCCAAAGGCCC | |
| | | PINVSLNFSLWEEE | CAAGATCCACCAAGTCAAGCACATGTACCAGGTGAGGATCCAGCCTTCCATCC | |
| | | GTPRDQRAQGKDI | ACGACCACAACATACCCACCCTGGAGGCTGTGGTTGGGGTGCCACAGCCTCCC | |
| | | PPILRPSLHSETWE | AGCGAGGGGCCCATCACACACCAGTGGAGCGTGCAGATGGAGCCTCCCGTGC | |
| | | IPFEKNCGEDKKC | CCTGCCACTATGAGGATCTGGAGAGGCTCCCGGATGCAGCTGAGCCTTGTCTC | |
| | | EANLRVSFSPARS | CCCGGAGCCCTGTTCCGCTGCCCTGTTGTCTTCAGGCAGGAGATCCTCGTCCA | |
| | | RALRLTAFASLSV | AGTGATCGGGACTCTGGAGCTGGTGGGAGAGATCGAGGCCTCTTCCATGTTCA | |
| | | ELSLSNLEEDAYW | GCCTCTGCAGCTCCCTCTCCATCTCCTTCAACAGCAGCAAGCATTTCCACCTCT | |
| | | VQLDLHFPPGLSF | ATGGCAGCAACGCCTCCCTGGCCCAGGTTGTCATGAAGGTTGACGTGGTGTAT | |
| | | RKVEMLKPHSQIP | GAGAAGCAGATGCTCTACCTCTACGTGCTGAGCGGCATCGGGGGGCTGCTGCT | |
| | | VSCEELPEESRLLS | GCTGCTGCTCATTTTCATAGTGCTGTACAAGGTTGGTTTCTTCAAACGGAACCT | |
| | | RALSCNVSSPIFKA | GAAGGAGAAGATGGAGGCTGGCAGAGGTGTCCCGAATGGAATCCCTGCAGAA | |
| | | GHSVALQMMFNT | GACTCTGAGCAGCTGGCATCTGGGCAAGAGGCTGGGGATCCCGGCTGCCTGA | |
| | | LVNSSWGDSVEL | AGCCCCTCCATGAGAAGGACTCTGAGAGTGGTGGTGGCAAGGACTGAGTCCA | |
| | | HANVTCNNEDSD | GGCCTGTGAGGTGCAGAGTGCCCAGAACTGGACTCAGGATGCCCAGGGCCAC | |
| | | LLEDNSATTIIPILY | TCTGCCTCTGCCTGCATTCTGCCGTGTGCCCTCGGGCGAGTCACTGCCTCTCCC | |
| | | PINILIQDQEDSTL | TGGCCCTCAGTTTCCCTATCTCGAACATGGAACTCATTCCTGCCTGTCTCCTTT | |
| | | YVSFTPKGPKIHQ | GCAGGCTCATAGGGAAGACCTGCTGAGGGACCAGCCAAGAGGGCTGCAAAAG | |
| | | VKHMYQVRIQPSI | TGAGGGCTTGTCATTACCAGACGGTTCACCAGCCTCTCTTGGTTTCCTTCCTTG | |
| | | HDHNIPTLEAVVG | GAAGAGAATGTCTGATCTAAATGTGGAGAAACTGTAGTCTCAGGACCTAGGG | |
| | | VPQPPSEGPITHQ | ATGTTCTGGCCCTCACCCCTGCCCTGGGATGTCCACAGATGCCTCCACCCCCC | |
| | | WSVQMEPPVPCH | AGAACCTGTCCTTGCACACTCCCCTGCACTGGAGTCCAGTCTCTTCTGCTGGCA | |
| | | YEDLERLPDAAEP | GAAAGCAAATGTGACCTGTGTCACTACGTGACTGTGGCACACGCCTTGTTCTT | |
| | | CLPGALFRCPVVF | GGCCAAAGACCAAATTCCTTGGCATGCCTTCCAGCACCCTGCAAAATGAGACC | |
| | | RQEILVQVIGTLEL | CTCGTGGCCTTCCCCAGCCTCTTCTAGAGCCGTGATGCCTCCCTGTTGAAGCTC | |
| | | VGELEASSMFSLCS | TGGTGACACCAGCCTTTCTCCCAGGCCAGGCTCCTTCCTGTCTTCCTGCATTCA | |
| | | SLSISFNSSKHFHL | CCCAGACAGCTCCCTCTGCCTGAACCTTCCATCTCGCCACCCCTCCTTCCTTGA | |
| | | YGSNASLAQVVM | CCAGCAGATCCCAGCTCACGTCACACTTGGTTGGGTCCTCACATCTTTCACACT | |
| | | KVDVVYEKQMLY | TCCACCAGCCTGCACTACTCCCTCAAAGCACACGTCATGTTTCTTCATCCGGCA | |
| | | LYVLSGIGGLLLL | GCCTGGATGTTTTTTCCCTGTTTAATGATTGACGTACTTAGCAGCTATCTCTCA | |
| | | LLIFIVLYKVGFFK | GTGAACTGTGAGGGTAAAGGCTATACTTGTCTTGTTCACCTTGGGATGATGCC | |
| | | RNLKEKMEAGRG | TCATGATATGTCAGGGCGTGGGACATCTAGTAGGTGCTTGACATAATTTCACT | |
| | | VPNGIPAEDSEQL | GAATTAATGACAGAGCCAGTGGGAAGATACAGAAAAAGAGGGGCTGGGCTG | |
| | | ASGQEAGDPGCL | GGCGCGGTGGTTCACGCCTGTAATCCCAGCACTTTGGGAGGCCAAGGAGGGT | |
| | | KPLHEKDSESGGG | GGATCACCTGAGGTCAGGAGTTAGAGGCCAGCCTGGCGAAACCCCATCTCTA | |
| | | KD | CTAAAAATACAAAATCCAGGCGTGGTGGCACACACCTGTAGTCCCAGCTACTC | |
| | | | AGGAGGTTGAGGTAGGAGAATTGCTTGAACCTGGGAGGTGGAGGTTGCAGTG | |
| | | | AGCCAAGATTGCGCCATTGCACTCCAGCCTGGGCAACACAGCGAGACTCCGTC | |
| | | | TCAAGGAAAAAATAAAAATAAAAAGCGGGCACGGGCCCGTGACATCCCCACC | |
| | | | CTTGGAGGCTGTCTTCTCAGGCTCTGCCCTGCCCTAGCTCCACACCCTCTCCCA | |
| | | | GGACCCATCACGCCTGTGCAGTGGCCCCCACAGAAAGACTGAGCTCAAGGTG | |
| | | | GGAACCACGTCTGCTAACTTGGAGCCCCAGTGCCAAGCACAGTGCCTGCATGT | |
| | | | ATTTATCCAATAAATGTGAAATTCTGTCCAAAAAAAAAAAAAAAAAA | |
| 39 | CXCL13 | >NP_006410.1 C-X- | SEQ ID NO: 89 | |
| | | C motif chemokine | >NM_006419.2 Homo sapiens C-X-C motif chemokine ligand 13 (CXCL13), mRNA | |
| | | 13 precursor [Homo | GAGAAGATGTTTGAAAAAACTGACTCTGCTAATGAGCCTGGACTCAGAGCTC | |
| | | sapiens] | AAGTCTGAACTCTACCTCCAGACAGAATGAAGTTCATCTCGACATCTCTGCTT | |
| | | MKFISTSLLLMLL | CTCATGCTGCTGGTCAGCAGCCTCTCTCCAGTCCAAGGTGTTCTGGAGGTCTAT | |
| | | VSSLSPVQGVLEV | TACACAAGCTTGAGGTGTAGATGTGTCCAAGAGAGCTCAGTCTTTATCCCTAG | |
| | | YYTSLRCRCVQES | ACGCTTCATTGATCGAATTCAAATCTTGCCCCGTGGGAATGGTTGTCCAAGAA | |
| | | SVFIPRRFIDRIQIL | AAGAAATCATAGTCTGGAAGAAGAACAAGTCAATTGTGTGTGTGGACCCTCA | |
| | | PRGNGCPRKEIIV | AGCTGAATGGATACAAAGAATGATGGAAGTATTGAGAAAAAGAAGTTCTTCA | |
| | | WKK | ACTCTACCAGTTCCAGTGTTTAAGAGAAAGATTCCCTGATGCTGATATTTCCA | |
| | | NKSIVCVDPQAEW | CTAAGAACACCTGCATTCTTCCCTTATCCCTGCTCTGGATTTTAGTTTTGTGCTT | |
| | | IQRMMEVLRKRSS | AGTTAAATCTTTTCCAGGAAAAAGAACTTCCCCATACAAATAAGCATGAGACT | |
| | | STLPVPVFKRKIP | ATGTAAAAATAACCTTGCAGAAGCTGATGGGGCAAACTCAAGCTTCTTCACTC | |
| | | | ACAGCACCCTATATACACTTGGAGTTTGCATTCTTATTCATCAGGGAGGAAAG | |
| | | | TTTCTTTGAAAATAGTTATTCAGTTATAAGTAATACAGGATTATTTTGATTATA | |
| | | | TACTTGTTGTTTAATGTTTAAAATTTCTTAGAAAACAATGGAATGAGAATTTA | |
| | | | AGCCTCAAATTTGAACATGTGGCTTGAATTAAGAAGAAAATTATGGCATATAT | |
| | | | TAAAAGCAGGCTTCTATGAAAGACTCAAAAAGCTGCCTGGGAGGCAGATGGA | |
| | | | ACTTGAGCCTGTCAAGAGGCAAAGGAATCCATGTAGTAGATATCCTCTGCTTA | |
| | | | AAAACTCACTACGGAGGAGAATTAAGTCCTACTTTTAAAGAATTTCTTTATAA | |
| | | | AATTTACTGTCTAAGATTAATAGCATTCGAAGATCCCCAGACTTCATAGAATA | |
| | | | CTCAGGGAAAGCATTTAAAGGGTGATGTACACATGTATCCTTTCACACATTTG | |
| | | | CCTTGACAAACTTCTTTCACTCACATCTTTTTCACTGACTTTTTTTGTGGGGGG | |
| | | | CGGGGCCGGGGGGACTCTGGTATCTAATTCTTTAATGATTCCTATAAATCTAA | |
| | | | TGACATTCAATAAAGTTGAGCAAACATTTTACTTAAAAAAAAAAAAAAAAAA | |
| 40 | STAT1 | >NP_009330.1 signal | SEQ ID NO: 90 | SEQ ID NO: |
| | | transducer and | >NM_007315.3 Homo sapiens signal transducer and activator of transcription 1 (STAT1), | 126 |
| | | activator of | transcript variant alpha, mRNA | NG_008294.1 |
| | | transcription 1- | GCTGAGCGCGGAGCCGCCCGGTGATTGGTGGGGGCGGAAGGGGGCCGGGCGC | |
| | | alpha/beta isoform | CAGCGCTGCCTTTTCTCCTGCCGGGTAGTTTCGCTTTCCTGCGCAGAGTCTGCG | |
| | | alpha [Homo | GAGGGGCTCGGCTGCACCGGGGGGATCGCGCCTGGCAGACCCCAGACCGAGC | |
| | | sapiens] | AGAGGCGACCCAGCGCGCTCGGGAGAGGCTGCACCGCCGCGCCCCCGCCTAG | |
| | | MSQWYELQQLDS | CCCTTCCGGATCCTGCGCGCAGAAAAGTTTCATTTGCTGTATGCCATCCTCGA | |
| | | KFLEQVHQLYDDS | GAGCTGTCTAGGTTAACGTTCGCACTCTGTGTATATAACCTCGACAGTCTTGG | |
| | | FPMEIRQYLAQWL | CACCTAACGTGCTGTGCGTAGCTGCTCCTTTGGTTGAATCCCCAGGCCCTTGTT | |
| | | EKQDWEHAANDV | GGGGCACAAGGTGGCAGGATGTCTCAGTGGTACGAACTTCAGCAGCTTGACT | |
| | | SFATIRFHDLLSQL | CAAAATTCCTGGAGCAGGTTCACCAGCTTTATGATGACAGTTTTCCCATGGAA | |
| | | DDQYSRFSLENNF | ATCAGACAGTACCTGGCACAGTGGTTAGAAAAGCAAGACTGGGAGCACGCTG | |
| | | LLQHNIRKSKRNL | CCAATGATGTTTCATTTGCCACCATCCGTTTTCATGACCTCCTGTCACAGCTGG | |
| | | QDNFQEDPIQMSM | ATGATCAATATAGTCGCTTTTCTTTGGAGAATAACTTCTTGCTACAGCATAACA | |
| | | IIYSCLKEERKILE | TAAGGAAAAGCAAGCGTAATCTTCAGGATAATTTTCAGGAAGACCCAATCCA | |
| | | NAQRFNQAQSGNI | GATGTCTATGATCATTTACAGCTGTCTGAAGGAAGAAAGGAAAATTCTGGAA | |
| | | QSTVMLDKQKEL | AACGCCCAGAGATTTAATCAGGCTCAGTCGGGGAATATTCAGAGCACAGTGA | |
| | | DSKVRNVKDKVM | TGTTAGACAAACAGAAAGAGCTTGACAGTAAAGTCAGAAATGTGAAGGACAA | |
| | | CIEHEIKSLEDLQD | GGTTATGTGTATAGAGCATGAAATCAAGAGCCTGGAAGATTTACAAGATGAA | |
| | | EYDFKCKTLQNRE | TATGACTTCAAATGCAAAACCTTGCAGAACAGAGAACACGAGACCAATGGTG | |
| | | HETNGVAKSDQK | TGGCAAAGAGTGATCAGAAACAAGAACAGCTGTTACTCAAGAAGATGTATTT | |
| | | QEQLLLKKMYLM | AATGCTTGACAATAAGAGAAAGGAAGTAGTTCACAAAATAATAGAGTTGCTG | |
| | | LDNKRKEVVHKII | AATGTCACTGAACTTACCCAGAATGCCCTGATTAATGATGAACTAGTGGAGTG | |
| | | ELLNVTELTQNAL | GAAGCGGAGACAGCAGAGCGCCTGTATTGGGGGGCCGCCCAATGCTTGCTTG | |
| | | INDELVEWKRRQ | GATCAGCTGCAGAACTGGTTCACTATAGTTGCGGAGAGTCTGCAGCAAGTTCG | |
| | | QSACIGGPPNACL | GCAGCAGCTTAAAAAGTTGGAGGAATTGGAACAGAAATACACCTACGAACAT | |
| | | DQLQNWFTIVAES | GACCCTATCACAAAAAACAAACAAGTGTTATGGGACCGCACCTTCAGTCTTTT | |
| | | LQQVRQQLKKLE | CCAGCAGCTCATTCAGAGCTCGTTTGTGGTGGAAAGACAGCCCTGCATGCCAA | |
| | | ELEQKYTYEHDPI | CGCACCCTCAGAGGCCGCTGGTCTTGAAGACAGGGGTCCAGTTCACTGTGAAG | |
| | | TKNKQVLWDRTF | TTGAGACTGTTGGTGAAATTGCAAGAGCTGAATTATAATTTGAAAGTCAAAGT | |
| | | SLFQQLIQSSFVVE | CTTATTTGATAAAGATGTGAATGAGAGAAATACAGTAAAAGGATTTAGGAAG | |
| | | RQPCMPTHPQRPL | TTCAACATTTTGGGCACGCACACAAAAGTGATGAACATGGAGGAGTCCACCA | |
| | | VLKTGVQFTVKLR | ATGGCAGTCTGGCGGCTGAATTTCGGCACCTGCAATTGAAAGAACAGAAAAA | |
| | | LLVKLQELNYNLK | TGCTGGCACCAGAACGAATGAGGGTCCTCTCATCGTTACTGAAGAGCTTCACT | |
| | | VKVLFDKDVNER | CCCTTAGTTTTGAAACCCAATTGTGCCAGCCTGGTTIGGTAATTGACCTCGAG | |
| | | NTVKGFRKFNILG | ACGACCTCTCTGCCCGTTGTGGTGATCTCCAACGTCAGCCAGCTCCCGAGCGG | |
| | | THTKVMNMEEST | TTGGGCCTCCATCCTTTGGTACAACATGCTGGTGGCGGAACCCAGGAATCTGT | |
| | | NGSLAAEFRHLQL | CCTTCTTCCTGACTCCACCATGTGCACGATGGGCTCAGCTTTCAGAAGTGCTG | |
| | | KEQKNAGTRTNE | AGTTGGCAGTTTTCTTCTGTCACCAAAAGAGGTCTCAATGTGGACCAGCTGAA | |
| | | GPLIVTEELHSLSF | CATGTTGGGAGAGAAGCTTCTTGGTCCTAACGCCAGCCCCGATGGTCTCATTC | |
| | | ETQLCQPGLVIDL | CGTGGACGAGGTTTTGTAAGGAAAATATAAATGATAAAAATTTTCCCTTCTGG | |
| | | ETTSLPVVVISNVS | CTTTGGATTGAAAGCATCCTAGAACTCATTAAAAAACACCTGCTCCCTCTCTG | |
| | | QLPSGWASILWYN | GAATGATGGGTGCATCATGGGCTTCATCAGCAAGGAGCGAGAGCGTGCCCTG | |
| | | MLVAEPRNLSFFL | TTGAAGGACCAGCAGCCGGGGACCTTCCTGCTGCGGTTCAGTGAGAGCTCCCG | |
| | | TPPCARWAQLSEV | GGAAGGGGCCATCACATTCACATGGGTGGAGCGGTCCCAGAACGGAGGCGAA | |
| | | LSWQFSSVTKRGL | CCTGACTTCCATGCGGTTGAACCCTACACGAAGAAAGAACTTTCTGCTGTTAC | |
| | | NVDQLNMLGEKL | TTTCCCTGACATCATTCGCAATTACAAAGTCATGGCTGCTGAGAATATTCCTG | |
| | | LGPNASPDGLIPW | AGAATCCCCTGAAGTATCTGTATCCAAATATTGACAAAGACCATGCCTTTGGA | |
| | | TRFCKENINDKNF | AAGTATTACTCCAGGCCAAAGGAAGCACCAGAGCCAATGGAACTTGATGGCC | |
| | | PFWLWIESILELIK | CTAAAGGAACTGGATATATCAAGACTGAGTTGATTTCTGTGTCTGAAGTTCAC | |
| | | KHLLPLWNDGCI | CCTTCTAGACTTCAGACCACAGACAACCTGCTCCCCATGTCTCCTGAGGAGTT | |
| | | MGFISKERERALL | TGACGAGGTGTCTCGGATAGTGGGCTCTGTAGAATTCGACAGTATGATGAACA | |
| | | KDQQPGTFLLRFS | CAGTATAGAGCATGAATTTTTTTCATCTTCTCTGGCGACAGTTTTCCTTCTCAT | |
| | | ESSREGAITFTWV | CTGTGATTCCCTCCTGCTACTCTGTTCCTTCACATCCTGTGTTTCTAGGGAAAT | |
| | | ERSQNGGEPDFHA | GAAAGAAAGGCCAGCAAATTCGCTGCAACCTGTTGATAGCAAGTGAATTTTTC | |
| | | VEPYTKKELSAVT | TCTAACTCAGAAACATCAGTTACTCTGAAGGGCATCATGCATCTTACTGAAGG | |
| | | FPDIIRNYKVMAA | TAAAATTGAAAGGCATTCTCTGAAGAGTGGGTTTCACAAGTGAAAAACATCC | |
| | | ENIPENPLKYLYP | AGATACACCCAAAGTATCAGGACGAGAATGAGGGTCCTTTGGGAAAGGAGAA | |
| | | NIDKDHAFGKYYS | GTTAAGCAACATCTAGCAAATGTTATGCATAAAGTCAGTGCCCAACTGTTATA | |
| | | RPKEAPEPMELDG | GGTTGTTGGATAAATCAGTGGTTATTTAGGGAACTGCTTGACGTAGGAACGGT | |
| | | PKGTGYIKTELISV | AAATTTCTGTGGGAGAATTCTTACATGTTTTCTTTGCTTTAAGTGTAACTGGCA | |
| | | SEVHPSRLQTTDN | GTTTTCCATTGGTTTACCTGTGAAATAGTTCAAAGCCAAGTTTATATACAATTA | |
| | | LLPMSPEEFDEVS | TATCAGTCCTCTTTCAAAGGTAGCCATCATGGATCTGGTAGGGGGAAAATGTG | |
| | | RIVGSVEFDSMMN | TATTTTATTACATCTTTCACATTGGCTATTTAAAGACAAAGACAAATTCTGTTT | |
| | | TV | CTTGAGAAGAGAATATTAGCTTTACTGTTTGTTATGGCTTAATGACACTAGCT | |
| | | | AATATCAATAGAAGGATGTACATTTCCAAATTCACAAGTTGTGTTTGATATCC | |
| | | | AAAGCTGAATACATTCTGCTTTCATCTTGGTCACATACAATTATTTTTACAGTT | |
| | | | CTCCCAAGGGAGTTAGGCTATTCACAACCACTCATTCAAAAGTTGAAATTAAC | |
| | | | CATAGATGTAGATAAACTCAGAAATTTAATTCATGTTTCTTAAATGGGCTACT | |
| | | | TTGTCCTTTTTGTTATTAGGGTGGTATTTAGTCTATTAGCCACAAAATTGGGAA | |
| | | | AGGAGTAGAAAAAGCAGTAACTGACAACTTGAATAATACACCAGAGATAATA | |
| | | | TGAGAATCAGATCATTTCAAAACTCATTTCCTATGTAACTGCATTGAGAACTG | |
| | | | CATATGTTTCGCTGATATATGTGTTTTTCACATTTGCGAATGGTTCCATTCTCTC | |
| | | | TCCTGTACTTTTTCCAGACACTTTTTTGAGTGGATGATGTTTCGTGAAGTATAC | |
| | | | TGTATTTTTACCTTTTTCCTTCCTTATCACTGACACAAAAAGTAGATTAAGAGA | |
| | | | TGGGTTTGACAAGGTTCTTCCCTTTTACATACTGCTGTCTATGTGGCTGTATCT | |
| | | | TGTTTTTCCACTACTGCTACCACAACTATATTATCATGCAAATGCTGTATTCTT | |
| | | | CTTTGGTGGAGATAAAGATTTCTTGAGTTTTGTTTTAAAATTAAAGCTAAAGT | |
| | | | ATCTGTATTGCATTAAATATAATATGCACACAGTGCTTTCCGTGGCACTGCAT | |
| | | | ACAATCTGAGGCCTCCTCTCTCAGTTTTTATATAGATGGCGAGAACCTAAGTTT | |
| | | | CAGTTGATTTTACAATTGAAATGACTAAAAAACAAAGAAGACAACATTAAAA | |
| | | | CAATATTGTTTCTAATTGCTGAGGTTTAGCTGTCAGTTCTTTTTGCCCTTTGGG | |
| | | | AATTCGGCATGGTTTCATTTTACTGCACTAGCCAAGAGACTTTACTTTTAAGAA | |
| | | | GTATTAAAATTCTAAAATTCAAAAAAAAAAAAAAAAAA | |
| 41 | CXCL10 | >NP_001556.2 C-X- | SEQ ID NO: 91 | |
| | | C motif chemokine | >NM_001565.3 Homo sapiens C-X-C motif chemokine ligand 10 (CXCL10), mRNA | |
| | | 10 precursor [Homo | CTTTGCAGATAAATATGGCACACTAGCCCCACGTTTTCTGAGACATTCCTCAA | |
| | | sapiens] | TTGCTTAGACATATTCTGAGCCTACAGCAGAGGAACCTCCAGTCTCAGCACCA | |
| | | MNQTAILICCLIFL | TGAATCAAACTGCCATTCTGATTTGCTGCCTTATCTTTCTGACTCTAAGTGGCA | |
| | | TLSGIQGVPLSRTV | TTCAAGGAGTACCTCTCTCTAGAACTGTACGCTGTACCTGCATCAGCATTAGT | |
| | | RCTCISISNQPVNP | AATCAACCTGTTAATCCAAGGTCTTTAGAAAAACTTGAAATTATTCCTGCAAG | |
| | | RSLEKLEIIPASQF | CCAATTTTGTCCACGTGTTGAGATCATTGCTACAATGAAAAAGAAGGGTGAGA | |
| | | CPRVEIIATMKKK | AGAGATGTCTGAATCCAGAATCGAAGGCCATCAAGAATTTACTGAAAGCAGT | |
| | | GEKRCLNPESKAI | TAGCAAGGAAAGGTCTAAAAGATCTCCTTAAAACCAGAGGGGAGCAAAATCG | |
| | | KNLLKAVSKERSK | ATGCAGTGCTTCCAAGGATGGACCACACAGAGGCTGCCTCTCCCATCACTTCC | |
| | | RSP | CTACATGGAGTATATGTCAAGCCATAATTGTTCTTAGTTTGCAGTTACACTAA | |
| | | | AAGGTGACCAATGATGGTCACCAAATCAGCTGCTACTACTCCTGTAGGAAGGT | |
| | | | TAATGTTCATCATCCTAAGCTATTCAGTAATAACTCTACCCTGGCACTATAATG | |
| | | | TAAGCTCTACTGAGGTGCTATGTTCTTAGTGGATGTTCTGACCCTGCTTCAAAT | |
| | | | ATTTCCCTCACCTTTCCCATCTTCCAAGGGTACTAAGGAATCTTTCTGCTTTGG | |
| | | | GGTTTATCAGAATTCTCAGAATCTCAAATAACTAAAAGGTATGCAATCAAATC | |
| | | | TGCTTTTTAAAGAATGCTCTTTACTTCATGGACTTCCACTGCCATCCTCCCAAG | |
| | | | GGGCCCAAATTCTTTCAGTGGCTACCTACATACAATTCCAAACACATACAGGA | |
| | | | AGGTAGAAATATCTGAAAATGTATGTGTAAGTATTCTTATTTAATGAAAGACT | |
| | | | GTACAAAGTAGAAGTCTTAGATGTATATATTTCCTATATTGTTTTCAGTGTACA | |
| | | | TGGAATAACATGTAATTAAGTACTATGTATCAATGAGTAACAGGAAAATTTTA | |
| | | | AAAATACAGATAGATATATGCTCTGCATGTTACATAAGATAAATGTGCTGAAT | |
| | | | GGTTTTCAAAATAAAAATGAGGTACTCTCCTGGAAATATTAAGAAAGACTATC | |
| | | | TAAATGTTGAAAGATCAAAAGGTTAATAAAGTAATTATAACTAAGAAAAAAA | |
| | | | AAAAA | |
| 42 | CXCL11 | >NP_001289052.1 | SEQ ID NO: 92 | |
| | | C-X-C motif | >NM_005409.4 Homo sapiens C-X-C motif chemokine ligand 11 (CXCL11), transcript | |
| | | chemokine 11 | variant 1, mRNA | |
| | | isoform 2 precursor | AGAGAACAAAACAGAAACTCTTGGAAGCAGGAAAGGTGCATGACTCAAAGA | |
| | | [Homo sapiens] | GGGAAATTCCTGTGCCATAAAAGGATTGCTGGTGTATAAAATGCTCTATATAT | |
| | | MSVKGMAIALAVI | GCCAATTATCAATTTCCTTTCATGTTCAGCATTTCTACTCCTTCCAAGAAGAGC | |
| | | LCATVVQGFPMF | AGCAAAGCTGAAGTAGCAGCAGCAGCACCAGCAGCAACAGCAAAAAACAAA | |
| | | KRGRCLCIGPGVK | CATGAGTGTGAAGGGCATGGCTATAGCCTTGGCTGTGATATTGTGTGCTACAG | |
| | | AVKVADIEKASIM | TTGTTCAAGGCTTCCCCATGTTCAAAAGAGGACGCTGTCTTTGCATAGGCCCT | |
| | | YPSNNCDKIEVIIT | GGGGTAAAAGCAGTGAAAGTGGCAGATATTGAGAAAGCCTCCATAATGTACC | |
| | | LKENKGQRCLNP | CAAGTAACAACTGTGACAAAATAGAAGTGATTATTACCCTGAAAGAAAATAA | |
| | | KSKQARLIIKLKER | AGGACAACGATGCCTAAATCCCAAATCGAAGCAAGCAAGGCTTATAATCAAA | |
| | | IFKNIKTYEVLEKS | AAAGTTGAAAGAAAGAATTTTTAAAAATATCAAAACATATGAAGTCCTGGAA | |
| | | I | AAGAGCATCTGAAAAACCTAGAACAAGTTTAACTGTGACTACTGAAATGACA | |
| | | | AGAATTCTACAGTAGGAAACTGAGACTTTTCTATGGTTTTGTGACTTTCAACTT | |
| | | | TTGTACAGTTATGTGAAGGATGAAAGGTGGGTGAAAGGACCAAAAACAGAAA | |
| | | | TACAGTCTTCCTGAATGAATGACAATCAGAATTCCACTGCCCAAAGGAGTCCA | |
| | | | ACAATTAAATGGATTTCTAGGAAAAGCTACCTTAAGAAAGGCTGGTTACCATC | |
| | | | GGAGTTTACAAAGTGCTTTCACGTTCTTACTTGTTGCATTATACATTCATGCAT | |
| | | | TTCTAGGCTAGAGAACCTTCTAGATTTGATGCTTACAACTATTCTGTTGTGACT | |
| | | | ATGAGAACATTTCTGTCTCTAGAAGTCATCTGTCTGTATTGATCTTTATGCTAT | |
| | | | ATTACTATCTGTGGTTACGGTGGAGACATTGACATTATTACTGGAGTCAAGCC | |
| | | | CTTATAAGTCAAAAGCATCTATGTGTCGTAAAACATTCCTCAAACATTTTTTCA | |
| | | | TGCAAATACACACTTCTTTCCCCAAACATCATGTAGCACATCAATATGTAGGG | |
| | | | AGACATTCTTATGCATCATTTGGTTYGTTTTATAACCAATTCATTAAATGTAAT | |
| | | | TCATAAAATGTACTATGAAAAAAATTATACGCTATGGGATACTGGCAAAAGT | |
| | | | GCACATATTTCATAACCAAATTAGTAGCACCAGTCTTAATTTGATGITTTTCAA | |
| | | | CTTTTATTCATTGAGATGTTTTGAAGCAATTAGGATATGTGTGTTTACTGTACT | |
| | | | TTTTGTTTTGATCCGTTTGTATAAATGATAGCAATATCTTGGACACATCTGAAA | |
| | | | TACAAAATGTTTTTGTCTACCAAAGAAAAATGTTGAAAAATAAGCAAATGTAT | |
| | | | ACCTAGCAATCACTTTTACTTTTTGTAATTCTGTCTCTTAGAAAAATACATAAT | |
| | | | CTAATCAATTTCTTTGTTCATGCCTATATACTGTAAAATTTAGGTATACTCAAG | |
| | | | ACTAGTTTAAAGAATCAAAGTCATTTTTTTCTCTAATAAACTACCACAACCTTT | |
| | | | CTTTTTTAAAAAAAAAAA | |
| 43 | LAG3 | >NP_002277.4 | SEQ ID NO: 93 | |
| | | lymphocyte | >NM_002286.5 Homo sapiens lymphocyte activating 3 (LAG3), mRNA | |
| | | activation gene 3 | ACAGGGGTGAAGGCCCAGAGACCAGCAGAACGGCATCCCAGCCACGACGGCC | |
| | | protein precursor | ACTTTGCTCTGTCTGCTCTCCGCCACGGCCCTGCTCTGTTCCCTGGGACACCCC | |
| | | [Homo sapiens] | CGCCCCCACCTCCTCAGGCTGCCTGATCTGCCCAGCTTTCCAGCTTTCCTCTGG | |
| | | MWEAQFLGLLFL | ATTCCGGCCTCTGGTCATCCCTCCCCACCCTCTCTCCAAGGCCCTCTCCTGGTC | |
| | | QPLWVAPVKPLQP | TCCCTTCTTCTAGAACCCCTTCCTCCACCTCCCTCTCTGCAGAACTTCTCCTTTA | |
| | | GAEVPVVWAQEG | CCCCCCACCCCCCACCACTGCCCCCTTTCCTTTTCTGACCTCCTTTTGGAGGGC | |
| | | APAQLPCSPTWLQ | TCAGCGCTGCCCAGACCATAGGAGAGATGTGGGAGGCTCAGTTCCTGGGCTTG | |
| | | DLSLLRRAGVTW | CTGTTTCTGCAGCCGCTTTGGGTGGCTCCAGTGAAGCCTCTCCAGCCAGGGGC | |
| | | QHQPDSGPPAAAP | TGAGGTCCCGGTGGTGTGGGCCCAGGAGGGGGCTCCTGCCCAGCTCCCCTGCA | |
| | | GHPLAPGPHPAAP | GCCCCACAATCCCCCTCCAGGATCTCAGCCTTCTGCGAAGAGCAGGGGTCACT | |
| | | SSWGPRPRRYTVL | TGGCAGCATCAGCCAGACAGTGGCCCGCCCGCTGCCGCCCCCGGCCATCCCCT | |
| | | SVGPGGLRSGRLP | GGCCCCCGGCCCTCACCCGGCGGCGCCCTCCTCCTGGGGGCCCAGGCCCCGCC | |
| | | LQPRVQLDERGRQ | GCTACACGGTGCTGAGCGTGGGTCCCGGAGGCCTGCGCAGCGGGAGGCTGCC | |
| | | RGDFSLWLRPARR | CCTGCAGCCCCGCGTCCAGCTGGATGAGCGCGGCCGGCAGCGCGGGGACTTC | |
| | | ADAGEYRAAVHL | TCGCTATGGCTGCGCCCAGCCCGGCGCGCGGACGCCGGCGAGTACCGCGCCG | |
| | | RDRALSCRLRLRL | CGGTGCACCTCAGGGACCGCGCCCTCTCCTGCCGCCTCCGTCTGCGCCTGGGC | |
| | | GQASMTASPPGSL | CAGGCCTCGATGACTGCCAGCCCCCCAGGATCTCTCAGAGCCTCCGACTGGGT | |
| | | RASDWVILNCSFS | CATTTTGAACTGCTCCTTCAGCCGCCCTGACCGCCCAGCCTCTGTGCATTGGTT | |
| | | RPDRPASVHWTR | CCGGAACCGGGGCCAGGGCCGAGTCCCTGTCCGGGAGTCCCCCCATCACCAC | |
| | | NRGQGRVPVRESP | TTAGCGGAAAGCTTCCTCTTCCTGCCCCAAGTCAGCCCCATGGACTCTGGGCC | |
| | | HHHLAESFLFLPQ | CTGGGGCTGCATCCTCACCTACAGAGATGGCTTCAACGTCTCCATCATGTATA | |
| | | VSPMDSGPWGCIL | ACCTCACTGTTCTGGGTCTGGAGCCCCCAACTCCCTTGACAGTGTACGCTGGA | |
| | | TYRDGFNVSIMYN | GCAGGTTCCAGGGTGGGGCTGCCCTGCCGCCTGCCTGCTGGTGTGGGGACCCG | |
| | | LTVLGLEPPTPLTV | GTCTTTCCTCACTGCCAAGTGGACTCCTCCTGGGGGAGGCCCTGACCTCCTGG | |
| | | YAGAGSRVGLPC | TGACTGGAGACAATGGCGACTTTACCCTTCGACTAGAGGATGTGAGCCAGGCC | |
| | | RLPAGVGTRSFLT | CAGGCTGGGACCTACACCTGCCATATCCATCTGCAGGAACAGCAGCTCAATGC | |
| | | AKNYIPPGGGPDL | CACTGTCACATTGGCAATCATCACAGTGACTCCCAAATCCTTTGGGTCACCTG | |
| | | LVTGDNGDFTLRL | GATCCCTGGGGAAGCTGCTTTGTGAGGTGACTCCAGTATCTGGACAAGAACGC | |
| | | EDVSQAQAGTYT | TTTGTGTGGAGCTCTCTGGACACCCCATCCCAGAGGAGTTTCTCAGGACCTTG | |
| | | CHEILDEQQLNAT | GCTGGAGGCACAGGAGGCCCAGCTCCTTTCCCAGCCTTGGCAATGCCAGCTGT | |
| | | VTLAIITVTPKSFG | ACCAGGGGGAGAGGCTTCTTGGAGCAGCAGTGTACTTCACAGAGCTGTCTAG | |
| | | SPGSLGKLLCEVT | CCCAGGTGCCCAACGCTCTGGGAGAGCCCCAGGTGCCCTCCCAGCAGGCCAC | |
| | | PVSGQERFVWSSL | CTCCTGCTGTTTCTCATCCTTGGTGTCCTTTCTCTGCTCCTTTTGGTGACTGGAG | |
| | | DTPSQRSFSGPWL | CCTTTGGCTTTCACCTTTGGAGAAGACAGTGGCGACCAAGACGATTTTCTGCC | |
| | | EADEAQLLSQPW | TTAGAGCAAGGGATTCACCCTCCGCAGGCTCAGAGCAAGATAGAGGAGCTGG | |
| | | QCQLYQGERLLG | AGCAAGAACCGGAGCCGGAGCCGGAGCCGGAACCGGAGCCCGAGCCCGAGC | |
| | | AAVYFTELSSPGA | CCGAGCCGGAGCAGCTCTGACCTGGAGCTGAGGCAGCCAGCAGATCTCAGCA | |
| | | QRSGRAPGALPAG | GCCCAGTCCAAATAAACTCCCTGTCAGCAGCAAAAA | |
| | | HLLLFLILGVLSLL | | |
| | | LLVTGAFGFHLW | | |
| | | RRQWTRYRRESALE | | |
| | | QGIHPPQAQSMEE | | |
| | | LEQEPEPEPEPEPE | | |
| | | PEPEPEPEQL | | |
| 44 | IRF1 | >NP_001341854.1 | SEQ ID NO: 94 | SEQ ID NO: |
| | | interferon regulatory | >NM_001354925.1 Homo sapiens interferon regulatory factor 1 (IRF1), transcript | 127 |
| | | factor 1 isoform 3 | variant 3, mRNA | NG_011450.1 |
| | | [Homo sapiens] | AGAGCTCGCCACTCCTTAGTCGAGGCAAGACGTGCGCCCGAGCCCCGCCGAA | |
| | | MPITRMRMRPWL | CCGAGGCCACCCGGAGCCGTGCCCAGTCCACGCCGGCCGTGCCCGGCGGCCT | |
| | | EMQINSNQIPGLI | TAAGAACCCGGCAACCTCTGCCTTCTTCCCTCTTCCACTCGGAGTCGCGCTCCG | |
| | | WINKEEMIFQIPW | CGCGCCCTCACTGCAGCCCCTGCGTCGCCGGGACCCTCGCGCGCGACCGCCGA | |
| | | KHAAKHGWDINK | ATCGCTCCTGCAGCAGAGCCAACATGCCCATCACTCGGATGCGCATGAGACCC | |
| | | DACLFRSWAIHTG | TGGCTAGAGATGCAGATTAATTCCAACCAAATCCCGGGGCTCATCTGGATTAA | |
| | | RYKAGEKEPDPKT | TAAAGAGGAGATGATCTTCCAGATCCCATGGAAGCATGCTGCCAAGCATGGC | |
| | | WKANFRCAMNSL | TGGGACATCAACAAGGATGCCTGTTTGTTCCGGAGCTGGGCCATTCACACAGG | |
| | | PDIEEVKDQSRNK | CCGATACAAAGCAGGGGAAAAGGAGCCAGATCCCAAGACGTGGAAGGCCAA | |
| | | GSSAVRVYRMLPP | CTTTCGCTGTGCCATGAACTCCCTGCCAGATATCGAGGAGGTGAAAGACCAGA | |
| | | LTKNQRKERKSKS | GCAGGAACAAGGGCAGCTCAGCTGTGCGAGTGTACCGGATGCTTCCACCTCTC | |
| | | SRDAKSKAKRKSC | ACCAAGAACCAGAGAAAAGAAAGAAAGTCGAAGTCCAGCCGAGATGCTAAG | |
| | | GDSSPDTFSDGLSS | AGCAAGGCCAAGAGGAAGTCATGTGGGGATTCCAGCCCTGATACCTTCTCTGA | |
| | | STLPDDHSSYTVP | TGGACTCAGCAGCTCCACTCTGCCTGATGACCACAGCAGCTACACAGTTCCAG | |
| | | GYMQDLEVEQAL | GCTACATGCAGGACTTGGAGGTGGAGCAGGCCCTGACTCCAGCACTGTCGCC | |
| | | TPALSPCAVSSTLP | ATGTGCTGTCAGCAGCACTCTCCCCGACTGGCACATCCCAGTGGAAGTTGTGC | |
| | | DWHIPVEVVPDST | CGGACAGCACCAGTGATCTGTACAACTTCCAGGTGTCACCCATGCCCTCCACC | |
| | | SDLYNFQVSPMPS | TCTGAAGGGGATATTGGGCTGAGTCTACAGCGTGTCTTCACAGATCTGAAGAA | |
| | | TSEGDIGLSLQRVF | CATGGATGCCACCTGGCTGGACAGCCTGCTGACCCCAGTCCGGTTGCCCTCCA | |
| | | TDLKNMDATWLD | TCCAGGCCATTCCCTGTGCACCGTAGCAGGGCCCCTGGGCCCCTCTTATTCCTC | |
| | | SLLTPVRLPSIQAIP | TAGGCAAGCAGGACCTGGCATCATGGTGGATATGGTGCAGAGAAGCTGGACT | |
| | | CAP | TCTGTGGGCCCCTCAACAGCCAAGTGTGACCCCACTGCCAAGTGGGGATGGG | |
| | | | GCCTCCCTCCTTGGGTCATTGACCTCTCAGGGCCTGGCAGGCCAGTGTCTGGG | |
| | | | TTTTTCTTGTGGTGTAAAGCTGGCCCTGCCTCCTGGGAAGATGAGGTTCTGAG | |
| | | | ACCAGTGTATCAGGTCAGGGACTTGGACAGGAGTCAGTGTCTGGCTTTTTCCT | |
| | | | CTGAGCCCAGCTGCCTGGAGAGGGTCTCGCTGTCACTGGCTGGCTCCTAGGGG | |
| | | | AACAGACCAGTGACCCCAGAAAAGCATAACACCAATCCCAGGGCTGGCTCTG | |
| | | | CACTAAGAGAAAATTGCACTAAATGAATCTCGTTCCCAAAGAACTACCCCCTT | |
| | | | TTCAGCTGAGCCCTGGGGACTGTTCCAAAGCCAGTGAAATGTGAAGGAAAGT | |
| | | | GGGGTCCTTCGGGGCGATGCTCCCTCAGCCTCAGAGGAGCTCTACCCTGCTCC | |
| | | | CTGCTTTGGCTGAGGGGCTTGGGAAAAAAACTTGGCACTTTTTCGTGTGGATC | |
| | | | TTGCCACATTTCTGATCAGAGGTGTACACTAACATTTCCCCCGAGCTCTTGGCC | |
| | | | TTTGCATTTATTTATACAGTGCCTTGCTCGGCGCCCACCACCCCCTCAAGCCCC | |
| | | | AGCAGCCCTCAACAGGCCCAGGGAGGGAAGTGTGAGCGCCTTGGTATGACTT | |
| | | | AAAATTGGAAATGTCATCTAACCATTAAGTCATGTGTGAACACATAAGGACGT | |
| | | | GTGTAAATATGTACATTTGTCTTTTTATAAAAAGTAAATTGTTTATAAGGGGTG | |
| | | | TGGCCTTTTTAGAGAGAAATTTAACTTGTAGATGATTTTACTTTTTATGGAAAC | |
| | | | ACTGATGGACTTATTATTGGCATCCCGCCTGAACTTGACTTTGGGGTGAACAG | |
| | | | GGACATGCATCTATTATAAAATCCTTTCGGCCAGGCGCGGTGGCTCACACCTG | |
| | | | TAATCCCAGCACTTTGGGAGGCCGAGATGGGTGGATCACCTGAGGTCAGGAG | |
| | | | TTCGAGACCAGCCTGGTGAAACTCCATTTCTACTAAAAATGCAAAAATTAGCT | |
| | | | GGGCGTGGTTGCGGGTGCTTGTAATCCCAGCTACTCAGGAGGCTGAGGCAAG | |
| | | | AGAATCGCTTGAACCTGGGAGGTGGAGGTTGCAGTGAGCCGAGAACATGCCA | |
| | | | TTGCACTCCAGCCCGGGCACCAAAAAAAAAAAAAAAAAAAAAAACCTTTCAT | |
| | | | TTGGCCGGGCATGGTGGCTTATGCCTGTAATCCTGGCACTTTGGGAGGCCAAG | |
| | | | GTGGGCAGATCACCTGAGGTCAGGAGTTTGAGACCAGCCTGGCCAACATGGT | |
| | | | GAAACCTCATCTCTACTAAAAATACAAAAATTAGGCCGGGCACGGTGGCTCA | |
| | | | CGCCTGTAATCCCAGCACTTTGGGAGGCAGAGGCGGGCGGATCACGAGGTCA | |
| | | | GGAGATCAAGACCATCCTGGCTAACACGGTGAAACCCCGTCTCTACTAAAAAT | |
| | | | ATAAAAAATTAGCCGGGCCTAGTGGCGGGTGCCTGTAGTCCCAGCTACTCGGG | |
| | | | AGGCTGAGGCAGGAGAATGGCATGAACCCCGGAGGCAGAGCTTGCAGTGAGC | |
| | | | CGAGATTGCACCACTGCACTACAGCCTGGGCGACAGAGCGAGACTCCGTCTC | |
| | | | AAAAAAAAAAAAAAAAATTAGCCGGGCCTGGTGGCGGGCGCCTGTAATCCCA | |
| | | | GCTACTGTGGAGGCTGAAGCACAAGAATCACTTGAACCCGGGAGATGGAGGT | |
| | | | TGCAGTGAGCTGAGACTGTGCCACTGCACTCCAGCCTGGGTGACAAGAGTGA | |
| | | | GACTTTGTCTCAAAAAAAAAAAAATCCTTTTGTTTATGTTCACATAGACAATG | |
| | | | GCAGAAGGAGGGGACATTCCTGTCATAGGAACATGCTTATATAAACATAGTC | |
| | | | ACCTGTCCTTGACTATCACCAGGGCTGTCAGTTGATTCTGGGCTCCTGGGGCC | |
| | | | CAAGGAGTGTTAAGTTTTGAGGCATGTGCCATAGGTGATGTGTCCTGCTAACA | |
| | | | CACAGATGCTGCTCCAAAAAGTCAGTTGATATGACACAGTCACAGACAGAAC | |
| | | | AGTCAGCAGCCCAAGAAAGGTCCTCACGGCTGCTGTGCTGGGTAGCACTTGCC | |
| | | | ATCCAGTTTCTAGAGTGATGAAATGCTCTGTCTGTACCGTTCAATACAGTAGG | |
| | | | CACTGGCACTAGCCACATGTGCCAGCTAAGCACTTGAAATGTGGCCAGTGCAA | |
| | | | TAAGGAATTGAACTTTTAATTGCATTTAATAAACTGTATGTAAATAGTCA | |
| 45 | TAGAP | >NP_687034.1 T-cell | SEQ ID NO: 95 | SEQ ID NO: |
| | | activation Rho | >NM_152133.2 Homo sapiens Tcell activation RhoGTPase activating protein (TAGAP), | 128 |
| | | GTPase-activating | transcript variart 1, mRNA | NG_011524A |
| | | protein isoform a | GACATAGCTGCCCTAAAAGGAATGAGGAAGCGAGAGCTCTCCAGTGTCTGGC | |
| | | [Homo sapiens] | TGGCTCCGTCCGTGTGACAGCCCATGATGTTCTTTCCGGTCTCTGTAATATTCT | |
| | | MGALEMQDEEDR | GAATTTCCACCTGCCCGCCCCTTCGCTTATAATGCAGAGCATGTGAAGGGAGA | |
| | | IEALKQVADKLPR | CCGGCTCGGTCTCTCTCTCTCCCAGTGGACTAGAAGGAGCAGAGAGTTATGCT | |
| | | PNLLLLKHLVYVL | GTTTCTCCCATTCTTTACAGCTCACCGGATGTAAAAGAACTCTGGCTAGAGAC | |
| | | HLISKNSEVNRMD | CCTCCAAGGACAGAGGCACAGCCACACGGGAGTGAAATCCACCCCTGGACAG | |
| | | SSNLAICIGPNMLT | TCAGCCGCAATACTGATGAAGCTGAGAAGCAGCCACAATGCTTCAAAAACAC | |
| | | LENDQSLSFEAQK | TAAACGCCAATAATATGGAGACACTAATCGAATGTCAATCAGAGGGTGATAT | |
| | | DLNNKVKTLVEFL | CAAGGAACATCCCCTGTTGGCATCATGTGAGAGTGAAGACAGTATTTGCCAGC | |
| | | IDNCFEIFGENIPV | TCATTGGACATTCTCACTATTCTATGCCTTAAAGGCCCTTCAACGGAAGGGAT | |
| | | HSMTSDDSLEHTD | ATTCAGGAGAGCAGCCAACGAGAAAGCCCGTAAGGAGCTGAAGGAGGAGCT | |
| | | SSDVSTLQNDSAY | CAACTCTGGGGATGCGGTGGATCTGGAGAGGCTCCCCGTGCACCTCCTCGCTG | |
| | | DSNDPDVESNSSS | TGGTCTTTAAGGACTTCCTCAGAAGTATCCCCCGGAAGCTACTTTCAAGCGAC | |
| | | GISSPSRQPQVPM | CTCTTTGAGGAGTGGATGGGTGCTCTGGAGATGCAGGACGAGGAGGACAGAA | |
| | | ATAAGLDSAGPQ | TCGAGGCCCTGAAACAGGTTGCAGATAAGCTCCCCCGGCCCAACCTCCTGCTA | |
| | | DAREVSPEPIVSTV | CTCAAGCACTTGGTCTATGTGCTGCACCTCATCAGCAAGAACTCTGAGGTGAA | |
| | | ARLKSSLAQPDRR | CAGGATGGACTCCAGCAATCTGGCCATCTGCATTGGACCCAACATGCTCACCC | |
| | | YSEPSMPSSQECL | TGGAGAATGACCAGAGCCTGTCATTTGAAGCCCAGAAGGACCTGAACAACAA | |
| | | ESRVTNQTLTKSE | GGTGAAGACACTGGTGGAATTCCTCATTGATAACTGCTTTGAAATATTTGGGG | |
| | | GDFPVPRVGSRLE | AGAACATTCCAGTGCATTCCAGTATCACTTCTGATGACTCCCTGGAGCACACT | |
| | | SEEAEDPFPEEVFP | GACAGTTCAGATGTGTCGACCCTGCAGAATGACTCAGCCTACGACAGCAACG | |
| | | AVQGKTKRPVDL | ACCCTGATGTGGAATCCAACAGCAGCAGTGGCATCAGCTCTCCCAGCAGGCA | |
| | | KIKNLAPGSVLPR | GCCCCAGGTGCCCATGGCCACAGCTGCTGGCTTGGATAGCGCGGGCCCACAG | |
| | | ALVLKAFSSSSLD | GATGCCCGAGAGGTCAGCCCAGAGCCCATTGTGAGCACCGTGGCCAGGCTGA | |
| | | ASSDSSPVASPSSP | AAAGCTCCCTCGCACAGCCCGATAGGAGATACTCAGAGCCCAGCATGCCATC | |
| | | KRNFFSRHQSFTT | CTCCCAGGAGTGCCTCGAGAGCCGGGTGACAAACCAAACACTAACAAAGAGT | |
| | | KTEKGKPSREIKK | GAAGGGGACTTCCCCGTGCCCCGGGTAGGCTCTCGTTTGGAAAGTGAGGAGG | |
| | | HSMSFTFAPHKKV | CTGAAGACCCATTTCCAGAGGAGGTCTTCCCTGCAGTGCAAGGCAAAACCAA | |
| | | LTKNLSAGSGKSQ | GAGGCCGGTGGACCTGAAGATCAAGAACTTGGCCCCGGGTTCGGTGCTCCCG | |
| | | DFTRDHVPRGVR | CGGGCACTGGTTCTCAAAGCCTTCTCCAGCAGCTCGCTGGACGCGTCCTCTGA | |
| | | KESQLAGRIVQEN | CAGCTCGCCCGTGGCTTCTCCTTCCAGTCCCAAAAGAAATTTCTTCAGCAGAC | |
| | | GCETHNQTARGFC | ATCAGTCTTTCACCACAAAGACAGAGAAAGGCAAGCCCAGCCGAGAAATTAA | |
| | | LRPHALSVDDVFQ | AAAGCACTCCATGTCTTTCACCTTTGCCCCTCACAAAAAAGTGCTGACCAAAA | |
| | | GADWERPGSPPSY | ACCTCAGCGCGGGCTCTGGGAAATCGCAAGACTTTACCAGGGACCACGTCCC | |
| | | EEAMQGPAARLV | GAGGGGTGTCAGAAAGGAAAGCCAGCTTGCCGGCCGAATCGTGCAGGAAAAT | |
| | | ASESQTVGSMTVG | GGGTGTGAAACCCACAACCAAACAGCCCGCGGCTTCTGCCTGAGACCCCACG | |
| | | WIRARMLEAHCL | CCCTCTCGGTGGATGATGTGTTCCAGGGAGCTGACTGGGAGAGGCCTGGAAG | |
| | | LPPLPPAHHVEDS | CCCACCCTCTTATGAAGAGGCCATGCAGGGCCCGGCAGCCAGACTAGTGGCCT | |
| | | RHRGSKEPLPGHG | CCGAGAGCCAGACCGTGGGGAGCATGACGGTGGGGAGCATGAGGGCGAGGA | |
| | | LSPLPERWKQSRT | TGCTGGAGGCGCACTGCCTCCTACCCCCTCTTCCACCTGCTCACCACGTAGAG | |
| | | VHASGDSLGHVS | GACTCAAGACACAGGGGCAGCAAAGAGCCACTCCCTGGCCACGGACTCTCTC | |
| | | GPGRPELLPLRTV | CCCTGCCTGAGCGATGGAAACAGAGCAGAACTGTCCATGCTTCTGGGGACTCT | |
| | | SESVQRNKRDCLV | CTGGGGCACGTGTCTGGCCCAGGGAGACCTGAGCTCCTCCCGCTGAGGACCGT | |
| | | RRCSQPVFEADQF | CTCCGAGTCCGTGCAGAGGAATAAGCGGGACTGTCTCGTGCGACGATGTAGC | |
| | | QYAKESYI | CAGCCGGTCTTTGAGGCTGACCAATTCCAATATGCCAAAGAATCGTATATTTA | |
| | | | GGAGGGAGGCCATACGCCATGCCATAGCTTGTGCTATCTGTAAATATGAGACT | |
| | | | TGTAAAGAACTGCCTGTAGATTGTTYTTAAAAGGTCTTGAATAAGCTCCTTGA | |
| | | | GAAAGTTGTGGAAAGCCCTCCTCAGTGAGGATAGCTACACCATGGCCATGGC | |
| | | | GCATCAGATAGTCTCTGTGTACCTGGATTTGTGCAATATGTAAAAATGTATCA | |
| | | | AATGTATTATAGATAAGGTGTTAGGTGCAAAGGATGTCTAATAATCCCTGCAC | |
| | | | ACGTTTTGAACTTGCAGTGAAGTACACTGCTGTTCCTTGCTTCCTGGGGCACTT | |
| | | | TTCTCTTGGTTAGTGTTTAAAAATTATCTTCGCTTTTTTAATGTGGCCTCAAAT | |
| | | | GTCATGCCAATTTTCACATCTTCCACAAACTCCATTTAGGGAGAAATGTTTAA | |
| | | | ATCTCTGGTATAAGTTTACTCCATACCAGAGTAAACTATATATTACTCTATATA | |
| | | | AGCAGTCTTGCAATAACTAATCACCACCATAGAAGAAAGAAACAGACTGCAA | |
| | | | GGAACAGAGTTGAGTGTCTGGAGTCATCAAAGGCATTAAAAACTCCAGTAAA | |
| | | | AGCTGGGGCCGTAGCAAAAATCATGAAAAACACTTCAACGTGTCCTTTCAATC | |
| | | | ATCCAATTAAATGTGGGTAGATTAATGAAAATGTATTACATCAATATTAACTC | |
| | | | ATCTATAGCACTTTGAGTATCTTTGTAGTTCATGATATCCTATCCTATAATGTG | |
| | | | GAGGTAAATGATTTTATATGCATTGGGGGTCATATATAAAACTTCAATGTAAT | |
| | | | TTCACTACAATAAATTGCCTTCCTTATTTGAAAGTAAAAATGTTCTTTTTTTAT | |
| | | | TGACATGTTAACTCCTTTCCCTCAATTAATAGAAATCCACTAAGAAAATCATT | |
| | | | CACATATTGGTTCACTCAACAAGCATTTATTAAATATATATTCACTATTCTAGA | |
| | | | CTAATAGCAAGACCGGGGATCTTGTTTAGGAAGAAGCAGTCCCTGTTCTCCAG | |
| | | | AATTTGCAAAACCATTAAAAAAGCACCTACTTTAAGCCATTTTTTTTCAGCAA | |
| | | | GGAGTCATTCTGCCAGAAAAATGTAGTACACAAATACAGGATAATATAACAA | |
| | | | ATGTAAAATTTCTCATTTCTAGTGAATTAAACTTTCCAGTAATTTTACATCTCA | |
| | | | CCTCATTCTTATGATGCTCAGTTTGCTTAATTATTGGCAAACATAATGGTAAAA | |
| | | | TGTTTGTACTGTATTAGAGCTTTACTGTTCGATTATTAAGATATTTATCCAGTA | |
| | | | TCTTAGAGATGCTGGACTTCAATTTTCCTTATTTTATCTTTTTAAAATAAAATA | |
| | | | TCATGCATGCTTCTGATTTGTTATAGAAA | |
| 46 | PTPRC | >NP_002829.3 | SEQ ID NO: 96 | SEQ ID NO: |
| | | receptor-type | >NM_002838.4 Homo sapiens protein tyrosine phosphatase, receptor type C (PTPRC), | 129 |
| | | tyrosine-protein | transcript variant 1, mRNA | NG_007730.1 |
| | | phosphatase C | AGAACAACTTTTTTGACTTCCTGCAAAGAGGACCCTTACAGTATTTTTGGAGA | |
| | | isoform 1 precursor | AGTTAGTAAAACCGAATCTGACATCATCACCTAGCAGTTCATGCAGCTAGCAA | |
| | | [Homo sapiens] | GTGGTTTGTTCTTAGGGTAACAGAGGAGGAAATTGTTCCTCGTCTGATAAGAC | |
| | | MTMYLWLKLLAF | AACAGTGGAGAAAGGACGCATGCTGTTTCTTAGGGACACGGCTGACTTCCAG | |
| | | GFAFLDTEVFVTG | ATATGACCATGTATTTGTGGCTTAAACTCTTGGCATTTGGCTTTGCCTTTCTGG | |
| | | QSPTPSPTGLTTAK | ACACAGAAGTATTTGTGACAGGGCAAAGCCCAACACCTTCCCCCACTGGATTG | |
| | | MWSVPLSSDPLPT | ACTACAGCAAAGATGCCCAGTGTTCCACTTTCAAGTGACCCCTTACCTACTCA | |
| | | HTTAFSPASEFERE | CACCACTGCATTCTCACCCGCAAGCACCTTTGAAAGAGAAAATGACTTCTCAG | |
| | | NDFSETTTSLSPDN | AGACCACAACTTCTCTTAGTCCAGACAATACTTCCACCCAAGTATCCCCGGAC | |
| | | TSTQVSPDSLDNA | TCTTTGGATAATGCTAGTGCTTTTAATACCACAGGTGTTTCATCAGTACAGACG | |
| | | SAFNTTGVSSVQT | CCTCACCTTCCCACGCACGCAGACTCGCAGACGCCCTCTGCTGGAACTGACAC | |
| | | PHLPTHADSQTPS | GCAGACATTCAGCGGCTCCGCCGCCAATGCAAAACTCAACCCTACCCCAGGC | |
| | | AGTDTQTFSGSAA | AGCAATGCTATCTCAGATGTCCCAGGAGAGAGGAGTACAGCCAGCACCTTTCC | |
| | | NAKLNPTPGSNAI | TACAGACCCAGTTTCCCCATTGACAACCACCCTCAGCCTTGCACACCACAGCT | |
| | | SDVPGERSTASIFP | CTGCTGCCTTACCTGCACGCACCTCCAACACCACCATCACAGCGAACACCTCA | |
| | | TDPVSPLTTTLSLA | GATGCCTACCTTAATGCCTCTGAAACAACCACTCTGAGCCCTTCTGGAAGCGC | |
| | | HHSSAALPARTSN | TGTCATTTCAACCACAACAATAGCTACTACTCCATCTAAGCCAACATGTGATG | |
| | | TTITANTSDAYLN | AAAAATATGCAAACATCACTGTGGATTACTTATATAACAAGGAAACTAAATTA | |
| | | ASETTTLSPSGSAV | TTTACAGCAAAGCTAAATGTTAATGAGAATGTGGAATGTGGAAACAATACTTG | |
| | | ISTTTIATTPSKPTC | CACAAACAATGAGGTGCATAACCTTACAGAATGTAAAAATGCGTCTGTTTCCA | |
| | | DEKYANITVDYLY | TATCTCATAATTCATGTACTGCTCCTGATAAGACATTAATATTAGATGTGCCAC | |
| | | NKETKLFTAKLNV | CAGGGGTTGAAAAGTTTCAGTTACATGATTGTACACAAGTTGAAAAAGCAGA | |
| | | NENVECGNNTCT | TACTACTATTTGTTTAAAATGGAAAAATATTGAAACCTTTACTTGTGATACAC | |
| | | NNEVHNLTECKN | AGAATATTACCTACAGATTTCAGTGTGGTAATATGATATTTGATAATAAAGAA | |
| | | ASVSISHNSCTAPD | ATTAAATTAGAAAACCTTGAACCCGAACATGAGTATAAGTGTGACTCAGAAA | |
| | | KTLILDVPPGVEK | TACTCTATAATAACCACAAGTTTACTAACGCAAGTAAAATTATTAAAACAGAT | |
| | | FQLHDCTQVEKA | TTTGGGAGTCCAGGAGAGCCTCAGATTATTTTTTGTAGAAGTGAAGCTGCACA | |
| | | DTTICLKWKNIET | TCAAGGAGTAATTACCTGGAATCCCCCTCAAAGATCATTTCATAATTTTACCCT | |
| | | FTCDTQNITYRFQ | CTGTTATATAAAAGAGACAGAAAAAGATTGCCTCAATCTGGATAAAAACCTG | |
| | | CGNMIFDNKEIKL | ATCAAATATGATTTGCAAAATTTAAAACCTTATACGAAATATGTTTTATCATTA | |
| | | ENLEPEHEYKCDS | CATGCCTACATCATTGCAAAAGTGCAACGTAATGGAAGTGCTGCAATGTGTCA | |
| | | EILYNNHKFTNAS | TTTCACAACTAAAAGTGCTCCTCCAAGCCAGGTCTGGAACATGACTGTCTCCA | |
| | | KIIKTDFGSPGEPQ | TGACATCAGATAATAGTATGCATGTCAAGTGTAGGCCTCCCAGGGACCGTAAT | |
| | | IIFCRSEAAHQGVI | GGCCCCCATGAACGTTACCATTTGGAAGTTGAAGCTGGAAATACTCTGGTTAG | |
| | | TWNPPQRSFHNFT | AAATGAGTCGCATAAGAATTGCGATTTCCGTGTAAAAGATCTTCAATATTCAA | |
| | | LCYIKETEKDCLN | CAGACTACACTTTTAAGGCCTATTTTCACAATGGAGACTATCCTGGAGAACCC | |
| | | LDKNLIKYDLQNL | TTTATTTTACATCATTCAACATCTTATAATTCTAAGGCACTGATAGCATTTCTG | |
| | | KPYTKYVLSLHAY | GCATTTCTGATTATTGTGACATCAATAGCCCTGCTTGTTGTTCTCTACAAAATC | |
| | | IIAKVQRNGSAAM | TATGATCTACATAAGAAAAGATCCTGCAATTTAGATGAACAGCAGGAGCTTGT | |
| | | CHFTTKSAPPSQV | TGAAAGGGATGATGAAAAACAACTGATGAATGTGGAGCCAATCCATGCAGAT | |
| | | WNMTVSMTSDNS | ATTTTGTTGGAAACTTATAAGAGGAAGATTGCTGATGAAGGAAGACTTTTTCT | |
| | | MHVKCRPPRDRN | GGCTGAATTTCAGAGCATCCCGCGGGTGTTCAGCAAGTTTCCTATAAAGGAAG | |
| | | GPFIERYHLEVEAG | CTCGAAAGCCCTTTAACCAGAATAAAAACCGTTATGTTGACATTCTTCCTTAT | |
| | | NTLVRNESHKNC | GATTATAACCGTGTTGAACTCTCTGAGATAAACGGAGATGCAGGGTCAAACTA | |
| | | DFRVKDLQYSTD | CATAAATGCCAGCTATATTGATGGTTTCAAAGAACCCAGGAAATACATTGCTG | |
| | | YTFKAYFHNGDY | CACAAGGTCCCAGGGATGAAACTGTTGATGATTTCTGGAGGATGATTTGGGAA | |
| | | PGEPFILHHSTSYN | CAGAAAGCCACAGTTATTGTCATGGTCACTCGATGTGAAGAAGGAAACAGGA | |
| | | SKALIAFLAFLIIVT | ACAAGTGTGCAGAATACTGGCCGTCAATGGAAGAGGGCACTCGGGCTTTTGG | |
| | | SIALLVVLYKIYDL | AGATGTTGTTGTAAAGATCAACCAGCACAAAAGATGTCCAGATTACATCATTC | |
| | | HKKRSCNLDEQQ | AGAAATTGAACATTGTAAATAAAAAAGAAAAAGCAACTGGAAGAGAGGTGA | |
| | | ELVERDDEKQLM | CTCACATTCAGTTCACCAGCTGGCCAGACCACGGGGTGCCTGAGGATCCTCAC | |
| | | NVEPIHADILLETY | TTGCTCCTCAAACTGAGAAGGAGAGTGAATGCCTTCAGCAATTTCTTCAGTGG | |
| | | KRKIADEGRLFLA | TCCCATTGTGGTGCACTGCAGTGCTGGTGTTGGGCGCACAGGAACCTATATCG | |
| | | EFQSIPRVESKEPIK | GAATTGATGCCATGCTAGAAGGCCTGGAAGCCGAGAACAAAGTGGATGTTTA | |
| | | EARKPFNQNKNR | TGGTTATGTTGTCAAGCTAAGGCGACAGAGATGCCTGATGGTTCAAGTAGAGG | |
| | | YVDILPYDYNRVE | CCCAGTACATCTTGATCCATCAGGCTTTGGTGGAATACAATCAGTTTGGAGAA | |
| | | LSEINGDAGSNYI | ACAGAAGTGAATTTGTCTGAATTACATCCATATCTACATAACATGAAGAAAAG | |
| | | NASYIDGFKEPRK | GGATCCACCCAGTGAGCCGTCTCCACTAGAGGCTGAATTCCAGAGACTTCCTT | |
| | | YIAAQGPRDETVD | CATATAGGAGCTGGAGGACACAGCACATTGGAAATCAAGAAGAAAATAAAA | |
| | | DFWRMIWEQKAT | GTAAAAACAGGAATTCTAATGTCATCCCATATGACTATAACAGAGTGCCACTT | |
| | | VIVMVTRCEEGNR | AAACATGAGCTGGAAATGAGTAAAGAGAGTGAGCATGATTCAGATGAATCCT | |
| | | NKCAEYWPSMEE | CTGATGATGACAGTGATTCAGAGGAACCAAGCAAATACATCAATGCATCTTTT | |
| | | GTRAFGDVVVKIN | ATAATGAGCTACTGGAAACCTGAAGTGATGATTGCTGCTCAGGGACCACTGA | |
| | | QHKRCPDYIIQKL | AGGAGACCATTGGTGACTTTTGGCAGATGATCTTCCAAAGAAAAGTCAAAGTT | |
| | | NIVNKKEKATGRE | ATTGTTATGCTGACAGAACTGAAACATGGAGACCAGGAAATCTGTGCTCAGTA | |
| | | VTHIQFTSWPDHG | CTGGGGAGAAGGAAAGCAAACATATGGAGATATTGAAGTTGACCTGAAAGAC | |
| | | VPEDPHLLLKLRR | ACAGACAAATCTTCAACTTATACCCTTCGTGTCTTTGAACTGAGACATTCCAA | |
| | | RVNAFSNFFSGPIV | GAGGAAAGACTCTCGAACTGTGTACCAGTACCAATATACAAACTGGAGTGTG | |
| | | VHCSAGVGRTGT | GAGCAGCTTCCTGCAGAACCCAAGGAATTAATCTCTATGATTCAGGTCGTCAA | |
| | | YIGIDAMLEGLEA | ACAAAAACTTCCCCAGAAGAATTCCTCTGAAGGGAACAAGCATCACAAGAGT | |
| | | ENKVDVYGYVVK | ACACCTCTACTCATTCACTGCAGGGATGGATCTCAGCAAACGGGAATATTTTG | |
| | | LRRQRCLMVQVE | TGCTTTGTTAAATCTCTTAGAAAGTGCGGAAACAGAAGAGGTAGTGGATATTT | |
| | | AQYILIHQALVEY | TTCAAGTGGTAAAAGCTCTACGCAAAGCTAGGCCAGGCATGGTTTCCACATTC | |
| | | NQFGETEVNLSEL | GAGCAATATCAATTCCTATATGACGTCATTGCCAGCACCTACCCTGCTCAGAA | |
| | | HPYLHNMKKRDP | TGGACAAGTAAAGAAAAACAACCATCAAGAAGATAAAATTGAATTTGATAAT | |
| | | PSEPSPLEAEFQRL | GAAGTGGACAAAGTAAAGCAGGATGCTAATTGTGTTAATCCACTTGGTGCCCC | |
| | | PSYRSWRTQHIGN | AGAAAAGCTCCCTGAAGCAAAGGAACAGGCTGAAGGTTCTGAACCCACGAGT | |
| | | QEENKSKNRNSN | GGCACTGAGGGGCCAGAACATTCTGTCAATGGTCCTGCAAGTCCAGCTTTAAA | |
| | | VIPYDYNRVPLKH | TCAAGGTTCATAGGAAAAGACATAAATGAGGAAACTCCAAACCTCCTGTTAG | |
| | | ELEMSKESEHDSD | CTGTTATTTCTATTTTTGTAGAAGTAGGAAGTGAAAATAGGTATACAGTGGAT | |
| | | ESSDDDSDSEEPS | TAATTAAATGCAGCGAACCAATATTTGTAGAAGGGTTATATTTTACTACTGTG | |
| | | KYINASHMSYWK | GAAAAATATTTAAGATAGTTTTGCCAGAACAGTTTGTACAGACGTATGCTTAT | |
| | | PEVMIAAQGPLKE | TTTAAAATTTTATCTCTTATTCAGTAAAAAACAACTTCTTTGTAATCGTTATGT | |
| | | TIGDFWQMIFQRK | GTGTATATGTATGTGTGTATGGGTGTGTGTTTGTGTGAGAGACAGAGAAAGAG | |
| | | VKVIVMLTELKHG | AGAGAATTCTTTCAAGTGAATCTAAAAGCTTTTGCTTTTCCTTTGTTTTTATGA | |
| | | DQEICAQYWGEG | AGAAAAAATACATTTTATATTAGAAGTGTTAACTTAGCTTGAAGGATCTGTTT | |
| | | KQTYGDIEVDLKD | TTAAAAATCATAAACTGTGTGCAGACTCAATAAAATCATGTACATTTCTGAAA | |
| | | TDKSSTYTLRVFE | TGACCTCAAGATGTCCTCCTTGTTCTACTCATATATATCTATCTTATATAGTTT | |
| | | LRHSKRKDSRTVY | ACTATTTTACTTCTAGAGATAGTACATAAAGGTGGTATGTGTGTGTATGCTACT | |
| | | QYQYTNWSVEQL | ACAAAAAAGTTGTTAACTAAATTAACATTGGGAAATCTTATATTCCATATATT | |
| | | PAEPKELISMIQVV | AGCATTTAGTCCAATGTCTTTTTAAGCTTATTTAATTAAAAAATTTCCAGTGAG | |
| | | KQKLPQKNSSEGN | CTTATCATGCTGTCTTTACATGGGGTTTTCAATTTTGCATGCTCGATTATTCCCT | |
| | | KHHKSTPLLIHCR | GTACAATATTTAAAATTTATTGCTTGATACTTTTGACAACAAATTAGGTTTTGT | |
| | | DGSQQTGIFCALL | ACAATTGAACTTAAATAAATGTCATTAAAATAAATAAATGCAATATGTATTAA | |
| | | NLLESAETEEVVD | TATTCATTGTATAAAAATAGAAGAATACAAACATATTTGTTAAATATTTACAT | |
| | | IFQVVKALRKARP | ATGAAATTTAATATAGCTATTTTTATGGAATTTTTCATTGATATGAAAAATATG | |
| | | GMVSTFEQYQFLY | ATATTGCATATGCATAGTTCCCATGTTAAATCCCATTCATAACTTTCATTAAAG | |
| | | DVIASTYPAQNGQ | CATTTACTTTGAATTTCTCCAATGCTTAGAATGTTTTTACCAGGAATGGATGTC | |
| | | VKKNNHQEDKIEF | GCTAATCATAATAAAATTCAACCATTATTTTTTTCTTGTTTATAATACATTGTG | |
| | | DNEVDKVKQDAN | TTATATGTTCAAATATGAAATGTGTATGCACCTATTGAAATATGTTTAATGCAT | |
| | | CVNPLGAPEKLPE | TTATTAACATTTGCAGGACACTTTTACAGGCCCCAATTATCCAATAGTCTAATA | |
| | | AKEQAEGSEPTSG | ATTGTTTAAGATCTAGAAAAAAAAAATCAAGAATAGTGGTATTTTTCATGAAG | |
| | | TEGPEHSVNGPAS | TAATAAAAACTCGTTTTGGTGAA | |
| | | PALNQGS | | |
| 47 | CIITA | >NP_001273331.1 | SEQ ID NO: 97 | SEQ ID NO: |
| | | MHC class II | >NM_001286402.1 Homo sapiens class II major histocompatibility complex | 130 |
| | | transactivator | transactivator (CIITA), transcript variant 1, mRNA | NG_009628.1 |
| | | isoform 1 [Homo | GGTTAGTGATGAGGCTAGTGATGAGGCTGTGTGCTTCTGAGCTGGGCATCCGA | |
| | | sapiens] | AGGCATCCTTGGGGAAGCTGAGGGCACGAGGAGGGGCTGCCAGACTCCGGGA | |
| | | MRCLAPRPAGSYL | GCTGCTGCCTGGCTGGGATTCCTACACAATGCGTTGCCTGGCTCCACGCCCTG | |
| | | SEPQGSSQCATME | CTGGGTCCTACCTGTCAGAGCCCCAAGGCAGCTCACAGTGTGCCACCATGGAG | |
| | | LGPLEGGYLELLN | TTGGGGCCCCTAGAAGGTGGCTACCTGGAGCTTCTTAACAGCGATGCTGACCC | |
| | | SDADPLCLYHFYD | CCTGTGCCTCTACCACTTCTATGACCAGATGGACCTGGCTGGAGAAGAAGAGA | |
| | | QMDLAGEEEIELY | TTGAGCTCTACTCAGAACCCGACACAGACACCATCAACTGCGACCAGTTCAGC | |
| | | SEPDTDTINCDQFS | AGGCTGTTGTGTGACATGGAAGGTGATGAAGAGACCAGGGAGGCTTATGCCA | |
| | | RLLCDMEGDEETR | ATATCGCGGAACTGGACCAGTATGTCTTCCAGGACTCCCAGCTGGAGGGCCTG | |
| | | EAYANIAELDQYV | AGCAAGGACATTTTCATAGAGCACATAGGACCAGATGAAGTGATCGGTGAGA | |
| | | FQDSQLEGLSKDIF | GTATGGAGATGCCAGCAGAAGTTGGGCAGAAAAGTCAGAAAAGACCCTTCCC | |
| | | IEHIGPDEVIGESM | AGAGGAGCTTCCGGCAGACCTGAAGCACTGGAAGCCAGCTGAGCCCCCCACT | |
| | | EMPAEVGQKSQK | GTGGTGACTGGCAGTCTCCTAGTGGGACCAGTGAGCGACTGCTCCACCCTGCC | |
| | | RPFPEELPADLKH | CTGCCTGCCACTGCCTGCGCTGTTCAACCAGGAGCCAGCCTCCGGCCAGATGC | |
| | | WKPAEPPTVVTGS | GCCTGGAGAAAACCGACCAGATTCCCATGCCTTTCTCCAGTTCCTCGTTGAGC | |
| | | LLVGPVSDCSTLP | TGCCTGAATCTCCCTGAGGGACCCATCCAGTTTGTCCCCACCATCTCCACTCTG | |
| | | CLPLPALFNQEPA | CCCCATGGGCTCTGGCAAATCTCTGAGGCTGGAACAGGGGTCTCCAGTATATT | |
| | | SGQMRLEKTDQIP | CATCTACCATGGTGAGGTGCCCCAGGCCAGCCAAGTACCCCCTCCCAGTGGAT | |
| | | MPFSSSSLSCLNLP | TCACTGTCCACGGCCTCCCAACATCTCCAGACCGGCCAGGCTCCACCAGCCCC | |
| | | EGPIQFVPTISTLP | TTCGCTCCATCAGCCACTGACCTGCCCAGCATGCCTGAACCTGCCCTGACCTC | |
| | | HGLWQISEAGTGV | CCGAGCAAACATGACAGAGCACAAGACGTCCCCCACCCAATGCCCGGCAGCT | |
| | | SSIFIYHGEVPQAS | GGAGAGGTCTCCAACAAGCTTCCAAAATGGCCTGAGCCGGTGGAGCAGTTCT | |
| | | QVPPPSGFTVHGL | ACCGCTCACTGCAGGACACGTATGGTGCCGAGCCCGCAGGCCCGGATGGCAT | |
| | | PTSPDRPGSTSPFA | CCTAGTGGAGGTGGATCTGGTGCAGGCCAGGCTGGAGAGGAGCAGCAGCAAG | |
| | | PSATDLPSMPEPA | AGCCTGGAGCGGGAACTGGCCACCCCGGACTGGGCAGAACGGCAGCTGGCCC | |
| | | LTSRANMTEHKTS | AAGGAGGCCTGGCTGAGGTGCTGTTGGCTGCCAAGGAGCACCGGCGGCCGCG | |
| | | PTQCPAAGEVSNK | TGAGACACGAGTGATTGCTGTGCTGGGCAAAGCTGGTCAGGGCAAGAGCTAT | |
| | | LPKWPEPVEQFYR | TGGGCTGGGGCAGTGAGCCGGGCCTGGGCTTGTGGCCGGCTTCCCCAGTACGA | |
| | | SLQDTYGAEPAGP | CTTTGTCTTCTCTGTCCCCTGCCATTGCTTGAACCGTCCGGGGGATGCCTATGG | |
| | | DGILVEVDLVQAR | CCTGCAGGATCTGCTCTTCTCCCTGGGCCCACAGCCACTCGTGGCGGCCGATG | |
| | | LERSSSKSLERELA | AGGTTTTCAGCCACATCTTGAAGAGACCTGACCGCGTTCTGCTCATCCTAGAC | |
| | | TPDWAERQLAQG | GGCTTCGAGGAGCTGGAAGCGCAAGATGGCTTCCTGCACAGCACGTGCGGAC | |
| | | GLAEVLLAAKEH | CGGCACCGGCGGAGCCCTGCTCCCTCCGGGGGCTGCTGGCCGGCCTTTTCCAG | |
| | | RRPRETRVIAVLG | AAGAAGCTGCTCCGAGGTTGCACCCTCCTCCTCACAGCCCGGCCCCGGGGCCG | |
| | | KAGQGKSYWAGA | CCTGGTCCAGAGCCTGAGCAAGGCCGACGCCCTATTTGAGCTGTCCGGCTTCT | |
| | | VSRAWACGRLPQ | CCATGGAGCAGGCCCAGGCATACGTGATGCGCTACTTTGAGAGCTCAGGGAT | |
| | | YDFVFSVPCHCLN | GACAGAGCACCAAGACAGAGCCCTGACGCTCCTCCGGGACCGGCCACTTCTTC | |
| | | RPGDAYGLQDLLF | TCAGTCACAGCCACAGCCCTACTTTGTGCCGGGCAGTGTGCCAGCTCTCAGAG | |
| | | SLGPQPLVAADEV | GCCCTGCTGGAGCTTGGGGAGGACGCCAAGCTGCCCTCCACGCTCACGGGACT | |
| | | FSHILKRPDRVLLI | CTATGTCGGCCTGCTGGGCCGTGCAGCCCTCGACAGCCCCCCCGGGGCCCTGG | |
| | | LDGFEELEAQDGF | CAGAGCTGGCCAAGCTGGCCTGGGAGCTGGGCCGCAGACATCAAAGTACCCT | |
| | | LHSTCGPAPAEPC | ACAGGAGGACCAGTTCCCATCCGCAGACGTGAGGACCTGGGCGATGGCCAAA | |
| | | SLRGLLAGLFQKK | GGCTTAGTCCAACACCCACCGCGGGCCGCAGAGTCCGAGCTGGCCTTCCCCAG | |
| | | LLRGCTLLLTARP | CTTCCTCCTGCAATGCTTCCTGGGGGCCCTGTGGCTGGCTCTGAGTGGCGAAA | |
| | | RGRLVQSLSKADA | TCAAGGACAAGGAGCTCCCGCAGTACCTAGCATTGACCCCAAGGAAGAAGAG | |
| | | LFELSGFSMEQAQ | GCCCTATGACAACTGGCTGGAGGGCGTGCCACGCTTTCTGGCTGGGCTGATCT | |
| | | AYVMRYFESSGM | TCCAGCCTCCCGCCCGCTGCCTGGGAGCCCTACTCGGGCCATCGGCGGCTGCC | |
| | | TEHQDRALTLLRD | TCGGTGGACAGGAAGCAGAAGGTGCTTGCGAGGTACCTGAAGCGGCTGCAGC | |
| | | RPLLLSHSHSPTLC | CGGGGACACTGCGGGCGCGGCAGCTGCTGGAGCTGCTGCACTGCGCCCACGA | |
| | | RAVCQLSEALLEL | GGCCGAGGAGGCTGGAATTTGGCAGCACGTGGTACAGGAGCTCCCCGGCCGC | |
| | | GEDAKLPSTLTGL | CTCTCTTTTCTGGGCACCCGCCTCACGCCTCCTGATGCACATGTACTGGGCAAG | |
| | | YVGLLGRAALDSP | GCCTTGGAGGCGGCGGGCCAAGACTTCTCCCTGGACCTCCGCAGCACTGGCAT | |
| | | PGALAELAKLAW | TTGCCCCTCTGGATTGGGGAGCCTCGTGGGACTCAGCTGTGTCACCCGTTTCA | |
| | | ELGRRHQSTLQED | GGGCTGCCTTGAGCGACACGGTGGCGCTGTGGGAGTCCCTGCAGCAGCATGG | |
| | | QFPSADVRTWAM | GGAGACCAAGCTACTTCAGGCAGCAGAGGAGAAGTTCACCATCGAGCCTTTC | |
| | | AKGLVQHPPRAA | AAAGCCAAGTCCCTGAAGGATGTGGAAGACCTGGGAAAGCTTGTGCAGACTC | |
| | | ESELAFPSFLLQCF | AGAGGACGAGAAGTTCCTCGGAAGACACAGCTGGGGAGCTCCCTGCTGTTCG | |
| | | LGALWLALSGEIK | GGACCTAAAGAAACTGGAGTTTGCGCTGGGCCCTGTCTCAGGCCCCCAGGCTT | |
| | | DKELPQYLALTPR | TCCCCAAACTGGTGCGGATCCTCACGGCCTTTTCCTCCCTGCAGCATCTGGACC | |
| | | KKRPYDNWLEGV | TGGATGCGCTGAGTGAGAACAAGATCGGGGACGAGGGTGTCTCGCAGCTCTC | |
| | | PRFLAGLIFQPPAR | AGCCACCTTCCCCCAGCTGAAGTCCTTGGAAACCCTCAATCTGTCCCAGAACA | |
| | | CLGALLGPSAAAS | ACATCACTGACCTGGGTGCCTACAAACTCGCCGAGGCCCTGCCTTCGCTCGCT | |
| | | VDRKQKVLARYL | GCATCCCTGCTCAGGCTAAGCTTGTACAATAACTGCATCTGCGACGTGGGAGC | |
| | | KRLQPGTLRARQL | CGAGAGCTTGGCTCGTGTGCTTCCGGACATGGTGTCCCTCCGGGTGATGGACG | |
| | | LELLHCAHEAEEA | TCCAGTACAACAAGTTCACGGCTGCCGGGGCCCAGCAGCTCGCTGCCAGCCTT | |
| | | GIWQHVVQELPG | CGGAGGTGTCCTCATGTGGAGACGCTGGCGATGTGGACGCCCACCATCCCATT | |
| | | RLSFLGTRLTPPD | CAGTGTCCAGGAACACCTGCAACAACAGGATTCACGGATCAGCCTGAGATGA | |
| | | AHVLGKALEAAG | TCCCAGCTGTGCTCTGGACAGGCATGTTCTCTGAGGACACTAACCACGCTGGA | |
| | | QDFSLDLRSTGICP | CCTTGAACTGGGTACTTGTGGACACAGCTCTTCTCCAGGCTGTATCCCATGAG | |
| | | SGLGSLVGLSCVT | CCTCAGCATCCTGGCACCCGGCCCCTGCTGGTTCAGGGTTGGCCCCTGCCCGG | |
| | | RFRAALSDTVAL | CTGCGGAATGAACCACATCTTGCTCTGCTGACAGACACAGGCCCGGCTCCAGG | |
| | | WESLQQHGETKL | CTCCTTTAGCGCCCAGTTGGGTGGATGCCTGGTGGCAGCTGCGGTCCACCCAG | |
| | | LQAAEEKFTIEPFK | GAGCCCCGAGGCCTTCTCTGAAGGACATTGCGGACAGCCACGGCCAGGCCAG | |
| | | AKSLKDVEDLGK | AGGGAGTGACAGAGGCAGCCCCATTCTGCCTGCCCAGGCCCCTGCCACCCTGG | |
| | | LVQTQRTRSSSED | GGAGAAAGTACTTCTTTTTTTTTATTTTTAGACAGAGTCTCACTGTTGCCCAGG | |
| | | TAGELPAVRDLKK | CTGGCGTGCAGTGGTGCGATCTGGGTTCACTGCAACCTCCGCCTCTTGGGTTC | |
| | | LEFALGPVSGPQA | AAGCGATTCTTCTGCTTCAGCCTCCCGAGTAGCTGGGACTACAGGCACCCACC | |
| | | FPKLVRILTAFSSL | ATCATGTCTGGCTAATTTTTCATTTTTAGTAGAGACAGGGTTTTGCCATGTTGG | |
| | | QHLDLDALSENKI | CCAGGCTGGTCTCAAACTCTTGACCTCAGGTGATCCACCCACCTCAGCCTCCC | |
| | | GDEGVSQLSATFP | AAAGTGCTGGGATTACAAGCGTGAGCCACTGCACCGGGCCACAGAGAAAGTA | |
| | | QLKSLETLNLSQN | CTTCTCCACCCTGCTCTCCGACCAGACACCTTGACAGGGCACACCGGGCACTC | |
| | | NITDLGAYKLAEA | AGAAGACACTGATGGGCAACCCCCAGCCTGCTAATTCCCCAGATTGCAACAG | |
| | | LPSLAASLLRLSLY | GCTGGGCTTCAGTGGCAGCTGCTTTTGTCTATGGGACTCAATGCACTGACATT | |
| | | NNCICDVGAESLA | GTTGGCCAAAGCCAAAGCTAGGCCTGGCCAGATGCACCAGCCCTTAGCAGGG | |
| | | RVLPDMVSLRVM | AAACAGCTAATGGGACACTAATGGGGCGGTGAGAGGGGAACAGACTGGAAG | |
| | | DVQYNKFTAAGA | CACAGCTTCATTTCCTGTGTCTTTTTTCACTACATTATAAATGTCTCTTTAATGT | |
| | | QQLAASLRRCPHV | CACAGGCAGGTCCAGGGTTTGAGTTCATACCCTGTTACCATTTTGGGGTACCC | |
| | | ETLAMWTPTIPFS | ACTGCTCTGGTTATCTAATATGTAACAAGCCACCCCAAATCATAGTGGCTTAA | |
| | | VQEHLQQQDSRIS | AACAACACTCACATTTA | |
| | | LR | | |
| 48 | HLA-DOA | >NP_002110.1 HLA | SEQ ID NO: 98 | SEQ ID NO: |
| | | class II | >NM_002119.3 Homo sapiens major histocompatibility complex, class II, DO alpha | 131 |
| | | histocompatibility | (HLA-DOA), mRNA | NG_012007.1 |
| | | antigen, DO alpha | CTTCTTCTTTACCTCCGCCTTGTTCCTGTCCTCACCACACGGACTGAGACTGAT | |
| | | chain precursor | TTGATTAAAGCACCAGAGTGTAATGGCCCTCAGAGCAGGGCTGGTCCTGGGGT | |
| | | [Homo sapiens] | TCCACACCCTGATGACCCTCCTGAGCCCGCAGGAGGCAGGGGCCACCAAGGC | |
| | | MALRAGLVLGFH | TGACCACATGGGCTCCTACGGACCCGCCTTCTACCAGTCTTACGGCGCCTCGG | |
| | | TLMTLLSPQEAGA | GCCAGTTCACCCATGAATTTGATGAGGAACAGCTGTTCTCTGTGGACCTGAAG | |
| | | TKADHMGSYGPA | AAAAGCGAGGCCGTGTGGCGTCTGCCTGAGTTTGGTGACTTTGCCCGCTTTGA | |
| | | FYQSYGASGQFTH | CCCGCAGGGCGGGCTGGCCGGCATCGCCGCAATCAAAGCCCATCTGGACATC | |
| | | EFDEEQLFSVDLK | CTGGTGGAGCGCTCCAACCGCAGCAGAGCCATCAACGTGCCTCCACGGGTGA | |
| | | KSEAVWRLPEFGD | CCGTGCTCCCCAAGTCTCGGGTGGAGCTGGGCCAGCCCAACATCCTCATCTGC | |
| | | FARFDPQGGLAGI | ATCGTGGACAACATCTTCCCCCCTGTGATCAATATCACCTGGCTGCGCAACGG | |
| | | AMKAHLDILVER | CCAAACTGTCACTGAGGGAGTGGCCCAGACCAGCTTCTATTCCCAGCCTGACC | |
| | | SNRSRAINVPPRV | ATTTGTTCCGCAAGTTCCACTACCTGCCCTTCGTGCCCTCAGCCGAGGACGTCT | |
| | | TVLPKSRVELGQP | ATGACTGCCAGGTGGAGCACTGGGGCCTGGATGCGCCACTCCTCAGGCATTGG | |
| | | NILICIVDNIFPPVI | GAGCTCCAGGTGCCTATTCCACCACCAGATGCCATGGAGACCCTGGTCTGTGC | |
| | | NITWLRNGQTVTE | CCTGGGCCTGGCCATCGGCCTGGTGGGCTTCCTCGTGGGCACCGTCCTCATCA | |
| | | GVAQTSFYSQPDH | TCATGGGCACATATGTGTCCAGTGTCCCCAGGTAATGATCCTTCTGAGAGAAA | |
| | | LFRKFHYLPFVPS | TGACTTGTGGGAGACACCCTGCAGATCCTCATGGGTTTGTGACAGCCCCTGCG | |
| | | AEDVYDCQVEHW | TGCTCAGTGCCCTTTAAGTGCATCCCGCTGTGCTGACTTTGAGTGGGATCAAC | |
| | | GLDAPLLRHWEL | ATCTGTCCTACGGGTCCCCTCTTTTTTGGCCCCAGTATTCATGGCAGGGTTTGT | |
| | | QVPIPPPDAMETL | TGGACACCTACTAGCTTCCCTTCCCATTCAACACACACACACATTCTTGCTCTA | |
| | | VCALGLAIGLVGF | CCCAAAGCTCTGGCTGGCAGCACTAAATGCTTTGGTGGTGTTTGCACTGTGTC | |
| | | LVGTVLIIMGTYV | CTTTCCAGGCCTTGGCCAGTTCTTCCAGGGGTGAGGCATGTGGTGCTGGGGAT | |
| | | SSVPR | TGGCAGCCATCCTGGGGCCCACACAGGTGTGTCTTGCTCCATTTGGCCCATTG | |
| | | | TGTGTTACTTTGTGAATGAGCCATTTCACATGGACTTCATGAAATTTGCCTCCT | |
| | | | GAGTTCAGGTTTACCCTGAAAGGGATGCAGATTATCCTGTTCCTCACGACCCC | |
| | | | CTCAGCTAACAACAGTTCTGAAGGGTGCTGGGACAGGACAGGCTCATGGGGA | |
| | | | CTCCACTCCTGCCTGGGTTTACTCTGTATGAAGAGGCCACTGGTATCCTGCCAT | |
| | | | GATGTTATCTCCTTTTTCTACTTTTCCCTAGAGTCCCATGCATGATAAAGAGAG | |
| | | | GCCCAAGGCTTGGATAAGGTGGCCACTTCCCTCAGTGGAGTCAGTCATGTTAG | |
| | | | GTAGGAGGTGGTAGAGTCGGTCTGCGAGGTATCTCGTAAGAGGGGAGGTCCA | |
| | | | CCTAGACACACTCTAAATATGTGGCCTAGAAGATTTTGGTCTACTTTTCTGTGA | |
| | | | ACAGAATTTAAAACATACAAAGAGATAAATCACCATACCACATAGTTTATGTC | |
| | | | AGGACCAAAATGAGCAATACAGATTACGGTTTTCAAACCAGAATGCACATAA | |
| | | | GAACTGCTTGGGATCCTTTTAAAAGTACAGGCATTGGCCTGGTGCAGTGGCTC | |
| | | | ATTCCTGTAATCCCAGCACTTTGGGAGGCCAAGGGGACAGGACTGCTTGAGGC | |
| | | | CAAGAGGTGGAAACCATCTTGGGCTACATAGAGAGACCCCATCTCTACAAAG | |
| | | | AAAGATTTAAAAATTAACCAGGCATGGTGGCTCGCACCTGTATTCCCAGCCAC | |
| | | | TGGGGAGGCTGAGGCCGGAGGAGTGCTTGAGCCCAGGAGTTCAAGGCTGCAG | |
| | | | TGAGCCAAGATTGCGCCACTGCACTCCAGCCTAGGTGACAGAGTGAGACCCT | |
| | | | GTCTCTAAATAAATAAATAAATAAAATATAAAAATAACAGTCATCACCCAGA | |
| | | | CCTACTGAATTAGAATCTCGGGAGTGCAGGGGGCAGCAACAGGGAGGCTGTC | |
| | | | TTTTCTGAGATGGGGTCTCACTCTGTCACCAGGCTGGAGTGCCATGGCATGAT | |
| | | | CTCAGCTCACTGCAACCTCCACCTCCTGAGTTCAAGCCATTCTCCTGCCTCAGC | |
| | | | CTCCTGAGTAGCTGGGACTACAGGTGTGCGCCACTACACTCAGCTAATTTTTG | |
| | | | TATTTTAAGTAGAGACGGGGTTTCATCATGTTGGCCAGGATGGCCTCCATCTC | |
| | | | TTGACCTCGTGATCCACCCACCTTCCCTCCCAAAGTACTGGAATTACAGGCAT | |
| | | | TAGCCACTGTGCCCAGCCGAGGCTGTCATTTTTAACCGGCTCTGGATGACTCT | |
| | | | GATGCAGCCATCCTGGACCTTGGCTGTGGTCTGGTAACTGGAACCCAGTGACG | |
| | | | TAATCAGGTGCCATCGGGGGTCATGGGAAAGGGGGATCCCCAAGGTCTGAGG | |
| | | | TGGACTAGGAAGGCTTTCTGAAGAACCTGGGTCTGTTAGGGCATCAGCCAATC | |
| | | | AAGGTACAAGTAAATAGAGGCAAAATGAGGGTTTGAACTGTGAGCAGTTGGT | |
| | | | CCTGGAAAAGAAAGAAACCAAGAGATTATGGGGACTCAATGGGCTTCTTAAG | |
| | | | AGAGAATAAGTTGAAATCAATGACCAGAAGACCCTGATGGAAGTGGAGGAGA | |
| | | | ATCATCTCAGGCAAACTTTTTGTGTGCCAGTAACAGAAACCCTCTTTGTGTGAT | |
| | | | CACATGCAAAGTATAGGATATTTGCAATATAGCCATGGGGAGGAGTGCAGGG | |
| | | | CCCAAGGGTAGATTTTAGCCAGGCCTCCCAGGAACAGAACTCGGATCCGAAA | |
| | | | AGCCCAGAGAAGCTAGAGCTGCCCCTCCAACACTCTCGGATCCACATGGTCTG | |
| | | | TGTTCTCTAGACCCCCCTGCATGTTAGCGGTGTTCTCTCTCTGTGGACTGACTG | |
| | | | TCCTTCTCAGTGAACATGTCCACCCGACAGCTCCTGAGTTTATATCATCTCAAC | |
| | | | CCTCACAACCCACAGAGGCTGTGTCTCCTAGTCACAGCTTTAAATTACTGGAA | |
| | | | AAATAAATGACTGGCCAAACTTGGAGCAGGTGTCCATCCCAGCCCTGTGTAGT | |
| | | | TAGAGCAGGAATCAAGATCTCAACACAAATGTGGCTGCCAAGCACTCAGCCC | |
| | | | CGGGGCGAGGGGTCAAGTTCTTCTCAGAGAAAGAGGAATAAGTTGGTTCTCA | |
| | | | GAAGACATCACAAGATACGTGTGTACCCAACAATCTCTGATCTCTGCTGATCT | |
| | | | TTTGCTTAGACGTTAACTTGATGCATCATTGGAAAGGTGTTTCTCTCATCTCTG | |
| | | | TCCTAAGGCTTGATAAAGTCATTAAAATTGTGTTCTTTTGACTAAA | |
| 49 | HLA-DMA | >NP_006111.2 HLA | SEQ ID NO: 99 | SEQ ID NO: |
| | | class II | >NM_006120.3 Homo sapiens major histocompatibility complex, class II, DM alpha | 132 |
| | | histocompatibility | (HLA-DMA), mRNA | NG_012006.1 |
| | | antigen, DM alpha | GATCTAAGGCCACCCTCTCGGGGAGGGAGTTGGGGAAGCTGGGTYGGCTGGG | |
| | | chain precursor | TTGGTAGCTCCTACCTACTGTGTGGCAAGAAGGTATGGGTCATGAACAGAACC | |
| | | Homo sapiens] | AAGGAGCTGCGCTGCTACAGATGTTACCACTTCTGTGGCTGCTACCCCACTCC | |
| | | MGHEQNQGAALL | TGGGCCGTCCCTGAAGCTCCTACTCCAATGTGGCCAGATGACCTGCAAAACCA | |
| | | QMLPLLWLLPHS | CACATTCCTGCACACAGTGTACTGCCAGGATGGGAGTCCCAGTGTGGGACTCT | |
| | | WAVPEAPTPMWP | CTGAGGCCTACGACGAGGACCAGCTTTTCTTCTTCGACTTTTCCCAGAACACTC | |
| | | DDLQNHTFLHTV | GGGTGCCTCGCCTGCCCGAATTTGCTGACTGGGCTCAGGAACAGGGAGATGCT | |
| | | YCQDGSPSVGLSE | CCTGCCATTTTATTTGACAAAGAGTTCTGCGAGTGGATGATCCAGCAAATAGG | |
| | | AYDEDQLFFFDFS | GCCAAAACTTGATGGGAAAATCCCGGTGTCCAGAGGGTTTCCTATCGCTGAAG | |
| | | QNTRVPRLPEFAD | TGTTCACGCTGAAGCCCCTGGAGTTTGGCAAGCCCAACACTTTGGTCTGTTTTG | |
| | | WAQEQGDAPAILF | TCAGTAATCTCTTCCCACCCATGCTGACAGTGAACTGGCAGCATCATTCCGTC | |
| | | DKEFCEWMIQQIG | CCTGTGGAAGGATTTGGGCCTACTTTTGTCTCAGCTGTCGATGGACTCAGCTTC | |
| | | PKLDGKIPVSRGFP | CAGGCCTTTTCTTACTTAAACTTCACACCAGAACCTTCTGACATTTTCTCCTGC | |
| | | IAEVFTLKPLEFGK | ATTGTGACTCACGAAATTGACCGCTACACAGCAATTGCCTATTGGGTACCCCG | |
| | | PNTLVCFVSNLFPP | GAACGCACTGCCCTCAGATCTGCTGGAGAATGTGCTGTGTGGCGTGGCCTTTG | |
| | | MLTVNWQHHSVP | GCCTGGGTGTGCTGGGCATCATCGTGGGCATTGTTCTCATCATCTACTTCCGGA | |
| | | VEGFGPTFVSAVD | AGCCTTGCTCAGGTGACTGATTCTTCCAGACCAGAGTTTGATGCCAGCAGCTT | |
| | | GLSFQAFSYLNFT | CGGCCATCCAAACAGAGGATGCTCAGATTTCTCACATCCTGCCCAGGATCTCC | |
| | | PEPSDIFSCIVTHEI | TCTTAGGGTAGAAGTCTCTGGGACATCCCTGGGGTGTGTGTGTAGATTTCCCA | |
| | | DRYTAIAYWVPR | CCTGGGGACTCTGCTGTCCCTGGGCTTGCATCCCAGGGATCCCAGAGTGGCCT | |
| | | NALPSDLLENVLC | GCCTATCACAACCACATCCCTTCCCCCCACAAGGCAATAAATCTCATTTCTTTA | |
| | | GVAFGLGVLGIIV | TATC | |
| | | GIVLIIYFRKPCSG | | |
| | | D | | |
| 50 | HLA-DRA | >NP_061984.2 HLA | SEQ ID NO: 100 | |
| | | class II | >NM_019111.4 Homo sapiens major histocompatibility complex, class II, DR alpha | |
| | | histocompatibility | (HLA-DRA), mRNA | |
| | | antigen, DR alpha | TTTTAATGGTCAGACTCTATTACACCCCACATTCTCTTTTCTTTTATTCTTGTCT | |
| | | chain precursor | GTTCTGCCTCACTCCCGAGCTCTACTGACTCCCAACAGAGCGCCCAAGAAGAA | |
| | | [Homo sapiens] | AATGGCCATAAGTGGAGTCCCTGTGCTAGGATTTTTCATCATAGCTGTGCTGA | |
| | | MAISGVPVLGFFII | TGAGCGCTCAGGAATCATGGGCTATCAAAGAAGAACATGTGATCATCCAGGC | |
| | | AVLMSAQESWAI | CGAGTTCTATCTGAATCCTGACCAATCAGGCGAGTTTATGTTTGACTTTGATGG | |
| | | KEEHVIIQAEFYLN | TGATGAGATTTTCCATGTGGATATGGCAAAGAAGGAGACGGTCTGGCGGCTTG | |
| | | PDQSGEFMFDFDG | AAGAATTTGGACGATTTGCCAGCTTTGAGGCTCAAGGTGCATTGGCCAACATA | |
| | | DEIFHVDMAKKET | GCTGTGGACAAAGCCAACCTGGAAATCATGACAAAGCGCTCCAACTATACTC | |
| | | VWRLEEFGRFASF | CGATCACCAATGTACCTCCAGAGGTAACTGTGCTCACAAACAGCCCTGTGGAA | |
| | | EAQGALANIAVD | CTGAGAGAGCCCAACGTCCTCATCTGTTTCATAGACAAGTTCACCCCACCAGT | |
| | | KANLEIMTKRSNY | GGTCAATGTCACGTGGCTTCGAAATGGAAAACCTGTCACCACAGGAGTGTCA | |
| | | TPITNVPPEVTVLT | GAGACAGTCTTCCTGCCCAGGGAAGACCACCTTTTCCGCAAGTTCCACTATCT | |
| | | NSPVELREPNVLIC | CCCCTTCCTGCCCTCAACTGAGGACGTTTACGACTGCAGGGTGGAGCACTGGG | |
| | | FIDKFTPPVVNVT | GCTTGGATGAGCCTCTTCTCAAGCACTGGGAGTTTGATGCTCCAAGCCCTCTC | |
| | | WLRNGKPVTTGV | CCAGAGACTACAGAGAACGTGGTGTGTGCCCTGGGCCTGACTGTGGGTCTGGT | |
| | | SETVFLPREDHLF | GGGCATCATTATTGGGACCATCTTCATCATCAAGGGATTGCGCAAAAGCAATG | |
| | | RKFHYLPFLPSTE | CAGCAGAACGCAGGGGGCCTCTGTAAGGCACATGGAGGTGATGGTGTTTCTT | |
| | | DVYDCRVEHWGL | AGAGAGAAGATCACTGAAGAAACTTCTGCTTTAATGGCTTTACAAAGCTGGCA | |
| | | DEPLLKHWEFDAP | ATATTACAATCCTTGACCTCAGTGAAAGCAGTCATCTTCAGCATTTTCCAGCCC | |
| | | SPLPETTENVVCA | TATAGCCACCCCAAGTGTGGATATGCCTCTTCGATTGCTCCGTACTCTAACATC | |
| | | LGLTVGLVGIIIGTI | TAGCTGGCTTCCCTGTCTATTGCCTTTTCCTGTATCTATTTTCCTCTATTTCCTA | |
| | | FIIKGLRKSNAAER | TCATTTTATTATCACCATGCAATGCCTCTGGAATAAAACATACAGGAGTCTGT | |
| | | RGPL | CTCTGCTATGGAATGCCCCATGGGGCATCTCTTGTGTACTTATTGTTTAAGGTT | |
| | | | TCCTCAAACTGTGATTTTTCTGAACACAATAAACTATTTTGATGATCTTGGGTG | |
| | | | GAAAAAAAAAAAAAAAAAAAAAAAAAAAAAAAAAA | |
| 51 | HLA-DMB | >NP_002109.2 HLA | SEQ ID NO: 101 | |
| | | class II | >NM_002118.4 Homo sapiens major histocompatibility complex, class II, DM beta | |
| | | histocompatibility | (HLA-DMB), mRNA | |
| | | antigen, DM beta | ATTTGTCCCTGCCTACCTAGCCAATCTGTCCCTGTTTGGGACACTGGACTCCCG | |
| | | chain precursor | TGAGCTGGAAGGAACAGATTTAATATCTAGGGGCTGGGTATCCCCACATCACT | |
| | | [Homo sapiens] | CATTTGGGGGGTCAAGGGACCCGGGCAATATAGTATTCTGCTCAGTGTCTGGA | |
| | | MITFLPLLLGLSLG | GATCATCTACCCAGGCTGGGGCTTCTGGGACAGGCGAGGACCCACGGACCCT | |
| | | CTGAGGFVAHVE | GGAAGAGCTGGTCCAGGGGACTGAACTCCCGGCATCTTTACAGAGCAGAGCA | |
| | | STCLLDDAGTPKD | TGATCACATTCCTGCCGCTGCTGCTGGGGCTCAGCCTGGGCTGCACAGGAGCA | |
| | | FTYCISFNKDLLTC | GGTGGCTTCGTGGCCCATGTGGAAAGCACCTGTCTGTTGGATGATGCTGGGAC | |
| | | WDPEENKMAPCE | TCCAAAGGATTTCACATACTGCATCTCCTTCAACAAGGATCTGCTGACCTGCT | |
| | | FGVLNSLANVLSQ | GGGATCCAGAGGAGAATAAGATGGCCCCTTGCGAATTTGGGGTGCTGAATAG | |
| | | HLNQKDTLMQRL | CTTGGCGAATGTCCTCTCACAGCACCTCAACCAAAAAGACACCCTGATGCAGC | |
| | | RNGLQNCATHTQ | GCTTGCGCAATGGGCTTCAGAATTGTGCCACACACACCCAGCCCTTCTGGGGA | |
| | | PFWGSLTNRTRPP | TCACTGACCAACAGGACACGGCCACCATCTGTGCAAGTAGCCAAAACCACTC | |
| | | SVQVAKTTPFNTR | CTTTTAACACGAGGGAGCCTGTGATGCTGGCCTGCTATGTGTGGGGCTTCTAT | |
| | | EPVMLACYVWGF | CCAGCAGAAGTGACTATCACGTGGAGGAAGAACGGGAAGCTTGTCATGCCTC | |
| | | YPAEVTITWRKNG | ACAGCAGTGCGCACAAGACTGCCCAGCCCAATGGAGACTGGACATACCAGAC | |
| | | KLVMPHSSAHKT | CCTCTCCCATTTAGCCTTAACCCCCTCTTACGGGGACACTTACACCTGTGTGGT | |
| | | AQPNGDWTYQTL | AGAGCACACTGGGGCTCCTGAGCCCATCCTTCGGGACTGGACACCTGGGCTGT | |
| | | SHLALTPSYGDTY | CCCCCATGCAGACCCTGAAGGTTTCTGTGTCTGCAGTGACTCTGGGCCTGGGC | |
| | | TCVVEHTGAPEPI | CTCATCATCTTCTCTCTTGGTGTGATCAGCTGGCGGAGAGCTGGCCACTCTAGT | |
| | | LRDWTPGLSPMQ | TACACTCCTCTTCCTGGGTCCAATTATTCAGAAGGATGGCACATTTCCTAGAG | |
| | | TLKVSVSAVTLGL | GCAGAATCCTACAACTTCCACTCCAAGTGAGAAGGAGATTCAAACTCAATGAT | |
| | | GLIIFSLGVISWRR | GCTACCATGCCTCTCCAACATCTTCAACCCCCTGACATTATCTTGGATCCTATG | |
| | | AGHSSYTPLPGSN | GTTTCTCCATCCAATTCTTTGAATTTCCCAGTCTCCCCTATGTAAAACTTAGCA | |
| | | YSEGWHIS | ACTTGGGGGACCTCATTCCTGGGACTATGCTGTAACCAAATTATTGTCCAAGG | |
| | | | CTATATTTCTGGGATGAATATAATCTGAGGAAGGGAGTTAAAGACCCTCCTGG | |
| | | | GGCTCTCAGTGTGCCATAGAGGACAGCAACTGGTGATTGTTTCAGAGAAATAA | |
| | | | ACTTTGGTGGAAATATTGTTAAAAAAAAAAAAAAA | |
| 52 | HLA-DOB | >NP_002111.1 HLA | SEQ ID NO: 102 | SEQ ID NO: |
| | | class II | >NG_009793.3 Homo sapiens transporter 2, ATP binding cassette subfamily B member | 133 |
| | | histocompatibility | (TAP2); and major histocompatibility complex, class II, DO beta (HLA-DOB), | NG_009793.3 |
| | | antigen, DO beta | RefSeqGene on chromosome | |
| | | chain precursor | GAGAGCGGGAGCGCGGGGTACAGGGTCGGGGGTAGCCTTCAGTCCCGGAGAG | |
| | | [Homo sapiens] | CGCCAGACCCAAAGAAGAGGCCACATGGGGATGGGGCCTGAGAGGAGGAAG | |
| | | MGSGWVPWVVA | TGCAAGTTAGGACAAAGAGTTACAGGTGAGGTGGGGGCTCCCAAAGGAAGAC | |
| | | LLVNLTRLDSSMT | AAGAGAACTTTCCTGCCCTTGTCCACACACAAATGGTGGAGCCTTTTTCATGG | |
| | | QGTDSPEDFVIQA | GGTTATCACATGATATAGGAGGTGTGTGGTGTCTTGGGAAACCTATGAAATTT | |
| | | KADCYFTNGTEK | GCCTGCTGGCCTCCTCTCAGCAACTCACTGTTGCGCGACTTAGAACAAGTCAC | |
| | | VQFVVRFIFNLEE | TTAGTCTAGGTCCCAAGCCCCTCTTCTGTAAAGTGAGGATACTGTTACTAAAC | |
| | | YVRFDSDVGMFV | GTGTTTTGTGAGGGTTGAATGCTTTATGCATGGAAGAAACCCTTTAAGTCACT | |
| | | ALTKLGQPDAEQ | CTCAAAATTTTTTAGTAATGGTAACATGTGCTTGCTTTCATTTCTGTTGTGCTG | |
| | | WNSRLDLLERSRQ | GAAAATGGAAAAGGTTGATACTGGCATGCCTCTTCTTTTCCTTCTCCCAAGCC | |
| | | AVDGVCRHNYRL | ATTTCCTTCTAGAGATTGGTTATTAACTGTTTCATTTATTGATGGTTAGATCAT | |
| | | GAPFTVGRKVQPE | TTCTGCATATCTCCTCACCCTCATACTCCCTAAAACCTTTTCCTGGAGCCTCTT | |
| | | VTVYPERTPLLHQ | ACTACAGAATTTTTCATTGCCTTTCTCAACCTCTTTTCTCTTATCAGCCCACAG | |
| | | HNLLHCSVTGFYP | AATTCTTCCAGTCCCTGGGTGGGGACGGAGAAAGGAACGTTCAGATTGAGAT | |
| | | GDIKIKWFLNGQE | GGCCCATGGCACCACCACGCTCGCCTTCAAGTTCCAGCATGGAGTGATTGCAG | |
| | | ERAGVMSTGPIRN | CAGTGGATTCTCGGGCCTCAGCTGGGTCCTACATTAGTGAGTGTATACGCTCC | |
| | | GDWTFQTVVMLE | AGCAGGCAGAATCTGGGGAGCTGGGCTCTCCTTTCCACAGGAGGCCAACTCTG | |
| | | MTPELGHVYTCL | CAACAAAGTGGAAGTGGATATTGATTTAGGACACACTGGGGGATCTATGGGG | |
| | | VDHSSLLSPVSVE | TCATCCCTTCTCTCCCAAAGCTCCATCTTCTTCCAGGTGCCTTACGGGTGAACA | |
| | | WRAQSEYSWRKM | AGGTGATTGAGATTAACCCTTACCTGCTTGGCACCATGTCTGGCTGTGCAGCA | |
| | | LSGIAAFLLGLIFL | GACTGTCAGTACTGGGAGCGCCTGCTGGCCAAGGAATGCAGGTAAGCGAGGC | |
| | | LVGIVIQLRAQKG | CTCTCATCTTCCTTTCTTAGCCTAGTGGTTAATCCCTGGATCTCTCAGATCATT | |
| | | YVRTQMSGNEVS | GCTCCTTACTCTTGTCCTATGTGGTCCATCTTAGTGCTAATAGTATATTTCACA | |
| | | RAVLLPQSC | AAACAGCTTTTTGGTGATAAGACCCTTCTCCCAAATCTCAGCCTGTGCTCCACT | |
| | | | CATAAGCCAGATAGTACGTCAGGTATTTAGCACTGACACATCCACCCTGGCGG | |
| | | | GACAGTATCATTTACTAGGCTGCCTTTGTATGTTTCAGATACTTTAAATTCCAA | |
| | | | ATCTTTCTCTGATCTTTAGATCCTACAAAATAATTCCTTTCCAATGCTTATCTCT | |
| | | | TTAATCATTTCCTGCCCCATCAAGTTGGAAAGAGCTAACCTCTCTTTCCTCACT | |
| | | | CCACCTTGTCCTCACCCAGGCTGTACTATCTGCGAAATGGAGAACGTATTTCA | |
| | | | GTGTCGGCAGCCTCCAAGCTGCTGTCCAACATGATGTGCCAGTACCGGGGCAT | |
| | | | GGGCCTCTCTATGGGCAGTATGATCTGTGGCTGGGATAAGAAGGTGGGTGCTC | |
| | | | TCCATTCTTCATGTTCCCCCACCATGTTCCCTATGGATGACAGATCTGTTTCCC | |
| | | | ATCATATACTCCTACTCCCTCCCTGACAAGATGCATGGCATATAGAGTGCTTG | |
| | | | GTTATAGAACTGTTTCAGTATATCCATGGACTATTTATTGGCCCAGATATGAAT | |
| | | | CATTGCATGTGTTTTGTAAGCTTGTCCCTTTTGTTAAACAGTTGATTTCAAGTT | |
| | | | TGTTTTTCCTTTTTCATTTCTAAAGTTCGATGACTTTACCAAAGTGGTTTCCTAA | |
| | | | TTCCAATGTTCTTGTGGTAATATTAATTCTTTTGTTCACTTTTTATGTCTCATAT | |
| | | | TTGAACCCCCATGTTACCAACATCTTCCTCTCCAATTTCAGCCTGAAATCTTTC | |
| | | | ATCTTATAGGGTCCTGGACTCTACTACGTGGATGAACATGGGACTCGGCTCTC | |
| | | | AGGAAATATGTTCTCCACGGGTAGTGGGAACACTTATGCCTACGGGGTCATGG | |
| | | | ACAGTGGCTATCGGCCTAATCTTAGCCCTGAAGAGGCCTATGACCTTGGCCGC | |
| | | | AGGGCTATTGCTTATGCCACTCACAGAGACAGCTATTCTGGAGGCGTTGTCAA | |
| | | | TAGTAAGAGACCAATGCTCCCACCACCATGCCTGGGAGGAGTCGGCGGGTGG | |
| | | | TGGGGGGGGTGATTTAAGATTGAGAAACCAGCCTGGCCAACATGGCGAAACC | |
| | | | CCGTCTCTACTAAAACTACAAAAATTAGCCGGACGTGGTGACGGGTGCCTGTA | |
| | | | GTCCCAGCTACTTGGGAGGCTGAGGCAGGAGAATCACTTGAACCTGGGAGGT | |
| | | | GGAGCTTGCAGTGAGCCAAGATGGCGCCACTGCATTCCAGTCTGGGCCACAG | |
| | | | AGTGAGACTCCTTCTCAAAAGAAAAAAAAGAAAATGATTGAGAGACTCAAAG | |
| | | | GAGGGAGAGTAGTAGGGAGGAAATTTTCAGAGTCAGAAGAAGGGCATTAAA | |
| | | | GGCCCAGTCATATGGTTTTAAGCTTGCGCATGTGTCTTGTTGCTGCCTTCAACA | |
| | | | TAACATCAGTGACAGGAACTTGCTGAGGTGAAAGGTGACTCCATGTTCTTTTC | |
| | | | TCATTTGTCCACAGTGTACCACATGAAGGAAGATGGTTGGGTGAAAGTAGAA | |
| | | | AGTACAGATGTCAGTGACCTGCTGCACCAGTACCGGGAAGCCAATCAATAAT | |
| | | | GGTGGTGGTGGCAGCTGGGCAGGTCTCCTCTGGGAGGTCTTGGCCGACTCAGG | |
| | | | GACCTAAGCCACGTTAAGTCCAAGGAGAAGAAGAGGCCTAGCCTGAGCCAAA | |
| | | | GAGAGAGTACGGGCTCAGCAGCCAGAGGAGGCCGGTGAAGTGCATCTTCTGC | |
| | | | GTGTTCTCTATTTGAACAAGCATTTCCCCCAGGGAAGTTTCTGGGTGCCCCACT | |
| | | | AAGTAGAATAAAGAAAAACGGTTATAAATACCTCTGTCTTGTGGCTGAATGG | |
| | | | GGTTGGGCCTGTGGTTGTTGGGTGGGGCAGAAAGTAAAGAGACGCTTTTTCTG | |
| | | | GAGAAGGGGCTCAGACCCCTATCTAAGAAATGTGGCCTCACCACATAGTTCTT | |
| | | | CCTGGGGTTTCCTTCCACACTCATCTCTCCAAGACCTCAGGAAGGCTGCTCATT | |
| | | | GCTGTCCATGGACACTTGTTTGCAATTTCACATAGGTTAGGGTCCTTTCCATAG | |
| | | | AGAGGCACCTGGGGACTCCTGTGTGTTCCATTGTTAATGGTTTGAGGAACAGG | |
| | | | GAGTAGGGCACCTAGGATAACTGTTTTTGACTTTATAGAGTAGGATGAAAAAG | |
| | | | CTTCCACTTCACTTTAATATGGTAATCATATAAACACCATACCATTTATCCCAA | |
| | | | ATAACACTTTGGAGATATTGGATATTGAATATAAAGACAGACATTAAGGGTCT | |
| | | | AATTTCATGATGTGTCATGCTGAATTGCAAGATGGCAGGACTATAATTTTAGA | |
| | | | GGAAGAAGAGATCAGGAGGACTCCCCTAAGTGAGGAGTGTGGGGAAAATGTA | |
| | | | AAAGATCCAGGTTAGAAGAAAGAGACACACATCATGAGATTTTCGGAATCAT | |
| | | | GCTGGAACTATGGACCATGTCACTTTCCAGAAAATAAAGGAAACAAATGCTT | |
| | | | GAAAGTAGGAGCATGAGCTTGAGCATGGAGCTTTTTTCATTAGAGAGAGATTC | |
| | | | TTAAAATGCCAGAATGAATAGAGTTGTAAAACTTTAGCGAGCCCCTACTTAAA | |
| | | | ATCCCTCCTCCCCACCACCACTATTTTAAACTTTAAGTGACTCCTTGGTAGTCA | |
| | | | CACAGGTAACATTTCAAAATGGTGATCTGGAATCCAAGCGACCCTTCCTTTGG | |
| | | | GGAATTTGTTTGTTTGTTTGTTTTTAGTTTGGGAGGTGGTTTCAATCTGAAGAG | |
| | | | TCCTTTCTGGAATAAACGAATCTTTCTGTTGCATAGGAAGGCTCTGGGTCAGG | |
| | | | AAGGATATTTAAAAACCTAATTACTGTTCTAAACAGTGTTAAAATAGAACAAG | |
| | | | AACCAAAGCTCAGTACGGGGCATTTCCCTCATAGGCTGAAGGTGCGCCCAAC | |
| | | | ATAATTTGGAGTACAGACTCAGAGGCACCTGAACACGCGCCAGCTCAAGGTG | |
| | | | CTCCGGCTGAGAAGGACGGATGAAGATGAACGCTCAGGGCCTACTAAATTCA | |
| | | | AAGTCTGTACGTGAAAATCCCCTTTGGCCTGGTGAGATTGGTTGGAACCTTCT | |
| | | | ATTTAGGAGAGCCCGGCTCGCTCGCCTAAAACTGGAGCTTGCATGGAAGAGG | |
| | | | GCACTTTTTTTTTTTTTAGACGAAGTCTCACTCTTGTCGCCCAGGCTGGAGTGC | |
| | | | AATGACCCGATCTCGGCTCACTGAAACCTCTGCCTCCTGAGTTCAAGCGATTC | |
| | | | CCCTGCCTCAGCCTCCCGAGTAGCTGGGATTACAGGCGTCCGCCACCACGCCC | |
| | | | TGGCTAATTTTTGTATTTTTAGTAGAGACAGGGTTTCACCATGTTGGCCAGGCT | |
| | | | GGTCTCGAACCCCTGACCTCAGGCGATCCGCCCGCCTCGGCCTCCCAAAGTGC | |
| | | | TGTGATTACAGGCGTGAGCCACCGCGCCGGACCAGAAAGAAGGCACTTCTTA | |
| | | | ATAGTAGGCTCAGAGCTTGAAGTAGTAACTTTGAGAAAATTCAGTGATTCTCC | |
| | | | AATTACAAAGCATTCTCCAATTACAAAGCAAGGACAACAGATAAAGTTGCCC | |
| | | | TTGAGACAACTGTATTTTACTTAATGATAAAGAAACATTTTTGCAGTTTTATAT | |
| | | | CCCAGAGTAACCGCCACTAAAGGCGAGTGAGACTCATTGCAGGCCTGTACAG | |
| | | | TGCGAACCAGAGTTCGGGCTCCAGTTCCGCTGTCTGCGGGTCTCGCGCGCCCC | |
| | | | CTCCCGGCGGCCCAGCCCAGAATGAAGGCCTTGGCTGGGGAAGCGAAAGCGA | |
| | | | AAGCTGCCCGAGCCCTGACGCCCGCCCTGGCCGAGCGTAGCTGGCGGACCAG | |
| | | | AGCCGGTAGCGAGGTTGGGAGAGACGGAGCGGACCTCAGCGCTGAAGCAGA | |
| | | | AGTCCCCGGAGCTGCGGTCTCCCCGCCGCGGCTGGTGAGTTGGTGCGGAGGG | |
| | | | GAACCTGGAGCGCCAACAGGGACGCAGCCCAAGTGACTACCCACTCCACGCT | |
| | | | CCTGCTTCCCAGTCCCTCTGCACCCGGCGATAGGAGGGAGCGGAGCCCGGACC | |
| | | | ACTTAGCTCGCCGCGGCAGGCGGGGGTGGGGGTGGGGGTCCGGGGATTTTTTT | |
| | | | TTTTTTTTTTTAAGCACGAGGCTCCTGATGGTCATGCTTCCAGCTCCCCAGAAG | |
| | | | GCCGAAAGCTGTCTGTCGTAGGAGGGGTGTACGGATGAGCACCGGTTACTCA | |
| | | | GGAGAGCTCTCAGGGTTGAATAGGATAAAATGAGAAGCCGATGGACGGGTTA | |
| | | | GGCGGAGCCGGGCGGGTAGGAGGGCAGGGACAAGGATTGGGACTCCACCCCC | |
| | | | ATGATTTCTCATCTCGTATCCGTTGACAGAGCCATGCGGCTCCCTGACCTGAG | |
| | | | ACCCTGGACCTCCCTGCTGCTGGTGGACGCGGCTTTACTGTGGCTGCTTCAGG | |
| | | | GCCCTCTGGGGACTTTGCTTCCTCAAGGGCTGCCAGGACTATGGCTGGAGGGG | |
| | | | ACCCTGCGGCTGGGAGGGCTGTGGGGGCTGCTAAAGCTAAGAGGGCTGCTGG | |
| | | | GATTTGTGGGGACACTGCTGCTCCCGCTCTGTCTGGCCACCCCCCTGACTGTCT | |
| | | | CCCTGAGAGCCCTGGTCGCGGGGGCCTCACGTGCTCCCCCAGCCAGAGTCGCT | |
| | | | TCAGCCCCTTGGAGCTGGCTGCTGGTGGGGTACGGGGCTGCGGGGCTCAGCTG | |
| | | | GTCACTGTGGGCTGTTCTGAGCCCTCCTGGAGCCCAGGAGAAGGAGCAGGAC | |
| | | | CAGGTGAACAACAAAGTCTTGATGTGGAGGCTGCTGAAGCTCTCCAGGCCGG | |
| | | | ACCTGCCTCTCCTCGTTGCCGCCTTCTTCTTCCTTGTCCTTGCTGTTTTGGGTGA | |
| | | | GTCAGGAGAGGACGTTGTGAGTTGGAGGTGGTAAAAGGGCCTGGGCACCAGC | |
| | | | ACATTCTTGTGTTATTTTTCATGCCTCTTTCAGGTGAGACATTAATCCCTCACT | |
| | | | ATTCTGGTCGTGTGATTGACATCCTGGGAGGTGATTTTGACCCCCATGCCTTTG | |
| | | | CCAGTGCCATCTTCTTCATGTGCCTCTTCTCCTTTGGCAGGTAGGTGGTGGGCA | |
| | | | GCTGGGTCCATTTGCTAGCCCCAAATCTTTATAGGGGTCTTCACTTCCCTAACT | |
| | | | CCATTTCTAGGCCCTTTCAGGCGCAAAACACAAAAATACTTAAACTAAAATAT | |
| | | | GGTGAATGTAGTCACCATTCTGTTTCATCTATCCATTCATTTCTTCCTTCGTTCA | |
| | | | TATTCATCCAATATCTTCAAAGTTTATCTGATCTTATTATAGGAACAAGTTATG | |
| | | | AGTGAAGGTAGTACAAAAGGAATTTAAGTCTCAGATGGAATGTCTCTCAGTTG | |
| | | | TCTCTCAACATTCCTAGGTCCATGAAATTCCATTTCTTTCTGCCTCCTACCTCCT | |
| | | | ACCCCTAAGTCTGTCTCCAAAGTATCTCTCCAGGGTCACTCCCTCAGGATGGG | |
| | | | TATGCTTCTCCCTTTCACTCTTCTTTCCCAGCTCATCTTGCCAGTCCCTGAAGAT | |
| | | | CTTACTCTGAGGCTTATCACCTTTCTTTCCAGAATCATTACTCTTTTCCCTTCAC | |
| | | | TTGCTTTCCTTTCTCTTTCTAGACATACTCAAACAAACAAACTGTTTGATAAGG | |
| | | | CTGGGACTGGGATGAGGTGAGCGAGGCACCTGGGGTGCAAAGTTTAAGGAGG | |
| | | | TGTGCACTCACCTTACCCAAATCCCAGCCGGCCTGATTGTCTCTATTTTTATGG | |
| | | | TCATACTTAATTTAGAGTACCCTCGAAAACCATTTCATTGTGCCTTCATTCCAT | |
| | | | CCTGGCTGCTTTCTGCTGAAACTACAGTCGTGCACTGCATAACGAAGTTTAGG | |
| | | | TCAATGATGGGCCACATATAAGATGGTGGACCCACAAGATTATAATACCATAT | |
| | | | TTTTACTGTACCTTTTCTATGTTTAGATACACAAATACTTACTTCTGTGTTACA | |
| | | | GTCGCCCACAGTGTTAGGTGCAGTTATATGTTGTACAGATTTGTAGCCTAGGA | |
| | | | GCAATAGGCTAAACTACATGGCCTAGGTGTGCAGTAGGCTATGACATCTAGGT | |
| | | | TTGTGTAAGTACACTCTATGATGTTCATACAACGATGAAACCATCTAACGACA | |
| | | | CATTTCTCAGAACACATCCCTGTCATTAAAGTACAAACCCTCATTATATCATGT | |
| | | | CTGACTCTCCCAAACGCCTCTTCAATATGGTAACTAAATTTGTATTAGAAACA | |
| | | | TATATTTTGTAAAATACATGCTTTTATGCTTTATATTTTTTCCTCTAAGTGTTAC | |
| | | | TGTAGCATGTAGTTGGTCTAAGAGGGATTTTCCAACTCGAAGGATGAAAGATG | |
| | | | GGAATCACATGACTCTGGGGCTCCAGAGAATTGTGGGGGCAGGGAATTTATT | |
| | | | ATTGCAGTTCCCATGATGAAGTATCTATGATGACAGAGAAGGGCTTTGGGTAT | |
| | | | GGGGCAGGAAGGAGACCAAGGCGGAGGAGACGCACAGAGGGACAAGCCTGA | |
| | | | GGGACGCTGGGACAGAAGCAAGCACTGGGATACTTGTTTTCACAATATCTTTT | |
| | | | CCCTTCTATTGTAGTTTTCTATTGTGTCTAGTACAGAGTGCACTCCATAAATAC | |
| | | | TTGTAAATTTGTACATGTTATGATTTTGTTCTCACATCTAGCTCACCATGTCTC | |
| | | | CTCTTTCTTCTTCCTCTGTGTATTCCTTACCTCTTCTCTCTCTGTGTGTCTGTCTC | |
| | | | TCATTTCTTTCTCTTTTGCCCCTCCTGGCATGCTTTCCCCTGACTTTGCGCTTCT | |
| | | | CTGCACTCCTGGCTTGCTCCTCTGTTTCACCCGCTGGCTTGCTCCTTCTCTGCAT | |
| | | | CTCCCTCCCCTCTTATTCTCCTACCCCACAGCTCACTGTCTGCAGGCTGCCGAG | |
| | | | GAGGCTGCTTCACCTACACCATGTCTCGAATCAACTTGCGGATCCGGGAGCAG | |
| | | | CTTTTCTCCTCCCTGCTGCGCCAGGACCTCGGTTTCTTCCAGGAGACTAAGACA | |
| | | | GGTGGGGCCTGGAGTCCAGGTCTGAGATTCCCATGGACATCCCTTGCCCCTCA | |
| | | | GTGACCTTCCACCCACAGCCTCTCCTCCTGCCTTCACCCGTATGCCAGGACTTG | |
| | | | GGGATGCTTTTCTCTTGTTTGGGACAGGGTGGAGAAGCAGCCTCCACTGTCCC | |
| | | | TCTGCAAGTGAAGGAGGATGTTCAGAGGAGGGGGCTGTGTCAGAGGGAACGG | |
| | | | TCAGGAGGGAGTTTCTGGGGGCCCTGCAGTACACATGGTTTCCTTTTTCCTCAC | |
| | | | CTGCTCTGTCCTTCTTAGGGGAGCTGAACTCACGGCTGAGCTCGGATACCACC | |
| | | | CTGATGAGTAACTGGCTTCCTTTAAATGCCAATGTGCTCTTGCGAAGCCTGGT | |
| | | | GAAAGTGGTGGGGCTGTATGGCTTCATGCTCAGCATATCGCCTCGACTCACCC | |
| | | | TCCTTTCTCTGCTGCACATGCCCTTCACAATAGCAGCGGAGAAGGTGTACAAC | |
| | | | ACCCGCCATCAGGTGAGCGTGCATGTAAGGGAACCCCAAAGGGAGAATAAAA | |
| | | | CTGACAGGTGAGGAGGCTTCCACATTTGTGGCTAGAGGATCCCCTAGAGAGA | |
| | | | GATGTTCTCTTCTCAGCCATTAGGGGAGAAGGTATATTGTAGTATATACTACA | |
| | | | TTTTGTTTGTCCAGCCATCCAACAATGGATATTTGACTTCAGAAGATTCATGAT | |
| | | | TCTCCAGAACTGTAAACAAAAATGTAAAGTGTATGTGAAGGTATGGGGGAGG | |
| | | | GAATAGGAAGGGGAGATGATAGGCGATGATAACTTTTCATTAGCTTCTCAAA | |
| | | | GGAGTCTGTACATCCCCCGCCCCCACTGCGAAGATTAAAAATGGTTTCTTAGA | |
| | | | GGCTTTTAGGCAGGGAGATTTTCCCTTTAAAATCAGCAGAAGAAGTCTGGATG | |
| | | | CAGCATAGGGAAAGGAGGCGTCATCAGGAAGTCCTAAGTCTGAATGTCAGCT | |
| | | | CCACCTTCTCTTTTTCTCTTATATTGTGGTAAAACATACATAACATAAAATTTA | |
| | | | CCATTTCAACCATTTGAAGTGTACAGTTCAGTGACATTTAGGAAACCCACATT | |
| | | | GTTATTGGGTAACCATCATCACCATCCATCTCCAGAACTTTTTTCATCTTCCTA | |
| | | | AAATGAAACTCTGTACCCACTAAATAGTAACTGCCTACTACCCCCAACCCCTG | |
| | | | GCCGCTGGCAACCTCCATTGTACCTTCTGTCTCCATGAATTGTGATGACTCCCG | |
| | | | GTGCGGTACGTAAGTGGAACCATACAGTATTTGTCTTTTTGTGACTGGCATATT | |
| | | | TTACTTAGCGTAATGTCTTCAGGCCTCATCCATATTGTAGCATGTGTTAGAATT | |
| | | | TCCTCCCTTTTAAGGCTGAATAATATTCTGTTGTGTGCATATATCACATTTTGA | |
| | | | TTATCCATTCATCTGTCAATGGACATTTGCGTTGTTTCCACCTTTTGGCTGTTGT | |
| | | | GAATTATGCTGCTGTGGACATGAGTGTACACCTGTTTGAAACCCTGCTTTTGGT | |
| | | | TCTTTTGGGTATATACTTAGAAGTGGAGCTGCTGGATCATATGGCAATTCTATT | |
| | | | TAACAATTTTTGAGGAACCATGTATTAGTCCATTTTTACGCTGCTGATAAAGA | |
| | | | CATACCCAAGATTGGGCAATTTACAAAAGAAAGAGGTTTATTGGACTTACAGT | |
| | | | TCCATGTGGCTGGGAAGACCTCAAATCATGGCGGAAGGTGAAAGACACATTT | |
| | | | CACATGGCAGCAGACAAGAGAAAAGACAGCTTGTGCAGGGGAATTCCCCTTT | |
| | | | TTAAAACCATTATATCTCGTGAGACTTATTCAATATCACAAGAACAGCATGGG | |
| | | | AAAGACTTGCCCTCATGATTCAATTACCTCCTACCCAGTCCCTCCCACAACAC | |
| | | | ATGGGAATTCAAGATCAGATATGGGTAGGGACACAGCCAGATCGTATCAAAC | |
| | | | CACCATACAGTTTTCCATAGCGGCAGCACCATTTTAAATTCCCACCAGCAGTG | |
| | | | CATAAGGTTTCCAATTTCTCCACATCCTCATCAACACCACTTTCTGTTGTCTTTT | |
| | | | TTTTTAATAGCCATTCTAATGGTGATTAGGTGATTAGGATTATCTCATTGTGGT | |
| | | | TTTGATTTGCATTTCCCTAATGATTAGTAAATATTGAGCATCTTTTCGTGTGAT | |
| | | | TTTGGCCACTTATGTTTCTTTCTTGAAAGAATGTCTGCAAGTTCTTTGCCCATTT | |
| | | | TCTGATTTTTTTTTAAGTTGTGGGAGTTCACTATATGTTTTGCCTATTAATTTCC | |
| | | | TATCAGATATATGATTCACAAATATTTTCTTGTATTTCATGGTTGCTTTTTCACT | |
| | | | CTGTTGCTAGAGTTCTTTGATGCACAAACGTTTTAAATTCTGATGAAGTCTGAT | |
| | | | TTATCTATTTTTTGTTGCCTGTGCGTTTGGTGTTATATCCAAGAAATCACTGCC | |
| | | | AAATCTAGTGGCATGAGGCTTTTCTTCTACATTTTCCTAGGAGTTTTATAGTGT | |
| | | | TAGCTCTTATGTTTAGGCCTCTGATCCATTTGGAATTACATCTCCACCTTTCTT | |
| | | | AACTATCTGTGGCTCCTTGGGAAAACTACCCTTCTTTCCTGATTCAGACACTGG | |
| | | | GGATGGGAAAATTACCTCAAATGAAGGTTAAAAAAATTGCATGTATCTCCTAT | |
| | | | ACTACCTAACACTGAGAGCTCAATAATATTTTGTTCCCTTGCTCCTTCACTCTT | |
| | | | ATTCCTTCTGGAAAGAAGAGTAAGGAAGAGGGAGAGAAACAGTTTGGTATTT | |
| | | | TTAGGTAGACTAGGGAGCATCTCACTGGCTGGAGTAAGATGTGGGGGCCTGCT | |
| | | | GTCTTTGCACATCAGCCCTGGTGTTTGCTGGCCCTCTTTTCCAGGAAGTGCTTC | |
| | | | GGGAGATCCAGGATGCAGTGGCCAGGGCGGGGCAGGTGGTGCGGGAAGCCGT | |
| | | | TGGAGGGCTGCAGACCGTTCGCAGTTTTGGGGCCGAGGAGCATGAAGTCTGTC | |
| | | | GCTATAAAGAGGCCCTTGAACAATGTCGGCAGCTGTATTGGCGGAGAGACCT | |
| | | | GGAACGCGCCTTGTACCTGCTCGTAAGGAGGGTAAGATACCAGAGTGGTTGT | |
| | | | GAAAGGAGCCCAGGAAAGGGGGAGGGCAAGGGAAGAGGAAACTACAGCTGG | |
| | | | TTCTAGAGGCCTTTGCAGCTCAGTCTCATAGAGGCAGAGAGGGGGAAAGAAT | |
| | | | GGGAAGATTCCCAGCCTCATCTCTTTCTTCTCCTCTTCCAGGTGCTGCACTTGG | |
| | | | GGGTGCAGATGCTGATGCTGAGCTGTGGGCTGCAGCAGATGCAGGATGGGGA | |
| | | | GCTCACCCAGGGCAGCCTGCTTTCCTTTATGATCTACCAGGAGAGCGTGGGGA | |
| | | | GCTATGTGCAGGTGAGCGAGAAGCCAAGCCTGCTCTCCTTTTTTCCCTCTCTTT | |
| | | | TTCTTTGTGGACTCCTGGGCCTTGGGCTTTATTTGTTCTTTTTAACAATACAAT | |
| | | | ACAAAACCAAAACCCGCAAGTAATTTTGCTATGGAGAATTTTAAACATATGCC | |
| | | | AAAAATGAGACAAAATAATATTACAAACTCACATGTATACATCCTGTGCTTTA | |
| | | | ACAATGATCAACTCATGCCCAATCTTGTTGGATCTGTATCCCCAGCCACTTCCC | |
| | | | CCCACCCATATTATTCTGAAGCAAATCCAAGATATTGTATACTTTCATCTGTAA | |
| | | | ATATTTCAGTATGTTTCTTAAAAATACAAACATCTTTAAAAGTGTATAACAAC | |
| | | | AAAGCCATTATCACACCAAAAAATTAACAGTAGTTCTTAAAATTTATCAAATA | |
| | | | GTCAATTGTCAAATTTCCACTTGTGGTATCCATGTAGTATATGTGTATGAGTGT | |
| | | | GTTTATTATACTTTGCTTAAATCAGGATCCAGAAATGGTCCACATATTGTGACT | |
| | | | GGTTGATACATCTTTTAAGTCTGTCTGTCTATCTATCTATCCATCCATCTATCC | |
| | | | ATCCATCCACCCATCAATCCATGTATCTGTCTAAAAGTTTCCCTTGCACAGTTT | |
| | | | ATTTGTTGAAGAAATAGGTTGTTTGTCCTGTGGAGTTTCTTAGGGTCTGGATTT | |
| | | | TGTTGACTGAATCCCTGTGGTATTATATGCTCTTCTGCCTCTGTACTTCCTGTCT | |
| | | | ATTGATAGATAAACCTAGAAGCTTGTGAGATTGAGGGGTTTTTTTTGGTCTTTT | |
| | | | TCCAGCAACAGTACTTTTTAGGTGGTGATGCATTCTTCCTCCAAGAGGCACAC | |
| | | | AATGTCTGGTTCTCTATTTGTGGCAACATCAGCCACTGATGAGCAGTACCTAC | |
| | | | ATCCATGACAGAATTAGGGCTGCAAAAGGGAGATACTCTATCATTCTTCATGT | |
| | | | ATTAGCTGAAGTAGTCTATGAAGGGAGACTTCCCCTCATCTACCATTTCATTA | |
| | | | CCCGGTGGTACAGTTTGATGAGGAAAGGCAGAGTGAGCATTTAGATCTCTTCC | |
| | | | TACATTTACCAGTTCTCAAAAACAGCTACTTCATCCAGGGCTTTATTTAAACAT | |
| | | | TTTCCTAGACACTTGATAAACATCTTTTTTGTGTAGAGAACTGCGCTGGGCACT | |
| | | | CTGACGGCTACAAAGGTGAGTTGGGCACAGTGCCTGCATTTAAGGAGCTCCCC | |
| | | | GTCTAATCAAGCAAGACAGAACTGGGCACAAGTAATAGGAAGCAGTAACTGA | |
| | | | AAAGATCTGGGGCTAGAGGCAATGCTGTATGGTAGGAGAAGGGACTGTATAT | |
| | | | CCTTTATATTGCAAATTGGAACACTGGGGTATTGGTGCCACTTTTAAATTCCGT | |
| | | | CCAAATTGTACATTTAAAAGTGGAGAATCTCTTTTGAGTATGGAGGAGGAGCA | |
| | | | GTGCAGTTGTGAGTGGAGTGTGTGAGGAGTTGGGAGGGTGGTTTCTGGTAGA | |
| | | | AGTGTGTTTAATTAGCCGGCTCTCCCATTCCTGTTTTCCAGACCCTGGTATACA | |
| | | | TATATGGGGATATGCTCAGCAACGTGGGAGCTGCAGAGAAGGTTTTCTCCTAC | |
| | | | ATGGACCGACAGCCAAATCTGCCTTCACCTGGCACGCTTGCCCCCACCACTCT | |
| | | | GCAGGGGGTTGTGAAATTCCAAGACGTCTCCTTTGCATATCCCAATCGCCCTG | |
| | | | ACAGGCCTGTGCTCAAGGTGCCTGAAAGAGGGAGGAAACCTGGACCCTTGCT | |
| | | | CTCTGCTGCTAATGCATAATTGGACATCACAGCCTATAGTTCATTTGCCTCTGA | |
| | | | GAACCTGGTCTTGCCTCTGCTAAGAAGAGAAATGGAGGGATTTTGAGGGAGA | |
| | | | AGGGGCAGGCCCTTAACTCTTTTTCTGGTTTTCTAGGGGCTGACGTTTACCCTA | |
| | | | CGTCCTGGTGAGGTGACGGCGCTGGTGGGACCCAATGGGTCTGGGAAGAGCA | |
| | | | CAGTGGCTGCCCTGCTGCAGAATCTGTACCAGCCCACAGGGGGACAGGTGCT | |
| | | | GCTGGATGAAAAGCCCATCTCACAGTATGAACACTGCTACCTGCACAGCCAG | |
| | | | GTGGGTGAGGAGGGAGAAGACAGGGGACAGGAGAGGGGAGCATGTACAGAG | |
| | | | AGAGGATGGGAGATCCACGGGAAGGCGCACCAGGTGTTCATTCTGAGGGAGG | |
| | | | TAGGTGGGGAGGACAAAAGGGCCCCTGCCTTGGGGGTTTACACATAGTCCTCT | |
| | | | GCCCCTGTCCCTGCTGCACAGGTGGTTTCAGTTGGGCAGGAGCCTGTGCTGTT | |
| | | | CTCCGGTTCTGTGAGGAACAACATTGCTTATGGGCTGCAGAGCTGCGAAGATG | |
| | | | ATAAGGTGATGGCGGCTGCCCAGGCTGCCCACGCAGATGACTTCATCCAGGA | |
| | | | AATGGAGCATGGAATATACACAGGTATCTTCTACAAATTGTAAGCTTGCTCCT | |
| | | | TCAGTAAAAAAGAGAAAATCAGACTTACTCTTAGTGGTGAAGGTCGTGTCCCT | |
| | | | GTAGCTTGATGTTTGCTGTTCCTCTGCCCTTTCCTCCATTCCTACGTCTCCTTCC | |
| | | | CCACACACTGAATTCTTCAGCCTCCCTCTTGATCAAGAGTCTTTGTTTGCAGAG | |
| | | | AGCAATGCAGCAGTGGTGCTCCCTCCGTGGGCAGCCCCGTCAGGTCCCCACCC | |
| | | | CATGGCCCTCCTCCCACTGGGCCCTCCCCGCACTGGGCCCTCCCACCTCCCGA | |
| | | | GGTCCTACTGGAAGTACCTGCTGTGCACTTGTCCCTCCTTGTGTGTTGTCTGTG | |
| | | | TCACTTGTATCTGAGGAAGGGAATTTCTCTGATTTCCTCAGATGTAGGGGAGA | |
| | | | AGGGAAGCCAGCTGGCTGCGGGACAGAAACAACGTCTGGCCATTGCCCGGGC | |
| | | | CCTTGTACGAGACCCGCGGGTCCTCATCCTGGATGAGGCTACTAGTGCCCTAG | |
| | | | ATGTGCAGTGCGAGCAGGCCGTGAGTACCGTGAGAGGGCAGGGGACAGTGGG | |
| | | | GCCTGGGAGGGGCATGCTGGGAGGATCAGACTGTGCAGAATTGGGCAGAGGG | |
| | | | AGGACGAAGGACCTACTAGTGGAAACAGTCTGTGCCTTCTTGGGGTTGGGGA | |
| | | | ATGGAATCCGGTGGTGTGAGGGCAGCCCCAGTTCCCTCCTGGGCTTCCATTCC | |
| | | | TCCAGCTGTGGCAGTACAGCCGGGAGAGAAGGGCAGTCCAGGCCTTTATCTA | |
| | | | CTGCCCTTTCCTACCTTCTTTTATTTCACACCTTCTTTACCCTAAATCATAAGAG | |
| | | | ATGGTGCCCAGGTGGATGTGGTGTCCATCTCATTCCTGTCTTTCTGAGGCACTG | |
| | | | TGATCACCCCTTCAGCTGCAGGACTGGAATTCCCGTGGGGATCGCACAGTGCT | |
| | | | GGTGATTGCTCACAGGCTGCAGGCAGTTCAGCGCGCCCACCAGATCCTGGTGC | |
| | | | TCCAGGAGGGCAAGCTGCAGAAGCTTGCCCAGCTCCAGGAGGGACAGGACCT | |
| | | | CTATTCCCGCCTGGTTCAGCAGCGGCTGATGGACTGAGGCCCCAGGGATACTG | |
| | | | GGCCCTCTTCTCAGGGGCGTCTCCAGGACCCAGAGCTGTTCCTGCTTTGAGTTT | |
| | | | CCCTAGAGCTGTGCGGCCAGATAGCTGTTCCTGAGTTGCAGGCACGATGGAGA | |
| | | | TTTGGACACTGTGTGCTTTTGGTGGGGTAGAGAGGTGGGGTGGGGTGGGGTGG | |
| | | | GGGCTGTCTGTGTCCAGGAAACTTAATTCCCTGGTGACTAGAGCTTTGCCTGG | |
| | | | TGATGAGGAGTATTTTGTGGCATAATACATATATTTTAAAATATTTTCCTTCTT | |
| | | | ACATGAACTGTATACATTCATATAGAAAATTTAGACAATATAAAAAAGTACA | |
| | | | AAGAAGAAAAGTAAAAGTACCCATTGTTTCACTTCCTGGAGATAACCATAGTT | |
| | | | GCTATTTTGCTGCCTGTCCCATCAGTCGTTTATCTGTTGTTTGAGATAGAAATT | |
| | | | AACCAAAAATGACATAAATATTCATGAGATTGCCTTCCTATATCCTTCCTTGTT | |
| | | | CCTACCAGTGTCTGCTATTTTGAAGAAGCTAGGGTCTGGAGGGACAGAGAAC | |
| | | | AGTTCCCTGATTAACAGTATTAATAGCGACATTGGTAACAGCTACCATTTATA | |
| | | | GAGTTTTAATGGGAGTAGGAGCTATGCTAAGTGTTTTTCATGTATTATCGTTTT | |
| | | | TAATCATTATCCCCAACCCTATGAGGTTGGTTATTATCCCCATTTTACAGATGA | |
| | | | GGAAACTGAAGCTCAAAGAGGCTCAATGACTTTCCCAAGGTGGTCGTAGTGG | |
| | | | TGGAGTTGGAGTTTGAACACAGGCCTGACCCTAGAGTCCACACCCTGACCCAA | |
| | | | TCAATTATATTGCATCTTGGGTCCATAAACCCTAATCCATAATCCCATCAAGA | |
| | | | AAAGCTCTGCTGCTCTTAGCTCTAAATAATTCAGAATCTATTCTCTTCTCTCCA | |
| | | | GTCCCGTTGTTATAGTCTTCACTCATAGACTTAAGATGATCCCATCACCAGAG | |
| | | | AGGTTTCTCTACCATTAGCTTCCCTCTTCCGGCCATTCTTCACAAAGTCATTTTT | |
| | | | CTAAATTCTGTGTCACATACGATGATGGCATTTCTGGAAATTCCTTCAGGTGCT | |
| | | | CTCAAGCCCTGCTGCAGAGATCCTTTTCAGAGCACACACTGTTCCAGCCCATC | |
| | | | TGTCTCACCCTCTCCTGTTGTATCCAGCTCCACGACAAACTTCTGCCTTCCCCA | |
| | | | ACACCTTTGTGCCTTTGCATATGGTGTTTTCTTGCCCATTTTCTGCTCGACTCGC | |
| | | | CCCTGATTTTCAAGTTCAAGACTTAACTCAGGGTTCAGGTCTTCCAGGAGGCC | |
| | | | TTACTTATGTCGTCAGTCTGGGGAACTCTCCATGTGCTTCTATCACTGTGCGGT | |
| | | | TACCTCTTTCACAGCCCTTTTAAAGTTCTATCTTCCCTTTCCCACCTTTTTTGAC | |
| | | | CTTCCACTAGACCATGAGCACCTGGGCGGAAAGCCATATATCTTATTAAGCTT | |
| | | | TATATCTGCTACCTGGCCGAGGGCCTAATTCATAGTGGAGAATAAATAGTCAA | |
| | | | TTGAATAAATGAATAAATATCTCCACCATCGTACTAATCTTAATCCTCCCTGCC | |
| | | | CACTCCCACCACTGAAAATGCAACATTGTACACATCACTGGTTGTTGGGAGGG | |
| | | | ACTTACCTTGGAAAGTTGCTATTCTAGGAAAGAGAAACCTTCATATTCCTGGA | |
| | | | AACAGCAGGTAGTTTCCAGTGCTGGCAATGAATTCCCCAGAACTGCTGTTTTG | |
| | | | GATTTTTTCTTGCCTGGCAGCTGTTGGGAGCAGGGTGCAGTGAGGATGGGGTG | |
| | | | AGAGTGGGCAGTTTCTTGTGCAGATTTGCCTTTCTTTCATCCTGGGGCTGACTT | |
| | | | GCAGCTCCACACCCATCCATCTCTCAAATTTCACAGAGGGTAAAATAGGCATT | |
| | | | TGGAGAGAAAGAACTCTGGCCTGATTCCTTTCTCTCCCACAAATGTCCTTTATT | |
| | | | CATAAAACAGGAATAATAATTCCTGTATCTCCCAACTACATGGAAGCTGCAGC | |
| | | | CCTCACAGAAGAAGATGATCTGAGAAATTCTTTGATTTCCTCAGTACAGTTAT | |
| | | | ACCCATGCATCATAATACTTTAAGCCTGGAAGGCATCTTAAAAATAATGCAAC | |
| | | | AGTCAAACCTAATTTTACAGAGAAACTGACATGAAATCACGCAGCTAATCATG | |
| | | | ATAAAGCTGGGTGGAAAACTTATCTTGATGGGCAGTACAGGAAGATGCAGTA | |
| | | | GACCTTAAGATGTCCTGAAAGTTTCTTATCTCAGGGGAAACTCCCAGGTAGGC | |
| | | | TTTATGTCAGGGACACAGAAAAATGCTCCCTGAAAGTCAAAATATTCGGGCTA | |
| | | | GACAGACAAATTCCTGTAAGTGTGGTTTGTCTGGGAACCACAGATGTCACTAA | |
| | | | TCCTGGTTTGCTCCAGAGTTCTTTTTGTTCACTCCTACCCCCCATCACCATTTGA | |
| | | | TTGATCTCCTTACCCTGTAATTTCCCCTTCTTGTCGCTTACCTGCAGTATCTTTC | |
| | | | CCACCCAGGCATGCCTTATTCTTTCTAAAGGAAAGTATGAATGGAGAGGGGA | |
| | | | AAGCTTGGGAAACTGATAGATTTCCTTGGATGCCAAAACACCTCCATAGCCTG | |
| | | | TCTGCCCGGCCCTATGTGGAAACAGCATTGAGTTTCAAGTCCTTTATGCCTCCA | |
| | | | CCCAGGGATAGCCACTTGTAATCCACATGGCAATTGTGAAACAAGCAGGAAA | |
| | | | TGCGTAATTGTCAGAATTTTGTGGGGAAAGGACTAGGGAATAAGGAAAACAA | |
| | | | AGATCTTCCTTGTGTTTTAGAGCTGTCAGCTAGAGGAGCACCTGCTTGAGTCT | |
| | | | GATGCCATCTAATGGTCCCAGAAGAAACTGGGTTTTGAACCTAGAGTTCCATG | |
| | | | GACTCTTAGGAATTAGACTACTACTACTACTAAGCATTCACTGGTGCTTACTAT | |
| | | | GTGCTATTGCTGTGCCAAGTATCTGAAACCTGTCTTCTTACCTTATTTTTCAAG | |
| | | | ATAATTCTATGTGGCAGGTATTACTATCTCAATTCTAAGAGTGAGAAAATGGA | |
| | | | GTTTTAGAAACATTTACTAACTTGCCTGGGTCACATAGCTAAGGAAGAGGTGG | |
| | | | ACTTGCCCAGCTTTGCATAAAACTCCTCAAAAGAGTTGCCTATACTCCCTGAC | |
| | | | TCCACTTATCTTCCTACTATCCTCTTTTTAAAATATATTATTTATTTATTTAAAT | |
| | | | AAGCAATATATGAATGTGGTTTGAAATTCAAAAGACACAAAGAAGTATACAG | |
| | | | AGGAAAGCCTCACTCTCAATCCTTCTCAAGGTTTGCTAATTCCTCTTGCATAGG | |
| | | | CAATCCGTTCTTCCAGCTTTGTGTTTATCTTTCCAGAGAAGTTTACTGTGTATT | |
| | | | AAGCAAATATGTATATCTTTATTCTTGCTCAGTATTTTCGCAAACAGCAGCTGT | |
| | | | CTAAGTTCACTGTTCTGAACTTTATTTTTTAAATTAAAAATATATGGCTATGTA | |
| | | | GTATTCTATTTTATGGAAGTTCCATATTTCATTTATCCTGTTTCCTTCTACTGAT | |
| | | | GGCTAGTTAGGTTATTGGAAGTCTTTTGCTGTTGCTAGTTAGTCTTGTATAGAC | |
| | | | ATTGTAATGCACATGTGCAAAAATACAAGTATGATACAATCTTAAAGGGGAG | |
| | | | TTGCTGAGTCCAATATATACATTACAAATATTGATAGATATTGCAAAATTGCC | |
| | | | TTCATAGAGGCTATATTAATTTATAGTTCCAGCAGCAACATATGAGTTTATCTG | |
| | | | TTTCTCCATATATATATATATGTATATAACCAACAGACAGTGTTAATTTTTAAA | |
| | | | ATTTTGACAATCTTCTGGGTGAAAGTAGCATTGTATTGTAGTTATCATTTGCTT | |
| | | | TTTAATATTATCATGTAAGTAACAGAGATACTAAACCCAGAAGGATAAGGGA | |
| | | | GCAAAGATGAGAAAAATAAACACACACACAAACAACAATAACAAATCTGTCT | |
| | | | AAAATATTGGAAAATCAGAATGAGAAATGAAATATGACTGTAACGATAAAAA | |
| | | | TCAGTAATAAAAATGACTATTAAATTTAAAAATAAGGCAGAGCAACCACAAG | |
| | | | TGACATGAGAATGAGGCAGACAAAGTTAAAGCACCTAAAGTCTTTGTCTTGTT | |
| | | | TGAAAGGAGGGTAGAGATATTGATTACCTTCAGATGCTGCCACATTTGGTAAA | |
| | | | CATGTTAAAAATATAAGACTGACTTTTAACTAATATAATTAGAATTTAAAATT | |
| | | | CCTAAATCAGTAAGGGGAAATTAAATAATACTTTAAATAAAAATGTAATTGAT | |
| | | | TTAATATAAGTGATGCAAGGAAAACAAAAGAAGCAAAGTAAGACATAGCAA | |
| | | | ATACAGAGCACCATATATTGTAACAGAAACGAATGTAAATAGCACAGTGATC | |
| | | | ACAAGTATAAATAGCTTAAATATGGGAGTTAAAGGAAAGTTCTCAGATTAGA | |
| | | | TTAACAATATCGCAAAATCCAGTTATATGCCCTTCATAAGATCACAAGAAACC | |
| | | | TAAAAACATGAAAATGTTAAATCAAAACCAGATATAAGATACACAAAGCAAA | |
| | | | CACTAACAGAAGACAGAAAGACAGCTTATGTAATTACAGAAATATCTAAACA | |
| | | | TATAGTCTAAAACCAAAGGCATTAGTAAAAACAAAGACAGCCATTACATAGT | |
| | | | GATAATGAAGTCACCAGAACATTATACTAAAACTGAATCAGTTTGCACCTAGC | |
| | | | AATAAAACTACAAAATATGTAAGAAATATAGGAGAATTATAGGAAGAAATTA | |
| | | | ATTGAGAAATTGAAACACATTTCTCAATGATTATAGAATAAGTGGAAAAATG | |
| | | | AATAAGGATATCAAATAAGCCTGTTGATGTTCTTGTCTGTAACATCTAGGAAT | |
| | | | CAAAATATATTCTTTAAAACTAATAAATCAAGTGCTCGTATTTACACATTTAA | |
| | | | GACTACAAAGGTAGACACACACACAGAAAGAGGAAGGGAGAGGATATGAGC | |
| | | | TAGTTTTGTGATTGTTCATTATTTAAAACTAATAGGAATTATCTAAAGGAAGA | |
| | | | GGGAACTAAGTGTATTATATACAAATAAACTTATCAAAGCAACCTTGAAATAT | |
| | | | ACACCTTCCTTAATATCTGAAAAGGTAAAATTTTTAAAATGCACAATAAAGAC | |
| | | | ATAGTGAAAGATTTTTAAAAATACTCATGTAAACATTATACTTAACAGAAGTT | |
| | | | AATATCTCTGAATTAGACTGAATCTATTTGCTGTATGAATAATTATAAATGGG | |
| | | | CTTCTTAACTGAACTAAATAAATGAAAATATTTTTAAATTAGATTAAGATAAA | |
| | | | AACTCTATTATTTGCAATATGCAAAAGACACCTAACATAAAGCCACTCAGAAA | |
| | | | GATGTATTCATTATTTTCAACAAATAAGATGATGGGCAAAGACATAACAAGA | |
| | | | GAAGGCAAACAAAAGGGAAGCAGAGGTCACATTCGTCTTACCAAACAAGATG | |
| | | | AAATTCAGACACTCCTCACCAAAAAAAAAAAAAAAAAAAAAAAATTGAGTGT | |
| | | | GACAAGAAGGGCACTTTATAATGTAAAGTATAAAATTCATATGAATATATGA | |
| | | | GACATGTGTTTAAGTTCTAAATACCAAATAGGTAGCTCTTATAAAGCAGAAAT | |
| | | | TAAGGTAGATACAAGGAAAATATTCAGAAATGTACTACCAATAGAACACTTT | |
| | | | AATTCCCTTTCCCTGGACTAGACCAGTTAAGTGGACAAAAAAATAGGCTGGGC | |
| | | | CTGGTGATTCATGCCTGTAATCTCAGCACTTTGGTAGGCCAAGGCGGGCGGAT | |
| | | | CACCTGAGGTCAGGATTTCGAGGGCAGCCTGGCCAACATGGTGAAACCCTGTC | |
| | | | TCTACCAAAAATACAAAAATTAGCTGGGTGTGGTGGTGGGCGCCTGTAATCCC | |
| | | | AGCTACTTGGGAGGCCGAGGCAGGAGAATTGCTTGAATCTGGGAGGCAAAGG | |
| | | | TTGTAGCGAGCCAAGATCGCACCACTGCACTCCAGCCTGGGCGGCACAGTGA | |
| | | | GACTCTGCCTCAAAAAAAAAAAAAAAAAAAAGTATGAACACAGAAGATAGA | |
| | | | AGATATTACTATCAATAAAAAAATAGATATGCTACATCTATATCAAACTTTTA | |
| | | | AAATTGAAAATAGAGGCCGGGTGCAGTGGCTCACACCTATAATCCCAGCACTT | |
| | | | TGGGAGGCCTAAGCAGTCGGATTGCTTGAACTCAGGAGCTCAAGACCAGTGT | |
| | | | GGGCAACAGCGAAAACCCGTCTCTACAAAAAATACAAAAATTAGCCAGATGT | |
| | | | GGTGGCTCGTGCCTGTGGTTTCAACTACTTGGCTGAGGTGGGAGGATCGCTTG | |
| | | | GGCCCAGTTCAAGGCTGCAGTGAGCTATGATTGTGCCACTGCACTCCAGCCTG | |
| | | | GGCAACAGAGTGAGGCCTTGTCTCTAAAACAAACAAACAAACAAACAAAAAT | |
| | | | TAAATGGCTCAATGGCATAGAAGAAAATTTTTTTATGAAGCAAGTATAATATT | |
| | | | GATGTTAAACTATGACAATGCACAAATATAAAAATTGTAGATGAATTTACTTA | |
| | | | TTATACTGACACAAAAATCCTAAATAAAACATTGCAAACAGATTCTAGCACAG | |
| | | | ATTAAAAAACACCCATTAAGATCAAGGGGTTTTTTTTGTTTTTTTTTTCTAGAA | |
| | | | AACCCTTTGGAAGTTCATGATATTTTGAATTTCAATGGATATTTCCTGGGAATA | |
| | | | ATGAGTTCAAATGAACGAATATGTGGAACAAAGCATCACCAACATTTATTTTT | |
| | | | TCAGGATGAGGTGATGGACAAAACCATCACAGGGAAATTGAGGCAAATAGTA | |
| | | | CATGTAAAACAATACTTCGGGTGAGTCCACCTATCCCAAAGTCGTATCAAAGA | |
| | | | AGTGGCTGCAGATTGGAGCCCAAAGCCTTTGGTTCCTCAGTTTCCAAATGGAT | |
| | | | TCTCACTAGGTGGGATCATGAGTTTGCTTTGGACACCCCAAATTCTAACTATTT | |
| | | | CTTTTGTTTCTTACATCCTTTCCCTCTTCCCCAGCCCCTTCCCCTCATGTTACAC | |
| | | | CTCTTGCTGGTTTGAGACGTCAATCACCACTGAGAAAGAATTAAACCAGTATT | |
| | | | TTGAGCTGGCAAAATTCTTAGCCTAGTACAATTCCTTCAATTAAACTGTAGCTC | |
| | | | AACAATGTGTTCTCTAAAAGTATGATTTAATATTCTACCTAGAAACTCAGAAT | |
| | | | ATTTTTCATAACTCCTCCAGGGCAGTGTGGATTATGTTGATGTGTTAGGAGGA | |
| | | | AGTAGAAGGAAGAAAATGAATTGAGATGTACGTTTTACTTCCATGTCAAAGTC | |
| | | | ACCAAAATAGCAGTTGAAGGGATATTTGTGTCAGGCAATAGGGAGAATAATA | |
| | | | ATTTTATGCCACCCTCAAAAAACAACCACACATACACCCAAATTCTCTCACTC | |
| | | | CTCAGAGAAAGGGGAAGGAAAGAAAAGAGAGAAGCAAAATATGAGCTTGAG | |
| | | | TGAAAAATCACAGAGGAGCCTGTAGCTATTTAGGGAGGGCTGCTGGCTGAAG | |
| | | | TCAGGCAGGAAAAAAAGATCAAAACACCCTGCCCTATTTTTCAGGCTCGTTCA | |
| | | | AGTAGAAGACAAAAACATAAATACAGGAGAAAGGAAGAAAACCACATCTTTT | |
| | | | TCCTCTTGTCTCCCCAGAACTGAACAGTGTCTCTGATAAGCCCAGGCCCTTCTA | |
| | | | TCGTAGACACTGACACTATGCACAGAAAATGACTCAAAAACGCTTCTAATGG | |
| | | | GGGTGAATCTGATGCTTCAGTTTATTTAAGATGTACCAGAGGCCATCTAAGGA | |
| | | | GATCCATAGCTTGCTAACTGAAGCTTATTGCTTTCTCTTCTTAGTTCCCATGCC | |
| | | | AGGCTCTATGCCATTCCTTGCTTGTCATGTGAGATCACTGATTTCCTTTGGTTA | |
| | | | GGTAGGATATGATTTCCTTACAGAGATCTGTCCTGACACCAAACAGCTATGAT | |
| | | | GAAATTCCTTGGTTATTTTCCCTTTTTGTGTACCTTATCATTACCGGAATGCTA | |
| | | | AAGCTGTAGGAATAAAGTTTCCCTGGCTTCCCAAGAAATACAGTGTGACAGA | |
| | | | AAAAGTATAGCCTAGGGACTTACTAGTTATGTGAACTTTGGTTCATTCCATCCT | |
| | | | TACCTCATCTGTAAAAGGGAGATCATGATAGTATCTACTGCAAAGGGTTTTGT | |
| | | | GTGATGATTCTCTCTCTTTCTCTCTCTCTCATACACACACACGCACACATTTAT | |
| | | | ATAATGCTTAGAAAATGAACTCATAATAAGCAATTGACAAACATTAGCTATTA | |
| | | | TTATGTAGGCAAGTCAGATTTTAGAGTTTGTGAGCCTTAGACACATTTACAGA | |
| | | | GAAGAAAGAGCAGCCCTCCTAACTTTCTGGTCCAGCGCCATATCCTCCACTTC | |
| | | | CTCCCCATCCCCACATCCCTTGCCATTTATCAACCCCCTCTCTCCTCTAAATCT | |
| | | | AAATACAGGCCCCCGTTTGATCCATATTGTCTAGGCCCTTCCTCTCCTCTCCAC | |
| | | | ATGGCCCTTCCTTCAGCTCTGAGGGAAGCTGCAGAAGCCAGCCATGGTGCTGT | |
| | | | CTACAAAGAAGGGGACACACGCCTCTTCCACCCGCTCATGCTGTTACTGCATC | |
| | | | TGATCATCTCCGTGCCGTGTCCTTGTTCACTTAGCCTGTGTTGAGTGTTTGTCT | |
| | | | CCATTTCCAAATGCAATAAAACATCTGGGAAAGACTAAGGTAGGTGTGGGCA | |
| | | | GGAAGAAGGGAGGAAGTTAGACCCAGTGGCTTGAGTGCCCTCTGATGCCTCC | |
| | | | TTATCCTCGGCTCCACACAAGCCCTCGCCAGTGTGAGCTCCACAGCCATCCAC | |
| | | | CTGGAGGAGGAGTACTCAAAACCAGGGTCAAATGCCTTGTACTCGGGGGTCT | |
| | | | ACCAGTAAGCCTGTGGCCAGCCTTCCACTTCATGAATCGTCACATTTACATGA | |
| | | | GAGATGTGGAGGGAGAGGGGTCAGCCTCCTAGCTCCTGTCCTGTAGCAGTTAA | |
| | | | GTCAAGTCAGGCCAGGCTGTGGTAACCAAGCCAGTACCTTCTTTAAAGAACAG | |
| | | | TTGTTTCTCATTCATACAGAGTGTGTTGTAGGTGTGAGTGATTCCCTGGGGCAG | |
| | | | CTGCTCTTCATGGTGACTCAGCTAATTAGACTGCTTTTCTACTGGGATTGTGCC | |
| | | | ATCTCAACACAAGGCTGTCTTGGTTGACTTGGTAGGGGAGGAGGAGAAGAGT | |
| | | | CATGCACGAGGAGTTAAATTCTTTGACCAGGAACCTTTTGTTCACTTTCCATTG | |
| | | | GTGAGAACGAGTCACACAGCTCCAGGAGAGTAGGAAGTCTAGTTTCTCGTTTT | |
| | | | CCATGGGCCTAGGAATTAGAGGATGAACACTAGCGATGTCTTCCACGTCTTGC | |
| | | | TCTCTTTTTCTTTACCTGTTATAATCTCCCATGGAGAAGAAATTATTTTTCTGAT | |
| | | | TGGTCTGATCCAGGCCTCTAACCAAGGGCATTTACTAAGGGTCATAGAACAGT | |
| | | | AAAAGGTTGCACAATTGCATCTCACCTCTCTTTTCTGAATCCTGCTTAATTAAA | |
| | | | ACTTTAAAAACTCACTTACAGACCACTTCTTGCTCTTCTTAGTTATCAAATATA | |
| | | | TTTTCTTCCAACTCCTTTTTGACAGATCTTGCCTTGTTCTCAGCTGACTCCCTTG | |
| | | | CAACCATCTAAATGGAACTTCCTCATTCCCCTTTATCCCCTGCTAATCGTCCTG | |
| | | | TCTTTATTCTTTTATCTGAAGAAGTGTACCCTTCTTCCCCCAAGCCTTATGCTCT | |
| | | | CTGTCTTAGTCCATTTGTGTTAGTGTAACAAGATACACGAGATAGGGTAATTT | |
| | | | ATGAAGAACAGAAATTTATTTCTCACAGTTCTGGAAGCTAGAAGTTCAAGGTC | |
| | | | ATGGCCCTGGAAGATTTGATGCCTTGGCCATCGACCTGAAAAAAGTCACTGAA | |
| | | | GTGAACTTGTGGTTACCATGATACCCTGGGTTCATTCTTAACATGCACAGTAT | |
| | | | AGGCAAATCAGGGGCACATTTCATGCTGAAAGATGATGTGAAATGATGAGTA | |
| | | | AACATTGCCCTTTAAATAATAATAACATAGATGGTGAATTACAATTTGCACTT | |
| | | | TTGGTTTTATTTGCTTAAAGATAGTGTTTTACTGTCTAGGTTTTACTGAAAGAA | |
| | | | TACTTAACTAAACACTTGAGTCAAATCATTCCAGTGTAACAGTCCTCACAAGG | |
| | | | AGGAAGGCAGAAGGGCAAAAGTGCCAAGCTATTGCCTCCAGCCCTTCTATTTT | |
| | | | TATTGATTGACTGATTGATTGATTGAGACAGGGCCTCGTTCTGTTACCCATGCT | |
| | | | GGAGTACAGTGGTGCATTCATGGTTCACTGCAACTTTGAACTTCTGGGCTCAA | |
| | | | GTAGTCTTCCCACCTCAGCCTCCAAAGTAACTAAGACCACAGGTGCATGCCAC | |
| | | | CATGTTTATTTCTTTAATTTTTTAAATTTTATTTGGACATGGGGGTCTCACTATG | |
| | | | TTTTCCAGGCAGGTCTCTAACTCCTAGCCTCAAGCAATCTTCCTCCTCAGCCTC | |
| | | | CCAAAGTGCTGGGATTACAGGCATGAGCCATCATCCCTGCTCTCTCTAGCCCT | |
| | | | TTTTAAAGTCTCTAATCCCATTCAGGAGGCCTCTGCATTCATGACTTAATCACC | |
| | | | TCCTAAAGGGCCCACCTTTTGATACTATCACATTGGTGATTAAATTGCAACAC | |
| | | | TTGAATTTTGAGTGACATTCAGACCACAGCATTTTGGAGAAAATGTCCCCTTT | |
| | | | CTTCTCTTTTTTCTCTTCAGTTTCTCCTTTCTACAGATACCTTGCACTTCACTTG | |
| | | | CAACCTTGTTGAAGTCTCTCCTGTCTTAAAAGTTTCTTTTCCCAAAACACCAGA | |
| | | | ACTCATTCCTTCTGTCTAACTGTAACTTTGTACTTGTTACCAACCTCTACCCATT | |
| | | | TCTACCCCTGACTCCCCAGCCTCTGGTAACTCCTAACGTACTCTTTACTTCGAT | |
| | | | GAGGTCAACTTTTTTAGATTCCACATATGAGTGAAATCACGTAATATAGTCCT | |
| | | | TCTGTTCTTGGCTTATTTCACTTAACATAGAGCCCTCCAGGTTCACCCATGTTG | |
| | | | TTGAAAATGACAGGATTTCATGCTTTTTTATGGCGGAATAGTATTCCATCATGT | |
| | | | ATATATATCACATACTAATTACCCTGATTTGATCAATACACAATGTATAGGTG | |
| | | | TGTTGGAACATCATGCTGTACCCCATAAATATGTACAATTATTACGTGTCAATT | |
| | | | TAAAAAACCCAAGAAAAGTATTTTTTCTTAGTGTTGTTTTTTTCTAAAGTGTAT | |
| | | | CCCATTTTTTCCTTTGTTTTCACTTTCAAAATTCTTGAAAGACTGTTTTAATCTG | |
| | | | CGGCCTTTGTTTTCTTGTTTCCAATTCATTCTTTTGAAATATGGCTTCTACCTTT | |
| | | | AACACTCTGCTGTCCTCTTGAAGGTTATTGGTGCCCTACTAACCATAAAACAC | |
| | | | AATAATGCTCTCTTAGATTGTATCCTGGTGAGCCTTTTTGCAAAATTTTATTCT | |
| | | | TTGAGAATTCTCTTTCCATGTTTTCTTTTCTTTTCTTTTTTTGTGGTCATTTTGGC | |
| | | | TTATAGGAAATCGTACTCTCCAAATGTTCTCTATGTCTTCTATTTCTTCTCTGAT | |
| | | | AATCCCTTTACTAAATCTTCTTGAGGCATATGCTTCAGAATGCTTAATTTACAT | |
| | | | ACTCTTAATTCCTTCTTAATTTACACACTGCCTGTCGGCAATGTCAACACCCAA | |
| | | | TAAGAAGGGAGACCTATTGGTCAGAACAGGCCAGGGAATAGAAGAATACATA | |
| | | | ATAAACAGTCTGCCTAGTTCTTCTAGGGCCCCATAATATGTCAAACATATATTT | |
| | | | TTACTTCTTCTCCCAGCCCATTTTTAGTATACCTAAATTACTGTCAGTGATTCT | |
| | | | GCAGGCAAACAATGGTTGAGTTGTATGACACAACTTTGTGAAAGTATCCTACC | |
| | | | CAGTACCTGATGATGCAAAACTCTIVTATCTTGATTGGTTGTCAATCTGAGGA | |
| | | | GTTTCCAATCCTGGGGAAGCCAGAAAAACAGCGATTTATACTCTTAATGGGTA | |
| | | | CTTTCTGACTGAATTTTATGAGCTCATTCTGAAGAGGCTGACGATTTTACTATC | |
| | | | TCATTTTTTTCCTTTCTCCAGAATGGGTTCTGGGTGGGTCCCCTGGGTGGTGGC | |
| | | | TCTGCTAGTGAATCTGACCCGACTGGATTCCTCCATGACTCAAGGCACAGACT | |
| | | | CTCCAGGTAAGAACAGAGCAATTGTTTTTTTCCAGTGTGTATGCAAGAATTGG | |
| | | | CATGGGGGAGTGATGCCTTTCTTTGTAAGTCCAGGCCACAGACCAGACTGGAA | |
| | | | GTGGCTTTTGGTTTCAAAGAACAGTGTTCTTCCCTTTGGCAGAAAGGTACGCC | |
| | | | TTGCCTCTTTACATGGGATGGACTTCATATACCAGAGCCACCTATTCAAGGGG | |
| | | | TAGGGAGGCAGGAAGAGGGAAACATTGTGTCTTGTTTAGGATCCTTATTGTGT | |
| | | | GTATCAACCTCAGTCAGTGCCTGGGCGTGTTGAAGGCCTTGGCTTGGGTTCGA | |
| | | | GCCTGCTGGGAGAAACAACCTGCAGTAGGCTGGGTCACAGAGGCAATCTGTG | |
| | | | ATTTTTTGGTCAGGACACGGAAACAAATCTCAGTTGGGGTATATGTGGACAAA | |
| | | | TGAAACTGGAAACAAAGGTTGCTCCTTCTGTCATTTATTAAGCCACTATTATAT | |
| | | | TGTCAGAATTGTACTAAACAGTTTTGAGAAGTAAGAGAAGTTGAATAGAATA | |
| | | | CATTGTCCTTGTCCTCCGGCTACCAGGTACAAGTTACTTGTCACTGTTATTTTT | |
| | | | CTAGCACAGGTGACAGAATATGCAGCCATGAAGCAATGTGAGATGAAAGCAC | |
| | | | ATATTAATGAGCAGAAACAGGATGTAATGTGCTAAGAACAGAATCCCCTTTGC | |
| | | | ATGTTAGTTTCATTAAATACAAAAGAGGAACAAACCTGGCCAGGAGAGATCA | |
| | | | TTATTCTCAGAGAATAGAAACCGCCCTGAGTTTATAATGTCCATTAAACAATA | |
| | | | CAACTGAAAAAAAAATCAGCACAGATGTTAAATGATGATGAAAAATTCAGAT | |
| | | | TTCCCCCCTGGTTTAGACTACTAGAGGAAATAGAGAAGAGTATACATGCTGAG | |
| | | | AAATTACAGGCTGGAACTTCATCTGAAATTAGCTACTGAGTGAGGGATAAGTG | |
| | | | GGGTTCACCCAGGAAGGTCATTCTTATGGCTCAGTTCAGAGTTGGAGGAGGCT | |
| | | | TCTGAACTTAGAAAGGAAAGTAAATTACAACCCAACATTAATAGCAATTATCT | |
| | | | TTCAAGTCTTGACTTAGATGCAATGTCTTCAGGACGTCCTTCCTGACTTACCTA | |
| | | | CATTATTAACTCCATTTGAATTTCCTTTTTATTGTAGTTGTTGTTCTTAAGTGCA | |
| | | | TAGGATTGGTTTAATTTTACCCAATGAGTTCACAGCACATTGTAATTATTGGCA | |
| | | | GTAGTGCAAGACTCTCTCGTCTCTTCTCTTGCCTCCGTTCTCATTCTCTCCCCTC | |
| | | | CCTAGAGAATCCATTCTAAACGTGTCTGGTAAGTCTCTAAGTATGAGAGTGGC | |
| | | | TTTTAGAAATATGTAGCATTATTGCTCTTTATGTGTTTTTAAAATTTAATAAAT | |
| | | | GTCATTCTCTGTTGAATCCCATTCTGTTTCTTTTCTCACTTGACACTGTGCTTTT | |
| | | | TGAGCATACTGAGGTCAAAGGCCTCCTCTTAGATCCATGTGGTCTGACGTAAT | |
| | | | TTACCAGGCATGGGTTTTCCCAGAGGAGGGGGCTGGTTCATGGTTTTGGTTTT | |
| | | | GGTTTTCCAGAAGATTTTGTGATTCAGGCAAAGGCTGACTGTTACTTCACCAA | |
| | | | CGGGACAGAAAAGGTGCAGTTTGTGGTCAGATTCATCTTTAACTTGGAGGAGT | |
| | | | ATGTACGTTTCGACAGTGATGTGGGGATGTTTGTGGCATTGACCAAGCTGGGG | |
| | | | CAGCCAGATGCTGAGCAGTGGAACAGCCGGCTGGATCTCTTGGAGAGGAGCA | |
| | | | GACAGGCCGTGGATGGGGTCTGTAGACACAACTACAGGCTGGGCGCACCCTT | |
| | | | CACTGTGGGGAGAAAAGGTGAGCTGGAAGCTGAGGTCTGGCGGGGCTCAGGA | |
| | | | ATGTCCCCCATGTGAACCTGGCCATGGCTCTTCTTTCTTACAAGCAATTTTCTG | |
| | | | CTTTAGGATAAATGGTTGTCTGTGTAGATGTTCTGGCCCCAGCTGTGATATATT | |
| | | | ATCCTCACAAGTCAGCCACTGTGATCTTGGTCTCAGACCCCCAAGGTTCTCAG | |
| | | | GGACTTCGAGGGCTATTGTGCCCTCAAAGAGAAGCAGTAATTGTGGGAGTAC | |
| | | | CTCAGAAAGTCTAAATCCTCCTGACAGGCATTGACATACCCTGTTACTGATCT | |
| | | | TGGGGGCTGAGACTTGCCTATACTTTGTGTTCACTTGGGTGATCTGGGAAAGA | |
| | | | GATTAGACATAGTGATAGTCCCTAAAGAATCTCCTGTCCCAGCTTGGTGGTTT | |
| | | | TCTTTCACGGTGTCTCATTTTTCCTCCCTTCCTAGTGCAACCAGAGGTGACAGT | |
| | | | GTACCCAGAGAGGACCCCACTCCTGCACCAGCATAATCTGCTGCACTGCTCTG | |
| | | | TGACAGGCTTCTATCCAGGGGATATCAAGATCAAGTGGTTCCTGAATGGGCAG | |
| | | | GAGGAGAGAGCTGGGGTCATGTCCACTGGCCCTATCAGGAATGGAGACTGGA | |
| | | | CCTTTCAGACTGTGGTGATGCTAGAAATGACTCCTGAACTTGGACATGTCTAC | |
| | | | ACCTGCCTTGTCGATCACTCCAGCCTGCTGAGCCCTGTTTCTGTGGAGTGGAGT | |
| | | | GAGAATTAGTTTCTAGTACTCTCTGGGCCTGACTCAGGACTATACTGACTCAA | |
| | | | TACAGAGCCTGTGTCACTTCTGCGTTTATCTTGGTCACAACATGAATTATTCTT | |
| | | | TCCCTTGATCTGGGACAGTCACAGAAACCAGAGTCCTTGGGTTAGGGTGGGAG | |
| | | | AAAACATGGCAGATATCTATCCTCATATCTTCCAAGAAATGAGGAGATCTAAT | |
| | | | CACCTCATTATGTGCTTCCAACCCTATGAACTGGTGTCCTCTAATTCTTTGGTC | |
| | | | TTAGTATTTAGGAGGCATTCTTATGGGCTGTGAGAATCTGTAACCGATGGGGG | |
| | | | GTAACTCCATGGGTGCCAACTTTGGTTTCGAAGAACCTTTTCTAAATTTATTTA | |
| | | | TTTTTCTCTAGCTAGCATTGGATTTGGTGTCTAGTACAGATTCTGGGATTCCAA | |
| | | | GAAAGTGCTTTAAATATTGGGATATTTTTACTAATTTAAAGACCTGTTTCCCAT | |
| | | | AGGAGCTCAGTCTGAATATTCTTGGAGAAAGATGCTGAGTGGCATTGCAGCCT | |
| | | | TCCTACTTGGGCTAATCTTCCTTCTGGTGGGAATCGTCATCCAGCTAAGGGCTC | |
| | | | AGAAAGGTAATGAGCCTGTGAGGAGTGCCCTGCCACCTGTCCCAGACCTTCCC | |
| | | | CACTCCCACCTTCCCTAACGTCAATGATCTGAGGCAAGGAAAGCTGATTGTGC | |
| | | | CTCTCAGGGATCACCGGGATAATTTTTTTCTGAAGCTAGAAATGGGATAAGCA | |
| | | | GAGAGAGTGCTGACCTTGCCAGCCATTTGTTCTTCCCTCGGGATAATCATATT | |
| | | | GGGTCCTAATTGGGGCAATCCATTCTTTTCTCGATTTCTTTCCAGGATATGTGA | |
| | | | GGACGCAGATGTCTGGTAATGAGGTAATGTCTCTTTTTCCTTGTCTTTGAGTGG | |
| | | | CAGATCATTCTCCCGGTTCTTTGGCCAGAGGGAGATGACATGGGGGTAGGGA | |
| | | | GGAGTAAGGTTGCTGCTGTCTGGATGGGACTGTCCCCTGAGTCTCTGGAACGG | |
| | | | CTGTGGGGGGTGGTGAGGCTGCCTCCTGAGACCTTCATCACTGTGCCTCCAGG | |
| | | | TCTCAAGAGCTGTTCTGCTCCCTCAGTCATGCTAAGGTCCTCACTGAAGCTTCT | |
| | | | CTCTCTGGAGCCTGAAGTAGTGATGAGTAGTCTGGGCCCTGGGTGAGGTAAAG | |
| | | | GACATTCATGAGGTCAATGTTCTGGGAATAACTCTCTTCCCTGATCCTTGGAG | |
| | | | GAGCCCGAACTGATTCTGGAGCTCTGTGTTCTGAGATCATGCATCTCCCACCC | |
| | | | ATCTGCCCTTCTCCCTTCTACGTGTACATCATTAATCCCCATTGCCAAGGGCAT | |
| | | | TGTCCAGAAACTCCCCTGAGACCTTACTCCTTCCAGCCCCAAATCATTTACTTT | |
| | | | TCTGTGGTCCAGCCCTACTCCTATAAGTCATGATCTCCAAAGCTTTCTGTCTTC | |
| | | | CAACTGCAGTCTCCACAGTCTTCAGAAGACAAATGCTCAGGTAGTCACTGTTT | |
| | | | CCTTTTCACTGTTTTTAAAAACCTTTTATTGTCAAATAAAATGGAGATACAAAA | |
| | | | AATGTACATTTTAGTGAATTATTTAAGAAAAACCCCTGTAATCAAGTCAAGGA | |
| | | | ACAGGACTTTGCCAGCTCCAGCGGAAGTCTCTGTACGTCAGGCCAATCAAAGC | |
| | | | CTCTCCTTCCCCTCGAAAGTGACCATATCCTGATTTTATTGTAACCTCTTTCAT | |
| | | | GTCTTTGTAGTCTAGTCCCCCAGGTATGTGTTCCTGGACGCCACAGCTTAGTTG | |
| | | | TCTTTTACGTCTCTCATACTTCACTGGTTTCTCCCCCATCAGCCTTTTTTTCCCC | |
| | | | CTTATGATTAATCTGTTGAAAAGCTCAATCCATTTGACCTGTAGAGTTTCCCAC | |
| | | | ACTCTGGATTTTGCTGGCTGCGTACACATGGTGCAGTGTAACACATCCTCTGC | |
| | | | CTTCTGTGCCTCCCGCAAATTGGCATCTGGATGCAGAGGCTGGATCAGACTCT | |
| | | | GGTTCAATCTTTTCCTTTGGAAAATCTATACGTGGTGTTGTGGCCTTTCACCAT | |
| | | | GAGAGACATAATTTCCAGCTGTTTCTTTTTGTGATGTTAGCAACCATTGATACT | |
| | | | AATTGCTTAGGTCTGTTAATTTATTGGGGATTGCTAAATGGTGGTATTCTGTCA | |
| | | | TTTTTTCTTCATTTATATGCTTTATTAATGCTACAAAGAGACAGTTCCCTTCATT | |
| | | | TACTAGTGAAGAGACACTTCCCTCCACTTACTAGTGGGCTACTTAGTGGTACA | |
| | | | GTGGAAAGGCAGGATCAGTGTTTGACTCTCCTTTTATTTACCAGTTTTCAAGTT | |
| | | | AGCAAATTGGTTTCCTACTATTAATAAAACCTACAGATGGCCAATCAGGTTTT | |
| | | | ACTTTTCATTCTTTAAATAATTATAAACAGAAGGATTTAAATGTTTGATAGGTT | |
| | | | TTACTCTATTGCAATTTTTATGCTTGTTAAAGCTTATATCCTACATTTTTGGTGA | |
| | | | GTGAAAACTTCTTCAAGTGGCTAAAAATGAAACTGAGTTCTTTTGACGCAAAT | |
| | | | TTCTTTTGATAATTTCTTTGTTATCTGGGATGAC | |
| 53 | HLA-E | >NP_005507.3 HLA | SEQ ID NO: 103 | |
| | | class I | >NM_005516.5 Homo sapiens major histocompatibility complex, class I, E (HLA-E), | |
| | | histocompatibility | mRNA | |
| | | antigen, alpha chain | GCAGACTCAGTTCTCATTCCCAATGGGTGTCGGGTTTCTAGAGAAGCCAATCA | |
| | | E precursor [Homo | GCGTCGCCACGACTCCCGACTATAAAGTCCCCATCCGGACTCAAGAAGTTCTC | |
| | | sapiens] | AGGACTCAGAGGCTGGGATCATGGTAGATGGAACCCTCCTTTTACTCCTCTCG | |
| | | MVDGTLLLLLSEA | GAGGCCCTGGCCCTTACCCAGACCTGGGCGGGCTCCCACTCCTTGAAGTATTT | |
| | | LALTQTWAGSHSL | CCACACTTCCGTGTCCCGGCCCGGCCGCGGGGAGCCCCGCTTCATCTCTGTGG | |
| | | KYFHTSVSRPGRG | GCTACGTGGACGACACCCAGTTCGTGCGCTTCGACAACGACGCCGCGAGTCCG | |
| | | EPRFISVGYVDDT | AGGATGGTGCCGCGGGCGCCGTGGATGGAGCAGGAGGGGTCAGAGTATTGGG | |
| | | QFVRFDNDAASPR | ACCGGGAGACACGGAGCGCCAGGGACACCGCACAGATTTTCCGAGTGAATCT | |
| | | MVPRAPWMEQEG | GCGGACGCTGCGCGGCTACTACAATCAGAGCGAGGCCGGGTCTCACACCCTG | |
| | | SEYWDRETRSAR | CAGTGGATGCATGGCTGCGAGCTGGGGCCCGACGGGCGCTTCCTCCGCGGGT | |
| | | DTAQIFRVNLRTL | ATGAACAGTTCGCCTACGACGGCAAGGATTATCTCACCCTGAATGAGGACCTG | |
| | | RGYYNQSEAGSH | CGCTCCTGGACCGCGGTGGACACGGCGGCTCAGATCTCCGAGCAAAAGTCAA | |
| | | TLQWMHGCELGP | ATGATGCCTCTGAGGCGGAGCACCAGAGAGCCTACCTGGAAGACACATGCGT | |
| | | DGRFLRGYEQFAY | GGAGTGGCTCCACAAATACCTGGAGAAGGGGAAGGAGACGCTGCTTCACCTG | |
| | | DGKDYLTLNEDL | GAGCCCCCAAAGACACACGTGACTCACCACCCCATCTCTGACCATGAGGCCA | |
| | | RSWTAVDTAAQIS | CCCTGAGGTGCTGGGCCCTGGGCTTCTACCCTGCGGAGATCACACTGACCTGG | |
| | | EQKSNDASEAEHQ | CAGCAGGATGGGGAGGGCCATACCCAGGACACGGAGCTCGTGGAGACCAGGC | |
| | | RAYLEDTCVEWL | CTGCAGGGGATGGAACCTTCCAGAAGTGGGCAGCTGTGGTGGTGCCTTCTGGA | |
| | | HKYLEKGKETLLH | GAGGAGCAGAGATACACGTGCCATGTGCAGCATGAGGGGCTACCCGAGCCCG | |
| | | LEPPKTHVTHHPIS | TCACCCTGAGATGGAAGCCGGCTTCCCAGCCCACCATCCCCATCGTGGGCATC | |
| | | DHEATLRCWALG | ATTGCTGGCCTGGTTCTCCTTGGATCTGTGGTCTCTGGAGCTGTGGTTGCTGCT | |
| | | FYPAEITLTWQQD | GTGATATGGAGGAAGAAGAGCTCAGGTGGAAAAGGAGGGAGCTACTCTAAG | |
| | | GEGHTQDTELVET | GCTGAGTGGAGCGACAGTGCCCAGGGGTCTGAGTCTCACAGCTTGTAAAGCCT | |
| | | RPAGDGTFQKWA | GAGACAGCTGCCTTGTGTGCGACTGAGATGCACAGCTGCCTTGTGTGCGACTG | |
| | | AVVVPSGEEQRYT | AGATGCAGGATTTCCTCACGCCTCCCCTATGTGTCTTAGGGGACTCTGGCTTCT | |
| | | CHVQHEGLPEPVT | CTTTTTGCAAGGGCCTCTGAATCTGTCTGTGTCCCTGTTAGCACAATGTGAGGA | |
| | | LIQNTYASQPTWPV | GGTAGAGAAACAGTCCACCTCTGTGTCTACCATGACCCCCTTCCTCACACTGA | |
| | | GHAGLVLLGSVVS | CCTGTGTTCCTTCCCTGTTCTCTTTTCTATTAAAAATAAGAACCTGGGCAGAGT | |
| | | GAVVAAVIWRKK | GCGGCAGCTCATGCCTGTAATCCCAGCACTTAGGGAGGCCGAGGAGGGCAGA | |
| | | SSGGKGGSYSKAE | TCACGAGGTCAGGAGATCGAAACCATCCTGGCTAACACGGTGAAACCCCGTC | |
| | | WSDSAQGSESHSL | TCTACTAAAAAATACAAAAAATTAGCTGGGCGCAGAGGCACGGGCCTGTAGT | |
| | | | CCCAGCTACTCAGGAGGCGGAGGCAGGAGAATGGCGTCAACCCGGGAGGCGG | |
| | | | AGGTTGCAGTGAGCCAGGATTGTGCGACTGCACTCCAGCCTGGGTGACAGGG | |
| | | | TGAAACGCCATCTCAAAAAATAAAAATTGAAAAATAAAAAAAGAACCTGGAT | |
| | | | CTCAATTTAATTTTTCATATTCTTGCAATGAAATGGACTTGAGGAAGCTAAGA | |
| | | | TCATAGCTAGAAATACAGATAATTCCACAGCACATCTCTAGCAAATTTAGCCT | |
| | | | ATTCCTATTCTCTAGCCTATTCCTTACCACCTGTAATCTTGACCATATACCTTG | |
| | | | GAGTTGAATATTGTTTTCATACTGCTGTGGTTTGAATGTTCCCTCCAACACTCA | |
| | | | TGTTGAGACTTAATCCCTAATGTGGCAATACTGAAAGGTGGGGCCTTTGAGAT | |
| | | | GTGATTGGATCGTAAGGCTGTGCCTTCATTCATGGGTTAATGGATTAATGGGT | |
| | | | TATCACAGGAATGGGACTGGTGGCTTTATAAGAAGAGGAAAAGAGAACTGAG | |
| | | | CTAGCATGCCCAGCCCACAGAGAGCCTCCACTAGAGTGATGCTAAGTGGAAA | |
| | | | TGTGAGGTGCAGCTGCCACAGAGGGCCCCCACCAGGGAAATGTCTAGTGTCT | |
| | | | AGTGGATCCAGGCCACAGGAGAGAGTGCCTTGTGGAGCGCTGGGAGCAGGAC | |
| | | | CTGACCACCACCAGGACCCCAGAACTGTGGAGTCAGTGGCAGCATGCAGCGC | |
| | | | CCCCTTGGGAAAGCTTTAGGCACCAGCCTGCAACCCATTCGAGCAGCCACGTA | |
| | | | GGCTGCACCCAGCAAAGCCACAGGCACGGGGCTACCTGAGGCCTTGGGGGCC | |
| | | | CAATCCCTGCTCCAGTGTGTCCGTGAGGCAGCACACGAAGTCAAAAGAGATT | |
| | | | ATTCTCTTCCCACAGATACCTTTTCTCTCCCATGACCCTTTAACAGCATCTGCT | |
| | | | TCATTCCCCTCACCTTCCCAGGCTGATCTGAGGTAAACTTTGAAGTAAAATAA | |
| | | | AAGCTGTGTTTGAGCATCATTTGTATTTCAAAAAAAAAAAAAAAAAA | |
| 54 | CCL4 | >NP_002975.1 C-C | SEQ ID NO: 104 | SEQ ID NO: |
| | | motif chemokine 4 | >NG_033066.2 Homo sapiens C-C motif chemokine ligand 4 (CCL4), RefSeq Gene on | 134 |
| | | precursor [Homo | chromosome | NG_033066.2 |
| | | sapiens] | 17TCACTTCTCTAGGTTTTGCTTTCCTCTTCTGTACCGTGGCAAAACAGATTGGG | |
| | | MKLCVTVLSLLM | ATCAGGTAAAACCACAGGCCATGGATATACTGAGGAAAATGAAAAAATACCA | |
| | | LVAAFCSPALSAP | GGACATTTTCAAGGTCATGTTACTGCTGTGGCTGCTGGGCCTGTCTGCAGCCT | |
| | | MKISDPPTACCFSY | GGGAGGTTGAGGACAACATGACAAGACAAATGTAAAAATGCCCAGAATGGA | |
| | | TARKLPRNFVVDY | GTTATGAATATAGTGGTGGTCAAGGATGTGGAGGGGGAACCTCTAAATGACC | |
| | | YETSSLCSQPAVV | ACAAGTGAGGGGGAAAGAAGGGCTTCCCTGCTCAGGGTGAGGGGGTGGACCT | |
| | | FQTKRSKQVCADP | CTGAATGGAGCTCCCTGGAACCAAGGATGACCCTTGCTGTCACTTCTTGCAAC | |
| | | SESWVQEYVYDL | CCTGAGCTAAAAACAGTTGAGTCCAAGAATAGTATCCATTGTGATAAAACTAC | |
| | | ELN | TGGATGCGTGGGTTGAGGGTGGGGGAGAAGGGGAAGTACCACACTATCAGGG | |
| | | | CTGAGGAATCACCTTTTGGGTGGAATGTCAGAACACCCAGTTCATTCATACTT | |
| | | | GGTTTACTCATTTGGCTATATAGGGGTCCACATCTTGAGTCCATGTTACTCATC | |
| | | | ACATTCCCTCCATGGAGATAAGCATAAACAGTTTCTGCATCAAGGAGCTCACT | |
| | | | TTCTAGTTGGGGAGGCAACCACAGGAATAGATAAATATGTCATAGGTGGTAA | |
| | | | GAAGAAATAGTTCAGGAGACTGTAGGAGCACAGTGAAGGATACAGAGTTTAC | |
| | | | TTGGGAAAGAGGTCAGGAAAGTTTCTCTAGGGAAGATGGGAATCTTTTTCGTC | |
| | | | ATGTATCTGAGAGTCATTTGTTATCATTGATTATTGGCTCTCCTTGGCTTTCCA | |
| | | | GTCTCCACATTTGCCCTCCAGGAGTTCTAAATGGCACCAGTGCCTAAATCTTG | |
| | | | GGGTGGGGGAAGTTAATATGGCTAAACGAAATGATACAAGTCCTTTTTACTCT | |
| | | | CAGTGTGGACTTCCACTGGTGCAGACATAGTATTGTTGTTAGGATCTTCATGA | |
| | | | AGATCTCTCAAAACCTTTTACCCCCATGAGCCAAAGATCAGATCAAGGCGAGG | |
| | | | AAACAAAAGTCAACAGACAAACTCATTGTATGGCTCAGTGATGACTAATCAC | |
| | | | CCATTTTATCAATCATTATTCTCCTGAAGCCCCAGAACAGGCTTCCTCCTAACC | |
| | | | CCTCCACCCTGCTCATTCAGACAGCTGCTTTAGTGGGGATTGAGACTTAACTT | |
| | | | GAAACTACTCATCTACAGCTAATAGACAGCCTTCTCCATCCTTCAGCCTTCCTG | |
| | | | ACACTTTGTTTCTTCCTCTGACATGACACTTAGCTTATTCCTCCTCATGGGATA | |
| | | | GTTATTTCTGTCACTGTCAATGCCATCACTAGACATGGAACTCTTCGAGGGCA | |
| | | | GGGATGGGGCTTCATTCAGCTCTGTCTGCCCTGGCAAAGAGCAGATGGTTAGT | |
| | | | ACATTGGGTTGAATAAAAATGTCATAGATAACGAAGTCTGTGTTAATCCAGAG | |
| | | | GACATGAGACACATTCTGCTTCCCTTAAAGTGAGGTCCTCGATGCATGAAGAT | |
| | | | GAAACTGCACCAACTAGGAAAGGCCAGGAGAGGTACTAACACATCTGCTATC | |
| | | | AGGGAACTCGGAACATTACAGCTGGGGGCGAGGGTCTTGGAGAAGATCAGAA | |
| | | | CATTTGAAAAAAGAGCTGTACAAAGAAAAGGGGGAGAGAACAGATGAGAGA | |
| | | | AGTGCAGCTTGTGTGTGCGGGTGGGGAGTGAGGGAGAGTGCTGAGGCAGGAT | |
| | | | GACCTTGTACTTTATTTAATGACCCTGAAGTACCCCAGGATGCTGCTCCAACCT | |
| | | | CTCCTATGGCTCCCAGATGCTGTGGGTCCCTTYTTTTCTTAACTTGTATTTTAGG | |
| | | | TTCAGGGGTACATGTGCAGGTTTGTTATATAGGTAAGTTGTGTGTTGCGGGGG | |
| | | | GTTTGGTGTACAGATTATTTCATCACCCAGGTAATAAGCATAGTACCCAATAG | |
| | | | GTAGTTTTTTTTATCCTCACCCTCCTCCCACCCTTCACCCTTAAGTAGGCCCTG | |
| | | | GTGCCTGCTGTTCCCTTCTTTGTGTCCATGTGTACTCAATGTTTAGCTTCCACTT | |
| | | | ATAAGTGAGAACATGCAGTATTTGGTTTTCTGTTCCTGTGTTAGTTCGCTTAAG | |
| | | | ATAATGGCCTCCAGCTCCATCCATGTTGCTGCAAAGGGCATGATCTTGTTCTTT | |
| | | | TTTTTTTTTTATGACTGAGAAGTATTCCATGATATATATATATATATATCCCAA | |
| | | | ATTTTTTTTATCCAGTCTACCATCAGTGGGCATTTAGGTTGATTCCATGTCTTT | |
| | | | GCTATTGTGAATACTGTTGCGATGAACATACATGTGCACATGTCTTTATGGTA | |
| | | | GAATGATTTATATTCCTTTGGGTATATACCCAATAATGGGATTGCTGGGTTGA | |
| | | | ATGGTAATTCTGTTTTAAGTTCTTTGAGAAATCGCCAAACTGCTTCCCACAATG | |
| | | | GCTGAACTAATTTACATTCCCACCAGCAGTGTATAAGTGTTCCTTTTCTCCACA | |
| | | | GCCTCACCAACATCTGTTATTTTTTGACTTTTTAAAAATAGCCATCCTGACTGG | |
| | | | TTTGAGATGGTATCTCATTGTGGTTTTAATTTGCATTTCTCTAATGATTAGTGG | |
| | | | TGCTGAGCATTTTTTGTATGTTTCTTGGCCGCGTGTATGTCTTCTTTTGAAATGT | |
| | | | GTCTGTTTATGTTCAATGCCCACGTTTTAACGGAGTTGTTTGTTTTTTGCTTGTA | |
| | | | CATGTGTTTAAATTCCCTATAGATTCTGGATATTAGACCTTTGTGAGATACATA | |
| | | | GTTTGTAAATATTTTCTCCCATTCTGTAGGTTATCTCTTTACTCTGCTGATAATT | |
| | | | TCTTTTGCTGTGCAGAAGCTCTTTAGTTTAATTAGATCCCATTTGTCAGTTTTTG | |
| | | | GTTTTGTTGTGATTTGTTTTTGGCATCTTCATCATGAAATATCTGCCAGATCCT | |
| | | | ATGCCCAGAATCATATTGTCTAGGTTATCTTACAGGTATCATATTTTTATAGTT | |
| | | | TTATATTTTAGGTTTTACATTTAAGTCTTCAACCCATTGTGAGTTGATTTTTACA | |
| | | | GATGGTGTAAGGAAGGGGTCCAGTTTCAATCTTTTGCATACAGCTAGCCAGTT | |
| | | | ATCCCAGTACCATTTATTGACTGGGAAGTCCTTTCCCTGTTGCTTGTTTTTGTT | |
| | | | AACTTTGTCAAAGTTCGGATGGTTGCAGGTATGCAGCATTATTTCTGAGCTCTC | |
| | | | TATTTTGTTCTATTGGTCTATGAGTCTGTTTTTGTACCAAAGCCATGCTGTTTTG | |
| | | | GTTACTGCAGCCCTGTGGTATAGTTTGAAGTCATGGCTCCCTTTTTTTACTCTT | |
| | | | CTTTTTTCTTTCTCAGAGAAGAGCAGGTAACAATGTGGGGTTAAAGGTGAGCA | |
| | | | GGTGGGTTAGAGTAGTGGAGCTAAAGGCAGATAGAGGTCTTGATCCAATTAT | |
| | | | CCTCATTCCCTTCATTTGTCCAACCTTGCACATTCCTGTATGCAGGGCCTTTGT | |
| | | | AAGCTTCACCTTCTGTACAGGTGCCCAGCAACTGTCAAACCAGCCAGGGCTTA | |
| | | | ACATGAGCATGGGCATTCCCTATGGACTGGCTGGGACACACCCACATCCTTAG | |
| | | | ATTACACATTTTGCCTGTAACATAGATAAACTAATTACTAGGTATATAATTCTG | |
| | | | TTTCGTTTTGTTTTGCTTTGTTTTGTTTTTGGGACAGGGTCTCATTCTGTCAAAC | |
| | | | AAGCGGGAGTGCAGTGGCACAATCACCACTCACTGCAGCCTGGACTTTTCTGG | |
| | | | GCTCAGGTGATCTTCCCACCTCAGCCTACTGAGCAGCTGGGACTACAGGTGTG | |
| | | | TATCACCACTCCCAGCCAAAATATTTTTTTATAGAGACAGTGTTTCACCATGTT | |
| | | | GCCCAGTCTGGTATCGAACTTCTGGGCTCAAGCTATCTGCCTGCCTTGACCTCC | |
| | | | CAAAGTGCTGGGATTACAGGTGTGAGGCCCTGTACCCAGCTCAATCCTGTTTT | |
| | | | TATACTAGAAGAACATTCTCTATCTGGGTCTTCCAAACTTGATTCTTACTGGTT | |
| | | | AATTTGTCCTGTTCTTTTGCTGGTCCAAGAAAATATCCTGAAATCTTTATTTCT | |
| | | | TCTCCGTTTCCTCCTTGTCCTAGGACAGACTAGCCCTATCCCCTTCCTGAATTA | |
| | | | AGTCCGAATATAGTCAGTCTTTGAGTGTGGAATAGCTCCTAGCAGTCTATCAG | |
| | | | TCAACGGGTTCTCTTTGTGGTCACATTCTATGTTTATTCAGGAGACTACAGCAT | |
| | | | GGAAAGAAAATGGACTTTGGAGTCAGGTGAATCTTGATTCAAATCTTGGCAGT | |
| | | | GCCATTCATCAGCTCTGTGGCATTGAGCGCATCAGTGGACCACTCTCTGATCC | |
| | | | CCAATTGCCTTGTCTGTAAAATGAGATTAGACACCCCTGCTCAAGATTATGGT | |
| | | | AGGATTATATATAATGTGTGTAACACAGCTTTTCCAGTGCCTGGTACAAGGTA | |
| | | | AGTGTCCAATAAGAAGTTACAAATGTTCCTGAGCCACCCTACTGGGTCCCTCC | |
| | | | TCACATCCAATGCTGGACTCTTCATGCCCAAGGAAACATATAAGGCACAAAA | |
| | | | GTCAGTGGCACTCAGCTCAGCAGAAGAAGGCACAGGAAGGGGATGGGGGAG | |
| | | | TCCTGGCTCCCACTTTGTTCTTGGGGTCAGGCTTGTGGCCAGCCTGGAAAGAC | |
| | | | TGACATGAACCCTCCAGCATCTGAGGGTGCCAACCACCAAGAGCAGCACAGT | |
| | | | TCTTGTCTACAAGCCACTTGTAGCAGGTGTGAACATTTCATCTTTTCCTCTGGG | |
| | | | GTTTGCAACTCTCTCCCCAGTCTCGGTCACTGCCTTTGGCTGTACCACTTCCCT | |
| | | | TTTCTTCTCGCCTGCACCTCCCTCATCTTTCCTCTATGATGACATCGCCCTGGG | |
| | | | GAAGAGAAGCTGAGAGGAACTCCTCACTCAGCTAGCTTCAGGAGCATGACGT | |
| | | | CATCTCTACCATGGAAATTCCACTCACTCTCCTGTGCCCCCACATTTGTCCTAG | |
| | | | GCCTCAGAGTCCCTATAAAGAGAGATTCCCAAGTCAGTATCAGCACAGGACA | |
| | | | CAGCTGGGTTCTGAAGCTTCTGAGTTCTGCAGCCTCACCTCTGAGAAAACCTC | |
| | | | TTTTCCACCAATACCATGAAGCTCTGCGTGACTGTCCTGTCTCTCCTCATGCTA | |
| | | | GTAGCTGCCTTCTGCTCTCCAGCGCTCTCAGCACCAAGTAAGTCTACTTTTGCA | |
| | | | GCTGCTATTTCGAGTCAAGGTGTAGGCAGAGTCCTTTTTTCTAGTCATGGCTGG | |
| | | | CAAACAGTGGGATCTGGGGATGGGACAAAAGGCAGCTAGGAAGATTGCCATG | |
| | | | TAGTCTGCTGCTAAATGTAGAGTCTAGTAGATATTCAATAACATTCAAGTTCC | |
| | | | TATTTTCTTAAGAATTAGCAACCAGCAGAGGAAAACGATGGGCTGGAAGTCA | |
| | | | GACTGTTGAATTGGCTCTGCCTTTAATTATTTGTTCAAGCAAGCCCCTGTCCCT | |
| | | | CTCTGTGCCTTGGTTTCCCCATCTGTCATATGAAGGGAGTGCGATGTGTTCTGA | |
| | | | GACTGAATCCAGTTCCAATCTTCTAGATTTCTTTCTCGTTCTTCTCTGAAGATC | |
| | | | CACTATTCAAAATAAGACTCCTGCTCATGTTAGGTGGGAATGGATATTTGGGG | |
| | | | TTCTGGTAGCTCCACAGGGATGCTCAATGAAGATACAAAATTAGAAGTCAAA | |
| | | | ATAAACAGCTCCCACGGGCAGTGTTGATCTCACCCTGGCCTTTCCTTTCAGTG | |
| | | | GGCTCAGACCCTCCCACCGCCTGCTGCTTTTCTTACACCGCGAGGAAGCTTCCT | |
| | | | CGCAACTTTGTGGTAGATTACTATGAGACCAGCAGCCTCTGCTCCCAGCCAGC | |
| | | | TGTGGTGTGAGTATCAACCCCTGGGCTGCCCTGGGAGGCAAGGGTGAGGGCT | |
| | | | GGATTTTAAAAGAGGGCCTGTTTTGGGGAGGGGGTGATTGAGCATTGGGGAG | |
| | | | GCAGCTCCCAGGGCTGAAGCCTTCCCTGAGAGCAGTGAGGACACAGGTCATG | |
| | | | AACTCACTTTTCAAGTGCTGAAGGCGGCAGAGTGGGAGCCGAGAGAGAAGGG | |
| | | | GGTTGCTGGGGAGGAAGTTATTCAGAGGACAGGGAAGCAGGGGAAGGCAGA | |
| | | | CAGGTCCCATGAGATATGGACCGATTCCTTAAACCGTGCTAGAAAGACATGTG | |
| | | | GAAAAGTCACTGCCAGGCTGGCAGGGAATGGGGAGATCTATTCATATTGATT | |
| | | | GCAATGCCCCTTGGTTCCTAATCTGGGCAACTCCTGGGGCCCACAGCTAAATC | |
| | | | CAGTGGGTGGAAGTTACAGGGAGTCTGCTTCCAGTGCTGCTCCAGGAAGGATC | |
| | | | CCATCCACCAGAGCTGCCCCACATGGACCATGGTCAGGCAGAGGAAGATGCA | |
| | | | AAGGATAAAGCCAGATGACCTCAAAGGTCTCATGAGATTCTAATCTGTCCGCT | |
| | | | CCTTGTTCTACAGATTCCAAACCAAAAGAAGCAAGCAAGTCTGTGCTGATCCC | |
| | | | AGTGAATCCTGGGTCCAGGAGTACGTGTATGACCTGGAACTGAACTGAGCTGC | |
| | | | TCAGAGACAGGAAGTCTTCAGGGAAGGTCACCTGAGCCCGGATGCTTCTCCAT | |
| | | | GAGACACATCTCCTCCATACTCAGGACTCCTCTCCGCAGTTCCTGTCCCTTCTC | |
| | | | TTAATTTAATCTTTTTTATGTGCCGTGTTATTGTATTAGGTGTCATTTCCATTAT | |
| | | | TTATATTAGTTTAGCCAAAGGATAAGTGTCCCCTATGGGGATGGTCCACTGTC | |
| | | | ACTGTTTCTCTGCTGTTGCAAATACATGGATAACACATTTGATTCTGTGTGTTT | |
| | | | TCATAATAAAACTTTAAAATAAAATGCAGACAGTTTCTTTGTGATTTTAATTTG | |
| | | | ATTTGGGGGTAAAAGGAATTGCCTGGTACCATTGGGAGGGACAAACTACAGT | |
| | | | TTCCCAAAACAGTAAGAATGAGCAATTGCTGAGTCCTTAATATGAGCTCTGGG | |
| | | | CTGAACACTCTTAATACACGATCTCACTGCGTACTTTCAACAAGGGTTTTAAC | |
| | | | ACCCTTATGAACTAGGGACTGTTGCACCGAGTTTCACAGTTAAGGAAACAGAG | |
| | | | GCACAGAGAGGTGAAGTGACTTGCTGAAGCTTGTAGGCCTGCTTGGGGAGTG | |
| | | | TCATGAAAGAATGCCCCAGAAAAGGAGGTTATCATTTCAACATCTTGTGGGGG | |
| | | | GAGTTGTAAAGAGGGGAAGCAGCACAGCAAAGGCGGAAGAAGAGTGAGAAG | |
| | | | AGTGACTTCATCATCTTATTATTATTATAACATTGGGAAAGTCATCTTGCCTTA | |
| | | | TTAGACCTCGGTGTTCACAAGTGTGAAATGGGGATAATATTAACCACTTCGAA | |
| | | | GGGCTGTTATAAGGTGATGTATAAAGCACCCATCATAGGACCAGGCACCTGG | |
| | | | AAGTTTCTCTCCTTCCCTCTCTGAGCTTCAGCCTTTTCACTTAAAATGGACCTA | |
| | | | GCAATTGTCAGGATCATCATGAGGATTAGATTAGTTAGTTGACAGATCAGCAC | |
| | | | TCTAAATCCCAATCTACTGTGAAGTGTTGTCATGAGGACAGCAGGATGGAGGT | |
| | | | TTTGTTTGGAAGTGTTGCAGGAGTAACCTAAAGCCACTTCTACTTAATTATCCT | |
| | | | TCAATCAGAACTGCCTATTCCATGGGTCAGACACAACACAACTCTGAAGTAGA | |
| | | | ATTACATGCACAATAAAGCCTGATCCATGGGACACAGACTCGTAGTCTACTAA | |
| | | | AGCTAAAAGGAAAGTTTAGACCATCTGGTCCAACCCTCTCATTTTATGGAAAC | |
| | | | TGAGGGCCAAGGCCTGGGAAGTAACTTGCCACGTGCCACTCAGCGAGTGCAT | |
| | | | AGCAGAGCAGGAGCCTGGCCAATGCCAGCTCCCAGGCTCGGGCCCTTTCCTGC | |
| | | | ACACCTTCCCTAGTGACGCATCTCTGTGGTCCTCAAAAGGGAGACCGGTTGAG | |
| | | | TAAGAACAAGGCGGAGACTATCTTGAAAATCATTTGAGTGTTCTTAGTCCTAT | |
| | | | GCAAAAGCAGAATTGGGGCCCTGCACTTCTCTGGGGTCAACAGACACAATTCC | |
| | | | CTTCCTTGTCCTGCCCTTAGTCACCCTCATCATTCCAGAGCCCCAGTGGAAACT | |
| | | | TCTTTCAGATTCTGCGGGGACTGAGAGAAGGAGGTTAGGGGGCAGAGCTGAT | |
| | | | CCAAAACAGGTGTAGGAAGAGGCTAGCAGCAAAGAATGTGCCTGGCCATAGT | |
| | | | TTCTGCTTTCATTTGCCTAGATCTGAGCTGTCTAATATGTTAGCCACTGGCCAA | |
| | | | AGTGGCTATTTACGTTTTCATTACATTTAATTAAAATGAAATAAAATTTAAAAT | |
| | | | TCAGTTCCTGAGTCACACTAGTCACATTTCAAGTGCTCAGTAGCTCCATGTGG | |
| | | | CTAGCTCCCATGTTAGACAGCACGGATTACGGGCTTTCTGTCCATCATTGCAG | |
| | | | AAAGTTCTGTTGGAGAGCAGTCTCCTAGAATGTTCTCAGAGCCTAAAGAAAGT | |
| | | | GAAGGGAAGGGGAGTTGGGATGGCCTGTAAGATTAGAGTGTGGATITTGAGA | |
| | | | AAGATATGCAATTCTATTCTGAGGCGACGGGATGGGAGAGAGAATGGATGAC | |
| | | | TCCAGGCCAGAGACAAGGTATAGATCACTACGGTAGCTGAATAATGGCCCCC | |
| | | | GAGGTGCACACATCCTATTCCTCAGAACCTGTGAATGTCACTTTTATAGCAAA | |
| | | | ATGGACTTTGGAGATATGATCAAGTTAAGAATCTTGAGATGAGTGATTACCTT | |
| | | | GAATTATTTGGTCAGGCCCTAAATGCAGYMTAAGTGTCCTTATAAGGGGGAG | |
| | | | GAAGAGAAGAATTTGGTAATAGAGGAGAAGATAGCAATGTGACAACAGAAG | |
| | | | CAAGATGCTATGCTGCTGGCTTGGAAG | |
| 55 | CXCL9 | >NP_002407.1 C-X- | SEQ ID NO: 105 | |
| | | C motif chemokine 9 | >NM_002416.2 Homo sapiens C-X-C motif chemokine ligand 9 (CXCL9), mRNA | |
| | | precursor [Homo | AAAATGTGTTCTCTAAAGAATTTCTCAGGCTCAAAATCCAATACAGGAGTGAC | |
| | | sapiens] | TTGGAACTCCATTCTATCACTATGAAGAAAAGTGGTGTTCTTTTCCTCTTGGGC | |
| | | MKKSGVLFLLGIIL | ATCATCTTGCTGGTTCTGATTGGAGTGCAAGGAACCCCAGTAGTGAGAAAGGG | |
| | | LVLIGVQGTPVVR | TCGCTGTTCCTGCATCAGCACCAACCAAGGGACTATCCACCTACAATCCTTGA | |
| | | KGRCSCISTNQGTI | AAGACCTTAAACAATTTGCCCCAAGCCCTTCCTGCGAGAAAATTGAAATCATT | |
| | | HLQSLKDLKQFAP | GCTACACTGAAGAATGGAGTTCAAACATGTCTAAACCCAGATTCAGCAGATGT | |
| | | SPSCEKIEIIATLKN | GAAGGAACTGATTAAAAAGTGGGAGAAACAGGTCAGCCAAAAGAAAAAGCA | |
| | | GVQTCLNPDSAD | AAAGAATGGGAAAAAACATCAAAAAAAGAAAGTTCTGAAAGTTCGAAAATCT | |
| | | VKELIKKWEKQVS | CAACGTTCTCGTCAAAAGAAGACTACATAAGAGACCACTTCACCAATAAGTAT | |
| | | QKKKQKNGKKHQ | TCTGTGTTAAAAATGTTCTATTTTAATTATACCGCTATCATTCCAAAGGAGGAT | |
| | | KKKVLKVRKSQR | GGCATATAATACAAAGGCTTATTAATTTGACTAGAAAATTTAAAACATTACTC | |
| | | SRQKKTT | TGAAATTGTAACTAAAGTTAGAAAGTTGATITTAAGAATCCAAACGTTAAGAA | |
| | | | TTGTTAAAGGCTATGATTGTCTTTGTTCTTCTACCACCCACCAGTTGAATTTCA | |
| | | | TCATGCTTAAGGCCATGATTTTAGCAATACCCATGTCTACACAGATGTTCACC | |
| | | | CAACCACATCCCACTCACAACAGCTGCCTGGAAGAGCAGCCCTAGGCTTCCAC | |
| | | | GTACTGCAGCCTCCAGAGAGTATCTGAGGCACATGTCAGCAAGTCCTAAGCCT | |
| | | | GTTAGCATGCTGGTGAGCCAAGCAGTTTGAAATTGAGCTGGACCTCACCAAGC | |
| | | | TGCTGTGGCCATCAACCTCTGTATTTGAATCAGCCTACAGGCCTCACACACAA | |
| | | | TGTGTCTGAGAGATTCATGCTGATTGTTATTGGGTATCACCACTGGAGATCAC | |
| | | | CAGTGTGTGGCTTTCAGAGCCTCCTTTCTGGCTTTGGAAGCCATGTGATTCCAT | |
| | | | CTTGCCCGCTCAGGCTGACCACTTTATTTCTTTTTGTTCCCCTTTGCTTCATTCA | |
| | | | AGTCAGCTCTTCTCCATCCTACCACAATGCAGTGCCTTTCTTCTCTCCAGTGCA | |
| | | | CCTGTCATATGCTCTGATTTATCTGAGTCAACTCCTTTCTCATCTTGTCCCCAA | |
| | | | CACCCCACAGAAGTGCTTTCTTCTCCCAATTCATCCTCACTCAGTCCAGCTTAG | |
| | | | TTCAAGTCCTGCCTCTTAAATAAACCTTTTTGGACACACAAATTATCTTAAAAC | |
| | | | TCCTGTTTCACTTGGTTCAGTACCACATGGGTGAACACTCAATGGTTAACTAAT | |
| | | | TCTTGGGTGTTTATCCTATCTCTCCAACCAGATTGTCAGCTCCTTGAGGGCAAG | |
| | | | AGCCACAGTATATTTCCCTGTTTCTTCCACAGTGCCTAATAATACTGTGGAACT | |
| | | | AGGTTTTAATAATTTTTTAATTGATGTTGTTATGGGCAGGATGGCAACCAGAC | |
| | | | CATTGTCTCAGAGCAGGTGCTGGCTCTTTCCTGGCTACTCCATGTTGGCTAGCC | |
| | | | TCTGGTAACCTCTTACTTATTATCTTCAGGACACTCACTACAGGGACCAGGGA | |
| | | | TGATGCAACATCCTTGTCTTTTTATGACAGGATGTTTGCTCAGCTTCTCCAACA | |
| | | | ATAAGAAGCACGTGGTAAAACACTTGCGGATATTCTGGACTGTTTTTAAAAAA | |
| | | | TATACAGTTTACCGAAAATCATATAATCTTACAATGAAAAGGACTTTATAGAT | |
| | | | CAGCCAGTGACCAACCTTTTCCCAACCATACAAAAATTCCTTTTCCCGAAGGA | |
| | | | AAAGGGCTTTCTCAATAAGCCTCAGCTTTCTAAGATCTAACAAGATAGCCACC | |
| | | | GAGATCCTTATCGAAACTCATTTTAGGCAAATATGAGTTTTATTGTCCGTTTAC | |
| | | | TTGTTTCAGAGTTTGTATTGTGATTATCAATTACCACACCATCTCCCATGAAGA | |
| | | | AAGGGAACGGTGAAGTACTAAGCGCTAGAGGAAGCAGCCAAGTCGGTTAGTG | |
| | | | GAAGCATGATTGGTGCCCAGTTAGCCTCTGCAGGATGTGGAAACCTCCTTCCA | |
| | | | GGGGAGGTTCAGTGAATTGTGTAGGAGAGGTTGTCTGTGGCCAGAATTTAAAC | |
| | | | CTATACTCACTTTCCCAAATTGAATCACTGCTCACACTGCTGATGATTTAGAGT | |
| | | | GCTGTCCGGTGGAGATCCCACCCGAACGTCTTATCTAATCATGAAACTCCCTA | |
| | | | GTTCCTTCATGTAACTTCCCTGAAAAATCTAAGTGITTCATAAATTTGAGAGTC | |
| | | | TGTGACCCACTTACCTTGCATCTCACAGGTAGACAGTATATAACTAACAACCA | |
| | | | AAGACTACATATTGTCACTGACACACACGTTATAATCATTTATCATATATATA | |
| | | | CATACATGCATACACTCTCAAAGCAAATAATTTTTCACTTCAAAACAGTATTG | |
| | | | ACTTGTATACCTTGTAATTTGAAATATTTTCTTTGTTAAAATAGAATGGTATCA | |
| | | | ATAAATAGACCATTAATCAGAAAACAGATCTTGATTTTTTTTCTCTTGAATGTA | |
| | | | CCCTTCAACTGTTGAATGTTTAATAGTAAATCTTATATGTCCTTATTTACTTTTT | |
| | | | AGCTTTCTCTCAAATAAAGTGTAACACTAGTTGAGATAAAAAAAAAAAAAAA | |
| | | | AA | |
| 56 | ICOS | >NP_036224.1 | SEQ ID NO: 106 | SEQ ID NO: |
| | | inducible T-cell | >NG_011586.1 Homo sapiens inducible T cell costimulator (ICOS), RefSeqGene | 135 |
| | | costimulator | (LRG_65) on chromosome | NG_011586.1 |
| | | precursor [Homo | 2AATATCAATCATACCATTTGGCAGTCACATCTCATGCTGATTTTTTTCACCCC | |
| | | sapiens] | TCATGGAAATATATAATCAGTTTTCTGGGCTTTATGGATTAATTAATAACTTTA | |
| | | MKSGLWYFFLFCL | AATGAGAAAATATTTGGCTGTCAAAATGGAATATACTTTTGTTTTATGATTAG | |
| | | RIKVLTGEINGSA | AGATGGGCAAGTGATGAGAGATGAATCAGGATGATGAATTATATATTTATTCA | |
| | | NYEMFTHNGGVQ | ATTAATTTCAATGTGAAATACAAACATGITTTGATAATTTTCTGCTCTCACATT | |
| | | ILCKYPDIVQQFK | ATGATTCAGAGTGAAAAATTGTCATTGGTGAGCATTCCCAAGGTCATATGGTT | |
| | | MQLLKGGQILCDL | CCTCCAAAAGTCAAGGATTCTTCAAACACTTGTTCATTCAATAAATAGTTGTG | |
| | | TKTKGSGNTVSIK | GAGCATTGCAAACATACTTGCAAAGAAGGCATCTTTAGGCTGAGGCGGGCAG | |
| | | SLKFCHSQLSNNS | ATCACAAGGTCAAGAGATCGAGACTATCCTGGCCAACATGGTGAAACCCCGT | |
| | | VSFFLYNLDHSHA | CTCTACTAAAAATACAGAAATTAGCTGGGCATGATGGCATGCACCTGTAGCCC | |
| | | NYYFCNLSIFDPPP | CAGCTACTCGGGAGGCTGAGGCAGGAGAATCGCTTGAACTCGGGAGGTGGAG | |
| | | FKVTLTGGYLHIY | GTTGCAGCGAGCTGAGAGGTTGCCACCGCAGTCCAGCGTGGTGACAGAGTGA | |
| | | ESQLCCQLKFWLP | GACTCCGTCTCAAACAAACAGTAACAAAAAAACCCGAAGACATCTTCAGTTCT | |
| | | IGCAAFVVVCILG | TGTTCTGATGTTTCTCACAGTTTAGTAGAAGCTGTTGGAAAGCAAACAGGCCA | |
| | | CILICWLTKKKYS | TTACAGTGGTATAATTGTTACAATTGTTACAGGTAGCTAGACAAGCATGAGCT | |
| | | SSVHDPNGEYMF | GGGCAGGAGAGGGCTCTTCCCCCACTCATAGGAATGCTGGGTGATGGTTTGGC | |
| | | MRAVNTAKKSRL | AATTATCACATTGCCTCTCTAAAAGTGATAAATTGGCAGCTGGTGCCAGGGAG | |
| | | TDVTL | AGGTCATTTCCTGATGGTCCACACCTGTTGCACTAAAGTGTTAATTGAATGCG | |
| | | | GGTACCAGGTAGAAGCAACTTCCTGGGCCTGCGCATTAAGAGACAAAATGGC | |
| | | | CTCTGGAGGCACACCACCAGAAAAGGGAAGAAAGCCTCAGATGAGCACACGT | |
| | | | ACAGTTTCCTAAACACACTGCATGTGCTCACCTCCTAAGGGTAAGGAGGGCAC | |
| | | | TGCACATGCGGGCAGCCCACCCTAAGGGAAGAATCCTGTGAAAGAGGCCAGC | |
| | | | CTATAAAGTCCTAAAATCAAGGTTAAACACCACACTTGACCTTGGGCCTCTTC | |
| | | | CAAGCACATGCTCCTTTTTTCTGTTCTAAAGCTTTTTAAAATAAACTTCTATTC | |
| | | | CTGCTCTGAAACTTGCCTCGGTCTGTATTTCTGCCTTATGCTTCTCAGTCAAAT | |
| | | | TCTTTGTTCTGAAGAGGCAAGAAATGAGGTTGCTGCAGACCTGTACAGATTTT | |
| | | | CTGCTGGTAACTAAGATACCTTCCACCGATAACATAATGATTTCTAGGATGGG | |
| | | | GGACACAGAGGAGCATTGGGAGGTTGCAGAAAAGCCACTAAATGGACACTAG | |
| | | | TTATGGGGGTGGTTGGGAGTGAATGACATTGGTTTGATGTCTTTGATGAATCG | |
| | | | ATGGGTATGGAAGCAAAGGCCTGAAGAATGGTGGAATATCTGCCAAAGAAGG | |
| | | | TAGGCTAGAGTGGTGAGGAGGGGAAGCACAGACAAAATGATGGCTCCTGGCA | |
| | | | GTAGAAGGAAGAGTTAGTTTGGGGAATTTGGAGAACTTCATGTCATTCAACGT | |
| | | | GATGGAAACATTTGGTACAGAAGGAAGAAAGAACAAATGTGGCAAGAGATG | |
| | | | AGGCCGAACTGGGGAAGGAGTTGCCCTTCATAAGGAATCTTGAAGGGGTGTG | |
| | | | GGCCTTACCTAAGAGAGGAAGCCAGTGGAGGATCCTGGGTGGGGCGGGGACA | |
| | | | CAGATGAATTCTATTTACATTTTACAAAGATTACTCTGGTTGTAGCATGAAGA | |
| | | | CAGACCAAAATGGGACAAGGGTGGGAATAGAAATGACAGAGGCCTGATCTAA | |
| | | | GTCAGTGACCTTGGCAAGCGAGGAGGAAAAAATACTTGAGAGACAGTTAGGA | |
| | | | AGTAGAGTAATTGGGGTTTATCGGTGAATCAGCAGGAAAAGGGAGAAGGCTG | |
| | | | GTTGGTGGTACCACTCAACAGTATAGGCAAGGTAGGAGTTGAAGCAATTGTG | |
| | | | ATTAGGAAGGACCTCTCACTTTTGATGCTTGTCCCTTTCAGAATGGCAAACCA | |
| | | | AGCTTAGGTGAAGCCTAGATATTTGGGGGTTACTTTCTAACCCTATTGCTTGG | |
| | | | ACTCAACCATGCTGGTGTCTGAGCTCCTCTACTCAGACAAATGTTGCTTTCAGT | |
| | | | AAGTTGCAAGCAAAGCAAAAGAAAGACATTCACCTACGGCCCTTCAGAAAAG | |
| | | | CAAGCATGCTGCATGATGAACTCTGGTGCCCAGGAGGCAAGAAATAAAAAAC | |
| | | | ACTTTTAATGTAACCAACTTTCCATGCCCCACCCTCACCCCAGGCGTCTGCATA | |
| | | | CACAAATCAAAACCATCTTAAGTCATGGAATGAAAAAAAATTAAAAGCAAAC | |
| | | | TAAAAGCACTTCCTGTGAGGTGAATGGAAATTTTTGCCCAGTTAATTTTCCAG | |
| | | | GCACGTGAAAGCAGTTCTGAATATTTTAAACCACAAGTACTTGTATTCTTGAC | |
| | | | CAAGATTTTTGAGAAGGCGAGGCATGGGAAGGATCATTGAAACAGGACTTTG | |
| | | | ACATAAAACATTGTTTACATAGAAGTGAGCTCTTCTCCACTATCGGATCAATT | |
| | | | TACCACCCAGTGTCTGACAAACTGAGAAGCGAGAGGGTTGGGAAAAAGCAAA | |
| | | | GAGATACATATCAAACAAAACCACACATATCCAGTTGAACTAAAGCTGAATTT | |
| | | | AACCATAACTTGTGCAACTTTTCTCCCCTTGAGGTAGAAACATTACACCTCCA | |
| | | | ATTGACCAGGACATCTTTTTTTGCATTCAAATTATCTTGGAATTTCCTCTAGGA | |
| | | | CCTAAGTATCCTTCCACCATGAAAATAGGAGACCAAAAAAAGTAATGCTTCCA | |
| | | | GTAATTTCTAGCCAAATAAATGAGGATAAGTGTGTGAGTGTGGTGGGGTAGTT | |
| | | | AAGAGTTTGGAATCCAGAGTTTCACTGTCTGGATTTGAACAGGCTCCGTTGCT | |
| | | | CATCAGCTCTGTGACTCCGGGCAAATGACTGAATCTCTCTAACTCTCAGTTTTC | |
| | | | TCATCTATGAAATGAGAATAAGTAACAGACTCTCCCTCGCTGAGTTGTTTGAG | |
| | | | AACGGGATCAGACAATGCACGTGGAGCCCCGTGTCTGGCACACACTATGTCCT | |
| | | | CATTGCATATTTATTATTAGGTTTCTGCTTTGTATTTTTCTCTGTGCAACAGCCC | |
| | | | TGAGCTCTAGAACTTATGACAGCTTTTCTCAAGTGAAGTTAAAGTTCCCCATA | |
| | | | CTCAGTTCCAGAGCTAGGCCTCACCTAGGTTGGAAGAAGTCAATTGTTCTCCA | |
| | | | CTGCCTGCCTGTGTGCTGGGAAAGGACTCTAGCACAGTGCCGGTGGGGCTGGA | |
| | | | AGCCTTTCCTCATGATGCTACGGGGAGTACAGTGAAAAGGGAGGAGATTGGG | |
| | | | GTAGCAAGGTCCCAGGGGTCCTGGCAGCCCAGGCTCTACTTCTCAGGCTAGCT | |
| | | | CAGTCCCTCAAACAGCTTCCTTCACACCCACTGCTGAGACCCAGGAGCCCTAC | |
| | | | AGAAGGTAGCTAGGGGAAGCTTCAGCAGCTAGGGGGATCCCTGCAGGATCAG | |
| | | | CCTCCTCCTTACCTTGCAGACCCCTTGGCTGTGGTCTGGCATTCCTAGTGGCTG | |
| | | | GCCTAAAGCCCCAAACCTGGAATCTGTTTCAGAGCCTGGACCTTTTGGACTCA | |
| | | | CACCTTGGTTTCCCTGGCCAGCTGATTTCAGCTTCTCATCTAAATGTCATTTCT | |
| | | | GCAGGCTAAGGGAAGTCCAGCCTGGATCTAGCATCTTGGAAGCGCGTTCAGA | |
| | | | CTTCTGGATGGAGGGATCAGCAGGCCTTGGTATTGGTGGTTCTACCTGCTTCG | |
| | | | GTTAAGAGTGAGGACTCTGGAGCCAGACTCTCTAAATCATAGCACTGACACTT | |
| | | | ACTATATGACTTGGGCCAGGTTCGTTACCTGTCTGTGCCTCAGTTTTCCTCATC | |
| | | | TGTAAAATGGGAGATTAGTGGTCACCATTGGGATGATTAAGAAGATTAAATG | |
| | | | AGTTAATGTATGTATAAACTATAGTTATAATGTAAATATATATACAAACAGTA | |
| | | | ACCATTTACATGCATATAAAACATAATATAAATGTAATATTAGCTATATTCAT | |
| | | | ATATAAAAATAGGTATATTTTATATATAAAATATATTACATTTAATTTTTTACA | |
| | | | TTTTGCATTAAGATATATTATTTCATATAAAACATTTAAGTACATATACATATA | |
| | | | ATTATCTATGTAATTATTATTGTTATTGTTGTTATTGTCTACTTTACCTCCCTGC | |
| | | | AGGGATGGGGAGACCACGGCAGGCTTTTAAACATTCTTGGTCTCACTGAGCCG | |
| | | | TAGGTGAGGAATCCCTGGTTCCTACAGACTCTGCTGTAATATGAGGAGCAGGG | |
| | | | TTTAAGTTAGTTTAAAACTGACCCCATCATTTGAAACATTGCAATCTACAATG | |
| | | | AATGCCACATAAATATCATTTCTCAGATTCCTATGATGCTCTTCTTTCAGATCT | |
| | | | TTTCACTTCAATTTCTATAATAATTTTGTTTGTTTCTTGTCCTATTTCAAAGGCT | |
| | | | TTCTTATCTCTGGAGCACCTAGCATAAGATAGAAATGTGTCAAAATATATGTT | |
| | | | TTATTCATCATGTGAGTATTTTTAGGTCCTGTTAACCCCCATAACTATTGATTC | |
| | | | AGAGAAGTAGGGTGGTTCTGAAAAATACAGGCATAATCTCTTTAACTTGTTTT | |
| | | | ATAGGAACCAGAATAAGGGTAATGTTTTCCTCTGTCTTCAAAATCATCAATAA | |
| | | | TCCATGCATTGTTTAACTCATGTCATAAGCAATAATGCCTTTCATATAGCCATT | |
| | | | GGCATCAAAGAAGAAACACCCCCTTGATTTGATGGTAAGCGTGACACTACAT | |
| | | | AAACTCCCAGAAAACCCACTTCCTTTCCAGCAAATAGAAAACAACCGAGAGC | |
| | | | CTGAATTCACTGTCAGCTTTGAACACTGAACGCGAGGACTGTTAACTGTTTCT | |
| | | | GGCAAACATGAAGTCAGGCCTCTGGTATTTCTTTCTCTTCTGCTTGCGCATTAA | |
| | | | AGTTTTAACAGGTAAGTGGTGTATTGAATATTTCTTATTAAGTTATAATTCAAG | |
| | | | TAAACATTAAGAAAAAGCAAAGGTAGAAAAATTACGCACCCAAAAGACAGTG | |
| | | | GTTTTGGTTTTTGAAAGTAGGAATAGTTTCAGGATATGGCCAAGGGCACCATG | |
| | | | TCACTCATGTCACTGTGGATGGCATTGAGTTTGCATTGCTGCCTTTGATACTGT | |
| | | | GTCTTGAGATGAAAAACATGTTTCATCAGACCATTAAATTTTTAAATTACCTG | |
| | | | CTTGGAGTTACCTGTCAGTGGATAAACTAGCAGAGTGGCTTACTGTTCTTAGA | |
| | | | GAAAGATGAAAGGCAAAATGACTTGTGTCAACAGAATGAAAATTAAATAGCT | |
| | | | CTGGCATTTGGACTGGGAATTCTTTCTGTGGTAGAAAATAAAATGGGAAGTCT | |
| | | | TTGAAAAGAAGGATTTTGATTTTAATCTTACCTGGATGATCTGTTATCTGAAAT | |
| | | | GTCCAGGGCACCATAGAGTTTTTATTGACACTAAGTATCAGAGTTCACCCTAA | |
| | | | AAAGCCAAGGAGGAAAGGATAATGTAAAAATACTTCCTAGCCCCTGTAGACC | |
| | | | TTACAAAAAAGATCATTCTGGAGTCCCTGATTCCTTCTCTTGAATGGCATATA | |
| | | | GCAGACTCTATATGGACAATTTAAATAATGCTACATACAATGTAAGTTAATAC | |
| | | | ACGTGTTTCTGCTGCTTTTTACTGTATATAATTTGTACTACTTTTGTTTTCTCTT | |
| | | | TCACACTGAGCCCTATTCTTGAGGTTGAAAAAAGGAAAGTTAAGTAATATTAA | |
| | | | ATAACTTAAATAATCAAATAAACTAAAAGTGAAGGCAGAACTTTTAGTAGGT | |
| | | | ATGATCCTATCTAGGGATACATTTTTTCAGTGGCAAAAAAATAGACCTTACTG | |
| | | | TTCTTACAAAAAATGAAAAAAAAATGAAATCAAATTGATCAAATGTTTCATCT | |
| | | | TGTGCTGGCTCAAAGTGAGTTAAGTGCAGTTAAGAGGATTGAGGAATGTAGA | |
| | | | ATGAGCACAAGGACAGTTTTGGGCTACTTGCTGGTTTATGCCTTTTCTAGAATC | |
| | | | TCATGAAGTCCTTTAGCGGCATGAATGACATGAGCAGCTTTGGAATTTATATT | |
| | | | CATTCTTCATTCATCCATTTACTCATGTAGCATTGAGCACCTGCCTGGTAGCAG | |
| | | | GTACTGTGCCAGATGGTGTATGTACAAGGATGAGCAAAACCAGGTCAACCCC | |
| | | | TGTTCTTTGGAAGGTAACTGCATGGTAGAGAGAGATATATTAATCAAATAATC | |
| | | | CCATTGATTAATGATAATTACAGGCAAAACTCAATTATATGAGGGAATGTAAC | |
| | | | AAGGAGCTTGACTAGACTGGGTGGGTGTGGAGGTGTAGCTGAGGAGCAGGGT | |
| | | | TCATTGAAGTCTTTCCTGTGCAAGTGGCACTTGAGCGGAGATTTCAAGGACAA | |
| | | | GGTAGCAATCAATGGGGAGAACGGTATGAAGGTAGGGAAGAACGTTCCATAC | |
| | | | CATGGAAGGAACAAGTGCAAAGAGCCTGTGGCAGGAAGGAATGAGGTAAGG | |
| | | | TTAGGGAACTAGAAGAAAGCAGACAGATGTGCCTGAGCACAAAGAGGGAAG | |
| | | | AGGCTGAGCTTGGTCCAAGATGACCAACTGGTCCAAGTTGGTCCAAGGTGAG | |
| | | | GCTGGAAAGGAAAGTAGAGGCCAGACCATTCAGACCATATAGTCTATGTTCC | |
| | | | AGATTTTGGATATTCTTCCAAGAACGTTGGTACCATTCGTGGAAGGGTTTGTTT | |
| | | | GTATTGATAAAAATCATTCAACTAGGCCAAACCAATTTGGGCAGTTCTGGTTG | |
| | | | TGGTATTATGCTCAGGAGAATTAAAGTAGCATCGGTGTCAATTCTGTGGAAGG | |
| | | | TGAGATCCAGCCTTAGGTAGGATTGTTTTAAGTTTCCAGATTAAGCTGACGAA | |
| | | | TAATTTGTATTCTGGAACAATATTCATCGTAGATTCTAAATGAATACAACCAA | |
| | | | TGTTCACTCTCATTATAAAGAAAGCCATGAGATGTGGCATGCTGTTTGGCATG | |
| | | | TGGCAAACCTGAGGCACGAGTGGCTAAATGGTAACAATAAATCTGCTAGCAA | |
| | | | TGATCCTATTTAAAATATCTCCACAATGCATGTATCCAGAAACTGCCATGTAA | |
| | | | AAGCTCTAAAGAAAAAGAGCCAAACTCAAGTAAACTAAGTACTTGCTTGTCCT | |
| | | | CTGTTGTATTTTCAATCCTCCCAATTTACATAGGCTTTCTCATGCCATGAAGAT | |
| | | | GTAGGCATTCTTACCTTTAGTCAGTGTTTGAAGAATTTCCAACCAGGAAAGGT | |
| | | | TAAGTGATTTGTTACACAGTAGTTGGTGGCAGAACCAAAGTTAGAACTCTAAT | |
| | | | GTGACTCCAACATCTACATTCTTTCTAAACTGTGTTGAAGTAGTGGAGGTGTA | |
| | | | ACATAAATCTAAAGGAACGTAAATCTCAATGATTCTGATCACCTTTGTTATTT | |
| | | | GGTTGTGAATTAAGAAGGATGGGAATCTTGCTGATTTCTAGTCTTTGATGGAT | |
| | | | CTCAATGTAGAATTTAACAAAGTCTTGTTTTTTTTTAATGAACTTGGAGCAATA | |
| | | | GGACACTTTTAATTATAAAAATATCTACGAAAAATGTGAATGTCTAACTACAT | |
| | | | TTTGAAGACCCTCTTTGTAGACCTTATAGGCAACAGGGACCATATGAGTGATG | |
| | | | GGACAAATTCTGGTTTTAGAAAGACTACTTTAAAAGCAGTGTGGATAATACAC | |
| | | | CAGATTGGGAAGGGCTGATGGCAGAGGGTGTCTAATAAACGTGCATGTATTA | |
| | | | TGATGTGTTCTGTGAGGAAGCTGGGAGGATAGCCCTCAAGGCCACTTTGAGCC | |
| | | | TTCCTTTGTCACTGAGGCATTCCATTAGTGACTCCTGAATTCCACTTGGGAAAA | |
| | | | GAGCCCCTCAGTGTCTCCAGCGTCTGAGAGAAGTCTGTGAAGATGACTGACCT | |
| | | | TGGAGCCCCTCTTAGCCTCTCCACGACTGAGTAAATGTCATCTCCTGACAGCA | |
| | | | CCCCCTTCCAGAGTGTTCTTGTTAGATTTAACAAAAATCCCAGTTGTCAATTTT | |
| | | | TTCTTTTGATAGATGAATTCAGGCCATTCCCAGTCAGCATAAGCCTCTACTTGA | |
| | | | TTTTATTTCTATGTTTATTTACTACTTATTTTTCTTTGTTGTATCCTCTTTTTCTC | |
| | | | TTTTATTTTTGTTGGTGTGATGAAATTTCTTTTGACTCTCCTCTTTGCCTCCGGT | |
| | | | TAATTTGGAAGGTCGACTACAGTTCTTAATTTACTAAGTAATTGACATGTTCA | |
| | | | AAGAATCTTAATTGAATGTCTACTTGTTTATCATTGTATGAAAATAATGAAAA | |
| | | | TGCCTTTGTCTGATGTGCCTAGTCCCCATCCCCTGCACTGATATGAGAACTTAA | |
| | | | GAATGTTCTTCCTTTCCCACTCTCACACTTCCCCCAGACAGTGCACCCTGCTCC | |
| | | | ATACGTGGGTTTTGTGGAAATAATTTAAAACTTACAGTTGCAGATCATTGCTA | |
| | | | GGTTTCCTCTTACAGTATTTCTCTTCTGTTTCAAGAATCCTTTCTTAACACTTAC | |
| | | | ATCACATACGTTCAATGACAACATCATTCTGGACATCTGCTCCTGTCTTGTGAT | |
| | | | CTCTGTTCTAATCTAATGATCAGAAGACTTCTCTTATTTTCGTTAAATACCCCC | |
| | | | AACTCTCTGAATTCCAGTACAGGTAAGGGTGTCTTCCTTTTACCTTGATGAGTG | |
| | | | ACTGATATCTTTCCTGGGAATAGGAGTCCTGGATGGCAGCCTTTCTTCTTAGTG | |
| | | | GTTGCAGATATTCCTCCACTGTCTGCTGGCTTCTAGGGTTGTGGGTGAAGAGTT | |
| | | | TGATGGTAGTCTAACTCTCATTCCTTTGTAAAAAAAAAAAAAACTCTAGAATT | |
| | | | TCATAATCTTGAAAGCTATTTACTTCAGTATTGGAATTCAGGAATTCCTCCAGG | |
| | | | ATATGTGTAACTCTCTCCCTCTGTTTCTCTTTCTGTGTGTTTCTATTAATTTTTTT | |
| | | | TTTGAGGCGGAGTCTCACTCTGTCGCCCAGGCTGGAGTGCAGTGGCGCAATCT | |
| | | | CGGCTTACTGCAACCTCCACCTCCCGGGTTGAAGCGATTCTCCTGCCTCAGCCT | |
| | | | CCCAAGTAGGTGAGACTACAGGCACCCGCCACCACGCCTGGCTAATTTTTTTG | |
| | | | TATTTTTCGTAGAGACGGGGTTTCACCGTGTTAGCCAGGATGGTCTCAATTTCC | |
| | | | TGACCTCGTGATCCGCCCACCTCCGCCTCCGCCTCCGCCTCCCAAAGTGCTGG | |
| | | | GATTACAGGCGTGAGCCACCGCGCCCCGCTGTTCTATTAATTTATTATTATTTT | |
| | | | TTTTAAGACTGGGTGAAAGTTTTCAATCTCTAGACTCAAGCTTTATTCTTCATT | |
| | | | TCAGTCATTTTTTTCCTAGTATTTGTTTAATCATACCTTATGCTGTTTTTCCCCA | |
| | | | CTCTTTCTAGAAATGTCTCTTATTCTTATGTAAGATCTTGAGTCCCTTATTTTTT | |
| | | | CATCATTTGTGAACAAAATGGCTTTCACAACTTGCTGTTGCTTGTTAACCACAA | |
| | | | CTCCTTCAGATGAAATGGATATTTCAGTTTTAAGTTACTAGTAGCATATTCCCA | |
| | | | TTTCTTGGTAGAAACAAAATGCAGGCATGTAGGGGGGACTGAAATTCTTATAT | |
| | | | TTACTCACTGTAAGATTCATAGCATTATTTGGCTTAAAAAAATTCATCCCAGTA | |
| | | | TTCACAGTTGTAAACAGCAAAATCTACAACATATACTGCTTCCCTACTCTAAG | |
| | | | AATATTGAGTTTTATATGCAAAATGACTTACTGGGCTTTGCAAATTTTTAAAA | |
| | | | ATGAGTTTTAAAATCATTTGTAGCTTGCAATAACTGAAGAAAAGATCACAATT | |
| | | | TCTCTGTAAATGGCATCTCAGTACTACTGCTGTTCTAATAAACTAGGTCTGTCA | |
| | | | AATGGCACTTTTTGGTCATTCAAAGTGAAAAAAACATGGCATTTGAAACCTTA | |
| | | | TAAAAATTCACAATTATATTTTGGAAAGTGGAACTTAAGTATGTAGGGGATAT | |
| | | | GTCACACAAATTGAAAGTGTTAGTCAAGAAATGATTTTTTTTTTCAATAGTTTG | |
| | | | GCCTGTAAGGTTGACCAAAACAGGTAAAGATTTGCAAGTTTAGAGAGTCAGA | |
| | | | AACTGGGGATATGAACTTCCTTCTGGTTCATGTATAACATAATGATGCTTGTAT | |
| | | | CTACTTTGCTTGAGGAAAAAAAAGAAACACGTTGTCCCCAAATAGGGTGATA | |
| | | | GACCAGGAGTTTCCATCCAGTTGACCACGAGTGTTGTTAGGATCTGTTTATTTG | |
| | | | AGTCATGTTGACACAGACACAGCACTTTCAATAAGCAATTAATGATCATTTAA | |
| | | | AGGTAACTTTTTTGAATTTGATAAATAGATTTTTTTTACAGCCTGTATTTGTGT | |
| | | | TCTTCTAGGACTTTGCAACAAAGAATCTGTACATGCAAAAACAACAATAACAA | |
| | | | CAACAAGCTGTCTACTGCACTCACAGTTAAAGTTCTGTTTACTTTGCACTATCT | |
| | | | AGGACTAATGCAAAAATCTCATAGATGTGCCTATTTGCACTTATTTAATTATG | |
| | | | ATGAAATTTACTTCTATATGTGAATCCATTTCTGTACATTTCTACAGGCAGATA | |
| | | | AGTGATGGGAAAATCATTAAGAATTTAGACTTCATCAGAGAAGAACCCAGGT | |
| | | | ACAATGACAGCCCAGCTTAATACATGATGGCTGATGAATGAAAATGTCTAGG | |
| | | | GAAATGAGTGTGTTATGTACAATGTAATTTCATATCTAAGTCACTTTAAAATA | |
| | | | ATTTTTTGATATATAATAACCCTTTCAATCACAGAACACATATGTGGCACCAA | |
| | | | TGTGAATACTACTTTAAGTATTAAGGTGATAATATATCCAATATACCTAGAAG | |
| | | | GAAAATAAATACCTGAAATTCATTTTCATATCTAGCTTAATGTTAAAAGCTTCT | |
| | | | TTTTGTTTTTTTATTTCGTAGCAAATCTCTTTAGCTCTGATGCTTTAGTTTCTGT | |
| | | | ACAATGAGAGGATTGGACTAGATTATTAGGTGAAGAGATTAAGTAAGTGTGT | |
| | | | GTACCCTGGTGTCAAATTATCTTATTCTTTTTTCTTTGTTTTTGTTGGACTGGGT | |
| | | | TAATTCGAAGACCTTGTCTTCAAGCTCTGAATTTCTTTCTTCTAGTTGTTCAATT | |
| | | | CTATTGCTGAGACTTTCCAGAGCATTTCACCTTTCTAAAATTGTGTCCAAAGTT | |
| | | | TCCTGAAATTTTTATTGTTTTTTCTTTAAGCTATCTATTTCCTTGAATATTTCTG | |
| | | | CCTTCACTTCTTGTATCATTTTTTGCATTTCCTTGCATTGGGCTTCACCTTTCTC | |
| | | | TGGTCCCTCCCCGATTAGCTTAATAACTAACCTCCTGAATTCTTTTTCAGGTAG | |
| | | | ATGAAGAATTTCTTCTTGGTTTGGATCCATTGCTGGTGAACTAGTGTGATTTTT | |
| | | | TGGGGGTGTTGAAGAGCCTTGTTTTGTCATATTACCAGAGTTGGTTTTCTGGTT | |
| | | | CCTTCTCATTTGGGTAGGTGCTGTCAGAGGGGAGGTCTAGGGCTGAAGGCTGT | |
| | | | TCTTCAGATTTTTTTTTTGTCCCATGGAGTGTTCCCTTGATATCATACTTTCCCC | |
| | | | TTTTCCTATGTATGTGGCTTCCAGTGAGCCAAACTGCAGTGATTGTTGTCTCTC | |
| | | | TTCTGGGTCTAGCCACCCAGCAAATCTACCCGGCTCTGGGCTGGTATTGGGGG | |
| | | | TTGTCTGCACAGATTCCTGTGATGTGAACCATCTATGGGTCTCTCAGCCATGG | |
| | | | ATACCAGTGCTGTTCTGGTGGAGGTGGCGAAGGGTGCAATGGACTCCATGAG | |
| | | | GGCCTTTAGCTTTGGTGGTTTAATGCTCTATTTTTGTGCTGGTTGGCCTCCTGC | |
| | | | CAGGAGGTGGCGCTTTCCAGAAAGCATCAGCTGTAATAGTATAGAGAGGGTC | |
| | | | TGGTGGTGGGTGGGGCCCTAGAACTCCCAGGATTATATGCCCTTTGTGTTCTG | |
| | | | CTAACCAGGGTGGGTAGGGAAGGGCAGGGCTAGGCATGTCTGAGCTCAGACT | |
| | | | CTCCTTGGGCAGGTCTTGCTGTGGCTGCTGTGGGGGATGTGGGTGAGATTCCC | |
| | | | AGGTCACTGGAGTTGTGTACCTAGGAGGATTATGGCTGCCTCTGCTGAGTCAT | |
| | | | GCAGGTTGTCAGGGAAGTGGGGGAAAACCGGCAGTCACAGGTCTCATCCAGA | |
| | | | TCCCATGCAAACGGAAGGGCTGGTCTCACTCTAATCATGCCTCCTCTAACCGC | |
| | | | CCCAAGTCTGTTTCCAGGCAAAGGGCAAGACTGGCTTGAAAACTTGCCTGAGG | |
| | | | CTTTCTGCCTCCCAGCTGTGAAAGAAAAGGGCTTTAGTTTTTCCCCTGCATGTG | |
| | | | AAATCTGCATGCTGGATTCACGGCCTCCCCCCAGTTCAGGCCAGGAGACTTCT | |
| | | | TGCCCTGTTCAAATAGTTACAAAGTTCAGCTAGAGAATTCCTTCTCCCTGTGG | |
| | | | AGTTTTATACCCTGCTCCTCTGGCCACCCTCCAGATGGATCCCTGTGGTGCCTG | |
| | | | GCAAGAATGGGCTGCTTGGGGACCCATTGAGCTCCCAGAGCCTTTCTGCAGCT | |
| | | | TCCTCTACCCCTGTATTTTGCTCCGTTTGGCTCTCTAACTTGACTCACCTCCAG | |
| | | | ATAAAGTCGGAAACTTCTCCACAAACAGACGCTCAGCTTCTCCGGTCAGAGTG | |
| | | | TGTGTTTGGGAGAGGAGGGTCTCCCTTTCCCGTGAGTCTTTTCTTTTTGACTGG | |
| | | | GAAATAAGTATCTCCCAATCTTCAGTAAATAGGATCTTTGCTCTCCTTTTCCCA | |
| | | | CTCCTCTTCTTGGCCCATTACAAAGATATTTACCAAACATGCTTAGCCTTTATA | |
| | | | TTCTTCTGTTAATATTAATTCTTTTATTTAATATGATTGAGCTTCCACTATGTTC | |
| | | | AAGGTCCTGGGATTTTTTAGGCAACTGTAATGCAGTCCTTATCTTTGTGGAGCT | |
| | | | CATGGTTAAATGAGGAAGACAGACAAAAATAATTATGATCCAAGATGCTATG | |
| | | | TGCTAAAATATACTGGCCTAAACAAAGTATTATTGGGAGCATAGAAATAAGA | |
| | | | GTCTTTGTCTGGTGGTCTTGGAAATAAATATCTCAAGAGAGAAGTTGACATTT | |
| | | | AAAATGGTTCTTGAAGAGTGAATGAGAATTTCTTCTCAGATTAAGGGGTAATC | |
| | | | AAATATTTTAGTTCATGGTAACAGAAATAATGAAATGTGAAGCATATTCTAGT | |
| | | | AGTAAAGAACAGAAGTGTGAAAGAAATGGCCAAAAAGTTCAGTGTGGCTTCA | |
| | | | ATAGATGGCCTATGAGATTGTGAAAGCATTTTGTGTCAAGCCAGATATATATA | |
| | | | TTTTAAAACACCTTATAAGCAATAGAACACTAATAGAGACTTTTAAACTGGGG | |
| | | | AGTGAAATGCTTTTCTGAAACATAATTTTGGGAGCAGCATGGAGGAGGGATG | |
| | | | GGGAGGAGAGAGATCAGTATTGAGAAGGCCAGTCAAGAGGATGCTTCCACAG | |
| | | | ATAAGGGAAGACATGAAAAAGGTGTGGATTGGCAATGGGAATGGAGAAAAA | |
| | | | GAATTGAATTTAAGAGATATTCATGAGCTGGAGTCAACTGGACTTGGTGACCA | |
| | | | ATTGGATGATGAAAATCAGAGAGAGGAAGAAATGAGGAAGACTCTGAGGTTC | |
| | | | CTAGGGTCAGACTAGTGACTCCCTGAAAAGACAGGGTTTGGATTTGGTGAATG | |
| | | | AATACAGTCACGTGCCACTTGACGATGGGAATACATTCTGAGAAATGTGTCCT | |
| | | | TAGTGATTTTGTTGTGTGAACATCATCGAGCGTACTGACGCAATGATGTCTAC | |
| | | | TAGATGGCAGAGCCTACTGCACTACCTAGGCTATGTGGAGTAGCCTATTGCTT | |
| | | | CTAGGCTACAAACCTGTACAGCATGTTACTGTACTGAATACTGCAGGCCATGG | |
| | | | TAGCACAATGGTAAATATTTGTGTAACTAAACACATAAAAGGGACATTAAAA | |
| | | | ATTTCTGTATTATAATCTTATGGGACCGCCACCATATATACAGTCCATCGTTGA | |
| | | | CCAACATGTCATTTATGCAGCGCATGACTGTACTGAGCTCAGTTTGGAAAGGC | |
| | | | TATACATGATGTGTTAGGGTAAAACCAGATAGTGATGTTGAAAGGAATTTGGA | |
| | | | AACAAGGAACTAGAAGTTTGGGCTGGAGAGAAAATGTGGGCTCCTGGGGGTA | |
| | | | GACATGAGAGCTGGAGCCATAGGTGCAAATGAGTTCAGGCAGTGAACACCCT | |
| | | | GTCCATGTGCCCAAGGAGTTTTGCACAGTAGCTGAGCAATGGCAAGCAATGTG | |
| | | | GGTATTCAGAGGTGTGGATAAAATCCTCTCTCCCTCCCACTCTGTGGAAGAGA | |
| | | | GTTGGGTTCATTTAATCTCATATGATTTCCCTAGTACAATAGTTTCAAATCAAA | |
| | | | GGCAATGAAGTAACTAACAAAATAACTTGTTTGTAAATTAATTGCTTTGTGAA | |
| | | | TATTGATACAAAGTTTTATAATACCACTGGGATTTTTTCATTTTTCATGAAGAA | |
| | | | ATCAGTTTTAACTAGGAGAAAATCAATCTTTCATTTTTCTTATTATACACACTT | |
| | | | AGAGAACCAAAGGGTTGTTTTGTACCCCCTATGGAGTTCCTTGGAGAGTAAAA | |
| | | | CAATGCACACATTGTGTGTGTGTTCATGAGCACTCTTTTGGCTTTTTCTCCTCA | |
| | | | CTTTTCTTCTTCCCATTTTTTTTTTTTTGCTTTCTTTTTTTTTTCTCCAAATGTTAG | |
| | | | GCATTTAGGTTAGATTGGGTTTATCTTGTCTATCCTCCCTCCCTTCCTTGCCTCA | |
| | | | ATTACTTCTTTAGATTATGGGTGCCTTTGAATAGCAATGTAAGTTTAGAAATG | |
| | | | ATGCATATATAAAAATCTACTAACCAGGGATCTAAAGGTTTTAGCTGGTAGAC | |
| | | | TCATAAGACATGACATTGCTTTTATGAAAGGTGAGAAAAGTGGTAGGAAGTA | |
| | | | CTTTTTTTTTTAATGCCAGAATTTCTTAGTAAAACATCAAATATAACTAAAACC | |
| | | | TAATATAAACATTGCAATTTTTAGACTTTTGCCTCTTGTGTCACATTCATGTGC | |
| | | | TGTGCTAAGCTAACACTTATCATTGTTTGATTACAGAAAATGTGTTTAATAAG | |
| | | | CACTATCAATGATTACAAACTCAGGACATCTTATCTTTCCATCACTCTGAGACC | |
| | | | TTTGTGGAGGTTTGTGGTTAGAAGTCTGAGTTAAAGAAAATAAAATTACATTT | |
| | | | CTGATATCTTGCACGCTCTGCACTGAGTGGGTTCTTTAAATGTTTCCTCCAGAG | |
| | | | GATTTTCAACCAGGAACAATACAACTGCTTGCAAAGAGTCCCAGCAGGATGG | |
| | | | GTCTGGGCATGAGCATCTTATACTGGTGAAATGAAGTAGTCCAGATATTAAAA | |
| | | | GCATCAACTTGCAAATGAACTCATCTGCACTATAGTGCAATTCTCAGGTGAAT | |
| | | | GACCTTTTCTTCAGAAATTTTCTTAATAGTCCCCCAAAAGAAGAAGAAAATGT | |
| | | | TCTTGTGCTTTATAAATTTTGAAAACCTAAAGAGATCACTTAATATAAAGATTT | |
| | | | TTATTTTACTCTTGAAAACATTAGTCTATTGATATCAACAGTAATATTATAAAA | |
| | | | ATTATTATTATTATTATTTTTTGAGACGGAGTCTTGCTCTGTTGCCAGGCTGGA | |
| | | | GTGCAGTGGTGCAATCTCGGCTCACTGCAACCTCTGACTCCCTGGTTCAAGCG | |
| | | | ATTCTCCTGCCTCAGCCTCCTGAGTAGCTGGGATTACAGGCACACACCACCAC | |
| | | | GCCCAGCTAATTTTTATATTATTAGTAGAGACGGAGTTTCACCATGTTGGCCA | |
| | | | GGATGGTCTCAATCTCTTGACCTCGTGATCCTCCTGCCTCGGCCTCCCAAAGTG | |
| | | | CTGGGATTACAGGGCGAGTCACTGCGCCCGGCCTATAAAAATTATTGTTAGCA | |
| | | | GCAGTTAACAGTCTTGAGCCTCTACTGGGAAGAATATGCCCTGTTAGACACAG | |
| | | | TATTCTATGTGATGAAGAAAATATAGTACCTGTTCTCCAGACTGATTATAATCT | |
| | | | AGGGTTTCCAATAAAAAGTAAACAGATATGGTAGCAAAACAAAACCTCATGT | |
| | | | TTAATATTCAGATTTTAAGACTCATCACCAGGATCCAAGACCACATATTCCTTT | |
| | | | TTGTGATTTTAGTAACATATTAAGTGCTAAAAGATATAAGATGATGTTTATAT | |
| | | | CACAAACCACTAACATGCTGTGTTTATAAAACATCTTGGTATATTAGAACAAC | |
| | | | CATTGGTCCAGGGGTTCTTGTCCTGGTCATATATTGGGCAAATGTAATGGCTG | |
| | | | CGCTTTGGGACATCCAAGTGAGCAACCAGGTTTGTTGCTCACTTGGATGGAAC | |
| | | | AAAGTTGTCCTGCCCTCCACAGACATATAAGAGCCAAGAGAATTATCAACTGT | |
| | | | CTAAATAAGTATTTATGAACCCAGTGGATATTTGAAGGTAAAGAAAGTTTTTC | |
| | | | TTTAAAGCTCAGACAGATAATTTCCATTAGAACATGACATCTCAGTTGGGGTA | |
| | | | GTGTCTTGTATGCATGATTGTGTATCTTCATACACTTGTTTCCAGAAGAAAAGC | |
| | | | AATAATATGGCATAGCTAACTCTGGGATCATGCCCCAGCTATGAGCTATGTAA | |
| | | | TCCTGGGCAAGATAGTCACCTTTTCTGGCTCTAGTTTAATTTGATAAATGCAAA | |
| | | | TGGAAAATGCAAAATGAAAGGTTTAACTTGTCATTTCTAGAAGCCCTTTTGGC | |
| | | | TCTTATATTATAAATCCTGTGCTTCATCTCCTCAGATTTAAAATGTCACCGTCT | |
| | | | ATATATTCATTTGTTCGCCCATCCAACCATGCTTTCATCCATCCAATAAATACT | |
| | | | GATTGCAGCTTACAACACACCAGGTATTATTGTAGATACTIGGGATGGATTTG | |
| | | | TGAATAAGACCAACAAGGTCCCTGGTTTTAAAGCTTAGAGTGGAGTAGCCAG | |
| | | | ACAATAAAGAAGAAAACAATCAGATAAACCAGATAATTTCAGACAGTGATAA | |
| | | | ATGCTTTATGTCAGTTAATGTACTAGATAATGACCTGGATATATTCTTTAGATT | |
| | | | GGGTGGCCAGTGGTCACTGAAGAAGTGTCATTACTCTTGAACGATGACAAAG | |
| | | | AAGCAGCCATGTGAAAATCAGAGAGAATGTTCTAGGTGGAGAAATGAGAAAT | |
| | | | TAAAAGCCCTGGGAACCTGCTTTGTGTGTTTGAGGAACAGAAAGAAGGGCAG | |
| | | | TGTGACTGTGGTGTAGTGTGTGACTGGTATAAAATGGGGAGAGGTAGGAGAG | |
| | | | GTCATGCAAGTCATGGGAAACCAGGGTAAGAACTTAGATTTTACTGTAAGTGG | |
| | | | AATGAGAAGCACCTGGAGGTTTTATAAGCAGAACAATGACTCTATTGGATTAT | |
| | | | GCATGTAAGAAGGTCATTCTGGTTTCTCTGTGGAGAATGGATTGTAAATGGGC | |
| | | | AGGAGTAAAAGCAGAAGCTCTTGTAGTCCCAGAGAGAGATGACAATGGTTTG | |
| | | | GACCAGTTGAAGCTGAGATGGAGGAGAAGTGGATAGATTCTGTTGGACATAA | |
| | | | AACAGAGGTGCTGGAAATTAGGCGTTCTTCCTGCTCACAGATTTTTAGCACTG | |
| | | | GTCCTGTAACACTCAGTTGTTAGTGACCACTGTCTAGCTTCCTGTGGTTGCCTG | |
| | | | CTTCTATTATACTTCTGTTGATTGTTGCTAGTGACACTTTGACTGTGGATTGAA | |
| | | | CCAAAAATCTTTCATGCACCCAGGAAGCTGTGTGACCTCGGGCAAATAATTTA | |
| | | | ATCTGTCTGGGCCTTCAGTTGTCTGTATGTACACAAAAGAGGTCACCAGATCT | |
| | | | GACTGTGTATGATGCTGAGAAGAGGTTGAATCATGTTACAGGAAACAAAAAG | |
| | | | TGTAAAGCTGAGGGTATTTATAGGATGCCTGAAACATGAAGTGTTGATGAAAT | |
| | | | TTATTAGCAATGGAAATGGTATGTTAGGAAGCATGAGGAGCATATCAAAGAT | |
| | | | AGTATGAGAGAGGATGTTTTGGAGAAGATTTTATATTAAAGGTGGTATGGGA | |
| | | | AGAAAAGGCAAACATGAGAAAAAGGAAGAAAGAACAAGAAGAGAGAGATTG | |
| | | | GTAAGGGGAACACTGACAATTTTTAACTACTGGTATGGCAACAGAATTGACCA | |
| | | | AATAGATTTGACACCTTACCAATCAGAGCAGACACTATCTATAAAAGTGGTGC | |
| | | | TATCTATCATACCAGCATTCCAGGCTGAGTATTAGACTTGGAGATAAGAACAT | |
| | | | CCAAGAAGGATGGAGAAAAGATCCTAGATGCTTTTGCAGTTATAAGAAACTA | |
| | | | ACGTATTCAGATCTTCCTTAAATTGTCTATCCATTAAATGCTTTAGTCATGGTT | |
| | | | ATAAGGGGCAGTCTCCTTTAATACTATTTTCAAATGTTACCAAAGGCTAAGAA | |
| | | | GAATTTGTAATTTGTCATTTGAGCATTGTCTTAGTCCATTTTCTGTTGCTATCAC | |
| | | | TGAATACCGGAGACTGGGTAATTTATAAAGAAAAGAAATGTATTTCTTACAGT | |
| | | | TCTGGAGGCTGGGGCCTAAGTCTAGGGGTCTAGGGGCCTAGGTCTAGGCTGA | |
| | | | GGCCTAGGGGCGAAATCTGGTGAGGGCCTTCTTTCTGGTGGGGACACTCATCA | |
| | | | GAGGCCCAAGGCAGTACAGAGGATTACATGATGAGGAGGTTCTTGAGAGACA | |
| | | | GCCAATCTGGCTGTTTATAACATACTAACTCTCATGATAATTAACCCACTCCTG | |
| | | | TGATAACCTATTAATCTGTTAACCCATTAACCCATGAATTGGATTAAGCTATTC | |
| | | | ATGGGGGTTCTGCCAATCATCTCTTAAAGGCCCCACCTCCCAATTCTATTACAC | |
| | | | TGGGGATTAATTTTCAATATGAGTGTCAGTCAGAGGGGGACAAACATTCAAGC | |
| | | | TATAGCAAGCATATCTGTTCTATATCTGCCAAAATAGTTATGAAATAATTAAT | |
| | | | TAAATGCTATAGTTAAAAACATTCGAACTGCAGACTTAGAGCTGTCAAGCGGT | |
| | | | TTTCCAGATGGAAAGTTTAAACACTGGATATGTGGAAACCTAAGTCAGCCTTC | |
| | | | TGATCACTACATTTCATAATAAATAGGCGATCCAGGTAGTTTTTAAGTTAAAG | |
| | | | ATAAAAAAATGAGTAATTAAGAGCGTCCAGGCCAGGTGCGGTGGCTCACGCT | |
| | | | TATAATCCCAGCACTTTGGGAGGCCAAGGTGGGCGGATCGTGAGGTAAAGAG | |
| | | | ATGGAGACCATCCTGGCCAACACGGTGAAACCCTGTCTCTACTAAAAATACAA | |
| | | | AAATTAGCTGAGCATGGTGGCATGTGACTGTAGTCCCAGCTACTTGGGAAGCT | |
| | | | GAGGCAGCAGAATCGCTTGAACCTAGGAGGCAGAGGTTGCAGTGAGCTGAGA | |
| | | | TTGCACCACTGCACTCTAGCCTGCTGGTGACAGAACAAGACTCCATCTCAAAA | |
| | | | AAAAAAAAAAGAGTGTCCAATAGAAATGACACTCTGAAATCTTCAGCTGATG | |
| | | | TCAGTGGCACACACTCTCAACATGCCATTCAAGAAAAATTGAGGCACGGCATT | |
| | | | CCTGGTCCTACCTAAATACTTTTGTGGGCAAGAAAGACAGTCTCCTTTATTTTA | |
| | | | ATTTATTCTGACTTTCTGGAATAGCCCATTATGATATTGAGACAGAAAGTGGT | |
| | | | TAACAGGAACATTGTTTTCCCTTTGGAAGACTTACCCAACAGCAAAGAATTAG | |
| | | | CTCTCTCAGTTGTTTGCAGTCTTATCTGGATATATTCTGATGCTGAATCCAGAG | |
| | | | AAAGCAGGTGTTAGAAATAACATTAAGATGTTTTTGTTTCACTTCTTCATTTCA | |
| | | | CGGATGAGGAGGCTGAATTCCACAGGGGTACCCTAGCCACAGAGCCTTGCTA | |
| | | | GTGGCAGAGCCAGAATTTGCCTCTGGTTTCCTTGGGATCCACTGCAATGCACT | |
| | | | TCCACCACACTGCACCGCCAACCATTCATGTCCTCTTGTGTTTGCCACAGTGCG | |
| | | | GGCACAATGGGGGAAAAATCTGCTGAGAAAATCATCTTCGGAAGTAGAAGAC | |
| | | | AATGCGTCTATGTCTGTGTATACATGGTGACCACAGACGCTTTGGCTCCAAGA | |
| | | | TCATTGTGGTTTACTTGCTGCCATTCTAAAGTCTCTGGGAAATTTTTTGTGAAC | |
| | | | ATTTCTAAAGGAACTGAGAACCCGTCCACTGATTTTATCTTCTGCAGCAGACG | |
| | | | GTAGAGACATACGCATCAAAGCAACAGTGGGGCAACATATTTTACTGTCAGA | |
| | | | CAAATAAAGCCCCAAGCAAAATACCCTACGTTTTAATCTAATGTAGACTGCAC | |
| | | | CTGTTCTCATGCCAACAGTTTTTTTTTCTTTGCCTCTGGATAATATTGTTCCTTC | |
| | | | CAGTTTTTAAAACGAAGCAAGTATTATTTTCCTTCTGTATGCTTTTAAACATAC | |
| | | | TTGTTATTATAAAACAAAACATACATATAGAAAAGTGAAAAAAATGTATAATT | |
| | | | TAATGAATAATGCTAAAGCATACTATCTTACCCTCATAATCACCACAGGTCAA | |
| | | | GAAATACAAGACTGTCAGCACCCCTATTTCCTTCTGATTCCAACTCTTTCTCCC | |
| | | | TCTTTAGACAAAACCATTAGGCTGAATTTTGTGATACTAATTCAGATTATATTA | |
| | | | ACTATTTCTATTTTTTAGTAATTATGGAAGAAATCAAAACATGTATACTTCACT | |
| | | | TAGAAAAGTTTCAAATTACATATACCTTCACTATCTTTCTAGACAATGTGCTCT | |
| | | | ATATTGACAGATGTTTCTTTTAGCACATTGGAGGTATATGTTATAATTCCACTG | |
| | | | TATCAGGGCTTTTATTGTTGATGTTGAAACAGTCTAATAGTTGCTCCTTGGAAG | |
| | | | GTAATCAGTCTCTTTTCTCTGGCTGCTTTTAGGATTTTCACTAAGATACATCTA | |
| | | | GGTGTGCATACTTTTAAAAAATATGCTTGTGAATTGTTGGGATTCTTGAATCTG | |
| | | | TGGAGTGTGACTGGGTTCTAACTAATTTTTCCAACCTATCTGCTGGTTCACCAA | |
| | | | TTTTCTCTTTAACTCTGTCTAATATATATTAAACTTATCTATTGAGTTTTAAATC | |
| | | | TCAGTGCATTTTTTTGGCCGATTCTTGTAGGTTCTTTTTCAAATTAGTTGACTTA | |
| | | | AATTTTCCTGTGTACTACAGATATTATTACAAGTTTTTCTCTTATTTGCTTAAAC | |
| | | | ATAATAAGCTTAATTACTTTATTTTGGGGACCTGATAATTCAGGTATATAAATC | |
| | | | ATGAGTTTGTTTTTGTAGTTTGTTGTTTCTGTTGTTCCCTTGTTTTCTTGTATGTC | |
| | | | TCATTATTTTTGTGATGTGTTTGCCATTGAATGTGAATATTATTTGTATGAAAT | |
| | | | TTTTTGAGGCCTAGGATGTAAACCTTTTCTTCTTGTAATAGTTTGCATTTTTACT | |
| | | | TTCAAAAGCTGAAGGGTAATAATAATGCAAATCTCCTTAAAGAAAATTCAAG | |
| | | | GATTGAGGTTTTCTAACCATTGAAGTCCAGAAGATTTTTCTGGACCATCTAATC | |
| | | | TGAATGAGGTCTGGTTTATTTCTTTACCTGTGCTCCAAGAATGTAACTCTITGG | |
| | | | GGTCCCAATTTATTGGGGAGAGGGTTTTCTGCTAGATCACTCACTCTGCAGAT | |
| | | | TCTGGGAGTTGAGACATAAAAACAAAAGTTTGAATTTGAAAGAACAGCAATG | |
| | | | CTTTCCGAGTAAAAGCAGCTTCCAAGCTTTGCCTGTCTCTCGGGGTTCTGGTTC | |
| | | | TCTCTTCAAATTTAGCCTGGTAATTTCTCCTCCTTTGTGTCATTCTTTAATGCTT | |
| | | | TTAATAATATTTTTGGACATTTGTTCAACATTTTTGGTTGTTTTGGCGTGAGAA | |
| | | | TTAGTTTGAATAATAGGGACTGCTATAACTGAAGACTAGAACACTTGTGCTTA | |
| | | | TTTTCACAATCAAATCACCCAAATTGAAGATATATAGGGAAAATATAAATTGA | |
| | | | AGCCCAGGGTCATTAAGAAAATAAGTAATTGTGTATCTTAAATAAAATTAAGG | |
| | | | CAACTAGTGTAAGTAGAGGTGTGACAAATAGCCCCTCCTGCATAATGCTAATC | |
| | | | CTGTTACAAATTTCAGAAAGCAAAATGGCAATACATAGTATTATAACTTTTAA | |
| | | | ACATTTTGACCAAATGTCATAATATTTACATAAAAGCAAAATGCTGAAAATAA | |
| | | | TCCCAATATCCAACGTTAGTATTTTAGTTITAACAAATTATGATAGATTAACAT | |
| | | | TTTGGCCAATAATATAGTCATTAAAAAGGATAATTATGATGACTAAAAATGAA | |
| | | | ATGTTTATGACATAAAATAACAATAGCAGAACACAAAATAACATGTTTGC TAT | |
| | | | AAATAATAAGTATGTTTTTGCAAGATAAAGAGCAAAAATATACAAATAGTATT | |
| | | | TCTATTTCACTGTAGAACTAGGTAATTTTITTCCTTAAAATTTTTTTACACTTAC | |
| | | | ATAGTTTTCTCAAAAACAAAGACCAAGATTTCAAAACTTAGGAAAAAACCAG | |
| | | | CATACTAATAATTTTCCCAAACAATTTCAGCATGAAAATTTGTCATATTAAAA | |
| | | | AGGAAAATAAAACAAAAAGAAAGAGAAGGCAATGAGCAGTTTGAATTTATA | |
| | | | GTCTAATAACTCAGAATCTGCAACCACCAGGCCAAGGTGATTTCAGTTTTTTC | |
| | | | CTTCTGTGATTCCCATGTAGCTTGCAAAAACAACCTTGGCTTCCTATAAGCATA | |
| | | | AATTTATTTCAGAAAATGAAAAAGTGGGGCTGGGCATGGTGGTTCACGTCTGT | |
| | | | AATCCTAGCACTTTAAGAGGCTGAGGCAGGAGGATTGCTTGAGGCCAGGAGT | |
| | | | TCAAGACCAGCCTGGGCAACATAGCAAGACCTGTCTCTACAAAATTTCTTTTT | |
| | | | CAAAGTTAGCCAGATATGGTGGTGTGTGCCTGTATTCCCAGCTTCTTGGGAGG | |
| | | | CTGAGGCAGGAGGGACCCTTGAGTCAAGGAAGTGGAGGCTACAACGAGCTAG | |
| | | | GATCATGCCACTGCACTGTAGCCTGGGGAACAGAGTAAACCCTTTTCTCTGAA | |
| | | | AAGAAAAGAAAAGAAAAGAAAGAGAGAGAAAAAGTAAACAAAAATCTGGG | |
| | | | GCTTTATCTTTATTATCAGGCCTTGATTCAGTTCTCAGTGGATGAGGTTTGGAG | |
| | | | AAAATACATTTCTGTCATTTGAAAATGATTAGTCTGTTCTTTTACTCTTTAGCA | |
| | | | GGAGGACATAATGTATGTGTGTATATATGTATGTATATGTATACAATTATTTGT | |
| | | | ATATATACACACACATAGTCTCTATATATTGTAATATACATATACATATAATA | |
| | | | CATATATTTTAATATATAATATGTAATATACATACATATATAAAATATATATAC | |
| | | | AATTATTCATTTTTAAACTACTTCGTACTTTTACAAAGTGAGCAATTTCTATCT | |
| | | | TTCCTTTTATCTGCTTTACTGATGTACATCAGTGATTATTTACTCATCAGAAGT | |
| | | | ACACATGGTTCTTGTCAGAGTAGTTTGATCAACAGGTAGACAATTCCTTCTCC | |
| | | | CCAAGATATACCTACTAATAAATGAAGTTTTTCCTAAACTTCAACTTTTCCCAT | |
| | | | ATCTCTCTGTTTAGCTAAGTGCTACTAATCCTTTAATTCCAATTTTAAAACTCA | |
| | | | CTTCCTCAAGGCGGCATTTTCTGATTCTATAATCAAAATTATAACCCACCTGAC | |
| | | | TCTACCACCTTCATTTAAATAGTACTCCATTCCTATCACAAATTATGTTGCATT | |
| | | | ACTACATATTTATTCCTTTACCATCTGCCTCCAATATGAAAGCTACCAAAGGA | |
| | | | CAGAGGCCATACCAGTTTGATTCATACACACTATGTAAATATATTATTTAATA | |
| | | | TTTAGTATATTCTATATTCACATATGACAAGCAAGTTTCTGGTTAGGATTTTTT | |
| | | | TTTCTCTAGGACATAGTTTTATCTTATGTTCTTCCTTGAGGGGTGGAGGGAGAA | |
| | | | TGTGAAGCTGTTTCATTTTGGTGCAACAGAGATGACTTTATGCATTGAATAAA | |
| | | | TTGCTTTTATGTTAAAATATAATTATTAGGGCAACATTATAAAATGTCACTTTT | |
| | | | GCTTTGTTTTCTTTCTTTTTATGCAGGAGAAATCAATGGTTCTGCCAATTATGA | |
| | | | GATGTTTATATTTCACAACGGAGGTGTACAAATTTTATGCAAATATCCTGACA | |
| | | | TTGTCCAGCAATTTAAAATGCAGTTGCTGAAAGGGGGGCAAATACTCTGCGAT | |
| | | | CTCACTAAGACAAAAGGAAGTGGAAACACAGTGTCCATTAAGAGTCTGAAAT | |
| | | | TCTGCCATTCTCAGTTATCCAACAACAGTGTCTCTTTTTTTCTATACAACTTGG | |
| | | | ACCATTCTCATGCCAACTATTACTTCTGCAACCTATCAATTTTTGATCCTCCTC | |
| | | | CTTTTAAAGTAACTCTTACAGGAGGATATTTGCATATTTATGGTAAGACATTG | |
| | | | CTTTCATCTTCCAAACTTAAGAGTATATATATGTTTTGACAACTTTTCTCACTA | |
| | | | ATAAAATCAATTAGACAAATAAATATCTTTTGTGTTGGAGTTCACTTAGATGC | |
| | | | AGTTGATGGTAATCATTTATAATGAAGATATACAGGTGATGATTTACTATTAA | |
| | | | TCATAATTAGTATTATTCAGCAATATGCAATGTAATCCCATAGACTGCTAAGT | |
| | | | CAGAAATAGATCATATTGCCTTTTAAACACATATGCTATTAAAAATCAGGAAA | |
| | | | AACATTAAGACAAATACTTAAACTTATGGCTTAATAATGGGTATTATCTCTAT | |
| | | | TCACCTAAGATTTAAGGTTTGGTTTTGCACTGTGTTTGTGGAATGAAGTATGAT | |
| | | | GATTGAAAATCATAGTTTTATAACATTACTTTTACTATTACCTCTTTTATAAAA | |
| | | | ATATAGGCAGAAAAGCCCTTTCTATTAACCACATTTGTAAATACATTATATGC | |
| | | | ATAATCTTTTAAGTTTTAGTTCCTGGATCCTTGCCATAATACTGAGATGATGTT | |
| | | | AGTCTAATAACTTGCCCACTTACTGGATTTCATGACTATAATAAAGAAAAGCT | |
| | | | TCATTTGATTAAATAGCTCTTTTAAATTTGGTAAGAGAACTTGTGGTTCAGCAG | |
| | | | AATTTTTCATTAACATACCTTTTTTTCCAATCCAGAATCACAACTTTGTTGCCA | |
| | | | GCTGAAGTTCTGGTTACCCATAGGATGTGCAGCCTTTGTTGTAGTCTGCATTTT | |
| | | | GGGATGCATACTTATTTGTTGGCTTACAAAAAAGGTAAGCGATTTCTATCTTTC | |
| | | | CTTGTATCTGCTTTACAGATGCACATCAGTGATTATTTCCTCATCAAAAGTACA | |
| | | | CATGGCTCTTGTCAGAGTAATTTGACCAACAGGGAGACAATTCCTTCCCCCCA | |
| | | | AGACATACCTACTAATTAAACTAATCACTTGGAACAGAAATTTATGTATTTGT | |
| | | | TTTATAGCCCACACATTCCAAAGAGGACTTGACTTGATGGCTTACAGAGAGTT | |
| | | | ACATTATTATTATTATTGTTCAAACTAGATGAGGAAATTGAGGTCAAGAGAAG | |
| | | | ACTAAAGGTCAGGAGGAAGGTTAAAACATCAAAATAGATGACTCTGATTTTT | |
| | | | AAAAAATTATTATGCATGGGCTGCGAATTTGAATCCAAGGTTCCTGAGAGGCA | |
| | | | GGGCAGAGATGAAACAATTAGTTATAAGTTTTGCTTAGCTGGTAAGTTAAAAA | |
| | | | CAGTCAGCAAGAAAGCCTTCTAAGGTGTCAATTGTAGCTGAGAAGGAAATTG | |
| | | | ACAACTTTTTTACATGGTAGGTTATTAGACTCCTTGCTTTTAAAGAAAACCACT | |
| | | | GAGGAGCTATTTAGAAAGATAAACAACAATGACAACAACAAAAATCTCCCTG | |
| | | | TGCTGGGGGATATTCTTTTTGGTCTCTTGCATTACAGGTTCTCAATCAAAGAAC | |
| | | | TGACACCACTCTGCATATTACGCGAGTATGCTAGTACTCAAGTATATCCACAG | |
| | | | TGTGGTCATTGGCCAGTGTATATCTGTGGGAGAGTGGAATTTGGAAAGAGACA | |
| | | | TAAAGAAGGAAGAGAATGAGATAGCGCCCATGGTCTAAGTCTGCTGGGAAAG | |
| | | | AAACAATAAAGGCAAAAACGAGGAAGAAAGTTCAGTAGAAAGCTCTTTTTAT | |
| | | | GTAAAGCCCTTTTCATATTGTTTTTCTACTTAGCCTAAAGCCAGGAACCTAAGT | |
| | | | CACATTATCATGTTTTTGTCACATACCACTTCAACAAAGAATAGAAAACAGTC | |
| | | | AAAAAACAAAGAGATGGAGAAGAAAAGATGACCAAGCATGTTTCTGGCTTTT | |
| | | | TCTTTCAGAAGTATTCATCCAGTGTGCACGACCCTAACGGTGAATACATGTTC | |
| | | | ATGAGAGCAGTGAACACAGCCAAAAAATCTAGACTCACAGGTATGACTCCAT | |
| | | | TTGGGGGTTTGGGAAGGGAAGAGGTTTCTTCACAGTAAGCCTGGAATGTTAAT | |
| | | | TTTTTTTTTTTTAATTTTAAAGGAAAGGAAAACATCTCCAAGGCCTAATTCTAG | |
| | | | AATTTTCTGTGAAAATCTGGATTTCTCACTTTTAACTAGATTTATATTTTCTGC | |
| | | | AGTATATTGAAAACAAACAAGCAATAGGTTTGTAAGCCACTATTGAAACTAA | |
| | | | ACACACAAACAGCTCATAAACCACCATAGTATCAAGGCAATTTTCCCATTGAT | |
| | | | GTTTCAAAAGTTGGGATGATTCTAAGTGTTTTTTGTTGAAGGGTTCATTCAGTC | |
| | | | ATTCAATGAGCGTTTATTGAGCATCAGTTAAGTGTGAGTTACTGAGTGAGGCA | |
| | | | CTCTGGGGAATTTGGACAGATATATGCTAATTTTGGACTGAGATTAGGAAGAG | |
| | | | AGATCTGTGCTGGTACTGCTAGAGGGAAGAAGGAGAAATTGATGAAGATATC | |
| | | | TGGTTATTTTGAGGTAAAGAGTGTGCAAGTTTAGAGAACTTGGATGGCTTTAG | |
| | | | AAGTCTCAGTAACATGGAAATTTGACCTTTTCTGTTAAGAGTCAGAAAACTGA | |
| | | | AAGTTGCCAAGTTTGAGGAGCACCCAGAAAGTTTGAAGCTTCCATTGTGGAAA | |
| | | | ATTCATGTCTTATGCCCTTGAGGTTCGAATGATGTGCGATGTGTAATGAGACA | |
| | | | GAAGCCACTGCATTTTTCCACCCCTCAGTGGCTACAGATTATCTAAAGGGGAC | |
| | | | ACACATAGCTTGCGTTGTCATGGAATTATAGCATGGGATGGGAGCTAAAAAG | |
| | | | GATCCTTGAGTTCACATTTCATGTTTTAGATGACAGAAATTGATGCTCAAAGT | |
| | | | GTTCAGTGACTCTTTTGGTTCCCTAGACAGTTAATACCTAGCTTTGACACTTGC | |
| | | | AAAGAGCTTATGTTTCATTACTTTGCTGTTTAAAAAATATATGATCTCTCAATC | |
| | | | TCTTTGCTTAATGTGTTCTGGCTGTTTGGATCCCCACAAGCAGAACCTGACATA | |
| | | | AGGACTTGGGTGCAAGTAGTTTATTTGGGAGGTGATCCCAGGAAGAAGGGGT | |
| | | | GAGGGGGCAGGGAGAGTGAGACAGCCAACAAGGAGAAGCAAATGAAGGGCG | |
| | | | CATTATTATCAAGGTCTCAGCTCCAGGCCACAGAGGCCTGACTTCATTGGACC | |
| | | | TCTGAGAAGTGTACAGATGCCTCCCAGAATCATTCTCTTGAAGGATGGCAACC | |
| | | | ACTGGCTCCATCCTCACTGATTTGGGGTTGCCTGCGGAGGGCATTAGCTTTCCC | |
| | | | TCATCTCTGAGCTGTGCCTCTACTTGGGATGAGCCTACTTGGGATGAGTCTTCA | |
| | | | GAGAAAGTCTGGAGGCAGAAAAGCAGTGAAATGCAGTGGGGCTGTTGTGCTA | |
| | | | GAACAGTCAGCCCATGCAGCGCTTTCTGCCCCAGCAGCAGCTGCAATCAGAG | |
| | | | GTGGACAGTTTCAAAGTGATGAGTTTAGATAATGTCTGTTATAGGAGTCGAGA | |
| | | | GCTAAACTTCTCTGGAAATGGGGAACTGGTGCTGGGTGTTGAAGCATAAAGAT | |
| | | | GAGTTTGCATGGTGTGTGTGTGAGTGTGTGTGTGTGTGTGTGTGTGCACGTGT | |
| | | | GTGTTTGTGTGTATGAAAGGCAATGGAGAGGGGAAAGCTTCTTGTAGGGAAC | |
| | | | TGGCACATGGAGAGCATTTAGATAATTTATGCTGAATTTTTGTTACAGATGTG | |
| | | | ACCCTATAATATGGAACTCTGGCACCCAGGCATGAAGCACGTTGGCCAGTTTT | |
| | | | CCTCAACTTGAAGTGCAAGATTCTCTTATTTCCGGGACCACGGAGAGTCTGAC | |
| | | | TTAACTACATACATCTTCTGCTGGTGTTTTGTTCAATCTGGAAGAATGACTGTA | |
| | | | TCAGTCAATGGGGATTTTAACAGACTGCCTTGGTACTGCCGAGTCCTCTCAAA | |
| | | | ACAAACACCCTCTTGCAACCAGCTTTGGAGAAAGCCCAGCTCCTGTGTGCTCA | |
| | | | CTGGGAGTGGAATCCCTGTCTCCACATCTGCTCCTAGCAGTGCATCAGCCAGT | |
| | | | AAAACAAACACATTTACAAGAAAAATGTTTTAAAGATGCCAGGGGTACTGAA | |
| | | | TCTGCAAAGCAAATGAGCAGCCAAGGACCAGCATCTGTCCGCATTTCACTATC | |
| | | | ATACTACCTCTTCTTTCTGTAGGGATGAGAATTCCTCTTTTAATCAGTCAAGGG | |
| | | | AGATGCTTCAAAGCTGGAGCTATTTTATTTCTGAGATGTTGATGTGAACTGTA | |
| | | | CATTAGTACATACTCAGTACTCTCCTTCAATTGCTGAACCCCAGTTGACCATTT | |
| | | | TACCAAGACTTTAGATGCTTTCTTGTGCCCTCAATTTTCTTTTTAAAAATACTT | |
| | | | CTACATGACTGCTTGACAGCCCAACAGCCACTCTCAATAGAGAGCTATGTCTT | |
| | | | ACATTCTTTCCTCTGCTGCTCAATAGTTTTATATATCTATGCATACATATATAC | |
| | | | ACACATATGTATATAAAATTCATAATGAATATATTTGCCTATATTCTCCCTACA | |
| | | | AGAATATTTTTGCTCCAGAAAGACATGTTCTTTTCTCAAATTCAGTTAAAATGG | |
| | | | TTTACTTTGTTCAAGTTAGTGGTAGGAAACATTGCCCGGAATTGAAAGCAAAT | |
| | | | TTATTTTATTATCCTATTTTCTACCATTATCTATGTTTTCATGGTGCTATTAATT | |
| | | | ACAAGTTTAGTTCTTTTTGTAGATCATATTAAAATTGCAAACAAAATCATCTTT | |
| | | | AATGGGCCAGCATTCTCATGGGGTAGAGCAGAATATTCATTTAGCCTGAAAGC | |
| | | | TGCAGTTACTATAGGTTGCTGTCAGACTATACCCATGGTGCCTCTGGGCTTGA | |
| | | | CAGGTCAAAATGGTCCCCATCAGCCTGGAGCAGCCCTCCAGACCTGGGTGGA | |
| | | | ATTCCAGGGTTGAGAGACTCCCCTGAGCCAGAGGCCACTAGGTATTCTTGCTC | |
| | | | CCAGAGGCTGAAGTCACCCTGGGAATCACAGTGGTCTACCTGCATTCATAATT | |
| | | | CCAGGATCTGTGAAGAGCACATATGTGTCAGGGCACAATTCCCTCTCATAAAA | |
| | | | ACCACACAGCCTGGAAATTGGCCCTGGCCCTTCAAGATAGCCTTCTTTAGAAT | |
| | | | ATGATTTGGCTAGAAAGATTCTTAAATATGTGGAATATGATTATTCTTAGCTG | |
| | | | GAATATTTTCTCTACTTCCTGTCTGCATGCCCAAGGCTTCTGAAGCAGCCAATG | |
| | | | TCGATGCAACAACATTTGTAACTTTAGGTAAACTGGGATTATGTTGTAGTTTA | |
| | | | ACATTTTGTAACTGTGTGCTTATAGTTTACAAGTGAGACCCGATATGTCATTAT | |
| | | | GCATACTTATATTATCTTAAGCATGTGTAATGCTGGATGTGTACAGTACAGTA | |
| | | | CTGAACTTGTAATTTGAATCTAGTATGGTGTTCTGTTTTCAGCTGACTTGGACA | |
| | | | ACCTGACTGGCTTTGCACAGGTGTTCCCTGAGTTGTTTGCAGGTTTCTGTGTGT | |
| | | | GGGGTGGGGTATGGGGAGGAGAACCTTCATGGTGGCCCACCTGGCCTGGTTG | |
| | | | TCCAAGCTGTGCCTCGACACATCCTCATCCCCAGCATGGGACACCTCAAGATG | |
| | | | AATAATAATTCACAAAATTTCTGTGAAATCAAATCCAGTTTTAAGAGGAGCCA | |
| | | | CTTATCAAAGAGATTTTAACAGTAGTAAGAAGGCAAAGAATAAACATTTGAT | |
| | | | ATTCAGCAACTGAAAATACCTCACTGTGTTTTCTTGGGGGGAGCAGGGAATTG | |
| | | | ATTACCTTAGCTAGCATGTACAAATTTGACATTGTACTTGAAGATAGTTACATT | |
| | | | GCTTGGAACAGTTGCTTTCTTTCTTTTTTTTTTTTAAATTTTGAAATAGCCACAA | |
| | | | ACTTATAAAACAGTTGGAAGCACAGCAAAAGGACTGTTCTTTTCCTGAGTAAG | |
| | | | TTTCTGATCTAATGTCCTATCACCTTTGAATGGTATTTTTTACAAACAAGAACA | |
| | | | CTGTCCTTCCTAACCATAGTATAGCCTTCAGAAAATTAACATTGATACATTGCT | |
| | | | ACCATCCAATCCTCAGAGCGCCTTCAACATTAGCCCTGTGCATTTAGTTGATAT | |
| | | | GTTTCATGCGCGTCTGTGTGAAGAGACCACCAAACAGGCTTTGTGTGAGCAAT | |
| | | | AAAGCTGTTTATTTCACCTGGGTGCAGGTGAGTTGAGTCCGAAAAGAGAGTCA | |
| | | | GCGAAGGGAGATGGGGTGGGGCCGTTTTATAGAATTTGGGTACGTAAAGGAA | |
| | | | AATTACATTCAAAGGGGGGTTGTTCTCTGGCGGGCAGAGTGGGGGTCACAAG | |
| | | | GTGCTCAGTAGGGGAGCTTTTGAGCCAGGATGAGCCAGAAGAAGGAATTTCA | |
| | | | CAAGACGATGTCATCAGTTAAGGCAGGAACAGGCCATTTTCACTTCTTTTATG | |
| | | | GTGGAATGTCATCAGTTAAGGCAGGAACCGGCCATCTGGATGTGCACGTGCA | |
| | | | GGTCACAGGGGATATGATGGCTTAGCTTGGGCTCAGAGGCCTGACATTCCTGT | |
| | | | CTTCTTATATTAATAAGAAAAATAAAACGAAATAGTGGTAAAGTGTTGGGATG | |
| | | | TCGAAAATTTTTGGGGGTGATATGGAGAGATAATGGGTGATGTTTCTCAGGGC | |
| | | | TGCTTTGAGCGGGATTAGGGGCGGCGTGGGAACCTACAGTGGGAGAGATTAA | |
| | | | GCTGAAGGAAGATTTTGTGGTAAGGGGTGATATTGTGGGGTTGTTAGAAGAA | |
| | | | ACATTTTTCATTTAGAATTATTGGTGATGGCCTGGATACGGTTTTGTATGAATT | |
| | | | GAAAAACTAAACGGAATAAGAGAAGGAGAAAAACAGGTATTAAAGGTCTAA | |
| | | | GAATTGGGAGGACCCGGGACATCTAATTAGAGAGTGCCTAAGGAGGTTCAGC | |
| | | | ATAGCCTTGCCAGTAAAGATTATTTATTTACTTCAAGAGCTAAGAGTGGCAGT | |
| | | | TTGGGGATAGCACCAGGGGATATCAGCTGTGATGACTTGGAGAAACAGTGTA | |
| | | | AACCGGCAGTGTAAACAAGAGCAGGGCATGTATGAGTAGCTGAGAACGGTGA | |
| | | | ATAGGAGTGTGACTAGACAGAAGATGGTAGGGATGACAAGTTTTTCTGGGGC | |
| | | | ACAGTCTAAGTTAGTCTGGTGTCTGGAATGAGACTGGGGCCTAATAAAGAGG | |
| | | | AGCATCCATACAGGAGCTCAAATGGGCTGTACGCTGTAGCATTCTGAGGACA | |
| | | | GGTCTGACTTCTGAGAAGGGAAAGTGGTAAAAGTATTGTCTAGTCCTTTTTAA | |
| | | | GTTGGTGGCTGAGCTTGGTGAGGTGTGTTTTTAAAAGACCATTAGTCTGTTCTA | |
| | | | CTTTTCCTGAAGACTGAGGACCGTAAGGGATATAAAGGTTTCACTGAATACTA | |
| | | | AGAGCTTGAAAAACTGCTTGGCTGATTTGACTAATAAAGGCTGGTCTGTTATC | |
| | | | AGACTGTATAGAGGTGGGAAGGCTAAACTGAGGAATTATGTCTGACAGAAGG | |
| | | | GAAGAAATGACTGCGGTGGCCTTCTCAGACCCTGTAGGAAAGGACTCTACCTA | |
| | | | TATAGTGAAAGTGTCTACCTAGACTAAGAGATATTTTAGTTATCTGACTCGGG | |
| | | | GCATGTTGAGTAAAGCTAATTTGCCAGTCCTGGGTGGGGGCAAATCCTCGAGC | |
| | | | TTGATGTGTAGGGAAGGGAGGGGGCCTGAATAATCCCTGAGGAGTAGTAGAA | |
| | | | TAGCAGATGGAACACTGAGAAGTTATTTCCTTGAGGATAGATTTCCACGATGG | |
| | | | AAAGGAAATG | |
| 57 | IFNG | >NP_000610.2 | SEQ ID NO: 107 | SEQ ID NO: |
| | | interferon gamma | >NM_000619.2 Homo sapiens interferon gamma (IFNG), mRNA | 136 |
| | | precursor [Homo | CACATTGTTCTGATCATCTGAAGATCAGCTATTAGAAGAGAAAGATCAGTTAA | NG_015840.1 |
| | | sapiens] | GTCCTTTGGACCTGATCAGCTTGATACAAGAACTACTGATTTCAACTTCTTTGG | |
| | | MKYTSYILAFQLC | CTTAATTCTCTCGGAAACGATGAAATATACAAGTTATATCTTGGCTTTTCAGCT | |
| | | IVLGSLGCYCQDP | CTGCATCGTTTTGGGTTCTCTTGGCTGTTACTGCCAGGACCCATATGTAAAAGA | |
| | | YVKEAENLKKYF | AGCAGAAAACCTTAAGAAATATTTTAATGCAGGTCATTCAGATGTAGCGGATA | |
| | | NAGHSDVADNGT | ATGGAACTCTTTTCTTAGGCATTTTGAAGAATTGGAAAGAGGAGAGTGACAGA | |
| | | LFLGILKNWKEES | AAAATAATGCAGAGCCAAATTGTCTCCTTTTACTTCAAACTTTTTAAAAACTTT | |
| | | DRKIMQSQIVSFY | AAAGATGACCAGAGCATCCAAAAGAGTGTGGAGACCATCAAGGAAGACATG | |
| | | FKLFKNFKDDQSI | AATGTCAAGTTTTTCAATAGCAACAAAAAGAAACGAGATGACTTCGAAAAGC | |
| | | QKSVETIKEDMNV | TGACTAATTATTCGGTAACTGACTTGAATGTCCAACGCAAAGCAATACATGAA | |
| | | KFFNSNKKKRDDF | CTCATCCAAGTGATGGCTGAACTGTCGCCAGCAGCTAAAACAGGGAAGCGAA | |
| | | EKLTNYSVTDLNV | AAAGGAGTCAGATGCTGTTTCGAGGTCGAAGAGCATCCCAGTAATGGTTGTCC | |
| | | QRKAIHELIQVMA | TGCCTGCAATATTTGAATTTTAAATCTAAATCTATTTATTAATATTTAACATTA | |
| | | ELSPAAKTGKRKR | TTTATATGGGGAATATATTTTTAGACTCATCAATCAAATAAGTATTTATAATAG | |
| | | SWLFRGRRASQ | CAACTTTTGTGTAATGAAAATGAATATCTATTAATATATGTATTATTTATAATT | |
| | | | CCTATATCCTGTGACTGTCTCACTTAATCCTTTGTTTTCTGACTAATTAGGCAA | |
| | | | GGCTATGTGATTACAAGGCTTTATCTCAGGGGCCAACTAGGCAGCCAACCTAA | |
| | | | GCAAGATCCCATGGGTTGTGTGTTTATTTCACTTGATGATACAATGAACACTT | |
| | | | ATAAGTGAAGTGATACTATCCAGTTACTGCCGGTTTGAAAATATGCCTGCAAT | |
| | | | CTGAGCCAGTGCTTTAATGGCATGTCAGACAGAACTTGAATGTGTCAGGTGAC | |
| | | | CCTGATGAAAACATAGCATCTCAGGAGATTTCATGCCTGGTGCTTCCAAATAT | |
| | | | TGTTGACAACTGTGACTGTACCCAAATGGAAAGTAACTCATTTGTTAAAATTA | |
| | | | TCAATATCTAATATATATGAATAAAGTGTAAGTTCACAACAAAAAAAAAAAA | |
| | | | AAAAAAAAAAAAAAAAAA | |
| 58 | GZMB | >NP_004122.2 | SEQ ID NO: 108 | SEQ ID NO: |
| | | granzyme B isoform | >NM_004131.5 Homo sapiens granzyme B (GZMB), transcript variant 1, mRNA | 137 |
| | | 1 preproprotein | GGGGAACATGAAGTCACTGAGCCTGCTCCACCTCTTTCCTCTCCCAAGAGCTA | NG_028340.1 |
| | | [Homo sapiens] | AAAGAGAGCAAGGAGGAAACAACAGCAGCTCCAACCAGGGCAGCCTTCCTGA | |
| | | MQPILLLLAFLLLP | GAAGATGCAACCAATCCTGCTTCTGCTGGCCTTCCTCCTGCTGCCCAGGGCAG | |
| | | RADAGEIIGGHEA | ATGCAGGGGAGATCATCGGGGGACATGAGGCCAAGCCCCACTCCCGCCCCTA | |
| | | KPHSRPYMAYLMI | CATGGCTTATCTTATGATCTGGGATCAGAAGTCTCTGAAGAGGTGCGGTGGCT | |
| | | WDQKSLKRCGGF | TCCTGATACGAGACGACTTCGTGCTGACAGCTGCTCACTGTTGGGGAAGCTCC | |
| | | LIRDDFVLTAAHC | ATAAATGTCACCTTGGGGGCCCACAATATCAAAGAACAGGAGCCGACCCAGC | |
| | | WGSSINVTLGAHN | AGTTTATCCCTGTGAAAAGACCCATCCCCCATCCAGCCTATAATCCTAAGAAC | |
| | | IKEQEPTQQFIPVK | TTCTCCAACGACATCATGCTACTGCAGCTGGAGAGAAAGGCCAAGCGGACCA | |
| | | RPIPHPAYNPKNFS | GAGCTGTGCAGCCCCTCAGGCTACCTAGCAACAAGGCCCAGGTGAAGCCAGG | |
| | | NDIMLLQLERKAK | GCAGACATGCAGTGTGGCCGGCTGGGGGCAGACGGCCCCCCTGGGAAAACAC | |
| | | RTRAVQPLRLPSN | TCACACACACTACAAGAGGTGAAGATGACAGTGCAGGAAGATCGAAAGTGCG | |
| | | KAQVKPGQTCSV | AATCTGACTTACGCCATTATTACGACAGTACCATTGAGTTGTGCGTGGGGGAC | |
| | | AGWGQTAPLGKH | CCAGAGATTAAAAAGACTTCCTTTAAGGGGGACTCTGGAGGCCCTCTTGTGTG | |
| | | SHTLQEVKMTVQ | TAACAAGGTGGCCCAGGGCATTGTCTCCTATGGACGAAACAATGGCATGCCTC | |
| | | EDRKCESDLRHYY | CACGAGCCTGCACCAAAGTCTCAAGCTTTGTACACTGGATAAAGAAAACCAT | |
| | | DSTIELCVGDPEIK | GAAACGCTACTAACTACAGGAAGCAAACTAAGCCCCCGCTGTAATGAAACAC | |
| | | KTSFKGDSGGPLV | CTTCTCTGGAGCCAAGTCCAGATTTACACTGGGAGAGGTGCCAGCAACTGAAT | |
| | | CNKVAQGIVSYGR | AAATACCTCTTAGCTGAGTGGAAAAAAAAAAAAAAAAAA | |
| | | NNGMPPRACTKV | | |
| | | SSFVHWIKKTMKR | | |
| | | Y | | |
| 59 | IDO1 | >NP_002155.1 | SEQ ID NO: 109 | SEQ ID NO: |
| | | indoleamine 2,3- | >NM_002164.5 Homo sapiens indoleamine 2,3-dioxygenase 1 (1DO1), mRNA | 138 |
| | | dioxygenase 1 | AATTTCTCACTGCCCCTGTGATAAACTGTGGTCACTGGCTGTGGCAGCAACTA | NG_028155.1 |
| | | [Homo sapiens] | TTATAAGATGCTCTGAAAACTCTTCAGACACTGAGGGGCACCAGAGGAGCAG | |
| | | MAHAMENSWTIS | ACTACAAGAATGGCACACGCTATGGAAAACTCCTGGACAATCAGTAAAGAGT | |
| | | KEYHIDEEVGFAL | ACCATATTGATGAAGAAGTGGGCTTTGCTCTGCCAAATCCACAGGAAAATCTA | |
| | | PNPQENLPDFYND | CCTGATTTTTATAATGACTGGATGTTCATTGCTAAACATCTGCCTGATCTCATA | |
| | | WMFIAKHLPDHE | GAGTCTGGCCAGCTTCGAGAAAGAGTTGAGAAGTTAAACATGCTCAGCATTG | |
| | | SGQLRERVEKLN | ATCATCTCACAGACCACAAGTCACAGCGCCTTGCACGTCTAGTTCTGGGATGC | |
| | | MLSIDHLTDHKSQ | ATCACCATGGCATATGTGTGGGGCAAAGGTCATGGAGATGTCCGTAAGGTCTT | |
| | | RLARLVLGCITMA | GCCAAGAAATATTGCTGTTCCTTACTGCCAACTCTCCAAGAAACTGGAACTGC | |
| | | YVWGKGHGDVR | CTCCTATITTGGTTTATGCAGACTGTGTCTTGGCAAACTGGAAGAAAAAGGAT | |
| | | KVLPRNIAVPYCQ | CCTAATAAGCCCCTGACTTATGAGAACATGGACGTTTTGTTCTCATTTCGTGAT | |
| | | LSKKLELPPILVYA | GGAGACTGCAGTAAAGGATTCTTCCTGGTCTCTCTATTGGTGGAAATAGCAGC | |
| | | DCVLANWKKKDP | TGCTTCTGCAATCAAAGTAATTCCTACTGTATTCAAGGCAATGCAAATGCAAG | |
| | | NKPLTYENMDVL | AACGGGACACTTTGCTAAAGGCGCTGTTGGAAATAGCTTCTTGCTTGGAGAAA | |
| | | FSFRDGDCSKGFF | GCCCTTCAAGTGTTTCACCAAATCCACGATCATGTGAACCCAAAAGCATTTTT | |
| | | LVSLLVEIAAASAI | CAGTGTTCTTCGCATATATTTGTCTGGCTGGAAAGGCAACCCCCAGCTATCAG | |
| | | KVIPTVFKAMQM | ACGGTCTGGTGTATGAAGGGTTCTGGGAAGACCCAAAGGAGTTTGCAGGGGG | |
| | | QERDTLLKALLEI | CAGTGCAGGCCAAAGCAGCGTCTTTCAGTGCTTTGACGTCCTGCTGGGCATCC | |
| | | ASCLEKALQVFHQ | AGCAGACTGCTGGTGGAGGACATGCTGCTCAGTTCCTCCAGGACATGAGAAG | |
| | | IHDHVNPKAFFSV | ATATATGCCACCAGCTCACAGGAACTTCCTGTGCTCATTAGAGTCAAATCCCT | |
| | | LRIYLSGWKGNPQ | CAGTCCGTGAGTTTGTCCTTTCAAAAGGTGATGCTGGCCTGCGGGAAGCTTAT | |
| | | LSDGLVYEGFWE | GACGCCTGTGTGAAAGCTCTGGTCTCCCTGAGGAGCTACCATCTGCAAATCGT | |
| | | DPKEFAGGSAGQS | GACTAAGTACATCCTGATTCCTGCAAGCCAGCAGCCAAAGGAGAATAAGACC | |
| | | SVFQCFDVLLGIQ | TCTGAAGACCCTTCAAAACTGGAAGCCAAAGGAACTGGAGGCACTGATTTAA | |
| | | QTAGGGHAAQFL | TGAATTTCCTGAAGACTGTAAGAAGTACAACTGAGAAATCCCTTTTGAAGGAA | |
| | | QDMRRYMPPAHR | GGTTAATGTAACCCAACAAGAGCACATTTTATCATAGCAGAGACATCTGTATG | |
| | | NFLCSLESNPSVRE | CATTCCTGTCATTACCCATTGTAACAGAGCCACAAACTAATACTATGCAATGT | |
| | | FVLSKGDAGLREA | TTTACCAATAATGCAATACAAAAGACCTCAAAATACCTGTGCATTTCTTGTAG | |
| | | YDACVKALVSLRS | GAAAACAACAAAAGGTAATTATGTGTAATTATACTAGAAGTTTTGTAATCTGT | |
| | | YHLQIVTKYILIPA | ATCTTATCATTGGAATAAAATGACATTCAATAAATAAAAATGCATAAGATATA | |
| | | SQQPKENKTSEDP | TTCTGTCGGCTGGGCGCGGTGGCTCACGCCTGTAATCCCAGCACTTTGGGAGG | |
| | | SKLEAKGTGGTDL | CCGAGGCGGGCGGATCACAAGGTCAGGAGATCGAGACCATCTTGGCTAACAC | |
| | | MNFLKTVRSTTEK | GGTGAAACCCCGTCTCTACTAAAAATACAAAAAATTAGCCGGGCGCGGTGGC | |
| | | SLLKEG | GGGCACCTGTAGTCCCAGCTACTCGGGAGGCTGAGGCAGGAGAATGGCGTGA | |
| | | | ACCTGGGAGGCGGAGCTTGCAGTGAGCCAAGATTGTGCCACTGCAATCCGGC | |
| | | | CTGGGCTAAAGAGCGGGACTCCGTCTCAAAAAAAAAAAAAAAAAGATATATT | |
| | | | CTGTCATAATAAATAAAAATGCATAAGATATAAAAAAAAAAAAAAA | |
| 60 | NKG7 | >NP_005592.1 | SEQ ID NO: 110 | |
| | | protein NKG7 | >NM_005601.3 Homo sapiens natural killer cell granule protein 7 (NKG7), mRNA | |
| | | [Homo sapiens] | TCATGTGACAAAGCGCAGGACCCCTCACTGCCCCAACTGCTTGCTGTTCTCTCT | |
| | | MELCRSLALLGGS | TTCTTGGGCTCTAAGGACCCAGGAGTCTGGGTGCACAGCCTCCTTCTCTCTGA | |
| | | LGLMFCLIALSTD | GATTCAAGAGTCTGATCAGCAGCCTCTTCCTCCTCCAGGACCCAGAAGCCCTG | |
| | | FWFEAVGPTHSAH | AGCTTATCCCCATGGAGCTCTGCCGGTCCCTGGCCCTGCTGGGGGGCTCCCTG | |
| | | SGLWPTGHGDIIS | GGCCTGATGTTCTGCCTGATTGCTTTGAGCACCGATTTCTGGTTTGAGGCTGTG | |
| | | GYIHVTQTFSIMA | GGTCCCACCCACTCAGCTCACTCGGGCCTCTGGCCAACAGGGCATGGGGACAT | |
| | | VLWALVSVSFLVL | CATATCAGGCTACATCCACGTGACGCAGACCTTCAGCATTATGGCTGTTCTGT | |
| | | SCFPSLFPPGHGPL | GGGCCCTGGTGTCCGTGAGCTTCCTGGTCCTGTCCTGCTTCCCCTCACTGTTCC | |
| | | VSTTAAFAAAISM | CCCCAGGCCACGGCCCGCTTGTCTCAACCACCGCAGCCTTTGCTGCAGCCATC | |
| | | VVAMAVYTSERW | TCCATGGTGGTGGCCATGGCGGTGTACACCAGCGAGCGGTGGGACCAGCCTC | |
| | | DQPPHPQIQTFFS | CACACCCCCAGATCCAGACCTTCTTCTCCTGGTCCTTCTACCTGGGCTGGGTCT | |
| | | WSFYLGWVSAILL | CAGCTATCCTCTTGCTCTGTACAGGTGCCCTGAGCCTGGGTGCTCACTGTGGC | |
| | | LCTGALSLGAHCG | GGTCCCCGTCCTGGCTATGAAACCTTGTGAGCAGAAGGCAAGAGCGGCAAGA | |
| | | GPRPGYETL | TGAGTTTTGAGCGTTGTATTCCAAAGGCCTCATCTGGAGCCTCGGGAAAGTCT | |
| | | | GGTCCCACATCTGCCCGCCCTTCCAGCCCTTCCCCAGCCCCTCCTCTTGTTTCT | |
| | | | TCATTCATTCAACAAAATTTGGCTGGAA | |
| 61 | PRF1 | >NP_001076585.1 | SEQ ID NO: 111 | SEQ ID NO: |
| | | perforin-1 precursor | >NM_001083116.2 Homo sapiens perforM 1 (PRF1), transcript variant 2, mRNA | 139 |
| | | [Homo sapiens] | GGGGCAGGAAGTAGAAGTGATGTGAGTGGTGGCTGGTGCAAGGAGCCACAGT | NG_009615.1 |
| | | MAARLLLLGILLL | GGGCTGCCTGGGGGGCTGATGCCACCATTCCAGGAGCCTCGGTGAAGAGAGG | |
| | | LLPLPVPAPCHTA | ATATCCATCTGTGTAGCCGCTTCTCTATACGGGATTCCAGAGCCCAAGTGCCC | |
| | | ARSECKRSHKFVP | CCTGTCTCTGCAGCTCCATGGCAGCCCGTCTGCTCCTCCTGGGCATCCTTCTCC | |
| | | GAWLAGEGVDVT | TGCTGCTGCCCCTGCCCGTCCCTGCCCCGTGCCACACAGCCGCACGCTCAGAG | |
| | | SLRRSGSFPVDTQ | TGCAAGCGCAGCCACAAGTTCGTGCCTGGTGCATGGCTGGCCGGGGAGGGTG | |
| | | RFLRPDGTCTLCE | TGGACGTGACCAGCCTCCGCCGCTCGGGCTCCTTCCCAGTGGACACACAAAGG | |
| | | NALQEGTLQRLPL | TTCCTGCGGCCCGACGGCACCTGCACCCTCTGTGAAAATGCCCTACAGGAGGG | |
| | | ALTNWRAQGSGC | CACCCTCCAGCGCCTGCCTCTGGCGCTCACCAACTGGCGGGCCCAGGGCTCTG | |
| | | QRHVTRAKVSSTE | GCTGCCAGCGCCATGTAACCAGGGCCAAAGTCAGCTCCACTGAAGCTGTGGC | |
| | | AVARDAARSIRND | CCGGGATGCGGCTCGTAGCATCCGCAACGACTGGAAGGTCGGGCTGGACGTG | |
| | | WKVGLDVTPKPT | ACTCCTAAGCCCACCAGCAATGTGCATGTGTCTGTGGCCGGCTCACACTCACA | |
| | | SNVHVSVAGSHSQ | GGCAGCCAACTTTGCAGCCCAGAAGACCCACCAGGACCAGTACAGCTTCAGC | |
| | | AANFAAQKTHQD | ACTGACACGGTGGAGTGCCGCTTCTACAGTTTCCATGTGGTACACACTCCCCC | |
| | | QYSFSTDTVECRF | GCTGCACCCTGACTTCAAGAGGGCCCTCGGGGACCTGCCCCACCACTTCAACG | |
| | | YSFHVVHTPPLHP | CCTCCACCCAGCCCGCCTACCTCAGGCTTATCTCCAACTACGGCACCCACTTC | |
| | | DFKRALGDLPHHF | ATCCGGGCTGTGGAGCTGGGTGGCCGCATATCGGCCCTCACTGCCCTGCGCAC | |
| | | NASTQPAYLRLIS | CTGCGAGCTGGCCCTGGAAGGGCTCACGGACAACGAGGTGGAGGACTGCCTG | |
| | | NYGTHFIRAVELG | ACTGTCGAGGCCCAGGTCAACATAGGCATCCACGGCAGCATCTCTGCCGAAG | |
| | | GRISALTALRTCEL | CCAAGGCCTGTGAGGAGAAGAAGAAGAAGCACAAGATGACGGCCTCCTTCCA | |
| | | ALEGLTDNEVEDC | CCAAACCTACCGGGAGCGCCACTCGGAAGTGGTTGGCGGCCATCACACCTCC | |
| | | LTVEAQVNIGIHG | ATTAACGACCTGCTGTTCGGGATCCAGGCCGGGCCCGAGCAGTACTCAGCCTG | |
| | | SISAEAKACEEKK | GGTAAACTCGCTGCCCGGCAGCCCTGGCCTGGTGGACTACACCCTGGAACCCC | |
| | | KKHKMTASFHQT | TGCACGTGCTGCTGGACAGCCAGGACCCGCGGCGGGAGGCACTGAGGAGGGC | |
| | | YRERHSEVVGGH | CCTGAGTCAGTACCTGACGGACAGGGCTCGCTGGAGGGACTGCAGCCGGCCG | |
| | | HTSINDLLFGIQAG | TGCCCACCAGGGCGGCAGAAGAGCCCCCGAGACCCATGCCAGTGTGTGTGCC | |
| | | PEQYSAWVNSLPG | ATGGCTCAGCGGTCACCACCCAGGACTGCTGCCCTCGGCAGAGGGGCCTGGC | |
| | | SPGLVDYTLEPLH | CCAGCTGGAGGTGACCTTCATCCAAGCATGGGGCCTGTGGGGGGACTGGTTCA | |
| | | VLLDSQDPRREAL | CTGCCACGGATGCCTATGTGAAGCTCTTCTTTGGTGGCCAGGAGCTGAGGACG | |
| | | RRALSQYLTDRAR | AGCACCGTGTGGGACAATAACAACCCCATCTGGTCAGTGCGGCTGGATTTTGG | |
| | | WIRDCSRPCPPGRQ | GGATGTGCTCCTGGCCACAGGGGGGCCCCTGAGGTTGCAGGTCTGGGATCAG | |
| | | KSPRDPCQCVCHG | GACTCTGGCAGGGACGATGACCTCCTTGGCACCTGTGATCAGGCTCCCAAGTC | |
| | | SAVTTQDCCPRQR | TGGTTCCCATGAGGTGAGATGCAACCTGAATCATGGCCACCTAAAATTCCGCT | |
| | | GLAQLEVTFIQAW | ATCATGCCAGGTGCTTGCCCCACCTGGGAGGAGGCACCTGCCTGGACTATGTC | |
| | | GLWGDWFTATDA | CCCCAAATGCTTCTGGGGGAGCCTCCAGGAAACCGGAGTGGGGCCGTGTGGT | |
| | | YVEILFFGGQELRT | GAGAACAGTGAGCTTGGAAAGGACCAGTATGCTTGGACTGAAGGGGTTCTCA | |
| | | STVWDNNNPIWS | CAGTGGGAGCCAGGGCTGTCTTCGTATTCCCATTAGACCAAGCTTGTCCAACC | |
| | | VRLDFGDVLLATG | CGAGGCCCGCATGCGGCCCAGGATGGCTTTGAATGCGGCCCAACGCAAATTC | |
| | | GPLRLQVWDQDS | GCAAACTTTCTTAAAACATTATGAGTTTCTTTTTGCTATTTTTTTTTTTTTTTTA | |
| | | GRDDDLLGTCDQ | GCTCATCGGCTATCGTTAGTGCTAGTGGATTTTACATGTGGCCCAACACAATT | |
| | | APKSGSHEVRCNL | CTTCTTCCAACGTGGCCCAGAGAAGCCAAAAGATTGGATACGCATCAGACAG | |
| | | NHGHLKFRYHAR | ATGGAAAAGGGAGATTCAGACTGTTTTTCAGGGAGGTGGCTGGGTTTACACGC | |
| | | CLPHLGGGTCLDY | TAATCCCGATTCACCCTGTCCAAACTGCCTAAGCCCTCCGCCATTCTCAAGCCC | |
| | | VPQMLLGEPPGNR | TGCAGTCACAGCTACACAGATCACAGCTTCAGCCAGGAGCTGGGCAGAAGGC | |
| | | SGAVW | CAAGAGGCTGTTCCCACCAGGCTGCTCAGGGCTGGTCTTTTAGGACCCTTCCC | |
| | | | TTGAGCCCTCTATGGTGTGGCAAAGCCTTCATTGCCTTAACTGGAGCCCCATC | |
| | | | AGCTCCAGCTGCTCTGTCTTCTTTGCCCACAATGCTTTGCCCCTGAGACAAATG | |
| | | | GAGGCCTGTCCTGACCTGTCTCACCATGTACATAGCTTGATAAAGGGCCAATA | |
| | | | AATATGATGTTATGGTGAAAAAAAAAAAAAAAAAAAAAA | |
| 62 | CCL5 | >NP_002976.2 C-C | SEQ ID NO: 112 | SEQ ID NO: |
| | | motif chemokine 5 | >NM_002985.2 Homo sapiens C-C motif chemokine ligand 5 (CCL5), transcript variant | 140 |
| | | isoform 1 precursor | 1, mRNA | NG_015990.1 |
| | | [Homo sapiens] | GCTGCAGAGGATTCCTGCAGAGGATCAAGACAGCACGTGGACCTCGCACAGC | |
| | | MKVSAAALAVILI | CTCTCCCACAGGTACCATGAAGGTCTCCGCGGCAGCCCTCGCTGTCATCCTCA | |
| | | ATALCAPASASPY | TTGCTACTGCCCTCTGCGCTCCTGCATCTGCCTCCCCATATTCCTCGGACACCA | |
| | | SSDTTPCCFAYIAR | CACCCTGCTGCTTTGCCTACATTGCCCGCCCACTGCCCCGTGCCCACATCAAG | |
| | | PLPRAHIKEYFYTS | GAGTATTTCTACACCAGTGGCAAGTGCTCCAACCCAGCAGTCGTCTTTGTCAC | |
| | | GKCSNPAVVFVTR | CCGAAAGAACCGCCAAGTGTGTGCCAACCCAGAGAAGAAATGGGTTCGGGAG | |
| | | KNRQVCANPEKK | TACATCAACTCTTTGGAGATGAGCTAGGATGGAGAGTCCTTGAACCTGAACTT | |
| | | WVREYINSLEMS | ACACAAATTTGCCTGTTTCTGCTTGCTCTTGTCCTAGCTTGGGAGGCTTCCCCT | |
| | | | CACTATCCTACCCCACCCGCTCCTTGAAGGGCCCAGATTCTACCACACAGCAG | |
| | | | CAGTTACAAAAACCTTCCCCAGGCTGGACGTGGTGGCTCACGCCTGTAATCCC | |
| | | | AGCACTTTGGGAGGCCAAGGTGGGTGGATCACTTGAGGTCAGGAGTTCGAGA | |
| | | | CCAGCCTGGCCAACATGATGAAACCCCATCTCTACTAAAAATACAAAAAATTA | |
| | | | GCCGGGCGTGGTAGCGGGCGCCTGTAGTCCCAGCTACTCGGGAGGCTGAGGC | |
| | | | AGGAGAATGGCGTGAACCCGGGAGGCGGAGCTTGCAGTGAGCCGAGATCGCG | |
| | | | CCACTGCACTCCAGCCTGGGCGACAGAGCGAGACTCCGTCTCAAAAAAAAAA | |
| | | | AAAAAAAAAAAAATACAAAAATTAGCCGGGCGTGGTGGCCCACGCCTGTAAT | |
| | | | CCCAGCTACTCGGGAGGCTAAGGCAGGAAAATTGTTTGAACCCAGGAGGTGG | |
| | | | AGGCTGCAGTGAGCTGAGATTGTGCCACTTCACTCCAGCCTGGGTGACAAAGT | |
| | | | GAGACTCCGTCACAACAACAACAACAAAAAGCTTCCCCAACTAAAGCCTAGA | |
| | | | AGAGCTTCTGAGGCGCTGCTTTGTCAAAAGGAAGTCTCTAGGTTCTGAGCTCT | |
| | | | GGCTTTGCCTTGGCTTTGCCAGGGCTCTGTGACCAGGAAGGAAGTCAGCATGC | |
| | | | CTCTAGAGGCAAGGAGGGGAGGAACACTGCACTCTTAAGCTTCCGCCGTCTCA | |
| | | | ACCCCTCACAGGAGCTTACTGGCAAACATGAAAAATCGGCTTACCATTAAAGT | |
| | | | TCTCAATGCAACCATAAAAAAAAAA | |
| 63 | IL2RG | >NP_000197.1 | SEQ ID NO: 113 | SEQ ID NO: |
| | | cytokine receptor | >NM_000206.2 Homo sapiens interleukin 2 receptor subunit gamma (1L2RG), mRNA | 140 |
| | | common subunit | AGAGGAAACGTGTGGGTGGGGAGGGGTAGTGGGTGAGGGACCCAGGTTCCTG | NG_021141.1 |
| | | gamma precursor | ACACAGACAGACTACACCCAGGGAATGAAGAGCAAGCGCCATGTTGAAGCCA | |
| | | [Homo sapiens] | TCATTACCATTCACATCCCTCTTATTCCTGCAGCTGCCCCTGCTGGGAGTGGGG | |
| | | MLKPSLPFTSLLFL | CTGAACACGACAATTCTGACGCCCAATGGGAATGAAGACACCACAGCTGATT | |
| | | QLPLLGVGLNTTI | TCTTCCTGACCACTATGCCCACTGACTCCCTCAGTGTTTCCACTCTGCCCCTCC | |
| | | LTPNGNEDTTADF | CAGAGGTTCAGTGTTTTGTGTTCAATGTCGAGTACATGAATTGCACTYGGAAC | |
| | | FLTTMPTDSLSVS | AGCAGCTCTGAGCCCCAGCCTACCAACCTCACTCTGCATTATTGGTACAAGAA | |
| | | TLPLPEVQCFVFN | CTCGGATAATGATAAAGTCCAGAAGTGCAGCCACTATCTATTCTCTGAAGAAA | |
| | | VEYMNCTWNSSS | TCACTTCTGGCTGTCAGTTGCAAAAAAAGGAGATCCACCTCTACCAAACATTT | |
| | | EPQPTNLTLHYWY | GTTGTTCAGCTCCAGGACCCACGGGAACCCAGGAGACAGGCCACACAGATGC | |
| | | KNSDNDKVQKCS | TAAAACTGCAGAATCTGGTGATCCCCTGGGCTCCAGAGAACCTAACACTTCAC | |
| | | HYLFSEEITSGCQL | AAACTGAGTGAATCCCAGCTAGAACTGAACTGGAACAACAGATTCTTGAACC | |
| | | QKKEIHLYQTFVV | ACTGTTTGGAGCACTTGGTGCAGTACCGGACTGACTGGGACCACAGCTGGACT | |
| | | QLQDPREPRRQAT | GAACAATCAGTGGATTATAGACATAAGTTCTCCTTGCCTAGTGTGGATGGGCA | |
| | | QMLKLQNLVIPW | GAAACGCTACACGTTTCGTGTTCGGAGCCGCTTTAACCCACTCTGTGGAAGTG | |
| | | APENLTLHKLSES | CTCAGCATTGGAGTGAATGGAGCCACCCAATCCACTGGGGGAGCAATACTTC | |
| | | QLELNWNNRFLN | AAAAGAGAATCCTTTCCTGTTTGCATTGGAAGCCGTGGTTATCTCTGTTGGCTC | |
| | | HCLEHLVQYRTD | CATGGGATTGATTATCAGCCTTCTCTGTGTGTATTTCTGGCTGGAACGGACGAT | |
| | | WDHSWTEQSVDY | GCCCCGAATTCCCACCCTGAAGAACCTAGAGGATCTTGTTACTGAATACCACG | |
| | | RHKFSLPSVDGQK | GGAACTTTTCGGCCTGGAGTGGTGTGTCTAAGGGACTGGCTGAGAGTCTGCAG | |
| | | RYTFRVRSRFNPL | CCAGACTACAGTGAACGACTCTGCCTCGTCAGTGAGATTCCCCCAAAAGGAG | |
| | | CGSAQHWSEWSH | GGGCCCTTGGGGAGGGGCCTGGGGCCTCCCCATGCAACCAGCATAGCCCCTA | |
| | | PIEIWGSNTSKENP | CTGGGCCCCCCCATGTTACACCCTAAAGCCTGAAACCTGAACCCCAATCCTCT | |
| | | FLFALEAVVISVGS | GACAGAAGAACCCCAGGGTCCTGTAGCCCTAAGTGGTACTAACTTTCCTTCAT | |
| | | MGLIISLLCVYFW | TCAACCCACCTGCGTCTCATACTCACCTCACCCCACTGTGGCTGATTTGGAATT | |
| | | LERTMPRIPTLKN | TTGTGCCCCCATGTAAGCACCCCTTCATTTGGCATTCCCCACTTGAGAATTACC | |
| | | LEDLVTEYHGNFS | CTTTTGCCCCGAACATGTTTTTCTTCTCCCTCAGTCTGGCCCTTCCTTTTCGCAG | |
| | | AWSGVSKGLAES | GATTCTTCCTCCCTCCCTCTTTCCCTCCCTTCCTCTTTCCATCTACCCTCCGATT | |
| | | LQPDYSERLCLVS | GTTCCTGAACCGATGAGAAATAAAGTTTCTGTTGATAATCATCAAAAAAAAAA | |
| | | EIPPKGGALGEGP | AAAAAAAAAAAAAAAAAAAAA | |
| | | GASPCNQHSPYW | | |
| | | APPCYTLKPET | | |
| 64 | CD2 | >NP_001315538.1 | SEQ ID NO: 114 | SEQ ID NO: |
| | | T-cell surface antigen | >NM_001328609.1 Homo sapiens CD2 molecule (CD2), transcript variant 1, mRNA | 141 |
| | | CD2 isoform 1 | AGTCTCACTTCAGTTCCTTTTGCATGAAGAGCTCAGAATCAAAAGAGGAAACC | NG_050908.1 |
| | | precursor [Homo | AACCCCTAAGATGAGCTTTCCATGTAAATTTGTAGCCAGCTTCCTTCTGATTTT | |
| | | sapiens] | CAATGTTTCTTCCAAAGGTGCAGTCTCCAAAGAGATTACGAATGCCTTGGAAA | |
| | | MSFPCKFVASFLLI | CCTGGGGTGCCTTGGGTCAGGACATCAACTTGGACATTCCTAGTTTTCAAATG | |
| | | FNVSSKGAVSKEI | AGTGATGATATTGACGATATAAAATGGGAAAAAACTTCAGACAAGAAAAAGA | |
| | | TNALETWGALGQ | TTGCACAATTCAGAAAAGAGAAAGAGACTTTCAAGGAAAAAGATACATATAA | |
| | | DINLDIPSFQMSDD | GCTATTTAAAAATGGAACTCTGAAAATTAAGCATCTGAAGACCGATGATCAG | |
| | | IDDIKWEKTSDKK | GATATCTACAAGGTATCAATATATGATACAAAAGGAAAAAATGTGTTGGAAA | |
| | | KIAQFRKEKETFK | AAATATTTGATTTGAAGATTCAAGAGAGGGTCTCAAAACCAAAGATCTCCTGG | |
| | | EKDTYKLFKNGTL | ACTTGTATCAACACAACCCTGACCTGTGAGGTAATGAATGGAACTGACCCCGA | |
| | | KIKHLKTDDQDIY | ATTAAACCTGTATCAAGATGGGAAACATCTAAAACTTTCTCAGAGGGTCATCA | |
| | | KVSIYDTKGKNVL | CACACAAGTGGACCACCAGCCTGAGTGCAAAATTCAAGTGCACAGCAGGGAA | |
| | | EKIFDLKIQERVSK | CAAAGTCAGCAAGGAATCCAGTGTCGAGCCTGTCAGCTGTCCAGGAGGCAGC | |
| | | PKISWTCINTTLTC | ATCCTTGGCCAGAGTAATGGGCTCTCTGCCTGGACCCCTCCCAGCCATCCCAC | |
| | | EVMNGTDPELNL | TTCTCTTCCTTTTGCAGAGAAAGGTCTGGACATCTATCTCATCATTGGCATATG | |
| | | YQDGKHLKLSQR | TGGAGGAGGCAGCCTCTTGATGGTCTTTGTGGCACTGCTCGTTTTCTATATCAC | |
| | | VITHKWTTSLSAK | CAAAAGGAAAAAACAGAGGAGTCGGAGAAATGATGAGGAGCTGGAGACAAG | |
| | | FKCTAGNKVSKES | AGCCCACAGAGTAGCTACTGAAGAAAGGGGCCGGAAGCCCCACCAAATTCCA | |
| | | SVEPVSCPGGSILG | GCTTCAACCCCTCAGAATCCAGCAACTTCCCAACATCCTCCTCCACCACCTGG | |
| | | QSNGLSAWTPPSH | TCATCGTTCCCAGGCACCTAGTCATCGTCCCCCGCCTCCTGGACACCGTGTTCA | |
| | | PTSLPFAEKGLDIY | GCACCAGCCTCAGAAGAGGCCTCCTGCTCCGTCGGGCACACAAGTTCACCAGC | |
| | | LIIGICGGGSLLMV | AGAAAGGCCCGCCCCTCCCCAGACCTCGAGTTCAGCCAAAACCTCCCCATGG | |
| | | FVALLVFYITKRK | GGCAGCAGAAAACTCATTGTCCCCTTCCTCTAATTAAAAAAGATAGAAACTGT | |
| | | KQRSRRNDEELET | CTTTTTCAATAAAAAGCACTGTGGATTTCTGCCCTCCTGATGTGCATATCCGTA | |
| | | RAHRVATEERGR | CTTCCATGAGGTGTTTTCTGTGTGCAGAACATTGTCACCTCCTGAGGCTGTGGG | |
| | | KPHQIPASTPQNP | CCACAGCCACCTCTGCATCTTCGAACTCAGCCATGTGGTCAACATCTGGAGTT | |
| | | ATSQHPPPPPGHR | TTTGGTCTCCTCAGAGAGCTCCATCACACCAGTAAGGAGAAGCAATATAAGTG | |
| | | SQAPSHRPPPPGH | TGATTGCAAGAATGGTAGAGGACCGAGCACAGAAATCTTAGAGATTTCTTGTC | |
| | | RVQHQPQKRPPAP | CCCTCTCAGGTCATGTGTAGATGCGATAAATCAAGTGATTGGTGTGCCTGGGT | |
| | | SGTQVHQQKGPPL | CTCACTACAAGCAGCCTATCTGCTTAAGAGACTCTGGAGTTTCTTATGTGCCCT | |
| | | PRPRVQPKPPHGA | GGTGGACACTTGCCCACCATCCTGTGAGTAAAAGTGAAATAAAAGCTTTGACT | |
| | | AENSLSPSSN | AGAAAAAAAAAAAAAAAAAA | |
| 65 | SLAMF6 | >NP_001171643.1 | SEQ ID NO: 115 | |
| | | SLAM family | >NM_001184714.1 Homo sapiens SLAM family member 6 (SLAMF6), transcript variant | |
| | | member 6 isoform 1 | 1, mRNA | |
| | | precursor [Homo | AGTTTATGACAGAAGGGCAAAAACATTGACTGCCTCAAGGTCTCAAGCACCA | |
| | | sapiens] | GTCTTCACCGCGGAAAGCATGTTGTGGCTGTTCCAATCGCTCCTGTTTGTCTTC | |
| | | MLWLFQSLLFVFC | TGCTTTGGCCCAGGGAATGTAGTTTCACAAAGCAGCTTAACCCCATTGATGGT | |
| | | FGPGNVVSQSSLT | GAACGGGATTCTGGGGGAGTCAGTAACTCTTCCCCTGGAGTTTCCTGCAGGAG | |
| | | PLMVNGILGESVT | AGAAGGTCAACTTCATCACTTGGCTTTTCAATGAAACATCTCTTGCCTTCATAG | |
| | | LPLEFPAGEKVNFI | TACCCCATGAAACCAAAAGTCCAGAAATCCACGTGACTAATCCGAAACAGGG | |
| | | TWLFNETSLAFIVP | AAAGCGACTGAACTTCACCCAGTCCTACTCCCTGCAACTCAGCAACCTGAAGA | |
| | | HETKSPEIFIVTNP | TGGAAGACACAGGCTCTTACAGAGCCCAGATATCCACAAAGACCTCTGCAAA | |
| | | KQGKRLNFTQSYS | GCTGTCCAGTTACACTCTGAGGATATTAAGACAACTGAGGAACATACAAGTTA | |
| | | LQLSNLKMEDTGS | CCAATCACAGTCAGCTATTTCAGAATATGACCTGTGAGCTCCATCTGACTTGC | |
| | | YRAQISTKTSAKL | TCTGTGGAGGATGCAGATGACAATGTCTCATTCAGATGGGAGGCCTTGGGAA | |
| | | SSYTLRILRQLRNI | ACACACTTTCAAGTCAGCCAAACCTCACTGTCTCCTGGGACCCCAGGATTTCC | |
| | | QVTNHSQLFQNM | AGTGAACAGGACTACACCTGCATAGCAGAGAATGCTGTCAGTAATTTATCCTT | |
| | | TCELHLTCSVEDA | CTCTGTCTCTGCCCAGAAGCTTTGCGAAGATGTTAAAATTCAATATACAGATA | |
| | | DDNVSFRWEALG | CCAAAATGATTCTGTTTATGGTTTCTGGGATATGCATAGTCTTCGGTTTCATCA | |
| | | NTLSSQPNLTVSW | TACTGCTGTTACTTGTTTTGAGGAAAAGAAGAGATTCCCTATCTTTGTCTACTC | |
| | | DPRISSEQDYTCIA | AGCGAACACAGGGCCCCGCAGAGTCCGCAAGGAACCTAGAGTATGTTTCAGT | |
| | | ENAVSNLSFSVSA | GTCTCCAACGAACAACACTGTGTATGCTTCAGTCACTCATTCAAACAGGGAAA | |
| | | QKLCEDVKIQYTD | CAGAAATCTGGACACCTAGAGAAAATGATACTATCACAATTTACTCCACAATT | |
| | | TKMILFMVSGICIV | AATCATTCCAAAGAGAGTAAACCCACTTTTTCCAGGGCAACTGCCCTTGACAA | |
| | | FGFIILLLLVLRKR | TGTCGTGTAAGTTGCTGAAAGGCCTCAGAGGAATTCGGGAATGACACGTCTTC | |
| | | RDSLSLSTQRTQG | TGATCCCATGAGACAGAACAAAGAACAGGAAGCTTGGTTCCTGTTGTTCCTGG | |
| | | PAESARNLEYVSV | CAACAGAATTTGAATATCTAGGATAGGATGATCACCTCCAGTCCTTCGGACTT | |
| | | SPTNNTVYASVTH | AAACCTGCCTACCTGAGTCAAACACCTAAGGATAACATCATTTCCAGCATGTG | |
| | | SNRETEIWTPREN | GTTCAAATAATATTTTCCAATCCACTTCAGGCCAAAACATGCTAAAGATAACA | |
| | | DTITIYSTINHSKES | CACCAGCACATTGACTCTCTCTTTGATAACTAAGCAAATGGAATTATGGTTGA | |
| | | KPTFSRATALDNV | CAGAGAGTTTATGATCCAGAAGACAACCACTTCTCTCCTTTTAGAAAGCAGCA | |
| | | V | GGATTGACTTATTGAGAAATAATGCAGTGTGTTGGTTACATGTGTAGTCTCTG | |
| | | | GAGTTGGATGGGCCCATCCTGATACAAGTTGAGCATCCCTTGTCTGAAATGCT | |
| | | | TGGGATTAGAAATGTTTCAGATTTCAATTTTTTTTCAGATTTTGGAATATTTGC | |
| | | | ATTATATTTAGCGGTTGAGTATCCAAATCCAAAAATCCAAAATTCAAAATGCT | |
| | | | CCAATAAGCATTTCCCTTGAGTTTCATTGATGTCGATGCAGTGCTCAAAATCTC | |
| | | | AGATTTTGGAGCATTTTGGATATTGGATTTTTGGATTTGGGATGCTCAACTTGT | |
| | | | ACAATGTTTATTAGACACATCTCCTGGGACATACTGCCTAACCTTTTGGAGCCT | |
| | | | TAGTCTCCCAGACTGAAAAAGGAAGAGGATGGTATTACATCAGCTCCATTGTT | |
| | | | TGAGCCAAGAATCTAAGTCATCCCTGACTCCAGTGTCTTTGTCACCAGGCCCT | |
| | | | TTGGACTCTACCTCAGAAATATTTCTTGGACCTTCCACTTCTCCTCCAACTCCT | |
| | | | TGACCACCATCCTGTATCCAACCATCACCACCTCTAACCTGAATCCTACCTTAA | |
| | | | GATCAGAACAGTTGTCCTCACTTTTGTTCTTGTCCCTCTCCAACCCACTCTCCA | |
| | | | CAAGATGGCCAGAGTAATGTTTTTAATATAAATTGGATCCTTCAGTTTCCTGCT | |
| | | | TAAAACCCTGCAGGTTTCCCAATGCACTCAGAAAGAAATCCAGTTTCCATGGC | |
| | | | CCTGGATGGTCTGGCCCACCTCCAGCCTCAGCTAGCATTACCCTTCTGACACTC | |
| | | | TCTATGTAGCCTCCCTGATCTTCTTTCAGCTCCTCTATTAAAGGAAAAGTTCTT | |
| | | | TATGTTAATTATTTACATCTTCCTGCAGGCCCTTCCTCTGCCTGCTGGGGTCCT | |
| | | | CCTATTCTTTAGGTTTAATTTTAAATATGTCACCTCCTAAGAGAAACCTTCCCA | |
| | | | GACCACTCTTTCTAAAATGAATCTTCTAGGCTGGGCATGGTGGCTCACACCTG | |
| | | | TAATCCCTGTACTTTGGGAGGCCAAGGGGGGAGATCACTTGAGGTCAGGAGTT | |
| | | | CAAGACCAGCCTGGCCAACTTGGTGAAACCCCGTCTTTACTAAAAATACAAAA | |
| | | | AAATTAGCCAGGCGTGGTGGTGCACCCCTAAAATCCCAGCTACTTGAGAGACT | |
| | | | GAGGCAGGAGAATCGCTTGAACCCAGGAGGTGGAGGTTCCAGTGAGCCAAAA | |
| | | | TCATGCCAATGTATTCCAGTCTGGGTGACAGAGTGAGACTCTGTCTCAAAAAA | |
| | | | TAAATAAATAAAATAAAATGAAATAGATCTTATAAAAAAAA | |
| 66 | GZMA | >NP_M6135.1 | SEQ ID NO: 116 | SEQ ID NO: |
| | | granzyme A | >NM_006144.3 Homo sapiens granzyme (GZMP), mRNA | 142 |
| | | precursor [Homo | AGATTTTCAGGTTGATTGATGTGGGACAGCAGCCACAATGAGGAACTCCTATA | NG_1127996A |
| | | sapiens] | GATTTCTGGCATCCTCTCTCTCAGTTGTCGTTTCTCTCCTGCTAATTCCTGAAG | |
| | | MRNSYRFLASSLS | ATGTCTGTGAAAAAATTATTGGAGGAAATGAAGTAACTCCTCATTCAAGACCC | |
| | | VVVSLLLIPEDVC | TACATGGTCCTACTTAGTCTTGACAGAAAAACCATCTGTGCTGGGGCTTTGAT | |
| | | EKHGGNEVTPHSR | TGCAAAAGACTGGGTGTTGACTGCAGCTCACTGTAACTTGAACAAAAGGTCCC | |
| | | PYMVLLSLDRKTI | AGGTCATTCTTGGGGCTCACTCAATAACCAGGGAAGAGCCAACAAAACAGAT | |
| | | CAGALIAKDWVL | AATGCTTGTTAAGAAAGAGTTTCCCTATCCATGCTATGACCCAGCCACACGCG | |
| | | TAAHCNLNKRSQ | AAGGTGACCTTAAACTTTTACAGCTGACGGAAAAAGCAAAAATTAACAAATA | |
| | | VILGAHSITREEPT | TGTGACTATCCTTCATCTACCTAAAAAGGGGGATGATGTGAAACCAGGAACCA | |
| | | KQIMLVKKEFPYP | TGTGCCAAGTTGCAGGGTGGGGCAGGACTCACAATAGTGCATCTTGGTCCGAT | |
| | | CYDPATREGDLKL | ACTCTGAGAGAAGTCAATATCACCATCATAGACAGAAAAGTCTGCAATGATC | |
| | | LQLTEKAKINKYV | GAAATCACTATAATTTTAACCCTGTGATTGGAATGAATATGGTTTGTGCTGGA | |
| | | TILHLPKKGDDVK | AGCCTCCGAGGTGGAAGAGACTCGTGCAATGGAGATTCTGGAAGCCCTTTGTT | |
| | | PGTMCQVAGWGR | GTGCGAGGGTGTTTTCCGAGGGGTCACTTCCTTTGGCCTTGAAAATAAATGCG | |
| | | THNSASWSDTLRE | GAGACCCTCGTGGGCCTGGTGTCTATATTCTTCTCTCAAAGAAACACCTCAAC | |
| | | VNITIIDRKVCNDR | TGGATAATTATGACTATCAAGGGAGCAGTTTAAATAACCGTTTCCTTTCATTTA | |
| | | NHYNFNPVIGMN | CTGTGGCTTCTTAATCTTTTCACAAATAAAATCAATTTGCATGACTGTAAAAA | |
| | | MVCAGSLRGGRD | AAAAAAAAAAAAA | |
| | | SCNGDSGSPLLCE | | |
| | | GVFRGVTSFGLEN | | |
| | | KCGDPRGPGVYIL | | |
| | | LSKKHLNWIIMTI | | |
| | | KGAV | | |
| 67 | CD30 | >NP_001234.3 tumor | SEQ ID NO: 117 | SEQ ID NO: |
| | | necrosis factor | >NM_001243.4 Homo sapiens TNF receptor superfamily member 8 (TNFRSF8), | 143 |
| | | receptor superfamily | transcript variant 1, mRNA | NG_029573.2 |
| | | member 8 isoform 1 | ATACGGGAGAACTAAGGCTGAAACCTCGGAGGAACAACCACTTTTGAAGTGA | |
| | | precursor [Homo | CTTCGCGGCGTGCGTTGGGTGCGGACTAGGTGGCCGCGGCGGGAGTGTGCTG | |
| | | sapiens] | GAGCCTGAAGTCCACGCGCGCGGCTGAGAACCGCCGGGACCGCACGTGGGCG | |
| | | MRVLLAALGLLFL | CCGCGCGCTTCCCCCGCTTCCCAGGTGGGCGCCGGCCGCCAGGCCACCTCACG | |
| | | GALRAFPQDRPFE | TCCGGCCCCGGGGATGCGCGTCCTCCTCGCCGCGCTGGGACTGCTGTTCCTGG | |
| | | DTCHGNPSHYYD | GGGCGCTACGAGCCTTCCCACAGGATCGACCCTTCGAGGACACCTGTCATGGA | |
| | | KAVRRCCYRCPM | AACCCCAGCCACTACTATGACAAGGCTGTCAGGAGGTGCTGTTACCGCTGCCC | |
| | | GLFPTQQCPQRPT | CATGGGGCTGTTCCCGACACAGCAGTGCCCACAGAGGCCTACTGACTGCAGG | |
| | | DCRKQCEPDYYL | AAGCAGTGTGAGCCTGACTACTACCTGGATGAGGCCGACCGCTGTACAGCCTG | |
| | | DEADRCTACVTCS | CGTGACTTGTTCTCGAGACGACCTCGTGGAGAAGACGCCGTGTGCATGGAACT | |
| | | RDDLVEKTPCAW | CCTCCCGTGTCTGCGAATGTCGACCCGGCATGTTCTGTTCCACGTCTGCCGTCA | |
| | | NSSRVCECRPGMF | ACTCCTGTGCCCGCTGCTTCTTCCATTCTGTCTGTCCGGCAGGGATGATTGTCA | |
| | | CSTSAVNSCARCF | AGTTCCCAGGCACGGCGCAGAAGAACACGGTCTGTGAGCCGGCTTCCCCAGG | |
| | | FHSVCPAGMIVKF | GGTCAGCCCTGCCTGTGCCAGCCCAGAGAACTGCAAGGAACCCTCCAGTGGC | |
| | | PGTAQKNTVCEPA | ACCATCCCCCAGGCCAAGCCCACCCCGGTGTCCCCAGCAACCTCCAGTGCCAG | |
| | | SPGVSPACASPEN | CACCATGCCTGTAAGAGGGGGCACCCGCCTCGCCCAGGAAGCTGCTTCTAAAC | |
| | | CKEPSSGTIPQAKP | TGACGAGGGCTCCCGACTCTCCCTCCTCTGTGGGAAGGCCTAGTTCAGATCCA | |
| | | TPVSPATSSASTM | GGTCTGTCCCCAACACAGCCATGCCCAGAGGGGTCTGGTGATTGCAGAAAGC | |
| | | PVRGGTRLAQEA | AGTGTGAGCCCGACTACTACCTGGACGAGGCCGGCCGCTGCACGGCCTGCGT | |
| | | ASKLTRAPDSPSS | GAGCTGTTCTCGAGATGACCTTGTGGAGAAGACGCCATGTGCATGGAACTCCT | |
| | | VGRPSSDPGLSPT | CCCGCACCTGCGAATGTCGACCTGGCATGATCTGTGCCACATCAGCCACCAAC | |
| | | QPCPEGSGDCRKQ | TCCTGTGCCCGCTGTGTCCCCTACCCAATCTGTGCAGCAGAGACGGTCACCAA | |
| | | CEPDYYLDEAGRC | GCCCCAGGATATGGCTGAGAAGGACACCACCTTTGAGGCGCCACCCCTGGGG | |
| | | TACVSCSRDDLVE | ACCCAGCCGGACTGCAACCCCACCCCAGAGAATGGCGAGGCGCCTGCCAGCA | |
| | | KTPCAWNSSRTCE | CCAGCCCCACTCAGAGCTTGCTGGTGGACTCCCAGGCCAGTAAGACGCTGCCC | |
| | | CRPGMICATSATN | ATCCCAACCAGCGCTCCCGTCGCTCTCTCCTCCACGGGGAAGCCCGTTCTGGA | |
| | | SCARCVPYPICAA | TGCAGGGCCAGTGCTCTTCTGGGTGATCCTGGTGTTGGTTGTGGTGGTCGGCT | |
| | | ETVTKPQDMAEK | CCAGCGCCTTCCTCCTGTGCCACCGGAGGGCCTGCAGGAAGCGAATTCGGCAG | |
| | | DTTFEAPPLGTQP | AAGCTCCACCTGTGCTACCCGGTCCAGACCTCCCAGCCCAAGCTAGAGCTTGT | |
| | | DCNPTPENGEAPA | GGATTCCAGACCCAGGAGGAGCTCAACGCAGCTGAGGAGTGGTGCGTCGGTG | |
| | | STSPTQSLLVDSQ | ACAGAACCCGTCGCGGAAGAGCGAGGGTTAATGAGCCAGCCACTGATGGAGA | |
| | | ASKTLPIPTSAPVA | CCTGCCACAGCGTGGGGGCAGCCTACCTGGAGAGCCTGCCGCTGCAGGATGC | |
| | | LSSTGKPVLDAGP | CAGCCCGGCCGGGGGCCCCTCGTCCCCCAGGGACCTTCCTGAGCCCCGGGTGT | |
| | | VLFWVILVLVVVV | CCACGGAGCACACCAATAACAAGATTGAGAAAATCTACATCATGAAGGCTGA | |
| | | GSSAFLLCHRRAC | CACCGTGATCGTGGGGACCGTGAAGGCTGAGCTGCCGGAGGGCCGGGGCCTG | |
| | | RKRIRQKLHLCYP | GCGGGGCCAGCAGAGCCCGAGTTGGAGGAGGAGCTGGAGGCGGACCATACCC | |
| | | VQTSQPKLELVDS | CCCACTACCCCGAGCAGGAGACAGAACCGCCTCTGGGCAGCTGCAGCGATGT | |
| | | RPRRSSTQLRSGA | CATGCTCTCAGTGGAAGAGGAAGGGAAAGAAGACCCCTTGCCCACAGCTGCC | |
| | | SVTEPVAEERGLM | TCTGGAAAGTGAGGCCTGGGCTGGGCTGGGGCTAGGAGGGCAGCAGGGTGGC | |
| | | SQPLMETCHSVGA | CTCTGGGAGGCCAGGATGGCACTGTTGGCACCGAGGTTGGGGGCAGAGGCCC | |
| | | AYLESLPLQDASP | ATCTGGCCTGAACTGAGGCTCCAGCATCTAGTGGTGGACCGGCCGGTCACTGC | |
| | | AGGPSSPRDLPEP | AGGGGTCTGGTGGTCTCTGCTTGCATCCCCAACTTAGCTGTCCCCTGACCCAG | |
| | | RVSTEHTNNKIEKI | AGCCTAGGGGATCCGGGGCTTGTACAGAAGAGACAGTCCAAGGGGACTGGAT | |
| | | YIMKADTVIVGTV | CCCAGCAGTGATGTTGGTTGAGGCAGCAAACAGATGGCAGGATGGGCACTGC | |
| | | KAELPEGRGLAGP | CGAGAACAGCATTGGTCCCAGAGCCCTGGGCATCAGACCTTAACCACCAGGC | |
| | | AEPELEEELEADH | CCACAGCCCAGCGAGGGAGAGGTCGTGAGGCCAGCTCCCGGGGCCCCTGTAA | |
| | | TPHYPEQETEPPL | CCCTACTCTCCTCTCTCCCTGGACCTCAGAGGTGACACCCATTGGGCCCTTCCG | |
| | | GSCSDVMLSVEEE | GCATGCCCCCAGTTACTGTAAATGTGGCCCCCAGTGGGCATGGAGCCAGTGCC | |
| | | GKEDPLPTAASGK | TGTGGTTGTTTCTCCAGAGTCAAAAGGGAAGTCGAGGGATGGGGCGTCGTCA | |
| | | | GCTGGCACTGTCTCTGCTGCAGCGGCCACACTGTACTCTGCACTGGTGTGAGG | |
| | | | GCCCCTGCCTGGACTGTGGGACCCTCCTGGTGCTGCCCACCTTCCCTGTCCTGT | |
| | | | AGCCCCCTCGGTGGGCCCAGGGCCTAGGGCCCAGGATCAAGTCACTCATCTCA | |
| | | | GAATGTCCCCACCAATCCCCGCCACAGCAGGCGCCTCGGGTCCCAGATGTCTG | |
| | | | CAGCCCTCAGCAGCTGCAGACCGCCCCTCACCAACCCAGAGAACCTGCTTTAC | |
| | | | TTTGCCCAGGGACTTCCTCCCCATGTGAACATGGGGAACTTCGGGCCCTGCCT | |
| | | | GGAGTCCTTGACCGCTCTCTGTGGGCCCCACCCACTCTGTCCTGGGAAATGAA | |
| | | | GAAGCATCTTCCTTAGGTCTGCCCTGCTTGCAAATCCACTAGCACCGACCCCA | |
| | | | CCACCTGGTTCCGGCTCTGCACGCTTTGGGGTGTGGATGTCGAGAGGCACCAC | |
| | | | GGCCTCACCCAGGCATCTGCTTTACTCTGGACCATAGGAAACAAGACCGTTTG | |
| | | | GAGGTTTCATCAGGATTTTGGGTTTTTCACATTTCACGCTAAGGAGTAGTGGC | |
| | | | CCTGACTTCCGGTCGGCTGGCCAGCTGACTCCCTAGGGCCTTCAGACGTGTAT | |
| | | | GCAAATGAGTGATGGATAAGGATGAGTCTTGGAGTTGCGGGCAGCCTGGAGA | |
| | | | CTCGTGGACTTACCGCCTGGAGGCAGGCCCGGGAAGGCTGCTGTTTACTCATC | |
| | | | GGGCAGCCACGTGCTCTCTGGAGGAAGTGATAGTTTCTGAAACCGCTCAGATG | |
| | | | TTTTGGGGAAAGTTGGAGAAGCCGTGGCCTTGCGAGAGGTGGTTACACCAGA | |
| | | | ACCTGGACATTGGCCAGAAGAAGCTTAAGTGGGCAGACACTGTTTGCCCAGT | |
| | | | GTTTGTGCAAGGATGGAGTGGGTGTCTCTGCATCACCCACAGCCGCAGCTGTA | |
| | | | AGGCACGCTGGAAGGCACACGCCTGCCAGGCAGGGCAGTCTGGCGCCCATGA | |
| | | | TGGGAGGGATTGACATGTTTCAACAAAATAATGCACTTCCTTACCTAGTGGCC | |
| | | | CTTCACACAACTTTTGAATCTCTAAAAATCCATAAAATCCTTAAAGAACTGTA | |
| | | | AAAAAAAAAAAAAAAAAAAAA | |
| 68 | CD3E | >NP_000724.1 T-cell | SEQ ID NO: 118 | SEQ ID NO: |
| | | surface glycoprotein | >NM_000733.3 Homo sapiens CD3e molecule (CD3E), mRNA | 144 |
| | | CD3 epsilon chain | TATTGTCAGAGTCCTCTTGTTTGGCCTTCTAGGAAGGCTGTGGGACCCAGCTTT | NG_007383.1 |
| | | precursor [Homo | CTTCAACCAGTCCAGGTGGAGGCCTCTGCCTTGAACGTTTCCAAGTGAGGTAA | |
| | | sapiens] | AACCCGCAGGCCCAGAGGCCTCTCTACTTCCTGTGTGGGGTTCAGAAACCCTC | |
| | | MQSGTHWRVLGL | CTCCCCTCCCAGCCTCAGGTGCCTGCTTCAGAAAATGAAGTAGTAAGTCTGCT | |
| | | CLLSVGVWGQDG | GGCCTCCGCCATCTTAGTAAAGTAACAGTCCCATGAAACAAAGATGCAGTCG | |
| | | NEEMGGITQTPYK | GGCACTCACTGGAGAGTTCTGGGCCTCTGCCTCTTATCAGTTGGCGTTTGGGG | |
| | | VSISGTTVILTCPQ | GCAAGATGGTAATGAAGAAATGGGTGGTATTACACAGACACCATATAAAGTC | |
| | | YPGSEILWQHNDK | TCCATCTCTGGAACCACAGTAATATTGACATGCCCTCAGTATCCTGGATCTGA | |
| | | NIGGDEDDKNIGS | AATACTATGGCAACACAATGATAAAAACATAGGCGGTGATGAGGATGATAAA | |
| | | DEDHLSLKEFSEL | AACATAGGCAGTGATGAGGATCACCTGTCACTGAAGGAATTTTCAGAATTGG | |
| | | EQSGYYVCYPRGS | AGCAAAGTGGTTATTATGTCTGCTACCCCAGAGGAAGCAAACCAGAAGATGC | |
| | | KPEDANFYLYLRA | GAACTTTTATCTCTACCTGAGGGCAAGAGTGTGTGAGAACTGCATGGAGATGG | |
| | | RVCENCMEMDV | ATGTGATGTCGGTGGCCACAATTGTCATAGTGGACATCTGCATCACTGGGGGC | |
| | | MSVATIVIVDICIT | TTGCTGCTGCTGGTTTACTACTGGAGCAAGAATAGAAAGGCCAAGGCCAAGC | |
| | | GGLLLLVYYWSK | CTGTGACACGAGGAGCGGGTGCTGGCGGCAGGCAAAGGGGACAAAACAAGG | |
| | | NRKAKAKPVTRG | AGAGGCCACCACCTGTTCCCAACCCAGACTATGAGCCCATCCGGAAAGGCCA | |
| | | AGAGGRQRGQNK | GCGGGACCTGTATTCTGGCCTGAATCAGAGACGCATCTGACCCTCTGGAGAAC | |
| | | ERPPPVPNPDYEPI | ACTGCCTCCCGCTGGCCCAGGTCTCCTCTCCAGTCCCCCTGCGACTCCCTGTTT | |
| | | RKGQRDLYSGLN | CCTGGGCTAGTCTTGGACCCCACGAGAGAGAATCGTTCCTCAGCCTCATGGTG | |
| | | QRRI | AACTCGCGCCCTCCAGCCTGATCCCCCGCTCCCTCCTCCCTGCCTTCTCTGCTG | |
| | | | GTACCCAGTCCTAAAATATTGCTGCTTCCTCTTCCTTTGAAGCATCATCAGTAG | |
| | | | TCACACCCTCACAGCTGGCCTGCCCTCTTGCCAGGATATTTATTTGTGCTATTC | |
| | | | ACTCCCTTCCCTTTGGATGTAACTTCTCCGTTCAGTTCCCTCCTTTTCTTGCATG | |
| | | | TAAGTTGTCCCCCATCCCAAAGTATTCCATCTACTTTTCTATCGCCGTCCCCTT | |
| | | | TTGCAGCCCTCTCTGGGGATGGACTGGGTAAATGTTGACAGAGGCCCTGCCCC | |
| | | | GTTCACAGATCCTGGCCCTGAGCCAGCCCTGTGCTCCTCCCTCCCCCAACACT | |
| | | | CCCTACCAACCCCCTAATCCCCTACTCCCTCCACCCCCCCTCCACTGTAGGCCA | |
| | | | CTGGATGGTCATTTGCATCTCCGTAAATGTGCTCTGCTCCTCAGCTGAGAGAG | |
| | | | AAAAAAATAAACTGTATTTGGCTGCAAGAAAAAAAAAAAAAAAAAAAAAA | |
| 69 | CXCR6 | >NP_006555.1 C-X- | SEQ ID NO: 119 | SEQ ID NO: |
| | | C chemokine | >NM_006564.1 Homo sapiens C-X-C motif chemokine receptor 6 (CXCR6), mRNA | 145 |
| | | receptor type 6 | GCAGACCTTGCTTCATGAGCAAGCTCATCTCTGGAACAAACTGGCAAAGCATC | NG_031955.1 |
| | | [Homo sapiens] | TCTGCTGGTGTTCATCAGAACAGACACCATGGCAGAGCATGATTACCATGAAG | |
| | | MAEHDYHEDYGF | ACTATGGGTTCAGCAGTTTCAATGACAGCAGCCAGGAGGAGCATCAAGACTT | |
| | | SSFNDSSQEEHQD | CCTGCAGTTCAGCAAGGTCTTTCTGCCCTGCATGTACCTGGTGGTGTTTGTCTG | |
| | | FLQFSKVFLPCMY | TGGTCTGGTGGGGAACTCTCTGGTGCTGGTCATATCCATCTTCTACCATAAGTT | |
| | | LVVFVCGLVGNSL | GCAGAGCCTGACGGATGTGTTCCTGGTGAACCTACCCCTGGCTGACCTGGTGT | |
| | | VLVISIFYHKLQSL | TTGTCTGCACTCTGCCCTTCTGGGCCTATGCAGGCATCCATGAATGGGTGTTTG | |
| | | TDVFLVNLPLADL | GCCAGGTCATGTGCAAGAGCCTACTGGGCATCTACACTATTAACTTCTACACG | |
| | | VFVCTLPFWAYA | TCCATGCTCATCCTCACCTGCATCACTGTGGATCGTTTCATTGTAGTGGTTAAG | |
| | | GIHEWVFGQVMC | GCCACCAAGGCCTACAACCAGCAAGCCAAGAGGATGACCTGGGGCAAGGTCA | |
| | | KSLLGIYTINFYTS | CCAGCTTGCTCATCTGGGTGATATCCCTGCTGGTTTCCTTGCCCCAAATTATCT | |
| | | MLILTCITVDRFIV | ATGGCAATGTCTTTAATCTCGACAAGCTCATATGTGGTTACCATGACGAGGCA | |
| | | VVKATKAYNQQA | ATTTCCACTGTGGTTCTTGCCACCCAGATGACACTGGGGTTCTTCTTGCCACTG | |
| | | KRMTWGKVTSLLI | CTCACCATGATTGTCTGCTATTCAGTCATAATCAAAACACTGCTTCATGCTGGA | |
| | | WVISLLVSLPQIIY | GGCTTCCAGAAGCACAGATCTCTAAAGATCATCTTCCTGGTGATGGCTGTGTT | |
| | | GNVFNLDKLICGY | CCTGCTGACCCAGATGCCCTTCAACCTCATGAAGTTCATCCGCAGCACACACT | |
| | | HDEAISTVVLATQ | GGGAATACTATGCCATGACCAGCTTTCACTACACCATCATGGTGACAGAGGCC | |
| | | MTLGFFLPLLTMI | ATCGCATACCTGAGGGCCTGCCTTAACCCTGTGCTCTATGCCTTTGTCAGCCTG | |
| | | VCYSVIIKTLLHA | AAGTTTCGAAAGAACTTCTGGAAACTTGTGAAGGACATTGGTTGCCTCCCTTA | |
| | | GGFQKHRSLKIIFL | CCTTGGGGTCTCACATCAATGGAAATCTTCTGAGGACAATTCCAAGACTTTTT | |
| | | VMAVFLLTQMPF | CTGCCTCCCACAATGTGGAGGCCACCAGCATGTTCCAGTTATAGGCCTTGCCA | |
| | | NLMKFIRSTHWEY | GGGTTTCGAGAAGCTGCTCTGGAATTTGCAAGTCATGGCTGTGCCCTCTTGAT | |
| | | YAMTSFHYTIMVT | GTGGTGAGGCAGGCTTTGTTTATAGCTTGCGCATTCTCATGGAGAAGTTATCA | |
| | | EAIAYLRACLNPV | GACACTCTGGCTGGTTTGGAATGCTTCTTCTCAGGCATGAACATGTACTGTTCT | |
| | | LYAFVSLKFRKNF | CTTCTTGAACACTCATGCTGAAAGCCCAAGTAGGGGGTCTAAAATTTTTAAGG | |
| | | WKLVKDIGCLPYL | ACTTTCCTTCCTCCATCTCCAAGAATGCTGAAACCAAGGGGGATGACATGTGA | |
| | | GVSHQWKSSEDN | CTCCTATGATCTCAGGTTCTCCTTGATTGGGACTGGGGCTGAAGGTTGAAGAG | |
| | | SKTFSASHNVEAT | GTGAGCACGGCCAACAAAGCTGTTGATGGTAGGTGGCACACTGGGTGCCCAA | |
| | | SMFQL | GCTCAGAAGGCTCTTCTGACTACTGGGCAAAGAGTGTAGATCAGAGCAGCAG | |
| | | | TGAAAACAAGTGCTGGCACCACCAGGCACCTCACAGAAATGAGATCAGGCTC | |
| | | | TGCCTCACCTTGGGGCTTGACTTTTGTATAGGTAGATGTTCAGATTGCTTTGAT | |
| | | | TAATCCAGAATAACTAGCACCAGGGACTATGAATGGGCAAAACTGAATTATA | |
| | | | AGAGGCTGATAATTCCAGTGGTCCATGGAATGCTTGAAAAATGTGCAAAACA | |
| | | | GCGTTTAAGACTGTAATGAATCTAAGCAGCATTTCTGAAGTGGACTCTTTGGT | |
| | | | GGCTTTGCATTTTAAAAATGAAATTTTCCAATGTCTGCCACACAAACGTATGT | |
| | | | AAATGTATATACCCACACACATACACACATATGTCATATATTACTAGCATATG | |
| | | | AGTTTCATAGCTAAGAAATAAAACTGTTAAAGTCTCCAAACT | |
| 70 | CCR5 | >NP_ 001093638.1 | SEQ ID NO: 120 | SEQ ID NO: |
| | | C-C chemokine | >NM_001100168.1 Homo sapiens C-C motif chemokine receptor 5 (gene/pseudogene) | 146 |
| | | rceptor type 5 | (CCR5), transcript variant B, mRNA | NG_012637.1 |
| | | [Homo sapiens] | CTTCAGATAGATTATATCTGGAGTGAAGAATCCTGCCACCTATGTATCTGGCA | |
| | | MDYQVSSPIYDIN | TAGTCTCATCTGGCCAGAAGAGCTGAGACATCCGTTCCCCTACAAGAAACTCT | |
| | | YYTSEPCQKINVK | CCCCGGGTGGAACAAGATGGATTATCAAGTGTCAAGTCCAATCTATGACATCA | |
| | | QIAARLLPPLYSLV | ATTATTATACATCGGAGCCCTGCCAAAAAATCAATGTGAAGCAAATCGCAGCC | |
| | | FIFGFVGNMLVILI | CGCCTCCTGCCTCCGCTCTACTCACTGGTGTTCATCTTTGGTTTTGTGGGCAAC | |
| | | LINCKRLKSMTDI | ATGCTGGTCATCCTCATCCTGATAAACTGCAAAAGGCTGAAGAGCATGACTGA | |
| | | YLLNLAISDLFFLL | CATCTACCTGCTCAACCTGGCCATCTCTGACCTGTTTTTCCTTCTTACTGTCCCC | |
| | | TVPFWAHYAAAQ | TTCTGGGCTCACTATGCTGCCGCCCAGTGGGACTTTGGAAATACAATGTGTCA | |
| | | WDFGNTMCQLLT | ACTCTTGACAGGGCTCTATTTTATAGGCTTCTTCTCTGGAATCTTCTTCATCAT | |
| | | GLYFIGFFSGIFFII | CCTCCTGACAATCGATAGGTACCTGGCTGTCGTCCATGCTGTGTTTGCTTTAAA | |
| | | LLTIDRYLAVVHA | AGCCAGGACGGTCACCTTTGGGGTGGTGACAAGTGTGATCACTTGGGTGGTGG | |
| | | VFALKARTVTFGV | CTGTGTTTGCGTCTCTCCCAGGAATCATCTTTACCAGATCTCAAAAAGAAGGT | |
| | | VTSVITWVVAVFA | CTTCATTACACCTGCAGCTCTCATTTTCCATACAGTCAGTATCAATTCTGGAAG | |
| | | SLPGIIFTRSQKEG | AATTTCCAGACATTAAAGATAGTCATCTTGGGGCTGGTCCTGCCGCTGCTTGT | |
| | | LHYTCSSHFPYSQ | CATGGTCATCTGCTACTCGGGAATCCTAAAAACTCTGCTTCGGTGTCGAAATG | |
| | | YQFWKNFQTLKIV | AGAAGAAGAGGCACAGGGCTGTGAGGCTTATCTTCACCATCATGATTGTTTAT | |
| | | ILGLVLPLLVMVIC | TTTCTCTTCTGGGCTCCCTACAACATTGTCCTTCTCCTGAACACCTTCCAGGAA | |
| | | YSGILKTLLRCRN | TTCTTTGGCCTGAATAATTGCAGTAGCTCTAACAGGTTGGACCAAGCTATGCA | |
| | | EKKRHRAVRLIFTI | GGTGACAGAGACTCTTGGGATGACGCACTGCTGCATCAACCCCATCATCTATG | |
| | | MIVYFLFWAPYNI | CCTTTGTCGGGGAGAAGTTCAGAAACTACCTCTTAGTCTTCTTCCAAAAGCAC | |
| | | VLLLNTFQEFFGL | ATTGCCAAACGCTTCTGCAAATGCTGTTCTATTTTCCAGCAAGAGGCTCCCGA | |
| | | NNCSSSNRLDQA | GCGAGCAAGCTCAGTTTACACCCGATCCACTGGGGAGCAGGAAATATCTGTG | |
| | | MQVTETLGMTHC | GGCTTGTGACACGGACTCAAGTGGGCTGGTGACCCAGTCAGAGTTGTGCACAT | |
| | | CINPIIYAFVGEKF | GGCTTAGTTTTCATACACAGCCTGGGCTGGGGGTGGGGTGGGAGAGGTCTTTT | |
| | | RNYLLVFFQKHIA | TTAAAAGGAAGTTACTGTTATAGAGGGTCTAAGATTCATCCATTTATTTGGCA | |
| | | KRFCKCCSIFQQE | TCTGTTTAAAGTAGATTAGATCTITTAAGCCCATCAATTATAGAAAGCCAAAT | |
| | | APERASSVYTRST | CAAAATATGTTGATGAAAAATAGCAACCTTTTTATCTCCCCTTCACATGCATC | |
| | | GEQEISVGL | AAGTTATTGACAAACTCTCCCTTCACTCCGAAAGTTCCTTATGTATATTTAAAA | |
| | | | GAAAGCCTCAGAGAATTGCTGATTCTTGAGTTTAGTGATCTGAACAGAAATAC | |
| | | | CAAAATTATTTCAGAAATGTACAACTTTTTACCTAGTACAAGGCAACATATAG | |
| | | | GTTGTAAATGTGTTTAAAACAGGTCTTTGTCTTGCTATGGGGAGAAAAGACAT | |
| | | | GAATATGATTAGTAAAGAAATGACACTTTTCATGTGTGATTTCCCCTCCAAGG | |
| | | | TATGGTTAATAAGTTTCACTGACTTAGAACCAGGCGAGAGACTTGTGGCCTGG | |
| | | | GAGAGCTGGGGAAGCTTCTTAAATGAGAAGGAATTTGAGTTGGATCATCTATT | |
| | | | GCTGGCAAAGACAGAAGCCTCACTGCAAGCACTGCATGGGCAAGCTTGGCTG | |
| | | | TAGAAGGAGACAGAGCTGGTTGGGAAGACATGGGGAGGAAGGACAAGGCTA | |
| | | | GATCATGAAGAACCTTGACGGCATTGCTCCGTCTAAGTCATGAGCTGAGCAGG | |
| | | | GAGATCCTGGTTGGTGTTGCAGAAGGTTTACTCTGTGGCCAAAGGAGGGTCAG | |
| | | | GAAGGATGAGCATTTAGGGCAAGGAGACCACCAACAGCCCTCAGGTCAGGGT | |
| | | | GAGGATGGCCTCTGCTAAGCTCAAGGCGTGAGGATGGGAAGGAGGGAGGTAT | |
| | | | TCGTAAGGATGGGAAGGAGGGAGGTATTCGTGCAGCATATGAGGATGCAGAG | |
| | | | TCAGCAGAACTGGGGTGGATTTGGGTTGGAAGTGAGGGTCAGAGAGGAGTCA | |
| | | | GAGAGAATCCCTAGTCTTCAAGCAGATTGGAGAAACCCTTGAAAAGACATCA | |
| | | | AGCACAGAAGGAGGAGGAGGAGGTTTAGGTCAAGAAGAAGATGGATTGGTG | |
| | | | TAAAAGGATGGGTCTGGTTTGCAGAGCTTGAACACAGTCTCACCCAGACTCCA | |
| | | | GGCTGTCTTTCACTGAATGCTTCTGACTTCATAGATTTCCTTCCCATCCCAGCT | |
| | | | GAAATACTGAGGGGTCTCCAGGAGGAGACTAGATTTATGAATACACGAGGTA | |
| | | | TGAGGTCTAGGAACATACTTCAGCTCACACATGAGATCTAGGTGAGGATTGAT | |
| | | | TACCTAGTAGTCATTTCATGGGITGTTGGGAGGATTCTATGAGGCAACCACAG | |
| | | | GCAGCATTTAGCACATACTACACATTCAATAAGCATCAAACTCTTAGTTACTC | |
| | | | ATTCAGGGATAGCACTGAGCAAAGCATTGAGCAAAGGGGTCCCATAGAGGTG | |
| | | | AGGGAAGCCTGAAAAACTAAGATGCTGCCTGCCCAGTGCACACAAGTGTAGG | |
| | | | TATCATTTTCTGCATTTAACCGTCAATAGGCAAAGGGGGGAAGGGACATATTC | |
| | | | ATTTGGAAATAAGCTGCCTTGAGCCTTAAAACCCACAAAAGTACAATTTACCA | |
| | | | GCCTCCGTATTTCAGACTGAATGGGGGTGGGGGGGGCGCCTTAGGTACTTATT | |
| | | | CCAGATGCCTTCTCCAGACAAACCAGAAGCAACAGAAAAAATCGTCTCTCCCT | |
| | | | CCCTTTGAAATGAATATACCCCTTAGTGTTTGGGTATATTCATTTCAAAGGGA | |
| | | | GAGAGAGAGGTTTTTTTCTGTTCTGTCTCATATGATTGTGCACATACTTGAGAC | |
| | | | TGTTTTGAATTTGGGGGATGGCTAAAACCATCATAGTACAGGTAAGGTGAGGG | |
| | | | AATAGTAAGTGGTGAGAACTACTCAGGGAATGAAGGTGTCAGAATAATAAGA | |
| | | | GGTGCTACTGACTTTCTCAGCCTCTGAATATGAACGGTGAGCATTGTGGCTGT | |
| | | | CAGCAGGAAGCAACGAAGGGAAATGTCTTTCCTTTTGCTCTTAAGTTGTGGAG | |
| | | | AGTGCAACAGTAGCATAGGACCCTACCCTCTGGGCCAAGTCAAAGACATTCTG | |
| | | | ACATCTTAGTATTTGCATATTCTTATGTATGTGAAAGTTACAAATTGCTTGAAA | |
| | | | GAAAATATGCATCTAATAAAAAACACCTTCTAAAATAAAAAAAAAAAAAAAA | |
| | | | AAAAAAAAAAA | |
| 71 | PDCD1 | >NP_005009.2 | SEQ ID NO: 121 | SEQ ID NO: |
| | | programmed cell | >NM_005018.2 Homo sapiens programmed cell death 1 (PDCD1), mRNA | 147 |
| | | death protein 1 | AGTTTCCCTTCCGCTCACCTCCGCCTGAGCAGTGGAGAAGGCGGCACTCTGGT | NG_012110.1 |
| | | precursor [Homo | GGGGCTGCTCCAGGCATGCAGATCCCACAGGCGCCCTGGCCAGTCGTCTGGGC | |
| | | sapiens] | GGTGCTACAACTGGGCTGGCGGCCAGGATGGTTCTTAGACTCCCCAGACAGGC | |
| | | MQIPQAPWPVVW | CCTGGAACCCCCCCACCTTCTCCCCAGCCCTGCTCGTGGTGACCGAAGGGGAC | |
| | | AVLQLGWRPGWF | AACGCCACCTTCACCTGCAGCTTCTCCAACACATCGGAGAGCTTCGTGCTAAA | |
| | | LDSPDRPWNPPTF | CTGGTACCGCATGAGCCCCAGCAACCAGACGGACAAGCTGGCCGCCTTCCCC | |
| | | SPALLVVTEGDNA | GAGGACCGCAGCCAGCCCGGCCAGGACTGCCGCTTCCGTGTCACACAACTGC | |
| | | TFTCSFSNTSESFV | CCAACGGGCGTGACTTCCACATGAGCGTGGTCAGGGCCCGGCGCAATGACAG | |
| | | LNWYRMSPSNQT | CGGCACCTACCTCTGTGGGGCCATCTCCCTGGCCCCCAAGGCGCAGATCAAAG | |
| | | DKLAAFPEDRSQP | AGAGCCTGCGGGCAGAGCTCAGGGTGACAGAGAGAAGGGCAGAAGTGCCCA | |
| | | GQDCRFRVTQLPN | CAGCCCACCCCAGCCCCTCACCCAGGCCAGCCGGCCAGTTCCAAACCCTGGTG | |
| | | GRDFHMSVVRAR | GTTGGTGTCGTGGGCGGCCTGCTGGGCAGCCTGGTGCTGCTAGTCTGGGTCCT | |
| | | RNDSGTYLCGAIS | GGCCGTCATCTGCTCCCGGGCCGCACGAGGGACAATAGGAGCCAGGCGCACC | |
| | | LAPKAQIKESLRA | GGCCAGCCCCTGAAGGAGGACCCCTCAGCCGTGCCTGTGTTCTCTGTGGACTA | |
| | | ELRVTERRAEVPT | TGGGGAGCTGGATTTCCAGTGGCGAGAGAAGACCCCGGAGCCCCCCGTGCCC | |
| | | AHPSPSPRPAGQF | TGTGTCCCTGAGCAGACGGAGTATGCCACCATTGTCTTTCCTAGCGGAATGGG | |
| | | QTLVVGVVGGLL | CACCTCATCCCCCGCCCGCAGGGGCTCAGCTGACGGCCCTCGGAGTGCCCAGC | |
| | | GSLVLLVWVLAVI | CACTGAGGCCTGAGGATGGACACTGCTCTTGGCCCCTCTGACCGGCTTCCTTG | |
| | | CSRAARGTIGARR | GCCACCAGTGTTCTGCAGACCCTCCACCATGAGCCCGGGTCAGCGCATTTCCT | |
| | | TGQPLKEDPSAVP | CAGGAGAAGCAGGCAGGGTGCAGGCCATTGCAGGCCGTCCAGGGGCTGAGCT | |
| | | VFSVDYGELDFQ | GCCTGGGGGCGACCGGGGCTCCAGCCTGCACCTGCACCAGGCACAGCCCCAC | |
| | | WREKTPEPPVPCV | CACAGGACTCATGTCTCAATGCCCACAGTGAGCCCAGGCAGCAGGTGTCACC | |
| | | PEQTEYATIVFPSG | GTCCCCTACAGGGAGGGCCAGATGCAGTCACTGCTTCAGGTCCTGCCAGCACA | |
| | | MGTSSPARRGSAD | GAGCTGCCTGCGTCCAGCTCCCTGAATCTCTGCTGCTGCTGCTGCTGCTGCTGC | |
| | | GPRSAQPLRPEDG | TGCTGCCTGCGGCCCGGGGCTGAAGGCGCCGTGGCCCTGCCTGACGCCCCGGA | |
| | | HCSWPL | GCCTCCTGCCTGAACTTGGGGGCTGGTTGGAGATGGCCTTGGAGCAGCCAAGG | |
| | | | TGCCCCTGGCAGTGGCATCCCGAAACGCCCTGGACGCAGGGCCCAAGACTGG | |
| | | | GCACAGGAGTGGGAGGTACATGGGGCTGGGGACTCCCCAGGAGTTATCTGCT | |
| | | | CCCTGCAGGCCTAGAGAAGTTTCAGGGAAGGTCAGAAGAGCTCCTGGCTGTG | |
| | | | GTGGGCAGGGCAGGAAACCCCTCCACCTTTACACATGCCCAGGCAGCACCTC | |
| | | | AGGCCCTTTGTGGGGCAGGGAAGCTGAGGCAGTAAGCGGGCAGGCAGAGCTG | |
| | | | GAGGCCTTTCAGGCCCAGCCAGCACTCTGGCCTCCTGCCGCCGCATTCCACCC | |
| | | | CAGCCCCTCACACCACTCGGGAGAGGGACATCCTACGGTCCCAAGGTCAGGA | |
| | | | GGGCAGGGCTGGGGTTGACTCAGGCCCCTCCCAGCTGTGGCCACCTGGGTGTT | |
| | | | GGGAGGGCAGAAGTGCAGGCACCTAGGGCCCCCCATGTGCCCACCCTGGGAG | |
| | | | CTCTCCTTGGAACCCATTCCTGAAATTATTTAAAGGGGTTGGCCGGGCTCCCA | |
| | | | CCAGGGCCTGGGTGGGAAGGTACAGGCGTTCCCCCGGGGCCTAGTACCCCCG | |
| | | | CCGTGGCCTATCCACTCCTCACATCCACACACTGCACCCCCACTCCTGGGGCA | |
| | | | GGGCCACCAGCATCCAGGCGGCCAGCAGGCACCTGAGTGGCTGGGACAAGGG | |
| | | | ATCCCCCTTCCCTGTGGTTCTATTATATTATAATTATAATTAAATATGAGAGCA | |
| | | | TGCTAAGGAAAA | |
| 72 | GZMK | >NP_002095.1 | SEQ ID NO: 122 | |
| | | granzyme K | >NM_002104.2 Homo sapiens granzyme K (GZMK), mRNA | |
| | | precursor [Homo | GATCAACACATTTCATCTGGGCTTCTTAAATCTAAATCTTTAAAATGACTAAGT | |
| | | sapiens] | TTTCTTCCTTTTCTCTGTTTTTCCTAATAGTTGGGGCTTATATGACTCATGTGTG | |
| | | MTKFSSFSLFFLIV | TTTCAATATGGAAATTATTGGAGGGAAAGAAGTGTCACCTCATTCCAGGCCAT | |
| | | GAYMTHVCFNME | TTATGGCCTCCATCCAGTATGGCGGACATCACGTTTGTGGAGGTGTTCTGATT | |
| | | IIGGKEVSPHSRPF | GATCCACAGTGGGTGCTGACAGCAGCCCACTGCCAATATCGGTTTACCAAAGG | |
| | | MASIQYGGHHVC | CCAGTCTCCCACTGTGGTTTTAGGCGCACACTCTCTCTCAAAGAATGAGGCCT | |
| | | GGVLIDPQWVLTA | CCAAACAAACACTGGAGATCAAAAAATTTATACCATTCTCAAGAGTTACATCA | |
| | | AHCQYRFTKGQSP | GATCCTCAATCAAATGATATCATGCTGGTTAAGCTTCAAACAGCCGCAAAACT | |
| | | TVVLGAHSLSKNE | CAATAAACATGTCAAGATGCTCCACATAAGATCCAAAACCTCTCTTAGATCTG | |
| | | ASKQTLEIKKFIPF | GAACCAAATGCAAGGTTACTGGCTGGGGAGCCACCGATCCAGATTCATTAAG | |
| | | SRVTSDPQSNDIM | ACCTTCTGACACCCTGCGAGAAGTCACTGTTACTGTCCTAAGTCGAAAACTTT | |
| | | LVKLQTAAKLNK | GCAACAGCCAAAGTTACTACAACGGCGACCCTTTTATCACCAAAGACATGGTC | |
| | | HVKMLHIRSKTSL | TGTGCAGGAGATGCCAAAGGCCAGAAGGATTCCTGTAAGGGTGACTCAGGGG | |
| | | RSGTKCKVTGWG | GCCCCTTGATCTGTAAAGGTGTCTTCCACGCTATAGTCTCTGGAGGTCATGAA | |
| | | ATDPDSLRPSDTL | TGTGGTGTTGCCACAAAGCCTGGAATCTACACCCTGTTAACCAAGAAATACCA | |
| | | REVTVTVLSRKLC | GACTTGGATCAAAAGCAACCTTGTCCCGCCTCATACAAATTAAGTTACAAATA | |
| | | NSQSYYNGDPFIT | ATTTTATTGGATGCACTTGCTTCTTTTTTCCTAATATGCTCGCAGGTTAGAGTT | |
| | | KDMVCAGDAKG | GGGTGTAAGTAAAGCAGAGCACATATGGGGTCCATTTTTGCACTTGTAAGTCA | |
| | | QKDSCKGDSGGPL | TTTTATTAAGGAATCAAGTTCTTTTTCACTTGTATCACTGATGTATTTCTACCAT | |
| | | ICKGVFHAIVSGG | GCTGGTTTTATTCTAAATAAAATTTAGAAGACTCAAAAAAAAAAAAAAAAAA | |
| | | HECGVATKPGIYT | AAAAAAAAAAA | |
| | | LLTKKYQTWIKSN | | |
| | | LVPPHTN | | |
| 73 | CD8A | >NP_001759.3 T-cell | SEQ ID NO: 123 | SEQ ID NO: |
| | | surface glycoprotein | >NM_001768.6 Homo sapiens CD8a molecule (CD8A), transcript variant 1, mRNA | 148 |
| | | CD8 alpha chain | AGCACCCAAGGGCTGGTCAACCAAGCTGGGGGTTGAATTTCCATCCAGCAAT | NG_011608.2 |
| | | isoform 1 precursor | GCAGGCCATGGGAGGCTGCAGCAGTGACGCTGTCAGATCCCCTTTGTGAGAAT | |
| | | [Homo sapiens] | AATAATTTTTATAACAACGTGGCTGGAGGACTGATCAGGAGAGAGACTGGTG | |
| | | MALPVTALLLPLA | TGAATTGAAGGCTGTTGCAATGGCTCCAAGAAGAGATGAGGCTGTGTGGTGA | |
| | | LLLHAARPSQFRV | GTTTAGCCGCTGGATGAAAGGCCGGAAGAATGAGGTCAGCAGCGCACTGACA | |
| | | SPLDRTWNLGETV | CCGACACCCAAAGCTTCGGCTGCTGCCGCTCTCATGGAAATCTCCTGGGGGAA | |
| | | ELKCQVLLSNPTS | GGGAGAGGGTCCTTCCTCGGTGAAAACTGGGGCTGCTCTAGCGAGTTCCTCAG | |
| | | GCSWLFQPRGAA | AAGCGGGCAGGTCGCTAGTTCCTCTTCCTTTTCAGCCCTCAGTGCCCATTTTGC | |
| | | ASPTFLLYLSQNK | CAATAAAAAGTCCCAAGGTGACAGTACAAGAGACGCCTTTAGTGAAGGCAAA | |
| | | PKAAEGLDTQRFS | GGAAGGGACACTCCCCTCCTTTGCTGCCTACTCTCGCCCTCACTTCTTGAAATC | |
| | | GKRLGDTFVLTLS | TTTGGTCTCCCTTCACCCACTCTGTCACTCTCACAAGACAACCATTTCCAAGGA | |
| | | DFRRENEGYYFCS | CTATTTCCAAGCCCTTTTCCTCATCCCCAAACCCGCAGTTTTCAGCTGCCCCCA | |
| | | ALSNSIMYFSHFV | GTTGCCTGGCCAGGCTGCCTCGACGGCCCTATTCACGGGCCCCAGCCTCCTCG | |
| | | PVFLPAKPTTTPAP | CCGGGCTGGAAGGCGACAACCGCGAAAAGGAGGGTGACTCTCCTCGGCGGGG | |
| | | RPPTPAPTIASQPL | GCTTCGGGTGACATCACATCCTCCAAATGCGAAATCAGGCTCCGGGCCGGCCG | |
| | | SLRPEACRPAAGG | AAGGGCGCAACTTTCCCCCCTCGGCGCCCCACCGGCTCCCGCGCGCCTCCCCT | |
| | | AVHTRGLDFACDI | CGCGCCCGAGCTTCGAGCCAAGCAGCGTCCTGGGGAGCGCGTCATGGCCTTAC | |
| | | YIWAPLAGTCGVL | CAGTGACCGCCTTGCTCCTGCCGCTGGCCTTGCTGCTCCACGCCGCCAGGCCG | |
| | | LLSLVITLYCNHR | AGCCAGTTCCGGGTGTCGCCGCTGGATCGGACCTGGAACCTGGGCGAGACAG | |
| | | NRRRVCKCPRPVV | TGGAGCTGAAGTGCCAGGTGCTGCTGTCCAACCCGACGTCGGGCTGCTCGTGG | |
| | | KSGDKPSLSARYV | CTCTTCCAGCCGCGCGGCGCCGCCGCCAGTCCCACCTTCCTCCTATACCTCTCC | |
| | | | CAAAACAAGCCCAAGGCGGCCGAGGGGCTGGACACCCAGCGGTTCTCGGGCA | |
| | | | AGAGGTTGGGGGACACCTTCGTCCTCACCCTGAGCGACTTCCGCCGAGAGAAC | |
| | | | GAGGGCTACTATTTCTGCTCGGCCCTGAGCAACTCCATCATGTACTTCAGCCA | |
| | | | CTTCGTGCCGGTCTTCCTGCCAGCGAAGCCCACCACGACGCCAGCGCCGCGAC | |
| | | | CACCAACACCGGCGCCCACCATCGCGTCGCAGCCCCTGTCCCTGCGCCCAGAG | |
| | | | GCGTGCCGGCCAGCGGCGGGGGGCGCAGTGCACACGAGGGGGCTGGACTTCG | |
| | | | CCTGTGATATCTACATCTGGGCGCCCTTGGCCGGGACTTGTGGGGTCCTTCTCC | |
| | | | TGTCACTGGTTATCACCCTTTACTGCAACCACAGGAACCGAAGACGTGTTTGC | |
| | | | AAATGTCCCCGGCCTGTGGTCAAATCGGGAGACAAGCCCAGCCTTTCGGCGA | |
| | | | GATACGTCTAACCCTGTGCAACAGCCACTACATTACTTCAAACTGAGATCCTT | |
| | | | CCTTTTGAGGGAGCAAGTCCTTCCCTTTCATTTTTTCCAGTCTTCCTCCCTGTGT | |
| | | | ATTCATTCTCATGATTATTATTTTAGTGGGGGCGGGGTGGGAAAGATTACTTTT | |
| | | | TCTTTATGTGTTTGACGGGAAACAAAACTAGGTAAAATCTACAGTACACCACA | |
| | | | AGGGTCACAATACTGTTGTGCGCACATCGCGGTAGGGCGTGGAAAGGGGCAG | |
| | | | GCCAGAGCTACCCGCAGAGTTCTCAGAATCATGCTGAGAGAGCTGGAGGCAC | |
| | | | CCATGCCATCTCAACCTCTTCCCCGCCCGTTTTACAAAGGGGGAGGCTAAAGC | |
| | | | CCAGAGACAGCTTGATCAAAGGCACACAGCAAGTCAGGGTTGGAGCAGTAGC | |
| | | | TGGAGGGACCTTGTCTCCCAGCTCAGGGCTCTTTCCTCCACACCATTCAGGTCT | |
| | | | TTCTTTCCGAGGCCCCTGTCTCAGGGTGAGGTGCTTGAGTCTCCAACGGCAAG | |
| | | | GGAACAAGTACTTCTTGATACCTGGGATACTGTGCCCAGAGCCTCGAGGAGGT | |
| | | | AATGAATTAAAGAAGAGAACTGCCTTTGGCAGAGTTCTATAATGTAAACAAT | |
| | | | ATCAGACTTTTTTTTTTTATAATCAAGCCTAAAATTGTATAGACCTAAAATAAA | |
| | | | ATGAAGTGGTGAGCTTAACCCTGGAAAATGAATCCCTCTATCTCTAAAGAAAA | |
| | | | TCTCTGTGAAACCCCTATGTGGAGGCGGAATTGCTCTCCCAGCCCTTGCATTG | |
| | | | CAGAGGGGCCCATGAAAGAGGACAGGCTACCCCTTTACAAATAGAATTTGAG | |
| | | | CATCAGTGAGGTTAAACTAAGGCCCTCTTGAATCTCTGAATTTGAGATACAAA | |
| | | | CATGTTCCTGGGATCACTGATGACTTTTTATACTTTGTAAAGACAATTGTTGGA | |
| | | | GAGCCCCTCACACAGCCCTGGCCTCTGCTCAACTAGCAGATACAGGGATGAG | |
| | | | GCAGACCTGACTCTCTTAAGGAGGCTGAGAGCCCAAACTGCTGTCCCAAACAT | |
| | | | GCACTTCCTTGCTTAAGGTATGGTACAAGCAATGCCTGCCCATTGGAGAGAAA | |
| | | | AAACTTAAGTAGATAAGGAAATAAGAACCACTCATAATTCTTCACCTTAGGAA | |
| | | | TAATCTCCTGTTAATATGGTGTACATTCTTCCTGATTATTTTCTACACATACAT | |
| | | | GTAAAATATGTCTTTCTTTTTTAAATAGGGTTGTACTATGCTGTTATGAGTGGC | |
| | | | TTTAATGAATAAACATTTGTAGCATCCTCTTTAATGGGTAAACAGCATCCGAA | |
| | | | AAAAAAAAAAAAAAAAAAAAAAAAAAAAAAAAAAAAAAAAAAAAAAAAAA | |
| | | | AAAAAAAAAAAAAAAAAAAAAAAAAAAAAAAAAAAAAAAAAAAAAAAAAA | |
| | | | AAAAAAAAAAAAAAAAAAAAAA | |

What is claimed is:

1. A method comprising:
administering to a subject, a therapeutically effective amount of a B-cell activating factor (BAFF) inhibitor or a salt thereof and a therapeutically effective amount of an immune checkpoint inhibitor or salt thereof.

2. The method of claim 1, wherein said BAFF inhibitor or said salt thereof comprises an antibody, single chain antibody molecule, or an active fragment thereof.

3. The method of claim 2, wherein said antibody or said active fragment thereof is a monoclonal antibody or an active fragment thereof or a polyclonal antibody or an active fragment thereof.

4. The method of claim 3, wherein said antibody or said active fragment thereof is said monoclonal antibody or said active fragment thereof.

5. The method of any one of claims 2-4, wherein said antibody or said active fragment thereof is bispecific.

6. The method of any one of claim 5, wherein said antibody or said active fragment thereof comprises a first domain that binds BAFF or B-cell activating factor receptor (BAFF-R).

7. The method of claim 5 or 6, wherein said antibody or said active fragment thereof comprises a second domain that binds PD-1 or PD-L1.

8. The method of any one of claims 2-7, wherein said antibody or said active fragment thereof is humanized.

9. The method of any one of claims 2-7, wherein said antibody or said active fragment thereof is an IgG, IgE, IgM, IgD, IgA or IgY.

10. The method of any one of claims 2-9, wherein said antibody or said active fragment thereof is a recombinant protein.

11. The method of any one of claims 1-10, wherein said BAFF inhibitor or said salt thereof comprises a heavy chain having at least 80% sequence identity with a sequence shown in SEQ ID NO:150.

12. The method of any one of claims 1-11, wherein said BAFF inhibitor or said salt thereof comprises a heavy chain having at least 90% sequence identity with a sequence shown in SEQ ID NO:150.

13. The method of any one of claims 1-12, wherein said BAFF inhibitor or said salt thereof comprises a light chain having at least 80% sequence identity with a sequence shown in SEQ ID NO: 151.

14. The method of any one of claims 1-13, wherein said BAFF inhibitor or said salt thereof comprises a light chain having at least 90% sequence identity with a sequence shown in SEQ ID NO:151.

15. The method of any one of claims 1-14, wherein said BAFF inhibitor or said salt thereof comprises a light chain variable region having at least 80% sequence identity with a sequence shown in SEQ ID NO:153.

16. The method of any one of claims 1-15, wherein said BAFF inhibitor or said salt thereof comprises a heavy chain variable region having at least 80% sequence identity with a sequence shown in SEQ ID NO:152.

17. The method of any one of claims 1-10, wherein said BAFF inhibitor or said salt thereof comprises a heavy chain having at least 80% sequence identity with a sequence shown in SEQ ID NO:154.

18. The method of any one of claims 1-10 or 17, wherein said BAFF inhibitor or said salt thereof comprises a heavy chain having at least 90% sequence identity with a sequence shown in SEQ ID NO:154.

19. The method of any one of claims 1-10 or 17-18, wherein said BAFF inhibitor or said salt thereof comprises a light chain having at least 80% sequence identity with a sequence shown in SEQ ID NO:155.

20. The method of any one of claims 1-10 or 17-19, wherein said BAFF inhibitor or said salt thereof comprises a light chain having at least 90% sequence identity with a sequence shown in SEQ ID NO: 155.

21. The method of any one of claims 1-10 or 17-20, wherein said BAFF inhibitor or said salt thereof comprises a light chain variable region having at least 80% sequence identity with a sequence shown in SEQ ID NO:157.

22. The method of any one of claims 1-10 or 17-21, wherein said BAFF inhibitor or said salt thereof comprises a heavy chain variable having at least 90% sequence identity with a sequence shown in SEQ ID NO:156.

23. The method of any one of claims 1-10, wherein said BAFF inhibitor said salt thereof comprises as sequence having at least 80% length homology to a sequence shown in SEQ ID NO:1-16 or SEQ ID NO:150-163.

24. The method of any one of claims 1-10, wherein said BAFF inhibitor or said salt thereof is a biosimilar product to a reference product, wherein said reference product comprises tabalumab, atacicept, RCT-18, BAFF/IL-17 bispecific antibody, ardenermin, briobacept, blisibimod, belimumab, MEDI-700, NOV-5, rGel/BlyS, a salt of any one thereof, and any combination thereof.

25. The method of any one of claims 1-10, wherein said BAFF inhibitor or said salt thereof is an interchangeable product to a reference product, wherein said reference product comprises tabalumab, atacicept, RCT-18, BAFF/IL-17 bispecific antibody, ardenermin, briobacept, blisibimod, belimumab, MEDI-700, NOV-5, rGel/BlyS, a salt of any one thereof, and any combination thereof.

26. The method of any one of claims 1-25, wherein said BAFF inhibitor or said salt thereof comprises a post translational modification.

27. The method of claim 26, wherein said post translational modification comprises glycosylation, methylation, phosphorylation, lipidation, acetylation, nitrosylation, or any combination thereof.

28. The method of claim 27, wherein said post translational modification comprises said glycosylation.

29. The method of any one of claims 1-28, wherein said BAFF inhibitor or said salt thereof binds a polypeptide having at least 80% sequence identity with a sequence shown in SEQ ID NOs: 31, 32 or 33.

30. The method of any one of claims 1-28, wherein said BAFF inhibitor or said salt thereof binds a polypeptide having at least 80% sequence identity with a sequence shown in SEQ ID NOs: 149.

31. The method of any one of claims 1-30, wherein said subject have a cancer or is suspected of having said cancer.

32. The method of claim 31, wherein said cancer comprises leukemia, melanoma, prostate cancer, bladder cancer, osteosarcoma, cervical cancer, liver cancer, multiple myeloma, testicular, renal cancer or a combination thereof.

33. The method of any one of claims 31-32, wherein said cancer was at least partially refractive to an immune checkpoint inhibitor or a salt thereof.

34. The method of any one of claims 31-33, wherein said cancer was diagnosed by an assay.

35. The method of claim 34, wherein said assay is a companion diagnostic.

36. The method of any one of claims 1-35, wherein said immune checkpoint inhibitor inhibits PD-1, PD-L1, PD-L2, CTLA-4, A2AR, B7-H3, B7-H4, BTLA, IDO, KIR, LAG3, TIM-3, VISTA, CD160, TIGIT or PSGL-1.

37. The method of claim 36, wherein said immune checkpoint inhibitor or said salt thereof comprises a PD-1 inhibitor or a salt thereof.

38. The method of claim 37, wherein said PD-1 inhibitor or a salt thereof comprises nivolumab, pembrolizumab, pidilizumab, BGB-A31, AMP-224, MEDI680 (AMP-514), PDR001, Cemiplimab or a salt of any one thereof.

39. The method of claim 36, wherein said immune checkpoint inhibitor or a salt thereof comprises a PD-L1 inhibitor or a salt thereof.

40. The method of claim 39, wherein said PD-L1 inhibitor or said salt thereof comprises nivolumab, docetaxel, pembrolizumab, pidilizumab, BGB-A31, MEDI680 (AMP-514), or a salt of any one thereof.

41. The method of any one of claims 1-40, wherein said BAFF inhibitor or said salt thereof is administered intra-arterially, intravenously, intramuscularly, orally, subcutaneously, via inhalation, or any combination thereof.

42. The method of claim 41, wherein said BAFF inhibitor or said salt thereof is administered intravenously.

43. The method of any one of claims 1-42, wherein said therapeutically effective amount of said BAFF inhibitor or said salt thereof comprises a dose of about 0.04 mg/kg to about 20 mg/kg relative to a body weight of said subject.

44. The method of any one of claims 1-43, wherein said immune checkpoint inhibitor or said salt thereof is administered intra-arterially, intravenously, intramuscularly, orally, subcutaneously, via inhalation, or any combination thereof.

45. The method of claim 44, wherein said immune checkpoint inhibitor or said salt thereof is administered intravenously.

46. The method of any one of claims 1-45, wherein said therapeutically effective amount of said immune checkpoint inhibitor or said salt thereof comprises a dose of about 0.04 mg/kg to about 20 mg/kg relative to a body weight of said subject.

47. The method of any one of claims 1-46, wherein said BAFF inhibitor or said salt thereof and said immune checkpoint inhibitor or said salt thereof are administered concurrently.

48. The method of claim 47, wherein said BAFF inhibitor or said salt thereof and said immune checkpoint inhibitor or said salt thereof are administered together in a single composition.

49. The method of any one of claims 1-46, wherein said BAFF inhibitor or said salt thereof and said immune checkpoint inhibitor or said salt thereof are administered sequentially.

50. The method of claim 49, wherein said BAFF inhibitor or said salt thereof is first administered and said immune checkpoint inhibitor or said salt thereof is administered second.

51. The method of claim 49, wherein said immune checkpoint inhibitor or said salt thereof is first administered and said BAFF inhibitor or said salt thereof is administered second.

52. The method of any one of claims 1-51, wherein said administering is performed at least once a month.

53. The method of any one of claims 1-52, wherein said administering is performed at least twice a month.

54. The method of any one of claims 1-53, wherein said subject is a human.

55. The method of any one of claims 1-54, wherein said subject is in need thereof.

56. A pharmaceutical composition for use in the method of any one of claims 1-55, wherein said pharmaceutical composition comprises
a. said BAFF inhibitor or said salt thereof; and
b. said immune checkpoint inhibitor or said salt thereof.

57. The pharmaceutical composition of claim 56, wherein said pharmaceutical composition further comprises a pharmaceutically acceptable excipient.

58. The pharmaceutical composition of any one of claims 56-57, wherein said pharmaceutical composition is in unit dose form.

59. A bispecific antibody comprising:
a. a first domain, wherein said first domain binds BAFF, BAFF-R or a portion thereof, and
b. a second domain, wherein said second domain binds PD-1, PD-L1 or a portion thereof.

60. The bispecific antibody of claim 59, wherein said first domain comprises a heavy chain variable region, light chain variable region, light chain or heavy chain of tabalumab, atacicept, RCT-18, BAFF/IL-17 bispecific antibody, ardenermin, briobacept, blisibimod, belimumab, MEDI-700, NOV-5, rGel/BlyS or a combination thereof.

61. The bispecific antibody of any one of claims 59-60, wherein said second domain comprises a heavy chain variable region, light chain variable region, light chain or heavy chain of nivolumab, docetaxel, pembrolizumab, pidilizumab, BGB-A31, MEDI680, AMP-224, MEDI0680, PDR001, Cemiplimab or a combination thereof.

62. A composition comprising the bispecific antibody of any one of claims 59-61, and a pharmaceutically acceptable carrier.

63. The composition of claim 62 for use in the method of any one of claims 1-55.
